(19) United States
(12) Patent Application Publication
(10) Pub. No.: US 2023/0381321 A1
(43) Pub. Date: Nov. 30, 2023

(54) CAMPTOTHECIN CONJUGATES

(71) Applicant: Seagan Inc.,, Bothell, WA (US)

(72) Inventors: Ryan LYSKI, Bothell, WA (US); Philip MOQUIST, Bothell, WA (US); Nicole DUNCAN, Bothell, WA (US); Scott JEFFREY, Bothell, WA (US)

(21) Appl. No.: 18/185,341

(22) Filed: Mar. 16, 2023

Related U.S. Application Data
(60) Provisional application No. 63/407,609, filed on Sep. 16, 2022.
(60) Provisional application No. 63/321,105, filed on Mar. 17, 2022.

(51) Int. Cl. A61K 47/54; A61K 31/4745; A61K 47/68; A61K 47/60; A61P 35/00

(52) U.S. Cl. CPC A61K 47/545; A61K 31/4745; A61K 47/6851; A61K 47/60; A61P 35/00

(57) ABSTRACT

Antibody conjugates with camptothecin compounds are described, with methods of use and preparations.

### CROSS REFERENCE TO RELATED APPLICATIONS

This application claims priority to and the benefit of U.S. Application No. 63/321,105, filed on Mar. 17, 2022 and U.S. Application No. 63/407,609, filed on Sep. 16, 2022, the disclosures of which are hereby incorporated by reference in their entirety for all purposes.

### REFERENCE TO AN ELECTRONIC SEQUENCE LISTING

The contents of the electronic sequence listing (761682009400SEQLIST.xml; Size: 1,128,239 bytes; and Date of Creation: Mar. 16, 2023) is herein incorporated by reference in its entirety.

### BACKGROUND OF THE INVENTION

Antibodies (mAbs) have been investigated for the targeted delivery of cytotoxic agents to tumor cells. While various drug classes have been evaluated for targeted delivery by antibodies, only a few drug classes have proved sufficiently active as antibody drug conjugates, while having a suitable toxicity profile and other pharmacological properties, to warrant clinical development. One drug class receiving interest is the camptothecins.

The design of Antibody Drug Conjugates (ADCs), by attaching a cytotoxic agent to antibody, typically via a linker, involves consideration of a variety of factors, including the presence of a conjugation handle on the drug for attachment to the linker and linker technology for attaching the drug to an antibody in a conditionally stable manner. The conjugation handle for the parent compound in the class is the C20 hydroxyl functional group in which the linker is attached through a carbonate functional group (e.g., see Walker, M. A. et al. Bioorganic & Medicinal Chemistry Letters (2002) 12(2): 217-219. However, carbonate functional groups typically suffer from hydrolytic instability, which cause premature release of free drug into systemic circulation, which can result in reduced ADC potency, insufficient immunologic specificity of the conjugate and increased toxicity. Therefore, there is a need for camptothecin conjugates engineered for control over drug-linker stability to increase the amount of drug delivered to the desired site of action. The present invention addresses those and other needs.

### BRIEF SUMMARY OF THE INVENTION

The invention provides inter alia, Camptothecin Conjugates, Camptothecin-Linker Compounds and Camptothecin Compounds methods of preparing and using them, and intermediates thereof. The Camptothecin Conjugates of the present invention are stable in circulation, yet capable of inflicting cell death once free drug is released from a Conjugate in the vicinity or within tumor cells.

In one embodiment, a Camptothecin Conjugate is provided having a formula:

L-(Q-D)p

or a salt thereof, wherein
L is a Ligand Unit;
subscript p is an integer of from 1 to 16;
Q is a Linker Unit having a formula selected from the group consisting of:
   Z-A-, -Z-A-RL-, -Z-A-RL-Y-, -Z-A-S*-RL-, -Z-A-S*-RL-Y-, Z-A-S*-W-, -Z-A-S*-W-RL-, -Z-A-S*)-RL-, -Z-A-B(S*)-W-,-Z-A-B(S*)-W-RL- and -Z-A-B(S*)-RL-Y-,
wherein Z is a Stretcher Unit;
A is a bond or a Connector Unit;
B is a Parallel Connector Unit;
S* is a Partitioning Agent;
RL is a Releasable Linker;
W is an Amino Acid Unit;
Y is a Spacer Unit; and
D is a is a Drug Unit having a formula of

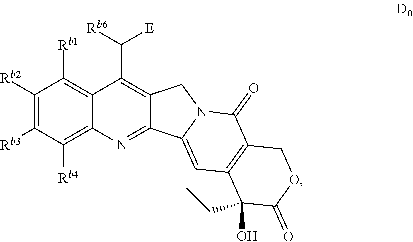

or a salt thereof; wherein;
E is —OR^{b5} or —NR^{b5}R^{b5};
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₆ alkyl-O—C₁-C₆ alkyl-, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, C₁-C₆ hydroxyalkyl-heteroaryl-, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂—C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

Other embodiments as noted above, are Camptothecin-Linker Compounds useful as intermediates for preparing Camptothecin Conjugates, wherein the Camptothecin-Linker Compound is comprised of a Camptothecin and a Linker Unit (Q), wherein the Linker Unit is comprised of a Stretcher Unit precursor (Z′) capable of forming a covalent bond to a targeting ligand that provides for a Ligand Unit, and a Releasable Linker (RL), which in some aspects of Q not having an Amino Acid Unit is a Glycoside (e.g., Glucuronide) Unit.

In another aspect, provided herein are methods of treating cancer comprising administering to a subject in need thereof a Camptothecin Conjugate described herein.

In another aspect, provided herein are kits comprising a Camptothecin Conjugate described herein.

### BRIEF DESCRIPTION OF THE DRAWINGS

FIG. 1A and FIG. 1B show mean tumor volume in a L540cy mouse model over time with administration of one dose of a camptothecin ADC with a Ag4 antibody (FIG. 1A) or an h00 antibody (FIG. 1B) on day 12.

FIG. 2 shows mean tumor volume in EBC-1 mouse models over time with administration of one dose of a glucuronide-based camptothecin ADC with an Ag2 antibody at day 7.

FIG. 3 shows mean tumor volume in OV-90 mouse models over time with administration of one dose of a glucuronide-based camptothecin ADC with an Ag3 antibody at day 17.

FIG. 4 shows mean tumor volume in 786-0 mouse models over time with administration of one dose of a glucuronide-based camptothecin ADC with an Ag5 antibody at day 15.

### DETAILED DESCRIPTION OF THE INVENTION

### Definitions

Unless stated otherwise, the following terms and phrases as used herein are intended to have the following meanings. When trade names are used herein, the trade name includes the product formulation, the generic drug, and the active pharmaceutical ingredient(s) of the trade name product, unless otherwise indicated by context.

The term “antibody” as used herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. The native form of an antibody is a tetramer and consists of two identical pairs of immunoglobulin chains, each pair having one light chain and one heavy chain. In each pair, the light and heavy chain variable regions (V_{L} and V_{H}) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system. (see, e.g., Janeway et al., 2001, Immunol. Biology, 5th Ed., Garland Publishing, New York). An antibody can be of any type (e.g., IgG, IgE, IgM, IgD, and IgA), class (e.g., IgG₁, IgG₂, IgG₃, IgG₄, IgA₁ and IgA₂) or subclass thereof. The antibody can be derived from any suitable species. In some embodiments, the antibody is of human or murine origin. An antibody can be, for example, human, humanized, or chimeric.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

An “intact antibody” is one which comprises an antigen-binding variable region as well as a light chain constant domain (CL) and heavy chain constant domains, C_{H}1, C_{H}2, C_{H}3, and C_{H}4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibits” or “inhibition of” means to reduce by a measurable amount, or to prevent entirely.

The term “therapeutically effective amount” refers to an amount of a conjugate effective to treat a disease or disorder in a mammal. In the case of cancer, the therapeutically effective amount of the conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The term “cytotoxic activity” refers to a cell-killing effect of a drug or Camptothecin Conjugate or an intracellular metabolite of a Camptothecin Conjugate. Cytotoxic activity may be expressed as the IC₅₀ value, which is the concentration (molar or mass) per unit volume at which half the cells survive.

The term “cytostatic activity” refers to an anti-proliferative effect of a drug or Camptothecin Conjugate or an intracellular metabolite of a Camptothecin Conjugate.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant, or animal origin, including synthetic analogs and derivatives thereof.

The term “cytostatic agent” as used herein refers to a substance that inhibits a function of cells, including cell growth or multiplication. Cytostatic agents include inhibitors such as protein inhibitors, e.g., enzyme inhibitors. Cytostatic agents have cytostatic activity.

The terms “cancer” and “cancerous” refer to or describe the physiological condition or disorder in mammals that is typically characterized by unregulated cell growth. A “tumor” comprises one or more cancerous cells.

An “autoimmune disease” as used herein refers to a disease or disorder arising from and directed against an individual's own tissues or proteins.

“Patient” as used herein refers to a subject to whom is administered a Camptothecin Conjugate of the present invention. Patient includes, but are not limited to, a human, rat, mouse, guinea pig, non-human primate, pig, goat, cow, horse, dog, cat, bird, and fowl. Typically, the patient is a rat, mouse, dog, human, or non-human primate, more typically a human.

The terms “treat” or “treatment,” unless otherwise indicated by context, refer to therapeutic treatment and prophylactic wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of: killing tumor cells; inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

“Compound” as the term is used herein, refers to and encompasses the chemical compound itself, either named or represented by structure, and salt form(s) thereof, whether explicitly stated or not, unless context makes clear that such salt forms are to be excluded. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; and when a compound can be associated with two or more solvent molecules, the two or more solvent molecules may be the same or different.

In some instances, a compound of the invention will include an explicit reference to one or more of the above forms, e.g., salts and solvates, which does not imply any solid state form of the compound; however, this reference is for emphasis only, and is not to be construed as excluding any other of the forms as identified above. Furthermore, when explicit reference to a salt and/or solvate form of a compound or a Ligand Drug Conjugate composition is not made, that omission is not to be construed as excluding the salt and/or solvate form(s) of the compound or Conjugate unless context make clear that such salt and/or solvate forms are to be excluded.

The phrase “salt thereof” as the phrase is used herein, refers to a salt form of a compound (e.g., a Drug, a Drug Linker compound or a Ligand Drug Conjugate compound). A salt form of a compound is of one or more internal salt forms and/or involves the inclusion of another molecule such as an acetate ion, a succinate ion or other counterion. The counterion in a salt form of a compound is typically an organic or inorganic moiety that stabilizes the charge on the parent compound. A salt form of a compound has one or more than one charged atom in its structure. In instances where multiple charged atoms are part of the salt form, multiple counter ions and/or multiple charged counter ions are present. Hence, a salt form of a compound typically has one or more charged atoms corresponding to those of the non-salt form of the compound and one or more counterions. In some aspects, the non-salt form of a compound contains at least one amino group or other basic moeity, and accordingly in the presence of an acid, an acid addition salt with the basic moiety is obtained. In other aspects, the non-salt form of a compound contains at least one carboxylic acid group or other acidic moiety, and accordingly in the presence of a base, a carboxylate or other anionic moiety is obtained. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

A pharmaceutically acceptable salt is a salt form of a compound that is suitable for administration to a subject as described herein and in some aspects includes countercations or counteranions as described by P. H. Stahl and C. G. Wermuth, editors, Handbook of Pharmaceutical Salts: Properties, Selection and Use, Weinheim/Zurich:Wiley-VCH/VHCA, 2002.

A Linker Unit is a bifunctional moiety that connects a Camptothecin to a Ligand Unit in a Camptothecin Conjugate. The Linker Units of the present invention have several components (e.g., a Stretcher Unit which in some embodiments will have a Basic Unit; a Connector Unit, that can be present or absent; a Parallel Connector Unit, that can also be present or absent; a Releasable Linker, and a Spacer Unit, that can also be present or absent).

“PEG”, “PEG Unit”, or “polyethylene glycol” as used herein is an organic moiety comprised of repeating ethylene-oxy subunits and may be polydisperse, monodisperse or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein comprises one or multiple polyethylene glycol chains, each comprised of one or more ethyleneoxy subunits, covalently attached to each other. The polyethylene glycol chains can be linked together, for example, in a linear, branched or star shaped configuration. Typically, at least one of the polyethylene glycol chains prior to incorporation into a Camptothecin Conjugate is derivitized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically H or an optionally substituted alkyl such as —CH₃, —CH₂CH₃, or —CH₂CH₂CO₂H. A preferred PEG Unit has a single polyethylene glycol chain with 4 to 24 -CH₂CH₂O— subunits covalently attached in series and terminated at one end with a PEG Capping Unit.

“Halogen” as the term is used herein by itself or in combination with another term, unless otherwise stated or implied by context, refers to fluorine, chlorine, bromine, or iodine and is typically —F or —Cl.

Unless otherwise indicated, the term “alkyl” by itself or as part of another term refers to a substituted or unsubstituted straight chain or branched, saturated or unsaturated hydrocarbon having the indicated number of carbon atoms (e.g., “—C₁-C₈ alkyl” or “—C₁-C₁₀”alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl,-n-propyl, -n-butyl, -n-pentyl, -n-hexyl, -n-heptyl and -n-octyl; while branched —C₃-C₈ alkyls include, but are not limited to, -isopropyl, -sec-butyl, -isobutyl, -tert-butyl, -isopentyl, and -2-methylbutyl; unsaturated —C₂-C₈ alkyls include, but are not limited to, -vinyl, -allyl, -1-butenyl, -2-butenyl, -isobutylenyl, -1 pentenyl, -2 pentenyl, -3-methyl-1-butenyl, -2 methyl-2-butenyl, -2,3 dimethyl-2-butenyl, -1-hexyl, 2-hexyl, -3-hexyl, -acetylenyl, -propynyl, -1 butynyl,-2 butynyl, -1 pentynyl, -2 pentynyl and -3 methyl-1-butynyl. Sometimes an alkyl group is unsubstituted. An alkyl group can be substituted with one or more groups. In other aspects, an alkyl group will be saturated.

Unless otherwise indicated, “alkylene,” by itself of as part of another term, refers to a substituted or unsubstituted saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon).

“Alkenyl” as the term is used herein, by itself or as part of another term, unless otherwise stated or implied by context, refers to an organic moiety, substituent, or group that comprises one or more double bond functional groups (e.g., a —CH═CH— moiety) or 1, 2, 3, 4, 5, or 6 or more, typically 1, 2, or 3 of such functional groups, more typically one such functional group, and in some aspects may be substituted (i.e., is optionally substituted) with an aryl moiety or group such as phenyl, or may contain non-aromatic linked normal, secondary, tertiary or cyclic carbon atoms, i.e., linear, branched, cyclic or any combination thereof as part of the base moiety unless the alkenyl substituent, moiety or group is a vinyl moiety (e.g., a —CH═CH₂ moiety). An alkenyl moiety, group or substituent having multiple double bonds may have the double bonds arranged contiguously (i.e., a 1,3-butadienyl moiety) or non-contiguously with one or more intervening saturated carbon atoms or a combination thereof, provided that a cyclic, contiguous arrangement of double bonds do not form a cyclic conjugated system of 4n+2 electrons (i.e., is not aromatic).

An alkenyl moiety, group or substituent contains at least one sp² carbon atom in which that carbon atom is divalent and is doubly bonded to another organic moiety or Markush structure to which it is associated, or contains at least two sp² carbon atoms in conjugation to each other in which one of the sp² carbon atoms is monovalent and is singly bonded to another organic moiety or Markush structure to which it is associated. Typically, when alkenyl is used as a Markush group (i.e., is a substituent) the alkenyl is singly bonded to a Markush formula or another organic moiety with which it is associated through a sp² carbon of an alkene functional group of the alkenyl moiety. In some aspects, when an alkenyl moiety is specified, species encompasses those corresponding to any of the optionally substituted alkyl or carbocyclyl, groups moieties or substituents described herein that has one or more endo double bonds in which a sp² carbon atom thereof is monovalent and monovalent moieties derived from removal of a hydrogen atom from a sp² carbon of a parent alkene compound. Such monovalent moieties are exemplified without limitation by vinyl (—CH═CH₂), allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, and cyclohexenyl. In some aspects, the term alkenyl encompasses those and/or other linear, cyclic and branched chained, all carbon-containing moieties containing at least one double bond functional group in which one of the sp² carbon atoms is monovalent.

The number of carbon atoms in an alkenyl moiety is defined by the number of sp² carbon atoms of the alkene functional group(s) that defines it as an alkenyl substituent and the total number of contiguous non-aromatic carbon atoms appended to each of these sp² carbons not including any carbon atom of the other moiety or Markush structure for which the alkenyl moiety is a variable group and carbon atoms from any optional substituent to the alkenyl moiety. That number ranges from 1 to 50 or 1 to 30, typically 1 to 20 or 1 to 12, more typically, 1 to 8, 1 to 6, or 1 to 4 carbon atoms when the double bond functional group is doubly bonded to a Markush structure (e.g. ═CH₂), or ranges from 2 to 50, typically 2 to 30, 2 to 20, or 2 to 12, more typically 2 to 8, 2 to 6, or 2 to 4 carbon atoms, when the double bond functional group is singly bonded to the Markush structure (e.g., —CH═CH₂). For example, C₂-C₈ alkenyl or C₂-C₈ alkenyl means an alkenyl moiety containing 2, 3, 4, 5, 6, 7, or 8 carbon atoms in which at least two are sp² carbon atoms in conjugation with each other with one of these carbon atoms being monovalent, and C₂-C₆ alkenyl or C₂-C₆ alkenyl means an alkenyl moiety containing 2, 3, 4, 5, or 6 carbon atoms in which at least two are sp² carbons that are in conjugation with each other with one of these carbon atoms being monovalent. In some aspects, an alkenyl substituent or group is a C₂-C₆ or C₂-C₄ alkenyl moiety having only two sp² carbons that are in conjugation with each other with one of these carbon atoms being monovalent, and in other aspects that alkenyl moiety is unsubstituted or is substituted with 1 to 4 or more, typically 1 to 3, more typically 1 or 2, independently selected moieties as disclosed herein, including substituents as defined herein for optional substituents, excluding alkyl, arylalkyl, heteroarylalkyl, alkenyl, alkynyl, and any other moiety when the substituted alkenyl would differ by the number of contiguous non-aromatic carbon atoms relative to the unsubstituted alkenyl, wherein the substitution(s) may be at any of the alkenyl moiety's contiguous sp² carbon and sp³ carbon atoms, if any. Typically, an alkenyl substituent is a C₂-C₆ or C₂-C₄ alkenyl moiety having only two sp² carbons that are in conjugation with each other. When the number of carbon atoms is not indicated, an alkenyl moiety has from 2 to 8 carbon atoms.

“Alkenylene” as the term is used herein, by itself of as part of another term, unless otherwise stated or implied by context, refers to an organic moiety, substituent or group that comprises one or more double bond moieties, as previously described for alkenyl, of the stated number of carbon atoms and has two radical centers derived by the removal of two hydrogen atoms from the same or two different sp² carbon atoms of an alkene functional group or removal of two hydrogen atoms from two separate alkene functional groups in a parent alkene. In some aspects, an alkenylene moiety is that of an alkenyl radical as described herein in which a hydrogen atom has been removed from the same or different sp² carbon atom of a double bond functional group of the alkenyl radical, or from a sp² carbon from a different double bonded moiety to provide a diradical. Typically, alkenylene moieties encompass diradicals containing the structure of —C═C— or —C═C—X¹—C═C— wherein X¹ is absent or is an optionally substituted saturated alkylene as defined herein, which is typically a C₁-C₆ alkylene, which is more typically unsubstituted. The number of carbon atoms in an alkenylene moiety is defined by the number of sp² carbon atoms of its alkene functional group(s) that defines it as an alkenylene moiety and the total number of contiguous non-aromatic carbon atoms appended to each of its sp² carbons not including any carbon atoms of the other moiety or Markush structure in which the alkenyl moiety is a present as a variable group. That number, unless otherwise specified, ranges from 2 to 50 or 2 to 30, typically from 2 to 20 or 2 to 12, more typically from 2 to 8, 2 to 6, or 2 to 4 carbon atoms. For example, C₂-C₈ alkenylene or C₂-C₈ alkenylene means an alkenylene moiety containing 2, 3, 4, 5, 6, 7, or 8 carbon atoms, in which at least two are sp² carbons in which one is divalent or both are monovalent, that are in conjugation with each other and C₂-C₆ alkenylene or C₂-C₆ alkenylene means an alkenyl moiety containing 2, 3, 4, 5, or 6 carbon atoms in which at least two are sp² carbons, in which at least two are sp² carbons in which one is divalent or both are monovalent, that are in conjugation with each other. In some aspects, an alkenylene moiety is a C₂-C₆ or C₂-C₄ alkenylene having two sp² carbons that are in conjugation with each other in which both sp² carbon atoms are monovalent, and in some aspects is unsubstituted. When the number of carbon atoms is not indicated, an alkenylene moiety has from 2 to 8 carbon atoms and is unsubstituted or substituted in the same manner described for an alkenyl moiety.

“Alkynyl” as the term is used herein, by itself or as part of another term, unless otherwise stated or implied by context, refers to an organic moiety, substituent or group that comprises one or more triple bond functional groups (e.g., a —C═C— moiety) or 1, 2, 3, 4, 5, or 6 or more, typically 1, 2, or 3 of such functional groups, more typically one such functional group, and in some aspects may be substituted (i.e., is optionally substituted) with an aryl moiety such as phenyl, or by an alkenyl moiety or linked normal, secondary, tertiary or cyclic carbon atoms, i.e., linear, branched, cyclic or any combination thereof unless the alkynyl substituent, moiety or group is —C═CH). An alkynyl moiety, group or substituent having multiple triple bonds may have the triple bonds arranged contiguously or non-contiguously with one or more intervening saturated or unsaturated carbon atoms or a combination thereof, provided that a cyclic, contiguous arrangement of triple bonds do not form a cyclic conjugated system of 4n+2 electrons (i.e., is not aromatic).

An alkynyl moiety, group or substituent contains at least two sp carbon atom in which the carbon atoms are conjugation to each other and in which one of the sp carbon atoms is singly bonded, to another organic moiety or Markush structure to which it is associated. When alkynyl is used as a Markush group (i.e., is a substituent) the alkynyl is singly bonded to a Markush formula or another organic moiety with which it is associated through a triple-bonded carbon (i.e., a sp carbon) of a terminal alkyne functional group. In some aspects when an alkynyl moiety, group or substituent is specified, species encompasses are any of the optionally substituted alkyl or carbocyclyl, groups moieties or substituents described herein that has one or more endo triple bonds and monovalent moieties derived from removal of a hydrogen atom from a sp carbon of a parent alkyne compound. Such monovalent moieties are exemplified without limitation by —C≡CH, and —C≡C—CH₃, and C EC-Ph.

The number of carbon atoms in an alkynyl substituent is defined by the number of sp carbon atoms of the alkene functional group that defines it as an alkynyl substituent and the total number of contiguous non-aromatic carbon atoms appended to each of these sp carbons not including any carbon atom of the other moiety or Markush structure for which the alkenyl moiety is a variable group. That number can vary ranging from 2 to 50, typically 2 to 30, 2 to 20, or 2 to 12, more typically 2 to 8, 2 to 6, or 2 to 4 carbon atoms, when the triple bond functional group is singly bonded to the Markush structure (e.g., —CH≡CH). For example, C₂-C₈ alkynyl or C₂-C₈ alkynyl means an alkynyl moiety containing 2, 3, 4, 5, 6, 7, or 8 carbon atoms in which at least two are sp carbon atoms in conjugation with each other with one of these carbon atoms being monovalent, and C₂-C₆ alkynyl or C₂-C₆ alkynyl means an alkynyl moiety containing 2, 3, 4, 5, or 6 carbon atoms in which at least two are sp carbons that are in conjugation with each other with one of these carbon atoms being monovalent. In some aspects, an alkynyl substituent or group is a C₂-C₆ or C₂-C₄ alkynyl moiety having two sp carbons that are in conjugation with each other with one of these carbon atoms being monovalent, and in other aspects that alkynyl moiety is unsubstituted. When the number of carbon atoms is not indicated, an alkynyl moiety, group or substituent has from 2 to 8 carbon atoms. An alkynyl moiety may be substituted or unsubstituted in the same manner as described for an alkenyl moiety, except that substitution at the monovalent sp carbon is not permitted.

The term “Prodrug” as used herein refers to a less biologically active or inactive compound which is transformed within the body into a more biologically active compound via a chemical or biological process (i.e., a chemical reaction or an enzymatic biotransformation). Typically, a biologically active compound is rendered less biologically active (i.e., is converted to a prodrug) by chemically modifying the compound with a prodrug moiety. In some aspects, the prodrug is a Type II prodrug, which are bioactivated outside cells, e.g., in digestive fluids, or in the body's circulation system, e.g., in blood. Exemplary prodrugs are esters and β-D-glucopyranosides.

Unless otherwise indicated, “aryl,” by itself or as part of another term, means a substituted or unsubstituted monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene,” by itself or as part of another term, is an aryl group as defined above which has two covalent bonds (i.e., it is divalent) and can be in the ortho, meta, or para orientations as shown in the following structures, with phenyl as the exemplary group:

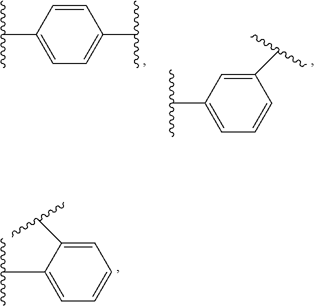 [SMILES: *c1ccc(*)cc1 ; *c1cccc(*)c1 ; *c1ccccc1*]

Unless otherwise indicated, a “C₃-C₈ heterocycle,” by itself or as part of another term, refers to a monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Heterocycles in which all the ring atoms are involved in aromaticity are referred to as heteroaryls and otherwise are referred to heterocarbocycles.

Unless otherwise noted, the heterocycle is attached to its pendant group at any heteroatom or carbon atom that results in a stable structure. As such a heteroaryl may be bonded through an aromatic carbon of its aromatic ring system, referred to as a C-linked heteroaryl, or through a non-double-bonded N atom (i.e., not ═N—) in its aromatic ring system, which is referred to as an N-linked heteroaryl. Thus, nitrogen-containing heterocycles may be C-linked or N-linked and include pyrrole moieties, such as pyrrol-1-yl (N-linked) and pyrrol-3-yl (C-linked), and imidazole moieties such as imidazol-1-yl and imidazol-3-yl (both N-linked), and imidazol-2-yl, imidazol-4-yl and imidazol-5-yl moieties (all of which are C-linked).

Unless otherwise indicated, a “C₃-C₈ heteroaryl,” is an aromatic C₃-C₈ heterocycle in which the subscript denotes the total number of carbons of the cyclic ring system of the heterocycle or the total number of aromatic carbons of the aromatic ring system of the heteroaryl and does not implicate the size of the ring system or the presence or absence of ring fusion. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

When explicitly given, the size of the ring system of a heterocycle or heteroaryl is indicated by the total number of atoms in the ring. For example, designation as a 5- or 6-membered heteroaryl indicates the total number or aromatic atoms (i.e., 5 or 6) in the heteroaromatic ring system of the heteroaryl but does not imply the number of aromatic heteroatoms or aromatic carbons in that ring system. Fused heteroaryls are explicitly stated or implied by context as such and are typically indicated by the number of aromatic atoms in each aromatic ring that are fused together to make up the fused heteroaromatic ring system. For example, a 5,6-membered heteroaryl is an aromatic 5-membered ring fused to an aromatic 6-membered ring in which one or both rings have aromatic heteroatom(s) or where a heteroatom is shared between the two rings.

A heterocycle fused to an aryl or heteroaryl such that the heterocycle remains non-aromatic and is part of a larger structure through attachment with the non-aromatic portion of the fused ring system is an example of an optionally substituted heterocycle in which the heterocycle is substituted by ring fusion with the aryl or heteroaryl. Likewise, an aryl or heteroaryl fused to heterocycle or carbocycle that is part of a larger structure through attachment with the aromatic portion of the fused ring system is an example of an optionally substituted aryl or heterocycle in which the aryl or heterocycle is substituted by ring fusion with the heterocycle or carbocycle.

Unless otherwise indicated, “C₃-C₈ heterocyclo,” by itself or as part of another term, refers to a C₃-C₈ heterocyclic defined above wherein one of the hydrogen atoms of the heterocycle is replaced with a bond (i.e., it is divalent). Unless otherwise indicated, a “C₃-C₈ heteroarylene,” by itself or as part of another term, refers to a C₃-C₈ heteroaryl group defined above wherein one of the heteroaryl group's hydrogen atoms is replaced with a bond (i.e., it is divalent).

Unless otherwise indicated, a “C₃-C₈ carbocycle,” by itself or as part of another term, is a 3-, 4-, 5-, 6-, 7-, or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo,” by itself or as part of another term, refers to a C₃-C₈ carbocycle group defined above wherein another one of the carbocycle groups' hydrogen atoms is replaced with a bond (i.e., it is divalent).

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si, and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si can be placed at any position of the heteroalkyl group, including the position at which the alkyl group is attached to the remainder of the molecule.

Examples of heteroalkyls include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

Unless otherwise indicated, “aminoalkyl” by itself or in combination with another term means a heteroalkyl wherein an alkyl moiety as defined herein is substituted with an amino, alkylamino, dialkylamino or cycloalkylamino group. Exemplary non-limiting aminoalkyls are —CH₂NH₂, —CH₂CH₂NH₂, —CH₂CH₂NHCH₃, and —CH₂CH₂N(CH₃)₂ and further includes branched species such as —CH(CH₃)NH₂ and —C(CH₃)CH₂NH₂ in the (R)- or (S)-configuration. Alternatively, an aminoalkyl is an alkyl moiety, group, or substituent as defined herein wherein a sp³ carbon other than the radical carbon has been replaced with an amino or alkylamino moiety wherein its sp³ nitrogen replaces the sp³ carbon of the alkyl provided that at least one sp^{a} carbon remains. When referring to an aminoalkyl moiety as a substituent to a larger structure or another moiety the aminoalkyl is covalently attached to the structure or moiety through the carbon radical of the alkyl moiety of the aminoalkyl.

“Hydroxyalkyl” as the term is used herein by itself or in combination with another term, unless otherwise stated or implied by context, referes to an alkyl moiety, group, or substituent having a hydroxyl radical in place of one or more hydrogen atoms. In some aspects, one or two hydrogen atoms are replaced with a hydroxyl substituent in a hydroxyalkyl group. A hydroxyalkyl is typically denoted by the number of contiguous carbon atoms of its alkyl or alkylene moiety. Thus, a C₁ hydroxyalkyl is exemplified without limitation by —CH₂OH, and a C₂ hydroxyalkyl is exemplified without limitation by —CH₂CH₂OH or —CH₂(OH)CH₃.

“Haloalkyl” as the term is used herein by itself or in combination with another term, unless otherwise stated or implied by context, referes to an alkyl moiety, group, or substituent having a halogen in place of one or more hydrogen atoms. In some aspects, one or two hydrogen atoms are replaced with a halogen in a haloalkyl group. A haloalkyl is typically denoted by the number of contiguous carbon atoms of its alkyl or alkylene moiety. Thus, a C₁ haloalkyl is exemplified without limitation by —CH₂F, —CH₂Cl, —CH₂Br, or —CH₂I, and a C₂ haloalkyl is exemplified without limitation by —CH₂CH₂F, —CH₂CH₂Cl, —CH₂CH₂Br, —CH₂CH₂I, —CH(F)CH₃, —CH(Cl)CH₃, —CH(Br)CH₃, or —CH(I)CH₃. In some embodiments, the term “haloalkyl” refers to an alkyl moiety, group, or substituent having halogens in place of two or more hydrogen atoms. For example, a C₁ haloalkyl is also exemplified without limitation by —CHF₂, —CHCl₂, —CHBr₂, or —CHI₂, and a C₂ haloalkyl is exemplified without limitation by —CH₂CHF₂, —CH₂CHCl₂, —CH₂CHBr₂, —CH₂CHI₂, —CF₂CH₃, —CCl₂CH₃, —CBr₂CH₃, or —Cl₂CH₃. In some embodiments, the term “haloalkyl” refers to an alkyl moiety, group, or substituent having halogens in place of all hydrogen atoms. In some embodiments, the term “haloalkyl” encompasses fully halogenated alkyl moieties, groups, or substituents. For example, a C₁ haloalkyl is also exemplified without limitation by —CF₃, —CCl₃, —CBr₃, or —CI₃.

Unless otherwise indicated “alkylamino” and “cycloalkylamino” by itself or in combination with another term means an alkyl or cycloalkyl radical, as described herein, wherein the radical carbon of the alkyl or cycloalkyl radical has been replaced with a nitrogen radical, provided that at least one sp³ carbon remains. In those instances where the alkylamino is substituted at its nitrogen with another alkyl moiety the resulting substituted radical is sometimes referred to as a dialkylamino moiety, group, or substituent wherein the alkyl moieties substituting nitrogen are independently selected.

Exemplary and non-limiting amino, alkylamino, and dialkylamino substituents, include those having the structure of —N(R′)₂, wherein R′ in these examples are independently selected from hydrogen or C₁-6 alkyl, typically hydrogen or methyl, whereas in cycloalkyl amines, which are included in heterocycloalkyls, both R′ together with the nitrogen to which they are attached define a heterocyclic ring. When both R′ are hydrogen or alkyl, the moiety is sometimes described as a primary amino group and a tertiary amine group, respectively. When one R′ is hydrogen and the other is alkyl, then the moiety is sometimes described as a secondary amino group. Primary and secondary alkylamino moieties are more reactive as nucleophiles towards carbonyl-containing electrophilic centers whereas tertiary amines are more basic.

“Substituted alkyl” and “substituted aryl” mean alkyl and aryl, respectively, in which one or more hydrogen atoms, typically one, are each independently replaced with a substituent. Typical substituents include, but are not limited to a —X, —R′, —OH, —OR′, —SR^{a}, —N(R′)₂, —N(R′)₃, ═NR′, —CX₃, —CN, —NO₂, —NR′C(═O)R′, —C(═O)R′, —C(═O)N(R)₂, —S(═O)₂R′, —S(═O)₂NR′, —S(═O)R′, —OP(═O)(OR′)₂, —P(═O)(OR′)₂, —PO₃═, PO₃H₂, —C(═O)R′, —C(═S)R′, —CO₂R′, —CO₂′, —C(═S)OR′, —C(═O)SR′, —C(═S)SR′, —C(═O)N(R′)₂, —C(═S)N(R′)₂, and —C(═NR)N(R′)₂, where each X is independently selected from the group consisting of a halogen: —F, —Cl, —Br, and —I; and wherein each R; is independently selected from the group consisting of —H, —C₁-C₂₀ alkyl, —C₆-C_{2,3} aryl, —C₃-C₁₄ heterocycle, a protecting group, and a prodrug moiety.

More typically substituents are selected from the group consisting of —X, —R′, —OH, —OR′, —SR^{a}, —N(R′)₂, —N(R′)₃, ═NR′, —NR′C(═O)R′, —C(═O)R′, —C(═O)N(R′)₂, —S(═O)₂R′, —S(═O)₂NR′, —S(═O)R′, —C(═O)R′, —C(═S)R′, —C(═O)N(R′)₂, —C(═S)N(R′)₂, and —C(═NR)N(R′)₂, wherein each X is independently selected from the group consisting of —F and —Cl, or are selected from the group consisting of —X, —R′, —OH, —OR′, —N(R′)₂, —N(R′)₃, —NR′C(═O)R′, —C(═O)N(R′)₂, —S(═O)₂R′, —S(═O)₂NR′, —S(═O)R′, —C(═O)R′, —C(═O)N(R′)₂, —C(═NR)N(R′)₂, a protecting group, and a prodrug moiety, wherein each X is —F; and wherein each R; is independently selected from the group consisting of hydrogen, —C₁-C₂₀ alkyl, —C₆-C_{2,3} aryl, —C₃-C₁₄ heterocycle, a protecting group, and a prodrug moiety.

In some aspects, an alkyl substituent is selected from the group consisting —N(R′)₂, —N(R′)₃ and —C(═NR)N(R′)₂, wherein R; is selected from the group consisting of hydrogen and —C₁-C₂₀ alkyl. In other aspects, alkyl is substituted with a series of ethyleneoxy moieties to define a PEG Unit. Alkylene, carbocycle, carbocyclo, arylene, heteroalkyl, heteroalkylene, heterocycle, heterocyclo, heteroaryl, and heteroarylene groups as described above may also be similarly substituted.

“Protecting group” as used herein, means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, Wiley Interscience.

Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by —OR^{PR}, wherein R^{PR} is a protecting group for hydroxyl, wherein hydroxyl is typically protected as an ester (e.g. acetate, propionate, or benzoate). Other protecting groups for hydroxyl avoid interfering with the nucleophilicity of organometallic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS), and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. By way of example and not limitation, a suitable protecting group may include those previously described for protecting functional groups. A suitable protecting group is sometimes a protecting group used in peptide coupling reactions.

“Electron withdrawing group” as used herein means a functional group or electronegative atom that draws electron density away from an atom to which it is bonded either inductively and/or through resonance, whichever is more dominant (i.e., a functional group or atom may be electron withdrawing inductively but may overall be electron donating through resonance) and tends to stabilize anions or electron-rich moieties. The electron withdrawing effect is typically transmitted inductively, albeit in attenuated form, to other atoms attached to the bonded atom that has been made electron deficient by the electron withdrawing group (EWG), thus affecting the electrophilicity of a more remote reactive center. Exemplary electron withdrawing groups include, but are not limited to —C(═O), —CN, —NO₂, —CX₃, —X, —C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃⁺, wherein X is —F, —Br, —Cl, or —I, and R′ in some aspects is, at each occurrence, independently selected from the group consisting of hydrogen and C₁₋₆ alkyl, and certain O-linked moieties as described herein such as acyloxy.

Exemplary EWGs can also include aryl groups (e.g., phenyl) depending on substitution and certain heteroaryl groups (e.g., pyridine). Thus, the term “electron withdrawing groups” also includes aryls or heteroaryls that are further substituted with electron withdrawing groups. Typically, electron withdrawing groups on aryls or heteroaryls are —C(═O), —CN, —NO₂, —CX₃, and —X, wherein X independently selected is halogen, typically —F or —Cl. Depending on their substituents, an alkyl moiety may also be an electron withdrawing group.

“Succinimide moiety” as used herein, refers to an organic moiety comprised of a succinimide ring system, which is present in one type of Stretcher Unit (Z) that is typically further comprised of an alkylene-containing moiety bonded to the imide nitrogen of that ring system. A succinimide moiety typically results from Michael addition of a sulfhydryl group of a Ligand Unit to the maleimide ring system of a Stretcher Unit precursor (Z′). A succinimide moiety is therefore comprised of a thio-substituted succinimide ring system and when present in a Camptothecin Conjugate has its imide nitrogen substituted with the remainder of the Linker Unit of the Camptothecin Conjugate and is optionally substituted with substituent(s) that were present on the maleimide ring system of Z′.

“Acid-amide moiety,” as used herein refers to succinic acid having an amide substituent that results from the thio-substituted succinimide ring system of a succinimide moiety having undergone breakage of one of its carbonyl-nitrogen bonds by hydrolysis. Hydrolysis resulting in a succinic acid-amide moiety provides a Linker Unit less likely to suffer premature loss of the Ligand Unit to which it is bonded through elimination of the antibody-thio substituent. Hydrolysis of the succinimide ring system of the thio-substituted succinimide moiety is expected to provide regiochemical isomers of acid-amide moieties that are due to differences in reactivity of the two carbonyl carbons of the succinimide ring system attributable at least in part to any substituent present in the maleimide ring system of the Stretcher Unit precursor and to the thio substituent introduced by the targeting ligand.

In many instances, the assembly of the conjugates, linkers and components described herein will refer to reactive groups. A “reactive group” or RG is a group that contains a reactive site (RS) capable of forming a bond with either the components of the Linker unit (i.e., A, W, Y) or the Camptothecin D. RS is the reactive site within a Reactive Group (RG). Non-limiting examples of reactive groups include sulfhydryl groups to form disulfide bonds or thioether bonds; aldehyde, ketone, or hydrazine groups to form hydrazone bonds; carboxylic or amino groups to form peptide bonds; carboxylic or hydroxy groups to form ester bonds; sulfonic acids to form sulfonamide bonds; alcohols to form carbamate bonds; and amines to form sulfonamide bonds or carbamate bonds.

The following table is illustrative of Reactive Groups, Reactive Sites, and exemplary functional groups that can form after reaction of the reactive site. The table is not limiting. One of skill in the art will appreciate that the noted R′ and R^{x}′ portions in the table are effectively any organic moiety (e.g., an alkyl group, aryl group, heteroaryl group, or substituted alkyl, aryl, or heteroaryl, group) which is compatible with the bond formation provided in converting RG to one of the Exemplary Functional Groups. It will also be appreciated that, as applied to the embodiments of the present invention, R′ may represent one or more components of the self-stabilizing linker or optional secondary linker, as the case may be, and R^{x}′ may represent one or more components of the optional secondary linker, Camptothecin, stabilizing unit, or detection unit, as the case may be.

| | | Exemplary |
|---|---|---|
| RG | RS | Functional Groups |
| 1) R′—SH | —S— | R′—S—R″, R′—S—S—R″ |
| 2) R′—C(═O)OH | —C(═O)— | R′—C(═O)NH—R″ |
| 3) R′—C(═O)ONHS | —C(═O)— | R′—C(═O)NH—R″ |
| 4) R′S(═O)₂—OH | —S(═O)₂— | R′S(═O)₂NH—R″ |
| 5) R′—CH₂—X | —CH₂— | R′—CH₂—S—R″ |
| (X is Br, I, Cl) | | |
| 6) R′—NH₂ | —N— | R′—NHC(═O)R″ |

### EMBODIMENTS

A number of embodiments of the invention are described below, which are not meant to limit the invention in any way, are followed by a more detailed discussion of the components that make up the conjugates. One of skill in the art will understand that each of the conjugates identified and any of the selected embodiments thereof is meant to include the full scope of each component and linker.

### Camptothecin Conjugates

In one embodiment, provided herein are camptothecin conjugates having a formula:

L-(Q-D)p

or a salt thereof, wherein
L is a Ligand Unit;
the subscript p is an integer of from 1 to 16;
Q is a Linker Unit having a formula selected from the group consisting of:
-Z-A-, -Z-A-RL-; -Z-A-RL-Y-; Z-A-S*-W-; -Z-A-S*-RL-; -Z-A-B(S*)-RL-;
-Z-A-S*-W-RL-, -Z-A-S*-RL-Y-; and -Z-A-B(S*)-RL-Y-;
wherein Z is a Stretcher Unit,
A is a bond or a Connector Unit;
B is a Parallel Connector Unit;
is a Partitioning Agent;
W is a Peptide Unit;
RL is a Releasable Unit;
Y is a Spacer Unit; and
D is a Drug Unit having a formula of

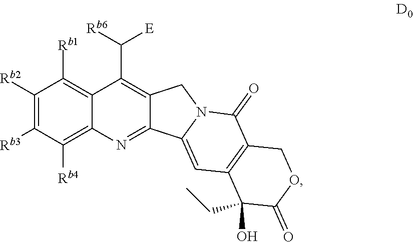

or a salt thereof; wherein
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with R^{a}, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a} —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₆ alkyl-O—C₁-C₆ alkyl-, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, C₁-C₆ hydroxyalkyl-,heteroaryl-, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, C₁-C₆ hydroxyalkyl, —OH, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0
to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
   R^{b6} is H, or is taken together with R^{b1} and the intervening atoms to form a carbocyclo or heterocyclo; and
   R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
   wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

Also provided herein are camptothecin conjugates having a formula:

L-(Q-D)p

or a salt thereof, wherein
L is a Ligand Unit;
the subscript p is an integer of from 1 to 16;
Q is a Linker Unit having a formula selected from the group consisting of:
-Z-A-, -Z-A-RL-; -Z-A-RL-Y-; Z-A-S*-W-; -Z-A-S*-RL-; -Z-A-B(S*)-RL-;
-Z-A-S*-W-RL-, -Z-A-S*-RL-Y-; and -Z-A-B(S*)-RL-Y-;
wherein Z is a Stretcher Unit,
A is a bond or a Connector Unit;
B is a Parallel Connector Unit;
is a Partitioning Agent;
W is a Peptide Unit;
RL is a Releasable Unit;
Y is a Spacer Unit; and
D is a Drug Unit having a formula of

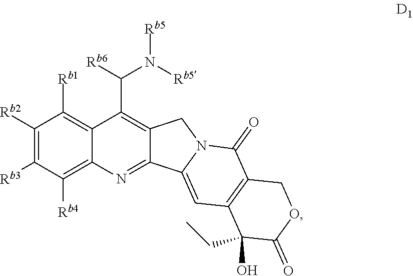

or a salt thereof; wherein
R^{b1} is selected from the group consisting of H, halogen, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a}; R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and R^{a} and R^{a} are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

Also provided herein are Camptothecin Conjugates comprising a Drug Unit corresponding to Formula D₁ or any variation thereof, wherein the nitrogen atom to which R^{b5}′ is bound is replaced by an oxygen atom and R^{b5}′ is absent, such that Q is attached to the Drug Unit via an oxygen atom of the Drug Unit.

In some embodiments of Formula Do, E is —NR^{b5}R^{b5} In some embodiments of Formula Do, E is —OR^{b5}. In some embodiments, E is —OR^{b5}, R^{b1} is H, R^{b2} and R^{b3} combine together with the intervening atoms to form 5-membered heterocyclo, and each of R^{M}, R^{b5}, and R^{b6} are H.

In some embodiments, at least one of R^{b1}, R^{b2}, R^{b3}, and R^{b4} is halogen. In some embodiments, at least one of R^{b1}, R^{b2}, R^{b3}, and R^{b4} is C₁-C₆ alkyl. In some embodiments, at least one of R^{b1}, R^{b2}, R^{b3}, and R^{b4} is —OR^{a}, and R^{a} is H or C₁-C₆ alkyl. In some embodiments, R^{b5} and R^{b5}′ are each H.

In some embodiments, the site of covalent attachment of D to the linker (e.g., secondary linker) of the drug linker moiety is indicated by the dagger in formula D₁ or Dib or any variation thereof (e.g., D₁ₐ-I through D₁ₐ-X, D_{1b}-I through D_{1b}-X, etc.). It is also contemplated that D may be covalently attached to the linker (e.g., secondary linker) of the drug linker moiety at any site in D that is compatible with attachment to the linker (e.g., secondary linker) (e.g., at any OH, NH₂, NHR, NR₂, SH, etc.), whether or not said site is marked by a dagger in any of the formulae herein. In some embodiments, D is connected to the remainder of a Drug-Linker moiety through a OH or NH₂ group of R^{b5}.

In some embodiments, D has a formula selected from the group consisting of

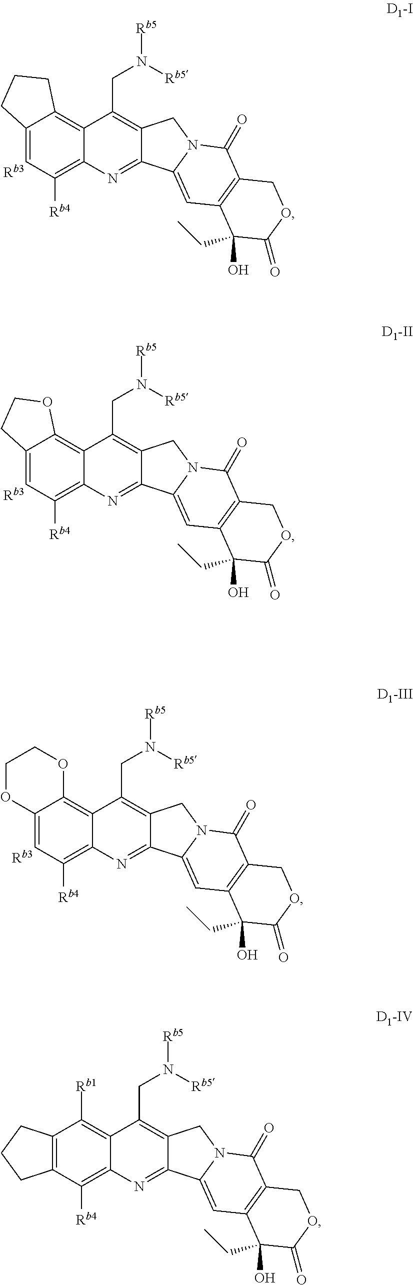

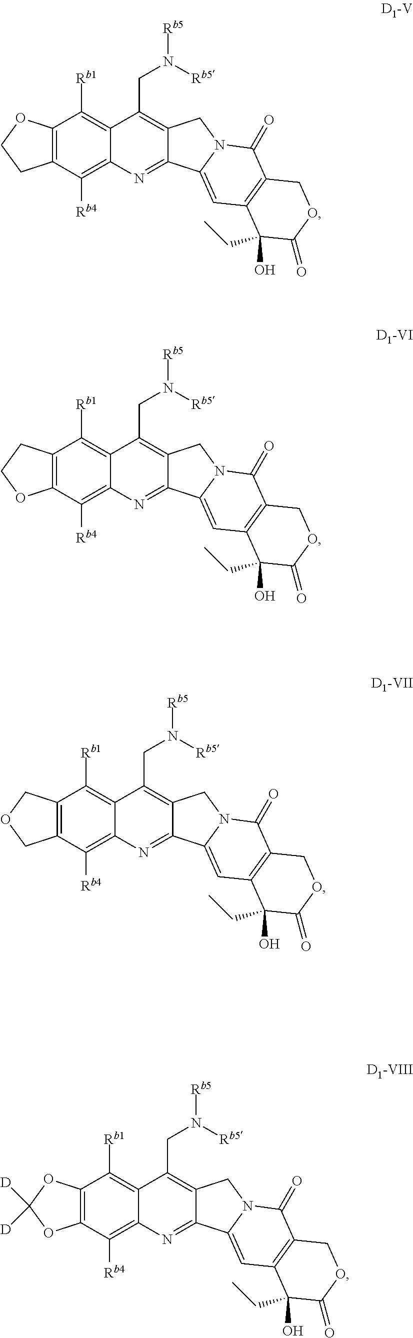

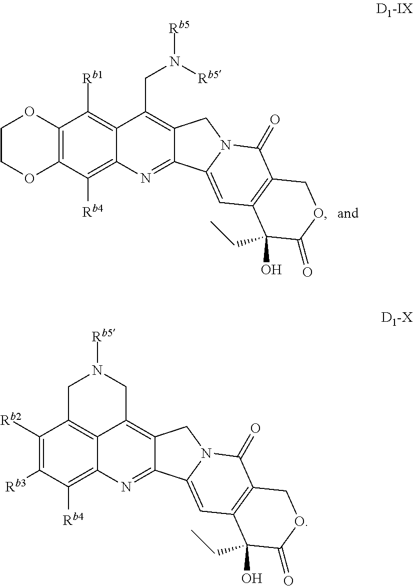

For any of formulas D₁-I through D₁-X and variations thereof, the variables may be defined according to formula Do or any variation thereof, or they may be defined according to formula D₁ or any variation thereof. In some embodiments, D has a structure corresponding to any of formulas D₁-I through D₁-X and variations thereof, wherein the nitrogen atom to which R^{b5}′ is bound is replaced by an oxygen atom and R^{b5}′ is absent.

In some embodiments, D has a formula selected from the group consisting of

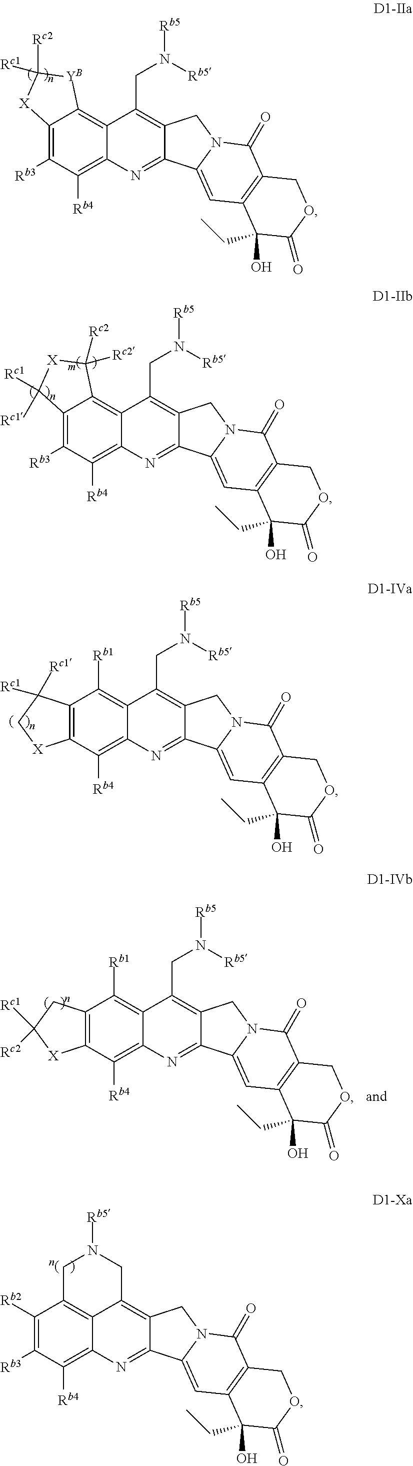

wherein
X and Y^{B} are each independently 0, S, S(O)₂, CR^{x}R^{x}′, or NR^{x}′;
R^{x} and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently
   (i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}′, and —SR^{a}, C₁-C₆ alkyl-C(O)—, C₁-C₆ alkyl-NR^{a}—C(O)—, and C₁-C₆ alkyl-S(O)₂—; or
   (ii) taken together with R^{b1} and the intervening atoms to form a 5- to 7-membered carbocyclo or heterocyclo; or
   (iii) taken together with R^{x}′ and the intervening atoms to form a 3 to 6-membered carbocyclo or heterocyclo;
when m and n are both present, the sum of m+n is 2 or 3; and the remaining variables are as defined for D₁.

In some embodiments, D has a structure corresponding to any of formulas D1-IIa, D1-IIb, D1-IVa, or D1-IVb, wherein the nitrogen atom to which R^{b5}′ is bound is replaced by an oxygen atom and R^{b5}′ is absent.

In some embodiments, D has a formula selected from the group consisting of

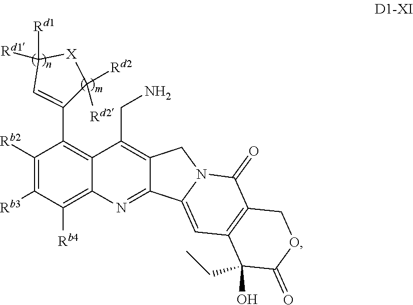

wherein
R^{d1}, R^{d1}′, R^{d2}, and R^{d2}′ are each independently selected from the group consisting of H, halogen, C₁-C₆ alkyl, C₁-C₆ haloalkyl, —OR^{a}, —NR^{a}R^{a}, and —SR^{a}, C₁-C₆ alkyl-C(O)—, C₁-C₆ alkyl-NR^{a}—C(O)—, and C₁-C₆ alkyl-S(O)₂—; and
the remaining variables are as defined for D₁ D1-IIa, D1-IIb, D1-IVa, D1-IVb, and D1-Xa. In some embodiments, D has a formula selected from the group consisting of

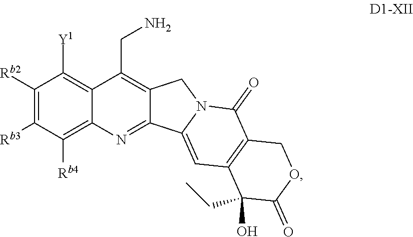

wherein
Y¹ is a 5- or 6-membered heteroaryl, optionally substituted with halogen, C₁-C₆ alkyl, C₁-C₆ haloalkyl, C₁-C₆ hydroxyalkyl, C₁-C₆ aminoalkyl, or C₁-C₆ alkyl-S(O)₂—; and
the remaining variables are as defined for D₁ D1-IIa, D1-Ib, D1-IVa, D1-IVb, and D1-Xa.

In some embodiments, D has a formula selected from the group consisting of

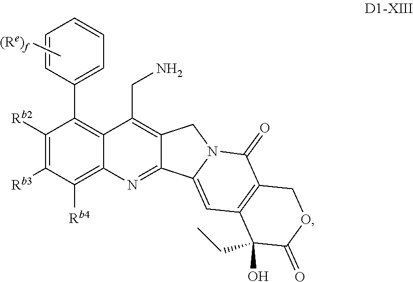

wherein
each R is independently selected from the group consisting of halogen, —OH, —NH₂, C₁-C₆ alkyl, C₁-C₆ haloalkyl, C₁-C₆ hydroxyalkyl, C₁-C₆ alkyl-S(O)₂—, and C₁-C₆ alkyl-NR^{a}—C(O)—;
f is 0, 1,2,3,4, or 5; and
the remaining variables are as defined for D₁ D1-IIa, D1-IIb, D1-IVa, D1-IVb, and D1-Xa.

In some embodiments, D has a formula selected from the group consisting of

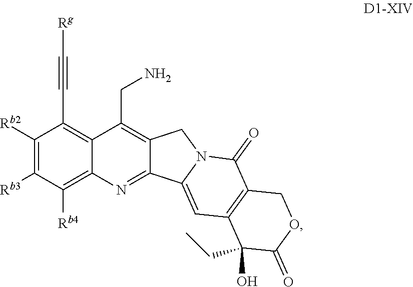

wherein
R⁸ is H, C₁-C₆ alkyl, or 3 to 8-membered heterocyclyl; and
the remaining variables are as defined for D₁ D1-IIa, D1-Ib, D1-IVa, D1-IVb, and D1-Xa.

In some embodiments, R⁸ is C₁-C₆ alkyl.

In some embodiments, D has a formula selected from the group consisting of

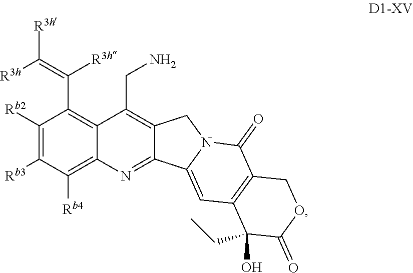

wherein
R^{3h}, R^{3h}′, and R^{3h}″ are each independently selected from the group consisting of H, C₁-C₆ alkyl, C₁-C₆ hydroxyalkyl, C₁-C₆ aminoalkyl, —C(O)—C₁-C₆ alkyl, —C(O)O—C₁-C₆ alkyl, —C(O)NH—C₁-C₆ alkyl, C₆-C₁₀ aryl, —C₆-C₁₀ aryl-C₁-C₆ alkyl, and —C₆-C₁₀ aryl-C₁-C₆ alkoxy;
each optionally substituted with, C₁-C₆ alkyl, C₁-C₆ haloalkyl, —OR^{a}, —NR^{a}R^{a}′, and —SR^{a}; and the remaining variables are as defined for D₁ D1-IIa, D1-Ib, D1-IVa, D1-IVb, and D1-Xa.

In some embodiments, D has a formula selected from the group consisting of

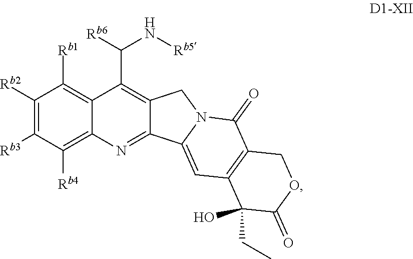

wherein the variables are as defined for D₁ D1-IIa, D1-Ib, D1-IVa, D1-IVb, and D1-Xa.

In some embodiments, D incorporates the structure of a camptothecin having a structure of

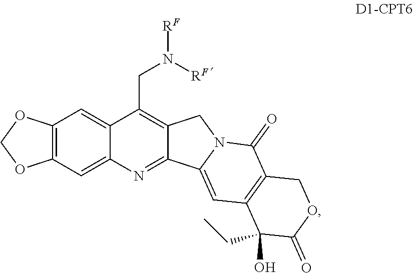

or a pharmaceutically acceptable salt thereof, wherein each R^{F} and R^{F}′ is independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl) -C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆ alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl, NH₂—SO₂-C₁-C₈ alkyl, (C₃-C₁₀ heterocycloalkyl)-C₁_C₄ hydroxyalkyl-, C₁-C₆alkoxy-C(O)—(C₃-C₁₀ heterocycloalkyl)-C₁-C₈ alkyl, phenyl-C(O)—, phenyl-SO₂—, and C₁-C₈ hydroxyalkyl-C₃-C₁₀ hetercycloalkyl, or R^{F} and R^{F}′ are combined with the nitrogen atom to which each is attached to form a 5-, 6- or 7-membered ring having 0 to 3 substituents independently selected from the group consisting of halogen, C₁_C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆alkoxy-C(O)—C₁-C₈ aminoalkyl-, and C₁-C₈ aminoalkyl; wherein the cycloalkyl, heterocycloalkyl, phenyl and heteroaryl portions of R^{F} and R^{F}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂.

In some embodiments, D incorporates the structure of a camptothecin having a structure of

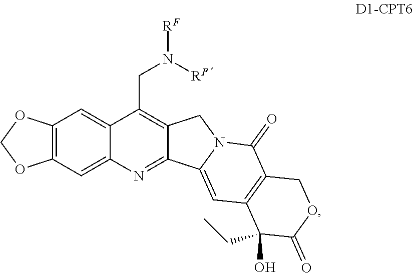

or a pharmaceutically acceptable salt thereof, wherein each R^{F} and R^{F}′ is independently selected from the group consisting of —H, C₁-C₈ alkyl, C₁-C₈ hydroxyalkyl, C₁-C₆-O—C₁-C₆ alkyl, C₁-C₈ aminoalkyl, (C₁-C₄ alkylamino)-C₁-C₈ alkyl-, N,N—(C₁-C₄ hydroxyalkyl) -Ca alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, heteroaryl-C₁-C₆ hydroxyalkyl, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl, NH₂—SO₂-C₁-C₈ alkyl, (C₃-C₁₀ heterocycloalkyl)-C₁-C₄ hydroxyalkyl-, C₁-C₆ alkoxy-C(O)—(C₃-C₁₀ heterocycloalkyl)-C₁-C₈ alkyl, phenyl-C(O)—, phenyl-SO₂—, and C₁-C₈ hydroxyalkyl-C₃-C₁₀ hetercycloalkyl, or R^{F} and R^{F} are combined with the nitrogen atom to which each is attached to form a 5-, 6-, or 7-membered ring having 0 to 3 substituents independently selected from the group consisting of halogen, C₁-C₄ alkyl, —OH, C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)— NH—, C₁-C₆ alkoxy-C(O)—C₁-C₈ aminoalkyl-, and C₁-C₈ aminoalkyl; wherein the cycloalkyl, heterocycloalkyl, phenyl, and heteroaryl portions of R^{F} and R^{F} are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂.

In some embodiments, D has a formula of

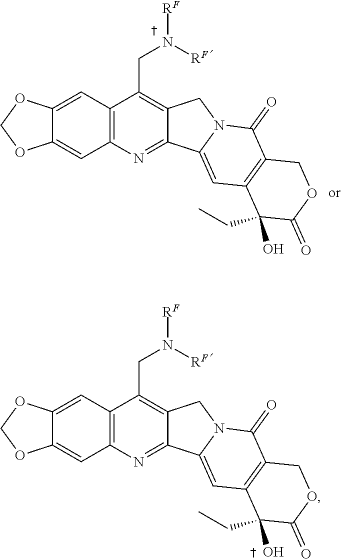

wherein the dagger represents the point of covalent attachment of D to the linker (e.g., secondary linker) of the drug linker moiety. In some embodiments, the dagger denotes attachment of the linker directly to the daggered nitrogen (e.g., by replacement of the R^{b5} moiety). In other embodiments, the dagger denotes attached of the linker to a suitable atom (e.g., a nitrogen or oxygen atom) of the R^{b5} moiety. In some embodiments, R^{F} is selected from the group consisting of C₁-C₆ alkoxy-C(O)—C₁-C₈ aminoalkyl-, C₁-C₆ alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆ alkoxy-C(O)—(C₃-C₁₀ heterocycloalkyl)-C₁-C₈ alkyl-, C₁-C₄ alkyl-SO₂—C₁-C₈ alkyl, NH₂—SO₂—C₁-C₈ alkyl, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl, phenyl-C(O)—, phenyl-SO₂—, and C₁-C₈ hydroxyalkyl-C₃-C₁₀ hetercycloalkyl. In some embodiments, R^{F}′ is —H. In some embodiments, R^{F}′ is methyl. In some embodiments, R^{F} and R^{F} are combined with the nitrogen atom to which each is attached to form a 5-, 6- or 7-membered ring having 0 to 3 substituents independently selected from the group consisting of C₁-C₆alkoxy-C(O)—NH—, C₁-C₆alkoxy-C(O)—C₁-C₈ aminoalkyl-, and C₁-C₈ aminoalkyl. In some embodiments, R^{F} is selected from the group consisting of C₁-C₆ alkyl-O—C₁-C₆ alkyl-, C₁-C₆ alkoxy-C(O)—C₁-C₈ aminoalkyl-, C₁-C₆ alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂—C₁-C₈ alkyl, NH₂—SO₂—C₁-C₈ alkyl, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl, phenyl-C(O)—, phenyl-SO₂—, and C₁-C₈ hydroxyalkyl-C₃-C₁₀ hetercycloalkyl. In some embodiments, R^{F}′ is —H. In some embodiments, R^{F}′ is methyl. In some embodiments, R^{F} and R^{F} are combined with the nitrogen atom to which each is attached to form a 5-, 6- or 7-membered ring having 0 to 3 substituents independently selected from the group consisting of C₁-C₆ alkoxy-C(O)—NH—, C₁-C₆alkoxy-C(O)—C₁-C₈ aminoalkyl-, C₁-C₆ hydroxyalkyl, and C₁-C₈ aminoalkyl.

In some embodiments, D is a Drug Unit having a formula selected from the group consisting of

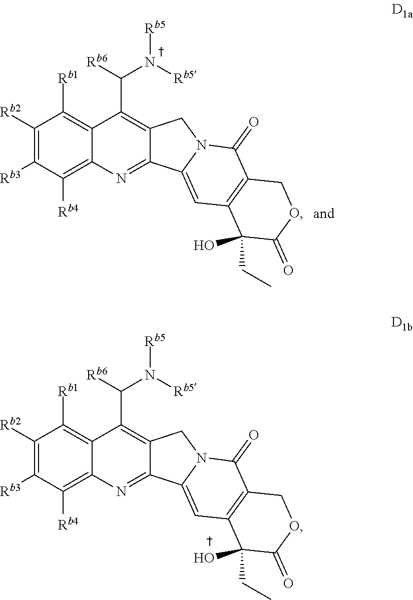

or a salt thereof, wherein the dagger indicates the site of covalent attachment of D to Q and the remaining variables are as defined for D₁. In some embodiments, the remaining variables are as defined for D₀. In some embodiments of Formula D₁ₐ, the dagger denotes attachment of the linker directly to the daggered nitrogen (e.g., by replacement of the R^{b5} moiety). In other embodiments of Formula D₁ₐ, the dagger denotes attached of the linker to a suitable atom (e.g., a nitrogen or oxygen atom) of the R^{b5} moiety.

In some embodiments of Formula D₁ₐ or Formula D_{1b}, R^{b1}, R^{b2}, R^{b3}, and R^{b4} are each hydrogen.

In some embodiments of Formula D₁ₐ or Formula D_{1b}, R^{b1}, R^{b2}, and R^{b4} are hydrogen, and R^{b3} is halogen. In some embodiments, R^{b3} is fluoro.

In some embodiments of Formula D₁ₐ or Formula D_{1b}, R^{b2}, R^{b3}, and R^{b4} are hydrogen, and R^{b3} is halogen. In some embodiments, R^{b1} is fluoro.

In some embodiments of Formula D₁ₐ or Formula D_{1b}, R^{b2} and R^{b4} are hydrogen, and R^{b1} and R^{b3} are both halogen. In some embodiments, R^{b1} and R^{b3} are both fluoro.

In some embodiments of Formula D₁ₐ or Formula D_{1b}, R^{b1} is hydrogen, and R^{b2}, R^{b3} and R^{b4} are each halogen. In some embodiments, R^{b2}, R^{b3}, and R^{b4} are each fluoro.

In some embodiments of Formula D₁ or Formula D_{1b}, R^{b1}, R^{b3}, and R^{b4} are hydrogen, and R^{b2} is C₁-C₆ alkyl, C₁-C₆ haloalkyl, halogen, —OR′ or —SR^{a}. In some embodiments, R^{b2} is C₁-C₆ alkyl or halogen. In some embodiments, R^{b2} is C₁-C₆ alkyl. In some embodiments, R^{b2} is methyl. In some embodiments, R^{b2} is C₁-C₆alkoxy. In some embodiments, R^{b2} is methoxy. In some embodiments, R^{b2} is halogen. In some embodiments, R^{b2} is fluoro. In some embodiments, R^{b2} is chloro. In some embodiments, R^{b2} is bromo. In some embodiments, R^{b2} is C₁-C₆ haloalkyl. In some embodiments, R^{b2} is trifluoromethyl. In some embodiments, R^{b2} is C₁-C₆ haloalkylthio. In some embodiments, R^{b2} is trifluoromethylthio. In some embodiments, R^{b2} is hydroxyl.

In some embodiments of Formula D₁ₐ or Formula D_{1b}, R^{b1} and R^{b4} are hydrogen, R^{b2} is C₁-C₆ alkyl, C₁-C₆ haloalkyl, halogen, —OR^{a}, or —sR^{a}; and R^{b3} is C₁-C₆ alkyl or halogen. In some embodiments, R^{b2} is C₁-C₆ alkyl, C₁-C₆ alkoxy, halogen or hydroxy, and R^{b3} is C₁-C₆ alkyl or halogen. In some embodiments, R^{b2} is C₁-C₆ alkyl. In some embodiments, R^{b2} is methyl. In some embodiments, R^{b2} is C₁-C₆alkoxy. In some embodiments, R^{b2} is halogen. In some embodiments, R^{b2} is fluoro. In some embodiments, R^{b2} is methoxy. In some embodiments, R^{b2} is hydroxyl. In some embodiments, R^{b3} is C₁-C₆ alkyl. In some embodiments, R^{b3} is methyl. In some embodiments, R^{b3} is halogen. In some embodiments, R^{b3} is fluoro. In some embodiments, R^{b2} is C₁-C₆ alkyl and R^{b3} is halogen. In some embodiments, R^{b2} is methyl and R^{b3} is fluoro. In some embodiments, R^{b2} is C₁-C₆ alkoxy and R^{b3} is halogen. In some embodiments, R^{b2} is methoxy and R^{b3} is fluoro. In some embodiments, R^{b2} and R^{b3} are halogen. In some embodiments, R^{b2} and R^{b3} are both fluoro. In some embodiments, R^{b2} is halogen and R^{b3} is C₁-C₆ alkyl. In some embodiments, R^{b2} is fluoro and R^{b3} is methyl. In some embodiments, R^{b2} is hydroxyl and R^{b3} is halogen. In some embodiments, R^{b2} is hydroxyl and R^{b3} is fluoro.

In some embodiments of Formula D₁ₐ or Formula D_{1b}, R^{b2} is C₁-C₆ alkyl, C₁-C₆ haloalkyl, halogen, —OR^{a} or —SR^{a}; both R^{b1} and R^{b3} are independently selected from the group consisting of C₁-C₆ alkyl, halogen, C₂-C₆ alkenyl, (C₆-C₁₂ aryl)-C₂-C₆ alkenyl-optionally substituted with —OR^{a}, and —OR¹⁴; and R^{b4} is hydrogen. In some embodiments, R^{b2} is C₁-C₆ alkyl, C₁-C₆ haloalkyl, halogen, —OR^{a}, or —SR^{a}; both R^{b1} and R^{b3} are independently selected from the group consisting of C₁-C₆ alkyl, halogen, C₂-C₆ alkenyl, (C₆-C₁₂ aryl)-C₂-C₆ alkenyl-, each optionally substituted with —OR^{a}, and —OR^{a}; and R^{b4} is hydrogen. In some embodiments, R^{b1} is C₁-C₆ alkyl. In some embodiments, R^{b1} is methyl. In some embodiments, R^{b1} is halogen. In some embodiments, R^{b1} is fluoro. In some embodiments, R^{b1} is chloro. In some embodiments, R^{b1} is bromo. In some embodiments, R^{b1} is (C₆-C₁₂ aryl)-C₂-C₆ alkenyl-, optionally substituted with —OR^{a}. In some embodiments, R^{b1} is 4-methoxystyryl. In some embodiments, R^{b1} is C₂-C₆ alkenyl. In some embodiments, R^{b1} is vinyl. In some embodiments, R^{b1} is 1-methylvinyl. In some embodiments, R^{b1} is 1-methylvinyl. In some embodiments, R^{b2} is C₁-C₆ alkyl. In some embodiments, R^{b2} is methyl. In some embodiments, R^{b2} is C₁-C₆ alkoxy. In some embodiments, R^{b2} is methoxy. In some embodiments, R^{b2} is hydroxyl. In some embodiments, R^{b3} is C₁-C₆ alkyl. In some embodiments, R^{b3} is methyl. In some embodiments, R^{b3} is ethyl. In some embodiments, R^{b3} is C₁-C₆ alkoxy. In some embodiments, R^{b3} is methoxy. In some embodiments, R^{b3} is halogen. In some embodiments, R^{b3} is fluoro. In some embodiments, R^{b3} is chloro. In some embodiments, R^{b3} is bromo. In some embodiments, R^{b2} is C₁-C₆ alkyl and R^{b1} and R^{b3} are halogen. In some embodiments, R^{b2} is methyl and R^{b1} and R^{b3} are both fluoro. In some embodiments, R^{b2} is methyl, R^{b1} is fluoro and R^{b3} is bromo. In some embodiments, R^{b2} is methyl, R^{b1} is bromo and R^{b3} is fluoro. In some embodiments, R^{b2} is methyl, R^{b1} is chloro and R^{b3} is fluoro. In some embodiments, R^{b2} is methyl, R^{b1} is fluoro and R^{b3} is chloro. In some embodiments, R^{b2} is C₁-C₆ alkoxy and R^{b1} and R^{b3} is halogen. In some embodiments, R^{b2} is methoxy and R^{b1} and R^{b3} are both fluoro. In some embodiments, R^{b2} is methoxy, R^{b1} is bromo and R^{b3} is fluoro. In some embodiments, R^{b2} is methoxy, R^{b1} is fluoro and R^{b3} is bromo. In some embodiments, R^{b2} is hydroxyl and R^{b1} and R^{b3} are halogen. In some embodiments, R^{b2} is hydroxyl and R^{b1} and R^{b3} are both fluoro. In some embodiments, R^{b1} is halogen and R^{b2} and R^{b3} are both C₁-C₆ alkyl. In some embodiments, R^{b1} is fluoro and R^{b2} and R^{b3} are both methyl. In some embodiments, R^{b1} is fluoro, R^{b2} is methyl and R^{b3} is ethyl. In some embodiments, R^{b1} and R^{b2} are both C₁-C₆ alkyl and R^{b3} is halogen. In some embodiments, R^{b1} and R^{b2} are both methyl and R^{b3} is fluoro.

In some embodiments, D has a formula selected from the group consisting of

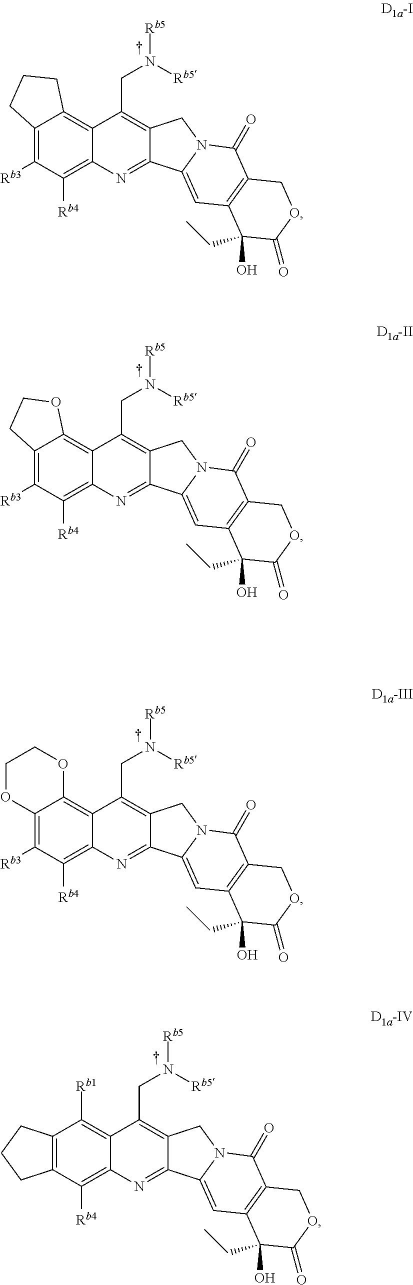

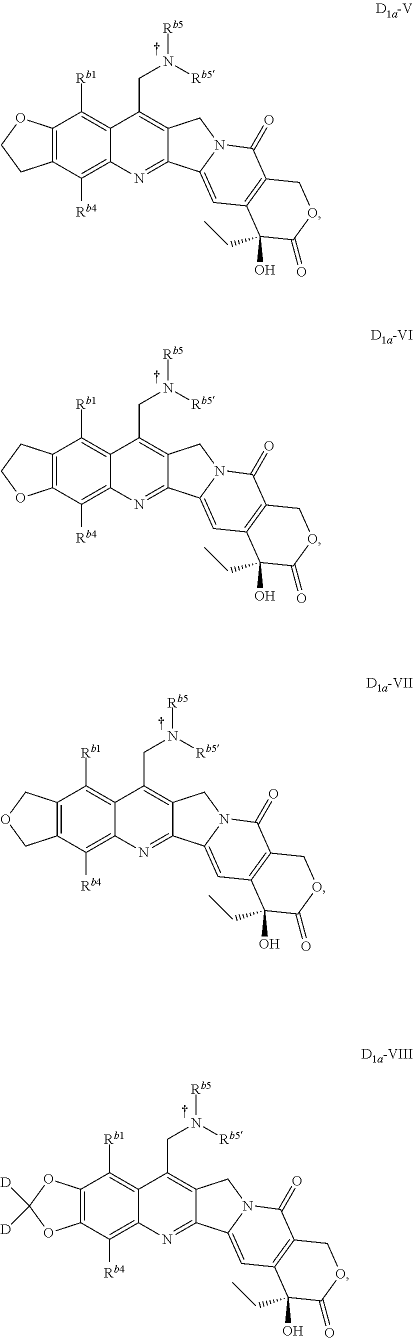

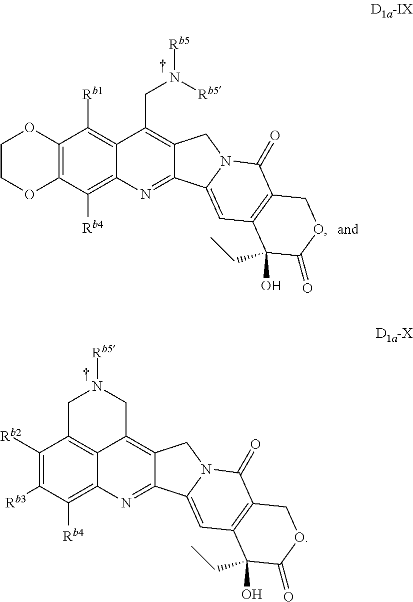

For any of formulas D₁ₐ-I through D₁ₐ-X or any variation thereof, the variables may be defined according to formula D₀ or any variation thereof, or they may be defined according to formula D₁ or any variation thereof.

In some embodiments, D₁ has a formula selected from the group consisting of

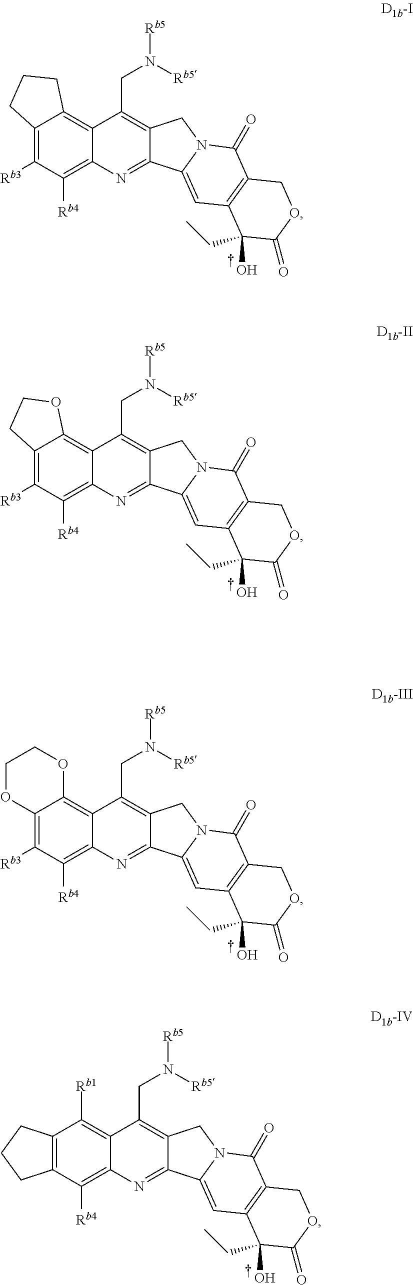

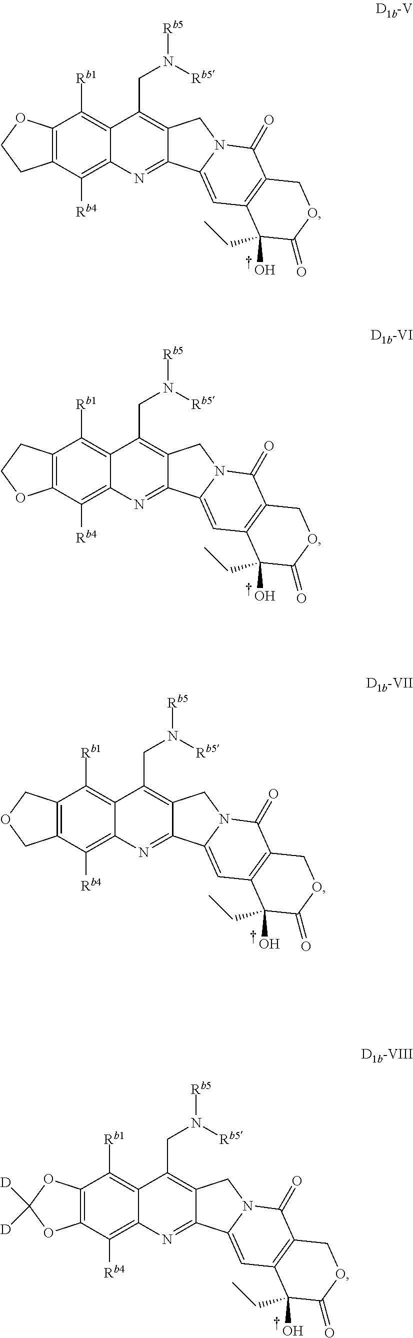

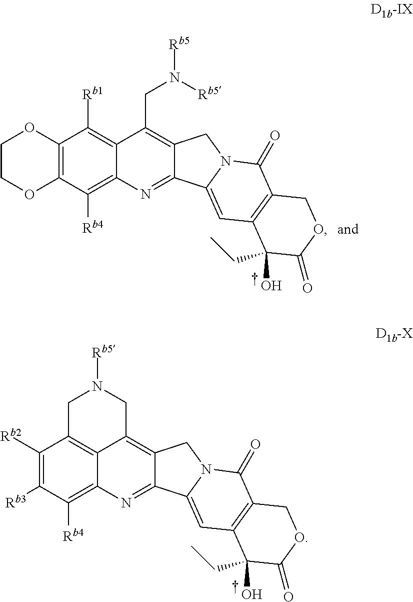

For any of formulas D_{1b}-I through D_{1b}-X or any variation thereof, the variables may be defined according to formula Do or any variation thereof, or they may be defined according to formula D₁ or any variation thereof. In some embodiments, D has a structure corresponding to any of formulas D_{1b}-I through D_{1b}-IX and variations thereof, wherein the nitrogen atom to which R^{b5}′ is bound is replaced by an oxygen atom and R^{b5}′ is absent.

In some embodiments of Formula D₁ₐ or Formula D_{1b}, R^{b1} is combined with R^{b2} and the intervening atoms to form a 5- or 6-membered carbocyclo or heterocyclo ring. In some embodiments, the drug has the structure of Formula D_{1a/b}-I, Formula D_{1a/b}-II, or Formula D_{1a/b}-III as follows:

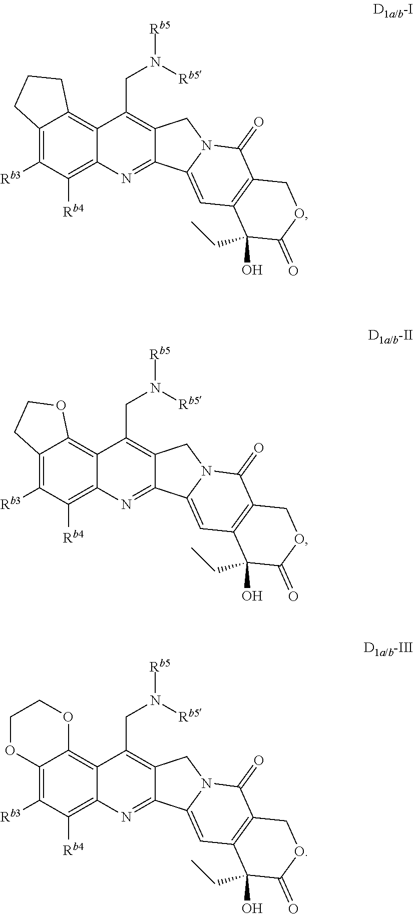

In some embodiments of Formula D₁ₐ or Formula D_{1b}, R^{b2} is combined with R^{b3} and the intervening atoms to form a 5- or 6-membered carbocyclo or heterocyclo ring; wherein one or more hydrogens are optionally replaced with deuterium. In some embodiments, the drug has the structure of Formula D_{1a/b}-IV, D_{1a/b}-V, D_{1a/b}-VI, D_{1a/b}-VII, D_{1a/b}-VIII or D_{1a/b}-IX as follows:

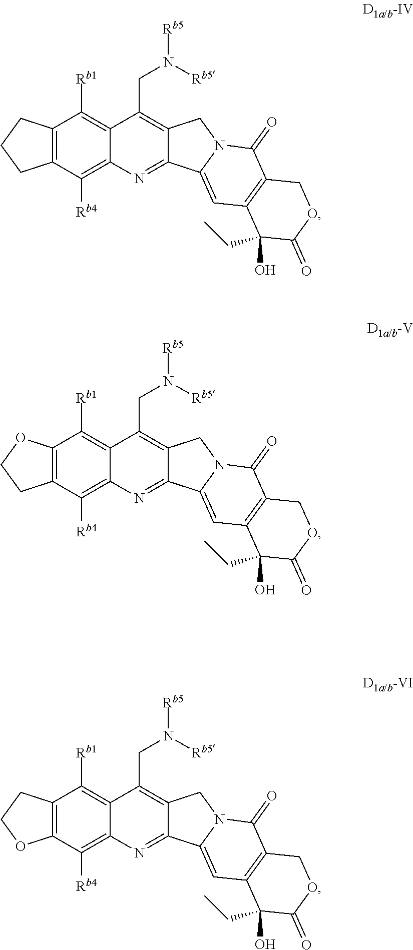

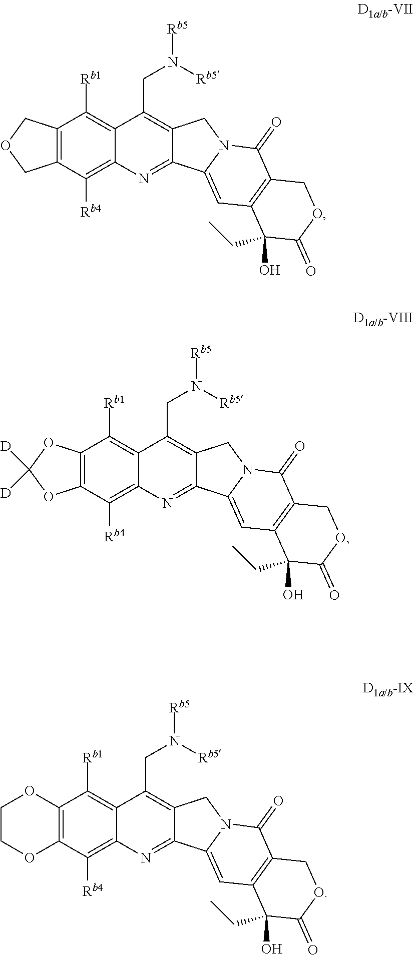

For any of formulas D_{1a/b}-I through D_{1b/a}-IX or any variation thereof, the variables may be defined according to formula D₀ or any variation thereof, or they may be defined according to formula D₁ or any variation thereof. In some embodiments, D has a structure corresponding to any of formulas D_{1a/b}-I through D_{1a/b}-X and variations thereof, wherein the nitrogen atom to which R^{b5} is bound is replaced by an oxygen atom and R^{b5}′ is absent.

In some embodiments of Formula D₁ R^{b5} and R^{b5}′ are both H. In some embodiments, R^{b5} is C₁-C₆ alkyl (e.g., methyl, ethyl) and R^{b5}′ is H.

In some embodiments of Formula D₁ₐ or Formula D_{1b}, R^{b1} is combined with R^{b5} and the intervening atoms to form a 5-, 6-, or 7-membered carbocyclo or heterocyclo ring. In some embodiments, the drug has the structure of Formula D_{1a/b} -X as follows:

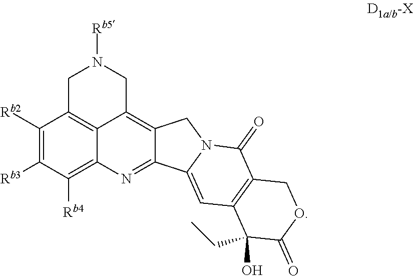

In some embodiments, D has a formula selected from the group consisting of

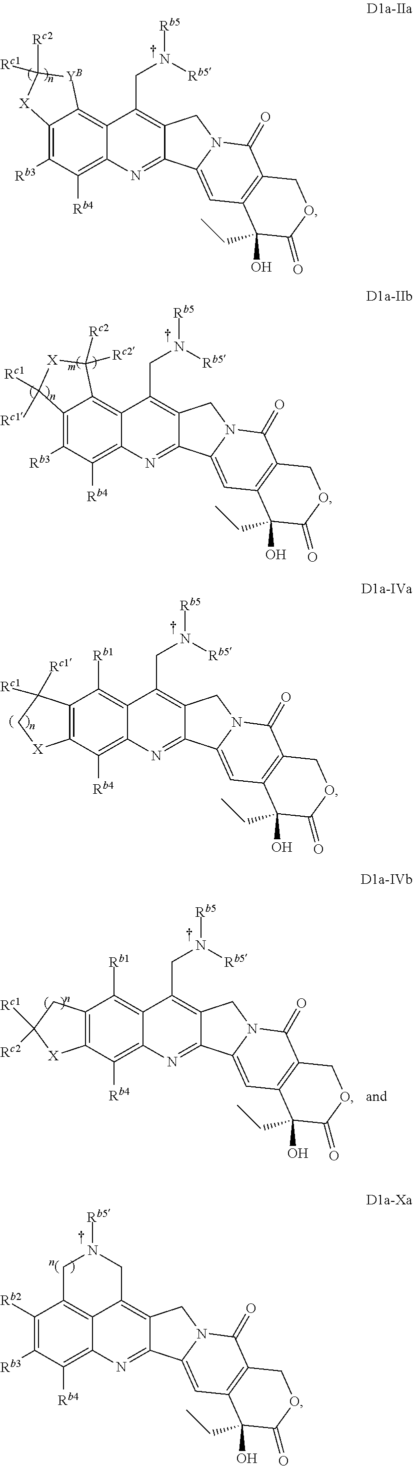

wherein
X and Y^{B} are each independently O, S, S(O)₂, CR^{x}R^{x}′, or NR^{x};
R^{x} and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently
   (i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}, and —SR^{a}, C₁-C₆ alkyl-C(O)—, C₁-C₆ alkyl-NR^{a}—C(O)—, and C₁-C₆ alkyl-S(O)₂—; or
   (ii) taken together with R^{b1} and the intervening atoms to form a 5- to 7-membered carbocyclo or heterocyclo; or
   (iii) taken together with R^{x}′ and the intervening atoms to form a 3- to 6-membered carbocyclo or heterocyclo; or
any two of R^{c1}, R^{c1}′, R^{c2}, and R^{c2}′ are taken together to form a 3- to 6-membered carbocyclo or heterocyclo, and the remaining two of R^{c1}, R¹′, R^{c2}, and R^{c2}′ are independently selected from the group consisting of H, halogen, C₁-C₆ alkyl, C₁-C₆ haloalkyl, —OR^{a}, —NR^{a}R^{a}, and —SR^{a}, —C(O)—C₁-C₆ alkyl, —C(O)NR^{a}—C₁-C₆ alkyl, and —S(O)₂—C₁-C₆ alkyl;
when m and n are both present, the sum of m+n is 2 or 3; and the remaining variables are as defined for D₁ᵤ and D_{1b}.

In some embodiments, D has the formula D1a-IIa, wherein X is O. In some embodiments, D has the formula D1a-IIa, wherein X is S. In some embodiments, D has the formula D1a-IIa, wherein X is CR^{x}R^{x}′. In some embodiments, D has the formula D1a-IIa, wherein Y^{B} is O. In some embodiments, D has the formula D1a-IIa, wherein Y^{B} is S. In some embodiments, D has the formula D1a-IIa, wherein Y^{B} is CR^{x}R^{x}′. In some embodiments, D has the formula D1a-IIa, wherein X is O and Y^{B} is CR^{x}R^{x}′. In some embodiments, D has the formula D1a-IIa, wherein X is O and Y^{B} is CR^{x}R^{x}′, wherein R^{x}′ and R^{x}′ are both H. In some embodiments, D has the formula D1a-IIa, wherein X is CR^{x}R^{x}′; and Y^{B} is O. In some embodiments, D has the formula D1a-IIa, wherein X is CR^{x}R^{x}′; and Y^{B} is O, wherein R^{x} and R^{x}′ are both H. In some embodiments, D has the formula D1a-IIa, wherein X is S and Y^{B} is CR^{x}R^{x}′. In some embodiments, D has the formula D1a-IIa, wherein X is S and Y^{B} is CR^{x}R^{x}′, wherein R^{x} and R^{x}′ are both H. In some embodiments, D has the formula D1a-IIa, wherein X is CR^{x}R^{x}′; and Y^{B} is S. In some embodiments, D has the formula D1a-IIa, wherein X is CR^{x}R^{x}′ and Y^{B} is S, wherein R^{x} and R^{x}′ are both H. In some embodiments, D has the formula D1a-IIa, wherein X and Y^{B} are both CR^{x}′. In some embodiments, D has the formula D1a-IIa, wherein X and Y^{B} are both CR^{x}′, wherein R^{x} and R^{x}′ are both H. In some embodiments, D has the formula D1a-IIa, wherein X and Y^{B} are both CR^{x}R^{x}′, and R^{b3} is halo. In some embodiments, D has the formula D1a-IIa, wherein X and Y^{B} are both CR^{x}R^{x}′, R^{x} and R^{x}′ are both H, and R^{b3} is halo. In some embodiments, D has the formula D1a-IIa, wherein X and Y^{B} are both CR^{x}R^{x}′, R^{x} and R^{x}′ are both H, and R^{b3} is fluoro. In some embodiments, D has the formula D1a-IIa, wherein X and Y^{B} are both CR^{x}′, wherein R^{x} and R^{x}′ are both H. In some embodiments, n is 1 or 2. In some embodiments, n is 1. In some embodiments, n is 2. In some embodiments, R^{b5} is H. In some embodiments, R^{b5} is H. In some embodiments, R^{b5} and R^{b5}′ are both H.

In some embodiments, D has the formula D1a-IIb, wherein X is O. In some embodiments, D has the formula D1a-IIb, wherein X is CR^{x}R^{x}′. In some embodiments, D has the formula D1a-IIb, wherein X is CR^{x}R^{x}′, wherein R^{x} and R^{x}′ are both H. In some embodiments, D has the formula D1a-IIb, wherein X is CR^{x}R^{x}′, and R^{b3} is halo. In some embodiments, D has the formula D1a-IIb, wherein X is CR^{x}R^{x}′, R^{x} and R^{x}′ are both H, and R^{b3} is halo. In some embodiments, D has the formula D1a-IIb, wherein X is CR^{x}R^{x}′, R^{x}′ and R^{x}′ are both H, and R^{b3} is fluoro. In some embodiments, n is 1. In some embodiments, m is 1. In some embodiments, n and m are both 1. In some embodiments, R^{b5} is H. In some embodiments, R^{b5}′ is H. In some embodiments, R^{b5} and R^{b5}′ are both H.

In some embodiments, D has the formula D1a-IVa, wherein X is O. In some embodiments, D has the formula D1a-IVa, wherein X is S. In some embodiments, D has the formula D1a-IVa, wherein X is CR^{x}R^{x}′. In some embodiments, D has the formula D1a-IVa, wherein X is CR^{x}R^{x}′, wherein R^{x} and R^{x}′ are both H. In some embodiments, D has the formula D1a-IVa, wherein X is CR^{x}R^{x}′ and R^{b1} is halo. In some embodiments, D has the formula D1a-IVa, wherein X is CR^{x}R^{x}′, R^{x} and R^{x}′ are both H, and R^{b1} is halo. In some embodiments, D has the formula D1a-IVa, wherein X is CR^{x}R^{x}′, R^{x} and R^{x}′ are both H, and R^{b1} is fluoro. In some embodiments, D has the formula D1a-IVa, wherein X is O and R^{c1} is C₁-C₆ alkyl. In some embodiments, D has the formula D1a-IVa, wherein X is O and R^{c1} is methyl. In some embodiments, n is 1. In some embodiments, n and m are both 1. In some embodiments, R^{b5} is H. In some embodiments, R^{b5}′ is H. In some embodiments, R^{b5} and R^{b5}′ are both H.

In some embodiments, D has the formula D1a-IVb, wherein X is O. In some embodiments, D has the formula D1a-IVb, wherein X is S. In some embodiments, D has the formula D1a-IVb, wherein X is CR^{x}′. In some embodiments, D has the formula D1a-IVb, wherein X is CR^{x}′, wherein R^{x} and R^{x}′ are both H. In some embodiments, D has the formula D1a-IVb, wherein X is CR^{x}R^{x}′; and R^{b1} is halo. In some embodiments, D has the formula D1a-IVb, wherein X is CR^{x}′, R^{x} and R^{x}′ are both H, and R^{b1} is halo. In some embodiments, D has the formula D1a-IVb, wherein X is CR^{x}′, R^{x} and R^{x}′ are both H, and R^{b1} is fluoro. In some embodiments, D has the formula D1a-IVb, wherein X is O and R^{c1} is C₁-C₆ alkyl. In some embodiments, D has the formula D1a-IVb, wherein X is O and R^{c1} is methyl. In some embodiments, n is 1. In some embodiments, n and m are both 1. In some embodiments, R^{b5} is H. In some embodiments, R^{b5}′ is H. In some embodiments, R^{b5} and R^{b5}′ are both H.

In some embodiments, D has the formula D1a-Xa, wherein n is 1 or 2. In some embodiments, D has the formula D1a-Xa, wherein n is 1. In some embodiments, D has the formula D1a-Xa, wherein n is 2. In some embodiments, D has the formula D1a-Xa, wherein R^{b5}′ is H. In some embodiments, D has the formula D1a-Xa, wherein n is 1 and R^{b5}′ is H. In some embodiments, R^{b2} is OH. In some embodiments, R^{b3} is halo. In some embodiments, R^{b3} is fluoro. In some embodiments, R^{b2} is OH and R^{b3} is fluoro.

In some embodiments, D has a formula selected from the group consisting of

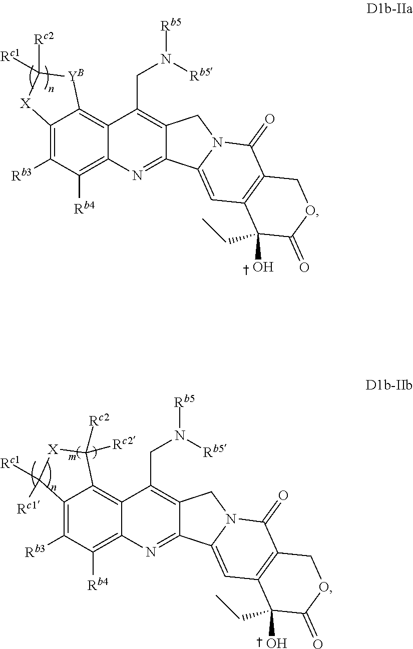

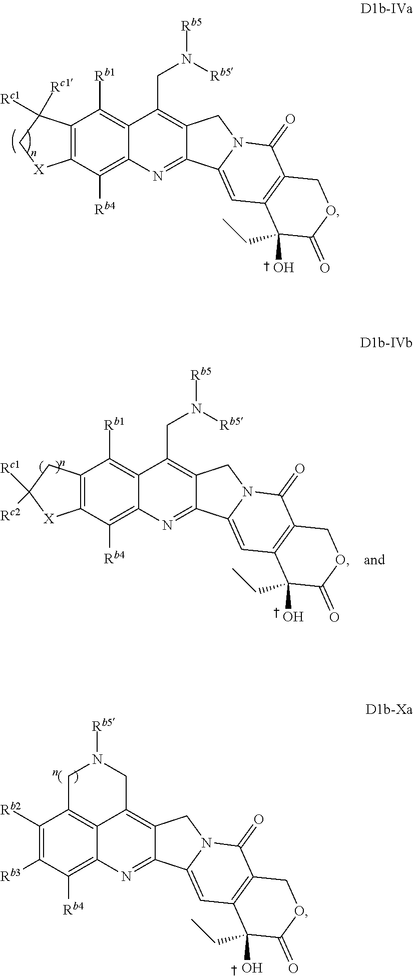

wherein the variables are as defined for D₁ₐ, D_{1b}, D1a-IIa, D1a-IIb, D1a-IVa, D1a-IVb, and D1a-Xa. In some embodiments, D has a structure corresponding to any of formulas D₁ₐ, D_{1b}, D1a-IIa, D1a-IM, D1a-IVa, and D1a-IVb and variations thereof, wherein the nitrogen atom to which R^{b5}′ is bound is replaced by an oxygen atom and R^{b5}′ is absent.

In some embodiments, D has the formula D1b-IIa, wherein X is O. In some embodiments, D has the formula D1b-IIa, wherein X is S. In some embodiments, D has the formula D1b-IIa, wherein X is CR^{x}R^{x}′. In some embodiments, D has the formula D1b-IIa, wherein Y^{B} is 0. In some embodiments, D has the formula D1b-IIa, wherein Y^{B} is S. In some embodiments, D has the formula D1b-IIa, wherein Y^{B} is CR^{x}R^{x}′. In some embodiments, D has the formula D1b-IIa, wherein X is O and Y^{B} is CR^{x}R^{x}′. In some embodiments, D has the formula D1b-IIa, wherein X is O and Y^{B} is CR^{x}R^{x}′, wherein R^{x}′ and R^{x}′ are both H. In some embodiments, D has the formula D1b-IIa, wherein X is CR^{x}R^{x}′; and Y^{B} is 0. In some embodiments, D has the formula D1b-IIa, wherein X is CR^{x}R^{x}′ and Y^{B} is 0, wherein R^{x} and R^{x}′ are both H. In some embodiments, D has the formula D1b-IIa, wherein X is S and Y^{B} is CR^{x}R^{x}′. In some embodiments, D has the formula D1b-IIa, wherein X is S and Y^{B} is CR^{x}R^{x}′, wherein R^{x} and R^{x}′ are both H. In some embodiments, D has the formula D1b-IIa, wherein X is CR^{x}R^{x}′; and Y^{B} is S. In some embodiments, D has the formula D1b-IIa, wherein X is CR^{x}R^{x}′ and Y^{B} is S, wherein R^{x} and R^{x}′ are both H. In some embodiments, D has the formula D1b-IIa, wherein X and Y^{B} are both CR^{x}′. In some embodiments, D has the formula D1b-IIa, wherein X and Y^{B} are both CR^{x}′, wherein R^{x} and R^{x}′ are both H. In some embodiments, D has the formula D1b-IIa, wherein X and Y^{B} are both CR^{x}R^{x}′, and R^{b3} is halo. In some embodiments, D has the formula D1b-IIa, wherein X and Y^{B} are both CR^{x}R^{x}′, R^{x} and R^{x}′ are both H, and R^{b3} is halo. In some embodiments, D has the formula D1b-IIa, wherein X and Y^{B} are both CR^{x}R^{x}′, R^{x} and R^{x}′ are both H, and R^{b3} is fluoro. In some embodiments, D has the formula D1b-IIa, wherein X and Y^{B} are both CR^{x}′, wherein R^{x} and R^{x}′ are both H. In some embodiments, n is 1 or 2. In some embodiments, n is 1. In some embodiments, n is 2. In some embodiments, R^{b5} is H. In some embodiments, R^{b5} is H. In some embodiments, R^{b5} and R^{b5}′ are both H.

In some embodiments, D has the formula D1b-IIb, wherein X is O. In some embodiments, D has the formula D1b-IM, wherein X is CR^{x}R^{x}′. In some embodiments, D has the formula D1b-IIb, wherein X is CR^{x}′, wherein R^{x} and R^{x}′ are both H. In some embodiments, D has the formula D1b-IM, wherein X is CR^{x}R^{x}′, and R^{b3} is halo. In some embodiments, D has the formula D1b-IM, wherein X is CR^{x}R^{x}′, R^{x}′ and R^{x} are both H, and R^{b3} is halo. In some embodiments, D has the formula D1b-IM, wherein X is CR^{x}R^{x}′, R^{x}′ and R^{x}′ are both H, and R^{b3} is fluoro. In some embodiments, n is 1. In some embodiments, m is 1. In some embodiments, n and m are both 1. In some embodiments, R^{b5} is H. In some embodiments, R^{b5}′ is H. In some embodiments, R^{b5} and R^{b5}′ are both H.

In some embodiments, D has the formula D1b-IVa, wherein X is O. In some embodiments, D has the formula D1b-IVa, wherein X is S. In some embodiments, D has the formula D1b-IVa, wherein X is CR^{x}′. In some embodiments, D has the formula D1b-IVa, wherein X is CR^{x}′, wherein R^{x} and R^{x}′ are both H. In some embodiments, D has the formula D1b-IVa, wherein X is CR^{x}R^{x}′; and R^{b1} is halo. In some embodiments, D has the formula D1b-IVa, wherein X is CR^{x}′, R^{x} and R^{x}′ are both H, and R^{b1} is halo. In some embodiments, D has the formula D1b-IVa, wherein X is CR^{x}′, R^{x} and R^{x}′ are both H, and R^{b1} is fluoro. In some embodiments, D has the formula D1b-IVa, wherein X is O and R^{c1} is C₁-C₆ alkyl. In some embodiments, D has the formula D1b-IVa, wherein X is O and R^{c1} is methyl. In some embodiments, n is 1. In some embodiments, n and m are both 1. In some embodiments, R^{b5} is H. In some embodiments, R^{b5}′ is H. In some embodiments, R^{b5} and R^{b5}′ are both H.

In some embodiments, D has the formula D1b-IVb, wherein X is O. In some embodiments, D has the formula D1b-IVb, wherein X is S. In some embodiments, D has the formula D1b-IVb, wherein X is One. In some embodiments, D has the formula D1b-IVb, wherein X is CR^{x}R^{x}′, wherein R^{x} and R^{x}′ are both H. In some embodiments, D has the formula D1b-IVb, wherein X is CR^{x}′ R^{x}′ and R^{b1} is halo. In some embodiments, D has the formula D1b-IVb, wherein X is CR^{x}R^{x}′, R^{x} and R^{x}′ are both H, and R^{b1} is halo. In some embodiments, D has the formula D1b-IVb, wherein X is CR^{x}R^{x}′, R^{x} and R^{x}′ are both H, and R^{b1} is fluoro. In some embodiments, D has the formula D1b-IVb, wherein X is O and R^{c1} is C₁-C₆ alkyl. In some embodiments, D has the formula D1b-IVb, wherein X is O and R^{c1} is methyl. In some embodiments, n is 1. In some embodiments, n and m are both 1. In some embodiments, R^{b5} is H. In some embodiments, R^{b5} is H. In some embodiments, R^{b5} and R^{b5}′ are both H.

In some embodiments, D has the formula D1b-Xa, wherein n is 1 or 2. In some embodiments, D has the formula D1b-Xa, wherein n is 1. In some embodiments, D has the formula D1b-Xa, wherein n is 2. In some embodiments, D has the formula D1b-Xa, wherein R^{b5}′ is H. In some embodiments, D has the formula D1b-Xa, wherein n is 1 and R^{b5}′ is H. In some embodiments, R^{b2} is OH. In some embodiments, R^{b3} is halo. In some embodiments, R^{b3} is fluoro. In some embodiments, R^{b2} is OH and R^{b3} is fluoro.

In some embodiments, D has a formula selected from the group consisting of wherein

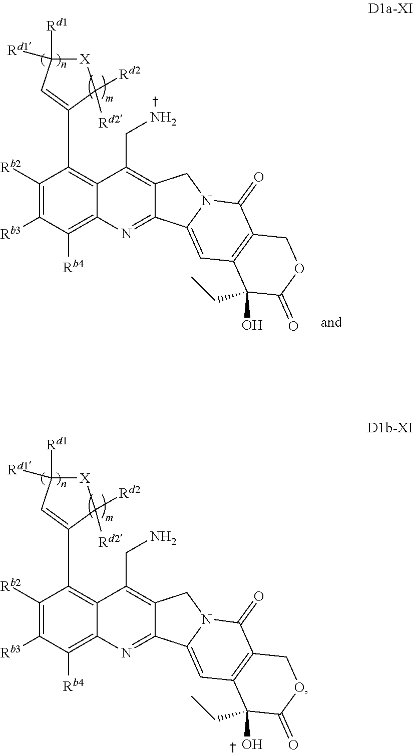

R^{d1}, R^{d1}′, R^{d2}, and R^{d2}′ are each independently selected from the group consisting of H, halogen, C₁-C₆ alkyl, C₁-C₆ haloalkyl, —OR^{a}, —NR^{a}R^{a}, and —SR^{a}, —C(O)—C₁-C₆ alkyl, —C(O)NR^{a}—C₁-C₆ alkyl, and —S(O)₂—C₁-C₆ alkyl; and
the remaining variables are as defined for D₁ₐ and D_{1b}. In some embodiments, D has a structure corresponding to any of formulas D1a-XI and D1b-XI and variations thereof, wherein the NH₂ group is replaced by an OH group.

In some embodiments, D has the formula D1a-XI, wherein R^{b2} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1a-XI, wherein R^{b3} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1a-XI, wherein X is O, S, S(O)₂, CR^{x}R^{x}′, or NR^{x}; wherein R^{x} and R^{x}′ are each independently selected from the group consisting of H, OH, C₁-C₆ alkyl, —C(O)—C₁-C₆ alkyl, —C(O)NH—C₁-C₆ alkyl, and —S(O)₂—C₁-C₆ alkyl.

In some embodiments, D has the formula D1a-XI, wherein R^{b2} is C₁-C₆ alkyl. In some embodiments, D has the formula D1a-XI, wherein R^{b2} is methyl. In some embodiments, D has the formula D1a-XI, wherein R^{b3} is halo. In some embodiments, D has the formula D1a-XI, wherein R^{b3} is fluoro. In some embodiments, D has the formula D1a-XI, wherein R^{b2} is methyl and R^{b3} is fluoro. In some embodiments, D has the formula D1a-XI, wherein n is 1 or 2. In some embodiments, D has the formula D1a-XI, wherein n is 1. In some embodiments, D has the formula D1a-XI, wherein n is 2. In some embodiments, D has the formula D1a-XI, wherein m is 1 or 2. In some embodiments, D has the formula D1a-XI, wherein m is 1. In some embodiments, D has the formula D1a-XI, wherein m is 2. In some embodiments, D has the formula D1a-XI, wherein n and m are both 1. In some embodiments, D has the formula D1a-XI, wherein n is 1 and m is 2. In some embodiments, D has the formula D1a-XI, wherein n is 2 and m is 1. In some embodiments, D has the formula D1a-XI, wherein X is O. In some embodiments, D has the formula D1a-XI, wherein X is CR^{x}R^{x}′. In some embodiments, D has the formula D1a-XI, wherein X is CR^{x}R^{x}′, and R^{x} and R^{x}′ are both H. In some embodiments, D has the formula D1a-XI, wherein X is NR^{x}. In some embodiments, D has the formula D1a-XI, wherein X is NR^{x}, wherein R^{x}′ is C₁-C₆ alkyl. In some embodiments, D has the formula D1a-XI, wherein X is NR^{x}, wherein R^{x}′ is methyl. In some embodiments, D has the formula D1a-XI, wherein X is NR^{x}, wherein R^{x}′ is methyl. In some embodiments, D has the formula D1a-XI, wherein X is S. In some embodiments, D has the formula D1a-XI, wherein X is S(O)₂. In some embodiments, D has the formula D1a-XI, wherein X is —S(O)₂—C₁-C₆ alkyl. In some embodiments, D has the formula D1a-XI, wherein X is —S(O)₂—CH₃.

In some embodiments, D has the formula D1b-XI, wherein R^{b2} is selected from the group consisting of H, halogen, C₁-C₆ alkyl, C₁-C₆ haloalkyl, —OR^{a}, —NHR^{a}, and —SR^{a}, wherein each IV is independently selected from the group consisting of H, C₁-C₆ alkyl, and C₁-C₆ haloalkyl. In some embodiments, D has the formula D1b-XI, wherein R^{b3} is selected from the group consisting of H, halogen, C₁-C₆ alkyl, C₁-C₆ haloalkyl, —OR^{a}, —NHR^{a}, and —SR^{a}, wherein each IV is independently selected from the group consisting of H, C₁-C₆ alkyl, and C₁-C₆ haloalkyl. In some embodiments, D has the formula D1b-XI, wherein X is O, S, S(O)₂, CR^{x}R^{x}′, or NR^{x}; wherein R^{x} and R^{x}′ are each independently selected from the group consisting of H, OH, C₁-C₆ alkyl, —C(O)—C₁-C₆ alkyl, —C(O)NH—C₁-C₆ alkyl, and —S(O)₂—C₁-C₆ alkyl.

In some embodiments, D has the formula D1b-XI, wherein R^{b2} is C₁-C₆ alkyl. In some embodiments, D has the formula D1b-XI, wherein R^{b2} is methyl. In some embodiments, D has the formula D1b-XI, wherein R^{b3} is halo. In some embodiments, D has the formula D1b-XI, wherein R^{b3} is fluoro. In some embodiments, D has the formula D1b-XI, wherein R^{b2} is methyl and R^{b3} is fluoro. In some embodiments, D has the formula D1b-XI, wherein n is 1 or 2. In some embodiments, D has the formula D1b-XI, wherein n is 1. In some embodiments, D has the formula D1b-XI, wherein n is 2. In some embodiments, D has the formula D1b-XI, wherein m is 1 or 2. In some embodiments, D has the formula D1b-XI, wherein m is 1. In some embodiments, D has the formula D1b-XI, wherein m is 2. In some embodiments, D has the formula D1b-XI, wherein n and m are both 1. In some embodiments, D has the formula D1b-XI, wherein n is 1 and m is 2. In some embodiments, D has the formula D1b-XI, wherein n is 2 and m is 1. In some embodiments, D has the formula D1b-XI, wherein X is O. In some embodiments, D has the formula D1b-XI, wherein X is CR^{x}′ R^{x}′ ^{e}. In some embodiments, D has the formula D1b-XI, wherein X is CR^{x}R^{x}′, and R^{x} and R^{x}′ are both H. In some embodiments, D has the formula D1b-XI, wherein X is NR^{x}. In some embodiments, D has the formula D1b-XI, wherein X is NR^{x}, wherein R^{x}′ is C₁-C₆ alkyl. In some embodiments, D has the formula D1b-XI, wherein X is NR^{x}, wherein R^{x}′ is methyl. In some embodiments, D has the formula D1b-XI, wherein X is NR^{x}, wherein R^{x}′ is methyl. In some embodiments, D has the formula D1b-XI, wherein X is S. In some embodiments, D has the formula D1b-XI, wherein X is S(O)₂. In some embodiments, D has the formula D1b-XI, wherein X is —S(O)₂—C₁-C₆ alkyl. In some embodiments, D has the formula D1b-XI, wherein X is —S(O)₂—CH₃.

In some embodiments, D has a formula selected from the group consisting of

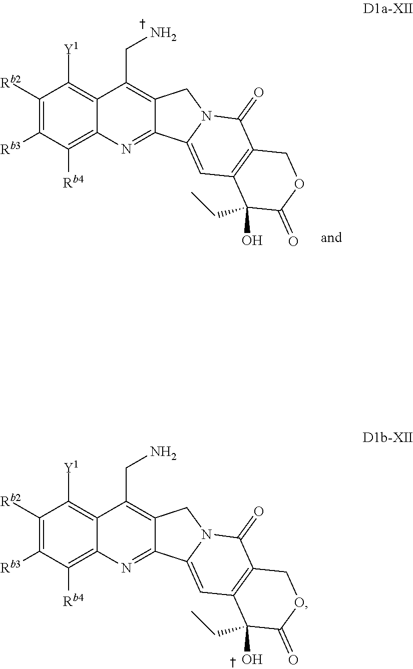

wherein
Y¹ is a 5- or 6-membered heteroaryl, optionally substituted with halogen, C₁-C₆ alkyl, C₁-C₆ haloalkyl, C₁-C₆ hydroxyalkyl, C₁-C₆ aminoalkyl, or C₁-C₆ alkyl-S(O)₂—; and the remaining variables are as defined for D₁ₐ and D_{1b}. In some embodiments, D has a structure corresponding to any of formulas D1a-XII and D1b-XII and variations thereof, wherein the NH₂ group is replaced by an OH group.

In some embodiments, D has the formula D1a-XII, wherein R^{b2} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1a-XII, wherein R^{b3} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl.

In some embodiments, D has the formula D1a-XII, wherein R^{b2} is C₁-C₆ alkyl. In some embodiments, D has the formula D1a-XII, wherein R^{b2} is methyl. In some embodiments, D has the formula D1a-XII, wherein R^{b3} is halo. In some embodiments, D has the formula D1a-XII, wherein R^{b3} is fluoro. In some embodiments, D has the formula D1b-XI, wherein R^{b2} is methyl and R^{b3} is fluoro. In some embodiments, D has the formula D1a-XII, wherein Y¹ is a 5-membered heteroaryl optionally substituted with C₁-C₆ alkyl, C₁-C₆ hydroxyalkyl, or —S(O)₂—C₁-C₆ alkyl. In some embodiments, D has the formula D1a-XII, wherein Y¹ is an unsubstituted 5-membered heteroaryl. In some embodiments, D has the formula D1a-XII, wherein Y¹ is an unsubstituted thiophene. In some embodiments, D has the formula D1a-XII, wherein Y¹ is an unsubstituted thiophene; and R^{b2} is methyl and R^{b3} is fluoro. In some embodiments, D has the formula D1a-XII, wherein Y¹ is a 5-membered heteroaryl, substituted with C₁-C₆ alkyl, C₁-C₆ hydroxyalkyl, or —S(O)₂—C₁-C₆ alkyl. In some embodiments, D has the formula D1a-XII, wherein Y¹ is a thiophene, substituted with C₁-C₆ alkyl, C₁-C₆ hydroxyalkyl, or —S(O)₂—C₁-C₆ alkyl. In some embodiments, D has the formula D1a-XII, wherein Y¹ is a thiophene, substituted with C₁-C₆ hydroxyalkyl. In some embodiments, D has the formula D1a-XII, wherein Y¹ is a thiophene, substituted with hydroxyethyl. In some embodiments, D has the formula D1a-XII, wherein Y¹ is a thiophene, substituted with hydroxyethyl; and R^{b2} is methyl and R^{b3} is fluoro. In some embodiments, D has the formula D1a-XII, wherein Y¹ is a furan. In some embodiments, D has the formula D1a-XII, wherein Y¹ is an unsubstituted furan. In some embodiments, D has the formula D1a-XII, wherein Y¹ is a pyrrole. In some embodiments, D has the formula D1a-XII, wherein Y¹ is a substituted pyrrole. In some embodiments, D has the formula D1a-XII, wherein Y¹ is a pyrrole substituted by —S(O)₂—C₁-C₆ alkyl. In some embodiments, D has the formula D1a-XII, wherein Y¹ is a pyrrole substituted by —S(O)₂—CH₃. In some embodiments, D has the formula D1a-XII, wherein Y¹ is a pyridine. In some embodiments, D has the formula D1a-XII, wherein Y¹ is an unsubstituted pyridine. In some embodiments, D has the formula D1a-XII, wherein Y¹ is an isoxazole. In some embodiments, D has the formula D1a-XII, wherein Y¹ is an unsubstituted isoxazole. In some embodiments, D has the formula D1a-XII, wherein Y¹ is an isoxazole substituted by one or more C₁-C₆ alkyl. In some embodiments, D has the formula D1a-XII, wherein Y¹ is an isoxazole substituted by one or more methyl. In some embodiments, D has the formula D1a-XII, wherein Y¹ is an isoxazole substituted by one methyl group. In some embodiments, D has the formula D1a-XII, wherein Y¹ is an isoxazole substituted by two methyl groups.

In some embodiments, D has the formula D1b-XII, wherein R^{b2} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1b-XII, wherein R^{b3} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl.

In some embodiments, D has the formula D1b-XII, wherein R^{b2} is C₁-C₆ alkyl. In some embodiments, D has the formula D1b-XII, wherein R^{b2} is methyl. In some embodiments, D has the formula D1b-XII, wherein R^{b3} is halo. In some embodiments, D has the formula D1b-XII, wherein R^{b3} is fluoro. In some embodiments, D has the formula D1b-XI, wherein R^{b2} is methyl and R^{b3} is fluoro. In some embodiments, D has the formula D1b-XII, wherein Y¹ is a 5-membered heteroaryl optionally substituted with C₁-C₆ alkyl, C₁-C₆ hydroxyalkyl, or —S(O)₂—C₁-C₆ alkyl. In some embodiments, D has the formula D1b-XII, wherein Y¹ is an unsubstituted 5-membered heteroaryl. In some embodiments, D has the formula D1b-XII, wherein Y¹ is an unsubstituted thiophene. In some embodiments, D has the formula D1b-XII, wherein Y¹ is an unsubstituted thiophene; and R^{b2} is methyl and R^{b3} is fluoro. In some embodiments, D has the formula D1b-XII, wherein Y¹ is a 5-membered heteroaryl, substituted with C₁-C₆ alkyl, C₁-C₆ hydroxyalkyl, or —S(O)₂—C₁-C₆ alkyl. In some embodiments, D has the formula D1b-XII, wherein Y¹ is a thiophene, substituted with C₁-C₆ alkyl, C₁-C₆ hydroxyalkyl, or —S(O)₂—C₁-C₆ alkyl. In some embodiments, D has the formula D1b-XII, wherein Y¹ is a thiophene, substituted with C₁-C₆ hydroxyalkyl. In some embodiments, D has the formula D1b-XII, wherein Y¹ is a thiophene, substituted with hydroxyethyl. In some embodiments, D has the formula D1b-XII, wherein Y¹ is a thiophene, substituted with hydroxyethyl; and R^{b2} is methyl and R^{b3} is fluoro. In some embodiments, D has the formula D1b-XII, wherein Y¹ is a furan. In some embodiments, D has the formula D1b-XII, wherein Y¹ is an unsubstituted furan. In some embodiments, D has the formula D1b-XII, wherein Y¹ is a pyrrole. In some embodiments, D has the formula D1b-XII, wherein Y¹ is a substituted pyrrole. In some embodiments, D has the formula D1b-XII, wherein Y¹ is a pyrrole substituted by —S(O)₂—C₁-C₆ alkyl. In some embodiments, D has the formula D1b-XII, wherein Y¹ is a pyrrole substituted by —S(O)₂—CH₃. In some embodiments, D has the formula D1b-XII, wherein Y¹ is a pyridine. In some embodiments, D has the formula D1b-XII, wherein Y¹ is an unsubstituted pyridine. In some embodiments, D has the formula D1b-XII, wherein Y¹ is an isoxazole. In some embodiments, D has the formula D1b-XII, wherein Y¹ is an unsubstituted isoxazole. In some embodiments, D has the formula D1b-XII, wherein Y¹ is an isoxazole substituted by one or more C₁-C₆ alkyl. In some embodiments, D has the formula D1b-XII, wherein Y¹ is an isoxazole substituted by one or more methyl. In some embodiments, D has the formula D1b-XII, wherein Y¹ is an isoxazole substituted by one methyl group. In some embodiments, D has the formula D1b-XII, wherein Y¹ is an isoxazole substituted by two methyl groups.

In some embodiments, D has a formula selected from the group consisting of

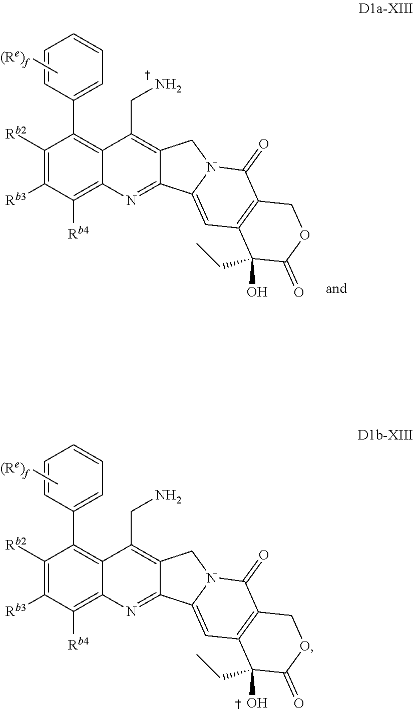

wherein
each R is independently selected from the group consisting of halogen, —OH, —NH₂, C₁-C₆ alkyl, C₁-C₆ hydroxyalkyl, —S(O)₂—C₁-C₆ alkyl, and —C(O)NH—C₁-C₆ alkyl;
f is 0, 1,2,3,4, or 5; and
the remaining variables are as defined for D₁ₐ and D_{1b}. In some embodiments, D has a structure corresponding to any of formulas D1a-XIII and D1b-XIII and variations thereof, wherein the NH₂ group is replaced by an OH group.

In some embodiments, D has the formula D1a-XIII, wherein R^{b2} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1a-XIII, wherein R^{b3} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1a-XIII, wherein R is selected from the group consisting of halogen, —OH, —NH₂, C₁-C₆ alkyl, C₁-C₆ hydroxyalkyl, —S(O)₂—C₁-C₆ alkyl, and —C(O)NH—C₁-C₆ alkyl. In some embodiments, D has the formula D1a-XIII, wherein f is 0, 1, 2, 3, 4, or 5. In some embodiments, D has the formula D1a-XIII, wherein f is 0. In some embodiments, D has the formula D1a-XIII, wherein f is 1. In some embodiments, D has the formula D1a-XIII, wherein f is 2. In some embodiments, D has the formula D1a-XIII, wherein f is 3. In some embodiments, D has the formula D1a-XIII, wherein f is 4. In some embodiments, D has the formula D1a-XIII, wherein f is 5.

In some embodiments, D has the formula D1a-XIII, wherein R^{b2} is C₁-C₆ alkyl. In some embodiments, D has the formula D1a-XIII, wherein R^{b2} is methyl. In some embodiments, D has the formula D1a-XIII, wherein R^{b3} is halo. In some embodiments, D has the formula D1a-XIII, wherein R^{b3} is fluoro. In some embodiments, D has the formula D1a-XIII, wherein R^{b2} is methyl and R^{b3} is fluoro. In some embodiments, D has the formula D1a-XIII, wherein R is —OH. In some embodiments, D has the formula D1a-XIII, wherein R is —OH and f is 1. In some embodiments, D has the formula D1a-XIII, wherein R is halo. In some embodiments, D has the formula D1a-XIII, wherein R is fluoro. In some embodiments, D has the formula D1a-XIII, wherein R is —NH₂. In some embodiments, D has the formula D1a-XIII, wherein R is —C(O)NH—C₁-C₆ alkyl.

In some embodiments, D has the formula D1b-XIII, wherein R^{b2} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1b-XIII, wherein R^{b3} is selected from the group consisting of H, halogen, C₁-C₆ alkyl, C₁-C₆ haloalkyl, —OR^{a}, —NHR^{a}, and —SR^{a}, wherein each R^{a} is independently selected from the group consisting of H, C₁-C₆ alkyl, and C₁-C₆ haloalkyl. In some embodiments, D has the formula D1b-XIII, wherein R is selected from the group consisting of halogen, —OH, —NH₂, C₁-C₆ alkyl, C₁-C₆ hydroxyalkyl, —S(O)₂—C₁-C₆ alkyl, and —C(O)NH—C₁-C₆ alkyl. In some embodiments, D has the formula D1b-XIII, wherein f is 0, 1, 2, 3, 4, or 5. In some embodiments, D has the formula D1b-XIII, wherein f is 0. In some embodiments, D has the formula D1b-XIII, wherein f is 1. In some embodiments, D has the formula D1b-XIII, wherein f is 2. In some embodiments, D has the formula D1b-XIII, wherein f is 3. In some embodiments, D has the formula D1b-XIII, wherein f is 4. In some embodiments, D has the formula D1b-XIII, wherein f is 5.

In some embodiments, D has the formula D1b-XIII, wherein R^{b2} is C₁-C₆ alkyl. In some embodiments, D has the formula D1b-XIII, wherein R^{b2} is methyl. In some embodiments, D has the formula D1b-XIII, wherein R^{b3} is halo. In some embodiments, D has the formula D1b-XIII, wherein R^{b3} is fluoro. In some embodiments, D has the formula D1b-XIII, wherein R^{b2} is methyl and R^{b3} is fluoro. In some embodiments, D has the formula D1b-XIII, wherein R is —OH. In some embodiments, D has the formula D1b-XIII, wherein R is —OH and f is 1. In some embodiments, D has the formula D1b-XIII, wherein R^{e} is halo. In some embodiments, D has the formula D1b-XIII, wherein R^{e} is fluoro. In some embodiments, D has the formula D1b-XIII, wherein R^{e} is —NH₂. In some embodiments, D has the formula D1b-XIII, wherein R is —C(O)NH—C₁-C₆ alkyl.

In some embodiments, D has a formula selected from the group consisting of

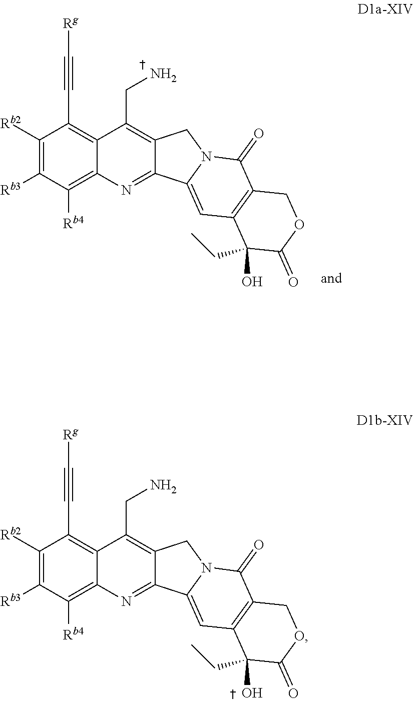

wherein
R^{g} is H, C₁-C₆ alkyl, or 3 to 8-membered heterocyclyl; and the remaining variables are as defined for D₁ₐ and D_{1b}. In some embodiments, R^{g} is C₁-C₆ alkyl. In some embodiments, D has a structure corresponding to any of formulas D1a-XIV and D1b-XIV and variations thereof, wherein the NH₂ group is replaced by an OH group.

In some embodiments, D has the formula D1a-XIV, wherein R^{b2} is H, halogen, C₁-C₆ alkyl, C₁-C₆ haloalkyl, —OR^{a}, —NHR^{a}, or —SRI; wherein each R^{a} is independently selected from the group consisting of H, C₁-C₆ alkyl, and C₁-C₆ haloalkyl. In some embodiments, D has the formula D1a-XIV, wherein R^{b3} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1a-XIV, wherein R^{g} is H, C₁-C₆ alkyl, or 3- to 8-membered heterocyclyl.

In some embodiments, D has the formula D1a-XIV, wherein R^{b2} is C₁-C₆ alkyl. In some embodiments, D has the formula D1a-XIV, wherein R^{b2} is methyl. In some embodiments, D has the formula D1a-XIV, wherein R^{b3} is halo. In some embodiments, D has the formula D1a-XIV, wherein R^{b3} is fluoro. In some embodiments, D has the formula D1a-XIV, wherein R^{b2} is methyl and R^{b3} is fluoro. In some embodiments, D has the formula D1a-XIV, wherein R^{g} is H. In some embodiments, D has the formula D1a-XIV, wherein R^{g} is C₁-C₆ alkyl. In some embodiments, D has the formula D1a-XIV, wherein R^{g} is 3- to 8-membered heterocyclyl. In some embodiments, D has the formula D1a-XIV, wherein R^{g} is H, R^{b2} is methyl, and R^{b3} is fluoro.

In some embodiments, D has the formula D1b -XIV, wherein R^{b2} is H, halogen, C₁-C₆ alkyl, C₁-C₆ haloalkyl, —OR^{a}, —NHR^{a}, or —SRI; wherein each R^{a} is independently selected from the group consisting of H, C₁-C₆ alkyl, and C₁-C₆ haloalkyl. In some embodiments, D has the formula D1b -XIV, wherein R^{b3} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1b-XIV, wherein R^{g} is H, C₁-C₆ alkyl, or 3- to 8-membered heterocyclyl.

In some embodiments, D has the formula D1b-XIV, wherein R^{b2} is C₁-C₆ alkyl. In some embodiments, D has the formula D1b-XIV, wherein R^{b2} is methyl. In some embodiments, D has the formula D1b-XIV, wherein R^{b3} is halo. In some embodiments, D has the formula D1b-XIV, wherein R^{b3} is fluoro. In some embodiments, D has the formula D1b-XIV, wherein R^{b2} is methyl and R^{b3} is fluoro. In some embodiments, D has the formula D1b-XIV, wherein R^{g} is H. In some embodiments, D has the formula D1b-XIV, wherein R^{g} is C₁-C₆ alkyl. In some embodiments, D has the formula D1b-XIV, wherein R^{g} is 3- to 8-membered heterocyclyl. In some embodiments, D has the formula D1b-XIV, wherein R^{g} is H, R^{b2} is methyl, and R^{b3} is fluoro.

In some embodiments, D has a formula selected from the group consisting of

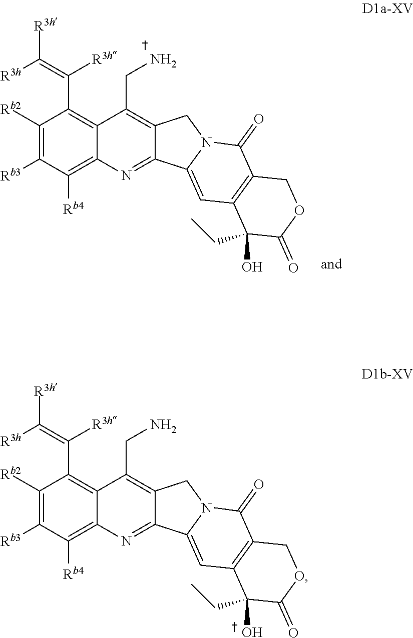

wherein
R^{3h}, R^{3h}′, and R^{3h}″ are each independently selected from the group consisting of H, C₁-C₆ alkyl, C₁-C₆ hydroxyalkyl, C₁-C₆ aminoalkyl, —C(O)—C₁-C₆ alkyl, —C(O)O—C₁-C₆ alkyl, —C(O)NH—C₁-C₆ alkyl, C₆-C₁₀ aryl, —C₆-C₁₀ aryl-C₁-C₆ alkyl, and —C₆-C₁₀ aryl-C₁-C₆ alkoxy; each optionally substituted with, C₁-C₆ alkyl, C₁-C₆ haloalkyl, —OR^{a}, —NR^{a}R^{a}, and —SR^{a}; and the remaining variables are as defined for D₁ₐ and D_{1b}. In some embodiments, D has a structure corresponding to any of formulas D1a-XV and D1b-XV and variations thereof, wherein the NH₂ group is replaced by an OH group.

In some embodiments, D has the formula D1a-XV, wherein R^{b2} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1a-XV, wherein R^{b3} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl.

In some embodiments, D has the formula D1a-XV, wherein R^{b2} is C₁-C₆ alkyl. In some embodiments, D has the formula D1a-XV, wherein R^{b2} is methyl. In some embodiments, D has the formula D1a-XV, wherein R^{b2} is —OH. In some embodiments, D has the formula D1a-XV, wherein R^{b2} is halo. In some embodiments, D has the formula D1a-XV, wherein R^{b2} is fluoro. In some embodiments, D has the formula D1a-XV, wherein R^{b3} is halo. In some embodiments, D has the formula D1a-XV, wherein R^{b3} is fluoro. In some embodiments, D has the formula D1a-XV, wherein R^{b2} is methyl and R^{b3} is fluoro. In some embodiments, D has the formula D1a-XV, wherein R^{b2} is H and R^{b3} is fluoro. In some embodiments, D has the formula D1a-XV, wherein R^{b2} and R^{b3} are both fluoro. In some embodiments, D has the formula D1a-XV, wherein R^{b2} is —OH and R^{b3} is H. In some embodiments, D has the formula D1a-XV, wherein R^{3h}, R^{3h}′, and R^{3h}″ are each H. In some embodiments, D has the formula D1a-XV, wherein R^{3h} and R^{3h}′ are both H and R^{3h}″ is C₁-C₆ alkyl. In some embodiments, D has the formula D1a-XV, wherein R^{3h} and R^{3h}′ are both H and R^{3h}″ is methyl. In some embodiments, D has the formula D1a-XV, wherein R^{3h} and R^{3h}′ are both C₁-C₆ alkyl and R^{3h}″ is H. In some embodiments, D has the formula D1a-XV, wherein R^{3h} and R^{3h}′ are both methyl and R^{3h}″ is H. In some embodiments, D has the formula D1a-XV, wherein R^{3h} is H, and R^{3h}′ and R^{3h}″ are both methyl. In some embodiments, D has the formula D1a-XV, wherein R^{b2} is methyl, R^{b3} is fluoro, and R^{3h}, R^{3h}′ and R^{3h}″ are each H. In some embodiments, D has the formula D1a-XV, wherein R^{b2} is methyl, R^{b3} is fluoro, R^{3h} and R^{3h}′ are both H, and R^{3h}″ is methyl. In some embodiments, D has the formula D1a-XV, wherein R^{3h} and R^{3h}″ are both H, and R^{3h}′ is —C₆-C₁₀ aryl-C₁-C₆ alkoxy.

In some embodiments, D has the formula D1b-XV, wherein R^{b2} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1b-XV, wherein R^{b3} is H, halogen, C₁-C₆ alkyl, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl.

In some embodiments, D has the formula D1b-XV, wherein R^{b2} is C₁-C₆ alkyl. In some embodiments, D has the formula D1b-XV, wherein R^{b2} is methyl. In some embodiments, D has the formula D1b-XV, wherein R^{b2} is —OH. In some embodiments, D has the formula D1b-XV, wherein R^{b2} is halo. In some embodiments, D has the formula D1b-XV, wherein R^{b2} is fluoro. In some embodiments, D has the formula D1b-XV, wherein R^{b3} is halo. In some embodiments, D has the formula D1b-XV, wherein R^{b3} is fluoro. In some embodiments, D has the formula D1b-XV, wherein R^{b2} is methyl and R^{b3} is fluoro. In some embodiments, D has the formula D1b-XV, wherein R^{b2} is H and R^{b3} is fluoro. In some embodiments, D has the formula D1b-XV, wherein R^{b2} and R^{b3} are both fluoro. In some embodiments, D has the formula D1b-XV, wherein R^{b2} is —OH and R^{b3} is H. In some embodiments, D has the formula D1b-XV, wherein R^{3h}, R^{3h}′ and R^{3h}″ are each H. In some embodiments, D has the formula D1b-XV, wherein R^{3h} and R^{3h}′ are both H and R^{3h}″ is C₁-C₆ alkyl. In some embodiments, D has the formula D1b-XV, wherein R^{3h} and R^{3h}′ are both H and R^{3h}″ is methyl. In some embodiments, D has the formula D1b-XV, wherein R^{3h} and R^{3h}′ are both C₁-C₆ alkyl and R^{3h}″ is H. In some embodiments, D has the formula D1b-XV, wherein R^{3h} and R^{3h}′ are both methyl and R^{3h}″ is H. In some embodiments, D has the formula D1b-XV, wherein R^{3h} is H, and R^{3h}′ and R^{3h}″ are both methyl. In some embodiments, D has the formula D1b-XV, wherein R^{b2} is methyl, R^{b3} is fluoro, and R^{3h}, R^{3h}′ and R^{3h}″ are each H. In some embodiments, D has the formula D1b-XV, wherein R^{b2} is methyl, R^{b3} is fluoro, R^{3h} and R^{3h}′ are both H, and R^{3h}″ is methyl. In some embodiments, D has the formula D1b-XV, wherein R^{3h} and R^{3h}″ are both H, and R^{3h}′ is —C₆-C₁₀ aryl-C₁-C₆ alkoxy.

In some embodiments, D has a formula selected from the group consisting of

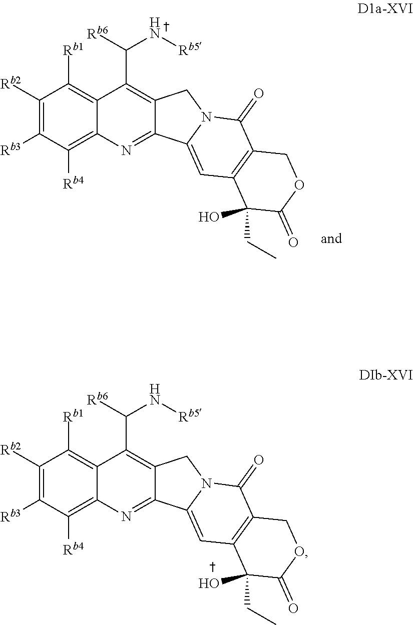

wherein the variables are as defined for D₁ₐ and D_{1b}. In some embodiments, D has structure corresponding to any of formulas D1a-XVI and D1b-XVI and variations thereof, wherein the nitrogen atom to which R^{b5}′ is bound is replaced by an oxygen atom and R^{b5}′ is absent.

In some embodiments, D has the formula D1a-XVI, wherein R^{b1} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, R^{b1} is H, halogen, —CN, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SRI; wherein each R^{a} is independently selected from the group consisting of H, C₁-C₆ alkyl, and C₁-C₆ haloalkyl. In some embodiments, D has the formula D1a-XVI, wherein R^{b2} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1a-XVI, wherein R^{b3} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1a-XVI, wherein R^{b2} and R^{b3} are taken together to form a methylenedioxy moiety. In some embodiments, D has the formula D1a-XVI, wherein R^{b6} is H or is taken together with R^{b1} to form a carbocyclo or heterocyclo. In some embodiments, D has the formula D1a-XVI, wherein R^{b5}′ is H, —C(O)—C₁-C₆ alkyl, or —C(O)—C₁-C₆ alkylamino.

In some embodiments, D has the formula D1a-XVI, wherein R^{b1} is halo. In some embodiments, D has the formula D1a-XVI, wherein R^{b1} is fluoro. In some embodiments, D has the formula D1a-XVI, wherein R^{b1} is bromo. In some embodiments, D has the formula D1a-XVI, wherein R^{b1} is chloro. In some embodiments, D has the formula D1a-XVI, wherein R^{b1} is —CN. In some embodiments, D has the formula D1a-XVI, wherein R^{b1} is C₁-C₆ alkyl. In some embodiments, D has the formula D1a-XVI, wherein R^{b1} is methyl.

In some embodiments, D has the formula D1a-XVI, wherein R^{b2} is C₁-C₆ alkyl. In some embodiments, D has the formula D1a-XVI, wherein R^{b2} is methyl. In some embodiments, D has the formula D1a-XVI, wherein R^{b2} is halo. In some embodiments, D has the formula D1a-XVI, wherein R^{b2} is chloro. In some embodiments, D has the formula D1a-XVI, wherein R^{b2} is bromo. In some embodiments, D has the formula D1a-XVI, wherein R^{b2} is fluoro. In some embodiments, D has the formula D1a-XVI, wherein R^{b2} is C₁-C₆ alkoxy. In some embodiments, D has the formula D1a-XVI, wherein R^{b2} is methoxy. In some embodiments, D has the formula D1a-XVI, wherein R^{b2} is C₁-C₆ hydroxyalkyl. In some embodiments, D has the formula D1a-XVI, wherein R^{b2} is C₁-C₆ haloalkyl. In some embodiments, D has the formula D1a-XVI, wherein R^{b2} is trihalomethyl. In some embodiments, D has the formula D1a-XVI, wherein R^{b2} is trifluoromethyl. In some embodiments, D has the formula D1a-XVI, wherein R^{b2} is C₂-C₆ alkenyl. In some embodiments, D has the formula D1a-XVI, wherein R^{b2} is —OH. In some embodiments, D has the formula D1a-XVI, wherein R^{b2} is —SR^{a}. In some embodiments, D has the formula D1a-XVI, wherein R^{b2} is —SR^{a}, wherein R^{a} is C₁-C₆ alkyl. In some embodiments, D has the formula D1a-XVI, wherein R^{b2} is —SR^{a}, wherein R^{a} is methyl. In some embodiments, D has the formula D1a-XVI, wherein R^{b2} is —SR^{a}, wherein R^{a} is C₁-C₆ haloalkyl. In some embodiments, D has the formula D1a-XVI, wherein R^{b2} is —SR^{a}, wherein R^{a} is trihalomethyl. In some embodiments, D has the formula D1a-XVI, wherein R^{b2} is —SR^{a}, wherein R^{a} is trifluoromethyl.

In some embodiments, D has the formula D1a-XVI, wherein R^{b3} is halo. In some embodiments, D has the formula D1a-XVI, wherein R^{b3} is chloro. In some embodiments, D has the formula D1a-XVI, wherein R^{b3} is bromo. In some embodiments, D has the formula D1a-XVI, wherein R^{b3} is fluoro. In some embodiments, D has the formula D1a-XVI, wherein R^{b3} is C₁-C₆ alkyl. In some embodiments, D has the formula D1a-XVI, wherein R^{b3} is methyl. In some embodiments, D has the formula D1a-XVI, wherein R^{b3} is ethyl. In some embodiments, D has the formula D1a-XVI, wherein R^{b3} is C₁-C₆ alkoxy. In some embodiments, D has the formula D1a-XVI, wherein R^{b3} is methoxy.

In some embodiments, D has the formula D1a-XVI, wherein R^{b2} and R^{b3} are taken together with their intervening atoms to form 5-membered heterocyclo fused with 6-membered aryl. In some embodiments, D has the formula D1a-XVI, wherein R^{b2} and R^{b3} are taken together with their intervening atoms to form 2,3-dihydrobenzofuranyl.

In some embodiments, D has the formula D1a-XVI, wherein R^{b1} and R^{b6} are taken together with their intervening atoms to form a carbocyclo. In some embodiments, D has the formula D1a-XVI, wherein R^{b1} and R^{b6} are taken together with their intervening atoms to form a 6-membered cycloalkyl.

In some embodiments, D has the formula D1a-XVI, wherein R^{b5}′ is H. In some embodiments, D has the formula D1a-XVI, wherein R^{b5}′ is H. In some embodiments, D has the formula D1a-XVI, wherein R^{b5}′ is —C(O)—C₁-C₆ alkyl. In some embodiments, D has the formula D1a-XVI, wherein R^{b5}′ is —C(O)—C₁-C₆ alkylamino. In some embodiments, D has the formula D1a-XVI, wherein R^{b5}′ is 3- to 10-membered heteroaryl substituted with C₁-C₆ hydroxyalkyl. In some embodiments, D has the formula D1a-XVI, wherein R^{b5}′ is 5- to 6-membered heteroaryl substituted with C₁-C₆ hydroxyalkyl. In some embodiments, D has the formula D1a-XVI, wherein R^{b5}′ is pyridinyl substituted with —CH₂OH.

In some embodiments, D has the formula D1b-XVI, wherein R^{b1} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, R^{b1} is H, halogen, —CN, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1b-XVI, wherein R^{b2} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1b-XVI, wherein R^{b3} is H, halogen, —OH, C₁-C₆ alkyl, C₂-C₆ alkenyl, C₂-C₆ alkynyl, C₁-C₆ alkoxy, C₁-C₆ haloalkyl, —OR^{a}, —NHR^{a}, or —SR^{a}; wherein each R^{a} is independently selected from the group consisting of H, C₁-C₆ alkyl, and C₁-C₆ haloalkyl. In some embodiments, D has the formula D1b-XVI, wherein R^{b2} and R^{b3} are taken together to form a methylenedioxy moiety. In some embodiments, D has the formula D1b-XVI, wherein R^{b6} is H or is taken together with R^{b1} to form a carbocyclo or heterocyclo. In some embodiments, D has the formula D1b-XVI, wherein R^{b5}′ is H, —C(O)—C₁-C₆ alkyl, or —C(O)—C₁-C₆ alkylamino.

In some embodiments, D has the formula D1b-XVI, wherein R^{b1} is halo. In some embodiments, D has the formula D1b-XVI, wherein R^{b1} is fluoro. In some embodiments, D has the formula D1b-XVI, wherein R^{b1} is bromo. In some embodiments, D has the formula D1b-XVI, wherein R^{b1} is chloro. In some embodiments, D has the formula D1b-XVI, wherein R^{b1} is —CN. In some embodiments, D has the formula D1b-XVI, wherein R^{b1} is C₁-C₆ alkyl. In some embodiments, D has the formula D1b-XVI, wherein R^{b1} is methyl.

In some embodiments, D has the formula D1b-XVI, wherein R^{b2} is C₁-C₆ alkyl. In some embodiments, D has the formula D1b-XVI, wherein R^{b2} is methyl. In some embodiments, D has the formula D1b-XVI, wherein R^{b2} is halo. In some embodiments, D has the formula D1b-XVI, wherein R^{b2} is chloro. In some embodiments, D has the formula D1b-XVI, wherein R^{b2} is bromo. In some embodiments, D has the formula D1b-XVI, wherein R^{b2} is fluoro. In some embodiments, D has the formula D1b-XVI, wherein R^{b2} is C₁-C₆ alkoxy.

In some embodiments, D has the formula D1b-XVI, wherein R^{b2} is methoxy. In some embodiments, D has the formula D1b-XVI, wherein R^{b2} is C₁-C₆ haloalkyl. In some embodiments, D has the formula D1b-XVI, wherein R^{b2} is C₁-C₆ hydroxyalkyl. In some embodiments, D has the formula D1b-XVI, wherein R^{b2} is trihalomethyl. In some embodiments, D has the formula D1b-XVI, wherein R^{b2} is trifluoromethyl. In some embodiments, D has the formula D1b-XVI, wherein R^{b2} is C₂-C₆ alkenyl. In some embodiments, D has the formula D1b-XVI, wherein R^{b2} is —OH. In some embodiments, D has the formula D1b-XVI, wherein R^{b2} is —SR^{a}. In some embodiments, D has the formula D1b-XVI, wherein R^{b2} is —SR^{a}, wherein R^{a} is C₁-C₆ alkyl. In some embodiments, D has the formula D1b-XVI, wherein R^{b2} is —SR^{a}, wherein R^{a} is methyl. In some embodiments, D has the formula D1b-XVI, wherein R^{b2} is —SR^{a}, wherein R^{a} is C₁-C₆ haloalkyl. In some embodiments, D has the formula D1b-XVI, wherein R^{b2} is —SR^{a}, wherein R^{a} is trihalomethyl. In some embodiments, D has the formula D1b-XVI, wherein R^{b2} is —SR^{a}, wherein R^{a} is trifluoromethyl.

In some embodiments, D has the formula D1b-XVI, wherein R^{b3} is halo. In some embodiments, D has the formula D1b-XVI, wherein R^{b3} is chloro. In some embodiments, D has the formula D1b-XVI, wherein R^{b3} is bromo. In some embodiments, D has the formula D1b-XVI, wherein R^{b3} is fluoro. In some embodiments, D has the formula D1b-XVI, wherein R^{b3} is C₁-C₆ alkyl. In some embodiments, D has the formula D1b-XVI, wherein R^{b3} is methyl. In some embodiments, D has the formula D1b-XVI, wherein R^{b3} is ethyl. In some embodiments, D has the formula D1b-XVI, wherein R^{b3} is C₁-C₆ alkoxy. In some embodiments, D has the formula D1b-XVI, wherein R^{b3} is methoxy.

In some embodiments, D has the formula D1b-XVI, wherein R^{b2} and R^{b3} are taken together with their intervening atoms to form a 5-membered heterocyclo fused with 6-membered heteroaryl. In some embodiments, D has the formula D1b-XVI, wherein R^{b2} and R^{b3} are taken together with their intervening atoms to form 2,3-dihydrobenzofuranyl.

In some embodiments, D has the formula D1b-XVI, wherein R^{b1} and R^{b6} are taken together with their intervening atoms to form a carbocyclo. In some embodiments, D has the formula D1b-XVI, wherein R^{b1} and R^{b6} are taken together with their intervening atoms to form a 6-membered cycloalkyl.

In some embodiments, D has the formula D1b-XVI, wherein R^{b5}′ is H. In some embodiments, D has the formula D1b-XVI, wherein R^{b5}′ is H. In some embodiments, D has the formula D1b-XVI, wherein R^{b5}′ is —C(O)—C₁-C₆ alkyl. In some embodiments, D has the formula D1b-XVI, wherein R^{b5}′ is —C(O)—C₁-C₆ alkylamino. In some embodiments, D has the formula D1b-XVI, wherein R^{b5}′ is 3- to 10-membered heteroaryl substituted with C₁-C₆ hydroxyalkyl. In some embodiments, D has the formula D1b-XVI, wherein R^{b5}′ is 5- to 6-membered heteroaryl substituted with C₁-C₆ hydroxyalkyl. In some embodiments, D has the formula D1b-XVI, wherein R^{b5}′ is pyridinyl substituted with —CH₂OH.

In another embodiment, a Camptothecin Conjugate is provided having a formula:

L-(Q-D)p

or a salt thereof, wherein
L is a Ligand Unit;
subscript p is an integer of from 1 to 16;
Q is a Linker Unit having a formula selected from the group consisting of:
   Z-A-, -Z-A-RL-, -Z-A-RL-Y-, -Z-A-S*-RL-, -Z-A-S*-RL-Y-, Z-A-S*-W-, -Z-A-S*-W-RL-, -Z-A-B(S*)-RL-, -Z-A-B(S*)-W-, -Z-A-B(S*)-W-RL- and -Z-A-B(S*)-RL-Y-,
wherein Z is a Stretcher Unit;
A is a bond or a Connector Unit;
B is a Parallel Connector Unit;
S* is a Partitioning Agent;
RL is a Releasable Linker;
W is an Amino Acid Unit;
Y is a Spacer Unit; and
D is a is a Drug Unit having a formula of

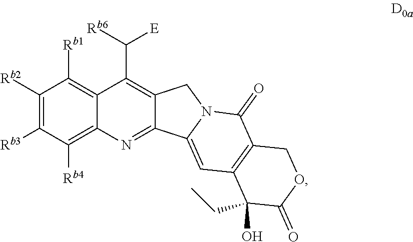

or a salt thereof; wherein;
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo;, or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a}; or
R^{b3} is combined with R^{b2} and the intervening atoms to form a 5- or 6-membered carbocyclo or heterocyclo or a 5- or 6-membered heterocyclo fused with 6-membered aryl;
R^{b4} is selected from the group consisting of H and halogen;
each R^{b5} and R^{b5}′ are independently selected from the group consisting of H, C₁-C₈ alkyl, C₁-C₈ hydroxyalkyl, C₁-C₆ alkyl-O—C₁-C₆alkyl-, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ hydroxyalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, heteroaryl-C₁-C₆ hydroxyalkyl, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane, R^{b5} is H and R^{b5} is C₁-C₆ alkyl-O—C₁-C₆ alkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆alkoxy-C(O)—C₁-C₈ aminoalkyl-, and C₁-C₈ aminoalkyl;
b) when E is NH₂, R^{b1} is selected from the group consisting of —CN, 5- to 12-membered heteroaryl with at least one annular N, 3- to 10-membered heterocycloalkyl with at least one annular N, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}, or R^{b1} is combined with R^{b2} and the intervening atoms to form a 5-, 6-, or 7-membered heterocyclo with at least one annular N, or 5- or 6-membered heterocyclo fused with 6-membered aryl; R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a} —NR^{a}R^{a}′, and —SR^{a}; or
R^{b2} is combined with R^{b1} or R^{b3} and the intervening atoms to form a 5- or 6-membered heterocyclo with at least one annular N, or R^{b2} is combined with R^{b1} or R^{b3} and the intervening atoms to form 5- or 6-membered heterocyclo fused with 6-membered aryl;
c) when R^{b2} is —OH or methyl and R^{b3} is F, then R^{b1} does not come together with R^{b5} or R^{b5}′ and the intervening atoms to form a ring; and
d) D is not selected from the group n consisting of (S)-7-ethyl-7-hydroxy-14-((4-methylpiperazin-1-yl)methyl)-10,13-dihydro-11 H-[1,3]dioxolo[4,5-g]pyrano[3′,4′:6,7]indolizino[1,2-b]quinoline-8,11(7H)-dione, (S)-7-ethyl-7-hydroxy-14-(morpholinomethyl)-10,13-dihydro-11H-[1,3]dioxolo[4,5-g]pyrano[3′,4′:6,7]indolizino[1,2-b]quinoline-8,11(7H)-dione, (S)-14-((4-(2-aminoethyl)piperazin-1-yl)methyl)-7-ethyl-7-hydroxy-10,13-dihydro-11 H-[1,3]dioxolo[4,5-g]pyrano[3′,4′:6,7]indolizino[1,2-b]quinoline-8,11(7H)-dione, (S)-14-((4-aminopiperidin-1-yl)methyl)-7-ethyl-7-hydroxy-10,13-dihydro-11 H-[1,3]dioxolo[4,5-g]pyrano[3′,4′:6,7]indolizino[1,2-b]quinoline-8,11(7H)-dione, and tert-butyl (S)-(1-((7-ethyl-7-hydroxy-8,11-dioxo-7,8,11,13-tetrahydro-10H-[1,3]dioxolo[4,5-g]pyrano[3′,4′:6,7]indolizino[1,2-b]quinolin-14-yl)methyl)piperidin-4-yl)carbamate
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

In some embodiments, D has the formula of D₀-I

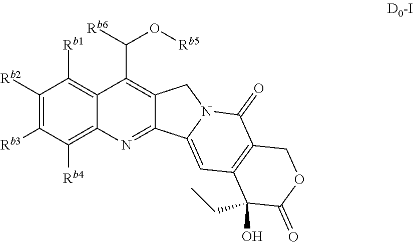

or a salt thereof; wherein R^{b1}—R^{b6} are each defined as for D₀.

In some embodiments, D has the formula of D₀-II

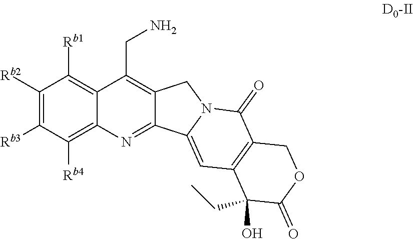

or a salt thereof; wherein;
R^{b1} is selected from the group consisting of —CN, 5- to 12-membered heteroaryl with at least one annular N, 3- to 10-membered heterocycloalkyl with at least one annular N, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2} and the intervening atoms to form a 5-, 6-, or 7-membered heterocyclo with at least one annular N, or 5- or 6-membered heterocyclo fused with 6-membered aryl;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered heterocyclo with at least one annular N, or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

In some embodiments, D has the formula of D₀-III

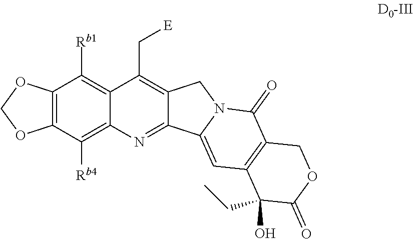

or a salt thereof; wherein R^{b1}, R^{b4}, and R^{b6} are each defined as for Doe.

In some embodiments, D has the formula of D₀-IV

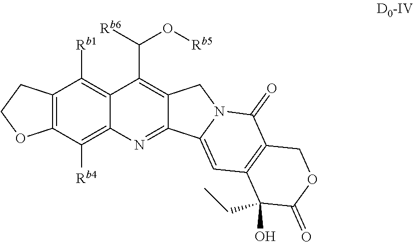

or a salt thereof; wherein R^{b1}, R^{b4}, R^{b5}, R^{b6} are each defined as for D₀ₐ. In some embodiments, R^{b1}, R^{b4}, R^{b5}, and R^{b6} are each H.

In some embodiments, D has the formula of D₀-V

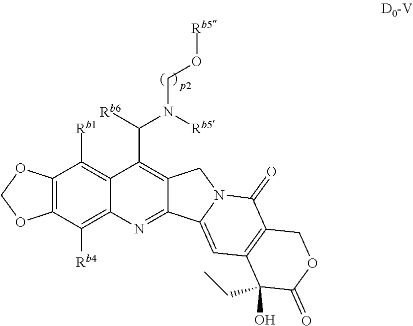

or a salt thereof; wherein:
p2 is 2 to 6;
R^{b5}″ is selected from the group consisting of H, C₁-C₆ alkyl and C₁-C₆ haloalkyl; and
R^{b1}, R^{b4}, R^{b5}′, R^{b6} are each defined as for Do. In some embodiments, p2 is 2, R^{b5}″ is methyl, and R^{b1}, R^{b4}, R^{b5}′, R^{b6} are each H.

In some embodiments, D has the formula of D₀-VI

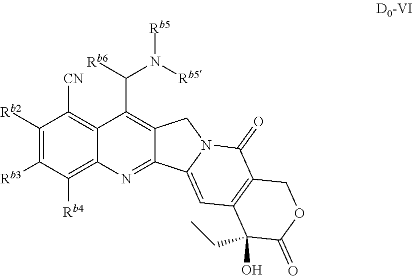

or a salt thereof; wherein R^{b2}, R^{b3}, R^{b4}, R^{b5}, R^{b5}′, and R^{b6} are each defined as for D₁ₐ. In some embodiments, R^{b2} is methyl, R^{b3} is F, and R^{b5}, R^{b5}′, and R^{b6} are each H.

In some embodiments, D has the formula of D₀-Vu

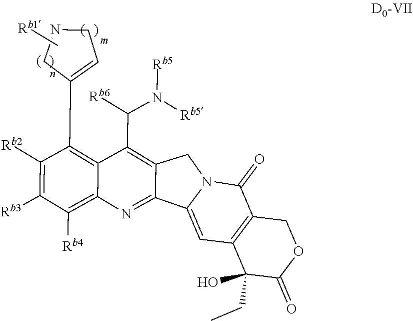

or a salt thereof; wherein;
m and n are each 1 or 2, and when m and n are both present, m+n is 2 or 3;
R^{b1}′ is C₁-C₃ alkyl or —C(O)R⁸; and
R^{b2}, R^{b3}, R^{b4}, R^{b5}, R^{b5}′, and R^{b6} are each defined as for D₀ₐ. In some embodiments, m is 1, n is 2, R^{b1} is methyl, and R^{b2}, R^{b3}, R^{b4}, R^{b5}, R^{b5}′, R^{b6} are each H. In some embodiments, m is 1, n is 2, R^{b1} is C(O)CH₃, and R^{b2}, R^{b3}, R^{b4}, R^{b5}, R^{b5}′, and R^{b6} are each H.

In some embodiments, D has the formula of D₀-VIII

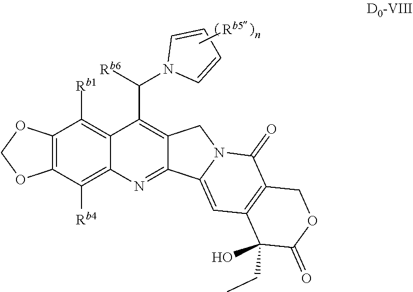

or a salt thereof; wherein:
n is 1,2 or 3;
R^{b5}″ is —C₁-C₆ hydroxyalkyl; and
R^{b1}, R^{M}, and R^{b6} are each defined as for D₀ₐ.
   In some embodiments, n is 1, R^{b5}″ is —CH₂OH, and R^{b1}, R^{b4}, and R^{b6} are each H.

In some embodiments, D has the formula of D₀-IX

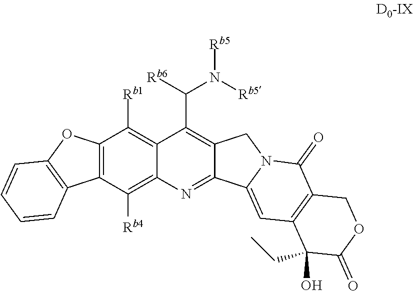

or a salt thereof; wherein R^{b1}, R^{b4}, R^{b5}, R^{b5}′, and R^{b6} are each defined as for D₁ₐ. In some embodiments, R^{b1}, R^{b4}, R^{b5}, R^{b5}′, and R^{b6} are each H.

In some embodiments, D has the formula of D₀-X

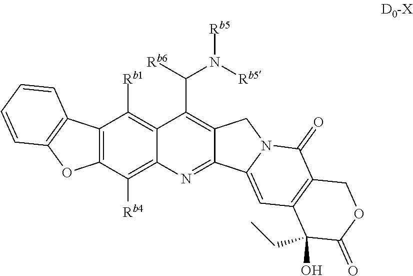

or a salt thereof; wherein R^{b1}, R^{b4}, R^{b5}, R^{b5}′, and R^{b6} are each defined as for D₀ₐ. In some embodiments, R^{b1}, R^{b4}, R^{b5}, R^{b5}′, and R^{b6} are each H.

In some embodiments, Do has a formula of Deb

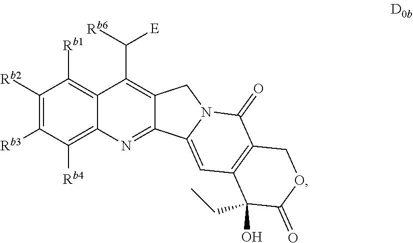

or a salt thereof; wherein;
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₈ alkyl-O—C₁-C₈ alkyl-, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, C₁-C₆ hydroxyalkyl-heteroaryl-, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane and E is —NR^{b5}R^{b5}′, then each R^{b5} and R^{b5}′ are independently selected from the group consisting of H, C₁-C₈ alkyl-O—C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
   b) when R^{b2} is combined with R^{b3} and the intervening atoms to form a 1,3-dioxolane, E is not —OH;
   c) when R^{b2} is methyl and R^{b3} is F, then R^{b1} does not come together with R^{b6} and the intervening atoms to form a ring, and
   d) D is not selected from the group consisting of (S)-7-ethyl-7-hydroxy-14-((4-methylpiperazin-1-yl)methyl)-10,13-dihydro-11 H-[1,3]dioxolo[4,5-g]pyrano[3′,4′:6,7]indolizino[1,2-b]quinoline-8,11(7H)-dione, and (S)-7-ethyl-7-hydroxy-14-(morpholinomethyl)-10,13-dihydro-11 H-[1,3]dioxolo[4,5-g]pyrano[3′,4′:6,7]indolizino[1,2-b]quinoline-8,11(7H)-dione.

In some embodiments, E is —NR^{b5}R^{b5}′. In some embodiments, E is —OR^{b5}.

In some embodiments, when R^{b2} is combined with R^{b3} and the intervening atoms to form a 1,3-dioxolane and E is —NR^{b5}R^{b5}′, then each R^{b5} and R^{b5}′ are independently selected from the group consisting of H, C₁-C₈ alkyl-O—C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heteroaryl having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₄ alkoxy-C(O)—NH—, C₁-C₆ alkoxy-C(O)—C₁-C₈ aminoalkyl-, and C₁-C₈ aminoalkyl.

In some embodiments, D has the formula of D_{0b}-I

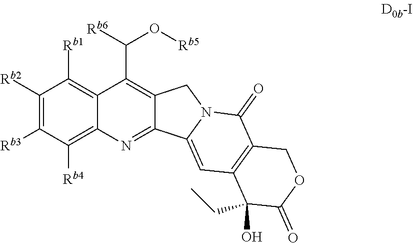

or a salt thereof; R^{b1}—R^{b6} are each defined as for Dab; and wherein when R^{b2} is combined with R^{b3} and the intervening atoms to form a 5-, 6-, or 7-membered heterocyclo, the heterocyclo has no more than one O.

In some embodiments, D has the formula of Dab-II

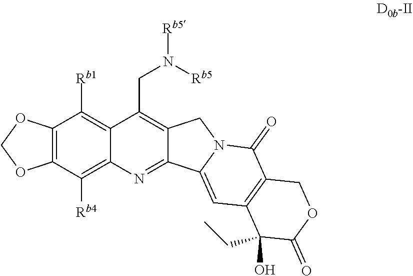

or a salt thereof; wherein;
R^{b5} is H and R^{b5}′ is selected from the group consisting of H, C₁-C₈ alkyl-O—C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂; and
R^{b1} and R^{b4} are each defined as for Dab, provided that

In some embodiments, D has the formula of D_{0b}-III

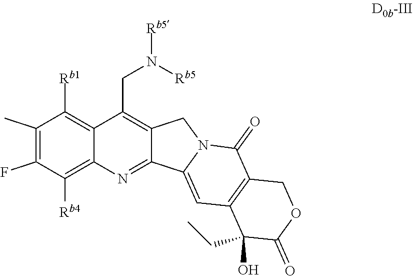

or a salt thereof; wherein R^{b1}, R^{b4}, R^{b5}, and R^{b5}′ are each defined as for D_{0b}.

In some embodiments, D has the formula of D_{0b}-I

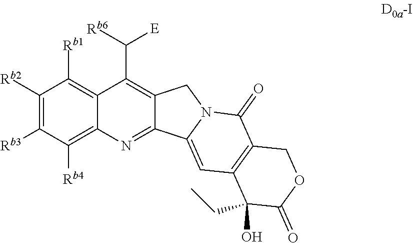

or a salt thereof; wherein;
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR′, —NR^{a}R^{a}′, and —SR^{a}; each optionally substituted with C₁-C₃ alkyl, —OR′, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a}; or
R^{b3} is combined with R^{b2} and the intervening atoms to form a 5- or 6-membered carbocyclo or heterocyclo or a 5- or 6-membered heterocyclo fused with 6-membered aryl;
R^{b4} is selected from the group consisting of H and halogen;
each R^{b5} and R^{b5}′ are independently selected from the group consisting of H, C₁-C₈ alkyl, C₁-C₈ hydroxyalkyl, C₁-C₈ alkyl-O—C₁-C₈ alkyl, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ hydroxyalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, heteroaryl-C₁-C₆ hydroxyalkyl, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a} are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane, and E is —NR^{b5}R^{b5}′, then each R^{b5} and R^{b5}′ are independently selected from the group consisting of H, C₁-C₈ alkyl-O—C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, phenyl-C(O)—, and phenyl-SO₂—, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
b) when R^{b2} is combined with R^{b3} and the intervening atoms to form a 1,3-dioxolane, E is not —OH;
c) when E is NH₂, R^{b1} is selected from the group consisting of —CN, 5- to 12-membered heteroaryl with at least one annular N, 3- to 10-membered heterocycloalkyl with at least one annular N, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —NR^{a}R^{a}′, and —SR^{a}; each optionally substituted with C₁-C₃ alkyl, —OR^{a}, —NR^{a}R^{a}, —C(O)R^{a}, and —SR^{a}, or R^{b1} is combined with R^{b2} and the intervening atoms to form a 5-, 6-, or 7-membered heterocyclo with at least one annular N, or 5- or 6-membered heterocyclo fused with 6-membered aryl;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered heterocyclo with at least one annular N, or R^{b2} is combined with R^{b1} or R^{b3} and the intervening atoms to form 5- or 6-membered heterocyclo fused with 6-membered aryl; and
d) when R^{b2} is —OH or methyl and R^{b3} is F, then R^{b1} does not come together with R^{b5}, R^{b5}′ or R^{b6} and the intervening atoms to form a ring; and
e) D is not selected from the group consisting of (S)-7-ethyl-7-hydroxy-14-((4-methylpiperazin-1-yl)methyl)-10,13-dihydro-11 H-[1,3]dioxolo[4,5-g]pyrano[3′,4′:6,7]indolizino[1,2-b]quinoline-8,11(7H)-dione, (S)-7-ethyl-7-hydroxy-14-(morpholinomethyl)-10,13-dihydro-11 H-[1,3]dioxolo[4,5-g]pyrano[3′,4′:6,7]indolizino[1,2-b]quinoline-8,11(7H)-dione, (S)-14-((4-(2-aminoethyl)piperazin-1-yl)methyl)-7-ethyl-7-hydroxy-10,13-dihydro-11 H-[1,3]dioxolo[4,5-g]pyrano[3′,4′:6,7]indolizino[1,2-b]quinoline-8,11(7H)-dione, (S)-14-((4-aminopiperidin-1-yl)methyl)-7-ethyl-7-hydroxy-10,13-dihydro-11H-[1,3]dioxolo[4,5-g]pyrano[3′,4′:6,7]indolizino[1,2-b]quinoline-8,11(7H)-dione, and tert-butyl (S)-(1-((7-ethyl-7-hydroxy-8,11-dioxo-7,8,11,13-tetrahydro-10H-[1,3]dioxolo[4,5-g]pyrano[3′,4′:6,7]indolizino[1,2-b]quinolin-14-yl)methyl)piperidin-4-yl)carbamate.
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

In some embodiments, E is —NR^{b5}R^{b5}′. In some embodiments, E is —OR^{b5}.

In some embodiments, D has the formula of D₀-I′

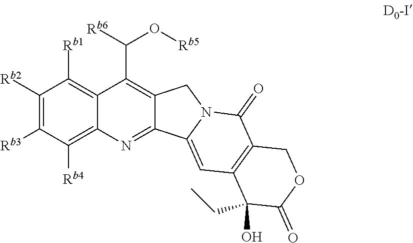

or a salt thereof; R^{b1}—R^{b6} are each defined as for Da-I; and wherein when R^{b2} is combined with R^{b3} and the intervening atoms to form a 5-, 6-, or 7-membered heterocyclo, the heterocyclo has no more than one O.

In some embodiments, D has the formula of D₀-HI′

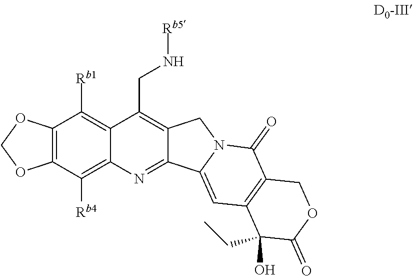

or a salt thereof; wherein each R^{b1}— R^{b4}, R^{b5}′ and R^{b6} are each defined as D₀ₐ-I.

In some embodiments, D is a compound of Table I or a salt thereof selected from the group consisting of:

TABLE I

| Compound | |
|---|---|
| No. | STRUCTURE |
| 2a | 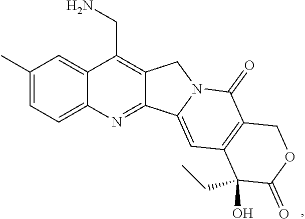 [SMILES: CC[C@@]1(O)C(=O)OCc2c1cc1n(c2=O)Cc2c-1nc1ccc(C)cc1c2CN] |
| 2b | 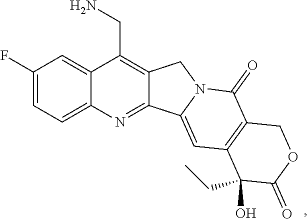 [SMILES: CC[C@@]1(O)C(=O)OCc2c1cc1n(c2=O)Cc2c-1nc1ccc(F)cc1c2CN] |
| 2c | 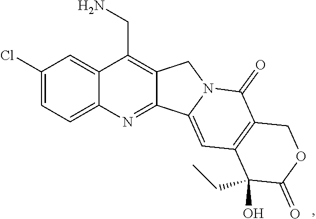 [SMILES: CC[C@@]1(O)C(=O)OCc2c1cc1n(c2=O)Cc2c-1nc1ccc(Cl)cc1c2CN] |
| 2d | 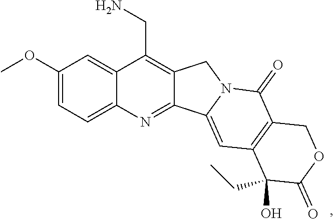 [SMILES: CC[C@@]1(O)C(=O)OCc2c1cc1n(c2=O)Cc2c-1nc1ccc(OC)cc1c2CN] |
| 2e | 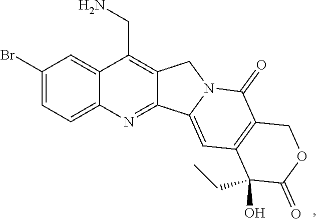 [SMILES: CC[C@@]1(O)C(=O)OCc2c1cc1n(c2=O)Cc2c-1nc1ccc(Br)cc1c2CN] |
| 2f | 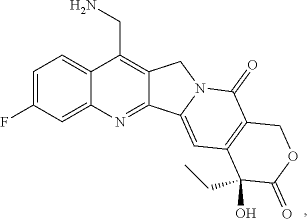 [SMILES: CC[C@@]1(O)C(=O)OCc2c1cc1n(c2=O)Cc2c-1nc1cc(F)ccc1c2CN] |
| 2g | 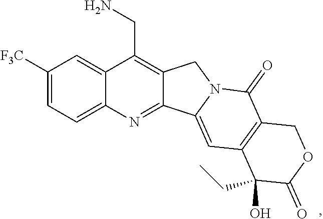 [SMILES: CC[C@@]1(O)C(=O)OCc2c1cc1n(c2=O)Cc2c-1nc1ccc(C(F)(F)F)cc1c2CN] |
| 2h | 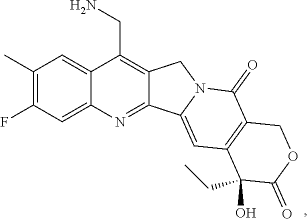 [SMILES: CC[C@@]1(O)C(=O)OCc2c1cc1n(c2=O)Cc2c-1nc1cc(F)c(C)cc1c2CN] |
| 2i | 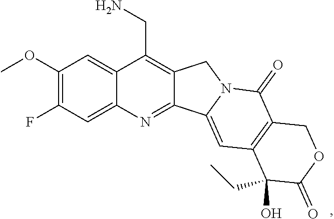 [SMILES: CC[C@@]1(O)C(=O)OCc2c1cc1n(c2=O)Cc2c-1nc1cc(F)c(OC)cc1c2CN] |
| 2j | 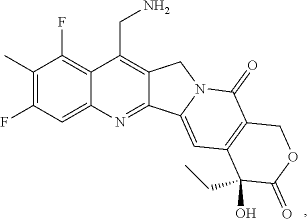 [SMILES: CC[C@@]1(O)C(=O)OCc2c1cc1n(c2=O)Cc2c-1nc1cc(F)c(C)c(F)c1c2CN] |
| 2k | 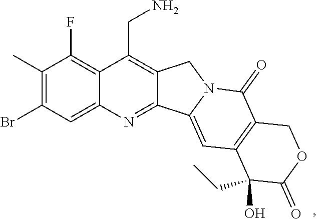 [SMILES: CC[C@@]1(O)C(=O)OCc2c1cc1n(c2=O)Cc2c-1nc1cc(Br)c(C)c(F)c1c2CN] |
| 2l | 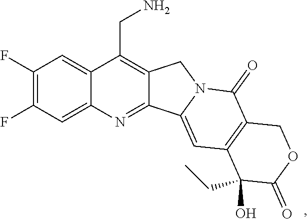 [SMILES: CC[C@@]1(O)C(=O)OCc2c1cc1n(c2=O)Cc2c-1nc1cc(F)c(F)cc1c2CN] |
| 2m | 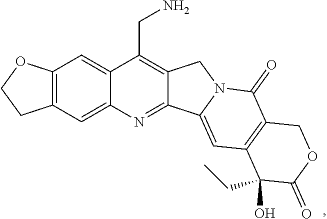 [SMILES: CC[C@@]1(O)C(=O)OCc2c1cc1n(c2=O)Cc2c-1nc1cc3c(cc1c2CN)OCC3] |
| 2n | 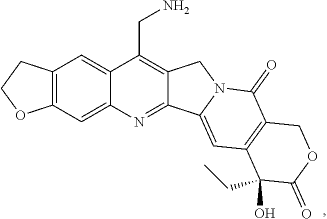 [SMILES: CC[C@@]1(O)C(=O)OCc2c1cc1n(c2=O)Cc2c-1nc1cc3c(cc1c2CN)CCO3] |
| 2o | 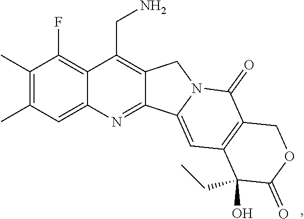 [SMILES: CC[C@@]1(O)C(=O)OCc2c1cc1n(c2=O)Cc2c-1nc1cc(C)c(C)c(F)c1c2CN] |
| 2p | 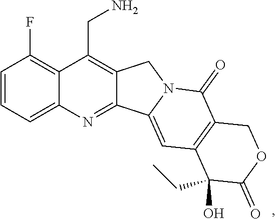 [SMILES: CC[C@@]1(O)C(=O)OCc2c1cc1n(c2=O)Cc2c-1nc1cccc(F)c1c2CN] |
| 2q | 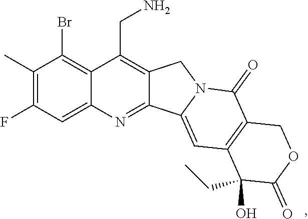 [SMILES: CC[C@@]1(O)C(=O)OCc2c1cc1n(c2=O)Cc2c-1nc1cc(F)c(C)c(Br)c1c2CN] |
| 2r | 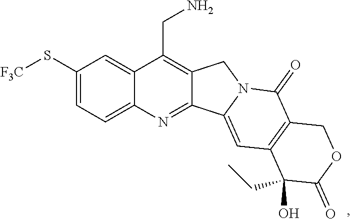 [SMILES: CC[C@@]1(O)C(=O)OCc2c1cc1n(c2=O)Cc2c-1nc1ccc(SC(F)(F)F)cc1c2CN] |
| 2s | 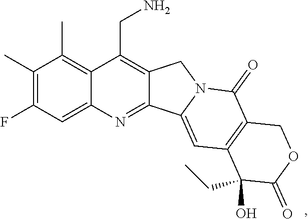 [SMILES: CC[C@@]1(O)C(=O)OCc2c1cc1n(c2=O)Cc2c-1nc1cc(F)c(C)c(C)c1c2CN] |
| 2t | 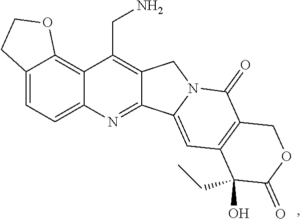 [SMILES: CC[C@@]1(O)C(=O)OCc2c1cc1n(c2=O)Cc2c-1nc1ccc3c(c1c2CN)OCC3] |
| 2u | 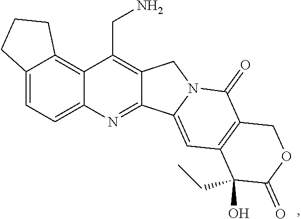 [SMILES: CC[C@@]1(O)C(=O)OCc2c1cc1n(c2=O)Cc2c-1nc1ccc3c(c1c2CN)CCC3] |
| 2v | 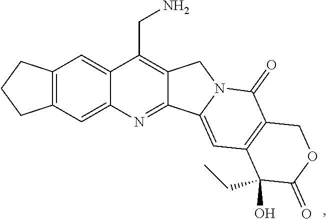 [SMILES: CC[C@@]1(O)C(=O)OCc2c1cc1n(c2=O)Cc2c-1nc1cc3c(cc1c2CN)CCC3] |
| 2w | 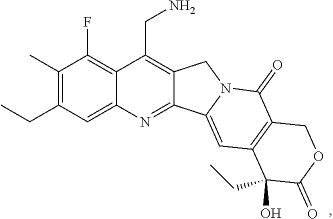 [SMILES: CCc1cc2nc3c(c(CN)c2c(F)c1C)Cn1c-3cc2c(c1=O)COC(=O)[C@]2(O)CC] |
| 2x | 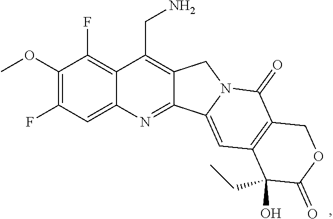 [SMILES: CC[C@@]1(O)C(=O)OCc2c1cc1n(c2=O)Cc2c-1nc1cc(F)c(OC)c(F)c1c2CN] |
| 2y | 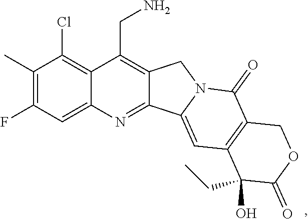 [SMILES: CC[C@@]1(O)C(=O)OCc2c1cc1n(c2=O)Cc2c-1nc1cc(F)c(C)c(Cl)c1c2CN] |
| 2z | 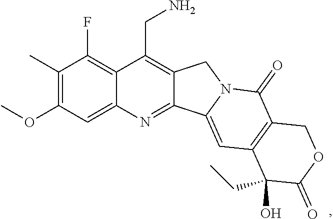 [SMILES: CC[C@@]1(O)C(=O)OCc2c1cc1n(c2=O)Cc2c-1nc1cc(OC)c(C)c(F)c1c2CN] |
| 2aa | 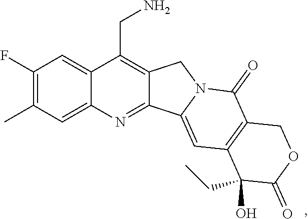 [SMILES: CC[C@@]1(O)C(=O)OCc2c1cc1n(c2=O)Cc2c-1nc1cc(C)c(F)cc1c2CN] |
| 2ab | 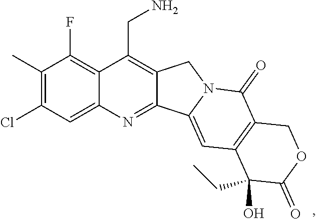 [SMILES: CC[C@@]1(O)C(=O)OCc2c1cc1n(c2=O)Cc2c-1nc1cc(Cl)c(C)c(F)c1c2CN] |
| 2ac | 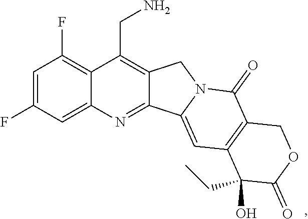 [SMILES: CC[C@@]1(O)C(=O)OCc2c1cc1n(c2=O)Cc2c-1nc1cc(F)cc(F)c1c2CN] |
| 2ad | 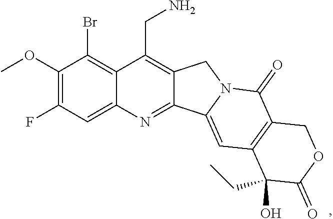 [SMILES: CC[C@@]1(O)C(=O)OCc2c1cc1n(c2=O)Cc2c-1nc1cc(F)c(OC)c(Br)c1c2CN] |
| 2ae | 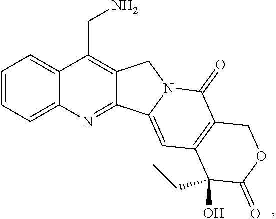 [SMILES: CC[C@@]1(O)C(=O)OCc2c1cc1n(c2=O)Cc2c-1nc1ccccc1c2CN] |
| 2af | 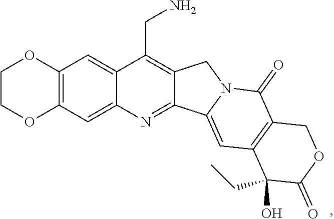 [SMILES: CC[C@@]1(O)C(=O)OCc2c1cc1n(c2=O)Cc2c-1nc1cc3c(cc1c2CN)OCCO3] |
| 2ag | 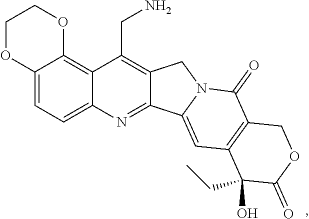 [SMILES: CC[C@@]1(O)C(=O)OCc2c1cc1n(c2=O)Cc2c-1nc1ccc3c(c1c2CN)OCCO3] |
| 2ah | 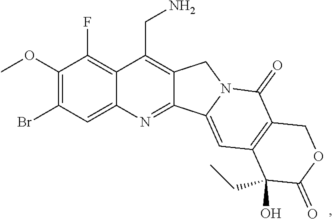 [SMILES: CC[C@@]1(O)C(=O)OCc2c1cc1n(c2=O)Cc2c-1nc1cc(Br)c(OC)c(F)c1c2CN] |
| 2ai | 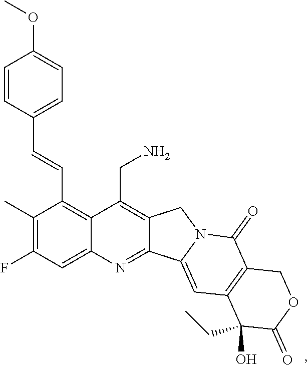 [SMILES: CC[C@@]1(O)C(=O)OCc2c1cc1n(c2=O)Cc2c-1nc1cc(F)c(C)c(/C=C/c3ccc(OC)cc3)c1c2CN] |
| 2aj | 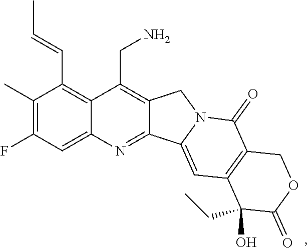 [SMILES: C/C=C/c1c(C)c(F)cc2nc3c(c(CN)c12)Cn1c-3cc2c(c1=O)COC(=O)[C@]2(O)CC] |
| 2ak | 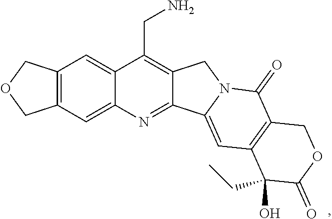 [SMILES: CC[C@@]1(O)C(=O)OCc2c1cc1n(c2=O)Cc2c-1nc1cc3c(cc1c2CN)COC3] |
| 2al | 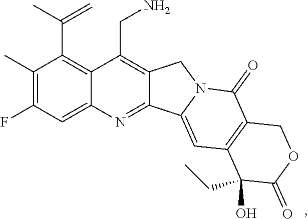 [SMILES: C=C(C)c1c(C)c(F)cc2nc3c(c(CN)c12)Cn1c-3cc2c(c1=O)COC(=O)[C@]2(O)CC] |
| 2am | 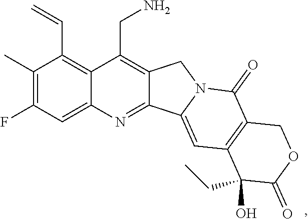 [SMILES: C=Cc1c(C)c(F)cc2nc3c(c(CN)c12)Cn1c-3cc2c(c1=O)COC(=O)[C@]2(O)CC] |
| 2an | 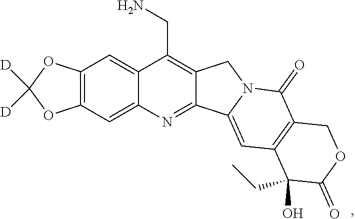 [SMILES: [2H]C1([2H])Oc2cc3nc4c(c(CN)c3cc2O1)Cn1c-4cc2c(c1=O)COC(=O)[C@]2(O)CC] |
| 2ao | 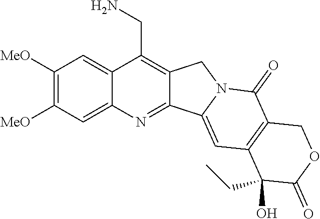 [SMILES: CC[C@@]1(O)C(=O)OCc2c1cc1n(c2=O)Cc2c-1nc1cc(OC)c(OC)cc1c2CN] |
| 2ap | 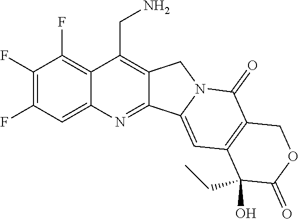 [SMILES: CC[C@@]1(O)C(=O)OCc2c1cc1n(c2=O)Cc2c-1nc1cc(F)c(F)c(F)c1c2CN] |
| 2aq | 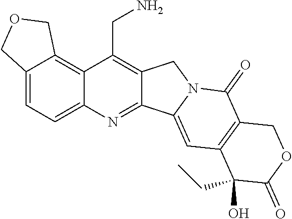 [SMILES: CC[C@@]1(O)C(=O)OCc2c1cc1n(c2=O)Cc2c-1nc1ccc3c(c1c2CN)COC3] |
| 2ar | 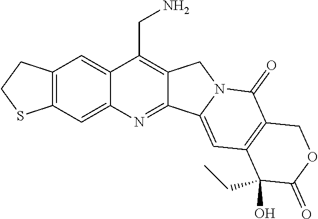 [SMILES: CC[C@@]1(O)C(=O)OCc2c1cc1n(c2=O)Cc2c-1nc1cc3c(cc1c2CN)CCS3] |
| 2as | 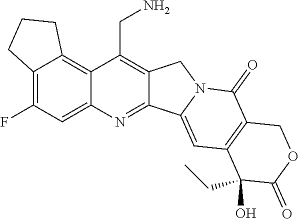 [SMILES: CC[C@@]1(O)C(=O)OCc2c1cc1n(c2=O)Cc2c-1nc1cc(F)c3c(c1c2CN)CCC3] |
| 2at | 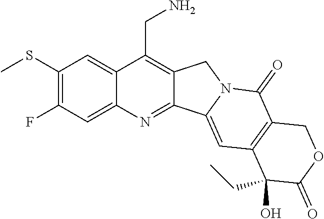 [SMILES: CC[C@@]1(O)C(=O)OCc2c1cc1n(c2=O)Cc2c-1nc1cc(F)c(SC)cc1c2CN] |
| 2au | 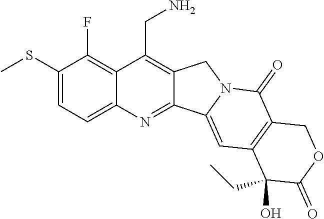 [SMILES: CC[C@@]1(O)C(=O)OCc2c1cc1n(c2=O)Cc2c-1nc1ccc(SC)c(F)c1c2CN] |
| 2av | 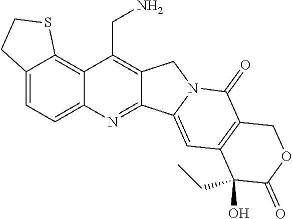 [SMILES: CC[C@@]1(O)C(=O)OCc2c1cc1n(c2=O)Cc2c-1nc1ccc3c(c1c2CN)SCC3] |
| 2aw | 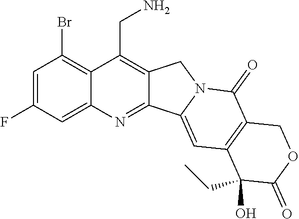 [SMILES: CC[C@@]1(O)C(=O)OCc2c1cc1n(c2=O)Cc2c-1nc1cc(F)cc(Br)c1c2CN] |
| 2ax | 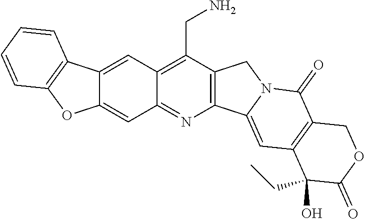 [SMILES: CC[C@@]1(O)C(=O)OCc2c1cc1n(c2=O)Cc2c-1nc1cc3oc4ccccc4c3cc1c2CN] |
| 2ay | 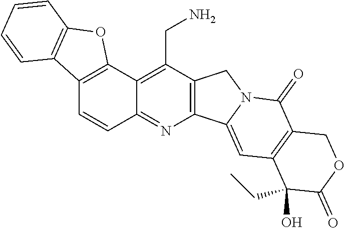 [SMILES: CC[C@@]1(O)C(=O)OCc2c1cc1n(c2=O)Cc2c-1nc1ccc3c4ccccc4oc3c1c2CN] |
| 2az | 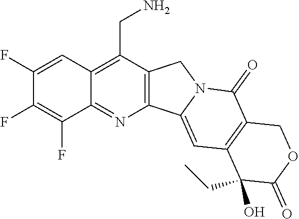 [SMILES: CC[C@@]1(O)C(=O)OCc2c1cc1n(c2=O)Cc2c-1nc1c(F)c(F)c(F)cc1c2CN] |
| 2aaa | 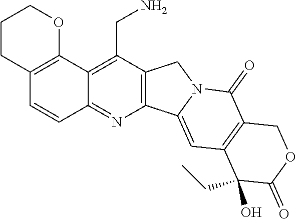 [SMILES: CC[C@@]1(O)C(=O)OCc2c1cc1n(c2=O)Cc2c-1nc1ccc3c(c1c2CN)OCCC3] |
| 2aab | 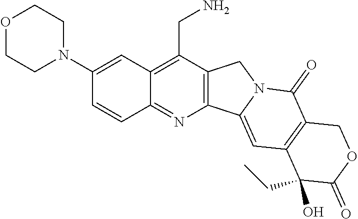 [SMILES: CC[C@@]1(O)C(=O)OCc2c1cc1n(c2=O)Cc2c-1nc1ccc(N3CCOCC3)cc1c2CN] |
| 2aac | 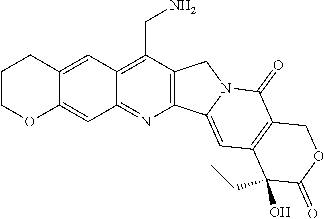 [SMILES: CC[C@@]1(O)C(=O)OCc2c1cc1n(c2=O)Cc2c-1nc1cc3c(cc1c2CN)CCCO3] |
| 3a | 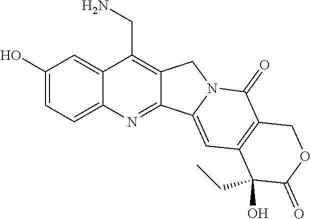 [SMILES: CC[C@@]1(O)C(=O)OCc2c1cc1n(c2=O)Cc2c-1nc1ccc(O)cc1c2CN] |
| 3b | 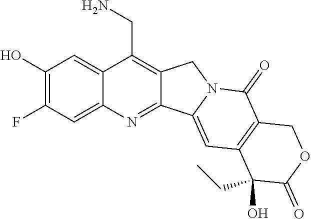 [SMILES: CC[C@@]1(O)C(=O)OCc2c1cc1n(c2=O)Cc2c-1nc1cc(F)c(O)cc1c2CN] |
| 3c | 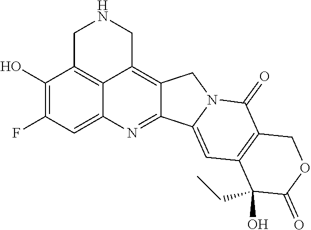 [SMILES: CC[C@@]1(O)C(=O)OCc2c1cc1n(c2=O)Cc2c-1nc1cc(F)c(O)c3c1c2CNC3] |
| 3d | 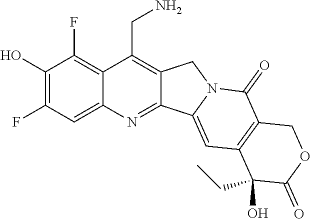 [SMILES: CC[C@@]1(O)C(=O)OCc2c1cc1n(c2=O)Cc2c-1nc1cc(F)c(O)c(F)c1c2CN] |
| 3e | 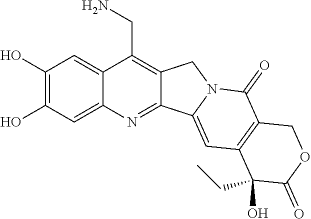 [SMILES: CC[C@@]1(O)C(=O)OCc2c1cc1n(c2=O)Cc2c-1nc1cc(O)c(O)cc1c2CN] |
| 5 | 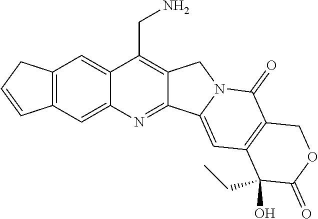 [SMILES: CC[C@@]1(O)C(=O)OCc2c1cc1n(c2=O)Cc2c-1nc1cc3c(cc1c2CN)CC=C3] |
| 5a | 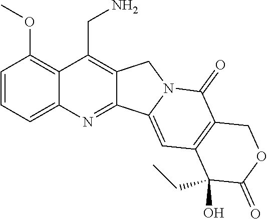 [SMILES: CC[C@@]1(O)C(=O)OCc2c1cc1n(c2=O)Cc2c-1nc1cccc(OC)c1c2CN] |
| 5b | 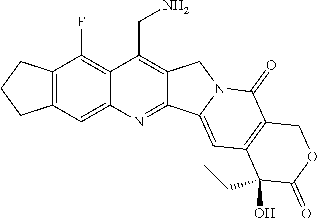 [SMILES: CC[C@@]1(O)C(=O)OCc2c1cc1n(c2=O)Cc2c-1nc1cc3c(c(F)c1c2CN)CCC3] |
| 5c | 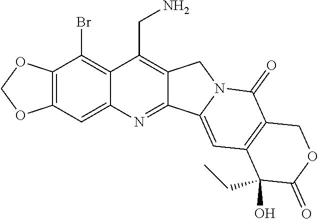 [SMILES: CC[C@@]1(O)C(=O)OCc2c1cc1n(c2=O)Cc2c-1nc1cc3c(c(Br)c1c2CN)OCO3] |
| 6 | 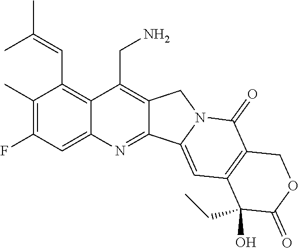 [SMILES: CC[C@@]1(O)C(=O)OCc2c1cc1n(c2=O)Cc2c-1nc1cc(F)c(C)c(C=C(C)C)c1c2CN] |
| 6a | 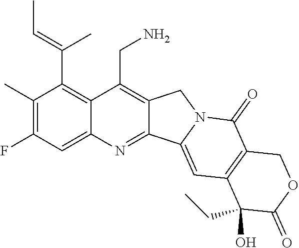 [SMILES: C/C=C(\C)c1c(C)c(F)cc2nc3c(c(CN)c12)Cn1c-3cc2c(c1=O)COC(=O)[C@]2(O)CC] |
| 6b | 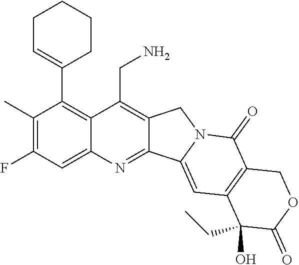 [SMILES: CC[C@@]1(O)C(=O)OCc2c1cc1n(c2=O)Cc2c-1nc1cc(F)c(C)c(C3=CCCCC3)c1c2CN] |
| 6c | 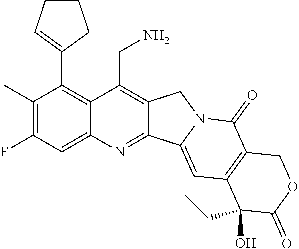 [SMILES: CC[C@@]1(O)C(=O)OCc2c1cc1n(c2=O)Cc2c-1nc1cc(F)c(C)c(C3=CCCC3)c1c2CN] |
| 6d | 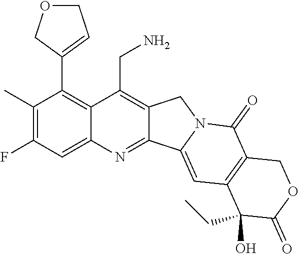 [SMILES: CC[C@@]1(O)C(=O)OCc2c1cc1n(c2=O)Cc2c-1nc1cc(F)c(C)c(C3=CCOC3)c1c2CN] |
| 6e | 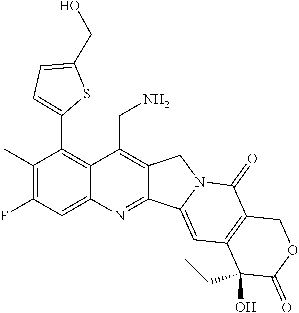 [SMILES: CC[C@@]1(O)C(=O)OCc2c1cc1n(c2=O)Cc2c-1nc1cc(F)c(C)c(-c3ccc(CO)s3)c1c2CN] |
| 6f | 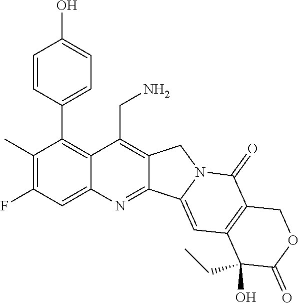 [SMILES: CC[C@@]1(O)C(=O)OCc2c1cc1n(c2=O)Cc2c-1nc1cc(F)c(C)c(-c3ccc(O)cc3)c1c2CN] |
| 6g | 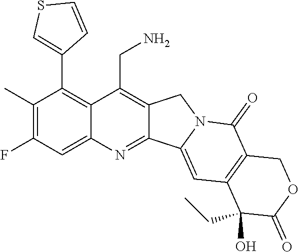 [SMILES: CC[C@@]1(O)C(=O)OCc2c1cc1n(c2=O)Cc2c-1nc1cc(F)c(C)c(-c3ccsc3)c1c2CN] |
| 6h | 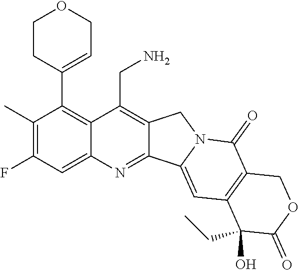 [SMILES: CC[C@@]1(O)C(=O)OCc2c1cc1n(c2=O)Cc2c-1nc1cc(F)c(C)c(C3=CCOCC3)c1c2CN] |
| 6i | 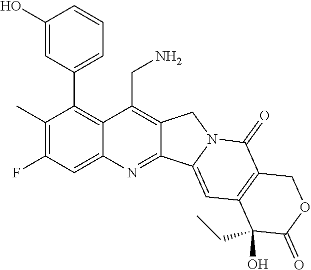 [SMILES: CC[C@@]1(O)C(=O)OCc2c1cc1n(c2=O)Cc2c-1nc1cc(F)c(C)c(-c3cccc(O)c3)c1c2CN] |
| 6j | 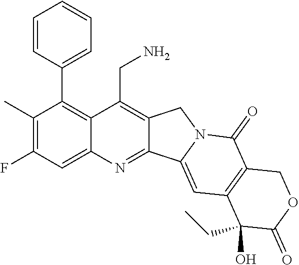 [SMILES: CC[C@@]1(O)C(=O)OCc2c1cc1n(c2=O)Cc2c-1nc1cc(F)c(C)c(-c3ccccc3)c1c2CN] |
| 6k | 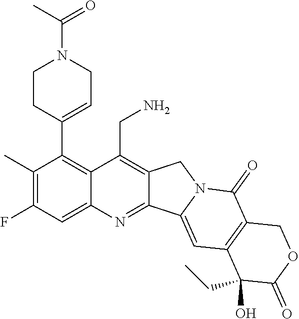 [SMILES: CC[C@@]1(O)C(=O)OCc2c1cc1n(c2=O)Cc2c-1nc1cc(F)c(C)c(C3=CCN(C(C)=O)CC3)c1c2CN] |
| 6l | 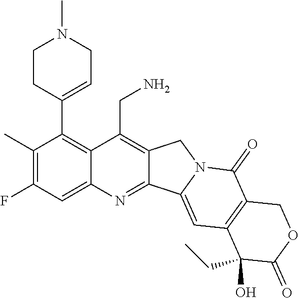 [SMILES: CC[C@@]1(O)C(=O)OCc2c1cc1n(c2=O)Cc2c-1nc1cc(F)c(C)c(C3=CCN(C)CC3)c1c2CN] |
| 6m | 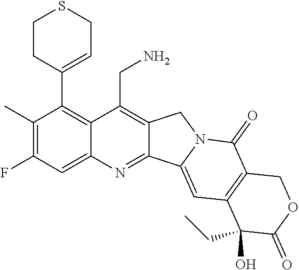 [SMILES: CC[C@@]1(O)C(=O)OCc2c1cc1n(c2=O)Cc2c-1nc1cc(F)c(C)c(C3=CCSCC3)c1c2CN] |
| 6n | 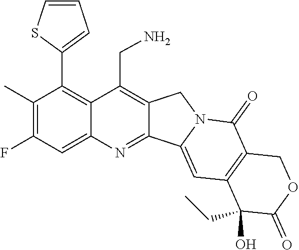 [SMILES: CC[C@@]1(O)C(=O)OCc2c1cc1n(c2=O)Cc2c-1nc1cc(F)c(C)c(-c3cccs3)c1c2CN] |
| 6o | 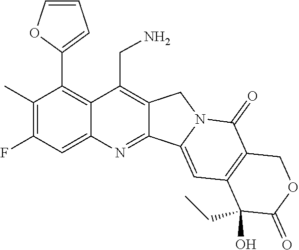 [SMILES: CC[C@@]1(O)C(=O)OCc2c1cc1n(c2=O)Cc2c-1nc1cc(F)c(C)c(-c3ccco3)c1c2CN] |
| 6p | 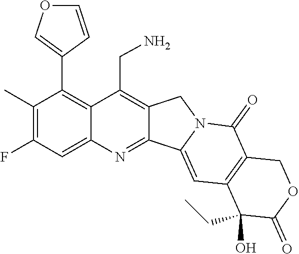 [SMILES: CC[C@@]1(O)C(=O)OCc2c1cc1n(c2=O)Cc2c-1nc1cc(F)c(C)c(-c3ccoc3)c1c2CN] |
| 33 | 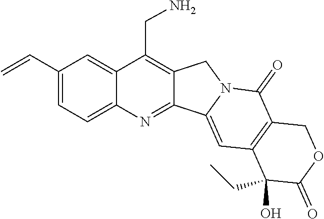 [SMILES: C=Cc1ccc2nc3c(c(CN)c2c1)Cn1c-3cc2c(c1=O)COC(=O)[C@]2(O)CC] |
| 33a | 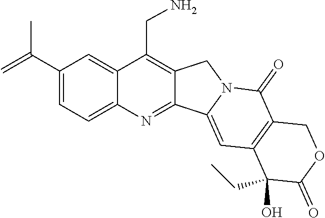 [SMILES: C=C(C)c1ccc2nc3c(c(CN)c2c1)Cn1c-3cc2c(c1=O)COC(=O)[C@]2(O)CC] |
| 11 (R) | 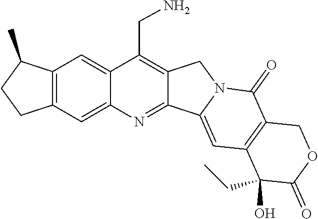 [SMILES: CC[C@@]1(O)C(=O)OCc2c1cc1n(c2=O)Cc2c-1nc1cc3c(cc1c2CN)[C@H](C)CC3] |
| 11 (S) | 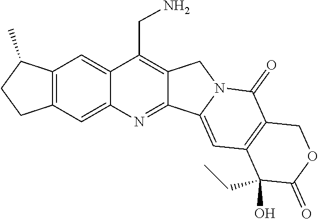 [SMILES: CC[C@@]1(O)C(=O)OCc2c1cc1n(c2=O)Cc2c-1nc1cc3c(cc1c2CN)[C@@H](C)CC3] |
| 10 | 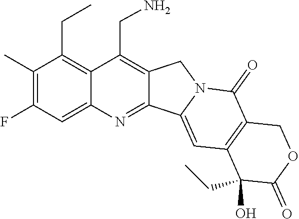 [SMILES: CCc1c(C)c(F)cc2nc3c(c(CN)c12)Cn1c-3cc2c(c1=O)COC(=O)[C@]2(O)CC] |
| 12 | 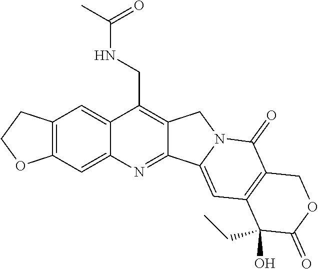 [SMILES: CC[C@@]1(O)C(=O)OCc2c1cc1n(c2=O)Cc2c-1nc1cc3c(cc1c2CNC(C)=O)CCO3] |
| 12a | 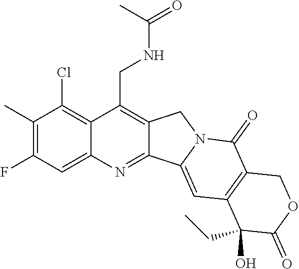 [SMILES: CC[C@@]1(O)C(=O)OCc2c1cc1n(c2=O)Cc2c-1nc1cc(F)c(C)c(Cl)c1c2CNC(C)=O] |
| 12b | 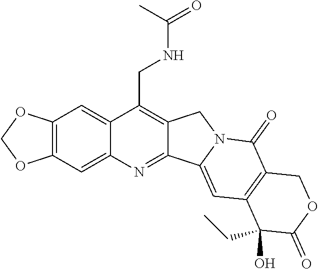 [SMILES: CC[C@@]1(O)C(=O)OCc2c1cc1n(c2=O)Cc2c-1nc1cc3c(cc1c2CNC(C)=O)OCO3] |
| 13 | 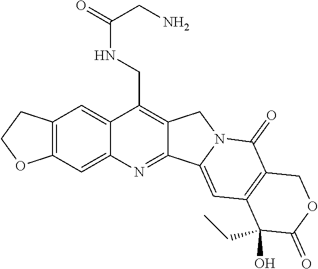 [SMILES: CC[C@@]1(O)C(=O)OCc2c1cc1n(c2=O)Cc2c-1nc1cc3c(cc1c2CNC(=O)CN)CCO3] |
| 13a | 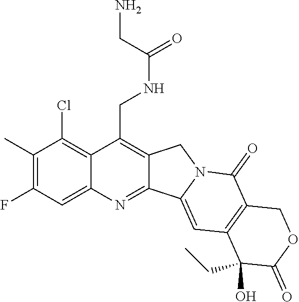 [SMILES: CC[C@@]1(O)C(=O)OCc2c1cc1n(c2=O)Cc2c-1nc1cc(F)c(C)c(Cl)c1c2CNC(=O)CN] |
| 13b | 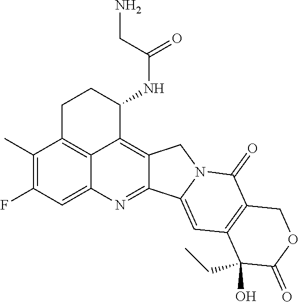 [SMILES: CC[C@@]1(O)C(=O)OCc2c1cc1n(c2=O)Cc2c-1nc1cc(F)c(C)c3c1c2[C@@H](NC(=O)CN)CC3] |
| 7 | 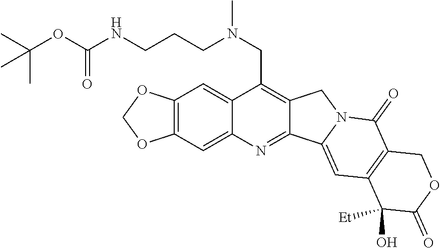 [SMILES: CC[C@@]1(O)C(=O)OCc2c1cc1n(c2=O)Cc2c-1nc1cc3c(cc1c2CN(C)CCCNC(=O)OC(C)(C)C)OCO3] |
| 7a | 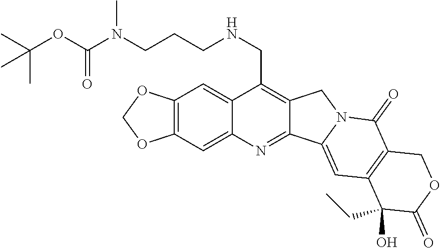 [SMILES: CC[C@@]1(O)C(=O)OCc2c1cc1n(c2=O)Cc2c-1nc1cc3c(cc1c2CNCCCN(C)C(=O)OC(C)(C)C)OCO3] |
| 7b | 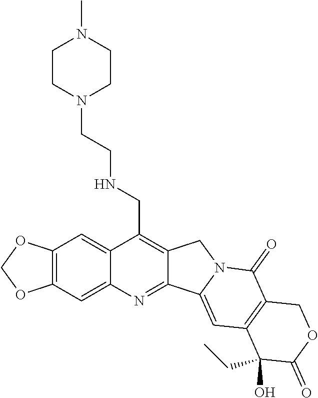 [SMILES: CC[C@@]1(O)C(=O)OCc2c1cc1n(c2=O)Cc2c-1nc1cc3c(cc1c2CNCCN1CCN(C)CC1)OCO3] |
| 7c | 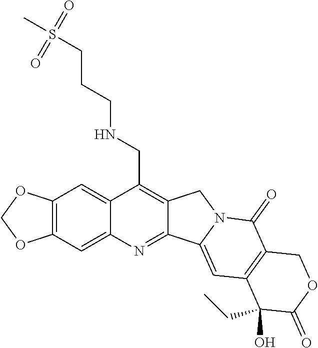 [SMILES: CC[C@@]1(O)C(=O)OCc2c1cc1n(c2=O)Cc2c-1nc1cc3c(cc1c2CNCCCS(C)(=O)=O)OCO3] |
| 7d | 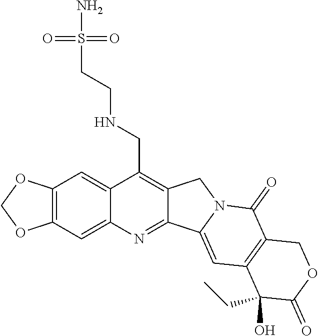 [SMILES: CC[C@@]1(O)C(=O)OCc2c1cc1n(c2=O)Cc2c-1nc1cc3c(cc1c2CNCCS(N)(=O)=O)OCO3] |
| 7e | 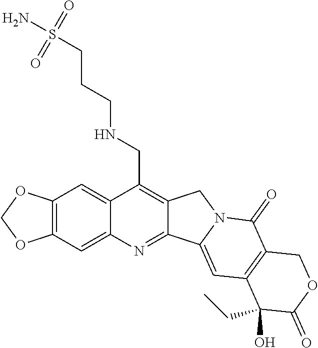 [SMILES: CC[C@@]1(O)C(=O)OCc2c1cc1n(c2=O)Cc2c-1nc1cc3c(cc1c2CNCCCS(N)(=O)=O)OCO3] |
| 7f | 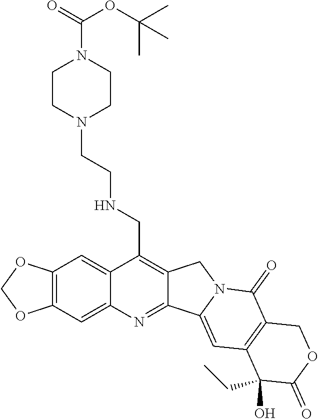 [SMILES: CC[C@@]1(O)C(=O)OCc2c1cc1n(c2=O)Cc2c-1nc1cc3c(cc1c2CNCCN1CCN(C(=O)OC(C)(C)C)CC1)OCO3] |
| 7g | 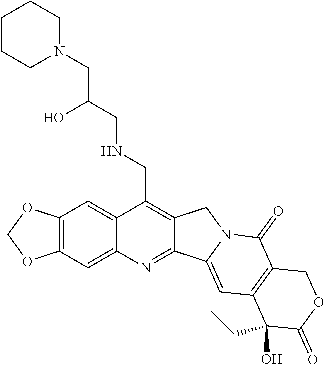 [SMILES: CC[C@@]1(O)C(=O)OCc2c1cc1n(c2=O)Cc2c-1nc1cc3c(cc1c2CNCC(O)CN1CCCCC1)OCO3] |
| 7h | 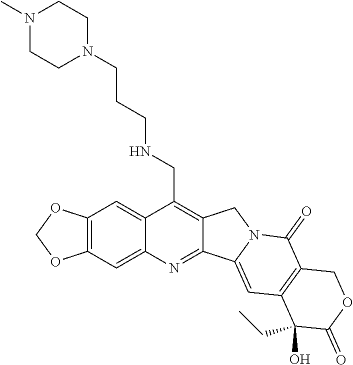 [SMILES: CC[C@@]1(O)C(=O)OCc2c1cc1n(c2=O)Cc2c-1nc1cc3c(cc1c2CNCCCN1CCN(C)CC1)OCO3] |
| 7i | 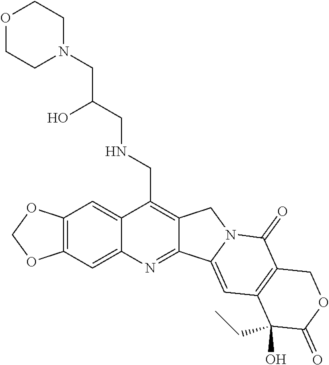 [SMILES: CC[C@@]1(O)C(=O)OCc2c1cc1n(c2=O)Cc2c-1nc1cc3c(cc1c2CNCC(O)CN1CCOCC1)OCO3] |
| 7j | 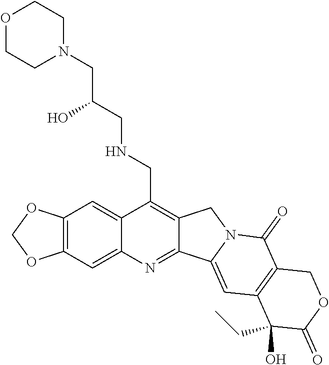 [SMILES: CC[C@@]1(O)C(=O)OCc2c1cc1n(c2=O)Cc2c-1nc1cc3c(cc1c2CNC[C@H](O)CN1CCOCC1)OCO3] |
| 7k | 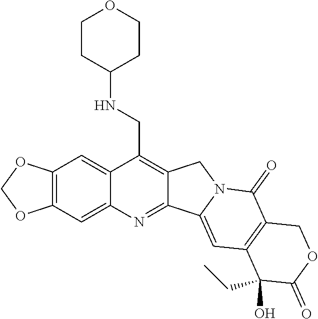 [SMILES: CC[C@@]1(O)C(=O)OCc2c1cc1n(c2=O)Cc2c-1nc1cc3c(cc1c2CNC1CCOCC1)OCO3] |
| 7l | 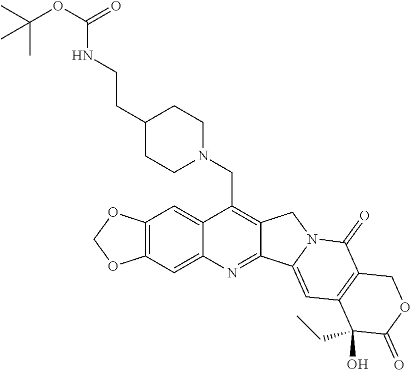 [SMILES: CC[C@@]1(O)C(=O)OCc2c1cc1n(c2=O)Cc2c-1nc1cc3c(cc1c2CN1CCC(CCNC(=O)OC(C)(C)C)CC1)OCO3] |
| 7m | 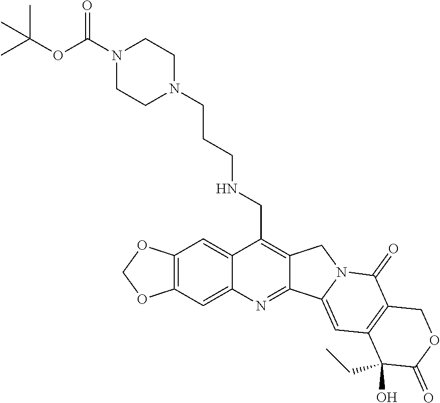 [SMILES: CC[C@@]1(O)C(=O)OCc2c1cc1n(c2=O)Cc2c-1nc1cc3c(cc1c2CNCCCN1CCN(C(=O)OC(C)(C)C)CC1)OCO3] |
| 7n | 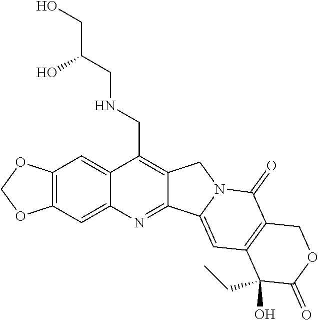 [SMILES: CC[C@@]1(O)C(=O)OCc2c1cc1n(c2=O)Cc2c-1nc1cc3c(cc1c2CNC[C@H](O)CO)OCO3] |
| 7o | 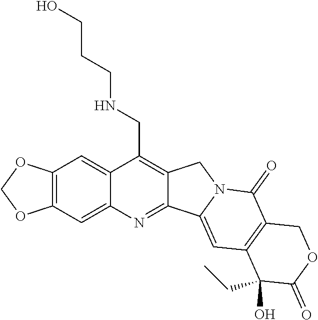 [SMILES: CC[C@@]1(O)C(=O)OCc2c1cc1n(c2=O)Cc2c-1nc1cc3c(cc1c2CNCCCO)OCO3] |
| 7p | 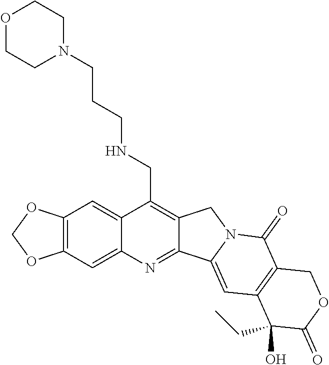 [SMILES: CC[C@@]1(O)C(=O)OCc2c1cc1n(c2=O)Cc2c-1nc1cc3c(cc1c2CNCCCN1CCOCC1)OCO3] |
| 7q | 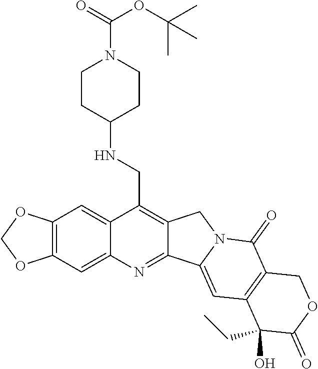 [SMILES: CC[C@@]1(O)C(=O)OCc2c1cc1n(c2=O)Cc2c-1nc1cc3c(cc1c2CNC1CCN(C(=O)OC(C)(C)C)CC1)OCO3] |
| 7r | 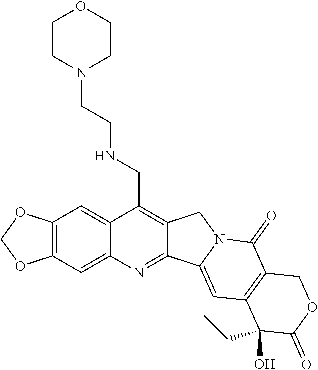 [SMILES: CC[C@@]1(O)C(=O)OCc2c1cc1n(c2=O)Cc2c-1nc1cc3c(cc1c2CNCCN1CCOCC1)OCO3] |
| 7s | 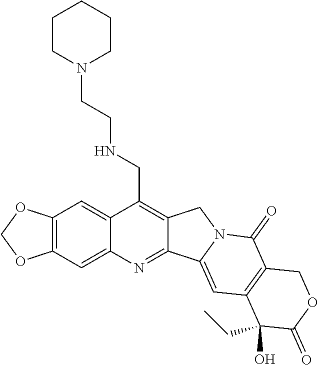 [SMILES: CC[C@@]1(O)C(=O)OCc2c1cc1n(c2=O)Cc2c-1nc1cc3c(cc1c2CNCCN1CCCCC1)OCO3] |
| 7t | 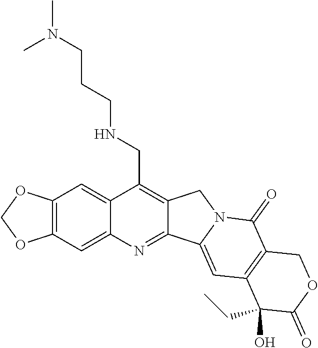 [SMILES: CC[C@@]1(O)C(=O)OCc2c1cc1n(c2=O)Cc2c-1nc1cc3c(cc1c2CNCCCN(C)C)OCO3] |
| 7u | 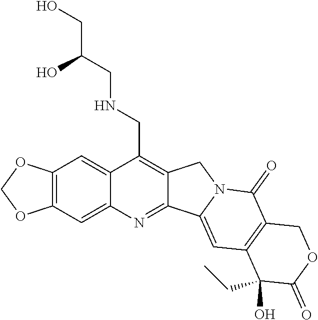 [SMILES: CC[C@@]1(O)C(=O)OCc2c1cc1n(c2=O)Cc2c-1nc1cc3c(cc1c2CNC[C@@H](O)CO)OCO3] |
| 7v | 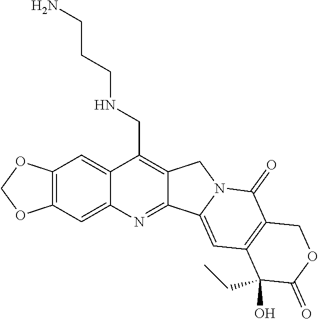 [SMILES: CC[C@@]1(O)C(=O)OCc2c1cc1n(c2=O)Cc2c-1nc1cc3c(cc1c2CNCCCN)OCO3] |
| 7w | 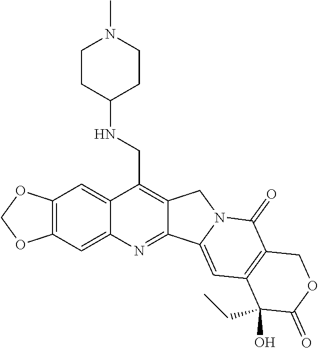 [SMILES: CC[C@@]1(O)C(=O)OCc2c1cc1n(c2=O)Cc2c-1nc1cc3c(cc1c2CNC1CCN(C)CC1)OCO3] |
| 7x | 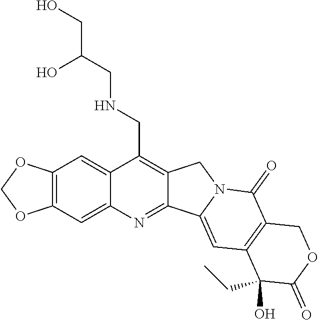 [SMILES: CC[C@@]1(O)C(=O)OCc2c1cc1n(c2=O)Cc2c-1nc1cc3c(cc1c2CNCC(O)CO)OCO3] |
| 7y | 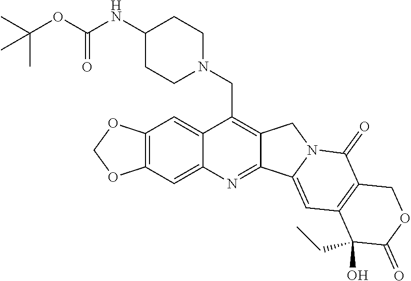 [SMILES: CC[C@@]1(O)C(=O)OCc2c1cc1n(c2=O)Cc2c-1nc1cc3c(cc1c2CN1CCC(NC(=O)OC(C)(C)C)CC1)OCO3] |
| 7z | 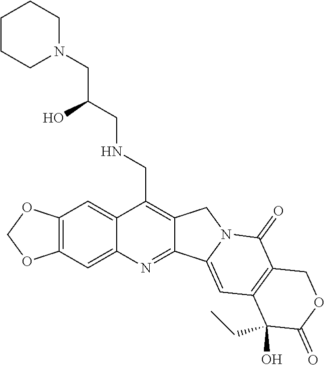 [SMILES: CC[C@@]1(O)C(=O)OCc2c1cc1n(c2=O)Cc2c-1nc1cc3c(cc1c2CNC[C@@H](O)CN1CCCCC1)OCO3] |
| 7aa | 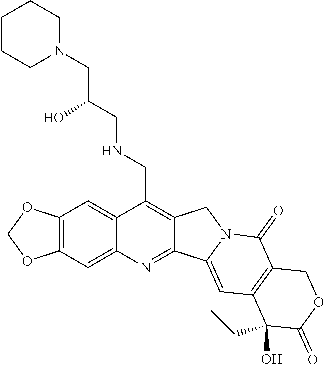 [SMILES: CC[C@@]1(O)C(=O)OCc2c1cc1n(c2=O)Cc2c-1nc1cc3c(cc1c2CNC[C@H](O)CN1CCCCC1)OCO3] |
| 7ab | 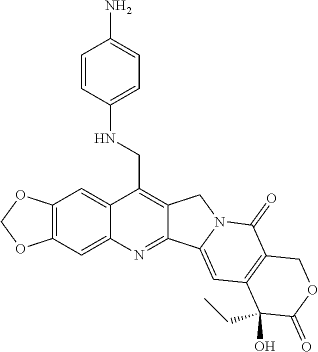 [SMILES: CC[C@@]1(O)C(=O)OCc2c1cc1n(c2=O)Cc2c-1nc1cc3c(cc1c2CNc1ccc(N)cc1)OCO3] |
| 7ac | 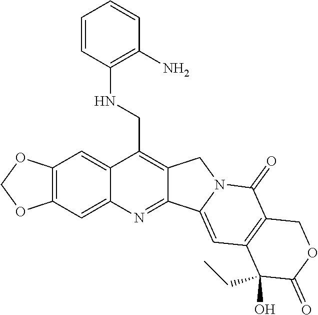 [SMILES: CC[C@@]1(O)C(=O)OCc2c1cc1n(c2=O)Cc2c-1nc1cc3c(cc1c2CNc1ccccc1N)OCO3] |
| 7ad | 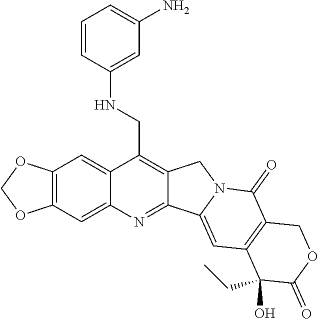 [SMILES: CC[C@@]1(O)C(=O)OCc2c1cc1n(c2=O)Cc2c-1nc1cc3c(cc1c2CNc1cccc(N)c1)OCO3] |
| 7ae | 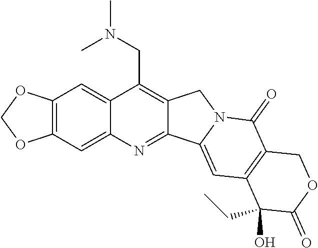 [SMILES: CC[C@@]1(O)C(=O)OCc2c1cc1n(c2=O)Cc2c-1nc1cc3c(cc1c2CN(C)C)OCO3] |
| 7af | 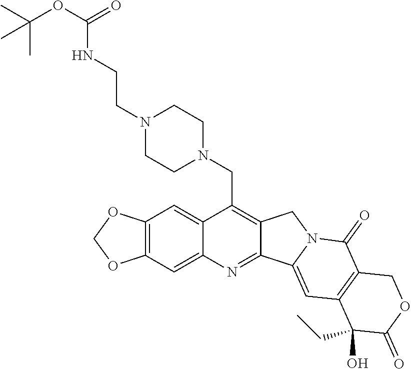 [SMILES: CC[C@@]1(O)C(=O)OCc2c1cc1n(c2=O)Cc2c-1nc1cc3c(cc1c2CN1CCN(CCNC(=O)OC(C)(C)C)CC1)OCO3] |
| 7ag | 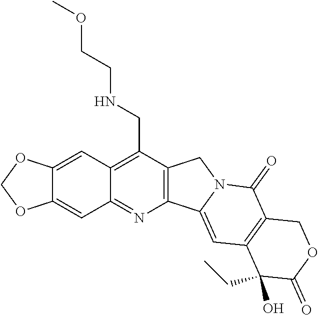 [SMILES: CC[C@@]1(O)C(=O)OCc2c1cc1n(c2=O)Cc2c-1nc1cc3c(cc1c2CNCCOC)OCO3] |
| 7ah | 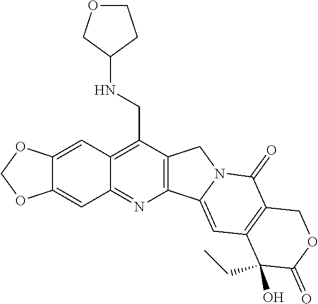 [SMILES: CC[C@@]1(O)C(=O)OCc2c1cc1n(c2=O)Cc2c-1nc1cc3c(cc1c2CNC1CCOC1)OCO3] |
| 7ai | 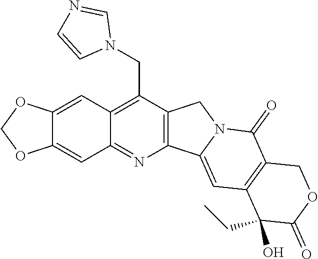 [SMILES: CC[C@@]1(O)C(=O)OCc2c1cc1n(c2=O)Cc2c-1nc1cc3c(cc1c2Cn1ccnc1)OCO3] |
| 7aj | 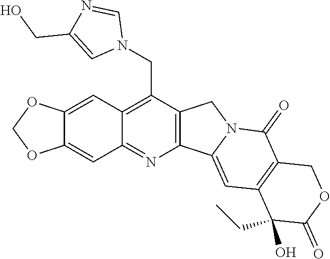 [SMILES: CC[C@@]1(O)C(=O)OCc2c1cc1n(c2=O)Cc2c-1nc1cc3c(cc1c2Cn1cnc(CO)c1)OCO3] |
| 7ak | 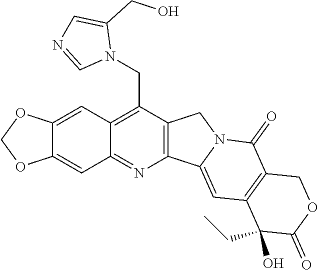 [SMILES: CC[C@@]1(O)C(=O)OCc2c1cc1n(c2=O)Cc2c-1nc1cc3c(cc1c2Cn1cncc1CO)OCO3] |
| 7al | 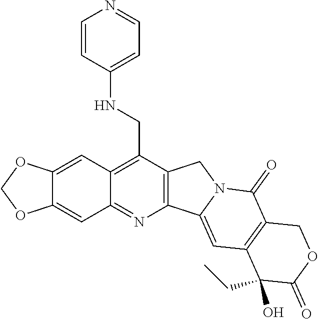 [SMILES: CC[C@@]1(O)C(=O)OCc2c1cc1n(c2=O)Cc2c-1nc1cc3c(cc1c2CNc1ccncc1)OCO3] |
| 7am | 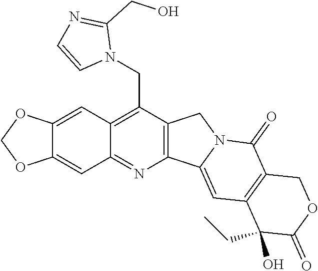 [SMILES: CC[C@@]1(O)C(=O)OCc2c1cc1n(c2=O)Cc2c-1nc1cc3c(cc1c2Cn1ccnc1CO)OCO3] |
| 7an | 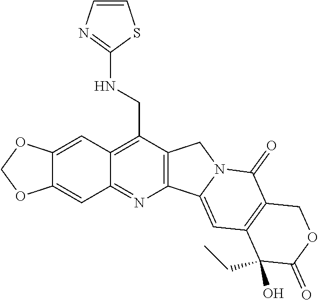 [SMILES: CC[C@@]1(O)C(=O)OCc2c1cc1n(c2=O)Cc2c-1nc1cc3c(cc1c2CNc1nccs1)OCO3] |
| 34 | 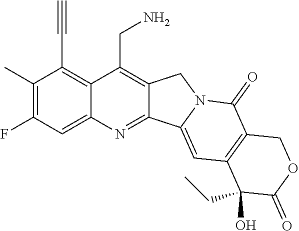 [SMILES: C#Cc1c(C)c(F)cc2nc3c(c(CN)c12)Cn1c-3cc2c(c1=O)COC(=O)[C@]2(O)CC] |
| 8 | 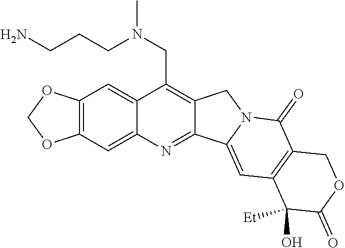 [SMILES: CC[C@@]1(O)C(=O)OCc2c1cc1n(c2=O)Cc2c-1nc1cc3c(cc1c2CN(C)CCCN)OCO3] |
| 8a | 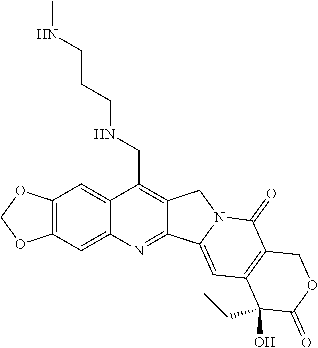 [SMILES: CC[C@@]1(O)C(=O)OCc2c1cc1n(c2=O)Cc2c-1nc1cc3c(cc1c2CNCCCNC)OCO3] |
| 8b | 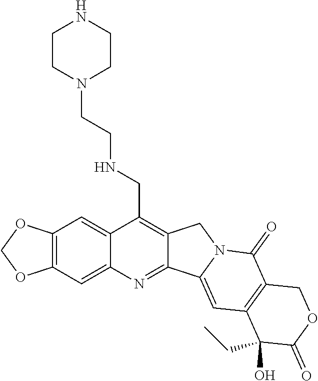 [SMILES: CC[C@@]1(O)C(=O)OCc2c1cc1n(c2=O)Cc2c-1nc1cc3c(cc1c2CNCCN1CCNCC1)OCO3] |
| 8c | 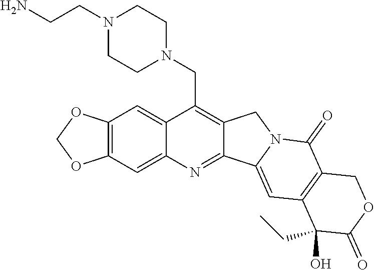 [SMILES: CC[C@@]1(O)C(=O)OCc2c1cc1n(c2=O)Cc2c-1nc1cc3c(cc1c2CN1CCN(CCN)CC1)OCO3] |
| 8d | 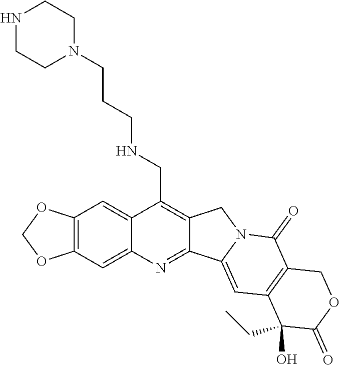 [SMILES: CC[C@@]1(O)C(=O)OCc2c1cc1n(c2=O)Cc2c-1nc1cc3c(cc1c2CNCCCN1CCNCC1)OCO3] |
| 8e | 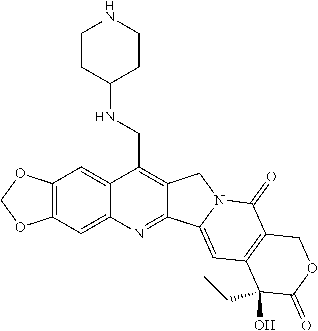 [SMILES: CC[C@@]1(O)C(=O)OCc2c1cc1n(c2=O)Cc2c-1nc1cc3c(cc1c2CNC1CCNCC1)OCO3] |
| 8f | 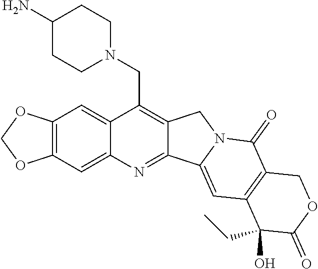 [SMILES: CC[C@@]1(O)C(=O)OCc2c1cc1n(c2=O)Cc2c-1nc1cc3c(cc1c2CN1CCC(N)CC1)OCO3] |
| 9 | 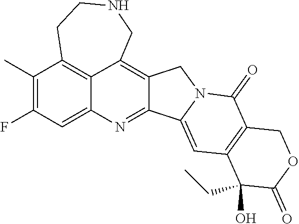 [SMILES: CC[C@@]1(O)C(=O)OCc2c1cc1n(c2=O)Cc2c-1nc1cc(F)c(C)c3c1c2CNCC3] |
| 9a | 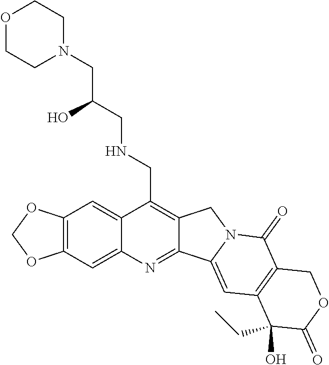 [SMILES: CC[C@@]1(O)C(=O)OCc2c1cc1n(c2=O)Cc2c-1nc1cc3c(cc1c2CNC[C@@H](O)CN1CCOCC1)OCO3] |
| 9b | 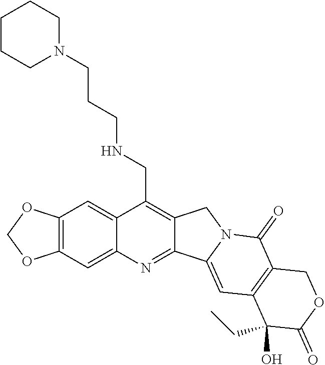 [SMILES: CC[C@@]1(O)C(=O)OCc2c1cc1n(c2=O)Cc2c-1nc1cc3c(cc1c2CNCCCN1CCCCC1)OCO3] |
| 9c | 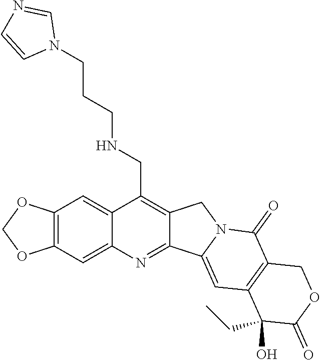 [SMILES: CC[C@@]1(O)C(=O)OCc2c1cc1n(c2=O)Cc2c-1nc1cc3c(cc1c2CNCCCn1ccnc1)OCO3] |
| 9d | 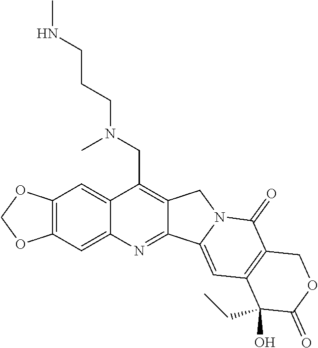 [SMILES: CC[C@@]1(O)C(=O)OCc2c1cc1n(c2=O)Cc2c-1nc1cc3c(cc1c2CN(C)CCCNC)OCO3] |
| 9e | 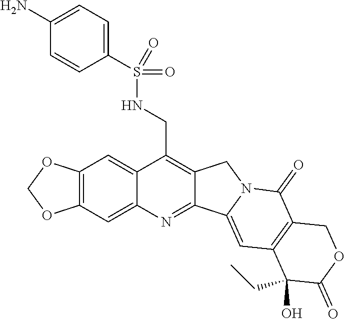 [SMILES: CC[C@@]1(O)C(=O)OCc2c1cc1n(c2=O)Cc2c-1nc1cc3c(cc1c2CNS(=O)(=O)c1ccc(N)cc1)OCO3] |
| 9f | 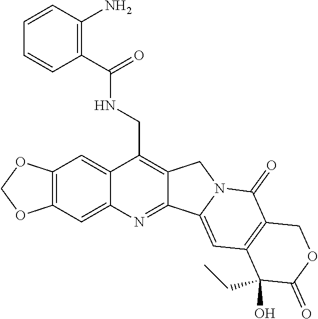 [SMILES: CC[C@@]1(O)C(=O)OCc2c1cc1n(c2=O)Cc2c-1nc1cc3c(cc1c2CNC(=O)c1ccccc1N)OCO3] |
| 9g | 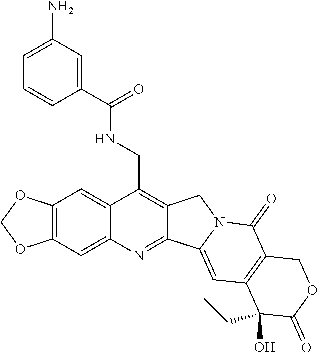 [SMILES: CC[C@@]1(O)C(=O)OCc2c1cc1n(c2=O)Cc2c-1nc1cc3c(cc1c2CNC(=O)c1cccc(N)c1)OCO3] |
| 9h | 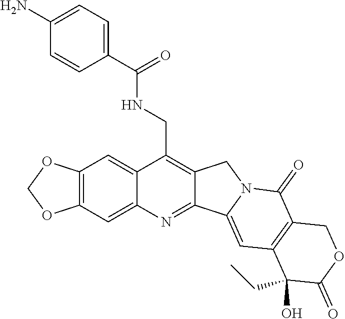 [SMILES: CC[C@@]1(O)C(=O)OCc2c1cc1n(c2=O)Cc2c-1nc1cc3c(cc1c2CNC(=O)c1ccc(N)cc1)OCO3] |
| 9i | 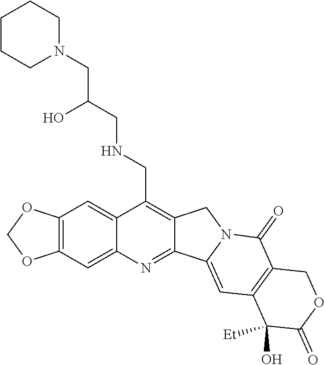 [SMILES: CC[C@@]1(O)C(=O)OCc2c1cc1n(c2=O)Cc2c-1nc1cc3c(cc1c2CNCC(O)CN1CCCCC1)OCO3] |
| 9j | 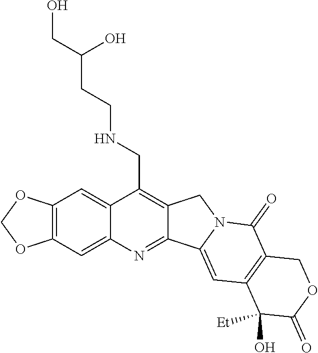 [SMILES: CC[C@@]1(O)C(=O)OCc2c1cc1n(c2=O)Cc2c-1nc1cc3c(cc1c2CNCCC(O)CO)OCO3] |
| 9k | 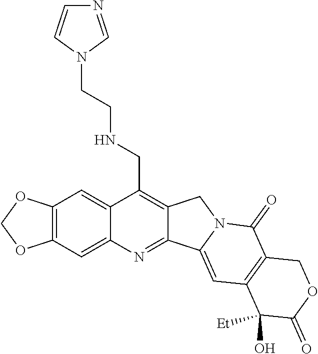 [SMILES: CC[C@@]1(O)C(=O)OCc2c1cc1n(c2=O)Cc2c-1nc1cc3c(cc1c2CNCCn1ccnc1)OCO3] |
| 9l | 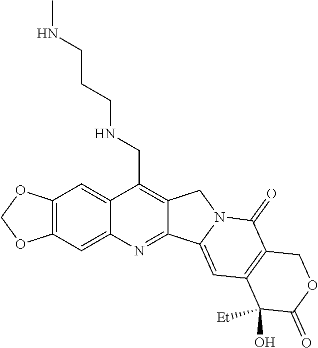 [SMILES: CC[C@@]1(O)C(=O)OCc2c1cc1n(c2=O)Cc2c-1nc1cc3c(cc1c2CNCCCNC)OCO3] |
| 9m | 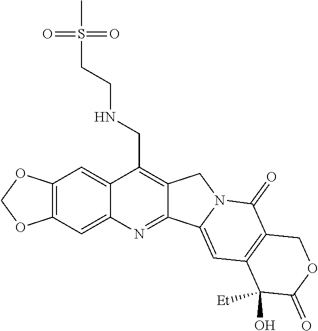 [SMILES: CC[C@@]1(O)C(=O)OCc2c1cc1n(c2=O)Cc2c-1nc1cc3c(cc1c2CNCCS(C)(=O)=O)OCO3] |
| 35 | 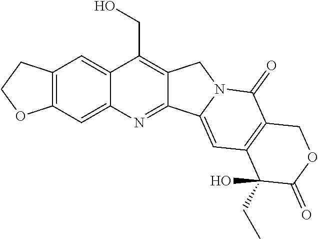 [SMILES: CC[C@@]1(O)C(=O)OCc2c1cc1n(c2=O)Cc2c-1nc1cc3c(cc1c2CO)CCO3] |
| 15a | 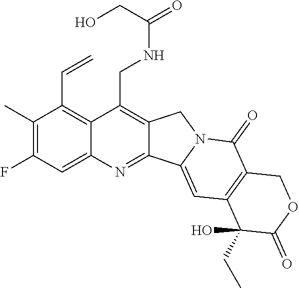 [SMILES: C=Cc1c(C)c(F)cc2nc3c(c(CNC(=O)CO)c12)Cn1c-3cc2c(c1=O)COC(=O)[C@]2(O)CC] |
| 15b | 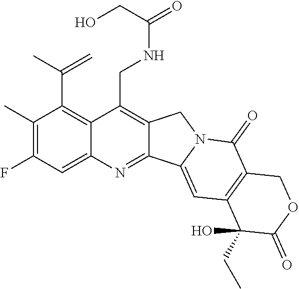 [SMILES: C=C(C)c1c(C)c(F)cc2nc3c(c(CNC(=O)CO)c12)Cn1c-3cc2c(c1=O)COC(=O)[C@]2(O)CC] |
| 15c | 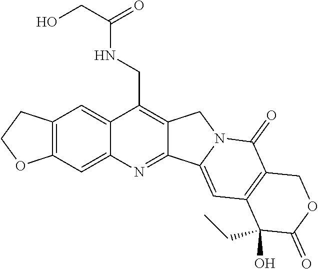 [SMILES: CC[C@@]1(O)C(=O)OCc2c1cc1n(c2=O)Cc2c-1nc1cc3c(cc1c2CNC(=O)CO)CCO3] |
| 15d | 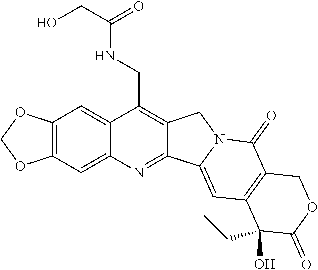 [SMILES: CC[C@@]1(O)C(=O)OCc2c1cc1n(c2=O)Cc2c-1nc1cc3c(cc1c2CNC(=O)CO)OCO3] |
| 16 | 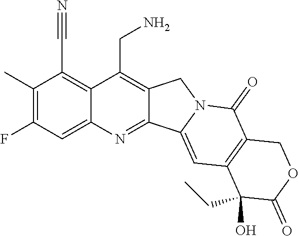 [SMILES: CC[C@@]1(O)C(=O)OCc2c1cc1n(c2=O)Cc2c-1nc1cc(F)c(C)c(C#N)c1c2CN] |
| 17 | 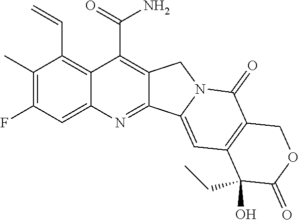 [SMILES: C=Cc1c(C)c(F)cc2nc3c(c(C(N)=O)c12)Cn1c-3cc2c(c1=O)COC(=O)[C@]2(O)CC] |

In one group of embodiments, Q has a formula selected from the group consisting of:
Z-A-RL- and -Z-A-RL-Y-,
   wherein RL is a Releasable Linker that is a Glycoside (e.g., Glucuronide) Unit and the groups Z, A and Y have the meanings provided above and in any one of the embodiments specifically recited herein.

In one group of embodiments, Q has a formula selected from the group consisting of:
Z-A-S*-RL- and -Z-A-S*-RL-Y-,
   wherein RL is a Releasable Linker that is a Glycoside (e.g., Glucuronide) Unit and the groups Z, A, S* and Y have the meanings provided above and in any one of the embodiments specifically recited herein.

In one group of embodiments, Q has a formula selected from the group consisting of:
Z-A-B(S*)-RL- and -Z-A-B(S*)-RL-Y-,
   wherein RL is a Releasable Linker that is a Glycoside (e.g., Glucuronide) Unit and the groups Z, A, S*, B and Y have the meanings provided above and in any one of the embodiments specifically recited herein.

In another group of embodiments, Q has a formula selected from the group consisting of:
-Z-A- or -Z-A-RL-,
   wherein RL is a Releasable Linker that is other than a Glycoside (e.g., Glucuronide) Unit and the groups Z and A have the meanings provided above and in any one of the embodiments specifically recited herein.

In another group of embodiments, Q has a formula selected from the group consisting of:
Z-A-S*-RL- and -Z-A-B(S*)-RL-,
   wherein RL is a Releasable Linker that is other than a Glycoside (e.g., Glucuronide) Unit and the groups Z, A, S* and B have the meanings provided above and in any one of the embodiments specifically recited herein.

In another group of embodiments, Q has a formula selected from the group consisting of:
Z-A-S*-W- and -Z-A-B(S*)-W-,
   wherein the groups Z, A, S*, B and W have the meanings provided above and in any one of the embodiments specifically recited herein.

In another group of embodiments, Q has a formula selected from the group consisting of:
Z-A-S*-W-RL- and -Z-A-B(S*)-W-RL-,
   wherein RL is a Releasable Linker that is other than a Glycoside (e.g., Glucuronide) Unit and the groups Z, A, S*, B and W have the meanings provided above and in any one of the embodiments specifically recited herein.

In one group of embodiments, the Camptothecin Conjugates in which Q has the formula of -Z-A-RL-, -Z-A-RL-Y-, -Z-A-S*-RL-, -Z-A-S*-RL-Y-, -Z-A-B(S*)-RL- or -Z-A-B(S*)-RL-Y- and are comprised of a Drug Unit having formula D₁ are represented by formulae of:

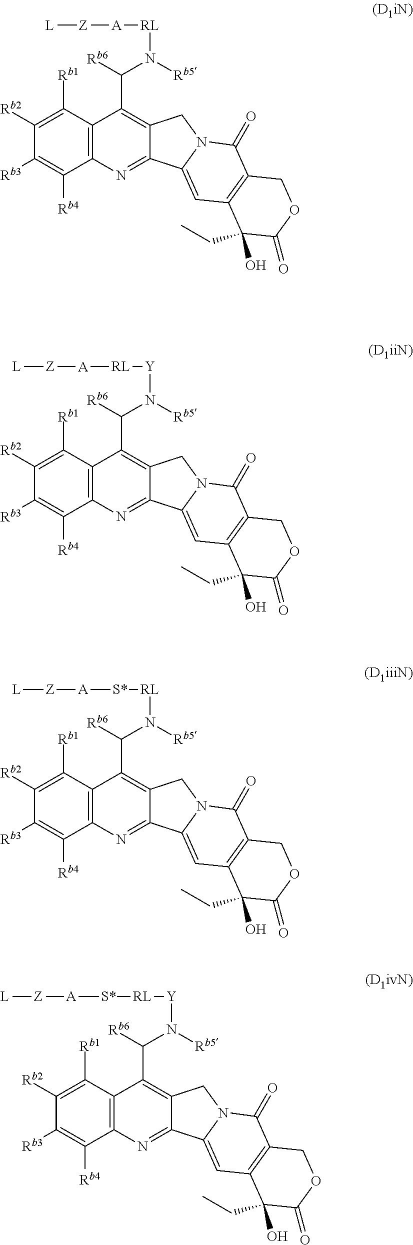

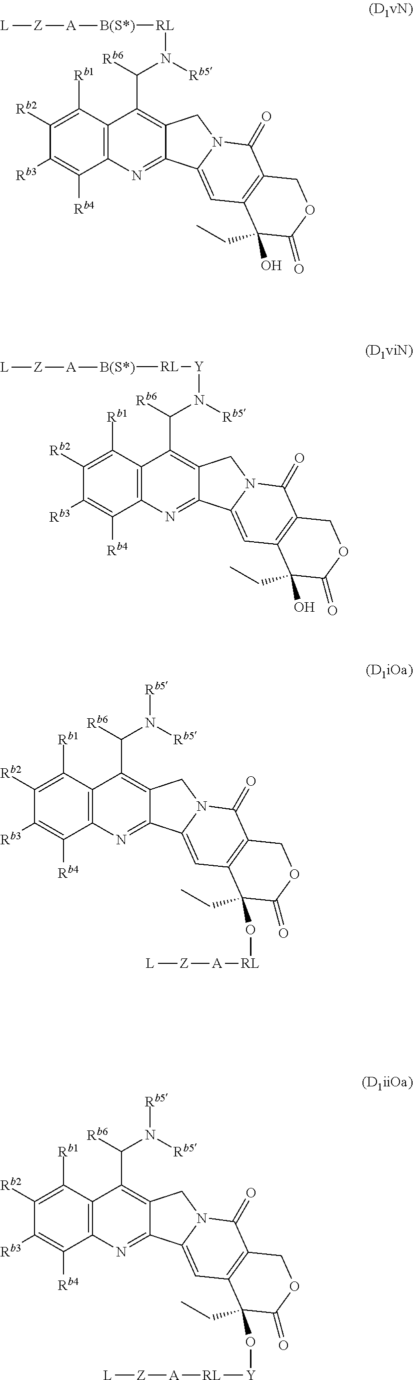

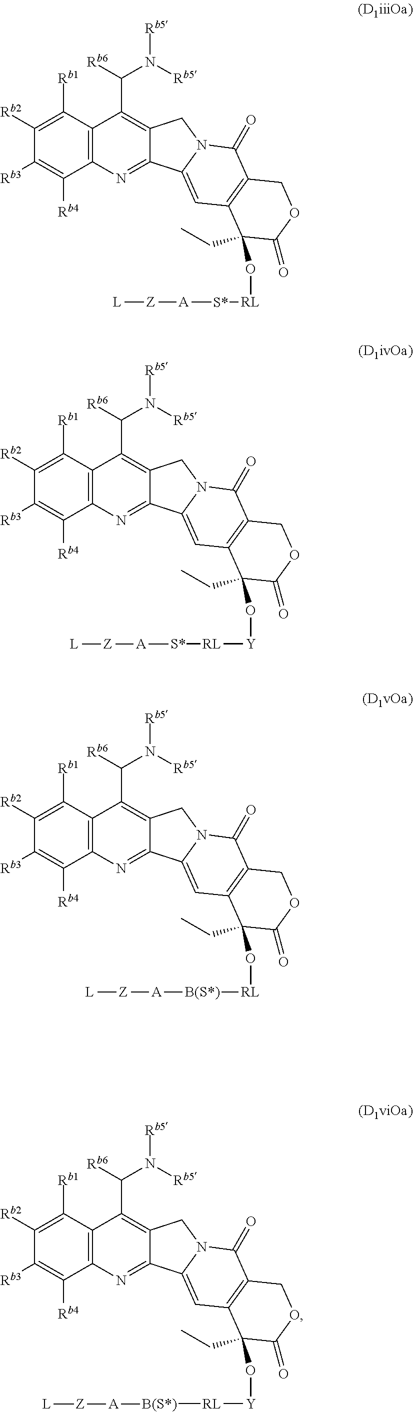

respectively, wherein RL is any one of the Releasable Linkers disclosed herein, preferably RL is a Glycoside (e.g., Glucuronide) Unit, and the groups L, Z, A, S*, B and Y have the meanings provided above and in any one of the embodiments specifically recited herein. Also provided herein are Camptothecin Conjugates corresponding to any of formulas D₁iN, D₁iiN, D₁iiiN, D₁ivN, D₁vN, or D₁viN wherein the nitrogen atom to which R^{x}′; is bound is replaced by an oxygen atom and R^{x}′; is absent, such that Q is attached to the Drug Unit via an oxygen atom of the Drug Unit.

In other embodiments the Camptothecin Conjugates in which Q has the formula of -Z-A-, -Z-A-RL-, -Z-A-S*-W-, -Z-A-B(S*)-W-, -Z-A-S*-RL-, -Z-A-B(S*)-RL-, -Z-A-S*-W-RL- and -Z-A-B(S*)-W-RL- and are comprised of a Drug Unit having formula D₁ are represented by formulae of:

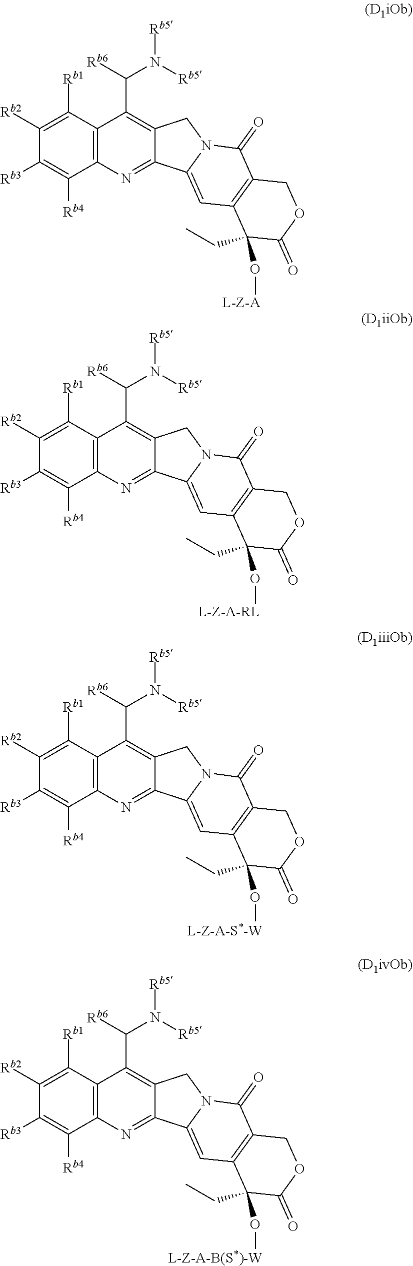

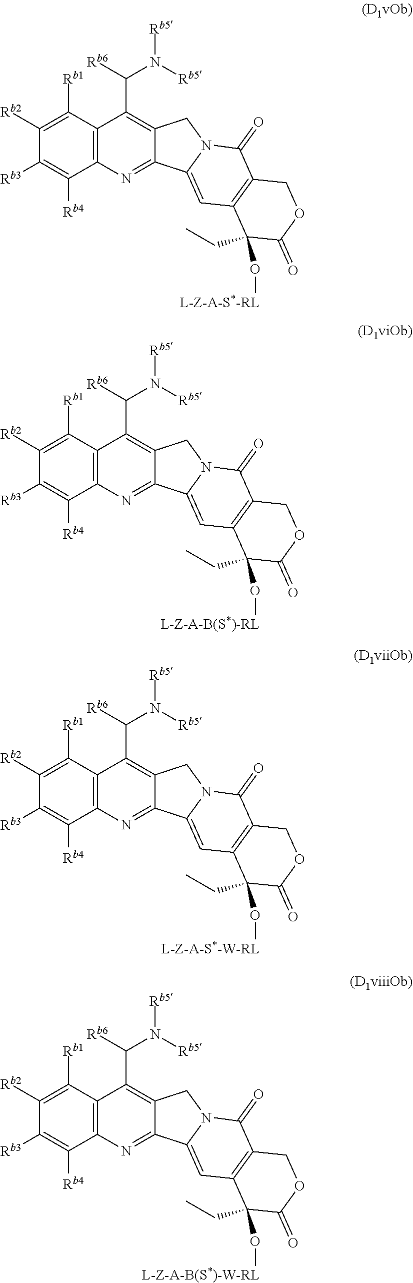

respectively, wherein RL is a Releasable Linker that is other than a Glycoside (e.g., Glucuronide) Unit and the groups L, Z, A, S*, B and W have the meanings provided above and in any one of the embodiments specifically recited herein.

In another group of embodiments, the Camptothecin Conjugates comprised of a Drug Unit having formula D1-I are represented by the formulae of:

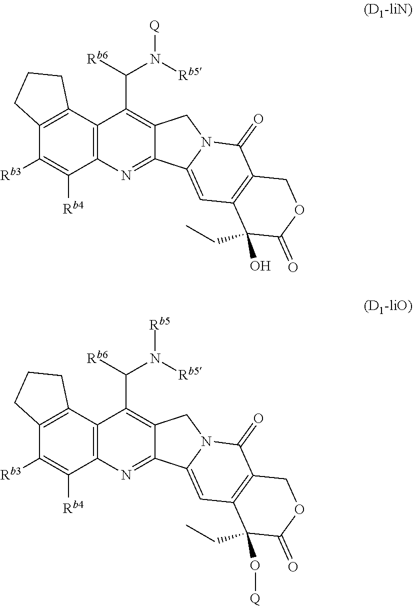

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁EN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-II are represented by the formulae of:

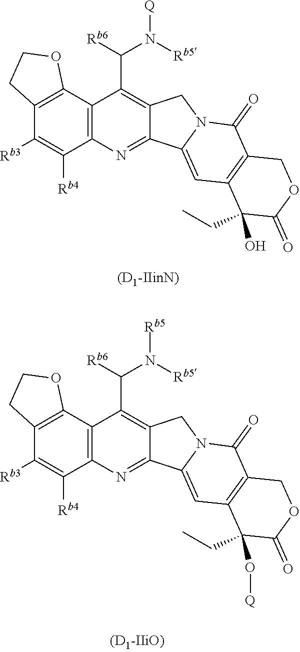

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁₁IiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-III are represented by the formulae of:

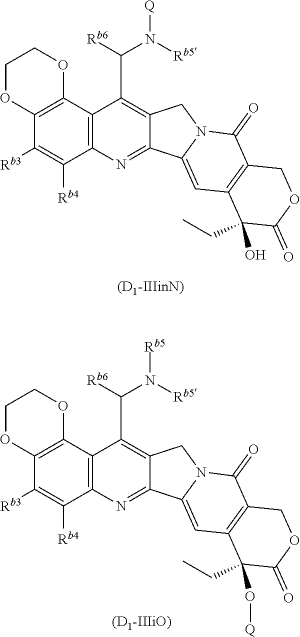

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁IIEN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-IV are represented by the formulae of:

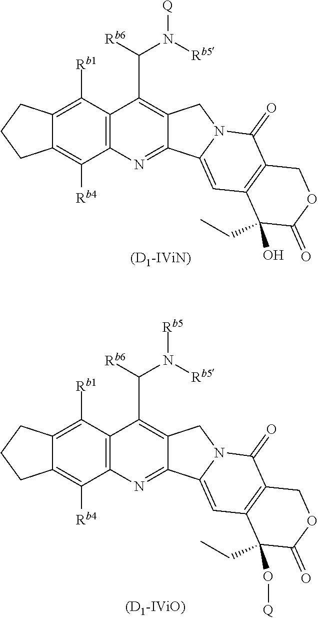

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁IViN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-V are represented by the formulae of:

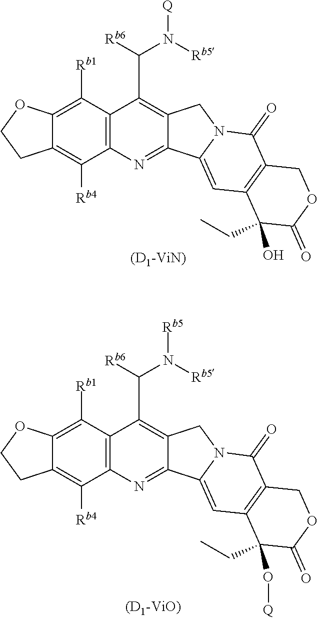

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D1ViN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-VI are represented by the formulae of:

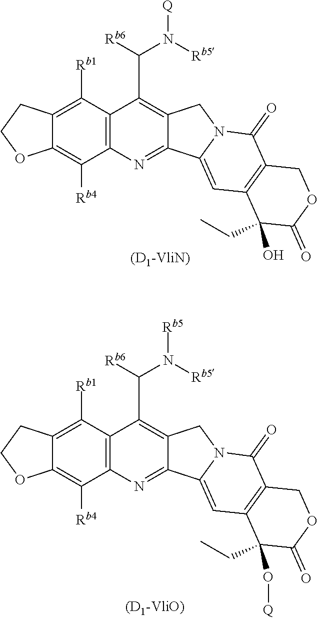

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁VIiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-VII are represented by the formulae of:

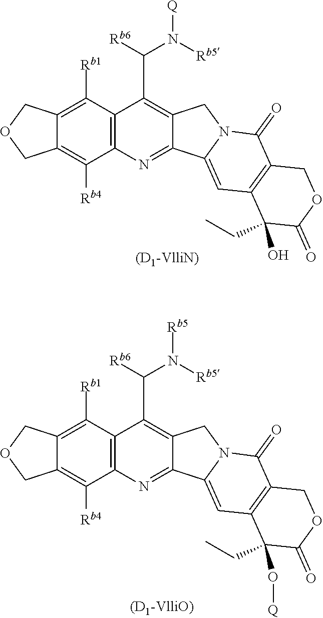

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁VIIiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-VIII are represented by the formulae of:

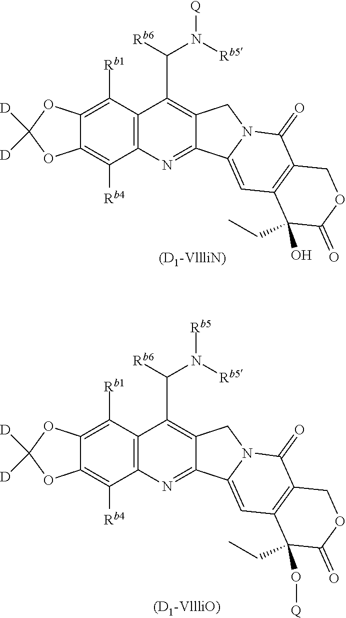

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁VIIIiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-IX are represented by the formulae of:

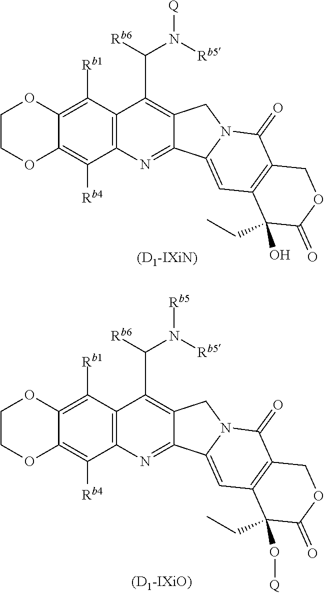

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁IXiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-X are represented by the formulae of:

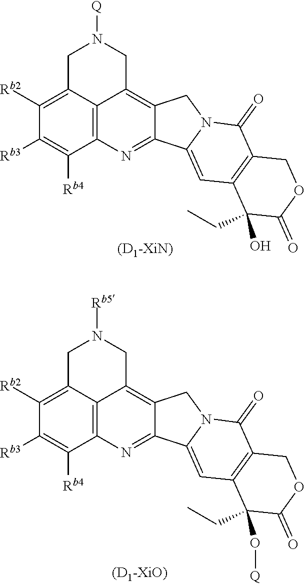

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI.

In another group of embodiments, the Camptothecin Conjugates comprised of a Drug Unit having formula D1-IIa are represented by the formulae of:

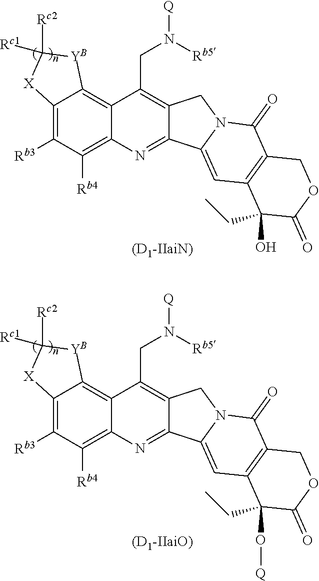

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁IIaiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-IIb are represented by the formulae of:

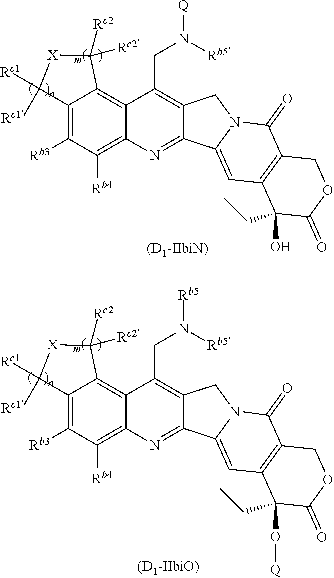

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-IIbiN wherein the nitrogen atom to which R^{b5}′ is bound is replaced by an oxygen atom and R^{b5}′ is absent, such that Q is attached to the Drug Unit via an oxygen atom of the Drug Unit.

In another group of embodiments, the Camptothecin Conjugates comprised of a Drug Unit having formula D1-IVa are represented by the formulae of:

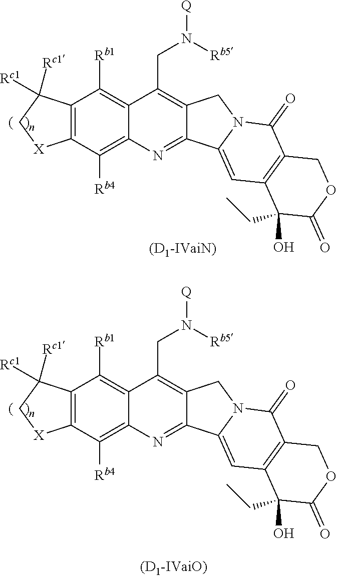

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-IVaiN wherein the nitrogen atom to which R^{x}′; is bound is replaced by an oxygen atom and R^{x}′; is absent, such that Q is attached to the Drug Unit via an oxygen atom of the Drug Unit.

In another group of embodiments, the Camptothecin Conjugates comprised of a Drug Unit having formula D1-IVb are represented by the formulae of:

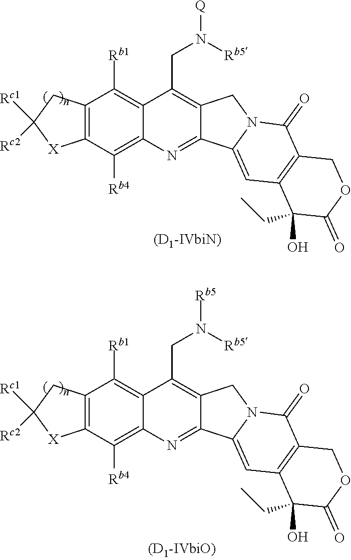

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-IVbiN wherein the nitrogen atom to which R^{x}′; is bound is replaced by an oxygen atom and R^{x}′; is absent, such that Q is attached to the Drug Unit via an oxygen atom of the Drug Unit.

In another group of embodiments, the Camptothecin Conjugates comprised of a Drug Unit having formula D1-Xa are represented by the formulae of:

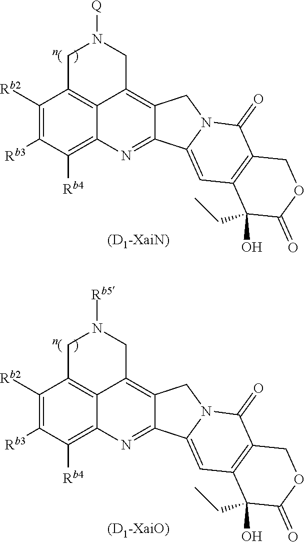

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI.

In another group of embodiments, the Camptothecin Conjugates comprised of a Drug Unit having formula D1-XI are represented by the formulae of:

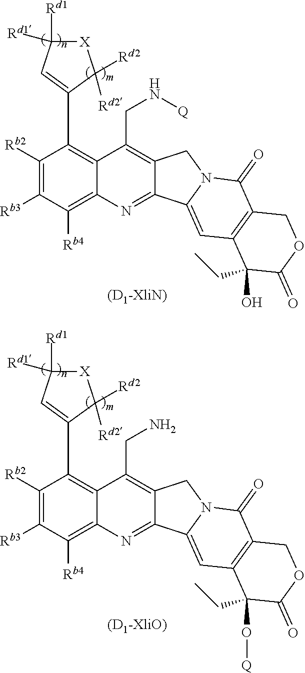

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-XIiN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-XII are represented by the formulae of:

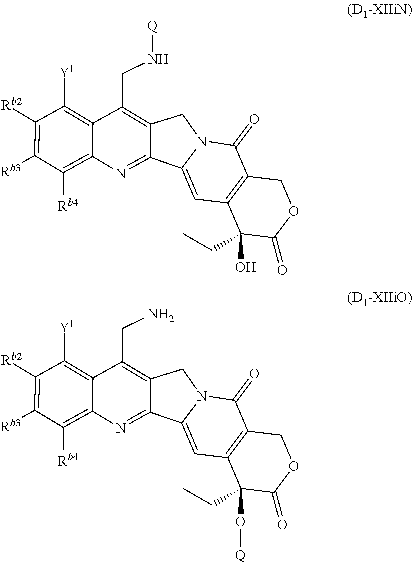

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-XIIiN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-XIII are represented by the formulae of:

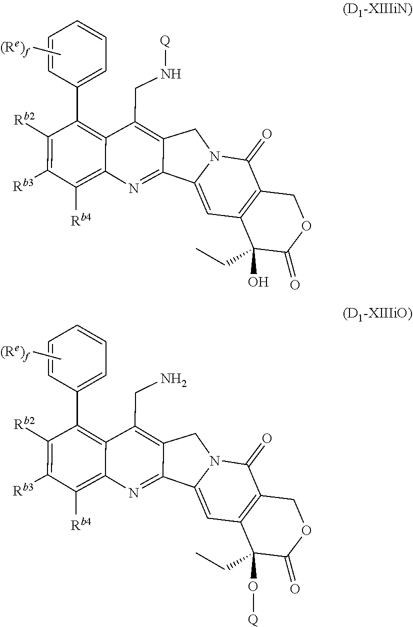

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-MIEN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-XIV are represented by the formulae of:

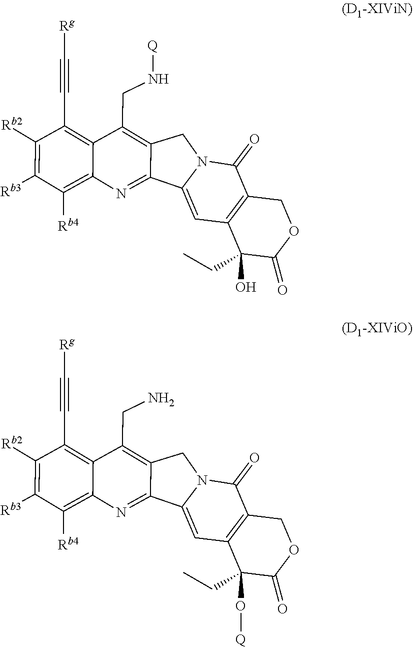

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-XIViN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-XV are represented by the formulae of:

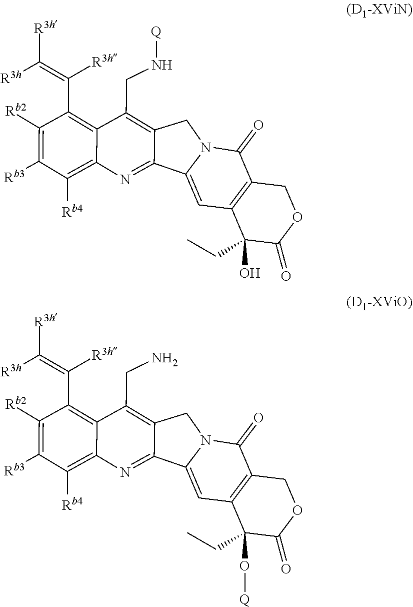

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-IIb, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-XViN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-XVI are represented by the formulae of:

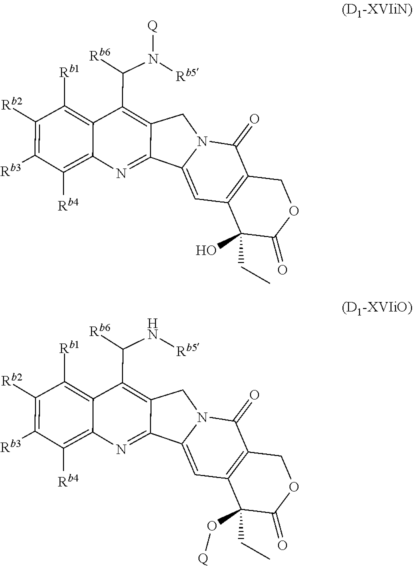

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-,-Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, and D1-XVI. Also provided herein are Camptothecin Conjugates corresponding to formula D₁-XVIiN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

In another group of embodiments, the Camptothecin Conjugates comprised of a Drug Unit having formula D1-CPT6 are represented by the formulae of:

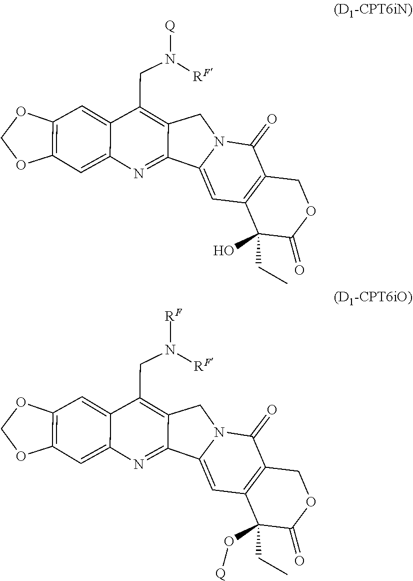

respectively, wherein Q has the formula of -Z-A-RL-, -Z-A-RL-Y-, -Z-A-S*-RL-, -Z-A-S*-RL-Y-, -Z-A-B(S*)-RL-, -Z-A-B(S*)-RL-Y-, -Z-A-, -Z-A-RL-, -Z-A-S*-W-, -Z-A-B(S*)-W-, -Z-A-S*-RL-, -Z-A-B(S*)-RL-, -Z-A-S*-W-RL-, or -Z-A-B(S*)-W-RL-; the groups L, Z, A, S*, B, RL, and Y have the meanings provided above and in any one of the embodiments specifically recited herein; and the remaining variables are as defined for D₁ D1-IIa, D1-Ib, D1-IVa, D1-IVb, D1-Xa, D1-XI, D1-XII, D1-XIII, D1-XIV, D1-XV, D1-XVI, and D1-CPT6. Also provided herein are Camptothecin Conjugates corresponding to formula D₁-CPT6iN wherein the nitrogen atom to which Q is bound is replaced by an oxygen atom and the hydrogen substituting the aforementioned nitrogen atom is absent, such that Q is attached to the Drug Unit via an oxygen atom of the Drug Unit.

Camptothecin molecules are known to undergo a pH dependent, reversible hydrolysis between a ring closed lactone form and a ring open carboxylate.

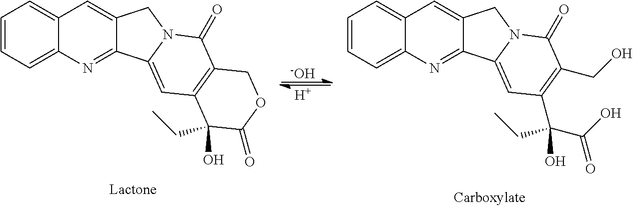

Without being bound by theory, it is believed that at acidic pH, the lactone form is favored while at physiologic pH the predominant form is the ring opened carboxylate. In biological systems camptothecin free drugs or drug-linkers, or conjugates thereof, may exist in either the lactone or carboxylate forms. Camptothecin-based antibody drug conjugates (ADC) have demonstrated activity to target cells regardless of the state of the lactone of the bound drug. Without being bound by theory, this effect is believed to be due to ADC processing in acidic intracellular vesicles, which favor equilibrium to the active closed-lactone form of camptothecin (Lau, U. Y. et al. Mol. Pharmaceutics 2018, 15, 9, 4063-4072).

It is to be understood that the Drug Units herein, as well as Drug-Linkers and conjugates thereof, can undergo equilibrium between the lactone and carboxylate forms. As such, for any of the lactone structures described herein, the carboxylate form is also to be understood to be within the scope of the present disclosure. All carboxylate forms of camptothecin structures depicted in the lactone form, including genericized formulae, are understood to be included herein in the same context as the lactone forms, as though each lactone structure was specifically and individually included in the carboxylate form.

### Camptothecin-Linker Compounds

In some embodiments, when preparing the Camptothecin Conjugates, it will be desirable to synthesize the full drug-linker combination prior to conjugation to a targeting agent. In such embodiments, Camptothecin-Linker Compounds as described herein, are intermediate compounds. In those embodiments, the Stretcher Unit in a Camptothecin-Linker compound is not yet covalently attached to the Ligand Unit (i.e., is a Stretcher Unit precursor, Z′), and therefore has a functional group for conjugation to a targeting ligand. In one embodiment, a Camptothecin-Linker compound is comprised of a Camptothecin compound (shown herein as formulae D₁ D₁ₐ, D_{1b}, or any subformula thereof,), and a Linker Unit (Q) comprising a Glycoside (e.g., Glucuronide) Unit as a Releasable Linker (RL) through which the Ligand Unit is connected to the Camptothecin.

In another embodiment, a Camptothecin-Linker Compound comprises a Camptothecin compound of formulae D₁ D₁ₐ, D_{1b}, or any subformula thereof, and a Linker Unit (Q) comprising a Releasable Linker (RL) that is other than a Glycoside (e.g., Glucuronide) Unit through which the Ligand Unit is connected to the conjugated Camptothecin compound. Thus, in either embodiment the Linker Unit comprises, in addition to RL, a Stretcher Unit precursor (Z′) comprising a functional group for conjugation to a targeting agent that is the precursor to the Ligand Unit and thus is capable of (directly or indirectly) connecting the RL to the Ligand Unit. In some of those embodiments a Parallel Connector Unit (B) when it is desired to add a Partitioning Agent (S*) as a side chain appendage. In any one of those embodiments, a Connector Unit (A) is present when it is desirable to add more distance between the Stretcher Unit and RL.

In one group of embodiments, a Camptothecin-Linker compound is comprised of a Camptothecin compound having formula D₁ D₁ₐ, D_{1b}, or any subformula thereof, and a Linker Unit (Q), wherein Q comprises a Releasable Linker (RL) that is a Glycoside (e.g., Glucuronide) Unit, directly attached to a Stretcher Unit precursor (Z′) or indirectly to Z′ through attachment to intervening component(s) of the Camptothecin-Linker compound's Linker Unit (i.e., A, S* and/or B(S*)), wherein Z′ is comprised of a functional group capable of forming a covalent bond to a targeting agent.

In another group of embodiments, a Camptothecin-Linker Compound is comprised of a Camptothecin having formula D₁ D₁ₐ, D_{1b}, or any subformula thereof, and a Linker Unit (Q), wherein Q comprises a Releasable Linker (RL) that is other than a Glycoside (e.g., Glucuronide) Unit (RL), directly attached to a Stretcher Unit precursor (Z′) or indirectly to Z′ through attachment to intervening component(s) of the Camptothecin-Linker Compound's Linker Unit (i.e., A, S* and/or B(S*)), wherein Z′ is comprised of a functional group capable of forming a covalent bond to a targeting agent.

In the context of the Camptothecin Conjugates and/or the Camptothecin-Linker Compounds—the assembly is best described in terms of its component groups. While some procedures are also described herein, the order of assembly and the general conditions to prepare the Conjugates and Compounds will be well understood by one of skill in the art.

In some embodiments, provided herein is a Camptothecin-Linker compound, wherein the compound is selected from the group consisting of the compounds in Table H. In some embodiments, provided herein is a Camptothecin Conjugate, wherein the Conjugate comprises a Ligand attached to a succinimide moeity or a succinic acid-amide moeity of a Drug-Linker moiety, wherein the Drug-Linker moeity comprises a compound of Table II, wherein the maleimide moeity is replaced by the succinimide or succinic acid-amide moiety.

TABLE II

| No. | Structure |
|---|---|
| 4 | 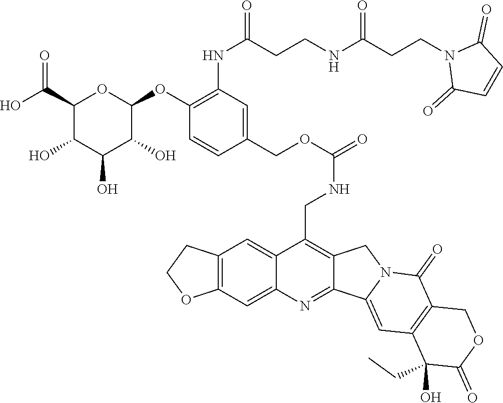 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCO3] |
| 4a | 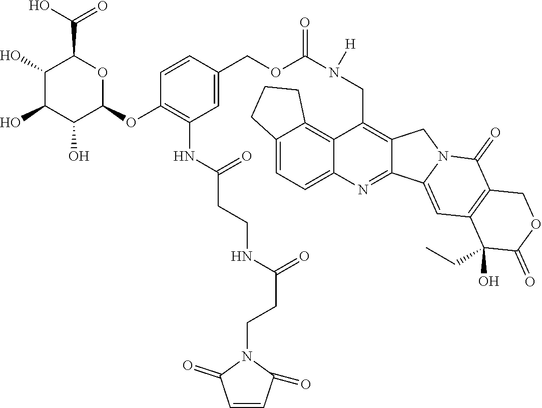 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCC3] |
| 4b | 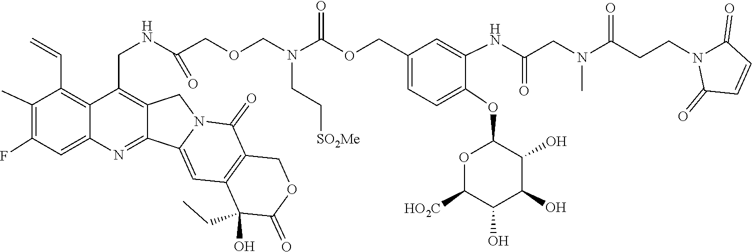 [SMILES: C=Cc1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4c | 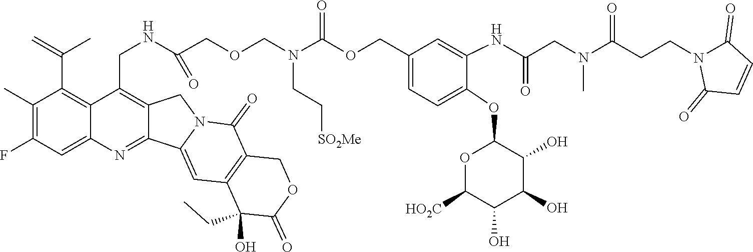 [SMILES: C=C(C)c1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4d | 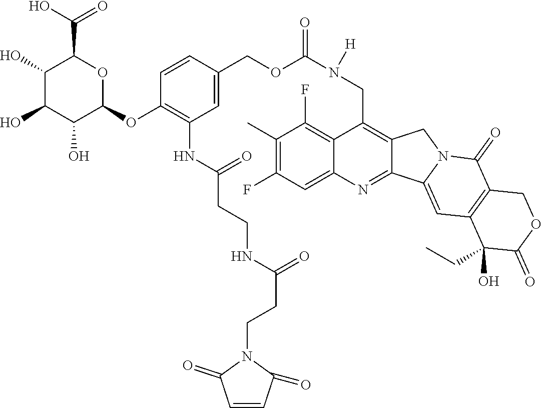 [SMILES: CC[C@@]1(O)C(=O)OCc2c1cc1n(c2=O)Cc2c-1nc1cc(F)c(C)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4e | 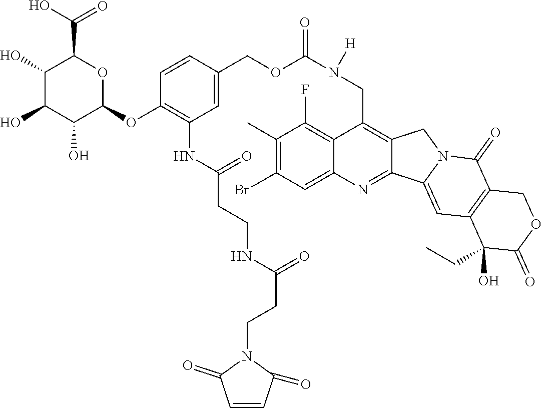 [SMILES: CC[C@@]1(O)C(=O)OCc2c1cc1n(c2=O)Cc2c-1nc1cc(Br)c(C)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4f | 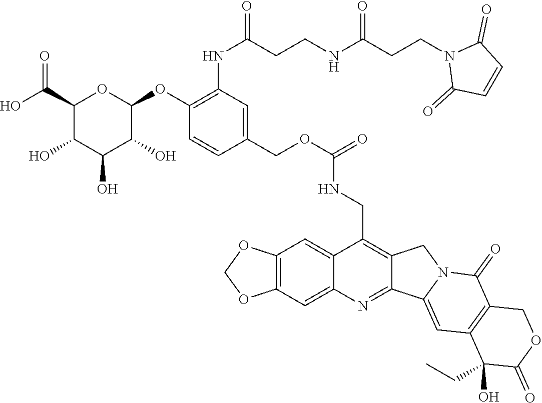 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCO3] |
| 4g | 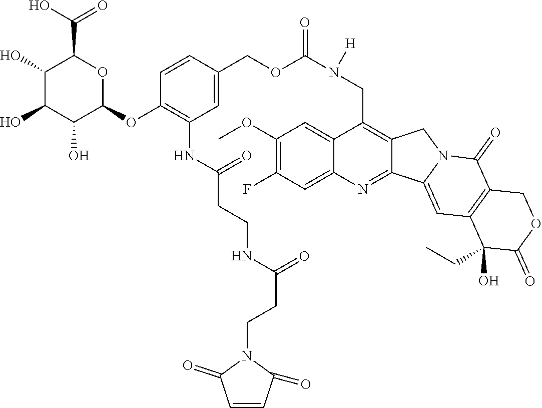 [SMILES: CC[C@@]1(O)C(=O)OCc2c1cc1n(c2=O)Cc2c-1nc1cc(F)c(OC)cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4h | 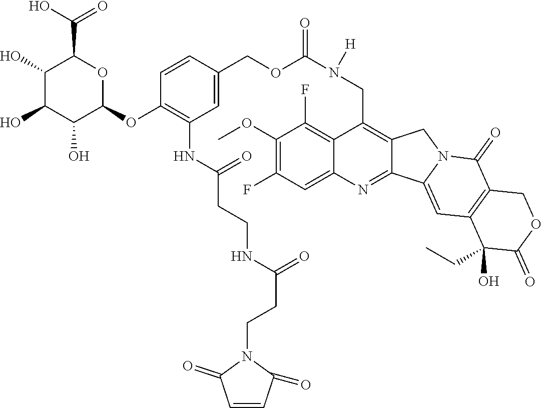 [SMILES: CC[C@@]1(O)C(=O)OCc2c1cc1n(c2=O)Cc2c-1nc1cc(F)c(OC)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4i | 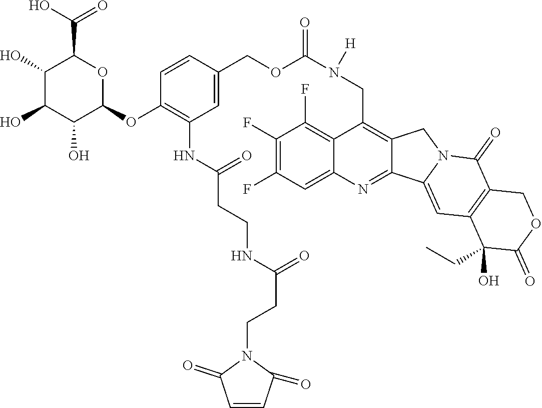 [SMILES: CC[C@@]1(O)C(=O)OCc2c1cc1n(c2=O)Cc2c-1nc1cc(F)c(F)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4j | 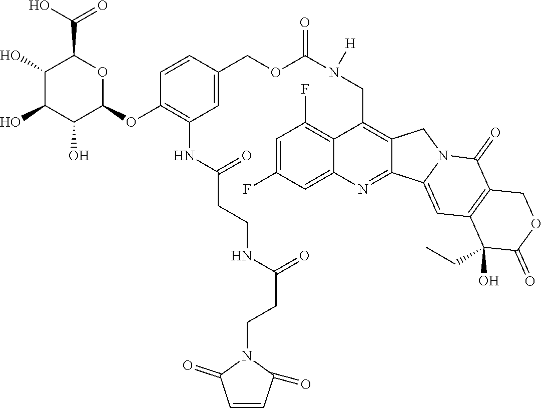 [SMILES: CC[C@@]1(O)C(=O)OCc2c1cc1n(c2=O)Cc2c-1nc1cc(F)cc(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4k | 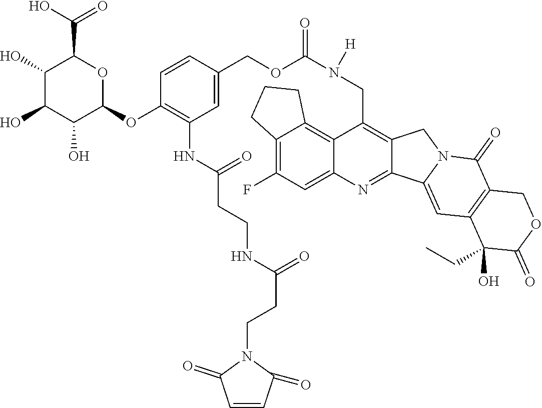 [SMILES: CC[C@@]1(O)C(=O)OCc2c1cc1n(c2=O)Cc2c-1nc1cc(F)c3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCC3] |
| 4l | 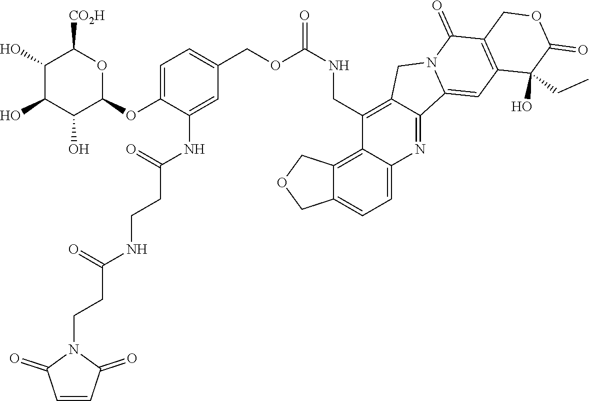 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)COC3] |
| 4m | 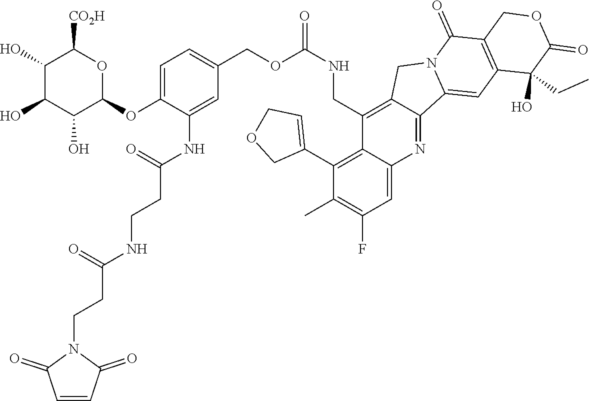 [SMILES: CC[C@@]1(O)C(=O)OCc2c1cc1n(c2=O)Cc2c-1nc1cc(F)c(C)c(C3=CCOC3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4n | 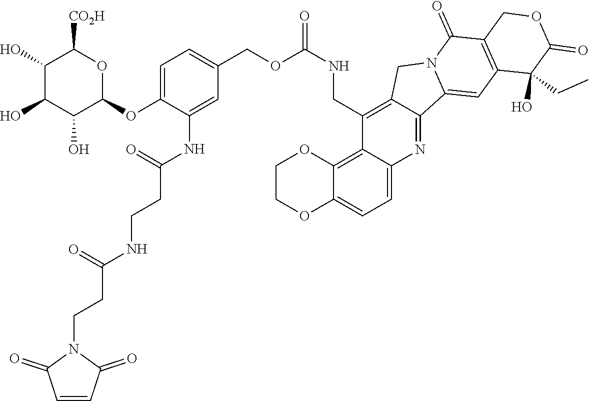 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCCO3] |
| 4o | 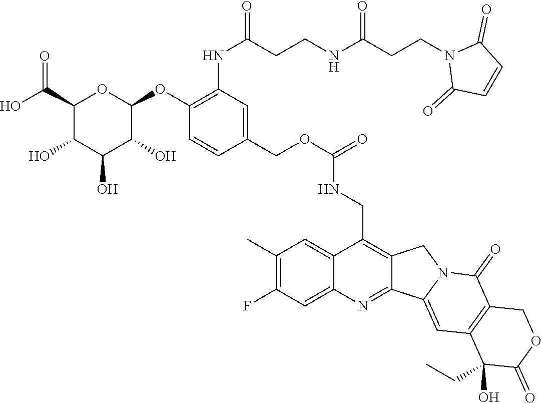 [SMILES: CC[C@@]1(O)C(=O)OCc2c1cc1n(c2=O)Cc2c-1nc1cc(F)c(C)cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4p | 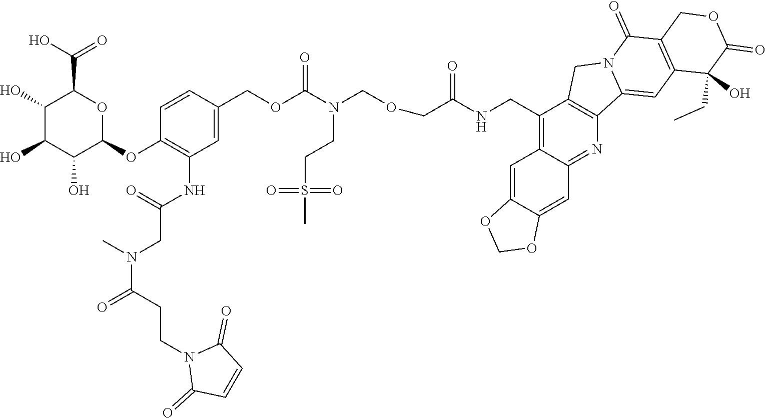 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)OCO3] |
| 4q | 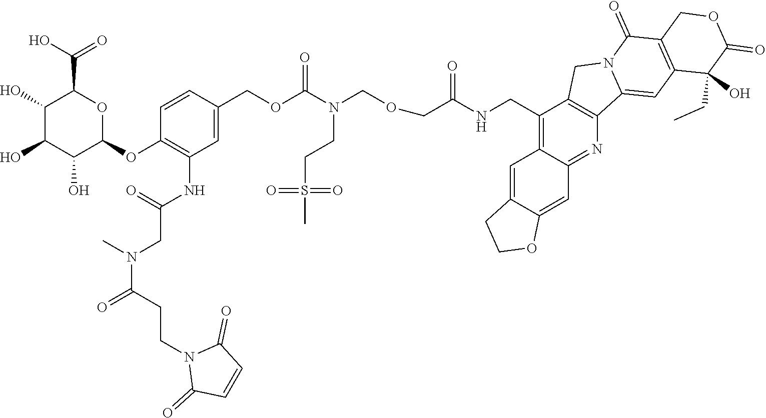 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)CCO3] |
| 4r | 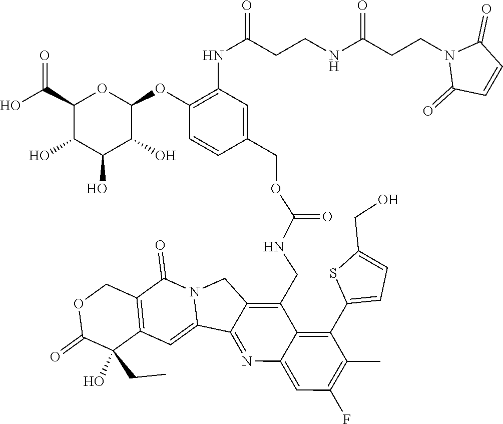 [SMILES: CC[C@@]1(O)C(=O)OCc2c1cc1n(c2=O)Cc2c-1nc1cc(F)c(C)c(-c3ccc(CO)s3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4s | 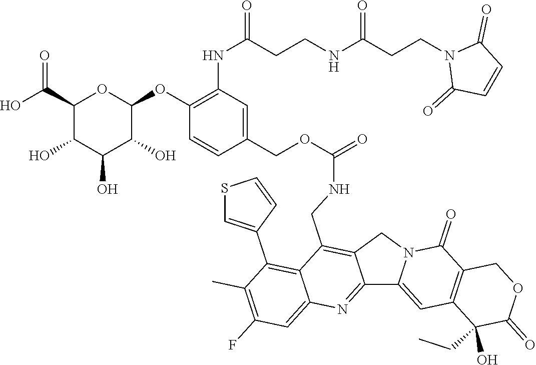 [SMILES: CC[C@@]1(O)C(=O)OCc2c1cc1n(c2=O)Cc2c-1nc1cc(F)c(C)c(-c3ccsc3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4t | 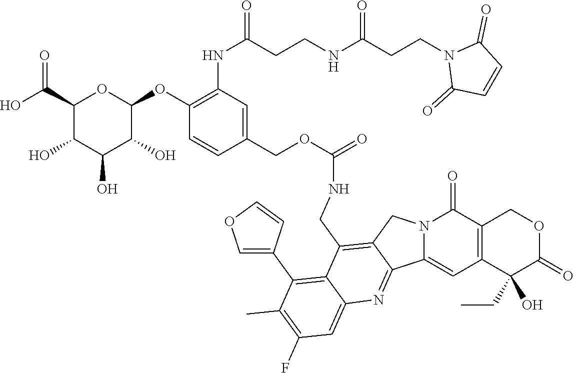 [SMILES: CC[C@@]1(O)C(=O)OCc2c1cc1n(c2=O)Cc2c-1nc1cc(F)c(C)c(-c3ccoc3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4u | 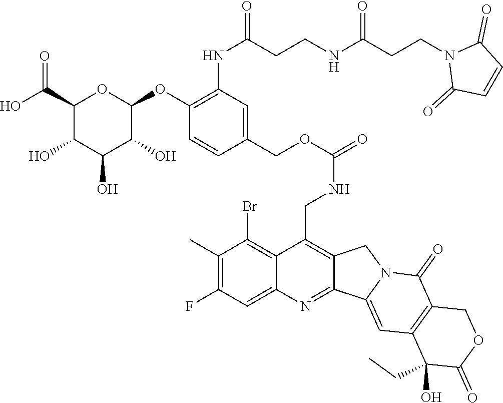 [SMILES: CC[C@@]1(O)C(=O)OCc2c1cc1n(c2=O)Cc2c-1nc1cc(F)c(C)c(Br)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4v | 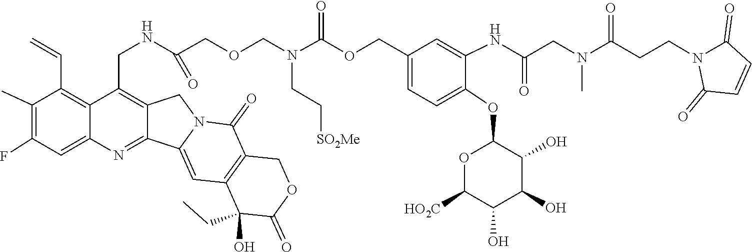 [SMILES: C=Cc1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4w | 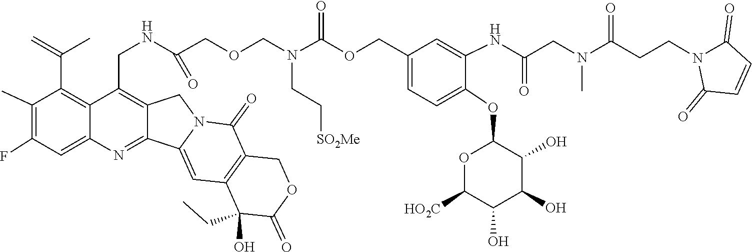 [SMILES: C=C(C)c1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4x | 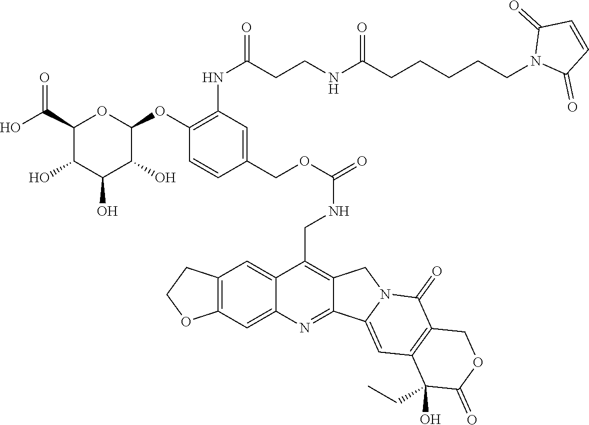 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1)CCO3] |
| 4y | 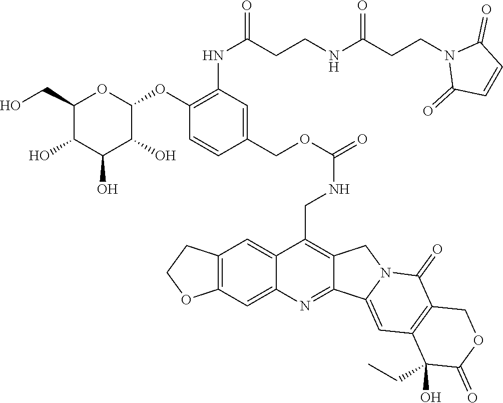 [SMILES: CC[C@@]1(O)C(=O)OCc2c1cc1n(c2=O)Cc2c-1nc1cc3c(cc1c2CNC(=O)OCc1ccc(O[C@H]2O[C@H](CO)[C@@H](O)[C@H](O)[C@H]2O)c(NC(=O)CCNC(=O)CCN2C(=O)C=CC2=O)c1)CCO3] |
| 4z | 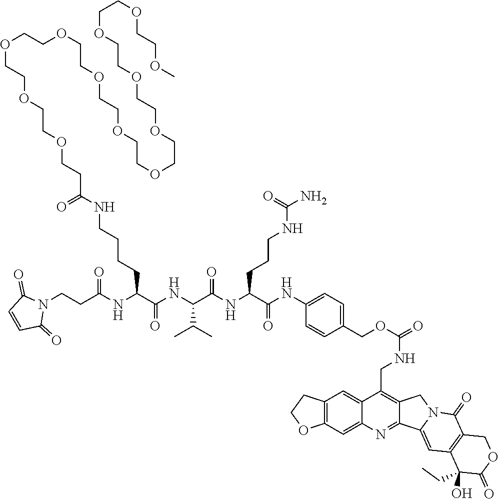 [SMILES: CC[C@@]1(O)C(=O)OCc2c1cc1n(c2=O)Cc2c-1nc1cc3c(cc1c2CNC(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOC)NC(=O)CCN2C(=O)C=CC2=O)C(C)C)cc1)CCO3] |
| 4aa | 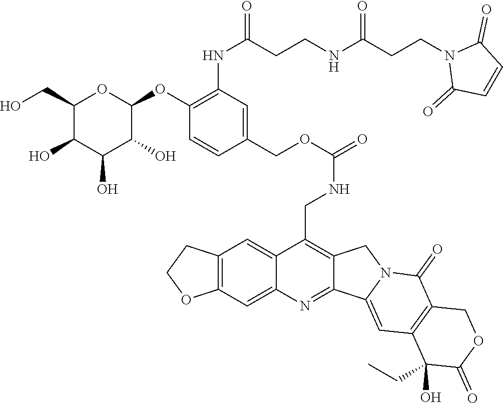 [SMILES: CC[C@@]1(O)C(=O)OCc2c1cc1n(c2=O)Cc2c-1nc1cc3c(cc1c2CNC(=O)OCc1ccc(O[C@@H]2O[C@H](CO)[C@H](O)[C@H](O)[C@H]2O)c(NC(=O)CCNC(=O)CCN2C(=O)C=CC2=O)c1)CCO3] |
| 4ab | 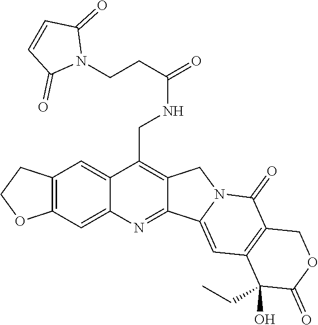 [SMILES: CC[C@@]1(O)C(=O)OCc2c1cc1n(c2=O)Cc2c-1nc1cc3c(cc1c2CNC(=O)CCN1C(=O)C=CC1=O)CCO3] |
| 4ac | 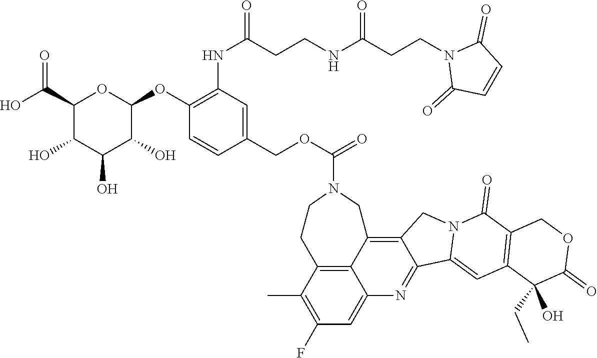 [SMILES: CC[C@@]1(O)C(=O)OCc2c1cc1n(c2=O)Cc2c-1nc1cc(F)c(C)c3c1c2CN(C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CC3] |
| 4ad | 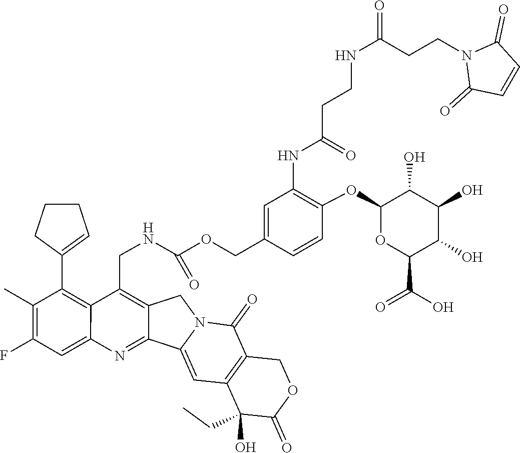 [SMILES: CC[C@@]1(O)C(=O)OCc2c1cc1n(c2=O)Cc2c-1nc1cc(F)c(C)c(C3=CCCC3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4ae | 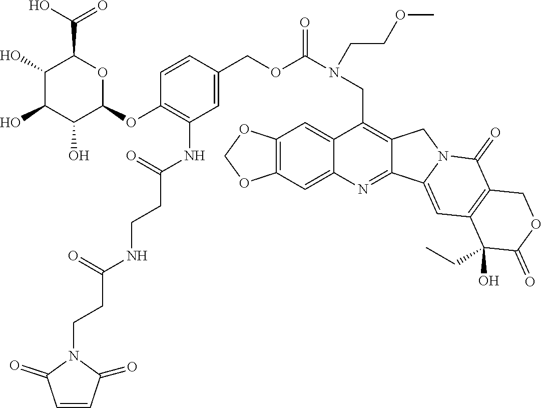 [SMILES: CC[C@@]1(O)C(=O)OCc2c1cc1n(c2=O)Cc2c-1nc1cc3c(cc1c2CN(CCOC)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCO3] |

### Component Groups

### Ligand Units:

In some embodiments of the invention, a Ligand Unit is present. The Ligand Unit (L-) is a targeting agent that specifically binds to a target moiety. In one group of embodiments, the Ligand Unit specifically and selectively binds to a cell component (a Cell Binding Agent) or to another target molecule of interest. The Ligand Unit acts to target and present the camptothecin (such as one of formula D₁ D₁ₐ, D_{1b}, or any subformula thereof) to the particular target cell population with which the Ligand Unit interacts due to the presence of its targeted component or molecule and allows for subsequent release of free drug within (i.e., intracellularly) or within the vicinity of the target cells (i.e., extracellularly). Ligand Units, L, include, but are not limited to, proteins, polypeptides, and peptides. Suitable Ligand Units include, for example, antibodies, e.g., full-length antibodies and antigen binding fragments thereof, interferons, lymphokines, hormones, growth factors, colony-stimulating factors, vitamins, nutrient-transport molecules (such as, but not limited to, transfenrin), or any other cell binding molecule or substance. In some embodiments, the Ligand Unit (L) is from an antibody or a non-antibody protein targeting agent.

In one group of embodiments a Ligand Unit is bonded to Q (a Linker Unit) which comprises a Glucuronide Releasable Linker. As noted above, still other linking components can be present in the conjugates described herein to serve the purpose of providing additional space between the Camptothecin drug compound and the Ligand Unit (e.g., a Stretcher Unit and optionally a Connector Unit, A), or providing attributes to the composition to increases solubility (e.g., a Partitioning Agent, S*). In some of those embodiments, the Ligand Unit is bonded to Z of the Linker Unit via a heteroatom of the Ligand Unit. Heteroatoms that may be present on a Ligand Unit for that bonding include sulfur (in one embodiment, from a sulfhydryl group of a targeting ligand), oxygen (in one embodiment, from a carboxyl or hydroxyl group of a targeting ligand) and nitrogen, optionally substituted (in one embodiment, from a primary or secondary amine functional group of a targeting ligand or in another embodiment from an optionally substituted amide nitrogen). Those heteroatoms can be present on the targeting ligand in the ligand's natural state, for example in a naturally occurring antibody, or can be introduced into the targeting ligand via chemical modification or biological engineering.

In one embodiment, a targeting agent that is a precursor to a Ligand Unit has a sulfhydryl functional group so that the Ligand Unit is bonded to the Linker Unit via the sulfur atom of the sulfhydryl functional group.

In another embodiment, a targeting agent that is a precursor to Ligand Unit has one or more lysine residues that are capable of reacting with activated esters (such esters include, but are not limited to, N-hydroxysuccimide, pentafluorophenyl, and p-nitrophenyl esters) of a Stretcher Unit precursor of a Camptothecin-Linker Compound intermediate and thus provides an amide bond consisting of the nitrogen atom of the Ligand Unit and the C═O group of the Linker Unit's Stretcher Unit.

In yet another aspect, a targeting agent that is a precursor to Ligand Unit has one or more lysine residues capable of chemical modification to introduce one or more sulfhydryl groups. In those embodiments, the Ligand Unit is covalently attached to the Linker Unit via the sulfhydryl functional group's sulfur atom. The reagents that can be used to modify lysines in that manner include, but are not limited to, N-succinimidyl S-acetylthioacetate (SATA) and 2-Iminothiolane hydrochloride (Traut's Reagent).

In another embodiment, a targeting agent that is a precursor to a Ligand Unit has one or more carbohydrate groups capable of modification to provide one or more sulfhydryl functional groups. The chemically modified Ligand Unit in a Camptothecin Conjugate is bonded to a Linker Unit component (e.g., a Stretcher Unit) via the sulfur atom of the sulfhydryl functional group.

In yet another embodiment, a targeting agent that is a precursor to a Ligand Unit has one or more carbohydrate groups that can be oxidized to provide an aldehyde (—CHO) functional group (see, e.g., Laguzza, et al., 1989, J. Med. Chem. 32(3):548-55). In these embodiments, the corresponding aldehyde interacts with a reactive site on a Stretcher Unit precursor to form a bond between the Stretcher Unit and the Ligand Unit. Reactive sites on a Stretcher Unit precursor that capable of interacting with a reactive carbonyl-containing functional group on a targeting Ligand Unit include, but are not limited to, hydrazine and hydroxylamine. Other protocols for the modification of proteins for the attachment of Linker Units (Q) or related species are described in Coligan et al., Current Protocols in Protein Science, vol. 2, John Wiley & Sons (2002) (incorporated herein by reference).

In some aspects, a targeting agent that is a precursor to a Ligand Unit t is capable of forming a bond by interacting with a reactive functional group on a Stretcher Unit precursor (Z′) to form a covalent bond between the Stretcher Unit (Z) and the Ligand Unit, which corresponds in structure to the targeting agent. The functional group of Z′ having that capability for interacting with a targeting agent will depend on the nature of the targeting agent that will correspond in structure to the Ligand Unit. In some embodiments, the reactive group is a maleimide that is present on a Stretcher Unit prior to its attachment to form a Ligand Unit (i.e., a maleimide moiety of a Stretcher Unit precursor). Covalent attachment of a Ligand Unit to a Stretcher Unit is accomplished through a sulfhydryl functional group of a targeting agent that is a precursor to a Ligand Unit interacting with the maleimide functional group of Z′ to form a thio-substituted succinimide. The sulfhydryl functional group can be present on the targeting agent in the targeting agent's natural state, for example, in a naturally occurring residue, or can be introduced into the targeting agent via chemical modification or by biological engineering.

In still another embodiment, the Ligand Unit is from an antibody and the sulfhydryl group is generated by reduction of an interchain disulfide of the antibody. Accordingly, in some embodiments, the Linker Unit is conjugated to a cysteine residue from reduced interchain disulfide(s).

In yet another embodiment, the Ligand Unit is from an antibody and the sulfhydryl functional group is chemically introduced into the antibody, for example, by introduction of a cysteine residue. Accordingly, in some embodiments, the Linker Unit (with or without an attached Camptothecin) is conjugated to a Ligand Unit through an introduced cysteine residue of a Ligand Unit.

It has been observed for bioconjugates that the site of drug conjugation can affect a number of parameters including ease of conjugation, drug-linker stability, effects on biophysical properties of the resulting bioconjugates, and in vitro cytotoxicity. With respect to drug-linker stability, the site of conjugation of a drug-linker moiety to a Ligand Unit can affect the ability of the conjugated drug-linker moiety to undergo an elimination reaction, in some instances, to cause premature release of free drug. Sites for conjugation on a targeting agent include, for example, a reduced interchain disulfide as well as selected cysteine residues at engineered sites. In some embodiments conjugation methods to form Camptothecin Conjugates as described herein use thiol residues at genetically engineered sites that are less susceptible to the elimination reaction (e.g., positions 239 according to the EU index as set forth in Kabat) in comparison to conjugation methods that use thiol residues from a reduced disulfide bond. In other embodiments conjugation methods to form Camptothecin Conjugates as described herein use thiol residues resulting from interchain disulfide bond reduction.

In some embodiments, a Camptothecin Conjugate comprises a non-immunoreactive protein, polypeptide, or peptide as its Ligand Unit. Accordingly, in some embodiments, the Ligand Unit is from a non-immunoreactive protein, polypeptide, or peptide. Examples include, but are not limited to, transferrin, epidermal growth factors (“EGF”), bombesin, gastrin, gastrin-releasing peptide, platelet-derived growth factor, IL-2, IL-6, transforming growth factors (“TGF”), such as TGF-α and TGF-β, vaccinia growth factor (“VGF”), insulin and insulin-like growth factors I and II, somatostatin, lectins and apoprotein from low density lipoprotein.

Particularly preferred Ligand Units are from antibodies. Accordingly, in any one of the embodiments described herein, the Ligand Unit is from an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest in some embodiments is prepared by using any technique known in the art, which provides for production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies can be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

An antibody useful for practicing the invention is an intact antibody or a functionally active fragment, derivative or analog of an antibody, wherein the antibody or fragment thereof is capable of immunospecific binding to target cells (e.g., cancer cell antigens, viral antigens, or microbial antigens) or other antibodies that are bound to tumor cells or matrix. In this regard, “functionally active” means that the fragment, derivative or analog is able to immunospecifically bind to target cells. To determine which CDR sequences bind the antigen, synthetic peptides containing the CDR sequences in some embodiments are used in binding assays with the antigen by a binding assay method known in the art (e.g., the Biacore™ assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Other useful antibodies include fragments of antibodies such as, but not limited to, F(ab′)2 fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which in some embodiments are made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies in some embodiments are produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; Berter et al., Science (1988) 240: 1041-1043; Liu et al., Proc. Natl. Acad. Sci. USA (1987) 84: 3439-3443; Liu et al., J. Immunol. (1987) 139: 3521-3526; Sun et al., Proc. Natl. Acad. Sci. USA (1987) 84: 214-218; Nishimura et al., Cancer. Res. (1987) 47: 999-1005; Wood et al., Nature (1985) 314: 446 449; Shaw et al., J. Natl. Cancer Inst. (1988) 80: 1553-1559; Morrison, Science (1985) 229: 1202-1207; Oi et al., BioTechniques (1986) 4: 214-221; U.S. Pat. No. 5,225,539; Jones et al., Nature (1986) 321: 552-525; Verhoeyan et al., Science (1988) 239: 1534-1536; and Beidler et al., J. Immunol. (1988) 141: 4053-4060; each of which is incorporated herein by reference in its entirety.

Completely human antibodies in some instances (e.g., when immunogenicity to a non-human or chimeric antibody may occur) are more desirable and in some embodiments are produced using transgenic mice that are incapable of expressing endogenous immunoglobulin heavy and light chains genes, but which are capable of expressing human heavy and light chain genes.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivitization by known protecting/blocking groups, proteolytic cleavage, linkage to a cellular antibody unit or other protein, etc. In some embodiments one or more of those numerous chemical modifications are carried out by known techniques including, but not limited to, specific chemical cleavage, acetylation, formylation, metabolic synthesis in the presence of tunicamycin, etc. In other embodiments, an analog or derivative of an antibody contains one or more unnatural amino acids, which is sometimes in combination with one or more of the above-described chemical modifications.

In some embodiments the antibody has one or more modifications (e.g., substitutions, deletions or additions) in amino acid residues that interact with Fc receptors. Those include modifications in amino acid residues identified as involved in the interaction between the anti-Fc domain and the FcRn receptor (see, e.g., International Publication No. WO 97/34631, which is incorporated herein by reference in its entirety).

In some embodiments, antibodies immunospecific for a cancer cell antigen are obtained commercially or produced by a method known to one of skill in the art such as, recombinant expression techniques. The nucleotide sequence encoding antibodies immunospecific for a cancer cell antigen is sometimes obtained, e.g., from the GenBank database or a database like it, the literature publications, or by routine cloning and sequencing.

In a specific embodiment, a known antibody for the treatment of cancer is used.

In another specific embodiment, an antibody for the treatment of an autoimmune disease is used in accordance with the compositions and methods of the invention.

In certain embodiments, useful antibodies bind to a receptor or a receptor complex expressed on an activated lymphocyte. That receptor or receptor complex, in some embodiments, is an immunoglobulin gene superfamily member, a TNF receptor superfamily member, an integrin, a cytokine receptor, a chemokine receptor, a major histocompatibility protein, a lectin, or a complement control protein.

In some embodiments, the antibody that is incorporated into a Camptothecin Conjugate will specifically bind CD19, CD30, CD33, CD70 or LIV-1.

Exemplary antigens are provided below. Exemplary antibodies that bind the indicated antigen are shown in parentheses.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD 147 (exemplary antibodies include gavilimomab and metuzumab), CD19, CD20 (exemplary antibodies include divozilimab and ibritumomab tiuxetan), CD274 also known as PD-L 1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SIT1, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD 13A, Mincle, NOX 1, SLC 10A2, SLC 12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD 112, CD 155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAM1, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN 1, Gp 100, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS 1 R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, 0Y-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD 115 (exemplary antibodies include axatilimab, cabiralizumab, and emactuzumab), CD 123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAXS, and WT 1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, 1TGAV (exemplary antibodies include abituzumab), ITGB6, and 1TGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM 12, ADAM9, TMPRSS 11 D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAILI, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD 163, CD 19, CD20 (exemplary antibodies include rituximab, ocrelizumab, divozilimab; ibritumomab tiuxetan), CD25 (exemplary antibodies include basiliximab), CD274 also known as PD-L 1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD 112, CD 155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL 1 RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD 115 (exemplary antibodies include axatilimab, cabiralizumab, and emactuzumab), CD 123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MefTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR 1 (exemplary antibodies include faralimomab), and IFNAR2.

In some embodiments, the antigen is CD30. In some embodiments, the antibody is an antibody or antigen-binding fragment that binds to CD30, such as described in International Patent Publication No. WO 02/43661. In some embodiments, the anti-CD30 antibody is cAC 10, which is described in International Patent Publication No. WO 02/43661. cAC 10 is also known as brentuximab. In some embodiments, the anti-CD30 antibody comprises the CDRs of cAC 10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some embodiments, the antigen is CD70. In some embodiments, the antibody is an antibody or antigen-binding fragment that binds to CD70, such as described in International Patent Publication No. WO 2006/113909. In some embodiments, the antibody is a h 1 F6 anti-CD70 antibody, which is described in International Patent Publication No. WO 2006/113909. h 1 F6 is also known as vorsetuzumab. In some embodiments, the anti-CD70 antibody comprises a heavy chain variable region comprising the three CDRs of SEQ ID NO:12 and a light chain variable region comprising the three CDRs of SEQ ID NO:13. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD70 antibody comprises a heavy chain variable region comprising an amino acid sequence that is at least 95%, at least 96%, at least 97%, at last 98%, at least 99%, or 100% identical to the amino acid sequence of SEQ ID NO: 12 and a light chain variable region comprising an amino acid sequence that is at least 95% at least 96%, at least 97%, at last 98%, at least 99%, or 100% identical to the amino acid sequence of SEQ ID NO: 13. In some embodiments, the anti-CD30 antibody comprises a heavy chain comprising the amino acid sequence of SEQ ID NO: 14 and a light chain comprising the amino acid sequence of SEQ ID NO: 15.

In some embodiments, the antigen is interleukin-1 receptor accessory protein (IL1RAP). IL1RAP is a co-receptor of the IL 1 receptor (IL 1 R 1) and is required for interleukin-1 (IL 1) signaling. IL 1 has been implicated in the resistance to certain chemotherapy regimens. IL1RAP is overexpressed in various solid tumors, both on cancer cells and in the tumor microenvironment, but has low expression on normal cells. IL1RAP is also overexpressed in hematopoietic stem and progenitor cells, making it a candidate to target for chronic myeloid leukemia (CML). IL1RAP has also been shown to be overexpressed in acute myeloid leukemia (AML). Antibody binding to IL1RAP could block signal transduction from IL-1 and IL-33 into cells and allow NK-cells to recognize tumor cells and subsequent killing by antibody dependent cellular cytotoxicity (ADCC).

In some embodiments, the antigen is ASCT2. ASCT2 is also known as SLC 1 A5. ASCT2 is a ubiquitously expressed, broad-specificity, sodium-dependent neutral amino acid exchanger. ASCT2 is involved in glutamine transport. ASCT2 is overexpressed in different cancers and is closely related to poor prognosis. Downregulating ASCT2 has been shown to suppress intracellular glutamine levels and downstream glutamine metabolism, including glutathione production. Due to its high expression in many cancers, ASCT2 is a potential therapeutic target. These effects attenuated growth and proliferation, increased apoptosis and autophagy, and increased oxidative stress and mTORC 1 pathway suppression in head and neck squamous cell carcinoma (HNSCC). Additionally, silencing ASCT2 improved the response to cetuximab in HNSCC.

In some embodiments, an antibody-drug conjugate provided herein binds to TROP2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 16, 17, 18, 19, 20, and 21, respectively. In some embodiments, the antibody of the antibody drug conjugate comprises a heavy chain variable region comprising the amino acid sequence of SEQ ID NO: 22 and a light chain variable region comprising the amino acid sequence of SEQ ID NO: 23. In some embodiments, the antibody of the antibody drug conjugate is sacituzumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 24, 25, 26, 27, 28, and 29, respectively. In some embodiments, the antibody of the antibody drug conjugate comprises a heavy chain variable region comprising the amino acid sequence of SEQ ID NO: 30 and a light chain variable region comprising the amino acid sequence of SEQ ID NO: 31. In some embodiments, the antibody of the antibody drug conjugate is datopotamab.

In some embodiments, an antibody-drug conjugate provided herein binds to MICA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 32, 33, 34, 35, 36, and 37, respectively. In some embodiments, the antibody of the antibody drug conjugate comprises a heavy chain variable region comprising the amino acid sequence of SEQ ID NO: 38 and a light chain variable region comprising the amino acid sequence of SEQ ID NO: 39. In some embodiments, the antibody of the antibody drug conjugate is h1 D5v 11 hIgG 1 K. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 40, 41, 42, 43, 44, and 45, respectively. In some embodiments, the antibody of the antibody drug conjugate comprises a heavy chain variable region comprising the amino acid sequence of SEQ ID NO: 46 and a light chain variable region comprising the amino acid sequence of SEQ ID NO: 47. In some embodiments, the antibody of the antibody drug conjugate is MICA.36 hIgG 1 K G236A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 48, 49, 50, 51, 52, and 53, respectively. In some embodiments, the antibody of the antibody drug conjugate comprises a heavy chain variable region comprising the amino acid sequence of SEQ ID NO: 54 and a light chain variable region comprising the amino acid sequence of SEQ ID NO: 55. In some embodiments, the antibody of the antibody drug conjugate is h3F9 H1 L3 hIgG 1 K. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 56, 57, 58, 59, 60, and 61, respectively. In some embodiments, the antibody of the antibody drug conjugate comprises a heavy chain variable region comprising the amino acid sequence of SEQ ID NO: 62 and a light chain variable region comprising the amino acid sequence of SEQ ID NO: 63. In some embodiments, the antibody of the antibody drug conjugate is CM33322 Ab28 hIgG 1 K.

In some embodiments, an antibody-drug conjugate provided herein binds to CD24. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 64, 65, 66, 67, 68, and 69, respectively. In some embodiments, the antibody of the antibody drug conjugate comprises a heavy chain variable region comprising the amino acid sequence of SEQ ID NO: 70 and a light chain variable region comprising the amino acid sequence of SEQ ID NO: 71. In some embodiments, the antibody of the antibody drug conjugate is SWA11.

In some embodiments, an antibody-drug conjugate provided herein binds to TTGay. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 72, 73, 74, 75, 76, and 77, respectively. In some embodiments, the antibody of the antibody drug conjugate comprises a heavy chain variable region comprising the amino acid sequence of SEQ ID NO: 78 and a light chain variable region comprising the amino acid sequence of SEQ ID NO: 79. In some embodiments, the antibody of the antibody drug conjugate is intetumumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 80, 81, 82, 83, 84, and 85, respectively. In some embodiments, the antibody of the antibody drug conjugate comprises a heavy chain variable region comprising the amino acid sequence of SEQ ID NO: 86 and a light chain variable region comprising the amino acid sequence of SEQ ID NO: 87. In some embodiments, the antibody of the antibody drug conjugate is abituzumab.

In some embodiments, an antibody-drug conjugate provided herein binds to gpA33. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 88, 89, 90, 91, 92, and 93, respectively. In some embodiments, the antibody of the antibody drug conjugate comprises a heavy chain variable region comprising the amino acid sequence of SEQ ID NO: 94 and a light chain variable region comprising the amino acid sequence of SEQ ID NO: 95.

In some embodiments, an antibody-drug conjugate provided herein binds to IL1Rap. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 96, 97, 98, 99, 100, and 101, respectively. In some embodiments, the antibody of the antibody drug conjugate comprises a heavy chain variable region comprising the amino acid sequence of SEQ ID NO: 102 and a light chain variable region comprising the amino acid sequence of SEQ ID NO: 103. In some embodiments, the antibody of the antibody drug conjugate is nidanilimab.

In some embodiments, an antibody-drug conjugate provided herein binds to EpCAM. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 104, 105, 106, 107, 108, and 109, respectively. In some embodiments, the antibody of the antibody drug conjugate comprises a heavy chain variable region comprising the amino acid sequence of SEQ ID NO: 110 and a light chain variable region comprising the amino acid sequence of SEQ ID NO: 111. In some embodiments, the antibody of the antibody drug conjugate is adecatumumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 112, 113, 114, 115, 116, and 117, respectively. In some embodiments, the antibody of the antibody drug conjugate comprises a heavy chain variable region comprising the amino acid sequence of SEQ ID NO: 118 and a light chain variable region comprising the amino acid sequence of SEQ ID NO: 119. In some embodiments, the antibody of the antibody drug conjugate comprises a heavy chain variable region comprising the amino acid sequence of SEQ ID NO: 118 and a light chain variable region comprising the amino acid sequence of SEQ ID NO: 1182. In some embodiments, the antibody of the antibody drug conjugate is Ep157305. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 120, 121, 122, 123, 124, and 125, respectively. In some embodiments, the antibody of the antibody drug conjugate comprises a heavy chain variable region comprising the amino acid sequence of SEQ ID NO: 126 and a light chain variable region comprising the amino acid sequence of SEQ ID NO: 127. In some embodiments, the antibody of the antibody drug conjugate is Ep3-171. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 128, 129, 130, 131, 132, and 133, respectively. In some embodiments, the antibody of the antibody drug conjugate comprises a heavy chain variable region comprising the amino acid sequence of SEQ ID NO: 134 and a light chain variable region comprising the amino acid sequence of SEQ ID NO: 135. In some embodiments, the antibody of the antibody drug conjugate is Ep3622w94. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 136, 137, 138, 139, 140, and 141, respectively. In some embodiments, the antibody of the antibody drug conjugate comprises a heavy chain variable region comprising the amino acid sequence of SEQ ID NO: 142 and a light chain variable region comprising the amino acid sequence of SEQ ID NO: 143. In some embodiments, the antibody of the antibody drug conjugate is EpING1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 144, 145, 146, 147, 148, and 149, respectively. In some embodiments, the antibody of the antibody drug conjugate comprises a heavy chain variable region comprising the amino acid sequence of SEQ ID NO: 150 and a light chain variable region comprising the amino acid sequence of SEQ ID NO: 151. In some embodiments, the antibody of the antibody drug conjugate is EpAb2-6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 104, 105, 1181, 107, 108, and 109, respectively. In some embodiments, the antibody of the antibody drug conjugate comprises CDR-H1 comprising the amino acid sequence of SEQ ID NO: 1181.

In some embodiments, an antibody-drug conjugate provided herein binds to CD352. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 152, 153, 154, 155, 156, and 157, respectively. In some embodiments, the antibody of the antibody drug conjugate comprises a heavy chain variable region comprising the amino acid sequence of SEQ ID NO: 158 and a light chain variable region comprising the amino acid sequence of SEQ ID NO: 159. In some embodiments, the antibody of the antibody drug conjugate is h20F3.

In some embodiments, an antibody-drug conjugate provided herein binds to CS 1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 160, 161, 162, 163, 164, and 165, respectively. In some embodiments, the antibody of the antibody drug conjugate comprises a heavy chain variable region comprising the amino acid sequence of SEQ ID NO: 166 and a light chain variable region comprising the amino acid sequence of SEQ ID NO: 167. In some embodiments, the antibody of the antibody drug conjugate is elotuzumab.

In some embodiments, an antibody-drug conjugate provided herein binds to CD38. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 168, 169, 170, 171, 172, and 173, respectively. In some embodiments, the antibody of the antibody drug conjugate comprises a heavy chain variable region comprising the amino acid sequence of SEQ ID NO: 174 and a light chain variable region comprising the amino acid sequence of SEQ ID NO: 175. In some embodiments, the antibody of the antibody drug conjugate is daratumumab.

In some embodiments, an antibody-drug conjugate provided herein binds to CD25. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 176, 177, 178, 179, 180, and 181, respectively. In some embodiments, the antibody of the antibody drug conjugate comprises a heavy chain variable region comprising the amino acid sequence of SEQ ID NO: 182 and a light chain variable region comprising the amino acid sequence of SEQ ID NO: 183. In some embodiments, the antibody of the antibody drug conjugate is daclizumab.

In some embodiments, an antibody-drug conjugate provided herein binds to ADAM9. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 184, 185, 186, 187, 188, and 189, respectively. In some embodiments, the antibody of the antibody drug conjugate comprises a heavy chain variable region comprising the amino acid sequence of SEQ ID NO: 190 and a light chain variable region comprising the amino acid sequence of SEQ ID NO: 191. In some embodiments, the antibody of the antibody drug conjugate is chMAbA9-A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 192, 193, 194, 195, 196, and 197, respectively. In some embodiments, the antibody of the antibody drug conjugate comprises a heavy chain variable region comprising the amino acid sequence of SEQ ID NO: 198 and a light chain variable region comprising the amino acid sequence of SEQ ID NO: 199. In some embodiments, the antibody of the antibody drug conjugate is hMAbA9-A. In some embodiments, an antibody-drug conjugate provided herein binds to ADAM9. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1183, 185, 186, 187, 188, and 189, respectively. In some embodiments, an antibody-drug conjugate provided herein binds to ADAM9. In some embodiments, the antibody of the antibody drug conjugate comprises CDR-H1 comprising the amino acid sequences of SEQ ID NO: 1183. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1184, 193, 194, 1185, 196, and 197, respectively. In some embodiments, the antibody of the antibody drug conjugate comprises CDR-H1 comprising the amino acid sequences of SEQ ID NO: 1184. In some embodiments, the antibody of the antibody drug conjugate comprises CDR-L1 comprising the amino acid sequences of SEQ ID NO: 1185.

In some embodiments, an antibody-drug conjugate provided herein binds to CD59. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 200, 201, 202, 203, 204, and 205, respectively. In some embodiments, the antibody of the antibody drug conjugate comprises a heavy chain variable region comprising the amino acid sequence of SEQ ID NO: 206 and a light chain variable region comprising the amino acid sequence of SEQ ID NO: 207. In some embodiments, an antibody-drug conjugate provided herein binds to CD59. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1186, 1187, 202, 203, 204, and 205, respectively. In some embodiments, the antibody of the antibody drug conjugate comprises CDR-H1 comprising the amino acid sequence of SEQ ID NO: 1186. In some embodiments, the antibody of the antibody drug conjugate comprises CDR-H2 comprising the amino acid sequence of SEQ ID NO: 1187. In some embodiments, the antibody of the antibody drug conjugate comprises a heavy chain variable region comprising the amino acid sequence of SEQ ID NO: 1188 and a light chain variable region comprising the amino acid sequence of SEQ ID NO: 207.

In some embodiments, an antibody-drug conjugate provided herein binds to CD25. In some embodiments, the antibody of the antibody drug conjugate is Clone123.

In some embodiments, an antibody-drug conjugate provided herein binds to CD229. In some embodiments, the antibody of the antibody drug conjugate is h8A10.

In some embodiments, an antibody-drug conjugate provided herein binds to CD19. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 208, 209, 210, 211, 212, and 213, respectively. In some embodiments, the antibody of the antibody drug conjugate comprises a heavy chain variable region comprising the amino acid sequence of SEQ ID NO: 214 and a light chain variable region comprising the amino acid sequence of SEQ ID NO: 215. In some embodiments, the anti-CD 19 antibody comprises a heavy chain comprising an amino acid sequence that is at least 80%, at least 85%, at least 90%, at least 95%, at least 98%, or 99% identical to the amino acid sequence of SEQ ID NO: 1175 and a light chain comprising an amino acid sequence that is at least 80% at least 85%, at least 90%, at least 95%, at least 98%, or 99% identical to the amino acid sequence of SEQ ID NO: 1176. In some embodiments, the antibody of the antibody drug conjugate is denintuzumab, which is also known as hBU12. See WO2009052431.

In some embodiments, an antibody-drug conjugate provided herein binds to CD70. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 216, 217, 218, 219, 220, and 221, respectively. In some embodiments, the antibody of the antibody drug conjugate comprises a heavy chain variable region comprising the amino acid sequence of SEQ ID NO: 222 and a light chain variable region comprising the amino acid sequence of SEQ ID NO: 223. In some embodiments, the antibody of the antibody drug conjugate is vorsetuzumab. In some cases, an antibody provided herein binds to CD70. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1169, 1170, 1171, 1172, 1173 and 1174, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1169, 1170, 1171, 1172, 1173 and 1174, respectively.

In some embodiments, an antibody-drug conjugate provided herein binds to B7H4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 224, 225, 226, 227, 228, and 229, respectively. In some embodiments, the antibody of the antibody drug conjugate comprises a heavy chain variable region comprising the amino acid sequence of SEQ ID NO: 230 and a light chain variable region comprising the amino acid sequence of SEQ ID NO: 231. In some embodiments, the antibody of the antibody drug conjugate is mirzotamab.

In some cases, an antibody provided herein binds to B7H4. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 77-82, SEQ ID NOs: 91-96, SEQ ID NOs: 99-104, SEQ ID NOs: 985-990, SEQ ID NOs: 993-998, SEQ ID NOs:1001-128, SEQ ID NOs: 1009-1014, SEQ ID NOs: 1017-1022, SEQ ID NOs: 1025-1030, SEQ ID NOs: 1033-1038, SEQ ID NOs: 1041-1046, SEQ ID NOs: 1049-1054, SEQ ID NOs: 1057-1062, SEQ ID NOs: 1065-1070, SEQ ID NOs: 1073-1078, SEQ ID NOs: 1081-1086, SEQ ID NOs: 1089-1094, SEQ ID NOs: 1097-1102, SEQ ID NOs: 1105-1110, SEQ ID NOs: 1113-1118, and SEQ ID NOs: 1121-1126. In some cases, the antibody comprises a set of CDR sequences (CDR-H1, CDR-H2, CDR-H3, CDR-L1, CDR-L2, and CDR-L3, respectively) each comprising at most one mutation relative to an amino acid sequence from a set of amino acid sequences selected from the group consisting of SEQ ID NOs: 77-82, SEQ ID NOs: 91-96, SEQ ID NOs: 99 104, SEQ ID NOs: 985-990, SEQ ID NOs: 993-998, SEQ ID NOs: 1001-1006, SEQ ID NOs: 1009-1014, SEQ ID NOs: 1017-1022, SEQ ID NOs: 1025-1030, SEQ ID NOs: 1033-1038, SEQ ID NOs: 1041-1046, SEQ ID NOs: 1049-1054, SEQ ID NOs: 1057-1062, SEQ ID NOs: 1065-1070, SEQ ID NOs: 1073-1078, SEQ ID NOs: 1081-1086, SEQ ID NOs: 1089-1094, SEQ ID NOs: 1097-1102, SEQ ID NOs: 1105-1110, SEQ ID NOs: 1113-1118, and SEQ ID NOs: 1121-1126. In some cases, the anti-B7H4 antibody comprises a heavy chain and a light chain comprising amino acid sequences that are at least 80%, at least 85%, at least 90%, at least 95%, at least 98%, at least 99%, or at least 100% identical to the amino acid sequences of SEQ ID NO: 963 and 87, SEQ ID NO: 964 and 87, SEQ ID NO: 966 and 90, SEQ ID NO: 967 and 90, SEQ ID NO: 1129 and 1130, SEQ ID NO: 1131 11321131 and 1132, SEQ ID NO: 1133 and 1134, SEQ ID NO: 1135 and 1136, SEQ ID NO: 1137 and 1138, SEQ ID NO: 1139 and 1140, SEQ ID NO: 1141 and 1142, SEQ ID NO: 1143 and 1144, SEQ ID NO: 1145 and 1146, SEQ ID NO: 1147 and 1148, SEQ ID NO: 1149 and 1150, SEQ ID NO: 1151 and 1152, SEQ ID NO: 1153 and 1154, SEQ ID NO: 1155 and 1156, SEQ ID NO: 1157 and 1158, SEQ ID NO: 1159 and 1160, SEQ ID NO: 1161 and 1162, SEQ ID NO: 1163 and 1164, SEQ ID NO: 1165 and 1166, or SEQ ID NO: 1167 and 1168, respectively. In some embodiments, the anti-B7H4 antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 961 and 962, SEQ ID NO: 975 and 98, SEQ ID NO: 983 and 984, SEQ ID NO: 991 and 992, SEQ ID NO: 999 and 1000, SEQ ID NO: 1007 and 1008, SEQ ID NO: 1015 and 1016, SEQ ID NO: 1031 and 1032, SEQ ID NO: 1039 and 1040, SEQ ID NO: 1047 and 1048, SEQ ID NO: 1055 and 1056, SEQ ID NO: 1063 and 1064, SEQ ID NO: 1071 and 1072, SEQ ID NO: 1079 and 1080, SEQ ID NO: 1087 and 1088, SEQ ID NO: 1095 and 1096, SEQ ID NO: 1103 and 1104, SEQ ID NO: 1111 and 1112, SEQ ID NO: 1119 and 1120, or SEQ ID NO: 1127 and 1128, respectively.

In some embodiments, an antibody-drug conjugate provided herein binds to CD138. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 232, 233, 234, 235, 236, and 237, respectively. In some embodiments, the antibody of the antibody drug conjugate comprises a heavy chain variable region comprising the amino acid sequence of SEQ ID NO: 238 and a light chain variable region comprising the amino acid sequence of SEQ ID NO: 239. In some embodiments, the antibody of the antibody drug conjugate is indatuxumab.

In some embodiments, an antibody-drug conjugate provided herein binds to CD 166. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 240, 241, 242, 243, 244, and 245, respectively. In some embodiments, the antibody of the antibody drug conjugate comprises a heavy chain variable region comprising the amino acid sequence of SEQ ID NO: 246 and a light chain variable region comprising the amino acid sequence of SEQ ID NO: 247. In some embodiments, the antibody of the antibody drug conjugate is praluzatamab.

In some embodiments, an antibody-drug conjugate provided herein binds to CD51. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 248, 249, 250, 251, 252, and 253, respectively. In some embodiments, the antibody of the antibody drug conjugate comprises a heavy chain variable region comprising the amino acid sequence of SEQ ID NO: 254 and a light chain variable region comprising the amino acid sequence of SEQ ID NO: 255. In some embodiments, the antibody of the antibody drug conjugate is intetumumab.

In some embodiments, an antibody-drug conjugate provided herein binds to CD56. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 256, 257, 258, 259, 260, and 261, respectively. In some embodiments, the antibody of the antibody drug conjugate comprises a heavy chain variable region comprising the amino acid sequence of SEQ ID NO: 262 and a light chain variable region comprising the amino acid sequence of SEQ ID NO: 263. In some embodiments, the antibody of the antibody drug conjugate is lorvotuzumab.

In some embodiments, an antibody-drug conjugate provided herein binds to CD74. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 264, 265, 266, 267, 268, and 269, respectively. In some embodiments, the antibody of the antibody drug conjugate comprises a heavy chain variable region comprising the amino acid sequence of SEQ ID NO: 270 and a light chain variable region comprising the amino acid sequence of SEQ ID NO: 271. In some embodiments, the antibody of the antibody drug conjugate is milatuzumab.

In some embodiments, an antibody-drug conjugate provided herein binds to CEACAM5. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 272, 273 274, 275, 276, and 277, respectively. In some embodiments, the antibody of the antibody drug conjugate comprises a heavy chain variable region comprising the amino acid sequence of SEQ ID NO: 278 and a light chain variable region comprising the amino acid sequence of SEQ ID NO: 279. In some embodiments, the antibody of the antibody drug conjugate is labetuzumab.

In some embodiments, an antibody-drug conjugate provided herein binds to CanAg. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 280, 281, 282, 283, 284, and 285, respectively. In some embodiments, the antibody of the antibody drug conjugate comprises a heavy chain variable region comprising the amino acid sequence of SEQ ID NO: 286 and a light chain variable region comprising the amino acid sequence of SEQ ID NO: 287. In some embodiments, the antibody of the antibody drug conjugate is cantuzumab.

In some embodiments, an antibody-drug conjugate provided herein binds to DLL-3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 288, 289, 290, 291, 292, and 293, respectively. In some embodiments, the antibody of the antibody drug conjugate comprises a heavy chain variable region comprising the amino acid sequence of SEQ ID NO: 294 and a light chain variable region comprising the amino acid sequence of SEQ ID NO: 295. In some embodiments, the antibody of the antibody drug conjugate is rovalpituzumab.

In some embodiments, an antibody-drug conjugate provided herein binds to DPEP-3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 296, 297, 298, 299, 300, and 301, respectively. In some embodiments, the antibody of the antibody drug conjugate comprises a heavy chain variable region comprising the amino acid sequence of SEQ ID NO: 302 and a light chain variable region comprising the amino acid sequence of SEQ ID NO: 303. In some embodiments, the antibody of the antibody drug conjugate is tamrintamab.

In some embodiments, an antibody-drug conjugate provided herein binds to EGFR′. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 304, 305, 306, 307, 308, and 309, respectively. In some embodiments, the antibody of the antibody drug conjugate comprises a heavy chain variable region comprising the amino acid sequence of SEQ ID NO: 310 and a light chain variable region comprising the amino acid sequence of SEQ ID NO: 311. In some embodiments, the antibody of the antibody drug conjugate is laprituximab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 312, 313, 314, 315, 316, and 317, respectively. In some embodiments, the antibody of the antibody drug conjugate comprises a heavy chain variable region comprising the amino acid sequence of SEQ ID NO: 318 and a light chain variable region comprising the amino acid sequence of SEQ ID NO: 319. In some embodiments, the antibody of the antibody drug conjugate is losatuxizumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 320, 321, 322, 323, 324, and 325, respectively. In some embodiments, the antibody of the antibody drug conjugate comprises a heavy chain variable region comprising the amino acid sequence of SEQ ID NO: 326 and a light chain variable region comprising the amino acid sequence of SEQ ID NO: 327. In some embodiments, the antibody of the antibody drug conjugate is serclutamab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 328, 329, 330, 331, 332, and 333, respectively. In some embodiments, the antibody of the antibody drug conjugate comprises a heavy chain variable region comprising the amino acid sequence of SEQ ID NO: 334 and a light chain variable region comprising the amino acid sequence of SEQ ID NO: 335. In some embodiments, the antibody of the antibody drug conjugate is cetuximab.

In some embodiments, an antibody-drug conjugate provided herein binds to FRa. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 336, 337, 338, 339, 340, and 341, respectively. In some embodiments, the antibody of the antibody drug conjugate comprises a heavy chain variable region comprising the amino acid sequence of SEQ ID NO: 342 and a light chain variable region comprising the amino acid sequence of SEQ ID NO: 343. In some embodiments, the antibody of the antibody drug conjugate is mirvetuximab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 344, 345, 346, 347, 348, and 349, respectively. In some embodiments, the antibody of the antibody drug conjugate comprises a heavy chain variable region comprising the amino acid sequence of SEQ ID NO: 350 and a light chain variable region comprising the amino acid sequence of SEQ ID NO: 351. In some embodiments, the antibody of the antibody drug conjugate is farletuzumab.

In some embodiments, an antibody-drug conjugate provided herein binds to MUC-1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 352, 353, 354, 355, 356, and 357, respectively. In some embodiments, the antibody of the antibody drug conjugate comprises a heavy chain variable region comprising the amino acid sequence of SEQ ID NO: 358 and a light chain variable region comprising the amino acid sequence of SEQ ID NO: 359. In some embodiments, the antibody of the antibody drug conjugate is gatipotuzumab.

In some embodiments, an antibody-drug conjugate provided herein binds to mesothelin. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 360, 361, 362, 363, 364, and 365, respectively. In some embodiments, the antibody of the antibody drug conjugate comprises a heavy chain variable region comprising the amino acid sequence of SEQ ID NO: 366 and a light chain variable region comprising the amino acid sequence of SEQ ID NO: 367. In some embodiments, the antibody of the antibody drug conjugate is anetumab.

In some embodiments, an antibody-drug conjugate provided herein binds to ROR-1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 368, 369, 370, 371, 372, and 373, respectively. In some embodiments, the antibody of the antibody drug conjugate comprises a heavy chain variable region comprising the amino acid sequence of SEQ ID NO: 374 and a light chain variable region comprising the amino acid sequence of SEQ ID NO: 375. In some embodiments, the antibody of the antibody drug conjugate is zilovertamab.

In some embodiments, an antibody-drug conjugate provided herein binds to ASCT2.In some embodiments, an antibody-drug conjugate provided herein binds to B7H4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 376, 377, 378, 379, 380, and 381, respectively. In some embodiments, the antibody of the antibody drug conjugate comprises a heavy chain variable region comprising the amino acid sequence of SEQ ID NO: 382 and a light chain variable region comprising the amino acid sequence of SEQ ID NO: 383. In some embodiments, the antibody of the antibody drug conjugate is 20502. See WO2019040780.

In some embodiments, an antibody-drug conjugate provided herein binds to B7-H3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 384, 385, 386, 387, 388, and 389, respectively. In some embodiments, the antibody of the antibody drug conjugate comprises a heavy chain variable region comprising the amino acid sequence of SEQ ID NO: 390 and a light chain variable region comprising the amino acid sequence of SEQ ID NO: 391. In some embodiments, the antibody of the antibody drug conjugate is chAb-A (BRCA84D). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 392, 393, 394, 395, 396, and 397, respectively. In some embodiments, the antibody of the antibody drug conjugate comprises a heavy chain variable region comprising the amino acid sequence of SEQ ID NO: 398 and a light chain variable region comprising the amino acid sequence of SEQ ID NO: 399. In some embodiments, the antibody of the antibody drug conjugate is hAb-B. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 400, 401, 402, 403, 404, and 405, respectively. In some embodiments, the antibody of the antibody drug conjugate comprises a heavy chain variable region comprising the amino acid sequence of SEQ ID NO: 406 and a light chain variable region comprising the amino acid sequence of SEQ ID NO: 407. In some embodiments, the antibody of the antibody drug conjugate is hAb-C. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 408, 409, 410, 411, 412, and 413, respectively. In some embodiments, the antibody of the antibody drug conjugate comprises a heavy chain variable region comprising the amino acid sequence of SEQ ID NO: 414 and a light chain variable region comprising the amino acid sequence of SEQ ID NO: 415. In some embodiments, the antibody of the antibody drug conjugate is hAb-D. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 416, 417, 418, 419, 420, and 421, respectively. In some embodiments, the antibody of the antibody drug conjugate comprises a heavy chain variable region comprising the amino acid sequence of SEQ ID NO: 422 and a light chain variable region comprising the amino acid sequence of SEQ ID NO: 423. In some embodiments, the antibody of the antibody drug conjugate is chM30. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 424, 425, 426, 427, 428, and 429, respectively. In some embodiments, the antibody of the antibody drug conjugate comprises a heavy chain variable region comprising the amino acid sequence of SEQ ID NO: 430 and a light chain variable region comprising the amino acid sequence of SEQ ID NO: 431. In some embodiments, the antibody of the antibody drug conjugate is hM30-H1-L4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 432, 433, 434, 435, 436, and 437, respectively. In some embodiments, the antibody of the antibody drug conjugate comprises a heavy chain variable region comprising the amino acid sequence of SEQ ID NO: 438 and a light chain variable region comprising the amino acid sequence of SEQ ID NO: 439. In some embodiments, the antibody of the antibody drug conjugate is AbV_huAb18-v4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 440, 441, 442, 443, 444, and 445, respectively. In some embodiments, the antibody of the antibody drug conjugate comprises a heavy chain variable region comprising the amino acid sequence of SEQ ID NO: 446 and a light chain variable region comprising the amino acid sequence of SEQ ID NO: 447. In some embodiments, the antibody of the antibody drug conjugate is AbV_huAb3-v6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 448, 449, 450, 451, 452, and 453, respectively. In some embodiments, the antibody of the antibody drug conjugate comprises a heavy chain variable region comprising the amino acid sequence of SEQ ID NO: 454 and a light chain variable region comprising the amino acid sequence of SEQ ID NO: 455. In some embodiments, the antibody of the antibody drug conjugate is AbV_huAb3-v2.6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 456, 457, 458, 459, 460, and 461, respectively. In some embodiments, the antibody of the antibody drug conjugate comprises a heavy chain variable region comprising the amino acid sequence of SEQ ID NO: 462 and a light chain variable region comprising the amino acid sequence of SEQ ID NO: 463. In some embodiments, the antibody of the antibody drug conjugate is AbV_huAb13-v1-CR′. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 464, 465, 466, 467, 468, and 469, respectively. In some embodiments, the antibody of the antibody drug conjugate comprises a heavy chain variable region comprising the amino acid sequence of SEQ ID NO: 470 and a light chain variable region comprising the amino acid sequence of SEQ ID NO: 471. In some embodiments, the antibody of the antibody drug conjugate is 8H9-6m. In some embodiments, the antibody of the antibody drug conjugate comprises a heavy chain variable region comprising the amino acid sequence of SEQ ID NO: 472 and a light chain variable region comprising the amino acid sequence of SEQ ID NO: 473. In some embodiments, the antibody of the antibody drug conjugate is m8517. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 474, 475, 476, 477, 478, and 479, respectively. In some embodiments, the antibody of the antibody drug conjugate comprises a heavy chain variable region comprising the amino acid sequence of SEQ ID NO: 480 and a light chain variable region comprising the amino acid sequence of SEQ ID NO: 481. In some embodiments, the antibody of the antibody drug conjugate is TPP-5706. In some embodiments, the antibody of the antibody drug conjugate comprises a heavy chain variable region comprising the amino acid sequence of SEQ ID NO: 482 and a light chain variable region comprising the amino acid sequence of SEQ ID NO: 483. In some embodiments, the antibody of the antibody drug conjugate is TPP-6642. In some embodiments, the antibody of the antibody drug conjugate comprises a heavy chain variable region comprising the amino acid sequence of SEQ ID NO: 484 and a light chain variable region comprising the amino acid sequence of SEQ ID NO: 485. In some embodiments, the antibody of the antibody drug conjugate is TPP-6850. In some embodiments, an antibody-drug conjugate provided herein binds to B7-H3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 384, 1189, 1190, 1191, 1192, and 1193, respectively. In some embodiments, an antibody-drug conjugate provided herein binds to B7-H3. In some embodiments, the antibody of the antibody drug conjugate comprises CDR-H2, comprising the amino acid sequence of SEQ ID NO: 1189. In some embodiments, the antibody of the antibody drug conjugate comprises CDR-H3, comprising the amino acid sequence of SEQ ID NO: 1190. In some embodiments, the antibody of the antibody drug conjugate comprises CDR-L1, comprising the amino acid sequence of SEQ ID NO: 1191. In some embodiments, the antibody of the antibody drug conjugate comprises CDR-L2, comprising the amino acid sequence of SEQ ID NO: 1192. In some embodiments, the antibody of the antibody drug conjugate comprises CDR-L3, comprising the amino acid sequence of SEQ ID NO: 1193. In some embodiments, the antibody of the antibody drug conjugate is chAb-A (BRCA84D). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1194, 1195, 1196, 1197, 396, and 397, respectively. In some embodiments, the antibody of the antibody drug conjugate is chAb-A (BRCA84D). In some embodiments, the antibody of the antibody drug conjugate comprises CDR-H1, comprising the amino acid sequence of SEQ ID NO: 1194. In some embodiments, the antibody of the antibody drug conjugate comprises CDR-H2, comprising the amino acid sequence of SEQ ID NO: 1195. In some embodiments, the antibody of the antibody drug conjugate comprises CDR-H3, comprising the amino acid sequence of SEQ ID NO: 1196. In some embodiments, the antibody of the antibody drug conjugate comprises CDR-L1, comprising the amino acid sequence of SEQ ID NO: 1197. In some embodiments, the antibody of the antibody drug conjugate is hAb-B. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 400, 401, 402, 403, 404, and 1198, respectively. In some embodiments, the antibody of the antibody drug conjugate is hAb-B. In some embodiments, the antibody of the antibody drug conjugate comprises CDR-L3, comprising the amino acid sequence of SEQ ID NO: 1198. In some embodiments, the antibody of the antibody drug conjugate is hAb-C. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1199, 1200, 1201, 1202, 1203, and 1204, respectively. In some embodiments, the antibody of the antibody drug conjugate is hAb-C. In some embodiments, the antibody of the antibody drug conjugate comprises CDR-H1, comprising the amino acid sequence of SEQ ID NO: 1199. In some embodiments, the antibody of the antibody drug conjugate comprises CDR-H2, comprising the amino acid sequence of SEQ ID NO: 1200. In some embodiments, the antibody of the antibody drug conjugate comprises CDR-H3, comprising the amino acid sequence of SEQ ID NO: 1201. In some embodiments, the antibody of the antibody drug conjugate comprises CDR-L1, comprising the amino acid sequence of SEQ ID NO: 1202. In some embodiments, the antibody of the antibody drug conjugate comprises CDR-L2, comprising the amino acid sequence of SEQ ID NO: 1203. In some embodiments, the antibody of the antibody drug conjugate comprises CDR-L3, comprising the amino acid sequence of SEQ ID NO: 1204. In some embodiments, the antibody of the antibody drug conjugate is hM30-H1-L4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1205, 433, 434, 435, 436, and 437, respectively. In some embodiments, the antibody of the antibody drug conjugate comprises CDR-H1, comprising the amino acid sequence of SEQ ID NO: 1205. In some embodiments, the antibody of the antibody drug conjugate is hM30-H1-L4. In some embodiments, the antibody of the antibody drug conjugate comprises a heavy chain variable region comprising the amino acid sequence of SEQ ID NO: 438 and a light chain variable region comprising the amino acid sequence of SEQ ID NO: 1206. In some embodiments, the antibody of the antibody drug conjugate is AbV_huAb18-v4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1207, 441, 1208, 443, 444, and 445, respectively. In some embodiments, the antibody of the antibody drug conjugate is AbV_huAb18-v4. In some embodiments, the antibody of the antibody drug conjugate comprises CDR-H1, comprising the amino acid sequence of SEQ ID NO: 1207. In some embodiments, the antibody of the antibody drug conjugate comprises CDR-H3, comprising the amino acid sequence of SEQ ID NO: 1208. In some embodiments, the antibody of the antibody drug conjugate is AbV_huAb3-v6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1209, 449, 450, 451, 452, and 453, respectively. In some embodiments, the antibody of the antibody drug conjugate comprises CDR-H1, comprising the amino acid sequence of SEQ ID NO: 1209. In some embodiments, the antibody of the antibody drug conjugate comprises a heavy chain variable region comprising the amino acid sequence of SEQ ID NO: 454 and a light chain variable region comprising the amino acid sequence of SEQ ID NO: 1210. In some embodiments, the antibody of the antibody drug conjugate is AbV_huAb3-v2.6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 1211, 457, 458, 459, 460, and 461, respectively. In some embodiments, the antibody of the antibody drug conjugate comprises CDR-H1, comprising the amino acid sequence of SEQ ID NO: 1211. In some embodiments, the antibody of the antibody drug conjugate comprises a heavy chain variable region comprising the amino acid sequence of SEQ ID NO: 462 and a light chain variable region comprising the amino acid sequence of SEQ ID NO: 1212. In some embodiments, the antibody of the antibody drug conjugate is AbV_huAb13-v1-CR′. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 464, 1213, 466, 467, 468, and 1214, respectively. In some embodiments, the antibody of the antibody drug conjugate comprises CDR-H2, comprising the amino acid sequence of SEQ ID NO: 1213. In some embodiments, the antibody of the antibody drug conjugate comprises CDR-13, comprising the amino acid sequence of SEQ ID NO: 1214.

In some embodiments, an antibody-drug conjugate provided herein binds to CDCP1. In some embodiments, the antibody of the antibody drug conjugate is 10D7.

In some embodiments, an antibody-drug conjugate provided herein binds to HER3. In some embodiments, the antibody of the antibody drug conjugate comprises a heavy chain comprising the amino acid sequence of SEQ ID NO: 486 and a light chain comprising the amino acid sequence of SEQ ID NO: 487. In some embodiments, the antibody of the antibody drug conjugate is patritumab. In some embodiments, the antibody of the antibody drug conjugate comprises a heavy chain comprising the amino acid sequence of SEQ ID NO: 488 and a light chain comprising the amino acid sequence of SEQ ID NO: 489. In some embodiments, the antibody of the antibody drug conjugate is seribantumab. In some embodiments, the antibody of the antibody drug conjugate comprises a heavy chain comprising the amino acid sequence of SEQ ID NO: 490 and a light chain comprising the amino acid sequence of SEQ ID NO: 491. In some embodiments, the antibody of the antibody drug conjugate is elgemtumab. In some embodiments, the antibody of the antibody drug conjugate comprises a heavy chain the amino acid sequence of SEQ ID NO: 492 and a light chain comprising the amino acid sequence of SEQ ID NO: 493. In some embodiments, the antibody of the antibody drug conjugate is lumretuzumab.

In some embodiments, an antibody-drug conjugate provided herein binds to RON. In some embodiments, the antibody of the antibody drug conjugate is Zt/g4.

In some embodiments, an antibody-drug conjugate provided herein binds to claudin-2.

In some embodiments, an antibody-drug conjugate provided herein binds to HLA-G.

In some embodiments, an antibody-drug conjugate provided herein binds to PTK7. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 494, 495, 496, 497, 498, and 499, respectively. In some embodiments, the antibody of the antibody drug conjugate comprises a heavy chain variable region comprising the amino acid sequence of SEQ ID NO: 500 and a light chain variable region comprising the amino acid sequence of SEQ ID NO: 501. In some embodiments, the antibody of the antibody drug conjugate is PTK7 mab 1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 502, 503, 504, 505, 506, and 507, respectively. In some embodiments, the antibody of the antibody drug conjugate comprises a heavy chain variable region comprising the amino acid sequence of SEQ ID NO: 508 and a light chain variable region comprising the amino acid sequence of SEQ ID NO: 509. In some embodiments, the antibody of the antibody drug conjugate is PTK7 mab 2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 510, 511, 512, 513, 514, and 515, respectively. In some embodiments, the antibody of the antibody drug conjugate comprises a heavy chain variable region comprising the amino acid sequence of SEQ ID NO: 516 and a light chain variable region comprising the amino acid sequence of SEQ ID NO: 517. In some embodiments, the antibody of the antibody drug conjugate is PTK7 mab 3.

In some embodiments, an antibody-drug conjugate provided herein binds to LIV1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 518, 519, 520, 521, 522, and 523, respectively. In some embodiments, the antibody of the antibody drug conjugate comprises a heavy chain variable region comprising the amino acid sequence of SEQ ID NO: 524 and a light chain variable region comprising the amino acid sequence of SEQ ID NO: 525. In some embodiments, the antibody of the antibody drug conjugate is ladiratuzumab, which is also known as hLIV22 and hglg. See WO2012078668.

In some embodiments, an antibody-drug conjugate provided herein binds to avb6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 526, 527, 528, 529, 530, and 531, respectively. In some embodiments, the antibody of the antibody drug conjugate comprises a heavy chain variable region comprising the amino acid sequence of SEQ ID NO: 532 and a light chain variable region comprising the amino acid sequence of SEQ ID NO: 533. In some embodiments, the antibody of the antibody drug conjugate is h2A2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 534, 535, 536, 537, 538, and 539, respectively. In some embodiments, the antibody of the antibody drug conjugate comprises a heavy chain variable region comprising the amino acid sequence of SEQ ID NO: 540 and a light chain variable region comprising the amino acid sequence of SEQ ID NO: 541. In some embodiments, the antibody of the antibody drug conjugate is h15H3.

In some cases, an antibody provided herein binds to avB6. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 941, 942, 943, 944, 945, and 946, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 941, 942, 943, 944, 945, and 946, respectively. In some embodiments, the anti-H2A2 antibody comprises a heavy chain variable region comprising an amino acid sequence that is at least 80%, at least 85%, at least 90%, at least 95%, at least 98%, or 99% identical to the amino acid sequence of SEQ ID NO: 947 and a light chain variable region comprising an amino acid sequence that is at least 80% at least 85%, at least 90%, at least 95%, at least 98%, or 99% identical to the amino acid sequence of SEQ ID NO: 948. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 949 or 950 and a light chain comprising an amino acid sequence that is at least 80% at least 85%, at least 90%, at least 95%, at least 98%, or 99% identical to the amino acid sequence of SEQ ID NO: 951. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 952 or 953 and a light chain comprising an amino acid sequence that is at least 80% at least 85%, at least 90%, at least 95%, at least 98%, or 99% identical to the amino acid sequence of SEQ ID NO: 954.

In some embodiments, an antibody-drug conjugate provided herein binds to CD48. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 542, 543, 544, 545, 546, and 547, respectively. In some embodiments, the antibody of the antibody drug conjugate comprises a heavy chain variable region comprising the amino acid sequence of SEQ ID NO: 548 and a light chain variable region comprising the amino acid sequence of SEQ ID NO: 549. In some embodiments, the antibody of the antibody drug conjugate is hMEM102. See WO2016149535.

In some embodiments, an antibody-drug conjugate provided herein binds to PD-L1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 550, 551, 552, 553, 554, and 555, respectively. In some embodiments, the antibody of the antibody drug conjugate comprises a heavy chain variable region comprising the amino acid sequence of SEQ ID NO: 556 and a light chain variable region comprising the amino acid sequence of SEQ ID NO: 557. In some embodiments, the antibody of the antibody drug conjugate is SG-559-01 LALA mAb.

In some cases, an anti-PDL1 antibody comprises CDR-H1, CDR-H2, CDR-H3, CDR-L1, CDR-L2, and CDR-L3 comprising at least 80% sequence identity, at least 85% sequence identity, at least 90% sequence identity, at least 95%, at least 98%, or at least 99% sequence identity to the amino acid sequences of SEQ ID NOs: 902, 903, 903, 904, 905, 906, and 18 respectively. In some cases, the antibody comprises CDR-H1, CDR-H2, CDR-H3, CDR-L1, CDR-L2, and CDR-L3 each comprising at most one mutation relative to the amino acid sequences of SEQ ID NOs: 902, 903, 903, 904, 905, 906, and 907, respectively.

In some embodiments, the antibody comprises a heavy chain comprising an amino acid sequence that is at least 80%, at least 85%, at least 90%, at least 95%, at least 98%, or 99% identical to the amino acid sequence of any one of SEQ ID NO: 890-893 and a light chain comprising an amino acid sequence that is at least 95% at least 96%, at least 97%, at least 98%, at least 99%, or 100% identical to the amino acid sequence of SEQ ID NO: 894. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 890 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 891 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 892 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 893 and a light chain comprising an amino acid sequence that is at least 95% at least 96%, at least 97%, at least 98%, at least 99%, or 100% identical to the amino acid sequence of SEQ ID NO: 5.

In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 895-898 and a light chain variable region comprising an amino acid sequence that is at least 95% at least 96%, at least 97%, at least 98%, at least 99%, or 100% identical to the amino acid sequence of SEQ ID NO: 899. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 895 and a light chain variable region comprising an amino acid sequence that is at least 95% at least 96%, at least 97%, at least 98%, at least 99%, or 100% identical to the amino acid sequence of SEQ ID NO: 899. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 896 and a light chain variable region comprising an amino acid sequence that is at least 95% at least 96%, at least 97%, at least 98%, at least 99%, or 100% identical to the amino acid sequence of SEQ ID NO: 899.In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 897 and a light chain variable region comprising an amino acid sequence that is at least 95% at least 96%, at least 97%, at least 98%, at least 99%, or 100% identical to the amino acid sequence of SEQ ID NO: 899.In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 898 and a light chain variable region comprising an amino acid sequence that is at least 95% at least 96%, at least 97%, at least 98%, at least 99%, or 100% identical to the amino acid sequence of SEQ ID NO: 899.

In some embodiments, the antibody comprises a heavy chain variable region comprising an amino acid sequence that is at least 80%, at least 85%, at least 90%, at least 95%, at least 98%, or 99% identical to the amino acid sequence of SEQ ID NO: 908 and a light chain variable region comprising an amino acid sequence that is at least 95% at least 96%, at least 97%, at least 98%, at least 99%, or 100% identical to the amino acid sequence of SEQ ID NO: 909.

In some embodiments, an antibody provided herein binds to EphA2. In some embodiments, the antibody comprises CDR-H1, CDR-H2, CDR-H3, CDR-L1, CDR-L2, and CDR-L3 comprising the amino acid sequences that are at least 80%, at least 85%, at least 90%, at least 95%, at least 98%, or at least 99% identical to the amino acid sequences of SEQ ID NOs: 910, 911, 912, 913, 914, and 915, respectively.

In some embodiments, the anti-EphA2 antibody comprises a heavy chain variable region comprising an amino acid sequence that is at least 80%, at least 85%, at least 90%, at least 95%, at least 98%, or at least 99% identical to the amino acid sequence of SEQ ID NO: 916 and a light chain variable region comprising an amino acid sequence that is at least 80%, at least 85%, at least 90%, at least 95%, at least 98%, or at least 99% identical to the amino acid sequence of SEQ ID NO: 917. In some embodiments, the anti-EphA2 antibody comprises a heavy chain comprising an amino acid sequence that is at least 80%, at least 85%, at least 90%, at least 95%, at least 98%, or at least 99% identical to the amino acid sequence of SEQ ID NO: 918 or SEQ ID NO: 919 and a light chain comprising an amino acid sequence that is at least 80%, at least 85%, at least 90%, at least 95%, at least 98%, or at least 99% identical to the amino acid sequence of the amino acid sequence of SEQ ID NO: 920. In some embodiments, the anti-EphA2 antibody comprises a heavy chain comprising the amino acid sequence that is at least 80%, at least 85%, at least 90%, at least 95%, at least 98%, or at least 99% identical to the amino acid sequence of SEQ ID NO: 921 or SEQ ID NO: 922 and a light chain comprising the amino acid sequence of SEQ ID NO: 923. In some embodiments, the anti-EphA2 antibody comprises a heavy chain that is at least 80%, at least 85%, at least 90%, at least 95%, at least 98%, or at least 99% identical to the amino acid sequence of SEQ ID NO: 924 or SEQ ID NO: 925 and a light chain comprising the amino acid sequence of SEQ ID NO: 926. In some embodiments, the antibody is h 1 C₁ or 1C₁.

In some embodiments, the anti-EphA2 antibody comprises a heavy chain variable region comprising an amino acid sequence that is at least 80%, at least 85%, at least 90%, at least 95%, at least 98%, or 99% identical to the amino acid sequence of SEQ ID NO: 916 and a light chain variable region comprising an amino acid sequence that is at least 80% at least 85%, at least 90%, at least 95%, at least 98%, or 99% identical to the amino acid sequence of SEQ ID NO: 917.

In some embodiments, an antibody-drug conjugate provided herein binds to IGF-1R′. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 558, 559, 560, 561, 562, and 563, respectively. In some embodiments, the antibody of the antibody drug conjugate comprises a heavy chain variable region comprising the amino acid sequence of SEQ ID NO: 564 and a light chain variable region comprising the amino acid sequence of SEQ ID NO: 565. In some embodiments, the antibody of the antibody drug conjugate is cixutumumab.

In some embodiments, an antibody-drug conjugate provided herein binds to claudin-18.2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 566, 567, 568, 569, 570, and 571, respectively. In some embodiments, the antibody of the antibody drug conjugate comprises a heavy chain variable region comprising the amino acid sequence of SEQ ID NO: 572 and a light chain variable region comprising the amino acid sequence of SEQ ID NO: 573. In some embodiments, the antibody of the antibody drug conjugate is zolbetuximab (175D10). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 574, 575, 576, 577, 578, and 579, respectively. In some embodiments, the antibody of the antibody drug conjugate comprises a heavy chain variable region comprising the amino acid sequence of SEQ ID NO: 580 and a light chain variable region comprising the amino acid sequence of SEQ ID NO: 581. In some embodiments, the antibody of the antibody drug conjugate is 163E12.

In some embodiments, an antibody-drug conjugate provided herein binds to Nectin-4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 582, 583, 584, 585, 586, and 587, respectively. In some embodiments, the antibody of the antibody drug conjugate comprises a heavy chain variable region comprising the amino acid sequence of SEQ ID NO: 588 and a light chain variable region comprising the amino acid sequence of SEQ ID NO: 589. In some embodiments, the antibody of the antibody drug conjugate is enfortumab. See WO 2012047724.

In some embodiments, an antibody-drug conjugate provided herein binds to SLTRK6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 590, 591, 592, 593, 594, and 595, respectively. In some embodiments, the antibody of the antibody drug conjugate comprises a heavy chain variable region comprising the amino acid sequence of SEQ ID NO: 596 and a light chain variable region comprising the amino acid sequence of SEQ ID NO: 597. In some embodiments, the antibody of the antibody drug conjugate is sirtratumab.

In some embodiments, an antibody-drug conjugate provided herein binds to CD228. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 598, 599, 600, 601, 602, and 603, respectively. In some embodiments, the antibody of the antibody drug conjugate comprises a heavy chain variable region comprising the amino acid sequence of SEQ ID NO: 604 and a light chain variable region comprising the amino acid sequence of SEQ ID NO: 605. In some embodiments, the antibody of the antibody drug conjugate is hL49. See WO 2020/163225.

In some cases, an antibody provided herein binds to CD228. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 927, 928, 929, 930, 931, and 932, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 927, 928, 929, 930, 931, and 932, respectively. In some embodiments, the anti-CD228 antibody comprises a heavy chain variable region comprising an amino acid sequence that is at least 80%, at least 85%, at least 90%, at least 95%, at least 98%, or 99% identical to the amino acid sequence of SEQ ID NO: 933 and a light chain variable region comprising an amino acid sequence that is at least 80% at least 85%, at least 90%, at least 95%, at least 98%, or 99% identical to the amino acid sequence of SEQ ID NO: 934. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 935 or 936 and a light chain comprising an amino acid sequence that is at least 80% at least 85%, at least 90%, at least 95%, at least 98%, or 99% identical to the amino acid sequence of SEQ ID NO: 937. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 938 or 939 and a light chain comprising an amino acid sequence that is at least 80% at least 85%, at least 90%, at least 95%, at least 98%, or 99% identical to the amino acid sequence of SEQ ID NO: 940.

In some embodiments, an antibody-drug conjugate provided herein binds to CD142 (tissue factor; TF). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 606, 607, 608, 609, 610, and 611, respectively. In some embodiments, the antibody of the antibody drug conjugate comprises a heavy chain variable region comprising the amino acid sequence of SEQ ID NO: 612 and a light chain variable region comprising the amino acid sequence of SEQ ID NO: 613. In some embodiments, the antibody of the antibody drug conjugate is tisotumab. See WO 2010/066803.

In some embodiments, an antibody-drug conjugate provided herein binds to STn. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 614, 615, 616, 617, 618, and 619, respectively. In some embodiments, the antibody of the antibody drug conjugate comprises a heavy chain variable region comprising the amino acid sequence of SEQ ID NO: 620 and a light chain variable region comprising the amino acid sequence of SEQ ID NO: 621. In some embodiments, the antibody of the antibody drug conjugate is h2G12.

In some embodiments, an antibody-drug conjugate provided herein binds to CD20. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 622, 623, 624, 625, 626, and 627, respectively. In some embodiments, the antibody of the antibody drug conjugate comprises a heavy chain variable region comprising the amino acid sequence of SEQ ID NO: 628 and a light chain variable region comprising the amino acid sequence of SEQ ID NO: 629. In some embodiments, the antibody of the antibody drug conjugate is rituximab.

In some embodiments, an antibody-drug conjugate provided herein binds to HER2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 630, 631, 632, 633, 634, and 635, respectively. In some embodiments, the antibody of the antibody drug conjugate comprises a heavy chain variable region comprising the amino acid sequence of SEQ ID NO: 636 and a light chain variable region comprising the amino acid sequence of SEQ ID NO: 637. In some embodiments, the antibody of the antibody drug conjugate is trastuzumab.

In some embodiments, an antibody-drug conjugate provided herein binds to FLT3.

In some embodiments, an antibody-drug conjugate provided herein binds to CD46.

In some embodiments, an antibody-drug conjugate provided herein binds to GloboH.

In some embodiments, an antibody-drug conjugate provided herein binds to AG7.

In some embodiments, an antibody-drug conjugate provided herein binds to mesothelin.

In some embodiments, an antibody-drug conjugate provided herein binds to FCRH5.

In some embodiments, an antibody-drug conjugate provided herein binds to ETBR.

In some embodiments, an antibody-drug conjugate provided herein binds to Tim-1.

In some embodiments, an antibody-drug conjugate provided herein binds to SLC44A4.

In some embodiments, an antibody-drug conjugate provided herein binds to ENPP3.

In some embodiments, an antibody-drug conjugate provided herein binds to CD37.

In some embodiments, an antibody-drug conjugate provided herein binds to CA9.

In some embodiments, an antibody-drug conjugate provided herein binds to Notch3.

In some embodiments, an antibody-drug conjugate provided herein binds to EphA2.

In some embodiments, an antibody-drug conjugate provided herein binds to TRFC.

In some embodiments, an antibody-drug conjugate provided herein binds to PSMA.

In some embodiments, an antibody-drug conjugate provided herein binds to LRRC15.

In some embodiments, an antibody-drug conjugate provided herein binds to 5T4.

In some embodiments, an antibody-drug conjugate provided herein binds to CD79b. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 638, 639, 640, 641, 642, and 643, respectively. In some embodiments, the antibody of the antibody drug conjugate comprises a heavy chain variable region comprising the amino acid sequence of SEQ ID NO: 644 and a light chain variable region comprising the amino acid sequence of SEQ ID NO: 645. In some embodiments, the antibody of the antibody drug conjugate is polatuzumab.

In some embodiments, an antibody-drug conjugate provided herein binds to NaPi2B. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 646, 647, 648, 649, 650, and 651, respectively. In some embodiments, the antibody of the antibody drug conjugate comprises a heavy chain variable region comprising the amino acid sequence of SEQ ID NO: 652 and a light chain variable region comprising the amino acid sequence of SEQ ID NO: 653. In some embodiments, the antibody of the antibody drug conjugate is lifastuzumab.

In some embodiments, an antibody-drug conjugate provided herein binds to Muc16. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 654, 655, 656, 657, 658, and 659, respectively. In some embodiments, the antibody of the antibody drug conjugate comprises a heavy chain variable region comprising the amino acid sequence of SEQ ID NO: 660 and a light chain variable region comprising the amino acid sequence of SEQ ID NO: 661. In some embodiments, the antibody of the antibody drug conjugate is sofituzumab.

In some embodiments, an antibody-drug conjugate provided herein binds to STEAP1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 662, 663, 664, 665, 666, and 667, respectively. In some embodiments, the antibody of the antibody drug conjugate comprises a heavy chain variable region comprising the amino acid sequence of SEQ ID NO: 668 and a light chain variable region comprising the amino acid sequence of SEQ ID NO: 669. In some embodiments, the antibody of the antibody drug conjugate is vandortuzumab.

In some embodiments, an antibody-drug conjugate provided herein binds to BCMA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 670, 671, 672, 673, 674, and 675, respectively. In some embodiments, the antibody of the antibody drug conjugate comprises a heavy chain variable region comprising the amino acid sequence of SEQ ID NO: 676 and a light chain variable region comprising the amino acid sequence of SEQ ID NO: 677. In some embodiments, the antibody of the antibody drug conjugate is belantamab.

In some embodiments, an antibody-drug conjugate provided herein binds to c-Met. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 678, 679, 680, 681, 682, and 683, respectively. In some embodiments, the antibody of the antibody drug conjugate comprises a heavy chain variable region comprising the amino acid sequence of SEQ ID NO: 684 and a light chain variable region comprising the amino acid sequence of SEQ ID NO: 685. In some embodiments, the antibody of the antibody drug conjugate is telisotuzumab.

In some embodiments, an antibody-drug conjugate provided herein binds to EGFR′. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 686, 687, 688, 689, 690, and 691, respectively. In some embodiments, the antibody of the antibody drug conjugate comprises a heavy chain variable region comprising the amino acid sequence of SEQ ID NO: 692 and a light chain variable region comprising the amino acid sequence of SEQ ID NO: 693. In some embodiments, the antibody of the antibody drug conjugate is depatuxizumab.

In some embodiments, an antibody-drug conjugate provided herein binds to SLAMF7. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 694, 695, 696, 697, 698, and 699, respectively. In some embodiments, the antibody of the antibody drug conjugate comprises a heavy chain variable region comprising the amino acid sequence of SEQ ID NO: 700 and a light chain variable region comprising the amino acid sequence of SEQ ID NO: 701. In some embodiments, the antibody of the antibody drug conjugate is azintuxizumab.

In some embodiments, an antibody-drug conjugate provided herein binds to SLITRK6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 702, 703, 704, 705, 706, and 707, respectively. In some embodiments, the antibody of the antibody drug conjugate comprises a heavy chain variable region comprising the amino acid sequence of SEQ ID NO: 708 and a light chain variable region comprising the amino acid sequence of SEQ ID NO: 709. In some embodiments, the antibody of the antibody drug conjugate is sirtratumab.

In some embodiments, an antibody-drug conjugate provided herein binds to C4.4a. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 710, 711, 712, 713, 714, and 715, respectively. In some embodiments, the antibody of the antibody drug conjugate comprises a heavy chain variable region comprising the amino acid sequence of SEQ ID NO: 716 and a light chain variable region comprising the amino acid sequence of SEQ ID NO: 717. In some embodiments, the antibody of the antibody drug conjugate is lupartumab.

In some embodiments, an antibody-drug conjugate provided herein binds to GCC. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 718, 719, 720, 721, 722, and 723, respectively. In some embodiments, the antibody of the antibody drug conjugate comprises a heavy chain variable region comprising the amino acid sequence of SEQ ID NO: 724 and a light chain variable region comprising the amino acid sequence of SEQ ID NO: 725. In some embodiments, the antibody of the antibody drug conjugate is indusatumab.

In some embodiments, an antibody-drug conjugate provided herein binds to Axl. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 726, 727, 728, 729, 730, and 731, respectively. In some embodiments, the antibody of the antibody drug conjugate comprises a heavy chain variable region comprising the amino acid sequence of SEQ ID NO: 732 and a light chain variable region comprising the amino acid sequence of SEQ ID NO: 733. In some embodiments, the antibody of the antibody drug conjugate is enapotamab.

In some embodiments, an antibody-drug conjugate provided herein binds to gpNMB. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 734, 735, 736, 737, 738, and 739, respectively. In some embodiments, the antibody of the antibody drug conjugate comprises a heavy chain variable region comprising the amino acid sequence of SEQ ID NO: 740 and a light chain variable region comprising the amino acid sequence of SEQ ID NO: 741. In some embodiments, the anti-gpNMB antibody comprises a heavy chain variable region comprising an amino acid sequence that is at least 80%, at least 85%, at least 90%, at least 95%, at least 98%, or 99% identical to the amino acid sequence of SEQ ID NO: 1179 and a light chain variable region comprising an amino acid sequence that is at least 80% at least 85%, at least 90%, at least 95%, at least 98%, or 99% identical to the amino acid sequence of SEQ ID NO: 1180. In some embodiments, the antibody of the antibody drug conjugate is glembatumumab.

In some embodiments, an antibody-drug conjugate provided herein binds to Prolactin receptor. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 742, 743, 744, 745, 746, and 747, respectively. In some embodiments, the antibody of the antibody drug conjugate comprises a heavy chain variable region comprising the amino acid sequence of SEQ ID NO: 748 and a light chain variable region comprising the amino acid sequence of SEQ ID NO: 749. In some embodiments, the antibody of the antibody drug conjugate is rolinsatamab.

In some embodiments, an antibody-drug conjugate provided herein binds to FGFR2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 750, 751, 752, 753, 754, and 755, respectively. In some embodiments, the antibody of the antibody drug conjugate comprises a heavy chain variable region comprising the amino acid sequence of SEQ ID NO: 756 and a light chain variable region comprising the amino acid sequence of SEQ ID NO: 757. In some embodiments, the antibody of the antibody drug conjugate is aprutumab.

In some embodiments, an antibody-drug conjugate provided herein binds to CDCP1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 758, 759, 760, 761, 762, and 763, respectively. In some embodiments, the antibody of the antibody drug conjugate comprises a heavy chain variable region comprising the amino acid sequence of SEQ ID NO: 764 and a light chain variable region comprising the amino acid sequence of SEQ ID NO: 765. In some embodiments, the antibody of the antibody drug conjugate is Humanized CUB4 #135 HC4-H. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 766, 767, 768, 769, 770, and 771, respectively. In some embodiments, the antibody of the antibody drug conjugate comprises a heavy chain variable region comprising the amino acid sequence of SEQ ID NO: 772 and a light chain variable region comprising the amino acid sequence of SEQ ID NO: 773. In some embodiments, the antibody of the antibody drug conjugate is CUB4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 774, 775, 776, 777, 778, 779, respectively. In some embodiments, the antibody of the antibody drug conjugate comprises a heavy chain variable region comprising the amino acid sequence of SEQ ID NO: 780 and a light chain variable region comprising the amino acid sequence of SEQ ID NO: 781. In some embodiments, the antibody of the antibody drug conjugate is CP13E10-WT. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 782, 783, 784, 785, 786, and 787, respectively. In some embodiments, the antibody of the antibody drug conjugate comprises a heavy chain variable region comprising the amino acid sequence of SEQ ID NO: 788 and a light chain variable region comprising the amino acid sequence of SEQ ID NO: 789. In some embodiments, the antibody of the antibody drug conjugate is CP13E10-54HCv13-89LCv1. In some embodiments, an antibody-drug conjugate provided herein binds to CDCP1. In some embodiments, the antibody of the antibody drug conjugate comprises a heavy chain variable region comprising the amino acid sequence of SEQ ID NO: 764 and a light chain variable region comprising the amino acid sequence of SEQ ID NO: 1215. In some embodiments, the antibody of the antibody drug conjugate is Humanized CUB4 #135 HC4-H. In some embodiments, the antibody of the antibody drug conjugate comprises a heavy chain variable region comprising the amino acid sequence of SEQ ID NO: 772 and a light chain variable region comprising the amino acid sequence of SEQ ID NO: 1216. In some embodiments, the antibody of the antibody drug conjugate is CUB4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 774, 775, 1217, 777, 778, 779, respectively. In some embodiments, the antibody of the antibody drug conjugate comprises CDR-H3, comprising the amino acid sequence of SEQ ID NO: 1209.

In some embodiments, an antibody-drug conjugate provided herein binds to ASCT2. In some embodiments, the antibody of the antibody drug conjugate comprises a heavy chain variable region comprising the amino acid sequence of SEQ ID NO: 790 and a light chain variable region comprising the amino acid sequence of SEQ ID NO: 791. In some embodiments, the antibody of the antibody drug conjugate is KM8094a. In some embodiments, the antibody of the antibody drug conjugate comprises a heavy chain variable region comprising the amino acid sequence of SEQ ID NO: 792 and a light chain variable region comprising the amino acid sequence of SEQ ID NO: 793. In some embodiments, the antibody of the antibody drug conjugate is KM8094b. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 794, 795, 796, 797, 798, and 799, respectively. In some embodiments, the antibody of the antibody drug conjugate comprises a heavy chain variable region comprising the amino acid sequence of SEQ ID NO: 800 and a light chain variable region comprising the amino acid sequence of SEQ ID NO: 801. In some embodiments, the antibody of the antibody drug conjugate is KM4018.

In some embodiments, an antibody-drug conjugate provided herein binds to CD123. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 802, 803, 804, 805, 806, and 807, respectively. In some embodiments, the antibody of the antibody drug conjugate comprises a heavy chain variable region comprising the amino acid sequence of SEQ ID NO: 808 and a light chain variable region comprising the amino acid sequence of SEQ ID NO: 809. In some embodiments, the antibody of the antibody drug conjugate is h7G3. See WO 2016201065.

In some embodiments, an antibody-drug conjugate provided herein binds to GPC3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 810, 811, 812, 813, 814, and 815, respectively. In some embodiments, the antibody of the antibody drug conjugate comprises a heavy chain variable region comprising the amino acid sequence of SEQ ID NO: 816 and a light chain variable region comprising the amino acid sequence of SEQ ID NO: 817. In some embodiments, the antibody of the antibody drug conjugate is hGPC3-1. See WO 2019161174. In some embodiments, an antibody-drug conjugate provided herein binds to GPC3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 810, 1218, 812, 1219, 814, and 815, respectively. In some embodiments, the antibody of the antibody drug conjugate comprises CDR-H2, comprising the amino acid sequence of SEQ ID NO: 1218. In some embodiments, the antibody of the antibody drug conjugate comprises CDR-L1, comprising the amino acid sequence of SEQ ID NO: 1219.

In some embodiments, an antibody-drug conjugate provided herein binds to B6A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 818, 819, 820, 821, 822, and 823, respectively. In some embodiments, the antibody of the antibody drug conjugate comprises a heavy chain variable region comprising the amino acid sequence of SEQ ID NO: 824 and a light chain variable region comprising the amino acid sequence of SEQ ID NO: 825. In some embodiments, the antibody of the antibody drug conjugate is h2A2. See PCT/US20/63390. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 826, 827, 828, 829, 830, and 831, respectively. In some embodiments, the antibody of the antibody drug conjugate comprises a heavy chain variable region comprising the amino acid sequence of SEQ ID NO: 832 and a light chain variable region comprising the amino acid sequence of SEQ ID NO: 833. In some embodiments, the antibody of the antibody drug conjugate is h15H3. See WO 2013/123152.

In some embodiments, an antibody-drug conjugate provided herein binds to PD-L1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 834, 835, 836, 837, 838, and 839, respectively. In some embodiments, the antibody of the antibody drug conjugate comprises a heavy chain variable region comprising the amino acid sequence of SEQ ID NO: 840 and a light chain variable region comprising the amino acid sequence of SEQ ID NO: 841. In some embodiments, the antibody of the antibody drug conjugate is SG-559-01. See PCT/US2020/054037.

In some embodiments, an antibody-drug conjugate provided herein binds to TIGIT. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 842, 843, 844, 845, 846, and 847, respectively. In some embodiments, the antibody of the antibody drug conjugate comprises a heavy chain variable region comprising the amino acid sequence of SEQ ID NO: 848 and a light chain variable region comprising the amino acid sequence of SEQ ID NO: 849. In some embodiments, the antibody of the antibody drug conjugate is Clone 13 (also known as ADI-23674 or mAb13). See WO 2020041541. In some embodiments, an antibody-drug conjugate provided herein binds to TIGIT. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 842, 843, 1220, 845, 846, and 847, respectively. In some embodiments, the antibody of the antibody drug conjugate comprises CDR-H3, comprising the amino acid sequence of SEQ ID NO: 1220.

In some embodiments, an antibody-drug conjugate provided herein binds to STN. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 850, 851, 852, 853, 854, and 855, respectively. In some embodiments, the antibody of the antibody drug conjugate comprises a heavy chain variable region comprising the amino acid sequence of SEQ ID NO: 856 and a light chain variable region comprising the amino acid sequence of SEQ ID NO: 857. In some embodiments, the antibody of the antibody drug conjugate is 2G12-2B2. See WO 2017083582.

In some embodiments, an antibody-drug conjugate provided herein binds to CD33. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 858, 859, 860, 861, 862, and 863, respectively. In some embodiments, the antibody of the antibody drug conjugate comprises a heavy chain variable region comprising the amino acid sequence of SEQ ID NO: 864 and a light chain variable region comprising the amino acid sequence of SEQ ID NO: 865. In some embodiments, the antibody of the antibody drug conjugate comprises a heavy chain variable region comprising the amino acid sequence of SEQ ID NO: 864 and a light chain variable region comprising the amino acid sequence of SEQ ID NO: 1221. In some embodiments, the antibody of the antibody drug conjugate is h2H12. See WO2013173496.

In some embodiments, an antibody-drug conjugate provided herein binds to NTBA (also known as CD352). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 866, 867, 868, 869, 870, and 871, respectively. In some embodiments, the antibody of the antibody drug conjugate comprises a heavy chain variable region comprising the amino acid sequence of SEQ ID NO: 872 and a light chain variable region comprising the amino acid sequence of SEQ ID NO: 873. In some embodiments, the antibody of the antibody drug conjugate is h20F3 HDLD. See WO 2017004330.

In some embodiments, an antibody-drug conjugate provided herein binds to BCMA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 874, 875, 876, 877, 878, and 879, respectively. In some embodiments, the antibody of the antibody drug conjugate comprises a heavy chain variable region comprising the amino acid sequence of SEQ ID NO: 880 and a light chain variable region comprising the amino acid sequence of SEQ ID NO: 881. In some embodiments, the antibody of the antibody drug conjugate is SEA-BCMA (also known as hSG16.17). See WO 2017/143069.

In some embodiments, an antibody-drug conjugate provided herein binds to Tissue Factor (also known as TF). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 882, 883, 884, 885, 886, and 887, respectively. In some embodiments, the antibody of the antibody drug conjugate comprises a heavy chain variable region comprising the amino acid sequence of SEQ ID NO: 888 and a light chain variable region comprising the amino acid sequence of SEQ ID NO: 889. In some embodiments, the antibody of the antibody drug conjugate is tisotumab. See WO 2010/066803 and U.S. Pat. No. 9,150,658.

### Camptothecin Compounds:

The Camptothecin compounds utilized in the various embodiments described herein are represented by the formula:

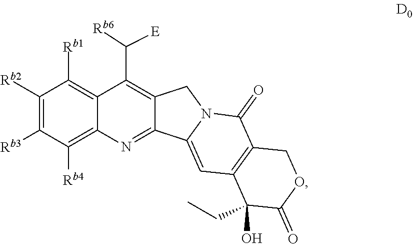

or a salt thereof; wherein;
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₆ alkyl-O—C₁-C₆ alkyl-, C₁-C₈ aminoalkyl, (C₁-C₄ alkylamino)-C₁-C₈ alkyl-, N,N—(C₁-C₄ hydroxyalkyl) C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, heteroaryl-C₁-C₆ hydroxyalkyl, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆ alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—; or

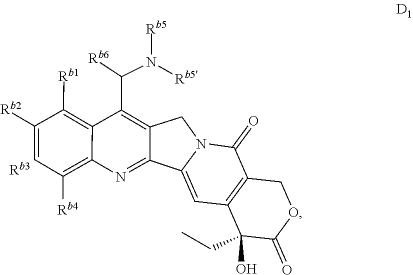

or a salt thereof; wherein;
R^{b1} is selected from the group consisting of H, halogen, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃ C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a} are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—.

Still other Camptothecin compounds useful in the context of the Camptothecin Conjugates and Camptothecin Linker compounds described herein are Camptothecin compounds of formula D₁ D₁ₐ, D_{1b}, or any subformula thereof, or any of the compounds of Table I, which in some embodiments have an additional group including, but not limited to a hydroxyl, thiol, amine or amide functional group whose oxygen, sulfur or optionally substituted nitrogen atom is capable of incorporation into a linker, and is capable of being released from a Camptothecin Conjugate as a free drug. In some embodiments, that functional group provides the only site on the camptothecin compound available for attachment to the Linker Unit (Q). The resulting drug-linker moiety of a Camptothecin Conjugate is one that is capable of releasing active free drug at the site targeted by its Ligand Unit in order to exert a cytotoxic, cytostatic or immunosuppressive effect.

“Free drug” refers to drug, as it exists once released from the drug-linker moiety. In some embodiments, the free drug includes a fragment of the Releasable Linker or Spacer Unit (Y) group. Free drug, which includes a fragment of the Releasable Linker or Spacer Unit (Y), are released from the remainder of the drug-linker moiety via cleavage of the releasable linker or released via the cleavage of a bond in the Spacer Unit (Y) group and is biologically active after release. In some embodiments, the free drug differs from the conjugated drug in that the functional group of the free drug for attachment to the self-immolative assembly unit is no longer associated with components of the Camptothecin Conjugate (other than a previously shared heteroatom). For example, the free hydroxyl functional group of an alcohol-containing drug can be represented as D-O *H, whereas in the conjugated form the oxygen heteroatom designated by O* is incorporated into the methylene carbamate unit of a self-immolative unit. Upon activation of the self-immolative moiety and release of free drug, the covalent bond to O* is replaced by a hydrogen atom so that the oxygen heteroatom designated by O* is present on the free drug as —O—H.

### Linker Unit (Q)

As noted above, is some embodiments, the Linker Unit Q has a formula selected from the group consisting of:
Z-A-RL-; -Z-A-RL-Y-; -Z-A-S*-RL-; -Z-A-B(S*)-RL-; -Z-A-S*-RL-Y-; and -Z-A-B(S*)-RL-Y-;
   wherein Z is a Stretcher Unit; A is a bond or a Connector Unit; B is a Parallel Connector Unit; S* is a Partitioning Agent; RL is Releasable Linker, and Y is a Spacer Unit; and wherein the point of attachment of D to Q is through any one of the heteroatoms of the hydroxyl and primary and secondary amines present on formula D₁ D₁, Dib, or any subformula thereof, or any of the compounds of Table I.

In other embodiments, the Linker Unit Q has a formula selected from the group consisting of:
-Z-A-; -Z-A-RL-; -Z-A-S*-W-; -Z-A-B(S*)-W-; -Z-A-S*-RL-; -Z-A-B(S*)-RL-; -Z-A-S* -W-RL-; and -Z-A-B(S*)-W-RL-;
   wherein Z is a Stretcher Unit, A is a bond or a Connector Unit; B is a Parallel Connector Unit; S* is a Partitioning Agent; RL is a Releasable Linker other than a Glycoside (e.g., Glucuronide) Unit; and W is an Amino Acid Unit; and
   wherein the point of attachment to Q is through the hydroxyl group substituent of the lactone ring of formula D₁ D₁, Dib, or any subformula thereof, or any of the compounds of Table I.

In one group of embodiments, Q has a formula selected from the group consisting of: -Z-A-5*-RL- and -Z-A-5*-RL-Y-.

In another group of embodiments, Q has a formula selected from the group consisting of -Z-A-B(S*)-RL- and -Z-A-B(S*)-RL-Y-.

In still another group of embodiments, Q has a formula selected from the group consisting of -Z-A-RL- and -Z-A-RL-Y-.

### Stretcher Unit (Z) or (Z′):

A Stretcher Unit (Z) is a component of a Camptothecin Conjugate or a Camptothecin-Linker Compound or other intermediate that acts to connect the Ligand Unit to the remainder of the conjugate. In that regard a Stretcher Unit, prior to attachment to a Ligand Unit (i.e. a Stretcher Unit precursor, Z′), has a functional group that can form a bond with a functional group of a targeting ligand.

In some embodiments, a Stretcher Unit precursor (Z′) has an electrophilic group that is capable of interacting with a reactive nucleophilic group present on a Ligand Unit (e.g., an antibody) to provide a covalent bond between a Ligand Unit and the Stretcher Unit of a Linker Unit. Nucleophilic groups on an antibody having that capability include but are not limited to, sulfhydryl, hydroxyl and amino functional groups. The heteroatom of the nucleophilic group of an antibody can be reactive to an electrophilic group on a Stretcher Unit precursor and can provide a covalent bond between the Ligand Unit and Stretcher Unit of a Linker Unit or Drug-Linker moiety. Useful electrophilic groups for that purpose include, but are not limited to, maleimide, haloacetamide groups, and NHS esters. The electrophilic group provides a convenient site for antibody attachment to form a Camptothecin Conjugate or Ligand Unit-Linker intermediate.

In other embodiments, a Stretcher Unit precursor has a reactive site which has a nucleophilic group that is reactive to an electrophilic group present on a Ligand Unit (e.g., an antibody). Useful electrophilic groups on an antibody for that purpose include, but are not limited to, aldehyde and ketone carbonyl groups. The heteroatom of a nucleophilic group of a Stretcher Unit precursor can react with an electrophilic group on an antibody and form a covalent bond to the antibody. Useful nucleophilic groups on a Stretcher Unit precursor for that purpose include, but are not limited to, hydrazide, hydroxylamine, amino, hydrazine, thiosemicarbazone, hydrazine carboxylate, and arylhydrazide. The electrophilic group on an antibody provides a convenient site for antibody attachment to form a Camptothecin Conjugate or Ligand Unit-Linker intermediate.

In some embodiments, a sulfur atom of a Ligand Unit is bound to a succinimide ring system of a Stretcher Unit formed by reaction of a thiol functional group of a targeting ligand with a maleimide moiety of the corresponding Stretcher Unit precursor. In other embodiments, a thiol functional group of a Ligand Unit reacts with an alpha haloacetamide moiety to provide a sulfur-bonded Stretcher Unit by nucleophilic displacement of its halogen substituent.

Representative Stretcher Units of such embodiments include those having the structures of:

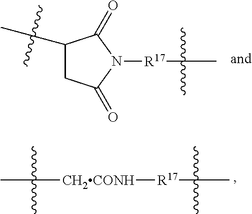

wherein the wavy line adjacent to R¹⁷ indicates attachment to the Parallel Connector Unit (B) or Connector Unit (A) if B is absent, or a Partitioning Agent (S*), if B is absent, the other wavy line indicates covalent attachment to a sulfur atom of a Ligand Unit, and R¹⁷ is -C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, -C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, -C₁-C₁₀ alkylene-NH—, —C₁-C₁₀ heteroalkylene-NH—, —C₃-C₈ carbocyclo-NH—, —O—(C₁-C₈ alkylene)—NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)—NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)—NH—, —(C₃-C₈ heterocyclo)-C₁-C₁₀ alkylene-NH—, —C₁-C₁₀ alkylene-S-, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S-, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S-, —C₁-C₁₀alkylene-(C₃-C₈ heterocyclo)-S—, or —(C₃-C₈ heterocyclo)-C₁-C₁₀ alkylene-S—.

Representative Stretcher Units of such embodiments include those having the structures of:

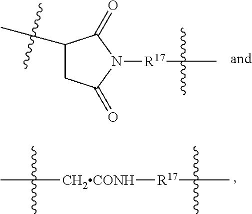

wherein the wavy line adjacent to R¹⁷ indicates attachment to the Parallel Connector Unit (B) or Connector Unit (A) if B is absent, or a Partitioning Agent (S*), if B is absent, the other wavy line indicates covalent attachment to a sulfur atom of a Ligand Unit, and R¹⁷ is —C₁-C₁₀ alkylene-, —CH₂—CH₂—(OCH₂CH₂)ₖ—, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₂₀ alkylene-, —C₁-C₂₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₂₀ alkylene-arylene-C(═O)—, -arylene-C₁-C₂₀ alkylene-C(═O)—, —C₁-C₂₀ alkylene-(C₃-C₈ carbocyclo)-C(═O)—, —(C₃-C₈ carbocyclo)-C₁-C₂₀ alkylene-C(═O)—, —C₃-C₈ heterocyclo-C(═O)—, —C₁-C₁₀alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, C₃-C₈ carbocyclo-NH—, —O—(C₁-C₈ alkylene)—NH—, -arylene-NH—, —C₁-C₂₀ alkylene-arylene-NH—, -arylene-C₁-C₂₀ alkylene-NH—, —C₁-C₂₀ alkylene-(C₃-C₈ carbocyclo)—NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)—NH—, —(C₃-C₈ heterocyclo)-C₁-C₂₀ alkylene-NH—, —C₁-C₁₀ alkylene-S-, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₂₀ alkylene-S—, —C₁-C₂₀ alkylene-(C₃-C₈ carbocyclo)-S—, (C₃-C₈ carbocyclo)-C₁-C₂₀ alkylene-S—, —C₃-C₈ heterocyclo-S—, —C₁-C₁₀alkylene-(C₃-C₈ heterocyclo)-S—, or —(C₃-C₈ heterocyclo)-C₁-C₂₀ alkylene-S—, wherein k is an integer ranging from 1 to 36. In some embodiments, R¹⁷ is -C₁-C₂₀ alkylene-. In some embodiments, R¹⁷ is —CH₂—CH₂—(OCH₂CH₂)ₖ—, wherein k is an integer ranging from 1 to 36.

In some embodiments, the R¹⁷ group is optionally substituted by a Basic Unit (BU) such as an aminoalkyl moiety, e.g. —(CH₂)_{X}NH₂, —(CH₂)_{X}NHR^{a}, and —(CH₂)_{X}NR^{a}₂, wherein subscript x is an integer of from 1-4 and each R^{a} is independently selected from the group consisting of C₁₄ alkyl and C₁₄ haloalkyl, or two R^{a} groups are combined with the nitrogen to which they are attached to form an azetidinyl, pyrrolidinyl or piperidinyl group.

An illustrative Stretcher Unit is that of Formula Za or Za-BU in which R¹⁷ is —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₂₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₂₀ alkylene-(C₃-C₈ heterocyclo)-C(═O)—, or —(C₃-C₈ heterocyclo)-C₁-C₁₀ alkylene-C(═O)—.

Accordingly, some preferred embodiments are represented by formula Za and Za-BU:

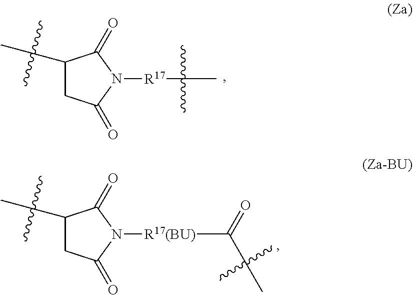

wherein the wavy line adjacent the carbonyl carbon atom indicates attachment to L^{P}, B, A, or S*, in the formulae above, depending on the presence or absence of A and/or B, and the other wavy line indicates covalent bonding of the succinimide ring carbon atom to a sulfur atom of a Ligand Unit. During synthesis, the basic amino functional group of the Basic Unit (BU) can be protected by a protecting group.

More preferred embodiments of Stretcher Units of formula Za and Za-BU are as follows:

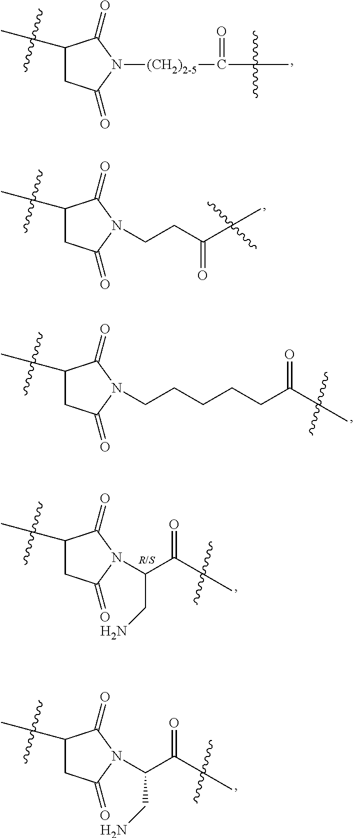

wherein the wavy line adjacent the carbonyl carbon atom indicates attachment to B, A, or S*, in the formulae above, depending on the presence or absence of A and/or B, and the other wavy line indicates covalent bonding of the succinimide ring carbon atom to a sulfur atom of a Ligand Unit.

Other preferred embodiments of Stretcher Units of formula Za and Za-BU are as follows:

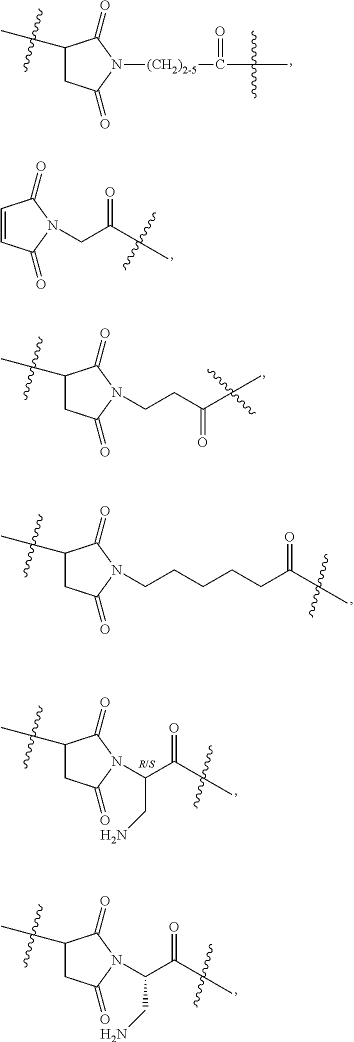

wherein the wavy line adjacent the carbonyl carbon atom indicates attachment to B, A, or S*, in the formulae above, depending on the presence or absence of A and/or B, and the other wavy line indicates covalent bonding of the succinimide ring carbon atom to a sulfur atom of a Ligand Unit.

It will be understood that a Ligand Unit-substituted succinimide may exist in hydrolyzed form(s). Those forms are exemplified below for hydrolysis of Za or Za-BU, wherein the structures representing the regioisomers from that hydrolysis have formula Zb and Zc or Zb-BU and Zc-BU.

Accordingly, in other preferred embodiments a Stretcher unit (Z) is comprised of a succinic acid-amide moiety represented by the following:

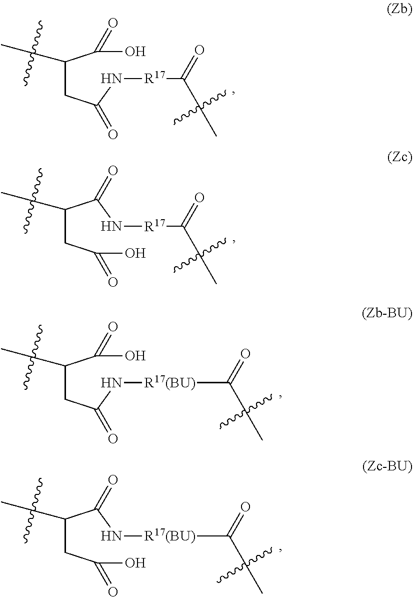

wherein the wavy line adjacent to the carbonyl carbon atom bonded to R¹⁷ and the wavy line adjacent to the carbon atom of the acid-amide moiety is as defined for Za or Za-BU, depending on the presence or absence of A and/or B; and R¹⁷ is -C₁-C₈ alkylene-, wherein in Zb-BU and Zc-BU the alkylene is substituted by a Basic Unit (BU), wherein BU is —(CH₂ )_{X}NH₂, —(CH₂)_{X}NHR^{a}, or —(CH₂)ₓN(R^{a})₂, wherein subscript x is an integer of from 1-4 and each R^{a} is independently selected from the group consisting of C₁₄ alkyl and C₁-6 haloalkyl, or both R^{a} together with the nitrogen to which they are attached define an azetidinyl, pyrrolidinyl or piperidinyl group.

In more preferred embodiment, -Z-A- comprises a moiety derived from a maleimido-alkanoic acid moiety or an mDPR moiety. See, for example, see WO 2013/173337. In one group of embodiments, Z-A- is derived from a maleimido-propionyl moiety.

Accordingly, in some of those more preferred embodiments, a Stretcher unit (Z) is comprised of a succinic acid-amide moiety represented by the structure of formula Zb′, Zc′, (R/S)-Zb′-BU, (S)-Zb′-BU, (R/S)-Zc′-BU or (S)-Zc′-BU as follows:

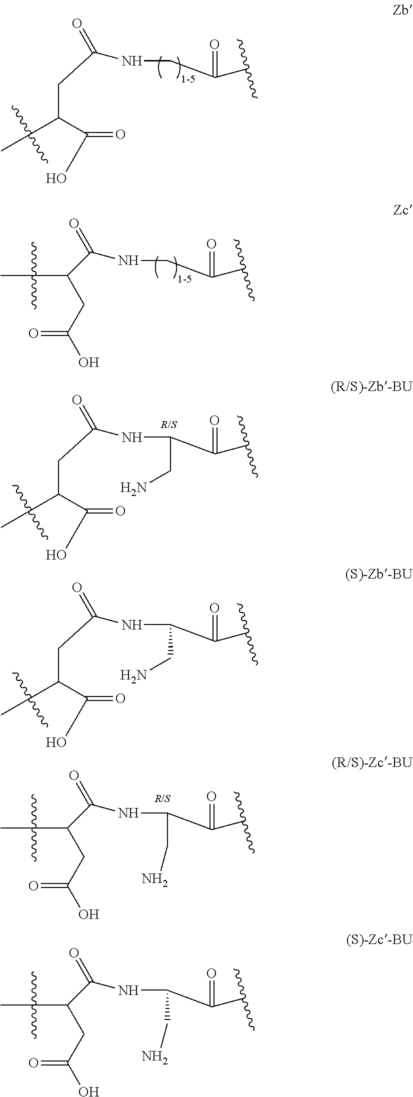

wherein the wavy lines are as defined for Za or Za-BU.

In particularly preferred embodiments, a Stretcher unit (Z) is comprised of a succinimide moiety represented by the structure of

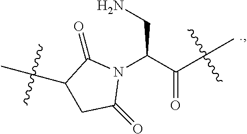 [SMILES: *C(=O)[C@H](CN)N1C(=O)CC(*)C1=O]

which may be generated from a maleimido-amino-propionyl (mDPR) analog (a 3-amino-2-(2,5-dioxo-2,5-dihydro-1 H-pyrrol-1-yl)propanoic acid derivative), or is comprised of a succinic acid-amide moiety represented by the structure of:

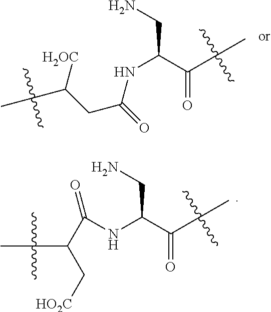

Illustrative Stretcher Units bonded to a Connector Unit (A) which are comprised of Za′, Zb′ or Zc′, in which —R¹⁷— of Za, Zb or Zc is —CH₂— or —CH₂CH₂—, or are comprised of Za′-BU, Zb′-BU or Zc′-BU in which —R1BU)— of Za-BU, Zb-BU or Zc-BU is —CH(CH₂NH₂)—, have the following structures:

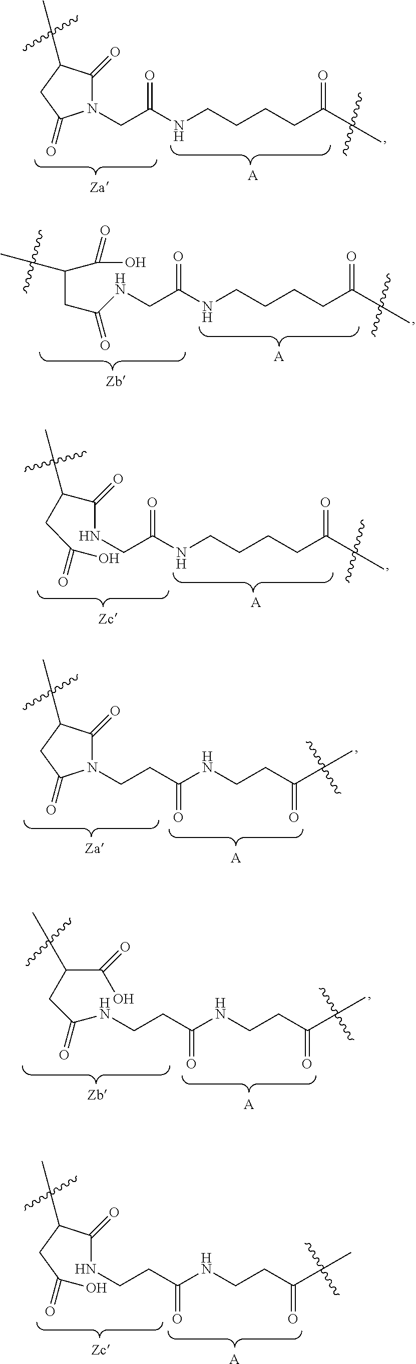

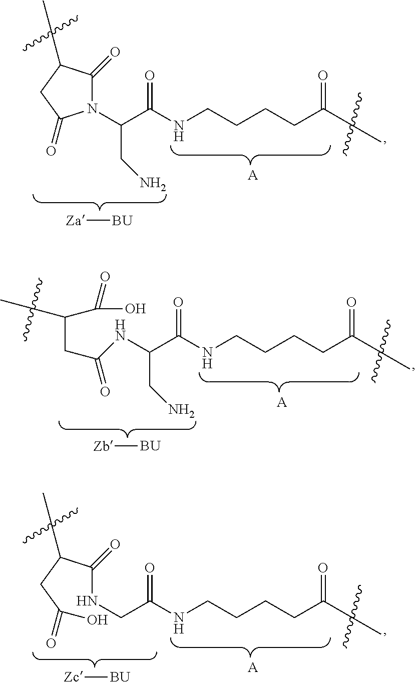

wherein the wavy lines are as defined for Za or Za-BU.

Other Stretcher Units bonded to a Ligand Unit (L) and a Connector Unit (A) have the structures above wherein A in any one of the above -Za-A-, -Za(BU)-A-, -Za′-A-, -Za′(BU)-A-, -Zb-A-, -Zb(BU)-A-, -Zb′-A-, -Zb′(BU)-, -Zc‘-A- and Zc’(BU)-A- structures is replaced by a Parallel Connector Unit having the structure of:

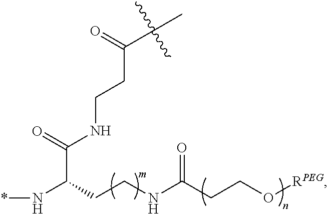

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to a Stretcher Unit corresponding in structure to formula Za, Za′, Zb′ or Zc′ and the wavy line indicates covalent attachment to the Releasable Linker (RL).

Illustrative Stretcher Units prior to conjugation to the Ligand Unit (i.e., Stretcher Unit precursors) are comprised of a maleimide moiety and are represented by structures including that of formula Z′a

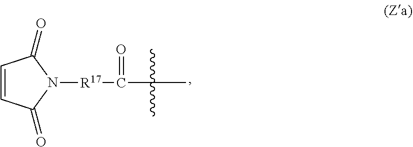

wherein the wavy line adjacent the carbonyl carbon atom indicates attachment to B, A, or S*, in the formulae above, depending on the presence or absence of A and/or B, R¹⁷ is —CH₂)₁₋₅ —, optionally substituted with a Basic Unit, such as an optionally substituted aminoalkyl, e.g., —(CH₂)_{X}NH₂, —(CH₂)_{X}NHR^{a}, and —(CH₂)_{X}N(R^{a}) 2, wherein subscript x is an integer of from 1-4 and each IV is independently selected from the group consisting of C₁₄ alkyl and C₁-6 haloalkyl, or two IV groups are combined with the nitrogen to which they are attached to form an azetidinyl, pyrrolidinyl or piperidinyl group.

Other illustrative Stretcher Units prior to conjugation to the Ligand Unit (i.e., Stretcher Unit precursors) are comprised of a maleimide moiety and are represented by structures including that of formula Z′a-BU.

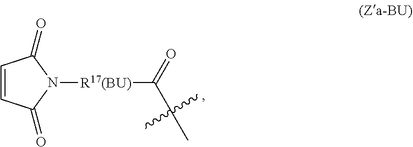

wherein the wavy line adjacent the carbonyl carbon atom indicates attachment to B, A, or S*, in the formulae above, depending on the presence or absence of A and/or B, R¹⁷ is —CH₂)₁₋₅-, substituted with a Basic Unit, such as an optionally substituted aminoalkyl, e.g., —(CH₂ )_{X}NH₂, —(CH₂)_{X}NHR^{a}, and —(CH₂)_{X}N(R^{a})₂, wherein subscript x is an integer of from 1-4, preferably R¹⁷ is —CH₂— or —CH₂CH₂— and subscript x is 1 or 2, and each IV is independently selected from the group consisting of C₁-6 alkyl and C₁-6 haloalkyl, or two IV groups are combined with the nitrogen to which they are attached to form an azetidinyl, pyrrolidinyl or piperidinyl group.

In some preferred embodiments of formula Z′a, a Stretcher Unit precursor (Z′) is represented by one of the following structures:

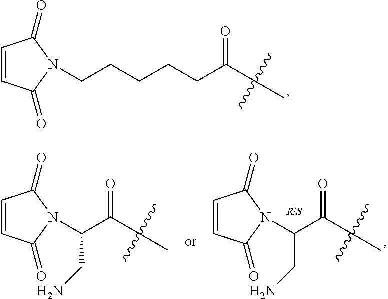

wherein the wavy line adjacent to the carbonyl is as defined for Z′a or Z′a-BU.

In more preferred embodiments the Stretcher unit precursor (Z′) is comprised of a maleimide moiety and is represented by the structure of:

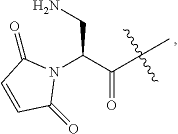 [SMILES: *C(=O)[C@H](CN)N1C(=O)C=CC1=O]

wherein the wavy line adjacent to the carbonyl is as defined for Za′ and the amino group is optional protonated or protected by an amino protecting group.

In Stretcher Units having a BU moiety, it will be understood that the amino functional group of that moiety is typically protected by an amino protecting group during synthesis, e.g., an acid labile protecting group (e.g., BOC).

Illustrative Stretcher Unit precursors covalently attached to a Connector Unit that are comprised of the structure of Z′a or Z′a-BU in which —R¹⁷— or —R¹⁷(BU)— is —CH₂—, —CH₂CH₂— or —CH(CH₂NH₂)— have the following structures:

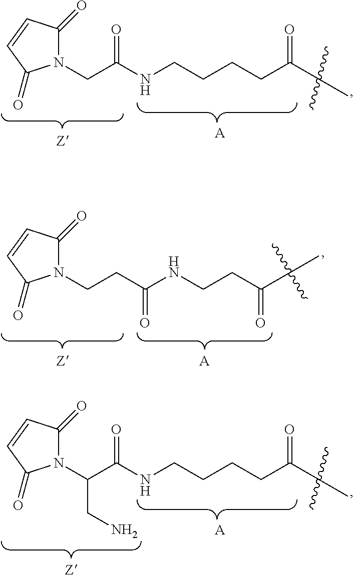

wherein the wavy line adjacent to the carbonyl is as defined for Z′a or Z′a-BU.

Other Stretcher Unit precursors bonded a Connector Unit (A) have the structures above wherein A in any one of the above Z′-A- and Z′(BU)-A- structures is replaced by a Parallel Connector Unit and Partitioning Agent (-B(S*)-) having the structure of

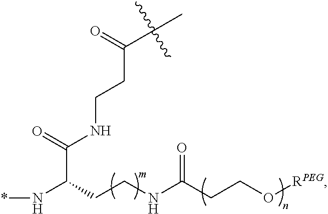

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to the Stretcher Unit precursor corresponding in structure to formula Za or Za′ and the wavy line indicates covalent attachment to RL. In instances such as those shown here, the shown PEG group is meant to be exemplary of a variety of Partitioning Agents including PEG groups of different lengths and other Partitioning Agents that can be directly attached or modified for attachment to the Parallel Connector Unit.

In another embodiment, the Stretcher Unit is attached to the Ligand Unit via a disulfide bond between a sulfur atom of the Ligand Unit and a sulfur atom of the Stretcher unit. A representative Stretcher Unit of this embodiment is depicted within the square brackets of Formula Zb:

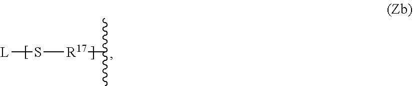

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀alkylene-(C₃-C₈ heterocyclo)-C(═O)—, —(C₃-C₈ heterocyclo)-C₁-C₁₀alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, -C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀alkylene-(C₃-C₈ heterocyclo)-S—, or —(C₃-C₈ heterocyclo)-C₁-C₁₀ alkylene-S—.

In yet another embodiment, the reactive group of a Stretcher Unit precursor contains a reactive site that can form a bond with a primary or secondary amino group of a Ligand Unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher Units of this embodiment are depicted within the square brackets of Formulas Zci, Zcii and Zciii:

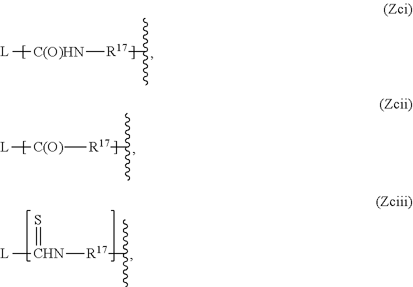

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀alkylene-(C₃-C₈ heterocyclo)-C(═O)—, —(C₃-C₈ heterocyclo)-C₁-C₁₀alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, -C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀alkylene-(C₃-C₈ heterocyclo)-S—, or —(C₃-C₈ heterocyclo)-C₁-C₁₀ alkylene-S—.

In still other embodiments, the reactive group of the Stretcher Unit precursor contains a reactive nucleophile that is capable of reacting with an electrophile present on, or introduced to, a Ligand Unit. For example, a carbohydrate moiety on a targeting ligand can be mildly oxidized using a reagent such as sodium periodate and the resulting electrophilic functional group (—CHO) of the oxidized carbohydrate can be condensed with a Stretcher Unit precursor that contains a reactive nucleophile such as a hydrazide, an oxime, a primary or secondary amine, a hydrazine, a thiosemicarbazone, a hydrazine carboxylate, or an arylhydrazide such as those described by Kaneko, T. et al. (1991) Bioconjugate Chem. 2:133-41. Representative Stretcher Units of this embodiment are depicted within the square brackets of Formulas Zdi, Zdii, and Zdiii:

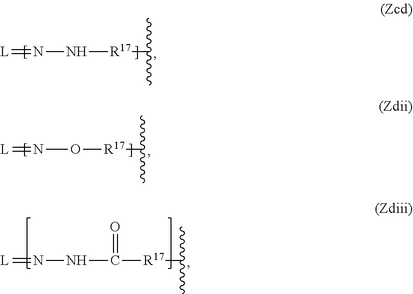

wherein the wavy line indicates attachment to the Parallel Connector Unit (B) or Connector Unit (A), or a Partitioning Agent (S*), if A and B are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀alkylene-(C₃-C₈ heterocyclo)-C(═O)—, —(C₃-C₈ heterocyclo)-C₁-C₁₀alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁⁻C₈ alkylene)-S—, -arylene-S—, —C₁-C₁₀ alkylene-arylene-S—, -arylene-C₁-C₁₀ alkylene-S—, -C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀alkylene-(C₃-C₈ heterocyclo)-S—, or —(C₃-C₈ heterocyclo)-C₁-C₁₀ alkylene-S—.

In some aspects of the prevent invention the Stretcher Unit has a mass of no more than about 1000 daltons, no more than about 500 daltons, no more than about 200 daltons, from about 30, 50, or 100 daltons to about 1000 daltons, from about 30, 50, or 100 daltons to about 500 daltons, or from about 30, 50, or 100 daltons to about 200 daltons.

### Connector Unit (A)

In some embodiments, a Connector Unit (A), is included in a Camptothecin Conjugate or Camptothecin-Linker Compound in instances where it is desirable to add additional distance between the Stretcher Unit (Z) or precursor thereof (Z′) and the Releasable Linker. In some embodiments, the extra distance will aid with activation within RL. Accordingly, the Connector Unit (A), when present, extends the framework of the Linker Unit. In that regard, a Connector Unit (A) is covalently bonded with the Stretcher Unit (or its precursor) at one terminus and is covalently bonded to the optional Parallel Connector Unit or the Partitioning Agent (S*) at its other terminus.

The skilled artisan will appreciate that the Connector Unit can be any group that serves to provide for attachment of the Releasable Linker to the remainder of the Linker Unit (Q). The Connector Unit can be, for example, comprised of one or more (e.g., 1-10, preferably, 1, 2, 3, or 4) proteinogenic or non-proteinogenic amino acid, amino alcohol, amino aldehyde, diamino residues. In some embodiments, the Connector Unit is a single proteinogenic or non-proteinogenic amino acid, amino alcohol, amino aldehyde, or diamino residue. An exemplary amino acid capable of acting as Connector units is β-alanine.

In some of those embodiments, the Connector Unit has the formula denoted below:

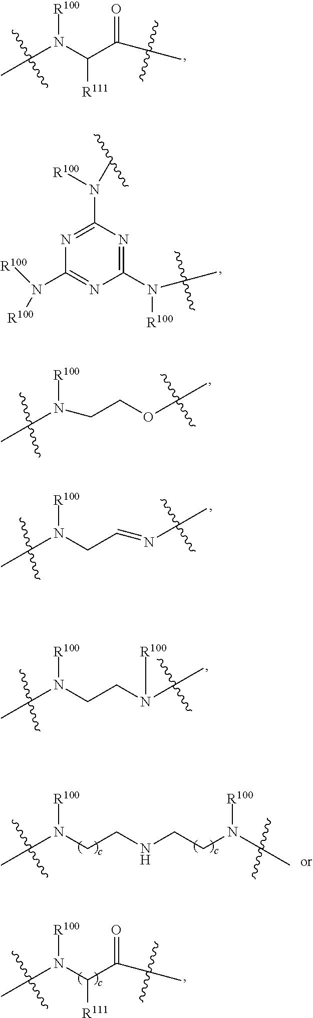

wherein the wavy lines indicate attachment of the Connector Unit within the Camptothecin Conjugate or Camptothecin Linker Compound; and wherein R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

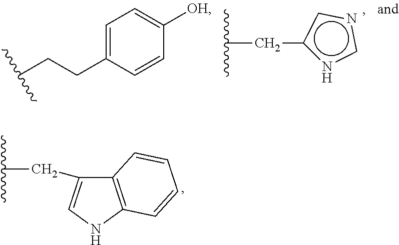

and each R¹⁰⁰ is independently selected from hydrogen or —C₁-C₃ alkyl, preferably hydrogen or CH₃; and subscript c is an independently selected integer from 1 to 10, preferably 1 to 3.

A representative Connector Unit having a carbonyl group for attachment to the Partitioning Agent (S*) or to -B(S*)- is as follows:

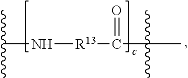

wherein in each instance R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈ carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and the subscript c is an integer ranging from 1 to 4. In some embodiments R¹³ is —C₁-C₆ alkylene and c is 1.

Another representative Connector Unit having a carbonyl group for attachment to Partitioning Agent (S*) or to -B(S*)- is as follows:

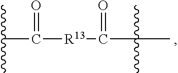

wherein R¹³ is —C₁-C₆ alkylene-, —C₃-C₈carbocyclo-, -arylene-, —C₁-C₁₀heteroalkylene-, —C₃-C₈heterocyclo-, —C₁-C₁₀ alkylene-arylene-, -arylene-C₁-C₁₀ alkylene-, —C₁-C₁₀ alkylene-(C₃⁻C₈carbocyclo)-, —(C₃-C₈carbocyclo)-C₁-C₁₀alkylene-, —C₁-C₁₀alkylene-(C₃-C₈ heterocyclo)-, or —(C₃-C₈ heterocyclo)-C₁-C₁₀ alkylene-. In some embodiments R¹³ is —C₁-C₆ alkylene.

A representative Connector Unit having a NH moiety that attaches to Partitioning Agent (S*) or to -B(S*)- is as follows:

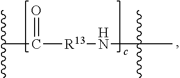

wherein in each instance, R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈ carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and subscript c is from 1 to 14. In some embodiments R¹³ is C₁-C₆ alkylene and subscript c is 1.

Another representative Connector Unit having a NH moiety that attaches to Partitioning Agent (S*) or to —B(S*)— is as follows:

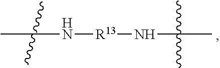

wherein R¹³ is —C₁-C₆ alkylene-, —C₃-C₈carbocyclo-, -arylene-, —C₁-C₁₀heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, (C₃-C₈ heterocyclo)-C₁-C₁₀ alkylene-, —C(═O)C₁-C₆ alkylene- or —C₁-C₆ alkylene-C(═O)—C₁-C₆ alkylene.

Selected embodiments of Connector Units include those having the following structure of:

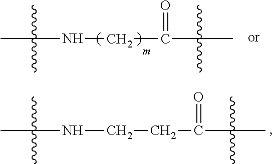 [SMILES: *NCC(*)=O ; *NCCC(*)=O]

wherein the wavy line adjacent to the nitrogen indicates covalent attachment a Stretcher Unit (Z) (or its precursor Z′), and the wavy line adjacent to the carbonyl indicates covalent attachment to Partitioning Agent (S*) or to —B(S*)—; and m is an integer ranging from 1 to 6, preferably 2 to 6, more preferably 2 to 4.

### Releasable Linker (RL)

The Releasable Linker (RL) is capable of linking to the Spacer Unit (Y) or the Drug Unit (D). RL comprises a cleavable bond (i.e., a reactive site) that upon action by an enzyme present within a hyper-proliferating cell or hyper-activated immune cells or characteristic of the immediate environment of these abnormal or unwanted cells, or upon non-enzymatic action due to conditions more likely experienced by hyper-proliferating cells in comparison to normal cells, releases free drug. Alternatively, RL comprises a cleavable bond that is more likely acted upon intracellularly in a hyper-proliferating cell or hyper-activated immune cell due to preferential entry into such cells in comparison to normal cells.

### Peptide Releasable Linkers

In some embodiments, the Releasable Linker is a Peptide Releasable Linker. In some embodiments, the Peptide Releasable Linker (RL) will comprise one or more contiguous or non-contiguous sequences of amino acids (e.g., so that RL has 1 to no more than 12 amino acids). The Peptide Releasable Linker can comprise or consist of, for example, an amino acid, a dipeptide, tripeptide, tetrapeptide, pentapeptide, hexapeptide, heptapeptide, octapeptide, nonapeptide, decapeptide, undecapeptide or dodecapeptide unit. In some aspects, in the presence of an enzyme (e.g., a tumor-associated protease), an amide linkage between the amino acids is cleaved, which ultimately leads to release of free drug.

Each amino acid can be proteinogenic or non-proteinogenic and/or a D- or L-isomer provided that RL comprises a cleavable bond that, when cleaved, initiates release of the Camptothecin. In some embodiments, the Peptide Releasable Linker will comprise only proteinogenic amino acids. In some aspects, the Peptide Releasable Linker will have from 1 to no more than 12 amino acids in contiguous sequence.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, I3-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine. In some embodiments, each amino acid is selected from the proteinogenic or the non-proteinogenic amino acids.

In another embodiment, each amino acid is independently selected from the group consisting of the following L-(proteinogenic) amino acids: alanine, arginine, aspartic acid, asparagine, histidine, glycine, glutamic acid, glutamine, phenylalanine, lysine, leucine, serine, tyrosine, threonine, isoleucine, tryptophan, and valine.

In another embodiment, each amino acid is independently selected from the group consisting of the following D-isomers of these proteinogenic amino acids: alanine, arginine, aspartic acid, asparagine, histidine, glycine, glutamic acid, glutamine, phenylalanine, lysine, leucine, serine, tyrosine, threonine, isoleucine, tryptophan, and valine.

In certain embodiments, the Peptide Releasable Linker is comprised only of proteinogenic amino acids. In other embodiments, the Peptide Releasable Linker is comprised only of non-proteinogenic amino acids. In some embodiments, the Peptide Releasable Linker is comprised of a proteinogenic amino acid attached to a non-proteinogenic amino acid. In some embodiments, Peptide Releasable Linker is comprised of a proteinogenic amino acid attached to a D-isomer of a proteinogenic amino acid.

In another embodiment, each amino acid is independently selected from the group consisting of β-alanine, N-methylglycine, glycine, lysine, valine, and phenylalanine.

Exemplary Peptide Releasable Linkers include dipeptides or tripeptides such as -Val-Lys-Gly-, -Val-Cit-, -Phe-Lys-, or -Val-Ala-.

Useful Peptide Releasable Linkers can be designed and optimized in their selectivity for enzymatic cleavage by a particular enzyme, for example, a tumor-associated protease. In some embodiments, cleavage of a linkage is catalyzed by cathepsin B, C, or D, or a plasmin protease.

In some embodiments, the Peptide Releasable Linker (RL) will be represented by -(-AA-)₁₋₁₂-, or (-AA-AA-)₁₋₆ wherein AA is at each occurrence independently selected from proteinogenic or non-proteinogenic amino acids. In one aspect, AA is at each occurrence independently selected from proteinogenic amino acids. In another aspect, RL is a tripeptide having the formula: AA₁-AA₂-AA₃, wherein AA₁, AA₂ and AA₃ are each independently an amino acid and wherein AA₁ attaches to —NH- and AA₃ attaches to S. In yet another aspect, AA₃ is gly or β-ala.

In some embodiments, the Peptide Releasable Linker has the formula denoted below in the square brackets, the subscript w is an integer ranging from 1 to 12; or w is 1, 2, 3, 4, 5, 6, 7, 8, 9, 0, 11, or 12; or w is 2, 3, or 4; or w is 3; or w is 4:

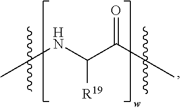

wherein R¹⁹ is, in each instance, independently selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

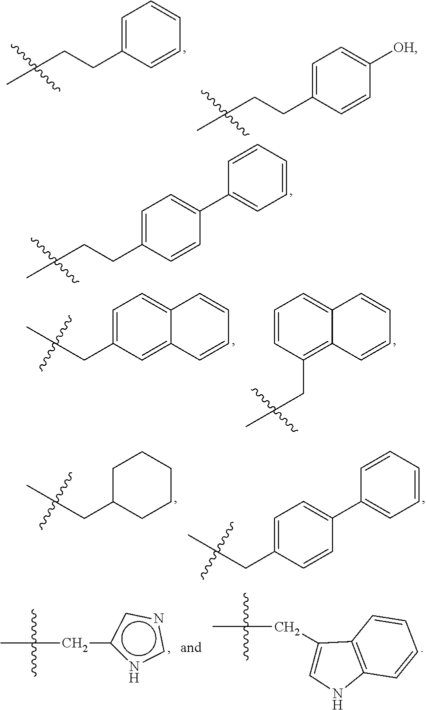

In some aspects, the subscript w is not 3.

In some aspects, each R¹⁹ is independently hydrogen, methyl, isopropyl, isobutyl, sec-butyl, —(CH₂)₃NH₂, or —(CH₂)₄NH₂. In some aspects, each R¹⁹ is independently hydrogen, isopropyl, or —(CH₂)₄NH₂.

Illustrative Peptide Releasable Linkers are represented by formulae (Pa), (Pb) and (Pc):

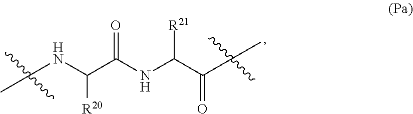

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 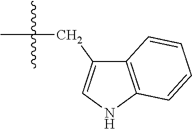 [SMILES: *Cc1c[nH]c2ccccc12] | (CH₂)₃NHCONH₂; |
| benzyl | methyl; and |
| benzyl | (CH₂)₃NHC(═NH)NH₂; |

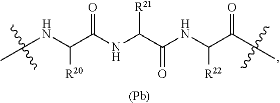

wherein R²⁰, R²¹ and R²³ are as follows:

| R²⁰ | R²¹ | R²² |
|---|---|---|
| benzyl | benzyl | —(CH₂)₄NH₂ |
| isopropyl | benzyl | —(CH₂)₄NH₂ |
| H | Benzyl | —(CH₂)₄NH₂ |
| isopropyl | —(CH₂)₄NH₂ | —H |

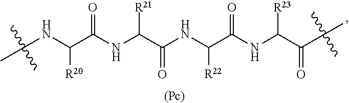

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl. |

In some embodiments, RL comprises a peptide selected from the group consisting of gly-gly, gly-gly-gly, gly-gly-gly-gly (SEQ ID NO: 1222), val-gly-gly, val-cit-gly, val-gln-gly, val-glu-gly, phe-lys-gly, leu-lys-gly, gly-val-lys-gly (SEQ ID NO: 1223), val-lys-gly-gly (SEQ ID NO: 1224), val-lys-gly, val-lys-ala, val-lys-leu, leu-leu-gly, gly-gly-phe-gly (SEQ ID NO: 1225), gly-gly-phe-gly-gly (SEQ ID NO: 1226), val-gly, and val-lys-β-ala.

In other embodiments, RL comprises a peptide selected from the group consisting of gly-gly-gly, gly-gly-gly-gly (SEQ ID NO: 1222), val-gly-gly, val-cit-gly, val-gln-gly, val-glu-gly, phe-lys-gly, leu-lys-gly, gly-val-lys-gly (SEQ ID NO: 1223), val-lys-gly-gly (SEQ ID NO: 1224), val-lys-gly, val-lys-ala, val-lys-leu, leu-leu-gly, gly-gly-phe-gly (SEQ ID NO: 1225), and val-lys-β-ala.

In still other embodiments, RL comprises a peptide selected from the group consisting of gly-gly-gly, val-gly-gly, val-cit-gly, val-gln-gly, val-glu-gly, phe-lys-gly, leu-lys-gly, val-lys-gly, val-lys-ala, val-lys-leu, leu-leu-gly and val-lys-β-ala.

In yet other embodiments, RL comprises a peptide selected from the group consisting of gly-gly-gly-gly (SEQ ID NO: 1222), gly-val-lys-gly (SEQ ID NO: 1223), val-lys-gly-gly (SEQ ID NO: 1224), and gly-gly-phe-gly (SEQ ID NO: 1225).

In other embodiments, RL is a peptide selected from the group consisting of val-gln-gly, val-glu-gly, phe-lys-gly, leu-lys-gly, val-lys-gly, val-lys-ala, val-lys-leu, leu-leu-gly and val-lys-β-ala.

In still other embodiments, RL is val-lys-gly.

In still other embodiments, RL is val-lys-β-ala.

### Glycoside Unit Releasable Linkers

In some embodiments, the Releasable Linker is a Glycoside (e.g., Glucuronide) Unit. In such embodiments, a self-immolation cascade is activated by operation of a glycosidase on a carbohydrate moiety of the Glycoside (e.g., Glucuronide) Unit. A number of sugars are useful in the embodiments described herein. Particular carbohydrate moieties include those of Galactose, Glucose, Mannose, Xylose, Arabinose, Mannose-6-phosphate, Fucose, Rhamnose, Gulose, Allose, 6-deoxy-glucose, Lactose, Maltose, Cellobiose, Gentiobiose, Maltotriose, G1cNAc, Ga1NAc, and maltohexaose.

A Glycoside (e.g., Glucuronide) Unit typically comprises a sugar moiety (Su) linked via an oxygen glycosidic bond to a self-immolative spacer. Cleavage of the oxygen glycosidic bond initiates the self-immolation reaction sequence that result in release of free drug. In some embodiments, the self-immolation sequence is activated from cleavage by β-glucuronidase of a Glycoside (e.g., Glucuronide) Unit, which is an exemplary glycoside unit. The Glycoside (e.g., Glucuronide) Unit comprises an activation unit and a self-immolative Spacer Unit. The Glycoside (e.g., Glucuronide) Unit comprises a sugar moiety (Su) linked via an oxygen glycosidic bond to a self-inunolative Spacer Unit.

In some embodiments, a Glycoside (e.g., Glucuronide) Unit comprises a sugar moiety (Su) linked via an oxygen glycoside bond (-0′-) to a Self-immolative Unit (SP) of the formula:

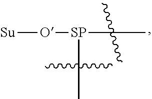

wherein the wavy lines indicate covalent attachment to the Drug Unit of any one of formula D₁ D₁, Dib, or any subformula thereof, or to a Spacer Unit that is attached to the Drug Unit (a Camptothecin Compound), and to the Stretcher Unit (Z) or its precursor (Z′), either directly or indirectly through the Connector Unit (A) or Parallel Connector Unit (B), Partitioning Agent (S*) or combinations of the Connector Unit and Parallel Connector Unit, as the case may be.

The oxygen glycosidic bond (-0′-) is typically a β-glucuronidase-cleavage site (i.e., Su is from glucuronide), such as a glycoside bond cleavable by human lysosomal β-glucuronidase.

In some embodiments, the Glycoside (e.g., Glucuronide) Unit can be represented by formula Ga, Gb, or Gc:

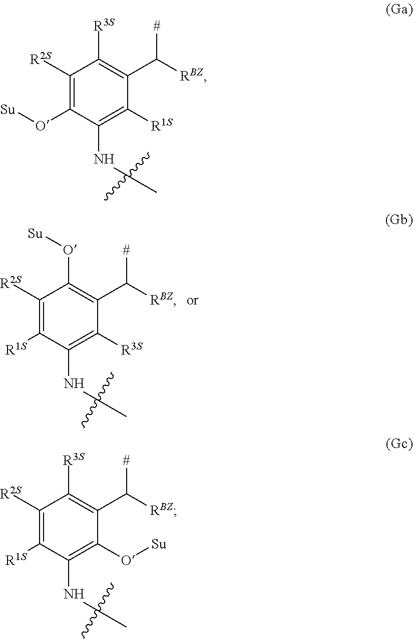

wherein Su is a Sugar moiety, —O′— represents an oxygen glycosidic bond; R^{1s}, R^{2s} and R^{3s} independently are hydrogen, a halogen, —CN, —NO₂, or other electron withdrawing group, or an electron donating group; R^{B2} is selected from the group consisting of C₁-C₆ alkyl, C₁-C₆ haloalkyl, a PEG unit, a cyclodextrin unit, a polyamide, a hydrophilic peptide, a polysaccharide, and a dendrimer, and wherein the wavy line indicates attachment to a Stretcher Unit (Z) (or its precursor (Z′)), either directly or indirectly through a Connector Unit or Parallel Connector Unit or Connector unit and Parallel Connector Unit); and # indicates attachment to the Camptothecin or to a Spacer (either directly or indirectly via an intervening functional group or other moiety).

In some embodiments, the Glycoside (e.g., Glucuronide) Unit can be represented by formula Ga*, Gb*, or Gc*:

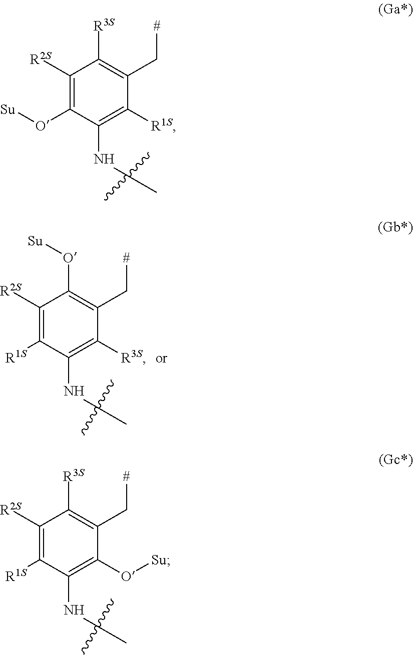

wherein Su is a Sugar moiety, —O′— represents an oxygen glycosidic bond; R^{1s}, R^{2s} and R^{3s} independently are hydrogen, a halogen, —CN, —NO₂, or other electron withdrawing group, or an electron donating group; and wherein the wavy line indicates attachment to a Stretcher Unit (Z) (or its precursor (Z′)), either directly or indirectly through a Connector Unit or Parallel Connector Unit or Connector unit and Parallel Connector Unit); and # indicates attachment to the Camptothecin or to a Spacer (either directly or indirectly via an intervening functional group or other moiety).

In some embodiments, the Glycoside (e.g., Glucuronide) Unit can be represented by formula Ga**, Gb**, or Gc**:

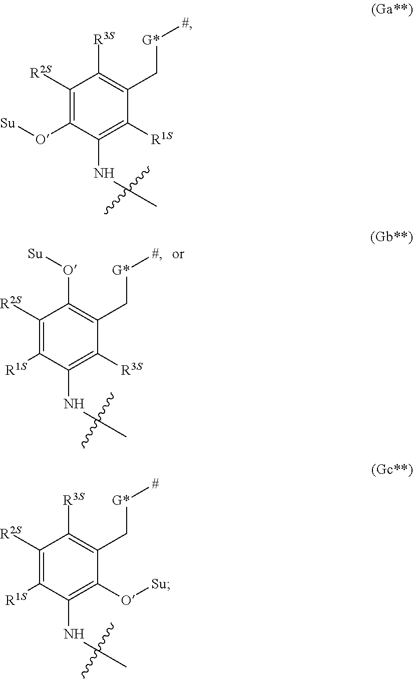

wherein Su is a Sugar moiety, —O′— represents an oxygen glycosidic bond; R^{1s}, R^{2s} and R^{3s} independently are hydrogen, a halogen, —CN, —NO₂, or other electron withdrawing group, or an electron donating group; and wherein the wavy line indicates attachment to a Stretcher Unit (Z) (or its precursor (Z′)), either directly or indirectly through a Connector Unit or Parallel Connector Unit or Connector unit and Parallel Connector Unit); # indicates attachment to the Camptothecin, optionally through a Spacer Unit; and G* is an intervening moiety comprising a functional group that is capable of attachment to the Spacer Unit or the Camptothecin. In some embodiments, the intervening moeity is —O—C(O)—.

In some embodiments, the Glycoside (e.g., Glucuronide) Unit can be represented by formula Ga***, Gb***, or Gc***:

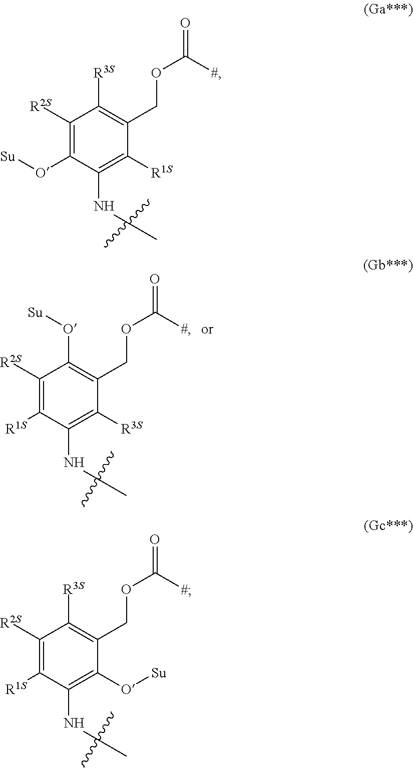

wherein Su is a Sugar moiety, —O′— represents an oxygen glycosidic bond; R¹s, R^{2s} and R^{3s} independently are hydrogen, a halogen, —CN, —NO₂, or other electron withdrawing group, or an electron donating group; and wherein the wavy line indicates attachment to a Stretcher Unit (Z) (or its precursor (Z′)), either directly or indirectly through a Connector Unit or Parallel Connector Unit or Connector unit and Parallel Connector Unit); and # indicates attachment to the Camptothecin, optionally through a Spacer Unit.

In preferred embodiments R¹s, R^{2s}, and R^{3s} are independently selected from hydrogen, halogen, —CN, or —NO₂. In other preferred embodiments, R¹s, R^{2s}, and R^{3s} are each hydrogen. In other preferred embodiments R^{2s} is an electron withdrawing group, preferably NO₂, and R^{1s} and R^{3s} are each hydrogen.

In some embodiments, the activatable self-immolative group capable of glycosidase cleavage to initiate the self-immolative reaction sequence is represented by the formula Gd:

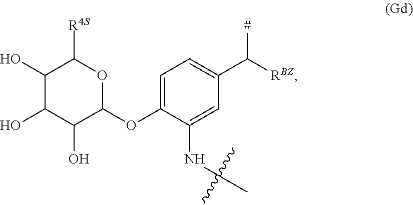

wherein R⁵ is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to the methylene carbamate unit.

In some embodiments, the activatable self-immolative group capable of glycosidase cleavage to initiate the self-immolative reaction sequence is represented by the formula Gd*:

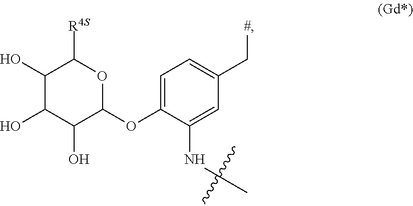

wherein R^{4s} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to a —OC(O)— unit that connects to a Spacer Unit or Camptothecin. In some embodiments, the —OC(O)— unit connects to a nitrogen atom of a Spacer Unit or Camptothecin to form a methylene carbamate moiety. In some embodiments, the —OC(O)— unit connects to an oxygen atom of a Spacer Unit or Camptothecin to form a methylene carbonate moiety.

In some embodiments, the activatable self-immolative group capable of glycosidase cleavage to initiate the self-immolative reaction sequence is represented by the formula Gd**:

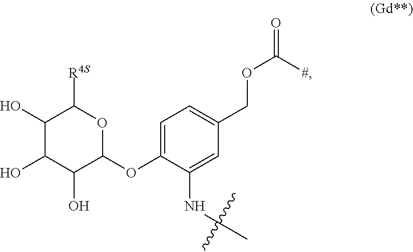

wherein R^{4s} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to Spacer Unit or the Camptothecin. In some embodiments, the —OC(O)— unit connects to a nitrogen atom of a Spacer Unit or Camptothecin to form a methylene carbomate moiety. In some embodiments, the —OC(O)— unit connects to an oxygen atom of a Spacer Unit or Camptothecin to form a methylene carbonate moiety.

In some embodiments wherein the activatable self-immolative moiety is comprised of a Glycoside (e.g., Glucuronide) Unit, the moiety is represented by the following formula Ge:

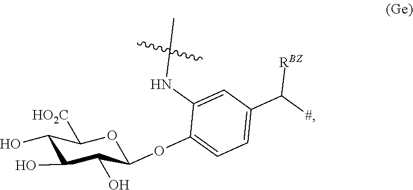

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark (#) indicates covalent attachment of the benzylic carbon of a Spacer or functional group attached to the Camptothecin. In some embodiments, the functional group is —O—C(O)—. In some embodiments, the structure of formula Ge is attached to the Drug Unit via a quaternized tertiary amine (N+), wherein the nitrogen atom is from a tertiary amine functional group on the unconjugated Drug Unit.

In some embodiments wherein the activatable self-immolative moiety is comprised of a Glycoside (e.g., Glucuronide) Unit, the moiety is represented by the following formula Ge:

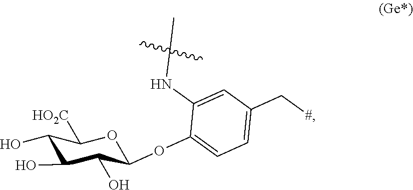

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark and # indicates attachment to the Camptothecin or to a Spacer Unit (either directly or indirectly via an intervening functional group or other moiety). In some embodiments, the intervening functional group is —O—C(O)—. In some embodiments, the structure of formula Ge is attached to the Drug Unit via a quaternized tertiary amine (N+), wherein the nitrogen atom is from a tertiary amine functional group on the unconjugated Drug Unit.

In some embodiments, the Releasable Linker has the structure:

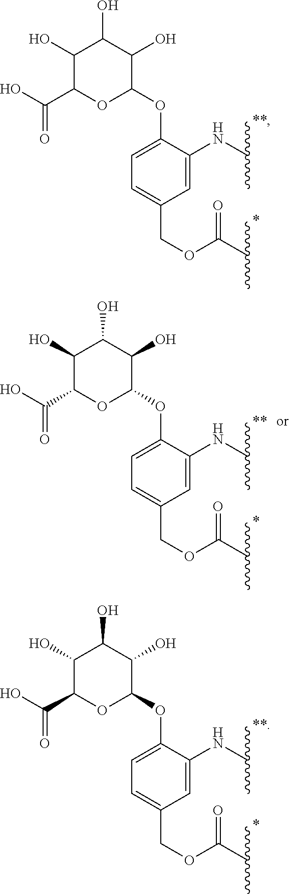 [SMILES: *Nc1cc(COC(*)=O)ccc1OC1OC(C(=O)O)C(O)C(O)C1O ; *Nc1cc(COC(*)=O)ccc1O[C@@H]1O[C@H](C(=O)O)[C@@H](O)[C@H](O)[C@H]1O ; *Nc1cc(COC(*)=O)ccc1O[C@H]1O[C@@H](C(=O)O)[C@H](O)[C@@H](O)[C@@H]1O]

In some embodiments, the Releasable Linker has the structure:

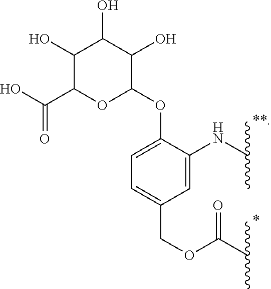 [SMILES: *Nc1cc(COC(*)=O)ccc1OC1OC(C(=O)O)C(O)C(O)C1O]

In some embodiments, the Releasable Linker has the structure:

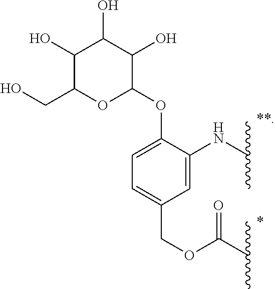 [SMILES: *Nc1cc(COC(*)=O)ccc1OC1OC(CO)C(O)C(O)C1O]

In some embodiments, the Releasable Linker has the structure:

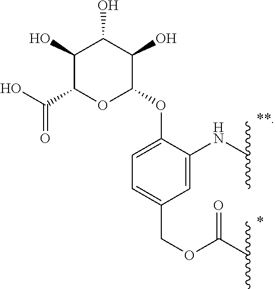 [SMILES: *Nc1cc(COC(*)=O)ccc1O[C@@H]1O[C@H](C(=O)O)[C@@H](O)[C@H](O)[C@H]1O]

In some embodiments, the Releasable Linker has the structure:

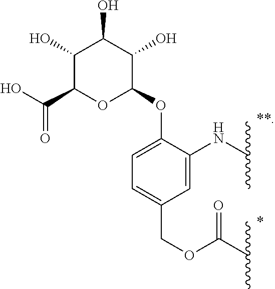 [SMILES: *Nc1cc(COC(*)=O)ccc1O[C@H]1O[C@@H](C(=O)O)[C@H](O)[C@@H](O)[C@@H]1O]

In some embodiments, the Releaseable Linker has the structure:

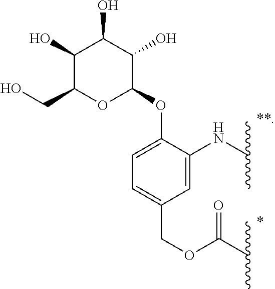 [SMILES: *Nc1cc(COC(*)=O)ccc1O[C@H]1O[C@@H](CO)[C@@H](O)[C@@H](O)[C@@H]1O]

In some embodiments, the Releaseable Linker has the structure:

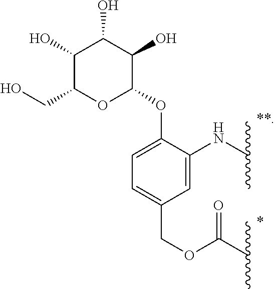 [SMILES: *Nc1cc(COC(*)=O)ccc1O[C@@H]1O[C@H](CO)[C@H](O)[C@H](O)[C@H]1O]

In some embodiments, the Releaseable Linker has the structure:

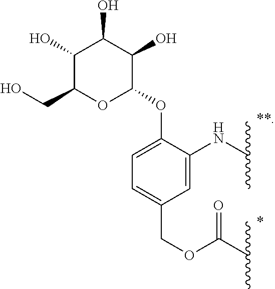 [SMILES: *Nc1cc(COC(*)=O)ccc1O[C@@H]1O[C@@H](CO)[C@H](O)[C@@H](O)[C@H]1O]

In some embodiments, the Releaseable Linker has the structure:

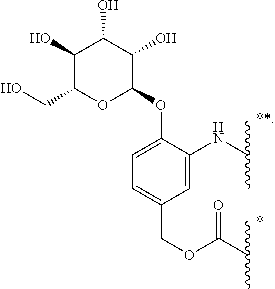 [SMILES: *Nc1cc(COC(*)=O)ccc1O[C@H]1O[C@H](CO)[C@@H](O)[C@H](O)[C@@H]1O]

Another type of Releasable Linker that provides a mechanism for separation of the Camptothecin from the Ligand Unit and other components of the Linker Unit through activation of a self-immolation cascade within the Linker Unit is comprised of a p-aminobenzyloxycarbonyl (PAB) moiety whose phenylene component is substituted with Jₘ wherein the subscript m indicating the number of substituents is an integer ranging from 0-4, and each J is independently —C₁-C₈ alkyl, —O—(C₁-C₈ alkyl), -halogen, -nitro, or -cyano.

In some embodiments, RL is a self-immolative group capable of releasing -D without the need for a separate hydrolysis step or subsequent self-immolative event. In some embodiments, -RL- is a PAB moiety that is linked to the carbonyl of -W- via the amino nitrogen atom of the PAB group, and connected directly to -D via a carbonate group. In related embodiments, -RL- is comprised of a PAB moiety that is linked to the carbonyl of -A-, -S*- or -B- via the amino nitrogen atom of the PAB group, and connected directly to -D via a carbonate group. Without being bound by any particular theory or mechanism, a possible mechanism of Drug release from RL comprised of a PAB moiety in which RL is attached directly to -D via a carbonate group is shown in Told et al. (2002) J Org. Chem. 67:1866-1872.

In some embodiments, RL units containing a PAB moiety are represented by the formula:

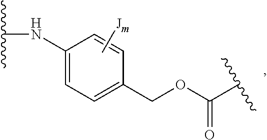

wherein subscript m is an integer ranging from 0-4, and each J is independently —C₁-C₈ alkyl, —O—(C₁-C₈ alkyl), -halogen, -nitro, or -cyano.

Other examples of self-immolative groups include, but are not limited to, aromatic compounds that are electronically similar to the PAB moiety such as 2-aminoimidazol-5-methanol derivatives (Hay et al. (1999) Bioorg. Med. Chem. Lett. 9:2237) and ortho or para-aminobenzylacetals. Other RLs undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867).

In one embodiment, RL is a branched bis(hydroxymethyl)styrene (BHMS) unit.

In some embodiments, RL has the formula:

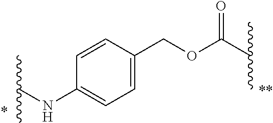 [SMILES: *Nc1ccc(COC(*)=O)cc1]

wherein the wavy line marked with ** indicates the site of attachment to D; and

the wavy line marked with * indicates the point of attachment to additional linker components of Q. In some embodiments, RL comprises the formula:

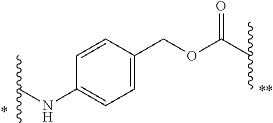 [SMILES: *Nc1ccc(COC(*)=O)cc1]

wherein the wavy line marked with ** indicates the site of attachment to D; and the wavy line marked with * indicates the point of attachment to other portions of RL, such as Peptide Releasable Linkers or Glycosidide Unit Relasable Linkers described herein.

In some embodiments, RL comprises a heterocyclic “self-immolating moiety” of Formulas I, II, or III bound to the drug and incorporates an amide group that upon hydrolysis by an intracellular protease initiates a reaction that ultimately cleaves the self-immolative moiety from the drug such that the drug is released from the conjugate in an active form. The linker moiety further comprises a peptide sequence adjacent to the self-immolative moiety that is a substrate for an intracellular enzyme, for example an intracellular protease such as a cathepsin (e.g., cathepsin B), that cleaves the peptide at the amide bond shared with the self-immolative moiety. For embodiments disclosed herein, a PAB-containing RL is directly attached to the tertiary hydroxyl of the lactone ring present in each of formula D₁ D₁, Dib, or any subformula thereof, or any of the compounds of Table I.

In some embodiments, a heterocyclic self-immolating group (RL) is selected from Formulas I, II, and III:

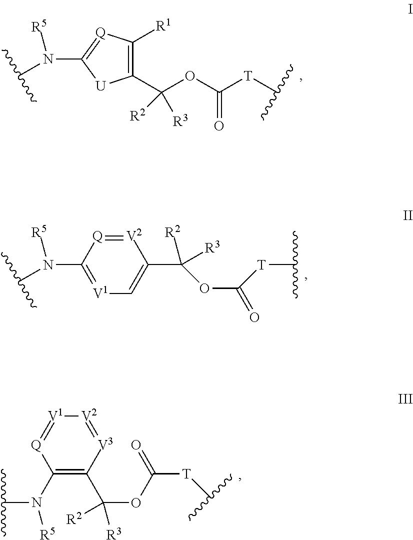

wherein the wavy lines indicate the covalent attachment sites to the cell-specific ligand and the D′ drug moiety, and wherein U is O, S or NR⁶; Q is CR⁴ or N; V¹, V², and V³ are independently CR⁴ or N provided that for formula II and III at least one of Q, V¹ and V² is N; T is the heteroatom of a hydroxyl or thiol or primary or secondary or N-heterocycle or N-amide or N-carbamate of a Drug Unit of formula D₁, D₁ₐ, or D₂, wherein T and D′ together form a Drug Unit of formula D₁ D₁ₐ, D_{1b}, or any subformula thereof, or any of the compounds of Table I; R¹, R², R³ and R⁴ are independently selected from the group consisting of H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂o substituted aryl, C₁-C₂o heterocycle, and C₁-C₂₁₃ substituted heterocycle; or when taken together, R² and R³ form a carbonyl (═O), or spiro carbocyclic ring of 3 to 7 carbon atoms; and R⁵ and R⁶ are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂o aryl, C₆-C₂o substituted aryl, C₁-C₁₀ heterocycle, and C₁-C₁₀ substituted heterocycle; wherein C₁-C₈ substituted alkyl, C₂-C₈ substituted alkenyl, C₂-C₈ substituted alkynyl, C₆-C_{2,3} substituted aryl, and C₂-C₂o substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂o aryl, C₂-C₂₀ heterocycle, polyethyleneoxy, phosphonate, and phosphate.

The conjugate can be stable extracellularly, or in the absence of an enzyme capable of cleaving the amide bond of the self-immolative moiety. However, upon entry into a cell, or exposure to a suitable enzyme, an amide bond can be cleaved initiating a spontaneous self-immolative reaction resulting in the cleavage of the bond covalently linking the self-immolative moiety to the drug, to thereby effect release of the drug in its underivatized or pharmacologically active form.

The self-immolative moiety in conjugates of the invention can either incorporate one or more heteroatoms and thereby provide improved solubility, improve the rate of cleavage, and/or decrease propensity for aggregation of the conjugate. These improvements of the heterocyclic self-immolative linker constructs of the present invention over non-heterocyclic, PAB-type linkers can in some instances result in surprising and unexpected biological properties such as increased efficacy, decreased toxicity, and/or improvements in one or more desirable pharmacokinetic and/or pharmacodynamic properties.

Not to be limited by theory or any particular mechanism, the presence of electron-withdrawing groups on the heterocyclic ring of formula I, II, or HI linkers can moderate the rate of cleavage.

In one embodiment, the self-immolative moiety is the group of formula I in which Q is N, and U is O or S. Such a group has a non-linearity structural feature which can improve the solubility of the conjugates. In this context R can be H, methyl, nitro, or CF₃. In one embodiment, Q is N and U is O thereby forming an oxazole ring and R is H. In another embodiment, Q is N and U is S thereby forming a thiazole ring optionally substituted at R with an Me or CF₃ group.

In another exemplary embodiment, the self-immolative moiety is the group of formula H in which Q is N and V¹ and V² are independently N or CH. In another embodiment, Q, V¹, and V² are each N. In another embodiment, Q and V¹ are N while V² is CH. In another embodiment, Q and V² are N while V¹ is CH. In another embodiment, Q and V¹ are both C_{H} and V² is N. In another embodiment, Q is N while V¹ and V² are both CH.

In another embodiment, the self-immolative moiety is the group of formula III in which Q, V¹, V², and V³ are each independently N or CH. In another embodiment Q is N while V¹, V², and V³ are each N. In another embodiment, Q, V¹, and V² are each C_{H} while V³ is N. In another embodiment Q, V², and V³ are each C_{H} while V¹ is N. In another embodiment, Q, V¹, and V³ are each C_{H} while V² is N. In another embodiment, Q and V² are both N while V¹ and V³ are both CH. In another embodiment Q and V² are both C_{H} while V¹ and V³ are both N. In another embodiment, Q and V³ are both N while V¹ and V² are both CH.

Without being bound by theory, Scheme 1a depicts a mechanism of free drug release from a Camptothecin Drug Unit attached through a nitrogen atom of an amine substituent from the free drug to a Releasable Linker that is a Glycoside (e.g., Glucuronide) Unit.

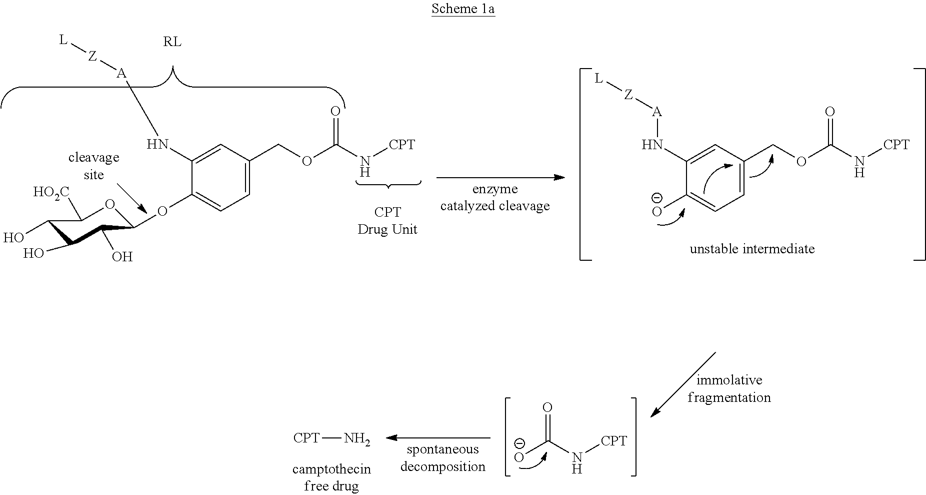

### Partitioning Agent (S′):

The Camptothecin Conjugates described herein can also include a Partitioning Agent (S*). The Partitioning Agent portions are useful, for example, to mask the hydrophobicity of particular Camptothecin Drug Units or Linking Unit components. In some embodiments, masking the hydrophobicity of the Camptothecin Drug Unit or Linking Unit improves the pharmacokinetic properties (e.g., plasma concentration over time, plasma AUC, plasma clearance rate) of the Camptothecin Conjugate. Without being bound by theory, it is believed that certain hydrophilic or amphiphilic moieties, when matched in size and/or hydrophilicity to the masked moiety's hydrophobicity and incorporated in a suitable location, can counteract negative pharmacokinetic effects caused by the hydrophobic moiety. Masking the hydrophobicity of particular Camptothecin Drug Units or Linking Unit components may allow for a corresponding Ligand Drug Conjugate to achieve higher loading (e.g., drug-antibody ratio (DAR)) compared to a similar conjugate that lacks the masking component.

Representative Partitioning Agents include polyethylene glycol (PEG) units, cyclodextrin units, polyamides, hydrophilic peptides, polysaccharides and dendrimers.

When the polyethylene glycol (PEG) units, cyclodextrin units, polyamides, hydrophilic peptides, polysaccharides or dendrimers are included in Q, the groups may be present as an ‘in line’ component or as a side chain or branched component. For those embodiments in which a branched version is present, the Linker Units can include a lysine residue (or Parallel Connector Unit, B) that provides simple functional conjugation of, for example, the PEG unit, to the remainder of the Linking Unit.

### Polyethylene Glycol Unit (PEG)

Polydisperse PEGs, monodisperse PEGs and discrete PEGs can be used as part of the Partitioning Agents in Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein can comprise one or multiple polyethylene glycol chains. A polyethylene glycol chain is composed of at least two ethylene oxide (CH₂CH₂O) subunits. In some embodiments the polyethylene glycol chains are linked together, for example, in a linear, branched or star shaped configuration. Typically, at least one of the PEG chains is derivitized at one end for covalent attachment to an appropriate site on a component of the Linker Unit (e.g. B) or can be used as an in-line (e.g., bifunctional) linking group within to covalently join two of the Linker Unit components (e.g., Z-A-S*-RL-, Z-A-S*-RL-Y-). Exemplary attachments within the Linker Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment within the Linker Unit is by means of a non-conditionally cleavable linkage. In some embodiments, attachment within the Linker Unit is not via an ester linkage, hydrazone linkage, oxime linkage, or disulfide linkage. In some embodiments, attachment within the Linker Unit is not via a hydrazone linkage.

A conditionally cleavable linkage refers to a linkage that is not substantially sensitive to cleavage while circulating in the plasma but is sensitive to cleavage in an intracellular or intratumoral environment. A non-conditionally cleavable linkage is one that is not substantially sensitive to cleavage in any biological environment. Chemical hydrolysis of a hydrazone, reduction of a disulfide, and enzymatic cleavage of a peptide bond or glycosidic linkage are examples of conditionally cleavable linkages.

In some embodiments, the PEG Unit can be directly attached to a Parallel Connector Unit B. The other terminus (or termini) of the PEG Unit can be free and untethered and may take the form of a methoxy, carboxylic acid, alcohol, or other suitable functional group. The methoxy, carboxylic acid, alcohol, or other suitable functional group acts as a cap for the terminal PEG subunit of the PEG Unit. By untethered, it is meant that the PEG Unit will not be attached at that untethered site to a Camptothecin, to an antibody, or to another linking component. The skilled artisan will understand that the PEG Unit in addition to comprising repeating ethylene glycol subunits may also contain non-PEG material (e.g., to facilitate coupling of multiple PEG chains to each other). Non-PEG material refers to the atoms in the PEG Unit that are not part of the repeating —CH₂CH₂O- subunits. In some embodiments provided herein, the PEG Unit comprises two monomeric PEG chains attached to each other via non-PEG elements. In other embodiments provided herein, the PEG Unit comprises two linear PEG chains attached to a central core or Parallel Connector Unit (i.e., the PEG Unit itself is branched).

There are a number of PEG attachment methods available to those skilled in the art, [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); PCT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal .alpha.-carbon of a peptide); Veronese et al., (1985) Appl. Biochem. Biotechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) can also be useful as a reactive group for attaching PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, PEG molecules may be attached to amino groups using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG2-succinimidyl succinate (mPEG2-SS); mPEG-succinimidyl carbonate (mPEG-SC), mPEG2-succinimidyl carbonate (mPEG2-SC); mPEG-imidate, mPEG-para-nitrophenylcarbonate (mPEG-NPC), mPEG-imidate; mPEG2-para-nitrophenylcarbonate (mPEG2-NPC); mPEG-succinimidyl propionate (mPEG-SPA); mPEG2-succinimidyl propionate (mPEG2-SPA); mPEG-N-hydroxy-succinimide (mPEG-NHS); mPEG2-N-hydroxy-succinimide (mPEG2-NHS); mPEG-cyanuric chloride; mPEG2-cyanuric chloride; mPEG2-Lysinol-NPC, and mPEG2-Lys-NHS.

Generally, at least one of the PEG chains that make up the PEG Unit is functionalized so that it is capable of covalent attachment to other Linker Unit components.

Functionalization includes, for example, via an amine, thiol, NHS ester, maleimide, alkyne, azide, carbonyl, or other functional group. In some embodiments, the PEG Unit further comprises non-PEG material (i.e., material not comprised of —CH₂CH₂O—) that provides coupling to other Linker Unit components or to facilitate coupling of two or more PEG chains.

The presence of the PEG Unit (or other Partitioning Agent) in the Linker Unit can have two potential impacts upon the pharmacokinetics of the resulting Camptothecin Conjugate. The desired impact is a decrease in clearance (and consequent increase in exposure) that arises from the reduction in non-specific interactions induced by the exposed hydrophobic elements of the Camptothecin Conjugate or to the Camptothecin itself. The second impact is undesired and is a decrease in volume and rate of distribution that sometimes arises from the increase in the molecular weight of the Camptothecin Conjugate.

Increasing the number of PEG subunits increases the hydrodynamic radius of a conjugate, typically resulting in decreased diffusivity. In turn, decreased diffusivity typically diminishes the ability of the Camptothecin Conjugate to penetrate into a tumor (Schmidt and Wittrup, Mol Cancer Ther 2009; 8:2861-2871). Because of these two competing pharmacokinetic effects, it is desirable to use a PEG that is sufficiently large to decrease the Camptothecin Conjugate clearance thus increasing plasma exposure, but not so large as to greatly diminish its diffusivity, to an extent that it interferes with the ability of the Camptothecin Conjugate to reach the intended target cell population. See the examples (e.g., examples 1, 18, and 21) of US20 1 6/03 1 06 1 2, which are incorporated by reference herein, for methodology for selecting an optimal PEG size for a particular drug-linker.

In one group of embodiments, the PEG Unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In preferred embodiments, the PEG Unit comprises a combined total of at least 4 subunits, at least 6 subunits, at least 8 subunits, at least 10 subunits, or at least 12 subunits. In some such embodiments, the PEG Unit comprises no more than a combined total of about 72 subunits, preferably no more than a combined total of about 36 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the Partitioning Agent S* is a linear PEG Unit comprising from 2 to 20, or from 2 to 12, or from 4 to 12, or 4, 8, or 12 -CH₂CH₂O— subunits. In some embodiments, the linear PEG Unit is connected at one end of the PEG Unit to the RL Unit and at the other end of the PEG Unit to the Stretcher/Connector Units (Z-A-). In some embodiments, the PEG Unit is connected to the RL Unit via a —CH₂CH₂C(O)— group that forms an amide bond with the RL Unit (e.g., —(CH₂CH₂O)n-CH₂CH₂C(O)-RL) and to the Stretcher Unit/Connector Unit (Z-A-) via an —NH— group (e.g., Z-A-NH—(CH₂CH₂O)ₙ—) that forms an amide bond with the Z-A- portion.

Illustrative embodiments for PEG Units that are connected to the RL and Stretcher/Connector Units (Z-A-) are shown below:

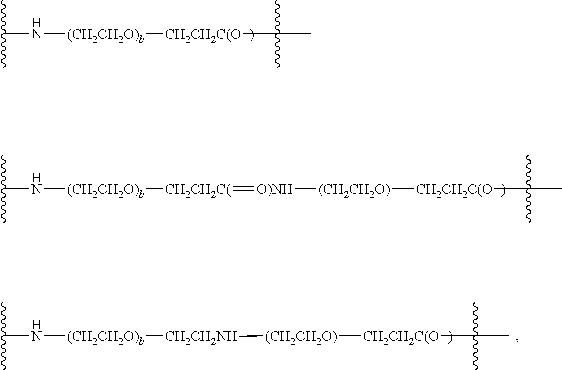

and in a particular embodiment, the PEG Unit is:

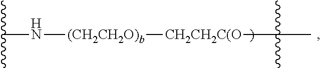

wherein the wavy line on the left indicates the site of attachment to Z-A-, the wavy line on the right indicates the site of attachment to RL, and each b is independently selected from 2 to 72, 4 to 72, 6 to 72, 8 to 72, 10 to 72, 12 to 72, 2 to 24, 4 to 24, 6 to 24, or 8 to 24, 2 to 12, 4 to 12, 6 to 12, and 8 to 12. In some embodiments, subscript b is 2, 4, 8, 12, or 24. In some embodiments, subscript b is 2. In some embodiments, subscript b is 4. In some embodiments, subscript b is 8. In some embodiments, subscript b is 12.

In some embodiments, the linear PEG Unit that is connected to the Parallel Connector Unit at one end and comprises a terminal cap at the other end. In some embodiments, the PEG Unit is connected to the Parallel Connector Unit via a carbonyl group that forms an amide bond with the Parallel Connector Unit lysine residue amino group (e.g., —(OCH₂CH₂)ₙ—C(O)—B—) and includes a PEG Unit terminal cap group selected from the group consisting of C₁₄alkyl and C₁-4alkyl-CO₂H. In some embodiments, the Partitioning Agent S* is a linear PEG Unit comprising 4, 8, or 12 -CH₂CH₂O— subunits and a terminal methyl cap.

Illustrative linear PEG Units that can be used in any of the embodiments provided herein are as follows:

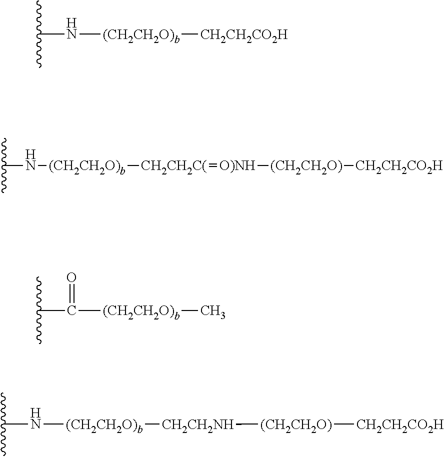

and in a particular embodiment, the PEG Unit is:

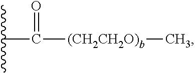

wherein the wavy line indicates site of attachment to the Parallel Connector Unit (B), and each n is independently selected from 4 to 72, 6 to 72, 8 to 72, 10 to 72, 12 to 72, 6 to 24, or 8 to 24. In some embodiments, subscript b is about 4, about 8, about 12, or about 24.

As used to herein, terms “PEG2”, “PEG4”, “PEG8”, and “PEG 12” refers to specific embodiments of PEG Unit which comprises the number of PEG subunits (i.e., the number of subscription “b”). For example, “PEG2” refers to embodiments of PEG Unit that comprises 2 PEG subunits, “PEG4” refers to embodiments of PEG Unit that comprises 4 PEG subunits, “PEG8” refers to embodiments of PEG Unit that comprises 8 PEG subunits, and “PEG 12” refers to embodiments of PEG Unit that comprises 12 PEG subunits.

As described herein, the PEG unit is selected such that it improves clearance of the resultant Camptothecin Conjugate but does not significantly impact the ability of the Conjugate to penetrate into the tumor. In embodiments, the PEG unit to be selected for use will preferably have from 2 subunits to about 24 subunits, from 4 subunits to about 24 subunits, more preferably about 4 subunits to about 12 subunits.

In preferred embodiments of the present disclosure the PEG Unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG Unit has at least 6 subunits or at least 8, 10, or 12 subunits. In some such aspects, the PEG Unit has at least 6 subunits or at least 8, 10, or 12 subunits but no more than 72 subunits, preferably no more than 36 subunits.

It will be appreciated that when referring to PEG subunits, and depending on context, the number of subunits can represent an average number, e.g., when referring to a population of Camptothecin Conjugates or Camptothecin-Linker Compounds, and/or using polydisperse PEGs.

### Parallel Connector Unit (B):

In some embodiments, the Camptothecin Conjugates and Camptothecin Linker Compounds will comprise a Parallel Connector Unit to provide a point of attachment to a Partitioning Agent (shown in the Linker Units as —B(S*)—). As a general embodiment, the PEG Unit can be attached to a Parallel Connector Unit such as lysine as shown below wherein the wavy line and asterisks indicate covalent linkage within the Linker Unit of a Camptothecin Conjugate or Camptothecin Linker Compound:

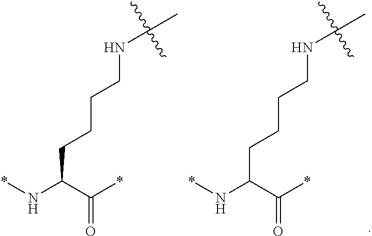 [SMILES: *NCCCC[C@H](N*)C(*)=O ; *NCCCCC(N*)C(*)=O]

In some embodiments, the Parallel Connector Unit (L^{P}) and Partitioning Agent (S*) (together, —B(S*)—) have the structure of

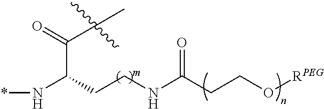

wherein m ranges from 0 to 6; n ranges from 2 to 24; R^{PEG} is a PEG Capping Unit, preferably H, —CH₃, or —CH₂CH₂CO₂H, the asterisk (*) indicates covalent attachment to a Connector Unit A corresponding in formula Za, Za′, Zb′ or Zc′ and the wavy line indicates covalent attachment to the Releasable Linker (RL). In some embodiments, the structure is attached to a Connector Unit A in formula Za or Za′. In some embodiments, n is 2, 4, 8, or 12. In instances such as those shown here, the shown PEG group is meant to be exemplary of a variety of Partitioning Agents including PEG groups of different lengths and other Partitioning Agents that can be directly attached or modified for attachment to the Parallel Connector Unit.

Spacer Unit (Y):

In some embodiments, the Camptothecin Conjugates provided herein will have a Spacer (Y) between the Releasable Linker (RL) and the Drug Unit. The Spacer Unit can be a functional group to facilitate attachment of RL to the Drug Unit, or it can provide additional structural components to further facilitate release of the Drug Unit from the remainder of the Conjugate (e.g., a methylene carbamate unit or a self-immolative para-aminobenzyl (PAB) component).

In those embodiments to further facilitate release of the Drug Unit as free drug, the Spacer Unit-Drug Unit group (-Y-T*-D or -Y-D) is represented by one of the following formulae:

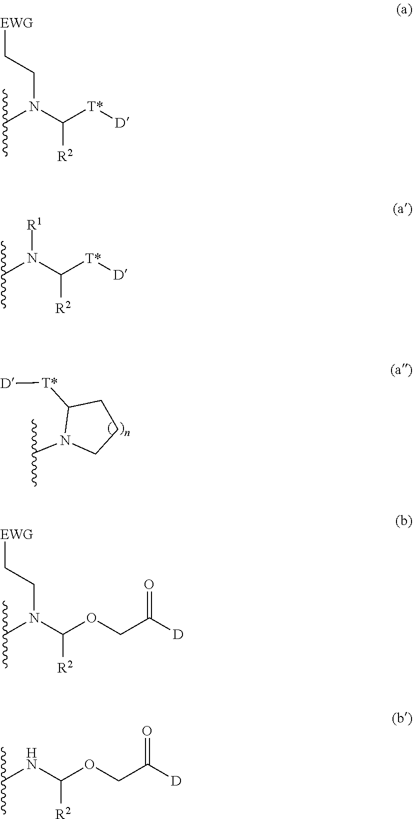

wherein R¹ and R² are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂o aryl, C₆-C₂₀ substituted aryl, C₁-C₁₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂₀ substituted heterocycle are independently optionally substituted with one or more substituents selected from the group consisting of F, Cl, Br, I, OH, —N(R⁵)₂, —N(R⁵)₃+, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂⁻C₂o heterocycle, polyethyleneoxy, phosphonate, and phosphate; subscript n is 1 or 2; wherein EWG represents an electron-withdrawing group; T* is the heteroatom of a hydroxyl or thiol or primary or secondary amine or N-heterocycle or N-amide or N-cast amate of a Drug Unit of formula D₀, D₁ D₁ₐ, D_{1b}, or any subformula thereof; D′ is the remainder of a Drug Unit, wherein T* and D′ together form a Drug Unit of formula D₀, D₁ D₁ₐ, D_{1b}, or any subformula thereof (i.e. T*+D′=D); D is a Drug Unit of formula D₀, D₁ D₁ₐ, D_{1b}, or any subformula thereof; and the wavy line adjacent to the nitrogen atom is the point of covalent attachment to RL. In some embodiments, EWG is selected from the group consisting of —CN, —NO₂, —CX₃, —X, —C(═O)OR′, —C(═O)N(R′)₂, —C(═O)R′, —C(═O)X, —S(═O)₂R, —S(═O)₂₀R, —S(═O)₂NHR′, —S(═O)₂N(R′)₂, —P(═O)(0R)₂, —P(═OXCH₃)NHR′, —NO, —N(R′)₃⁺, wherein X is —F, —Br, —Cl, or —I, and R′ is independently selected from the group consisting of hydrogen and C₁-C₆ alkyl.

In some embodiments, the Spacer Unit is represented by one of the following formulae: SO₂Me

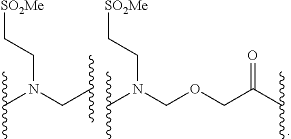

In still other embodiments, the Spacer Unit-Drug Unit group (-Y-T*-D or -Y-D) comprises a methylene carbamate unit and is represented by one of the following the formulae:

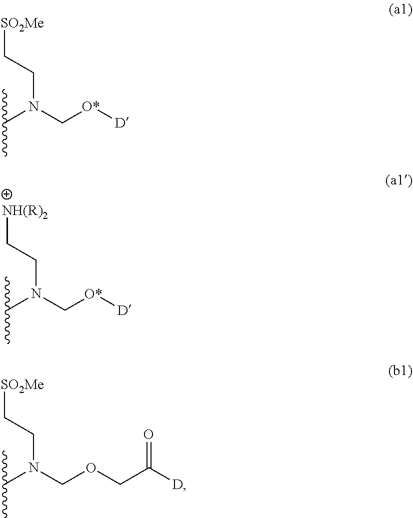

wherein formula (al) and formula (al′) in which each R is independently —H or C₁-C₄ alkyl represents Spacer Units in which O* is the oxygen atom from the hydroxyl substituent to the lactone ring of a Drug Unit of formula Do, D₁ D₁, Dib, or any subformula thereof, or of any one of compounds of Table I, and the wavy lines of formula (al), formula (al′) and formula (b 1) retain their previous meanings from formulae (a), (a′) and (b), respectively. In formula (al′) the —CH₂CH₂N+(R)₂ moiety represents exemplary Basic Units in protonated form.

In some embodiments, the Spacer Unit-Drug Unit group -Y-T*-D′ is represented by one of the following formulae:

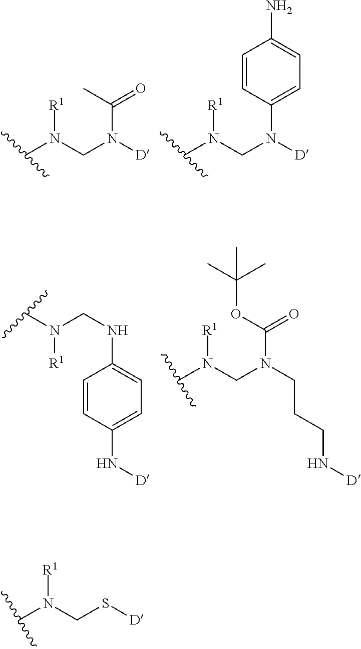

wherein R¹ is as defined for formula (a′), the wavy line adjacent to the nitrogen atom is the point of covalent attachment to RL, T* is as defined above, and D′ represents the remainder of the Drug Unit, wherein T* and D′ together form a Drug Unit of formula Do, D₁ D₁, Dib, or any subformula thereof.

In some embodiments, the Spacer Unit-Drug Unit group (-Y-T*-D) is represented by one of the following formulae:

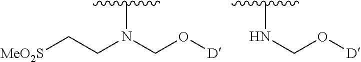

wherein the wavy line adjacent is the point of covalent attachment to RL, T* is as defined above, and D′ represents the remainder of the Drug Unit, wherein T* and D′ together form a Drug Unit of formula Do, D₁ D₁, Dib, or any subformula thereof.

In some embodiments, the Spacer Unit-Drug Unit group (-Y-T*-D) is represented by one of the following formulae:

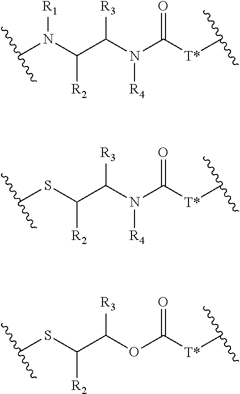

wherein R¹ and R⁴ are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂o aryl, C₆-C₂₀ substituted aryl, C₁-C₁₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂⁻C₂₀ substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate; R² is selected from the group consisting of H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₁₀ heterocycle, and C₁-C₁₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂o substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂o heterocycle, polyethyleneoxy, phosphonate, and phosphate, or is combined with R³ and the intervening atoms to form a 5- or 6-membered carbocyclo or heterocyclo; R³ is selected from the group consisting of H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₁₀ heterocycle, and C₁-C₁₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂o substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂o heterocycle, polyethyleneoxy, phosphonate, and phosphate, or is combined with R² and the intervening atoms to form a 5- or 6-membered carbocyclo or heterocyclo; T* is the heteroatom of a hydroxyl or thiol or primary or secondary amine or N-heterocycle or N-amide or N-carbamate of a Drug Unit of formula D₀, D₁ D₁ₐ, D_{1b}, or any subformula thereof; D′ is the remainder of a Drug Unit, wherein T* and D′ together form a Drug Unit of formula Do, D₁ D₁ₐ, D_{1b}, or any subformula thereof (i.e. T*+D′=D); D is a Drug Unit of formula D₁, D₁ D₁ₐ, D_{1b}, or any subformula thereof; and the wavy line adjacent to the nitrogen atom is the point of covalent attachment to RL.

In some embodiments, the Spacer Unit-Drug Unit (-Y-T*-D′) is represented by one of the following formulae:

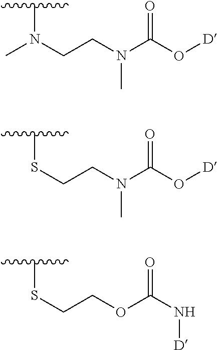

wherein the wavy line adjacent to the nitrogen atom is the point of covalent attachment to RL, T* is as defined above, and D′ represents the remainder of the Drug Unit, wherein T* and D′ together form a Drug Unit of formula Do, D₁ D₁, Dib, or any subformula thereof.

In some embodiments, the Spacer Unit is represented by the formula:

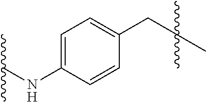 [SMILES: *Cc1ccc(N*)cc1]

wherein the wavy line adjacent to the nitrogen atom is the point of covalent attachment to RL, as defined above, and the wavy line next to the benzylic carbon atom connects to a Drug Unit. In some embodiments, the Drug Unit is attached to the benzylic carbon atom via a quaternized tertiary amine (N+) of D.

In still other embodiments, the Spacer Unit is represented by the formula:

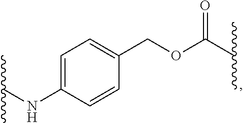 [SMILES: *Nc1ccc(COC(*)=O)cc1]

wherein the wavy line adjacent to the nitrogen atom is the point of covalent attachment to RL, as defined above, and the wavy line next to the —OC(O)— group connects to a Drug Unit. In some embodiments, the Drug Unit is attached via T*, wherein T* is the heteroatom of a hydroxyl or thiol or primary amine, secondary amine, or N-heterocycle or N-amide or N-carbamate of a Drug Unit of formula D₀, D₁ D₁ₐ, D_{1b}, or any subformula thereof.
Subscript “p”

In one group of embodiments of the invention, subscript p represents the number of Drug Linker moieties on a Ligand Unit of an individual Camptothecin Conjugate and is an integer preferably ranging from 1 to 16, 1 to 12, 1 to 10, or 1 to 8. Individual Camptothecin Conjugates can be also be referred to as a Camptothecin Conjugate compound. In any of the embodiments herein, there can be 1, 2, 3, 4, 5, 6, 7, 8, 9, 10, 11, 12, 13, 14, 15, or 16 Drug Linker moieties conjugated to a Ligand Unit of an individual Camptothecin Conjugate. In another group of embodiments of the invention, a Camptothecin Conjugate describes a population of individual Camptothecin Conjugate compounds substantially identical except for the number of Camptothecin Drug-Linker moieties bound to each Ligand Unit (i.e., a Camptothecin Conjugate composition) so that subscript p represents the average number of Camptothecin drug linker moieties bound to the Ligand Units of the Camptothecin Conjugate composition. In that group of embodiments, subscript p is a number ranging from 1 to about 16, 1 to about 12, 1 to about 10, or 1 to about 8, from 2 to about 16, 2 to about 12, 2 to about 10, or 2 to about 8. In some embodiments, p is about 2. In some embodiments, p is about 4. In some embodiments, p is about 8. In some embodiments, p is about 16. In some embodiments, p is 2. In some embodiments, p is 4. In some embodiments, p is 8. In some embodiments, p is 16. In some embodiments, the value of subscript p refers to the average drug loading as well as the drug loading of the predominate ADC in the composition.

In some embodiments, conjugation will be via the interchain disulfides and there will from 1 to about 8 Camptothecin Linker Compound molecules conjugated to a targeting agent that becomes a Ligand Unit. In some embodiments, conjugation will be via an introduced cysteine residue as well as interchain disulfides and there will be from 1 to 10 or 1 to 12 or 1 to 14 or 1 to 16 Camptothecin Linker Compound moieties conjugated to a Ligand Unit. In some embodiments, conjugation will be via an introduced cysteine residue and there will be 2 or 4 Camptothecin Linker Compound molecules conjugated to a Ligand Unit.

### Partially Released Free Drug

In some embodiments, are compounds where the RL unit in the conjugate has been cleaved, leaving the drug moiety with one amino acid residue bound thereto. In some embodiments, the partially released Free Drug (Drug-Amino Acid Conjugate) is a compound of Formula (IV):

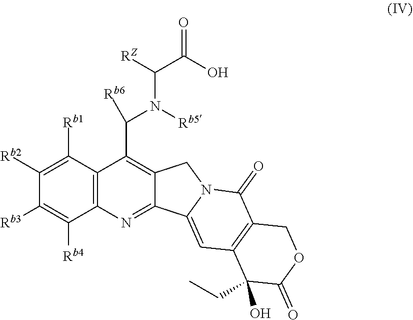

or a stereoisomer or mixture of stereoisomers thereof, or a pharmaceutically acceptable salt thereof, wherein R^{z} is an amino acid sidechain as described herein. In some embodiments, R^{x}′ is H, methyl, isopropyl, benzyl, or —(CH₂)₄—NH₂. In some embodiments, R^{z} is H or methyl. In some embodiments, R^{z} is H. In some embodiments, R^{z} is methyl.

In some embodiments, the compound of Formula (IV) is a biologically active compound. In some embodiments, such compounds are useful in a method of inhibiting topoisomerase, killing tumor cells, inhibiting growth of tumor cells, cancer cells, or of a tumor, inhibiting replication of tumor cells or cancer cells, lessening of overall tumor burden or decreasing the number of cancerous cells, or ameliorating one or more symptoms associated with a cancer or autoimmune disease. Such methods comprise, for example, contacting a cancer cell with a compound of Formula (N).

### Camptothecin Conjugate Mixtures and Compositions

The present invention provides Camptothecin Conjugate mixtures and pharmaceutical compositions comprising any of the Camptothecin Conjugates described herein. The mixtures and pharmaceutical compositions comprise a plurality of conjugates. In some embodiments, each of the conjugates in the mixture or composition is identical or substantially identical, however, the distribution of drug-linkers on the ligands in the mixture or compositions may vary as well as the drug loading. For example, the conjugation technology used to conjugate drug-linkers to antibodies as the targeting agent in some embodiments results in a composition or mixture that is heterogeneous with respect to the distribution of Camptothecin Linker Compounds on the antibody (Ligand Unit) within the mixture and/or composition. In some of those embodiments, the loading of Camptothecin Linker Compounds on each of the antibody molecules in a mixture or composition of such molecules is an integer that ranges from 1 to 16.

In those embodiments, when referring to the composition as a whole, the loading of drug-linkers is a number ranging from 1 to about 16. Within the composition or mixture, there sometimes is a small percentage of unconjugated antibodies. The average number of drug-linkers per Ligand Unit in the mixture or composition (i.e., average drug-load) is an important attribute as it determines the maximum amount of drug that can be delivered to the target cell. Typically, the average drug load is 1, 2 or about 2, 3 or about 3, 4 or about 4, 5 or about 5, 6 or about 6, 7 or about 7, 8 or about 8, 9 or about 9, 10 or about 10, 11 or about 11, 12 or about 12, 13 or about 13, 14 or about 14, 15 or about 15, 16 or about 16.

In some embodiments, the mixtures and pharmaceutical compositions comprise a plurality (i.e., population) of conjugates, however, the conjugates are identical or substantially identical and are substantially homogenous with respect to the distribution of drug-linkers on the ligand molecules within the mixture and/or composition and with respect to loading of drug-linkers on the ligand molecules within the mixture and/or composition. In some such embodiments, the loading of drug-linkers on an antibody Ligand Unit is 2 or 4. Within the composition or mixture, there may also be a small percentage of unconjugated antibodies. The average drug load in such embodiments is about 2 or about 4. Typically, such compositions and mixtures result from the use of site-specific conjugation techniques and conjugation is due to an introduced cysteine residue.

The average number of Camptothecins or Camptothecin-Linker Compounds per Ligand Unit in a preparation from a conjugation reaction may be characterized by conventional means such as mass spectrometry, ELISA assay, HPLC (e.g., HIC). In those instances, the quantitative distribution of Camptothecin Conjugates in terms of subscript p may also be determined. In other instances, separation, purification, and characterization of homogeneous Camptothecin Conjugates may be achieved by conventional means such as reverse phase HPLC or electrophoresis.

In some embodiments, the compositions are pharmaceutical compositions comprising the Camptothecin Conjugates described herein and a pharmaceutically acceptable carrier. In some of those embodiments, the pharmaceutical composition is in liquid form. In some embodiments, the pharmaceutical composition is a solid. In other of those embodiments, the pharmaceutical composition is a lyophilized powder.

The compositions, including pharmaceutical compositions, can be provided in purified form. As used herein, “purified” means that when isolated, the isolate contains at least 95%, and in other embodiments at least 98% of Conjugate by weight of the isolate.

### Methods of Use

Treatment of Cancer

The Camptothecin Conjugates are useful for inhibiting the multiplication of a tumor cell or cancer cell, causing apoptosis in a tumor or cancer cell, or for treating a cancer in a patient. The Camptothecin Conjugates are used accordingly in a variety of settings for the treatment of cancers. The Camptothecin Conjugates are intended to deliver a drug to a tumor cell or cancer cell. Without being bound by theory, in one embodiment, the Ligand Unit of a Camptothecin Conjugate binds to or associates with a cancer-cell or a tumor-cell-associated antigen, and the Camptothecin Conjugate is taken up (internalized) inside the tumor cell or cancer cell through receptor-mediated endocytosis or other internalization mechanism. In some embodiments, the antigen is attached to a tumor cell or cancer cell or is an extracellular matrix protein associated with the tumor cell or cancer cell. Once inside the cell, via activation of the Activation Unit, the drug is released within the cell. In an alternative embodiment, the free drug is released from the Camptothecin Conjugate outside the tumor cell or cancer cell, and the free drug subsequently penetrates the cell.

In one embodiment, the Ligand Unit binds to the tumor cell or cancer cell.

In another embodiment, the Ligand Unit binds to a tumor cell or cancer cell antigen which is on the surface of the tumor cell or cancer cell.

In another embodiment, the Ligand Unit binds to a tumor cell or cancer cell antigen that is an extracellular matrix protein associated with the tumor cell or cancer cell.

The specificity of the Ligand Unit for a particular tumor cell or cancer cell is an important consideration for determining the tumors or cancers that are most effectively treated. For example, Camptothecin Conjugates that target a cancer cell antigen present on hematopoietic cancers are useful treating hematologic malignancies (e.g., anti-CD30, anti-CD70, anti-CD19, anti-CD33 binding Ligand Unit (e.g., antibody) are useful for treating hematologic malignancies). Camptothecin Conjugates that target a cancer cell antigen present on solid tumors in some embodiments are useful treating such solid tumors.

Cancers that are intended to be treated with a Camptothecin Conjugate include, but are not limited to, hematopoietic cancers such as, for example, lymphomas (Hodgkin Lymphoma and Non-Hodgkin Lymphomas) and leukemias and solid tumors. Examples of hematopoietic cancers include, follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, neuroblastoma, and retinoblastoma.

In preferred embodiments, the treated cancer is any one of the above-listed lymphomas and leukemias.

### Multi-Modality Therapy for Cancer

Cancers, including, but not limited to, a tumor, metastasis, or other disease or disorder characterized by uncontrolled cell growth are intended to be treated or inhibited by administration of an effective amount of a Camptothecin Conjugate.

In one group of embodiments, methods for treating cancer are provided, including administering to a patient in need thereof an effective amount of a Camptothecin Conjugate and a chemotherapeutic agent. In one embodiment the chemotherapeutic agent is one in which treatment of the cancer has not been found to be refractory to that agent. In another embodiment, the chemotherapeutic agent one in which the treatment of cancer has been found to be refractory to that agent.

In another group of embodiments, the Camptothecin Conjugate is administered to a patient that has also undergone surgery as treatment for the cancer. In such embodiments a chemotherapeutic agent is typically administered over a series of sessions, or one or a combination of the chemotherapeutic agents, such a standard of care chemotherapeutic agent(s), is administered.

In either group of embodiments, the patient also receives an additional treatment, such as radiation therapy. In a specific embodiment, the Camptothecin Conjugate is administered concurrently with the chemotherapeutic agent or with radiation therapy. In another specific embodiment, the chemotherapeutic agent or radiation therapy is administered prior or subsequent to administration of a Camptothecin Conjugate.

Additionally, methods of treatment of cancer with a Camptothecin Conjugate are provided as an alternative to chemotherapy or radiation therapy where the chemotherapy or the radiation therapy has proven or can prove too toxic, e.g., results in unacceptable or unbearable side effects, for the subject being treated. The patient being treated is optionally treated with another cancer treatment such as surgery, radiation therapy or chemotherapy, depending on which treatment is found to be acceptable or bearable.

### Treatment of Autoimmune Diseases

The Camptothecin Conjugates are intended to be useful for killing or inhibiting the unwanted replication of cells that produce an autoimmune disease or for treating an autoimmune disease.

The Camptothecin Conjugates are used accordingly in a variety of settings for the treatment of an autoimmune disease in a patient. The Camptothecin Conjugates are typically used to deliver a camptothecin drug to a target cell. Without being bound by theory, in one embodiment, the Camptothecin Conjugate associates with an antigen on the surface of a pro-inflammatory or inappropriately stimulated immune cell, and the Camptothecin Conjugate is then taken up inside the targeted cell through receptor-mediated endocytosis. Once inside the cell, the Linker Unit is cleaved, resulting in release of the Camptothecin Drug Unit as free drug. The Camptothecin free drug is then able to migrate within the cytosol and induce a cytotoxic or cytostatic activity. In an alternative embodiment, the Camptothecin Drug Unit is cleaved from the Camptothecin Conjugate outside the target cell, and the Camptothecin free drug resulting from that release subsequently penetrates the cell.

In one embodiment, the Ligand Unit binds to an autoimmune antigen. In one such embodiment, the antigen is on the surface of a cell involved in an autoimmune condition.

In one embodiment, the Ligand Unit binds to activated lymphocytes that are associated with the autoimmune disease state.

In a further embodiment, the Camptothecin Conjugate kills or inhibits the multiplication of cells that produce an autoimmune antibody associated with a particular autoimmune disease.

Particular types of autoimmune diseases intended to be treated with the Camptothecin Conjugates include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

### Multi-Drug Therapy of Autoimmune Diseases

Methods for treating an autoimmune disease are also disclosed including administering to a patient in need thereof an effective amount of a Camptothecin Conjugate and another therapeutic agent known for the treatment of an autoimmune disease.

### Compositions and Methods of Administration

The present invention provides pharmaceutical compositions comprising the Camptothecin Conjugates described herein and at least one pharmaceutically acceptable carrier. The pharmaceutical composition is in any form that allows the compound to be administered to a patient for treatment of a disorder associated with expression of the antigen to which the Ligand unit binds. For example, the conjugates are in the form of a liquid or solid. The preferred route of administration is parenteral. Parenteral administration includes subcutaneous injections, intravenous, intramuscular, intrasternal injection or infusion techniques. In one embodiment, the pharmaceutical compositions is administered parenterally. In one embodiment, the conjugates are administered intravenously. Administration is by any convenient route, for example by infusion or bolus injection.

Pharmaceutical compositions are formulated to allow a Camptothecin Conjugate to be bioavailable upon administration of the composition to a patient. Compositions sometimes take the form of one or more dosage units.

Materials used in preparing the pharmaceutical compositions are preferably non toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

The composition in some embodiments is in the form of a liquid. The liquid in some of those embodiments is useful for delivery by injection. In some embodiments a composition for administration by injection, in addition to the Camptothecin Conjugate, contains one or more excipients selected from the group consisting of a surfactant, preservative, wetting agent, dispersing agent, suspending agent, buffer, stabilizer and isotonic agent.

The liquid compositions, whether they are solutions, suspensions or other like form, in some embodiments include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is sometimes enclosed in ampoule, a disposable syringe or a multiple-dose vial made of glass, plastic or other material. Physiological saline is an exemplary adjuvant. An injectable composition is preferably sterile.

The amount of the conjugate that is effective in the treatment of a particular disorder or condition will depend on the nature of the disorder or condition, which in some embodiments is determined by standard clinical techniques. In addition, in vitro or in vivo assays are optionally employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a Camptothecin Conjugate such that a suitable dosage amount will be obtained. Typically, that amount is at least about 0.01% of a compound by weight of the composition.

For intravenous administration, the pharmaceutical composition typically comprises from about 0.01 to about 100 mg of a Camptothecin Conjugate per kg of the animal's body weight. In one embodiment, the composition can include from about 1 to about 100 mg of a Camptothecin Conjugate per kg of the animal's body weight. In another aspect, the amount administered will be in the range from about 0.1 to about 25 mg/kg of body weight of a compound. Depending on the drug used, the dosage can be even lower, for example, 1.0 μg/kg to 5.0 mg/kg, 4.0 mg/kg, 3.0 mg/kg, 2.0 mg/kg or 1.0 mg/kg, or 1.0 μg/kg to 500.0 μg/kg of the subject's body weight.

Generally, the dosage of a conjugate administered to a patient is typically about 0.01 mg/kg to about 100 mg/kg of the subject's body weight or from 1.0 μg/kg to 5.0 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is between about 0.1 to 4 mg/kg, even more preferably 0.1 to 3.2 mg/kg, or even more preferably 0.1 to 2.7 mg/kg of the subject's body weight over a treatment cycle.

The term “carrier” refers to a diluent, adjuvant or excipient, with which a compound is administered. Such pharmaceutical carriers in some embodiments is a liquid, such as water and oils, including those of petroleum, animal, vegetable or synthetic origin, such as peanut oil, soybean oil, mineral oil, sesame oil. Other carriers include saline, gum acacia, gelatin, starch paste, talc, keratin, colloidal silica, urea. In addition, auxiliary, stabilizing, thickening, lubricating and coloring agents are sometimes used. In one embodiment, when administered to a patient, the Camptothecin Conjugate or compositions thereof and pharmaceutically acceptable carriers are sterile.

Water is an exemplary carrier when the compounds are administered intravenously. Saline solutions and aqueous dextrose and glycerol solutions are often employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol. The present compositions, if desired, also contain minor amounts of wetting or emulsifying agents, or pH buffering agents.

In an embodiment, the conjugates are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions include a solubilizing agent. Compositions for intravenous administration optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where a conjugate is to be administered by infusion, it is typically dispensed, for example, with an infusion bottle containing sterile pharmaceutical grade water or saline. Where the conjugate is administered by injection, an ampoule of sterile water for injection or saline is sometimes provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

### Methods of Preparing Camptothecin Conjugates

The Camptothecin Conjugates described herein are prepared in either a serial construction of antibodies, linkers, and drug units, or in a convergent fashion by assembling portions followed by a completed assembly step. The Curtius Rearrangement or a Chloramine synthesis can be used to provide a methylene carbamate linker (Spacer) which is useful in a number of embodiments of the Conjugates described herein.

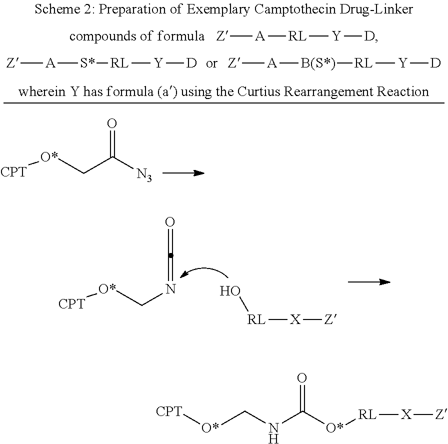

Scheme 2 illustrates a synthetic strategy involving a Curtius rearrangement of an acyl azide derivative of the free drug, wherein CPT is a Camptothecin Drug Unit corresponding in structure to a Camptothecin compound having a hydroxyl functional group, such as one of formula D₁ D₁, Dib, or any subformula thereof, or any of the compounds of Table I, whose oxygen atom, which is represented by O*, is incorporated into the methylene carbamate unit formed as a consequence of the rearrangement, Z′ is a Stretcher Unit precursor, RL is a Releasable Linker and X is -A-, -A-S*- or -A-B(S*)- wherein A is a Connector Unit, S* is a Partitioning agent and B is a Parallel Connector Unit. That strategy may be applied to Camptothecin drugs containing multiple alcohols, or other heteroatoms, as a means for acquiring regioselectivity, as there a many complementary methods of alkylation to form an acyl azide such as: halo ester alkylation, halo acid alkylation or metal carbene insertion with ethyl or methyl diazoacetate, see Doyle, M. et al. Modern Catalytic Methods for Organic Synthesis with Diazo Compounds; Wiley: New York, 1998. The acyl azide is then heated with at least a stoichiometric amount of alcohol-containing Linker Unit intermediate of formula Z′-X-RL-OH.

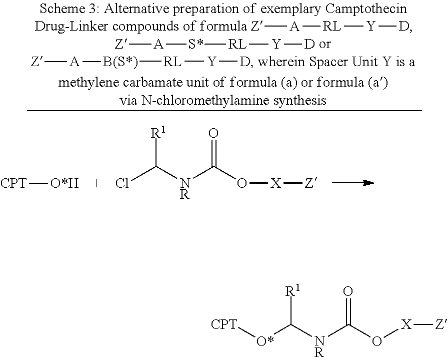

wherein R¹ is hydrogen or C₁-C₄ alkyl, R is —H or —CH₂CH₂SO₂Me and the other the variable groups have their meanings from Scheme 2.

The N-chloromethylamine synthesis is an alternative to the Curtius rearrangement in that it allows for the introduction of an unmodified alcohol or other heteroatom containing Camptothecin compound, whose use may not be compatible with the conditions required to form the acyl azide of Scheme 2, and proceeds by condensation with a reactive N-chloromethylamine. That methodology is also more appropriate for introducing certain types of methylene carbamate units as shown for example by Scheme 4.

Scheme 4 demonstrates synthesis of exemplary Camptothecin-Linker Compounds of formula Z′-A-RL-Y-D, Z′-A-S*-RL-Y-D or Z′-A-B(S*)-RL-Y-D wherein the Spacer Unit (Y) is a methylene carbamate unit of formula (a″). Reaction of the p-nitro-phenyl carbonate with the cyclic aminol provides a carbamate, which is then converted to the chlorcycloalkylamine for alkylation with a nucleophile from the thiol, hydroxyl, amine or amide functional group of free camptothecin drug. Alternatively, the carbamate can be treated with acid in the presence of the drug moiety to assemble the drug-linker intermediate shown. The alkylation product is deprotected followed by condensation of the resulting free amine with 3-maleimidopropionic acid N-hydroxysuccimide ester, which introduces a Stretcher Unit precursor covalently attached to a Connector Unit thus providing Camptothecin-Linker Compounds. The resulting Camptothecin-Linker Compounds are then condensed with a thiol-containing targeting agent to provide Camptothecin Conjugates having a Spacer Unit comprising a self-immolative moiety and the methylene carbamate unit of formula a″.

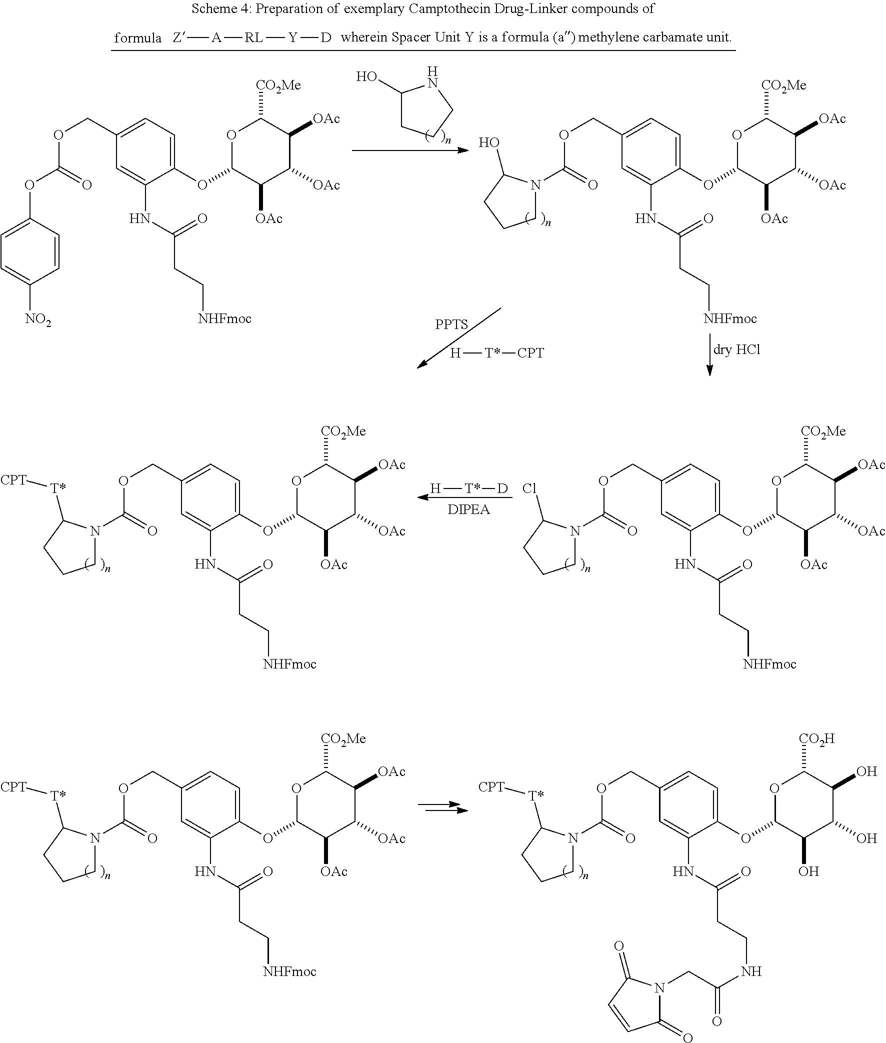

For Camptothecin-Linker Compounds and Camptothecin Conjugates having a methylene carbamate unit wherein T* is the nitrogen atom from a primary or secondary amine substituent of a Camptothecin compound direct alkylation with a chlormethylamine following the generalized procedures provided by Scheme 3 or Scheme 4 may not be suitable due to excessive or undesired over-alkylation of the nitrogen heteroatom from the amine functional group of free drug. In those instances, the method embodied by Scheme 5 may be used.

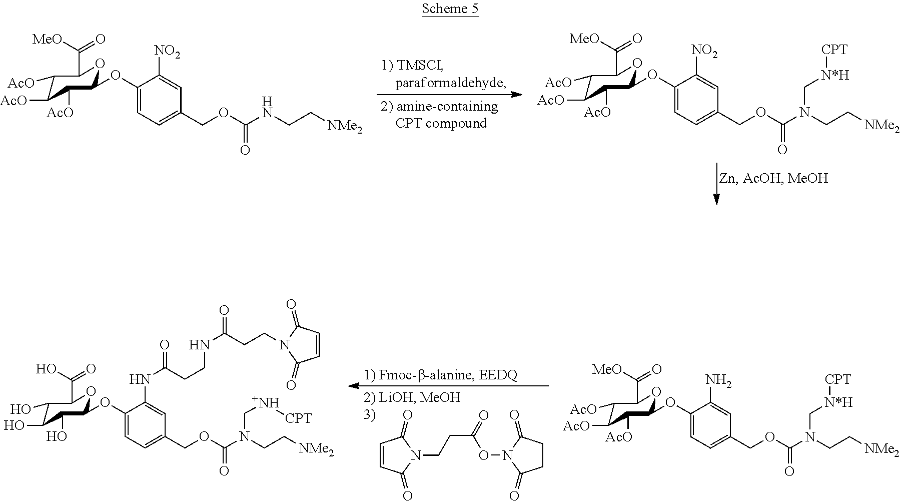

In Scheme 5 an intermediate carbamate is prepared already having a Basic Unit (i.e., the dimethylaminoethyl moiety) as the R substituent for a formula (al′) methylene carbamate unit. The nitrogen of that carbamate is condensed with formaldehyde and the resulting intermediate quenched with the amine functional group of an aliphatic amine-containing camptothecin drug. N* represents the nitrogen atom from that functional group. That condensation forms the methylene carbamate of formula (al′) covalently attached to a Camptothecin Drug Unit, wherein R¹ is hydrogen and R is dimethylaminoethyl. The phenyl nitro group is then reduced to an amine in order to provide a handle for sequential introduction of a Connector Unit (A) and a Stretcher Unit precursor (Z′).

### EXAMPLES

### Materials and Methods

The following materials and methods are applicable to the synthetic procedures described in this section unless indicated otherwise. All commercially available anhydrous solvents were used without further purification. Starting materials, reagents and solvents were purchased from commercial suppliers (SigmaAldrich and Fischer). Products were purified by flash column chromatography utilizing a Biotage Isolera One flash purification system (Charlotte, NC). UPLC-MS was performed on a Waters single quad detector mass spectrometer interfaced to a Waters Acquity UPLC system. UPLC methods are described below. Preparative HPLC was carried out on a Waters 2454 Binary Gradient Module solvent delivery system configured with a Wasters 2998 PDA detector or Teledyne ISCO ACCQPrep HP150. Products were purified with the appropriate diameter of column of a Phenomenex Max-RP 4 μm Synergi 80 A 250 mm reverse phase column eluting with 0.05% trifluoroacetic acid in water and 0.05% trifluoroacetic acid in acetonitrile unless otherwise specified.

General Method:

Column—Waters CORTECS C18 1.6 μm, 2.1×50 mm, reversed-phase column

Solvent A—0.1% aqueous formic acid

| Time (min) | Flow (mL/min) | A % | B % | Gradient |
|---|---|---|---|---|
| Initial | 0.6 | 97 | 3 | |
| 1.70 | 0.6 | 40 | 60 | Linear |
| 2.00 | 0.6 | 5 | 95 | Linear |
| 2.50 | 0.6 | 5 | 95 | Linear |
| 2.80 | 0.6 | 97 | 3 | Linear |
| 3.00 | 0.6 | 97 | 3 | Linear |
| 2.80 | 0.6 | 97 | 3 | Linear |

### LIST OF ABBREVIATIONS

| | |
|---|---|
| AcOH | acetic acid |
| Boc | tert-butyloxycarbonyl protecting group |
| DCM | dichloromethane |
| DIPEA | N,N-diisopropylethylamine |
| DMA | N,N-dimethyacetamide |
| DMF | N,N-dimethylformamide |
| EtOAc | ethyl acetate |
| EtOH | ethanol |
| Fmoc | 9-fluorenylmethyl carbamate |
| HATU | 1-[bis(dimethylamino)methylene]-1H-1,2,3-triazolo[4,5- |
| | b]pyridinium 3-oxid hexafluorophosphate |
| Hex | hexanes |
| HPLC | high performance liquid chromatography |
| MeCN | acetonitrile |
| MeOH | methanol |
| MP | 3-maleimidopropyl |
| MS | Mass spectrometry |
| OSu | N-hydroxysuccinimide |
| PEG | polyethylene glycol |
| PPTS | pyridinium para-toluene sulfonic acid |
| pTSA | para-toluene sulfonic acid |
| Prep | preparative |
| TFA | trifluoroacetic acid |
| TSTU | N,N,N′,N′-tetramethyl-O-(N-succinimidyl)uronium |
| | tetrafluoroborate |
| UPLC | Ultra Performance Liquid Chromatography |

### Experimental Methods

### Example 1

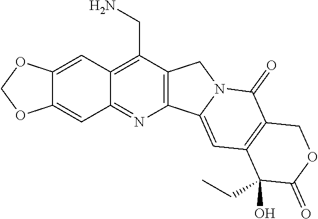 [SMILES: CC[C@@]1(O)C(=O)OCc2c1cc1n(c2=O)Cc2c-1nc1cc3c(cc1c2CN)OCO3]

Preparation of compound 1 was described in patent WO 2019195665

### Example 1a

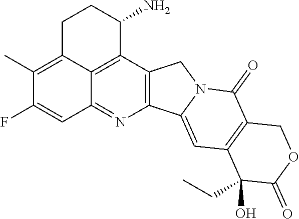 [SMILES: CC[C@@]1(O)C(=O)OCc2c1cc1n(c2=O)Cc2c-1nc1cc(F)c(C)c3c1c2[C@@H](N)CC3]

Compound 1a (Exatecan) was purchased from Advanced ChemBlock (Catalog #10484).

### Example 2: Preparation of Compound 2ao

### Step 1:

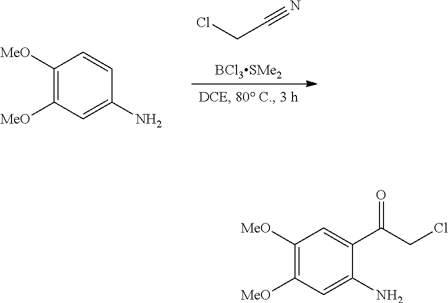

Boron trichloride methyl suflide complex (2M in DCM, 1.10 eq, 18 mL, 35.9 mmol) was diluted in DCE (110 mL) and the mixture was cooled to 0° C. under nitrogen atmosphere. 3,4-dimethoxyaniline (1.00 eq, 5000 mg, 32.6 mmol) diluted in DCE (20 mL) was added dropwise. The resulting solution was stirred for 15 minutes, and 2-chloroacetonitrile (1.10 eq, 2.3 mL, 35.9 mmol) was added. The reaction was warmed to room temperature, stirred for 15 minutes, and then heated at reflux for 3 hours. Nearly complete conversion to the imine/ketone intermediate was observed by UPLC-MS. The reaction was cooled to room temperature and 2M HCl (130 mL) was added. The reaction was heated to reflux for 30 minutes, then cooled to room temperature, and poured into ice water (150 mL). The aqueous was extracted with DCM (3×250 mL) and the combine organic extracts were washed with water (3×500 mL). Organic phase was dried with MgSO4, filtered and concentrated in vacuo. The crude material was purified by FCC 0-50% MeCN in DCM. Fractions containing the desired product were concentrated in vacuo to afford a tan solid 1-(2-amino-4,5-dimethoxy-phenyl)-2-chloro-ethanone (1573 mg, 6.85 mmol, 20.98% yield). Rt=1.25 min General Method UPLC. MS (m/z) [M+H]⁺ calc. for C₁₀H₁₃C₁NO₃ 230.06, found 230.28.

### Step 2:

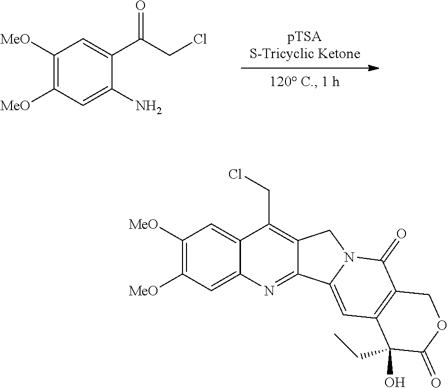 [SMILES: COc1cc(N)c(C(=O)CCl)cc1OC ; CC[C@@]1(O)C(=O)OCc2c1cc1n(c2=O)Cc2c-1nc1cc(OC)c(OC)cc1c2CCl]

(2-amino-4,5-dimethoxy-phenyl)-2-chloro-ethanone (1.00 eq, 200 mg, 0.871 mmol), Para-toluenesulfonic acid (1.00 eq, 150.0 mg, 0.871 mmol) and (45)-4-ethyl-4-hydroxy-7,8-dihydro-1 H-pyrano[3,4-f]indolizine-3,6,10-trione (1.10 eq, 252 mg, 0.958 mmol) were charged in a flask. DCM (2 mL) was added to homogenize the solids, and then evaporated under nitrogen. The neat solids were heated to 120° C. under high vacuum (1 mbar) for 60 minutes. The reaction was cooled to room temperature, the crude product was precipitated with water, filtered, washed with water and dried under high vacuum to afford a brown solid (257 mg, 0.563 mmol, 64.67% yield), which was used in the next step without further purification. Rt=1.35 min General Method UPLC. MS (m/z) [M+H]⁺ calc. for C₂₃H₂₂C₁N₂O₆ 457.12, found 457.56.

### Step 3:

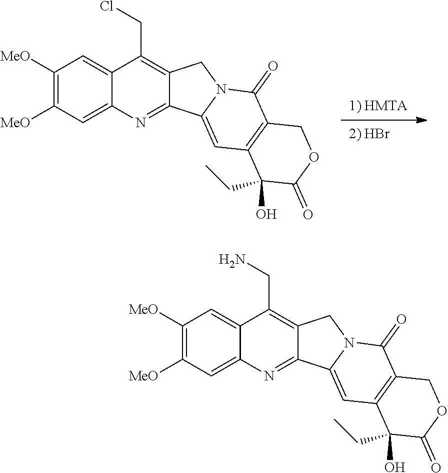 [SMILES: CC[C@@]1(O)C(=O)OCc2c1cc1n(c2=O)Cc2c-1nc1cc(OC)c(OC)cc1c2CCl ; CC[C@@]1(O)C(=O)OCc2c1cc1n(c2=O)Cc2c-1nc1cc(OC)c(OC)cc1c2CN]

(19S)-10-(chloromethyl)-19-ethyl-19-hydroxy-6,7-dimethoxy-17-oxa-3,13-diazapentacyclo[11.8.0.02,11.04,9.015,20]henicosa-1(21), 2,4(9),5,7,10,15(20)-heptaene-14,18-dione (1.00 eq, 257 mg, 0.563 mmol) was dissolved in Ethanol (4 mL). 1,3,5,7-tetrazatricyclo[3.3.1.13,7]decane (3.00 eq, 237 mg, 1.69 mmol) was added and the reaction was stirred at 65° C. for 24 hours. The reaction was quenched with a mixture of EtOH (4 mL) and 48% w/w aqueous HBr (0.8 mL). The reaction was stirred at 65° C. for 1 hour. The reaction was cooled to room temperature and concentrated in vacuo. The crude product was purified by prep-HPLC using a Synergi Max-RP 30×250 mm column eluting with MeCN in water 0.05% TFA. Fractions containing the desired product were concentrated in vacuo to afford a yellow solid (19S)-10-(aminomethyl)-19-ethyl-19-hydroxy-6,7-dimethoxy-17-oxa-3,13-diazapentacyclo[11.8.0.02,11.04,9.015,20]henicosa-1(21),2,4(9),5,7,10,15(20)-heptaene-14,18-dione (Compound 2ao) (32 mg,0.0722 mmol, 12.84%% yield). Rt=0.84 min General Method UPLC. MS (m/z) [M+H]⁺ calc. for C₂₃H₂₄N₃O₆ 438.17, found 438.35.

TABLE 2
Compounds listed below were made following the general procedures
outlined for compound 2ao.

| | | | Calc. | | Rt |
|---|---|---|---|---|---|
| No. | Structure | Exact Mass | [M + H]+ | m/z | (min) |
| 2a | 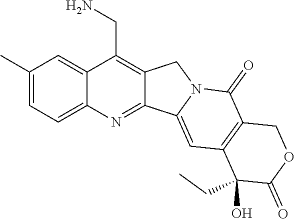 [SMILES: CC[C@@]1(O)C(=O)OCc2c1cc1n(c2=O)Cc2c-1nc1ccc(C)cc1c2CN] | 391.15 | 392.16 | 392.37 | 0.87 |
| 2b | 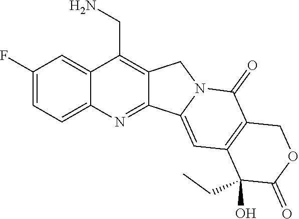 [SMILES: CC[C@@]1(O)C(=O)OCc2c1cc1n(c2=O)Cc2c-1nc1ccc(F)cc1c2CN] | 395.13 | 396.14 | 396.93 | 0.82 |
| 2c | 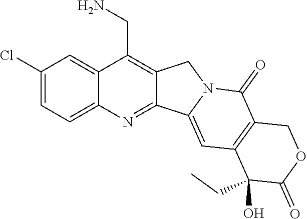 [SMILES: CC[C@@]1(O)C(=O)OCc2c1cc1n(c2=O)Cc2c-1nc1ccc(Cl)cc1c2CN] | 411.1 | 412.11 | 412.26 | 0.91 |
| 2d | 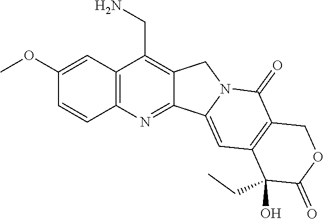 [SMILES: CC[C@@]1(O)C(=O)OCc2c1cc1n(c2=O)Cc2c-1nc1ccc(OC)cc1c2CN] | 407.15 | 408.16 | 408.67 | 0.89 |
| 2e | 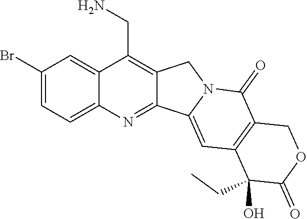 [SMILES: CC[C@@]1(O)C(=O)OCc2c1cc1n(c2=O)Cc2c-1nc1ccc(Br)cc1c2CN] | 455.05 | 456.06 | 456.4 | 0.98 |
| 2f | 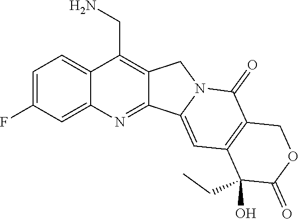 [SMILES: CC[C@@]1(O)C(=O)OCc2c1cc1n(c2=O)Cc2c-1nc1cc(F)ccc1c2CN] | 395.13 | 396.14 | 396.64 | 0.9 |
| 2g | 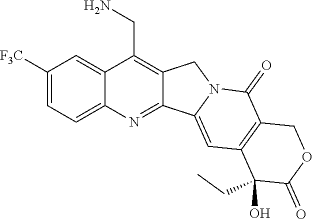 [SMILES: CC[C@@]1(O)C(=O)OCc2c1cc1n(c2=O)Cc2c-1nc1ccc(C(F)(F)F)cc1c2CN] | 445.12 | 446.13 | 446.99 | 1 |
| 2h | 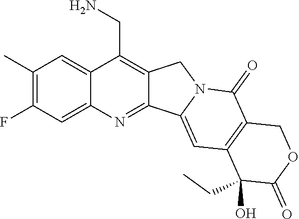 [SMILES: CC[C@@]1(O)C(=O)OCc2c1cc1n(c2=O)Cc2c-1nc1cc(F)c(C)cc1c2CN] | 409.14 | 410.15 | 410.32 | 1.03 |
| 2i | 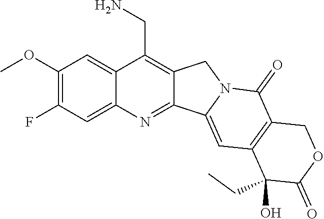 [SMILES: CC[C@@]1(O)C(=O)OCc2c1cc1n(c2=O)Cc2c-1nc1cc(F)c(OC)cc1c2CN] | 425.14 | 426.15 | 426.38 | 0.98 |
| 2j | 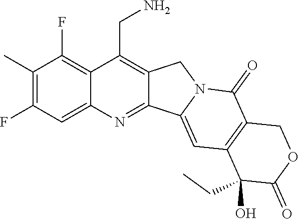 [SMILES: CC[C@@]1(O)C(=O)OCc2c1cc1n(c2=O)Cc2c-1nc1cc(F)c(C)c(F)c1c2CN] | 427.13 | 428.14 | 428.46 | 0.98 |
| 2k | 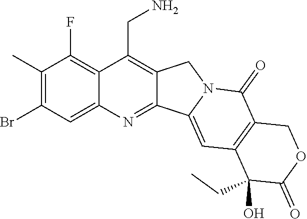 [SMILES: CC[C@@]1(O)C(=O)OCc2c1cc1n(c2=O)Cc2c-1nc1cc(Br)c(C)c(F)c1c2CN] | 487.05 | 488.06 | 488.6 | 1.15 |
| 2l | 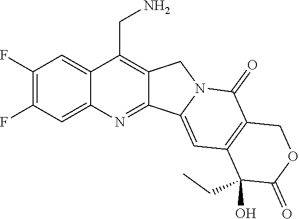 [SMILES: CC[C@@]1(O)C(=O)OCc2c1cc1n(c2=O)Cc2c-1nc1cc(F)c(F)cc1c2CN] | 413.12 | 414.13 | 414.53 | 0.95 |
| 2m | 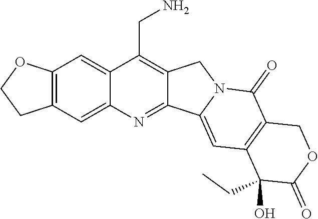 [SMILES: CC[C@@]1(O)C(=O)OCc2c1cc1n(c2=O)Cc2c-1nc1cc3c(cc1c2CN)OCC3] | 419.15 | 420.16 | 420.6 | 0.83 |
| 2n | 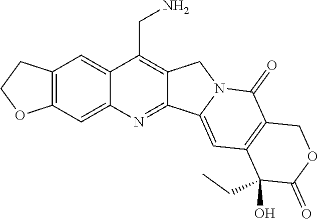 [SMILES: CC[C@@]1(O)C(=O)OCc2c1cc1n(c2=O)Cc2c-1nc1cc3c(cc1c2CN)CCO3] | 419.15 | 420.16 | 420.6 | 0.88 |
| 2o | 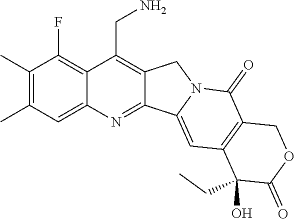 [SMILES: CC[C@@]1(O)C(=O)OCc2c1cc1n(c2=O)Cc2c-1nc1cc(C)c(C)c(F)c1c2CN] | 423.16 | 424.17 | 424.58 | 0.95 |
| 2p | 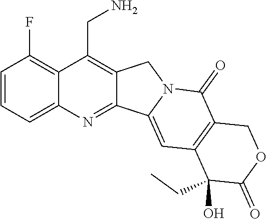 [SMILES: CC[C@@]1(O)C(=O)OCc2c1cc1n(c2=O)Cc2c-1nc1cccc(F)c1c2CN] | 395.13 | 396.14 | 396.25 | 0.82 |
| 2q | 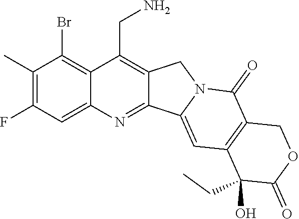 [SMILES: CC[C@@]1(O)C(=O)OCc2c1cc1n(c2=O)Cc2c-1nc1cc(F)c(C)c(Br)c1c2CN] | 487.05 | 488.06 | 488.57 | 1.11 |
| 2r | 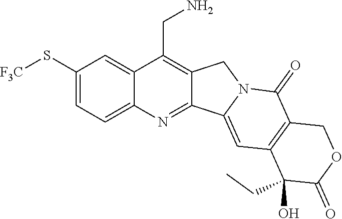 [SMILES: CC[C@@]1(O)C(=O)OCc2c1cc1n(c2=O)Cc2c-1nc1ccc(SC(F)(F)F)cc1c2CN] | 477.1 | 478.11 | 478.32 | 1.13 |
| 2s | 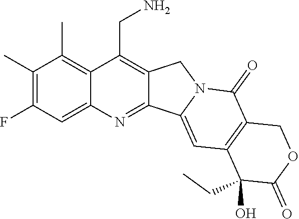 [SMILES: CC[C@@]1(O)C(=O)OCc2c1cc1n(c2=O)Cc2c-1nc1cc(F)c(C)c(C)c1c2CN] | 423.16 | 424.17 | 424.68 | 1.04 |
| 2t | 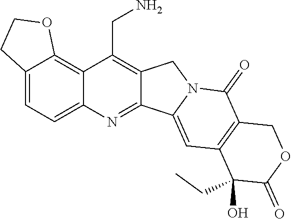 [SMILES: CC[C@@]1(O)C(=O)OCc2c1cc1n(c2=O)Cc2c-1nc1ccc3c(c1c2CN)OCC3] | 419.15 | 420.16 | 420.6 | 0.9 |
| 2u | 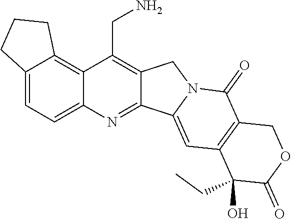 [SMILES: CC[C@@]1(O)C(=O)OCc2c1cc1n(c2=O)Cc2c-1nc1ccc3c(c1c2CN)CCC3] | 417.17 | 418.18 | 418.66 | 0.97 |
| 2v | 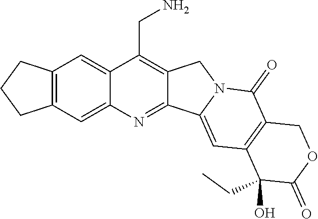 [SMILES: CC[C@@]1(O)C(=O)OCc2c1cc1n(c2=O)Cc2c-1nc1cc3c(cc1c2CN)CCC3] | 417.17 | 418.18 | 418.56 | 0.99 |
| 2w | 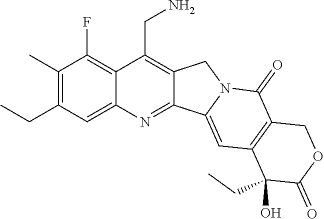 [SMILES: CCc1cc2nc3c(c(CN)c2c(F)c1C)Cn1c-3cc2c(c1=O)COC(=O)[C@]2(O)CC] | 437.18 | 438.19 | 438.64 | 1.06 |
| 2x | 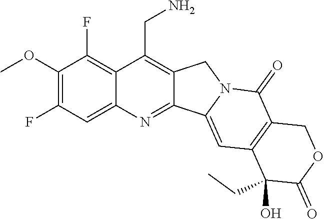 [SMILES: CC[C@@]1(O)C(=O)OCc2c1cc1n(c2=O)Cc2c-1nc1cc(F)c(OC)c(F)c1c2CN] | 443.13 | 444.14 | 444.46 | 0.92 |
| 2y | 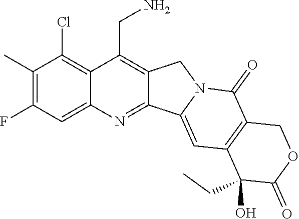 [SMILES: CC[C@@]1(O)C(=O)OCc2c1cc1n(c2=O)Cc2c-1nc1cc(F)c(C)c(Cl)c1c2CN] | 443.1 | 444.11 | 444.46 | 1.01 |
| 2z | 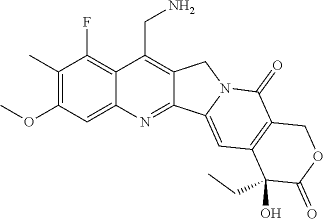 [SMILES: CC[C@@]1(O)C(=O)OCc2c1cc1n(c2=O)Cc2c-1nc1cc(OC)c(C)c(F)c1c2CN] | 439.15 | 440.16 | 440.54 | 1 |
| 2aa | 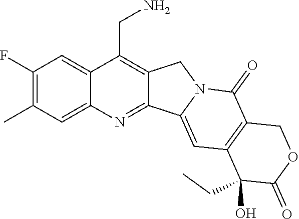 [SMILES: CC[C@@]1(O)C(=O)OCc2c1cc1n(c2=O)Cc2c-1nc1cc(C)c(F)cc1c2CN] | 409.14 | 410.15 | 410.22 | 0.97 |
| 2ab | 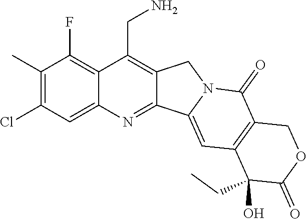 [SMILES: CC[C@@]1(O)C(=O)OCc2c1cc1n(c2=O)Cc2c-1nc1cc(Cl)c(C)c(F)c1c2CN] | 443.1 | 444.11 | 444.66 | 1.05 |
| 2ac | 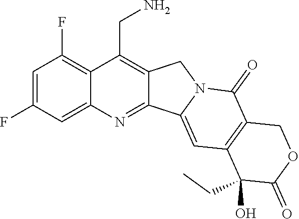 [SMILES: CC[C@@]1(O)C(=O)OCc2c1cc1n(c2=O)Cc2c-1nc1cc(F)cc(F)c1c2CN] | 413.12 | 414.13 | 414.59 | 0.84 |
| 2ad | 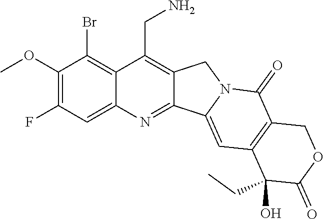 [SMILES: CC[C@@]1(O)C(=O)OCc2c1cc1n(c2=O)Cc2c-1nc1cc(F)c(OC)c(Br)c1c2CN] | 503.05 | 504.06 | 504.32 | 1.05 |
| 2ae | 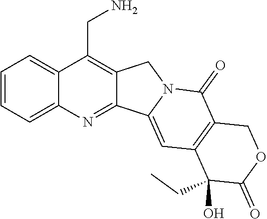 [SMILES: CC[C@@]1(O)C(=O)OCc2c1cc1n(c2=O)Cc2c-1nc1ccccc1c2CN] | 377.14 | 378.15 | 378.6 | 0.78 |
| 2af | 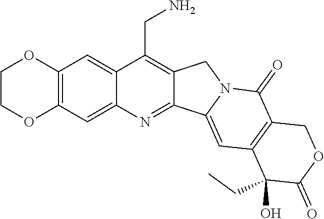 [SMILES: CC[C@@]1(O)C(=O)OCc2c1cc1n(c2=O)Cc2c-1nc1cc3c(cc1c2CN)OCCO3] | 435.14 | 436.15 | 436.61 | 0.88 |
| 2ag | 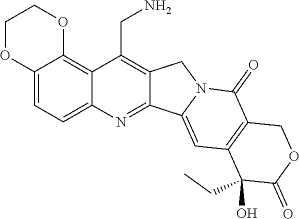 [SMILES: CC[C@@]1(O)C(=O)OCc2c1cc1n(c2=O)Cc2c-1nc1ccc3c(c1c2CN)OCCO3] | 435.14 | 436.15 | 436.51 | 0.9 |
| 2ah | 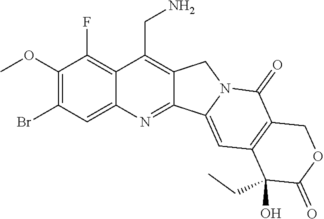 [SMILES: CC[C@@]1(O)C(=O)OCc2c1cc1n(c2=O)Cc2c-1nc1cc(Br)c(OC)c(F)c1c2CN] | 503.05 | 504.06 | 504.41 | 0.96 |
| 2ai | 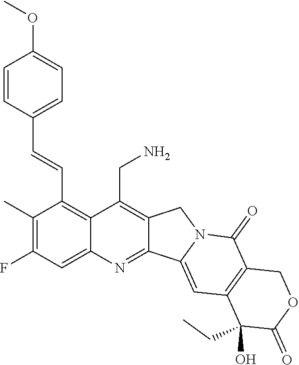 [SMILES: CC[C@@]1(O)C(=O)OCc2c1cc1n(c2=O)Cc2c-1nc1cc(F)c(C)c(/C=C/c3ccc(OC)cc3)c1c2CN] | 541.2 | 542.21 | 542.05 | 1.32 |
| 2aj | 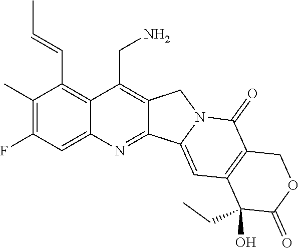 [SMILES: C/C=C/c1c(C)c(F)cc2nc3c(c(CN)c12)Cn1c-3cc2c(c1=O)COC(=O)[C@]2(O)CC] | 449.18 | 450.19 | 450.19 | 1.11 |
| 2ak | 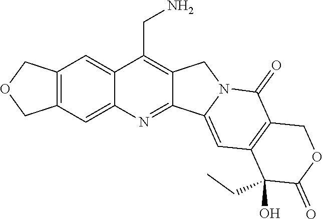 [SMILES: CC[C@@]1(O)C(=O)OCc2c1cc1n(c2=O)Cc2c-1nc1cc3c(cc1c2CN)COC3] | 419.15 | 420.16 | 420.60 | 0.79 |
| 2al | 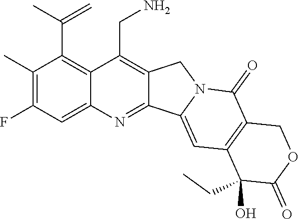 [SMILES: C=C(C)c1c(C)c(F)cc2nc3c(c(CN)c12)Cn1c-3cc2c(c1=O)COC(=O)[C@]2(O)CC] | 449.18 | 450.19 | 450.38 | 1.06 |
| 2am | 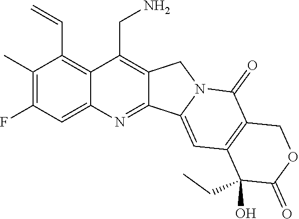 [SMILES: C=Cc1c(C)c(F)cc2nc3c(c(CN)c12)Cn1c-3cc2c(c1=O)COC(=O)[C@]2(O)CC] | 435.16 | 436.17 | 436.22 | 1.03 |
| 2an | 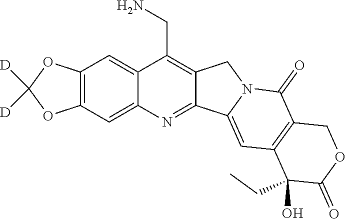 [SMILES: [2H]C1([2H])Oc2cc3nc4c(c(CN)c3cc2O1)Cn1c-4cc2c(c1=O)COC(=O)[C@]2(O)CC] | 423.14 | 424.15 | 424.48 | 0.86 |
| 2ap | 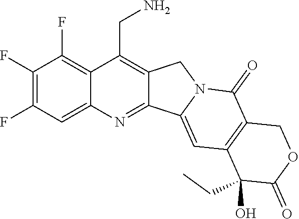 [SMILES: CC[C@@]1(O)C(=O)OCc2c1cc1n(c2=O)Cc2c-1nc1cc(F)c(F)c(F)c1c2CN] | 431.11 | 432.12 | 432.05 | 0.99 |
| 2aq | 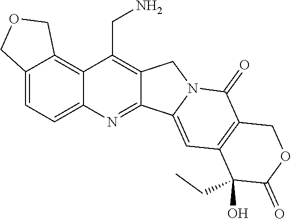 [SMILES: CC[C@@]1(O)C(=O)OCc2c1cc1n(c2=O)Cc2c-1nc1ccc3c(c1c2CN)COC3] | 419.15 | 420.16 | 420.47 | 1.00 |
| 2ar | 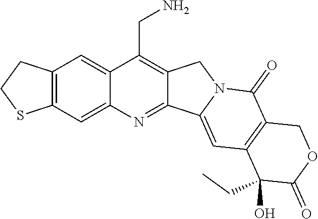 [SMILES: CC[C@@]1(O)C(=O)OCc2c1cc1n(c2=O)Cc2c-1nc1cc3c(cc1c2CN)CCS3] | 435.13 | 436.14 | 436.32 | 1.06 |
| 2as | 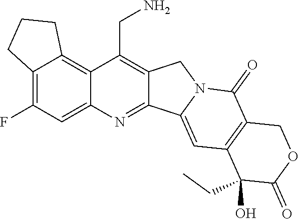 [SMILES: CC[C@@]1(O)C(=O)OCc2c1cc1n(c2=O)Cc2c-1nc1cc(F)c3c(c1c2CN)CCC3] | 435.16 | 436.17 | 435.84 | 1.09 |
| 2at | 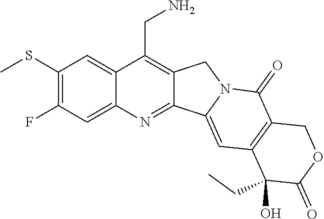 [SMILES: CC[C@@]1(O)C(=O)OCc2c1cc1n(c2=O)Cc2c-1nc1cc(F)c(SC)cc1c2CN] | 441.12 | 442.13 | 442.20 | 1.04 |
| 2au | 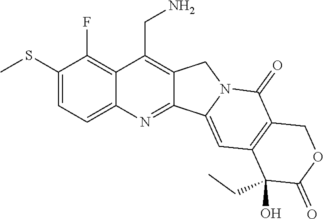 [SMILES: CC[C@@]1(O)C(=O)OCc2c1cc1n(c2=O)Cc2c-1nc1ccc(SC)c(F)c1c2CN] | 441.12 | 442.13 | 442.11 | 1.16 |
| 2av | 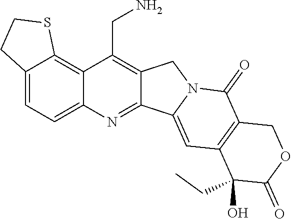 [SMILES: CC[C@@]1(O)C(=O)OCc2c1cc1n(c2=O)Cc2c-1nc1ccc3c(c1c2CN)SCC3] | 435.13 | 436.14 | 436.41 | 1.13 |
| 2aw | 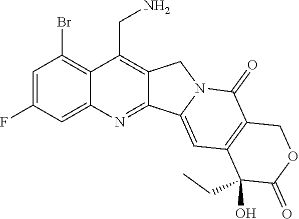 [SMILES: CC[C@@]1(O)C(=O)OCc2c1cc1n(c2=O)Cc2c-1nc1cc(F)cc(Br)c1c2CN] | 473.04 | 474.05 | 475.11 | 1.09 |
| 2ax | 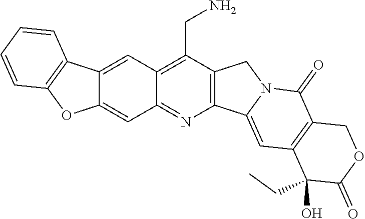 [SMILES: CC[C@@]1(O)C(=O)OCc2c1cc1n(c2=O)Cc2c-1nc1cc3oc4ccccc4c3cc1c2CN] | 467.15 | 468.16 | 468.48 | 1.17 |
| 2ay | 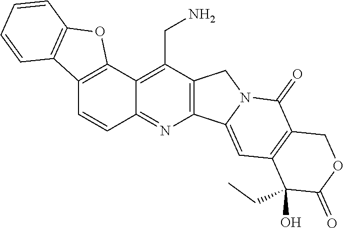 [SMILES: CC[C@@]1(O)C(=O)OCc2c1cc1n(c2=O)Cc2c-1nc1ccc3c4ccccc4oc3c1c2CN] | 467.15 | 468.16 | 468.33 | 1.12 |
| 2az | 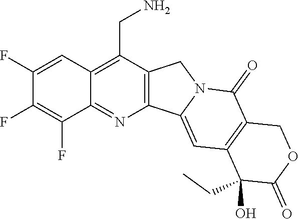 [SMILES: CC[C@@]1(O)C(=O)OCc2c1cc1n(c2=O)Cc2c-1nc1c(F)c(F)c(F)cc1c2CN] | 431.11 | 432.12 | 432.33 | 0.72 |
| 2aaa | 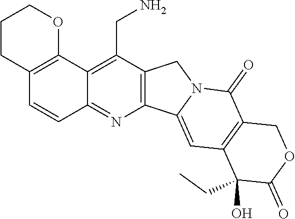 [SMILES: CC[C@@]1(O)C(=O)OCc2c1cc1n(c2=O)Cc2c-1nc1ccc3c(c1c2CN)OCCC3] | 433.16 | 434.17 | 434.33 | 0.74 |
| 2aab | 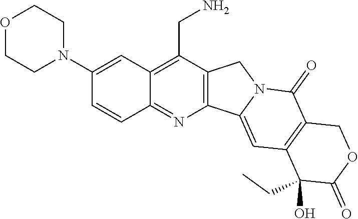 [SMILES: CC[C@@]1(O)C(=O)OCc2c1cc1n(c2=O)Cc2c-1nc1ccc(N3CCOCC3)cc1c2CN] | 462.19 | 463.20 | 463.52 | 0.71 |
| 2aac | 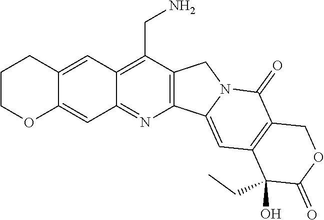 [SMILES: CC[C@@]1(O)C(=O)OCc2c1cc1n(c2=O)Cc2c-1nc1cc3c(cc1c2CN)CCCO3] | 433.16 | 434.17 | 434.33 | 0.73 |

### Example 3: Preparation of Compound 3e

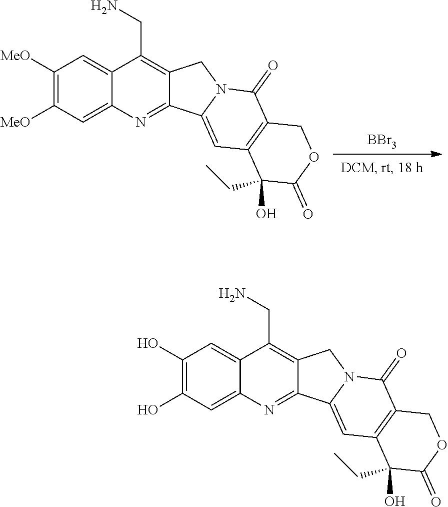

rac-(19S)-10-(aminomethyl)-19-ethyl-19-hydroxy-6,7-dimethoxy-17-oxa-3,13-diazapentacyclo[11.8.0.02,11.04,9.015,20]henicosa-1(21),2,4,6,8,10,15(20)-heptaene-14,18-dione (1.00 eq, 26 mg, 0.0594 mmol) was dissolved in DCM (0.5944 mL). Boron tribromide (10.0 eq, 0.59 mL, 0.594 mmol) was added and the reaction was stirred for 15 hours. The reaction was diluted into a stirred mixture of methanol (20 mL) and concentrated in vacuo. The crude product was purified by prep-HPLC using a Synergi Max-RP 21.2×250 column eluting with MeCN in water 0.05% TFA. Fractions containing the desired product were concentrated in vacuo to afford a yellow solid (19S)-10-(aminomethyl)-19-ethyl-6,7,19-trihydroxy-17-oxa-3,13-diazapentacyclo[11.8.0.02,11.04,9.015,20]henicosa-1(21),2,4(9),5,7,10,15(20)-heptaene-14,18-dione Compound 3e (2.1 mg, 0.00520 mmol, 8.75% yield). Rt=0.69 min General Method UPLC. MS (m/z) [M+H]⁺ calc. for C₂₁H₂₀N₃O₆ 410.14, found 410.61.

TABLE 3
Compounds 3a-3d were made following the general procedures outlined
for Compound 3e.

| | | | Calc. | | Rt |
|---|---|---|---|---|---|
| No. | Structure | Exact Mass | [M + H]+ | m/z | (min) |
| 3a | 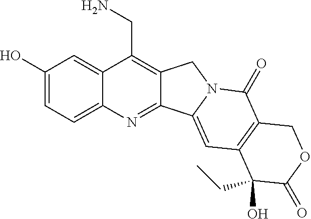 [SMILES: CC[C@@]1(O)C(=O)OCc2c1cc1n(c2=O)Cc2c-1nc1ccc(O)cc1c2CN] | 393.13 | 394.14 | 394.22 | 0.78 |
| 3b | 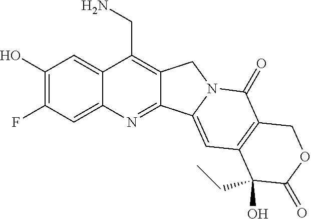 [SMILES: CC[C@@]1(O)C(=O)OCc2c1cc1n(c2=O)Cc2c-1nc1cc(F)c(O)cc1c2CN] | 411.12 | 412.13 | 412.26 | 0.8 |
| 3c | 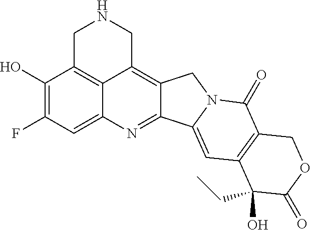 [SMILES: CC[C@@]1(O)C(=O)OCc2c1cc1n(c2=O)Cc2c-1nc1cc(F)c(O)c3c1c2CNC3] | 423.12 | 424.13 | 424.58 | 0.76 |
| 3d | 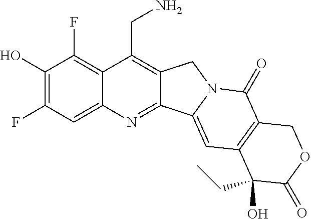 [SMILES: CC[C@@]1(O)C(=O)OCc2c1cc1n(c2=O)Cc2c-1nc1cc(F)c(O)c(F)c1c2CN] | 429.11 | 430.12 | 430.01 | 0.82 |

### Example 4. Preparation of Compound 4

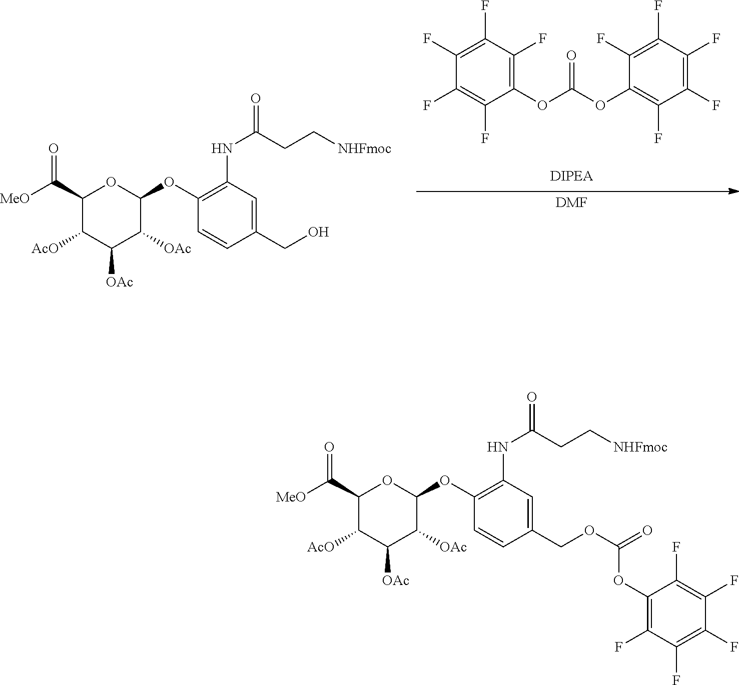

methyl rac-(2S,3S,4S,5R,6S)-3,4,5-triacetoxy-6-[2-[3-(9H-fluoren-9-ylmethoxycarbonylamino)propanoylamino]-4-(hydroxymethyl)phenoxy]tetrahydropyran-2-carboxylate (1.00 eq, 2000 mg, 2.67 mmol), prepared according to the procedure of Bioconjugate Chem. (2006) 17: 831-840), was dissolved in DMF (8.904 mL). Bis(pentafluorophenyl) carbonate (1.50 eq, 1579 mg, 4.01 mmol) was added to the reaction followed by N,N-Diisopropylethylamine (2.00 eq, 0.93 mL, 5.34 mmol). The reaction was stirred for 30 minutes at which point complete conversion was observed by UPLC-MS. The reaction was diluted with EtOAC (100 mL), washed with 13% NaCl (2×100 mL), washed with brine, dried MgSO4, filtered and concentrated in vacuo. The crude product was purified by FCC 10-100% EtOAC in Hex. Fractions containing the desired product were concentrated in vacuo to afford a colorless solid methyl (2S,3S,4S,5R,6S)-3,4,5-triacetoxy-6-[2-[3-(9H-fluoren-9-ylmethoxycarbonylamino)propanoylamino]-4-[(2,3,4,5,6-pentafluorophenoxy)carbonyloxymethyl]phenoxy]tetrahydropyran-2-carboxylate (2.27 g, 2.37 mmol, 88.57% yield). Rt=2.26 min General Method UPLC. MS (m/z) [M+H]⁺ calc. for C₄₅H₄₀F₅N₂O₁₆ 959.23, found 959.32.

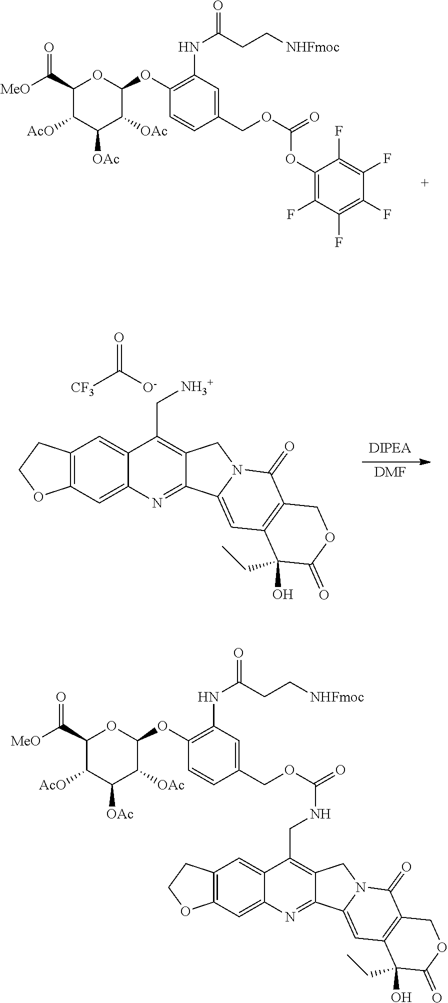

(5S)-14-(aminomethyl)-5-ethyl-5-hydroxy-7,20-dioxa-11,24-diazahexacyclo[11.11.0.02,11.04,9.015,23.017,21]tetracosa-1(24),2,4(9),13,15(23),16,21-heptaene-6,10-dione;2,2,2-trifluoroacetic acid Compound 2n (1.00 eq, 135 mg, 0.254 mmol) and methyl (2S,3S,4S,5R,6S)-3,4,5-triacetoxy-6-[2-[3-(9H-fluoren-9-ylmethoxycarbonylamino)propanoylamino]-4-[(2,3,4,5,6-pentafluorophenoxy)carbonyloxymethyl]phenoxy]tetrahydropyran-2-carboxylate (1.00 eq, 243 mg, 0.254 mmol) were dissolved in DMF (1 mL). N,N-Diisopropylethylamine (1.50 eq, 0.066 mL, 0.380 mmol) was added and the reaction was stirred for 20 minutes. Complete conversion was observed by UPLC-MS. The reaction was acidified with AcOH (100 uL), and concentrated in vacuo. The residue was purified by FCC 25G Sfar Silica HC-D 0-12% McOH in DCM. Fractions containing the desired product were concentrated in vacuo to afford a yellow amorphous solid methyl (25,35,45,5R,65)-3,4,5-triacetoxy-6-[4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]-2-[3-(9H-fluoren-9-ylmethoxycarbonylamino)propanoylamino]phenoxy]tetrahydropyran-2-carboxylate (203 mg, 0.170 mmol, 67.05% yield). Rt=2.04 min General Method UPLC. MS (m/z) [M+H]⁺ calc. for C₆₂H₆₀N₅O₂₀ 1194.38, found 1194.55.

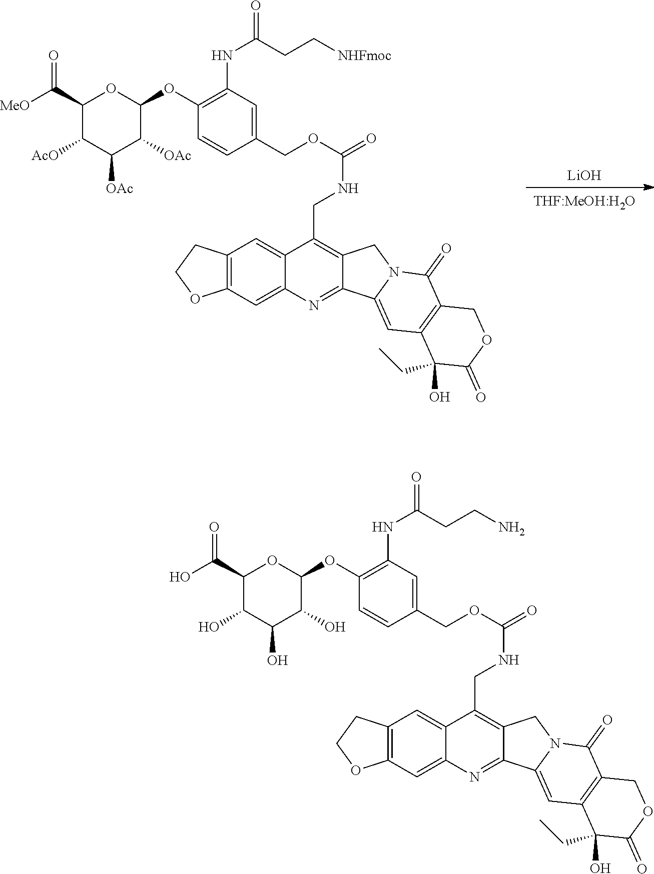

methyl (2S,3S,4S,5R,65)-3,4,5-triacetoxy-6-[4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethy1]-213-(9H-fluoren-9-ylmethoxycarbonylamino)propanoylamino]phenoxy]tetrahydropyran-2-carboxylate (1.00 eq, 203 mg, 0.170 mmol) was dissolved in Methanol (2 mL) and THE (2 mL). The reaction was cooled to OC with an ice/water bath then lithium;hydroxide (30.0 eq, 122 mg, 5.10 mmol) was added. The reaction was stirred for 10 minutes at which point complete acetate deprotection was observed. Water (2 mL) was added to the reaction and stirred for 10 minutes at which point complete deprotection was observed. Note: reversible lactone hydrolysis is observed (M+18). The reaction was acidified with AcOH (500 mL) and concentrated in vacuo. Purified by prep-HPLC 30×250 mm MaxRP 10-30-95% MeCN in H2O 0.05% TFA. Fractions containg the desired product were concentrated in vacuo to afford a yellow solid (2S,3S,4S,5R,6S)-6-[2-(3-aminopropanoylamino)-4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]phenoxy]-3,4,5-trihydroxy-tetrahydropyran-2-carboxylic acid (100 mg, 0.120 mmol, 70.51% yield). Rt=0.98 min General Method UPLC. MS (m/z) [M+H]⁺ calc. for C₄₀H₄₂N₅O₁₅ 832.27, found 832.42.

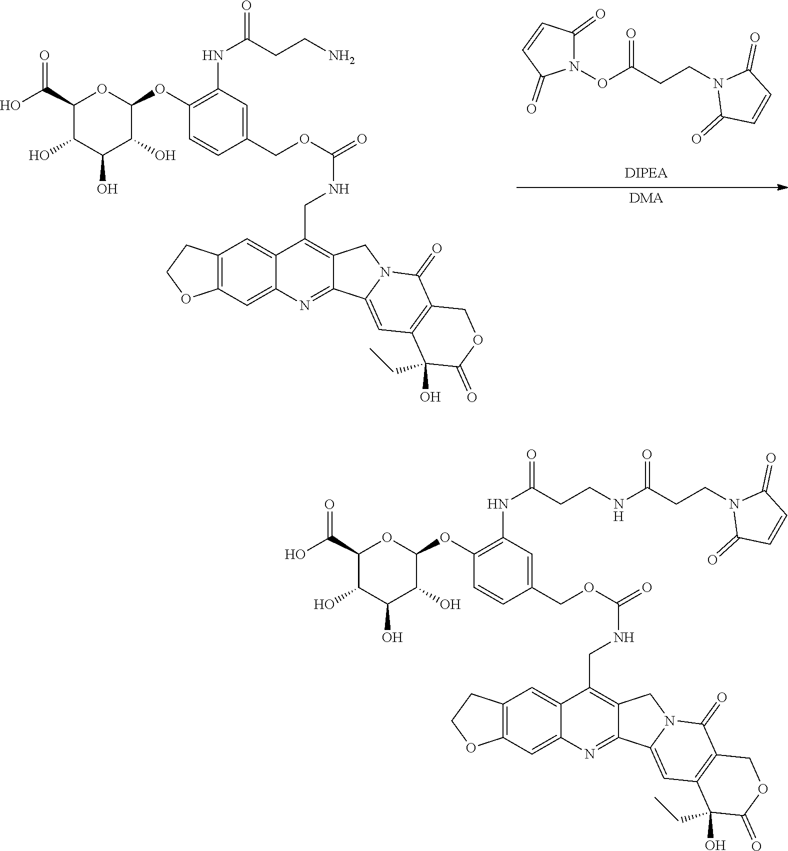

(2S,3S,4S,5R,6S)-6-[2-(3-aminopropanoylamino)-4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]phenoxy]-3,4,5-trihydroxy-tetrahydropyran-2-carboxylic acid (1.00 eq, 100 mg, 0.120 mmol) was dissolved in DMA (1 mL). 2,5-dioxopyrrolidin-1-yl 3-(2,5-dioxo-2,5-dihydro-1 H-pyirol-1-yl)propanoate (1.20 eq, 38 mg, 0.144 mmol) was added followed by N,N-Diisopropylethylamine (1.50 eq, 0.031 mL, 0.180 mmol). The reaction was stirred for 5 minutes at which point comlpete conversion was observed by UPLC-MS. The reaction was acidified with AcOH (50 uL) and purified by prep-HPLC 5-40-95% MeCN in H2O 0.05% TFA. Fractions containing the desired product were concentrated in vacuo to afford a yellow solid (2S,3S,4S,5R,6S)-6-[2-[3-[3-(2,5-dioxopyrrol-1-yl)propanoylamino]propanoylamino]-4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]phenoxy]-3,4,5-trihydroxy-tetrahydropyran-2-carboxylic acid Compound 4 (91 mg, 0.0931 mmol, 77.59% yield). Rt=1.11 min General Method UPLC. MS (m/z) [M+H]⁺ calc. for C₄₇H47N₆O₁₈ 983.30, found 983.30.

TABLE 4
Compounds listed below were made following the
general procedures outlined for Compound 4.

| | | | Camptothecin |
|---|---|---|---|
| No. | Z′—A | RL | (N-link) |
| 4 | Mal-CH₂CH₂C(O)—NHCH₂CH₂C(O)— | Glucuronide | Compound 2n |
| 4a | Mal-CH₂CH₂C(O)—NHCH₂CH₂C(O)— | Glucuronide | Compound 2u |
| 4b | Mal-CH₂CH₂C(O)—NHCH₂CH₂C(O)— | Glucuronide | Compound 2am |
| 4c | Mal-CH₂CH₂C(O)—NHCH₂CH₂C(O)— | Glucuronide | Compound 2al |
| 4d | Mal-CH₂CH₂C(O)—NHCH₂CH₂C(O)— | Glucuronide | Compound 2j |
| 4e | Mal-CH₂CH₂C(O)—NHCH₂CH₂C(O)— | Glucuronide | Compound 2k |
| 4f | Mal-CH₂CH₂C(O)—NHCH₂CH₂C(O)— | Glucuronide | Compound 1 |
| 4g | Mal-CH₂CH₂C(O)—NHCH₂CH₂C(O)— | Glucuronide | Compound 2i |
| 4h | Mal-CH₂CH₂C(O)—NHCH₂CH₂C(O)— | Glucuronide | Compound 2x |
| 4i | Mal-CH₂CH₂C(O)—NHCH₂CH₂C(O)— | Glucuronide | Compound 2ap |
| 4j | Mal-CH₂CH₂C(O)—NHCH₂CH₂C(O)— | Glucuronide | Compound 2ac |
| 4k | Mal-CH₂CH₂C(O)—NHCH₂CH₂C(O)— | Glucuronide | Compound 2as |
| 4l | Mal-CH₂CH₂C(O)—NHCH₂CH₂C(O)— | Glucuronide | Compound 2aq |
| 4m | Mal-CH₂CH₂C(O)—NHCH₂CH₂C(O)— | Glucuronide | Compound 6d |
| 4n | Mal-CH₂CH₂C(O)—NHCH₂CH₂C(O)— | Glucuronide | Compound 2ag |
| 4o | Mal-CH₂CH₂C(O)—NHCH₂CH₂C(O)— | Glucuronide | Compound 2h |
| 4p | Mal-CH₂CH₂C(O)—N(CH₃)CH₂C(O)— | MAC | Compound 15d |
| 4q | Mal-CH₂CH₂C(O)—N(CH₃)CH₂C(O)— | MAC | Compound 15c |
| 4r | Mal-CH₂CH₂C(O)—NHCH₂CH₂C(O)— | Glucuronide | Compound 6e |
| 4s | Mal-CH₂CH₂C(O)—NHCH₂CH₂C(O)— | Glucuronide | Compound 6g |
| 4t | Mal-CH₂CH₂C(O)—NHCH₂CH₂C(O)— | Glucuronide | Compound 6p |
| 4u | Mal-CH₂CH₂C(O)—NHCH₂CH₂C(O)— | Glucuronide | Compound 2q |
| 4v | Mal-CH₂CH₂C(O)—N(CH₃)CH₂C(O)— | MAC | Compound 15a |
| 4w | Mal-CH₂CH₂C(O)—N(CH₃)CH₂C(O)— | MAC | Compound 15b |
| 4x | Mal-CH2CH2CH₂CH₂CH₂C(O)—NHCH₂CH₂C(O)— | Glucuronide | Compound 2n |
| 4y | Mal-CH₂CH₂C(O)—NHCH₂CH₂C(O)— | Mannose | Compound 2n |
| 4z | Mal-CH₂CH₂C(O)-Lys(PEG12)-Val-Cit- | PABC | Compound 2n |
| 4aa | Mal-CH₂CH₂C(O)—NHCH₂CH₂C(O)— | Galactose | Compound 2n |
| 4ab | Mal-CH₂CH₂C(O)— | | Compound 2n |

Characterization Data

| Compound | Parent Exact | Calc'd MS | Observed MS | |
|---|---|---|---|---|
| No. | Mass | (m/z) [M + H]⁺ | (m/z) | RT |
| 4 | 982.29 | 983.30 | 983.30 | 1.11 |
| 4a | 980.31 | 981.32 | 981.96 | 1.48 |
| 4b | 998.30 | 999.31 | 999.80 | 1.56 |
| 4c | 1012.30 | 1013.31 | 1013.57 | 1.40 |
| 4d | 990.27 | 991.28 | 991.64 | 1.45 |
| 4e | 1050.20 | 1051.21 | 1051.63 | 1.59 |
| 4f | 984.27 | 985.28 | 985.45 | 1.24 |
| 4g | 988.28 | 989.29 | 989.28 | 1.28 |
| 4h | 1006.27 | 1007.28 | 1007.72 | 1.35 |
| 4i | 994.25 | 995.26 | 995.39 | 1.27 |
| 4j | 976.26 | 977.27 | 977.62 | 1.31 |
| 4k | 998.30 | 999.31 | 999.75 | 1.45 |
| 4l | 982.29 | 983.29 | 983.27 | 1.21 |
| 4m | 1040.31 | 1041.32 | 1041.35 | 1.40 |
| 4n | 1042.30 | 1043.31 | 1043.80 | 1.35 |
| 4o | 972.28 | 973.29 | 972.97 | 1.42 |
| 4p | 1177.31 | 1178.32 | 1178.87 | 1.29 |
| 4q | 1175.33 | 1176.34 | 1176.81 | 1.31 |
| 4r | 1084.28 | 1085.29 | 1085.86 | 1.46 |
| 4s | 1054.27 | 1055.28 | 1055.09 | 1.63 |
| 4t | 1038.29 | 1039.30 | 1039.01 | 1.58 |
| 4u | 1050.19 | 1051.20 | 1051.04 | 1.55 |
| 4v | 1191.34 | 1192.35 | 1191.96 | 1.45 |
| 4w | 1205.35 | 1206.36 | 1206.98 | 1.50 |
| 4x | 1024.33 | 1025.34 | 1025.13 | 1.38 |
| 4y | 968.31 | 969.32 | 969.19 | 1.28 |
| 4z | 1675.80 | 1675.81 | 1675.73 | 1.51 |
| 4aa | 968.31 | 969.32 | 969.12 | 1.26 |
| 4ab | 570.18 | 571.19 | 570.95 | 1.33 |

| No. | Structure |
|---|---|
| 4 | 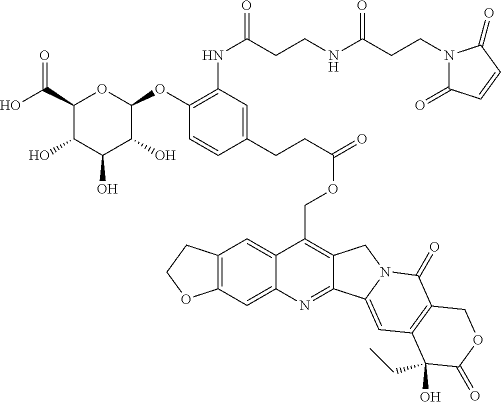 [SMILES: CC[C@@]1(O)C(=O)OCc2c1cc1n(c2=O)Cc2c-1nc1cc3c(cc1c2COC(=O)CCc1ccc(O[C@@H]2O[C@H](C(=O)O)[C@@H](O)[C@H](O)[C@H]2O)c(NC(=O)CCNC(=O)CCN2C(=O)C=CC2=O)c1)CCO3] |
| 4a | 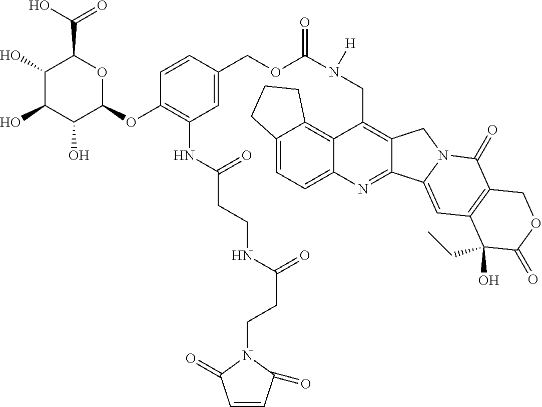 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCC3] |
| 4b | 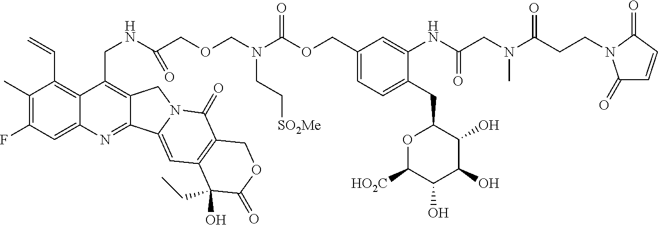 [SMILES: C=Cc1c(C)c(F)cc2nc3c(c(CNC(=O)COCN(CCS(C)(=O)=O)C(=O)OCc4ccc(C[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4c | 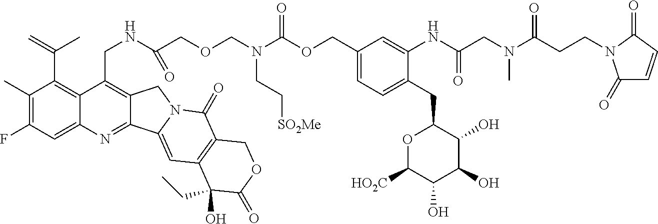 [SMILES: C=C(C)c1c(C)c(F)cc2nc3c(c(CNC(=O)COCN(CCS(C)(=O)=O)C(=O)OCc4ccc(C[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4d | 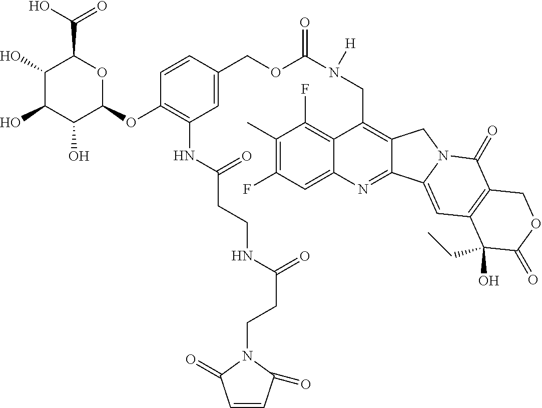 [SMILES: CC[C@@]1(O)C(=O)OCc2c1cc1n(c2=O)Cc2c-1nc1cc(F)c(C)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4e | 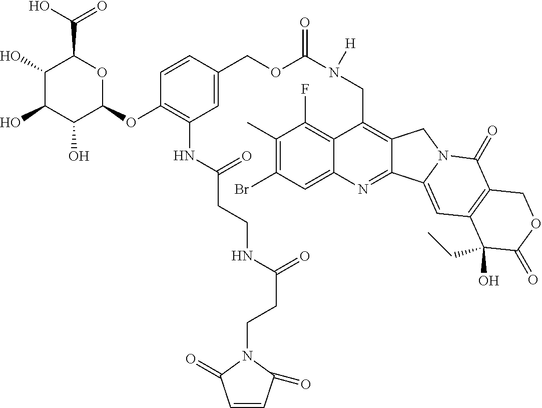 [SMILES: CC[C@@]1(O)C(=O)OCc2c1cc1n(c2=O)Cc2c-1nc1cc(Br)c(C)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4f | 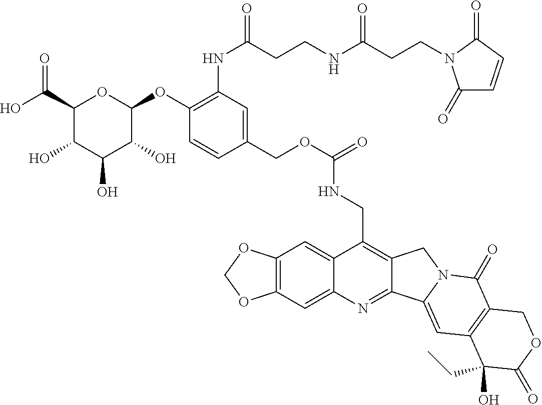 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCO3] |
| 4g | 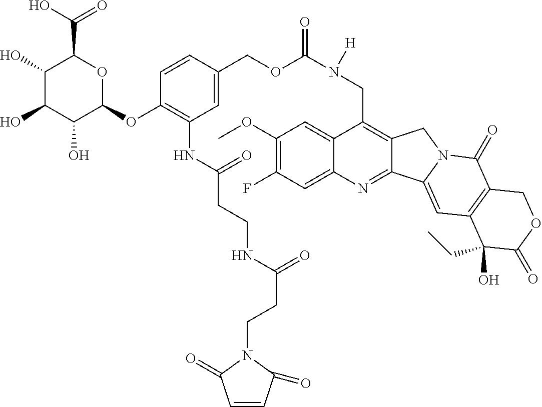 [SMILES: CC[C@@]1(O)C(=O)OCc2c1cc1n(c2=O)Cc2c-1nc1cc(F)c(OC)cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4h | 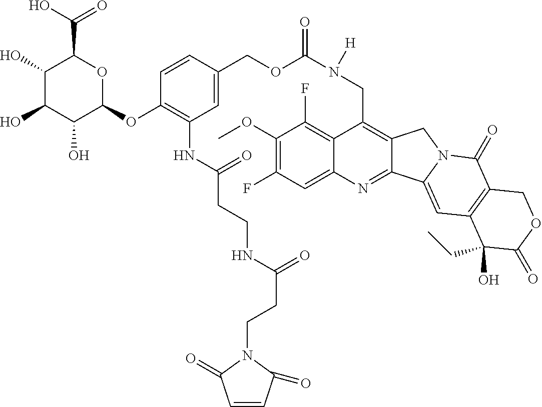 [SMILES: CC[C@@]1(O)C(=O)OCc2c1cc1n(c2=O)Cc2c-1nc1cc(F)c(OC)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4i | 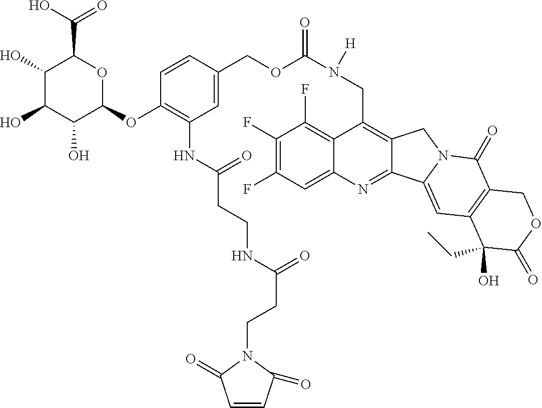 [SMILES: CC[C@@]1(O)C(=O)OCc2c1cc1n(c2=O)Cc2c-1nc1cc(F)c(F)c(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4j | 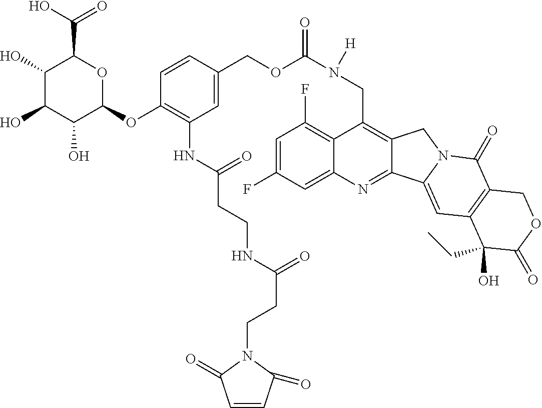 [SMILES: CC[C@@]1(O)C(=O)OCc2c1cc1n(c2=O)Cc2c-1nc1cc(F)cc(F)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4k | 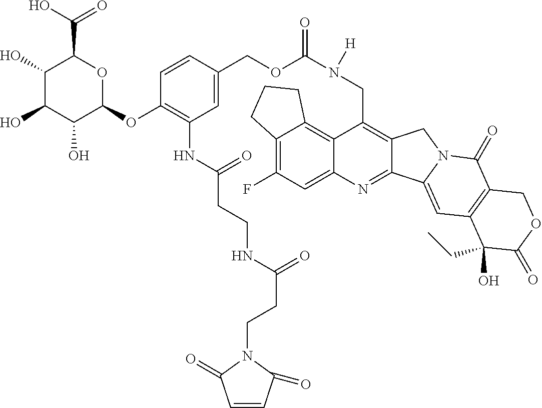 [SMILES: CC[C@@]1(O)C(=O)OCc2c1cc1n(c2=O)Cc2c-1nc1cc(F)c3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCC3] |
| 4l | 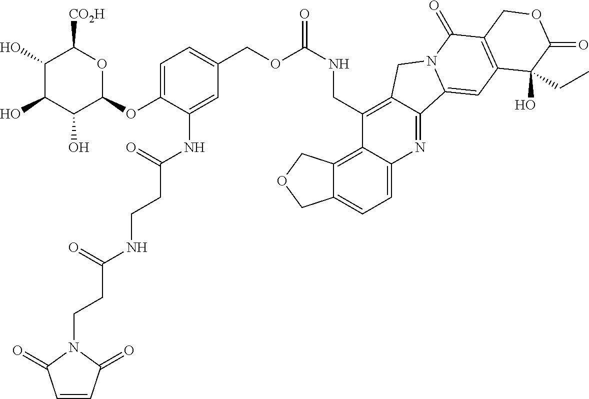 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)COC3] |
| 4m | 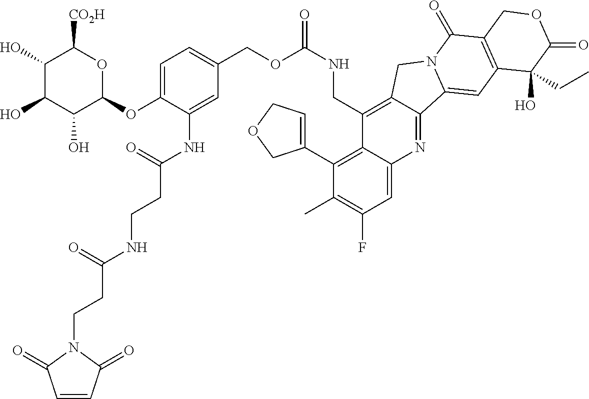 [SMILES: CC[C@@]1(O)C(=O)OCc2c1cc1n(c2=O)Cc2c-1nc1cc(F)c(C)c(C3=CCOC3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4n | 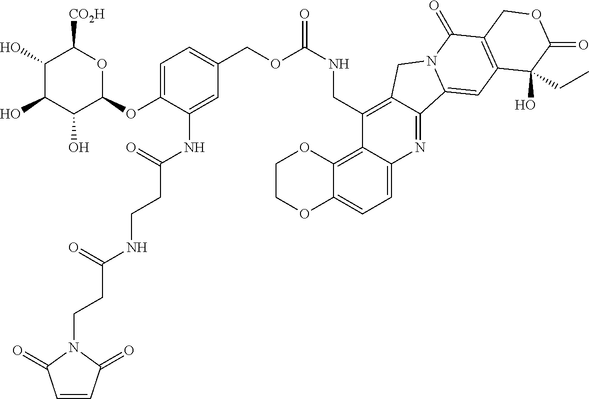 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCCO3] |
| 4o | 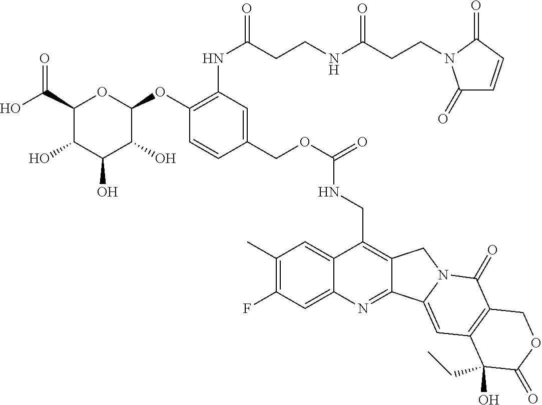 [SMILES: CC[C@@]1(O)C(=O)OCc2c1cc1n(c2=O)Cc2c-1nc1cc(F)c(C)cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4p | 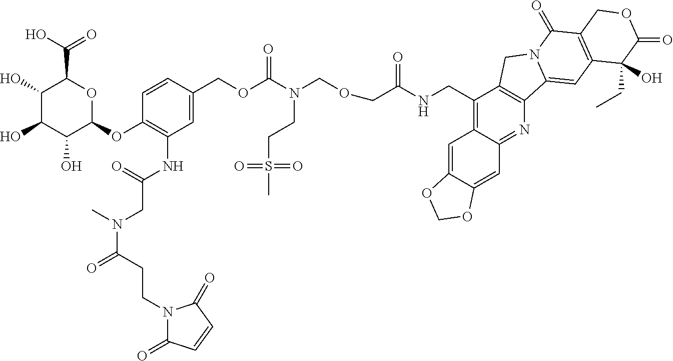 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)OCO3] |
| 4q | 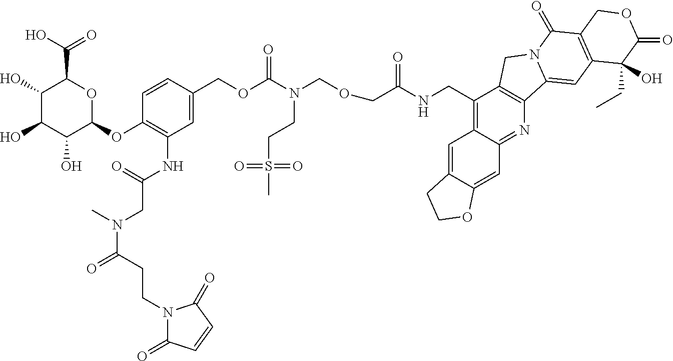 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)CCO3] |
| 4r | 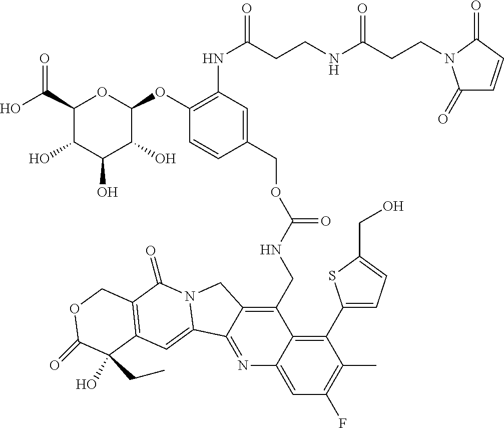 [SMILES: CC[C@@]1(O)C(=O)OCc2c1cc1n(c2=O)Cc2c-1nc1cc(F)c(C)c(-c3ccc(CO)s3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4s | 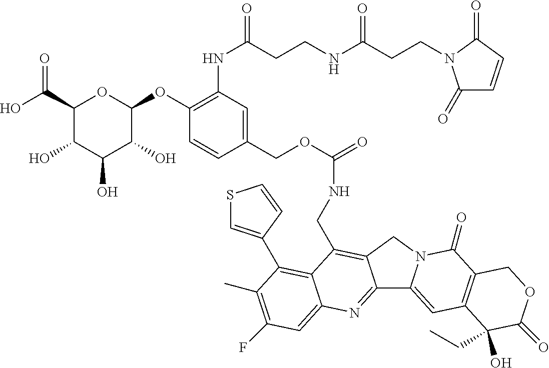 [SMILES: CC[C@@]1(O)C(=O)OCc2c1cc1n(c2=O)Cc2c-1nc1cc(F)c(C)c(-c3ccsc3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4t | 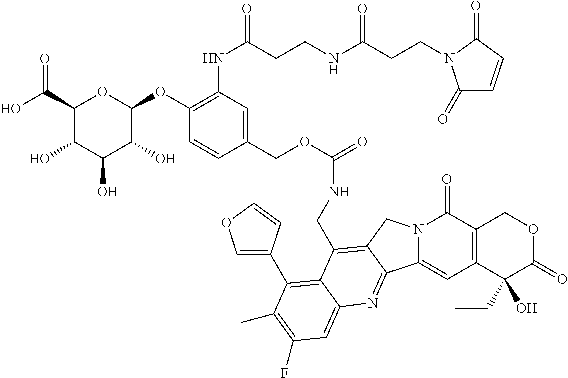 [SMILES: CC[C@@]1(O)C(=O)OCc2c1cc1n(c2=O)Cc2c-1nc1cc(F)c(C)c(-c3ccoc3)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4u | 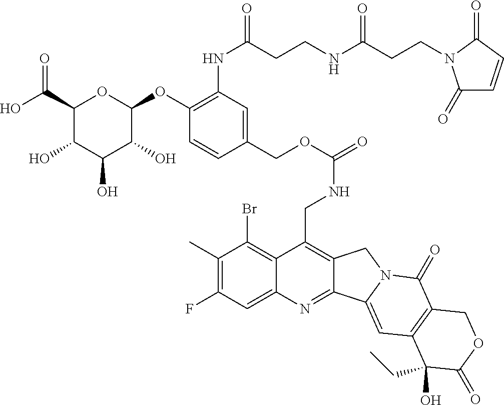 [SMILES: CC[C@@]1(O)C(=O)OCc2c1cc1n(c2=O)Cc2c-1nc1cc(F)c(C)c(Br)c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] |
| 4v | 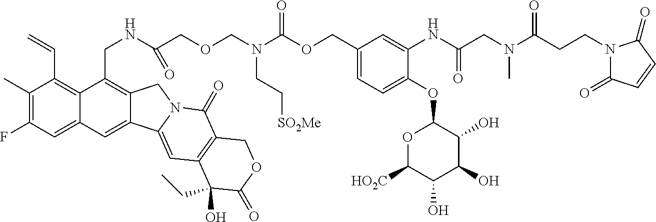 [SMILES: C=Cc1c(C)c(F)cc2cc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4w | 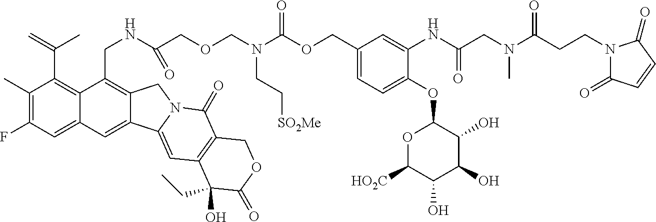 [SMILES: C=C(C)c1c(C)c(F)cc2cc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] |
| 4x | 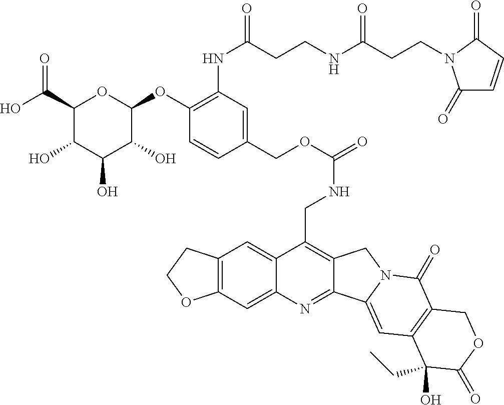 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCO3] |
| 4y | 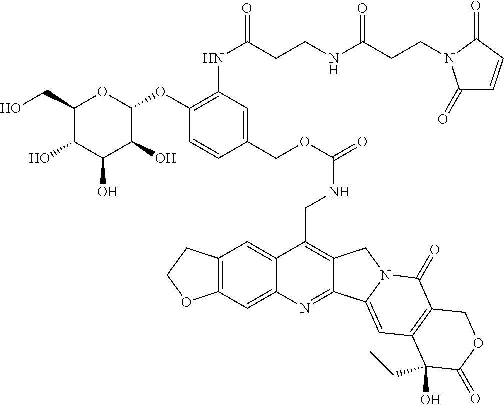 [SMILES: CC[C@@]1(O)C(=O)OCc2c1cc1n(c2=O)Cc2c-1nc1cc3c(cc1c2CNC(=O)OCc1ccc(O[C@H]2O[C@H](CO)[C@@H](O)[C@H](O)[C@@H]2O)c(NC(=O)CCNC(=O)CCN2C(=O)C=CC2=O)c1)CCO3] |
| 4z | 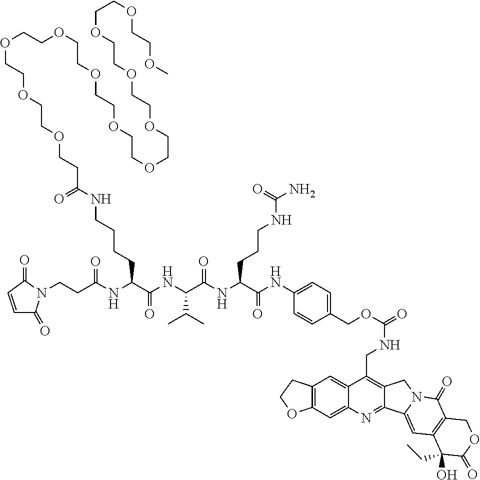 [SMILES: CC[C@@]1(O)C(=O)OCc2c1cc1n(c2=O)Cc2c-1nc1cc3c(cc1c2CNC(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOC)NC(=O)CCN2C(=O)C=CC2=O)C(C)C)cc1)CCO3] |
| 4aa | 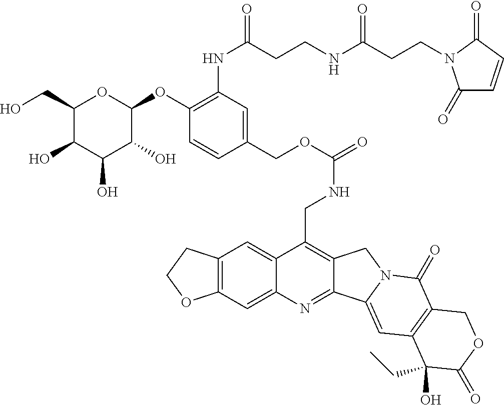 [SMILES: CC[C@@]1(O)C(=O)OCc2c1cc1n(c2=O)Cc2c-1nc1cc3c(cc1c2CNC(=O)OCc1ccc(O[C@@H]2O[C@H](CO)[C@H](O)[C@H](O)[C@H]2O)c(NC(=O)CCNC(=O)CCN2C(=O)C=CC2=O)c1)CCO3] |
| 4ab | 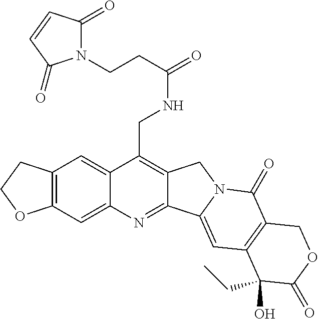 [SMILES: CC[C@@]1(O)C(=O)OCc2c1cc1n(c2=O)Cc2c-1nc1cc3c(cc1c2CNC(=O)CCN1C(=O)C=CC1=O)CCO3] |
| 4ac | 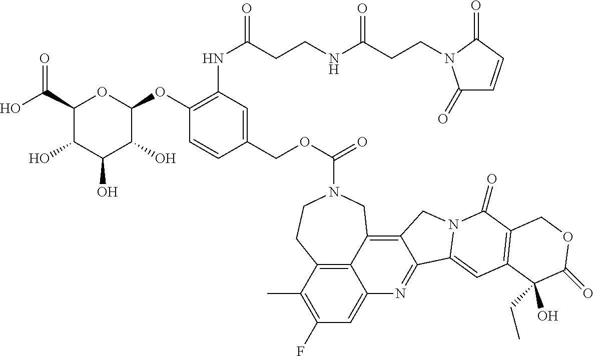 [SMILES: CC[C@@]1(O)C(=O)OCc2c1cc1n(c2=O)Cc2c-1nc1cc(F)c(C)c3c1c2CN(C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CC3] |
| 4ad | 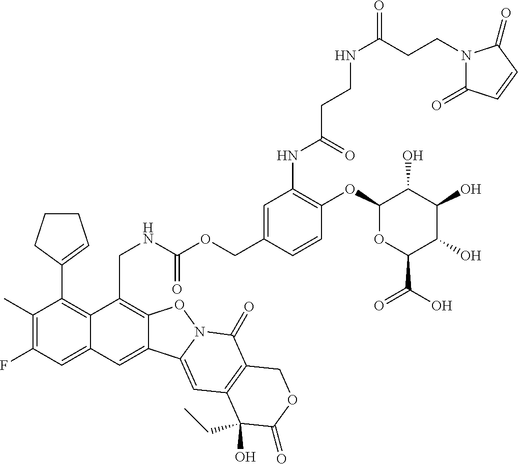 [SMILES: CC[C@@]1(O)C(=O)OCc2c1cc1c3cc4cc(F)c(C)c(C5=CCCC5)c4c(CNC(=O)OCc4ccc(O[C@@H]5O[C@H](C(=O)O)[C@@H](O)[C@H](O)[C@H]5O)c(NC(=O)CCNC(=O)CCN5C(=O)C=CC5=O)c4)c3on1c2=O] |
| 4ae | 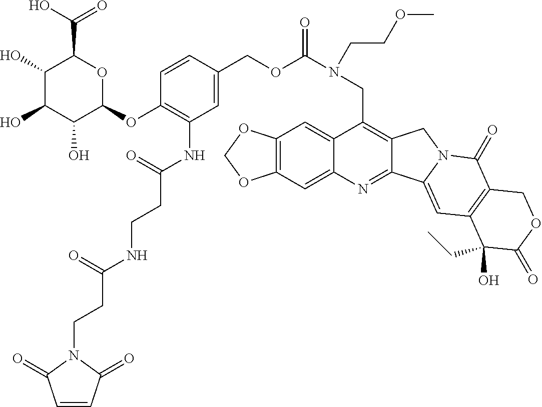 [SMILES: CC[C@@]1(O)C(=O)OCc2c1cc1n(c2=O)Cc2c-1nc1cc3c(cc1c2CN(CCOC)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)OCO3] |

TABLE 5
ADC aggregations levels for camptothecin drug-linkers (DAR =
8) linked to a Ag4 antibody or to an alternative
target-specific antibody referred to as Ag1. ADC aggregation
was determined by Size Exclusion Chromatography (SEC).

| | | | Conc. | |
|---|---|---|---|---|
| ADC | Description | DAR | (mg/mL) | HMW % |
| Ag1-Ex_4 | Glucuronide-Ex_2n | 7.7 | 0.65 | 1.98 |
| Ag4-Ex_4 | Glucuronide-Ex_2n | 8 | 0.86 | 1.65 |
| Ag1-Ex_4a | Glucuronide-Ex_2u | 7.7 | 0.27 | 2.18 |
| Ag4-Ex_4a | Glucuronide-Ex_2u | 7.8 | 0.78 | 1.62 |
| Ag1-Ex_4b | Glucuronide-Ex_2am | 7.8 | 0.46 | 1.92 |
| Ag4-Ex_4b | Glucuronide-Ex_2am | 8 | 0.71 | 1.36 |
| Ag1-Ex_4c | Glucuronide-Ex_2al | 7.6 | 0.55 | 1.98 |
| Ag4-Ex_4c | Glucuronide-Ex_2al | 8 | 0.65 | 1.49 |
| Ag1-Ex_4f | Glucuronide-Ex_1 | 7.5 | 0.87 | 1.82 |
| Ag4-Ex_4f | Glucuronide-Ex_1 | 8 | 0.89 | 1.15 |
| Ag1-Ex_4i | Glucuronide-Ex_2ap | 8 | 0.49 | 1.4 |
| Ag4-Ex_4i | Glucuronide-Ex_2ap | 8 | 0.78 | 0.9 |
| Ag1-Ex_4g | Glucuronide-Ex_2i | 7.9 | 0.65 | 1.4 |
| Ag4-Ex_4g | Glucuronide-Ex_2i | 8 | 0.59 | 0.9 |
| Ag1-Ex_4k | Glucuronide-Ex_2as | 7.8 | 0.76 | 1.5 |
| Ag4-Ex_4k | Glucuronide-Ex_2as | 8 | 0.96 | 1.2 |
| Ag1-Ex_4h | Glucuronide-Ex_2x | 7.5 | 0.62 | 1.5 |
| Ag4-Ex_4h | Glucuronide-Ex_2x | 8 | 0.96 | 1.3 |
| Ag1-Ex_4j | Glucuronide-Ex_2ac | 7.7 | 0.69 | 1.9 |
| Ag4-Ex_4j | Glucuronide-Ex_2ac | 8 | 0.94 | 1.2 |
| Ag4-Ex_4ad | Glucuronide-Ex_6c | 7.9 | 0.78 | 6.4 |
| Ag1-Ex_4ad | Glucuronide-Ex_6c | 8 | 0.84 | 5.4 |
| Ag4-Ex_4ac | Glucuronide-Ex_9 | 7.9 | 0.68 | 2.4 |
| Ag1-Ex_4ac | Glucuronide-Ex_9 | 8 | 0.87 | 1.5 |
| Ag4-Ex_4l | Glucuronide-Ex_2aq | 7.7 | 0.87 | 2.6 |
| Ag1-Ex_4l | Glucuronide-Ex_2aq | 8 | 1.06 | 2.7 |
| Ag4-Ex_4m | Glucuronide-Ex_6d | 8 | 0.69 | 2.5 |
| Ag1-Ex_4m | Glucuronide-Ex_6d | 8 | 0.64 | 1.6 |
| Ag4-Ex_4q | Glucuronide-Ex_15c | 8 | 0.52 | 9.7 |
| Ag1-Ex_4q | Glucuronide-Ex_15c | 8 | 0.65 | 15.62 |

### Example 5: Preparation of Compounds 5a-5c and 2Ak

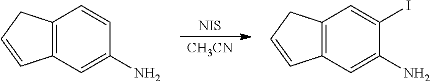

To a solution of 1H-indene-5-amine (3 g, 22.92 mmol) in acetonitrile (MeCN, 135 mL) was added a solution of NIS (5.21 g, 23.15 mmol) in acetonitrile (MeCN, 30 mL) dropwise at −15° C. The mixture was stirred at −15° C. for 15 min. The reaction mixture was quenched by addition of a saturated Na₂S₂O₃ (150 mL) at −15° C., and then extracted with ethyl acetate (3×500 mL). The combined organic layer was dried over Na₂SO₄, filtered, and concentrated under reduced pressure to give a residue. The residue was purified by flash silica gel chromatography Eluent of 0-10% Ethyl acetate/Petroleum ether to afford product 6-iodo-1H-inden-5-amine (10.1 g, yield: 28.6%)

¹H NMR (400 MHz, CDCl₃): 5=7.72 (s, 1H), 6.85 (s, 1H), 6.77-6.69 (m, 1H), 6.52 (td, J=1.6, 5.6 Hz, 1H), 4.05 (br s, 2H), 3.30 (s, 2H).

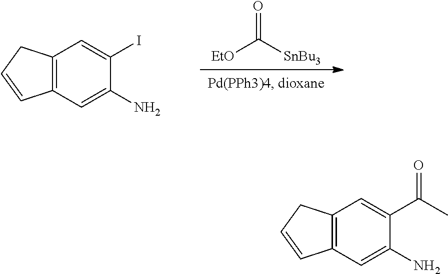

To a solution of 6-iodo-1H-inden-5-amine (4.8 g, 18.67 mmol) in dioxane (120 mL) was added tributyl(1-ethoxyvinyl)stannane (11.47 g, 31.74 mmol). Then Pd(PPh₃)₄ (2.16 g, 1.87 mmol) was added under nitrogen. The mixture was stirred at 100° C. for 16 h. The mixture was cooled to room temperature, then HCl (2M, 300 mL) was added and stirred for 10 min. The reaction mixture was extracted with ethyl acetate (2×300 mL). And a solution of Na₂CO₃ in H2O was added to aqueous phase to pH>7. Then the mixture was extracted with ethyl acetate (3×500 mL). The combined organic phase was dried over Na₂SO₄ and concentrated. The residue was purified by flash silica gel chromatography, eluent of 0-6% ethyl acetate/Petroleum ether gradient gave a product 1-(5-amino-1H-inden-6-yl)-2-chloroethan-1-one (320 mg, 5%).

¹H NMR (400 MHz, CDCl₃): 5=7.76 (s, 1H), 6.76 (s, 2H), 6.68 (s, 1H), 3.39 (d, J=0.4 Hz, 2H), 2.60 (s, 3H).

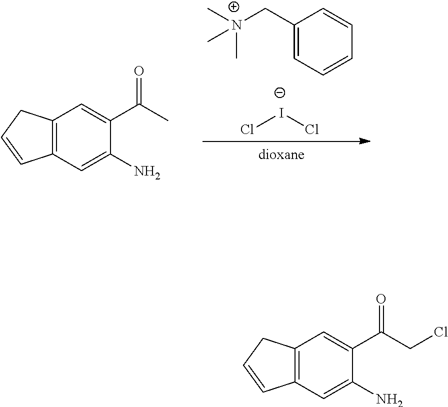

To a solution of 1-(5-amino-1 H-inden-6-yl)ethan-1-one (80 mg, 461.87 umol) in dioxane (10 mL) was added benzyltrimethylammonium dichloroiodate (385.81 mg, 1.11 mmol). The mixture was stirred at 70° C. for 6 h. The mixture was poured into a solution of Na₂S₂O₃ and NaHCO₃, stirred at 25° C. for 5 min. The phases were separated, and the aqueous phase was extracted twice with ethyl acetate (80 mL). The combined extracts were dried over Na₂SO₄ and concentrated to give a residue. The residue was purified by preparative HPLC to afford product 1-(5-amino-1 H-inden-6-yl)-2-chloroethan-1-one (34 mg, 9%).

General Method UPLC-MS: t_{R}=1.80 min. m/z (ES+) 208.05 (M+H)⁺, found 208.01

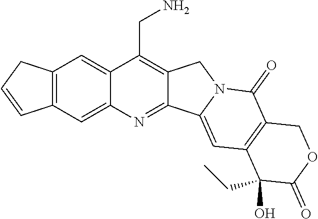 [SMILES: CC[C@@]1(O)C(=O)OCc2c1cc1n(c2=O)Cc2c-1nc1cc3c(cc1c2CN)CC=C3]

(5-amino-1H-inden-6-yl)-2-chloroethan-1-one was used to prepare compound 5 using similar procedures as defined in Example 2.

TABLE 6
Compounds 5a-5c and 2ak were made following the procedures outlined
for Compound 5.

| | | | Calc. | | Rt |
|---|---|---|---|---|---|
| No. | Structure | Exact Mass | [M + H]+ | m/z | (min) |
| 5 | 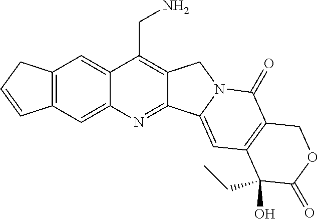 [SMILES: CC[C@@]1(O)C(=O)OCc2c1cc1n(c2=O)Cc2c-1nc1cc3c(cc1c2CN)CC=C3] | 415.15 | 416.16 | 416.75 | 2.98 |
| 5a | 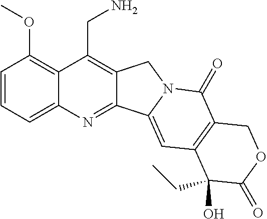 [SMILES: CC[C@@]1(O)C(=O)OCc2c1cc1n(c2=O)Cc2c-1nc1cccc(OC)c1c2CN] | 407.15 | 408.16 | 407.85 | 1.00 |
| 5b | 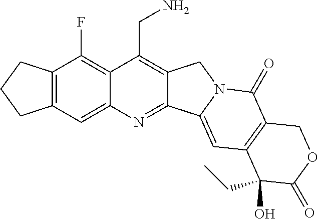 [SMILES: CC[C@@]1(O)C(=O)OCc2c1cc1n(c2=O)Cc2c-1nc1cc3c(c(F)c1c2CN)CCC3] | 435.16 | 436.17 | 436.03 | 1.10 |
| 5c | 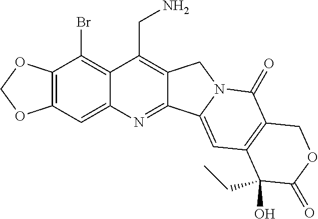 [SMILES: CC[C@@]1(O)C(=O)OCc2c1cc1n(c2=O)Cc2c-1nc1cc3c(c(Br)c1c2CN)OCO3] | 499.04 | 500.05 | 500.33 | 1.04 |
| 2ak | 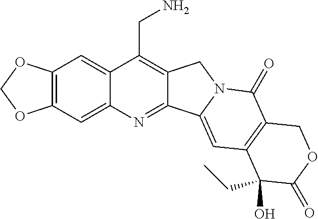 [SMILES: CC[C@@]1(O)C(=O)OCc2c1cc1n(c2=O)Cc2c-1nc1cc3c(cc1c2CN)OCO3] | 419.15 | 420.16 | 420.6 | 0.79 |

### Example 6: Preparation of Compounds 6a-6i, 2Ai, 2Aj, 2a1 and 2am

tert-butyl (S)-((10-bromo-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate

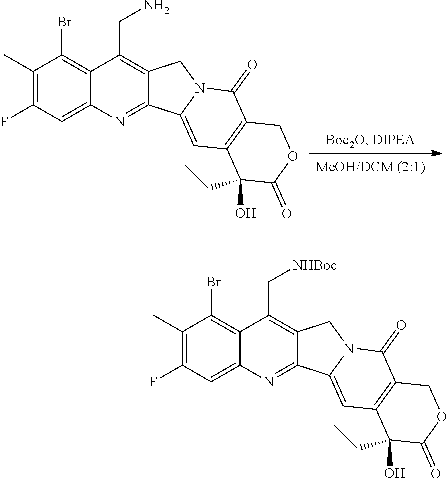

In a 4 mL vial equipped with a stir bar, (S)-11-(aminomethyl)-10-bromo-4-ethyl-8-fluoro-4-hydroxy-9-methyl-1,12-dihydro-14H-pyrano [3%4%6,7]indolizino [1,2-b]quinoline-3,14(4H)-dione (Compound 2q, 230 mg, 0.47 mmol) was added into McOH/DCM (2:1, 1.5 mL). Boc20 (113 mg, 0.52 mmol) and DIPEA (164 uL, 0.94 mmol) were added to the above solution at RT. The resulting solution was stirred at RT for 2 h. The reaction solution was concentrated in vacuo and residue was purified by silica gel column chromatography (0-20% MeOH:DCM) to afford product (193 mg, 70% yield).

General Method UPLC-MS: t_{R}=2.26 min, m/z (ES+) 589.43 (M+H)⁺, found 589.75. tert-butyl (S)-((4-ethyl-8-fluoro-4hydroxy-9-methyl-10-(2-methylprop-1-en-1-yl)-3,14-dioxo-3,4,12,14-tetrahydro-1H-pyrano[3′,4′:6,7]indolizino[1,2-b]quinolin-11-yl)methyl)carbamate

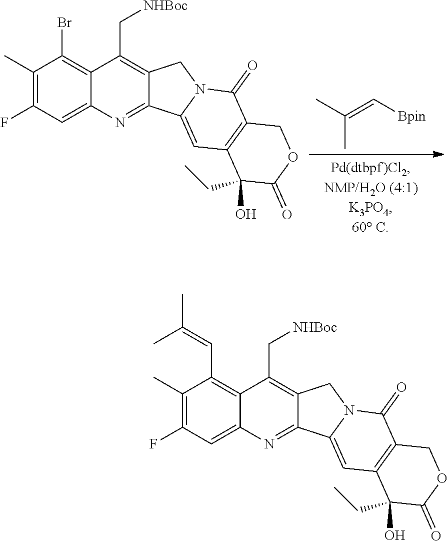

To a mixture of tert-butyl (S)-((10-bromo-4-ethyl-8-fluoro-4-hydroxy-9-methyl-3,14-dioxo-3,4,12,14-tetrahydro-1 H-pyrano[3′,4′:6,7]indolizino[1,2-b]quinolin-11-yl)methyl)cait amate (15 mg, 0.03 mmol) and 0.5 M K₃PO₄ (204 uL, 0.10 mmol) in NMP (0.4 mL) was added Pd(dtbpf)Cl₂ (1.8 mg, 2.50 umol) under nitrogen. The mixture was degassed and purged with N₂ for 3 times, and 4,4,5,5-tetramethyl-2-(2-methylprop-1-enyl)-1,3,2-dioxaborolane (18.6 mg, 0.10 mmol) was added via syringe, followed by addition of degassed H₂O (0.1 mL). Then the mixture was stirred at 60° C. for 2 h under nitrogen. The reaction solution was added AcOH to pH=5, filtered and the filtrate was purified by preparative HPLC to afford product (7.6 mg, yield: 53%). General Method UPLC-MS: t_{R}=2.33 min, m/z (ES+) 563.63 (M+H)⁺, found 563.84.

(S)-11-(aminomethyl)-4-ethyl-8-fluoro-4-hydroxy-9-methyl-10-(2-methylprop-1-en-1-yl)-1,12-dihydro-14H-pyrano[3′,4′:6,7]indolizino[1,2-b]quinoline-3,14(4H)-dione

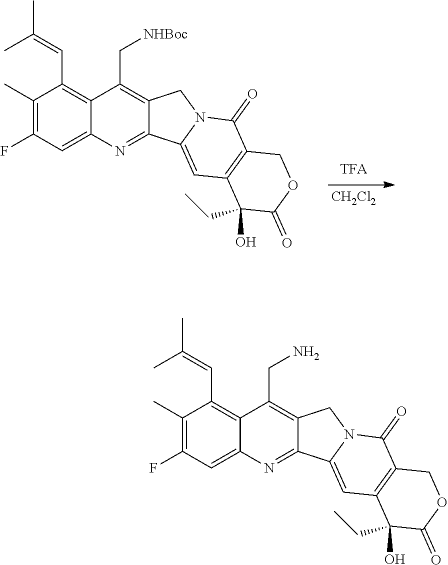

tert-butyl (S)-((4-ethyl-8-fluoro-4-hydroxy-9-methyl-10-(2-methylprop-1-en-1-yl)-3,14-dioxo-3,4,12,14-tetrahydro-1 H-pyrano[3′,4′:6,7]indolizino[1,2-b]quinolin-11-yl)methyl)cait amate (7.6 mg, 13.5 umol) was dissolved in 20% TFA/DCM (0.5 mL) at RT. The reaction solution was stirred at RT for 1 h. The solvent was removed in vacuo and the material purified by preparative HPLC to afford product Compound 6 (3.4 mg, yield: 55%). General Method UPLC-MS: t_{R}=1.44 min, m/z (ES+) 463.19 (M+H)⁺, found 463.85.

TABLE 7
Compounds 6a-6i, 2ai, 2aj, 2al and 2am were made following the
procedures outlined for Compound 6.

| | | | Calc. | | Rt |
|---|---|---|---|---|---|
| No. | Structure | Exact Mass | [M + H]+ | m/z | (min) |
| 6 | 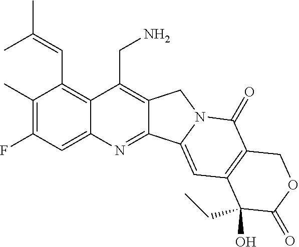 [SMILES: CC[C@@]1(O)C(=O)OCc2c1cc1n(c2=O)Cc2c-1nc1cc(F)c(C)c(C=C(C)C)c1c2CN] | 463.19 | 464.20 | 463.85 | 1.44 |
| 6a | 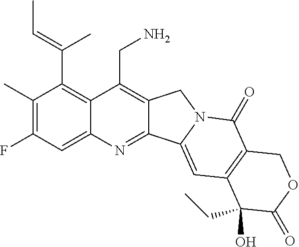 [SMILES: C/C=C(\C)c1c(C)c(F)cc2nc3c(c(CN)c12)Cn1c-3cc2c(c1=O)COC(=O)[C@]2(O)CC] | 463.19 | 464.20 | 464.92 | 1.35 |
| 6b | 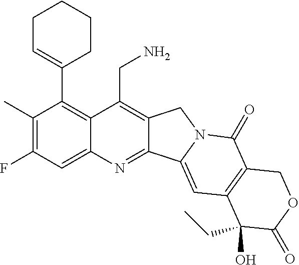 [SMILES: CC[C@@]1(O)C(=O)OCc2c1cc1n(c2=O)Cc2c-1nc1cc(F)c(C)c(C3=CCCCC3)c1c2CN] | 489.21 | 490.22 | 489.77 | 1.47 |
| 6c | 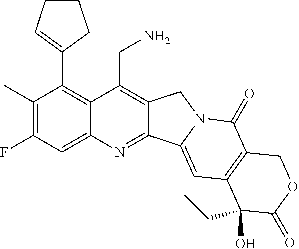 [SMILES: CC[C@@]1(O)C(=O)OCc2c1cc1n(c2=O)Cc2c-1nc1cc(F)c(C)c(C3=CCCC3)c1c2CN] | 475.19 | 476.20 | 475.68 | 1.35 |
| 6d | 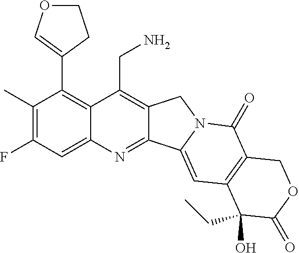 [SMILES: CC[C@@]1(O)C(=O)OCc2c1cc1n(c2=O)Cc2c-1nc1cc(F)c(C)c(C3=COCC3)c1c2CN] | 477.17 | 478.18 | 477.67 | 1.16 |
| 6e | 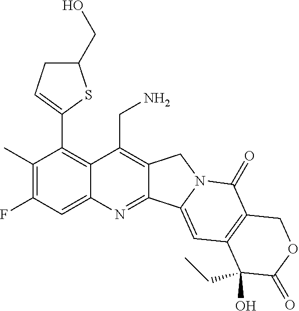 [SMILES: CC[C@@]1(O)C(=O)OCc2c1cc1n(c2=O)Cc2c-1nc1cc(F)c(C)c(C3=CCC(CO)S3)c1c2CN] | 521.14 | 522.15 | 521.72 | 1.24 |
| 6f | 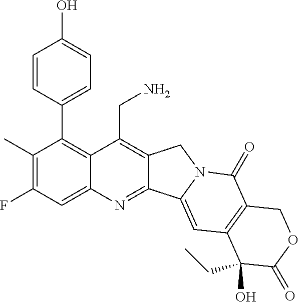 [SMILES: CC[C@@]1(O)C(=O)OCc2c1cc1n(c2=O)Cc2c-1nc1cc(F)c(C)c(-c3ccc(O)cc3)c1c2CN] | 501.17 | 502.18 | 501.59 | 1.26 |
| 6g | 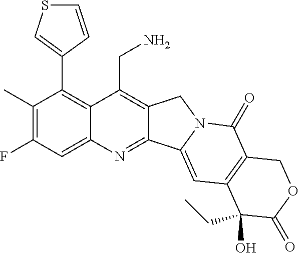 [SMILES: CC[C@@]1(O)C(=O)OCc2c1cc1n(c2=O)Cc2c-1nc1cc(F)c(C)c(-c3ccsc3)c1c2CN] | 491.13 | 492.14 | 491.63 | 1.31 |
| 6h | 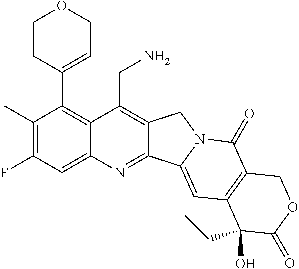 [SMILES: CC[C@@]1(O)C(=O)OCc2c1cc1n(c2=O)Cc2c-1nc1cc(F)c(C)c(C3=CCOCC3)c1c2CN] | 491.18 | 492.20 | 491.63 | 1.18 |
| 6i | 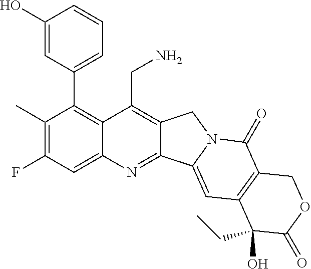 [SMILES: CC[C@@]1(O)C(=O)OCc2c1cc1n(c2=O)Cc2c-1nc1cc(F)c(C)c(-c3cccc(O)c3)c1c2CN] | 501.17 | 502.18 | 501.66 | 1.28 |
| 6j | 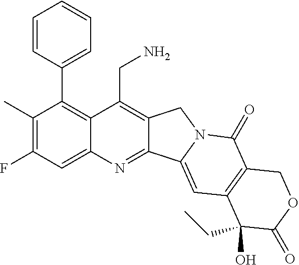 [SMILES: CC[C@@]1(O)C(=O)OCc2c1cc1n(c2=O)Cc2c-1nc1cc(F)c(C)c(-c3ccccc3)c1c2CN] | 485.18 | 486.18 | 485.71 | 1.29 |
| 6k | 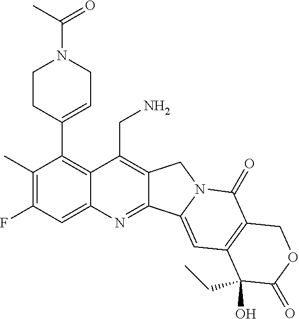 [SMILES: CC[C@@]1(O)C(=O)OCc2c1cc1n(c2=O)Cc2c-1nc1cc(F)c(C)c(C3=CCN(C(C)=O)CC3)c1c2CN] | 532.21 | 533.22 | 532.75 | 1.09 |
| 6l | 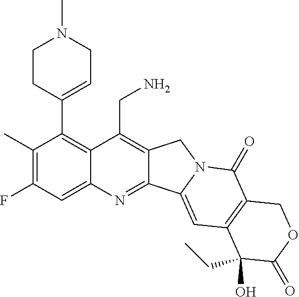 [SMILES: CC[C@@]1(O)C(=O)OCc2c1cc1n(c2=O)Cc2c-1nc1cc(F)c(C)c(C3=CCN(C)CC3)c1c2CN] | 504.22 | 505.23 | 504.91 | 0.84 |
| 6m | 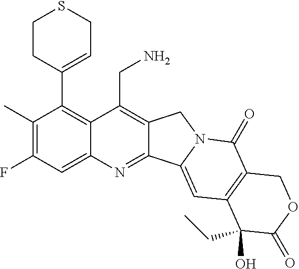 [SMILES: CC[C@@]1(O)C(=O)OCc2c1cc1n(c2=O)Cc2c-1nc1cc(F)c(C)c(C3=CCSCC3)c1c2CN] | 507.16 | 508.17 | 508.08 | 1.28 |
| 6n | 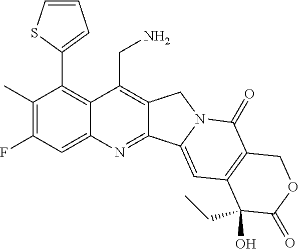 [SMILES: CC[C@@]1(O)C(=O)OCc2c1cc1n(c2=O)Cc2c-1nc1cc(F)c(C)c(-c3cccs3)c1c2CN] | 491.13 | 492.14 | 491.75 | 1.34 |
| 6o | 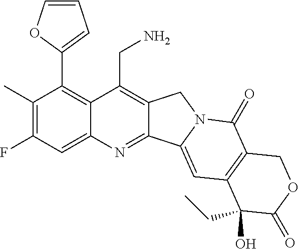 [SMILES: CC[C@@]1(O)C(=O)OCc2c1cc1n(c2=O)Cc2c-1nc1cc(F)c(C)c(-c3ccco3)c1c2CN] | 475.15 | 476.15 | 475.43 | 1.23 |
| 6p | 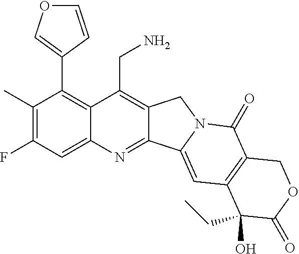 [SMILES: CC[C@@]1(O)C(=O)OCc2c1cc1n(c2=O)Cc2c-1nc1cc(F)c(C)c(-c3ccoc3)c1c2CN] | 475.15 | 476.16 | 475.81 | 1.25 |
| 2ai | 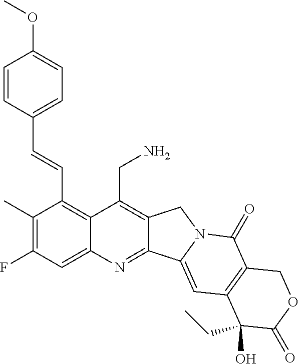 [SMILES: CC[C@@]1(O)C(=O)OCc2c1cc1n(c2=O)Cc2c-1nc1cc(F)c(C)c(/C=C/c3ccc(OC)cc3)c1c2CN] | 541.20 | 542.21 | 542.05 | 1.32 |
| 2aj | 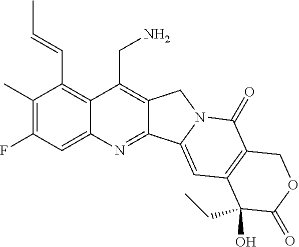 [SMILES: C/C=C/c1c(C)c(F)cc2nc3c(c(CN)c12)Cn1c-3cc2c(c1=O)COC(=O)[C@]2(O)CC] | 449.18 | 450.19 | 450.19 | 1.11 |
| 2al | 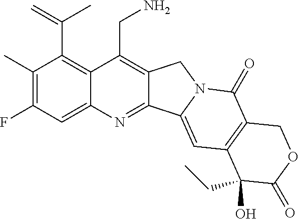 [SMILES: C=C(C)c1c(C)c(F)cc2nc3c(c(CN)c12)Cn1c-3cc2c(c1=O)COC(=O)[C@]2(O)CC] | 449.18 | 450.19 | 450.38 | 1.06 |
| 2am | 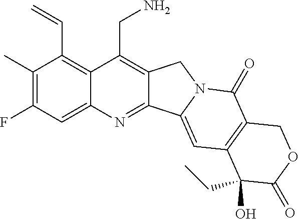 [SMILES: C=Cc1c(C)c(F)cc2nc3c(c(CN)c12)Cn1c-3cc2c(c1=O)COC(=O)[C@]2(O)CC] | 435.16 | 436.17 | 436.22 | 1.03 |

### Example 7: Preparation of Compounds 33 and 33a

(19S)-10-(aminomethyl)-19-ethyl-19-hydroxy-7-vinyl-17-oxa-3,13-diazapentacyclo[11.8.0.02,11.04,9.015,20]henicosa-1(21),2,4,6,8,10,15(20)-heptaene-14,18-dione

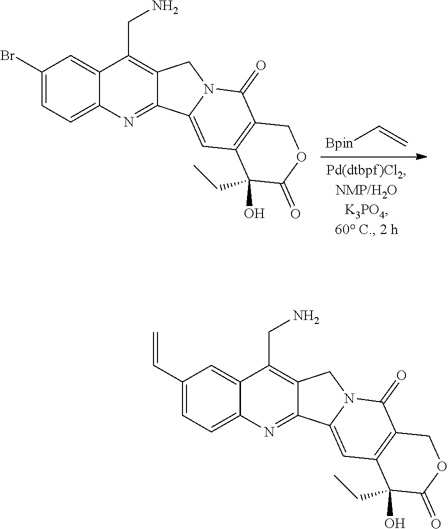

To a mixture of (19S)-10-(aminomethyl)-7-bromo-19-ethyl-19-hydroxy-17-oxa-3,13-diazapentacyclo[11.8.0.02,11.04,9.015,20]henicosa-1(21),2,4,6,8,10,15(20)-heptaene-14,18-dione (15 mg, 0.03 mmol) and 0.5 M K₃PO₄ (263 uL, 0.13 mmol) in NMP (0.4 mL) was added Pd(dtbpf)Cl₂ (2.3 mg, 3.3 umol) under nitrogen. The mixture was degassed and purged with N₂ for 3 times, and 4,4,5,5-tetramethyl-2-vinyl-1,3,2-dioxaborolane (22.3 uL, 0.13 mmol) was added via syringe, followed by addition of degassed H₂O (0.1 mL). Then the mixture was stirred at 60° C. for 2 h under nitrogen. The reaction solution was added AcOH to pH=5, filtered and the filtrate was purified by preparative HPLC to afford product

Compound 33 (3.6 mg, yield: 27%). General Method UPLC-MS: t_{R}=1.08 min, m/z (ES+) 403.44 (M+H)⁺, found 403.73.

TABLE 8
Compound 33a was made following the procedure outlined for
Compound 33.

| | | | Calc. | | Rt |
|---|---|---|---|---|---|
| No. | Structure | Exact Mass | [M + H]+ | m/z | (min) |
| 33 | 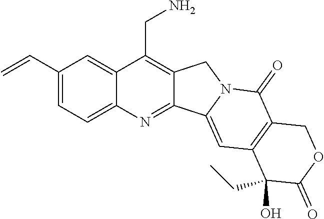 [SMILES: C=Cc1ccc2nc3c(c(CN)c2c1)Cn1c-3cc2c(c1=O)COC(=O)[C@]2(O)CC] | 403.15 | 404.16 | 403.73 | 1.08 |
| 33a | 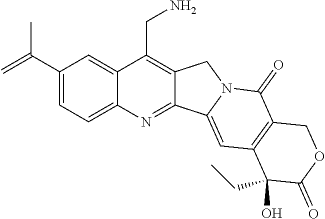 [SMILES: C=C(C)c1ccc2nc3c(c(CN)c2c1)Cn1c-3cc2c(c1=O)COC(=O)[C@]2(O)CC] | 417.17 | 418.18 | 418.15 | 1.19 |

### Example 8: Preparation of Compound 34

(S)-11-(aminomethyl)-4-ethyl-10-ethynyl-8-fluoro-4-hydroxy-9-methyl-1,12-dihydro-14H-pyrano[3′,4′:6,7]indolizino[1,2-b]quinoline-3,14(4H)-dione

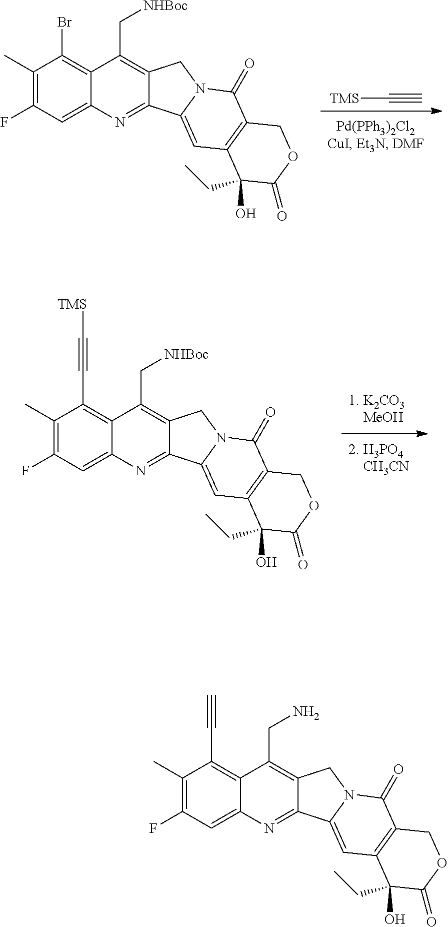

Tert-butyl (S)-((10-bromo-4-ethyl-8-fluoro-4-hydroxy-9-methyl-3,14-dioxo-3,4,12,14-tetrahydro-1 H-pyrano[3′,4′:6,7]indolizino[1,2-b]quinolin-11-yl)methyl)carbamate (10 mg, 20 umol), trimethylsilylacetylene (4 mg, 41 umol) and DMF (0.2 mL) were added to a 4 mL vial equipped with stir bar. CuI (1.9 mg, 10 umol), Pd(PPh₃)₂C₁₂ (0.8 mg, 4 umol) and triethylamine (0.2 mL) were added to the mixture. The reaction system was degassed and recharged with nitrogen. The mixture was stirred at 25° C. for 6 h under nitrogen. LCMS showed the reaction was completed. The reaction mixture was filtered and concentrated in vacuo. The crude product was purified by preparatory HPLC.

Tert-butyl (S)-((4-ethyl-8-fluoro-4-hydroxy-9-methyl-3,14-dioxo-10-((trimethylsilyl)ethynyl)-3,4,12,14-tetrahydro-1H-pyrano[3′,4′:6,7]indolizino[1,2-b]quinolin-11-yl)methyl)carbamate (3.1 mg, 6 umol) and McOH (0.2 mL) was added to a 4 mL vial equipped with stir bar. K₂CO₃ (2.4 mg, 18 umol) was added to the solution at 0° C. and the reaction was warmed to RT and stirred for 3 h. Reaction was then concentrated to dryness in vacuo, and the crude product was dissolved in a mixture of H₃PO₄ (0.17 mL), acetonitrile (0.17 mL) and H₂O (0.17 mL). The reaction solution was filtered and prepped by HPLC to afford product Compound 34 (0.3 mg, 4.5%).

General Method UPLC-MS: t_{R}=1.23 min. m/z (ES+) 434.15 (M+H)⁺, found 434.49

### Example 9: Preparation of Compound 9

(S)-10-ethyl-6-fluoro-10-hydroxy-5-methyl-2,3,4,10,13,16-hexahydro-14H-azepino[3,4,5-de]pyrano[3′,4′:6,7]indolizino[1,2-b]quinoline-11,14(1H)-dione

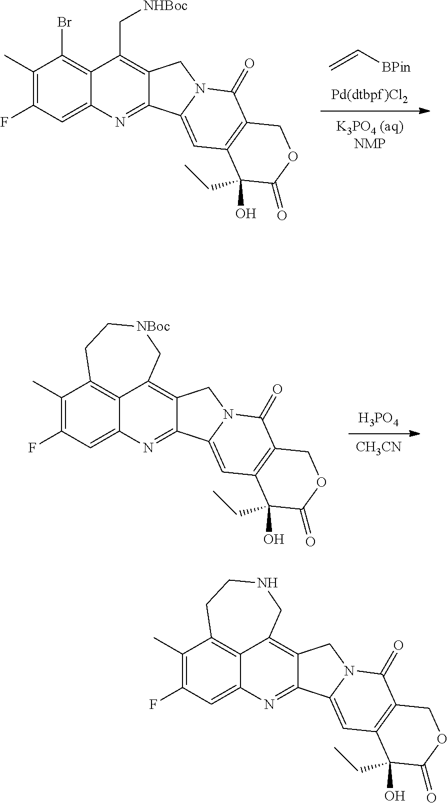

To a mixture of tert-butyl (S)-((10-bromo-4-ethyl-8-fluoro-4-hydroxy-9-methyl-3,14-dioxo-3,4,12,14-tetrahydro-1 H-pyrano[3′,4′:6,7]indolizino[1,2-b]quinolin-11-yl)methyl)carbamate (100 mg, 17 umol) and K₃PO₄ (180 mg, 0.85 mmol) in NMP (3 mL) was added Pd(dtbpf)C₁₂ (11 mg, 17 umol) under nitrogen. 4,4,5,5-tetramethyl-2-vinyl-1,3,2-dioxaborolane (104 mg, 0.7 mmol) was added via syringe, followed by addition of degassed H₂O (0.6 mL). Then the mixture was stirred at 80° C. for 16 h under nitrogen. The reaction solution was acidified with AcOH to pH 5, and then poured into water and extracted with ethyl acetate for 3 times. The combined organic phase was washed with brine, dried over Na₂SO₄, filtered and concentrated to give a residue. The residue was purified by silica gel column chromatography (petroleum ether: ethyl acetate=10: 1 to 0:1).

H₃PO₄ (0.17 mL), acetonitrile (0.17 mL) and H₂O (0.17 mL) were premixed, and tert-butyl (5)-10-ethyl-6-fluoro-10-hydroxy-5-methyl-11,14-dioxo-3,4,10,11,14,16-hexahydro-13H-azepino[3,4,5-de]pyrano[3′,4′:6,7]indolizino[1,2-b]quinoline-2(1 H)-carboxylate (55 mg, 0.10 mmol) was added at 25° C. The resulting solution was stirred at 25° C. for 16 h. LCMS showed the reaction was completed. The reaction solution was filtered and purified by prep HPLC to afford product Compound 9 (20 mg, 45%).

General Method UPLC-MS: t_{R}=1.06 min. m/z (ES+) 436.17 (M+H)⁺, found 436.14.

### Example 10: Preparation of Compound 10

(S)-11-(aminomethyl)-4,10-diethyl-8-fluoro-4-hydroxy-9-methyl-1,12-dihydro-14H-pyrano[3′,4′:6,7]indolizino[1,2-b]quinoline-3,14(4H)-dione

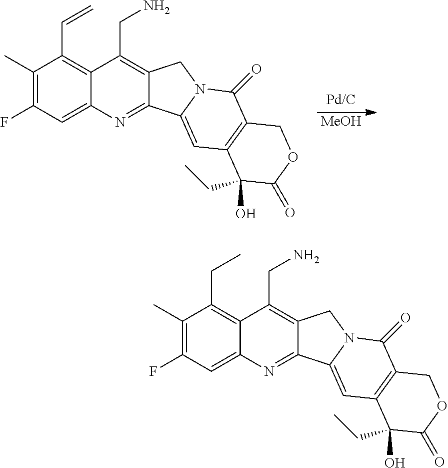

In a 4 mL vial equipped with a stir bar, the (S)-11-(aminomethyl)-4-ethyl-8-fluoro-4-hydroxy-9-methyl-10-vinyl-1,12-dihydro-14H-pyrano[3′,4′:6,7]indolizino[1,2-b]quinoline-3,14(4H)-dione was dissolved (Compound 2am, 5.4 mg, 0.012 mmol) in methanol (300 uL) and palladium on carbon was added (0.26 mg, 0.003 mmol). Reaction was stirred for 2 h at RT. Reaction was filtered, and solvent removed in vacuo. Residue was purified by preparative HPLC to afford product Compound 10 (1.9 mg, yield=35.2%). General Method UPLC-MS: t_{R}=1.20 min. m/z (ES+) 438.18 (M+H)⁺, found 438.41.

### Example 11. Preparation of Compounds 11 and 11 a

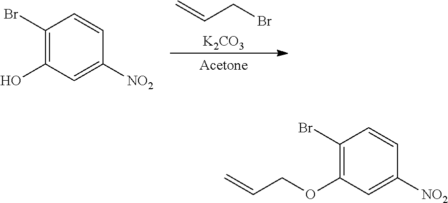

2-bromo-5-nitro-phenol (1.00 eq, 500 mg, 2.29 mmol) was dissolved in Acetone (20 mL). Ally bromide (1.50 eq, 0.30 mL, 3.44 mmol) was added followed by dipotassium;carbonate (2.00 eq, 634 mg, 4.59 mmol). The reaction was heated at 60° C. for 2h at which point complete conversion was observed. The reaction was cooled to room temperature and concentrated in vacuo. The residue was dissolved in EtOAc (50 mL), washed with water (3×50 mL), washed brine, dried MgSO4, filtered and concentrated in vacuo to afford the desired product as a colorless solid 2-allyloxy-1-bromo-4-nitro-benzene (544 mg, 2.11 mmol, 91.94% yield). Used in the next step without further purification.

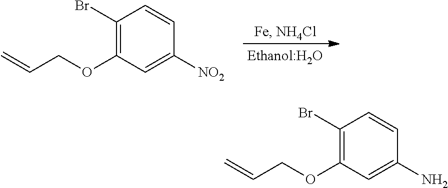

2-allyloxy-1-bromo-4-nitro-benzene (1.00 eq, 468 mg, 1.81 mmol), ammonium chloride (10.0 eq, 969 mg, 18.1 mmol), and iron (10.0 eq, 1012 mg, 18.1 mmol) were dissolved in Ethanol (29.881 mL) and Water (7.4702 mL). The reaction was stirred at 80° C. for 2h at which point complete conversion was observed. The reaction was filtered, and the eluent was concentrated in vacuo. The residue was diluted with EtOAc, washed with water (2×50 mL), washed brine (30 mL), dried MgSO4, filtered and concentrated in vacuo to afford the desired product as a yellow oil g 3-allyloxy-4-bromo-aniline (401 mg, 1.76 mmol, 96.95% yield). Rt=1.51 min General Method UPLC. MS (m/z) [M+H]⁺ calc. for C₉H₉BrNO 228.00, found 228.07.

¹H NMR (500 MHz, Chloroform-d) δ 7.28 (d, J=8.4 Hz, 1H), 6.28 (d, J=2.5 Hz, 1H), 6.22 (dd, J=8.4, 2.5 Hz, 1H), 6.08 (ddt, J=17.2, 10.5, 4.9 Hz, 1H), 5.50 (dq, J=17.3, 1.7 Hz, 1H), 5.32 (dq, J=10.6, 1.5 Hz, 1H), 4.57 (dt, J=4.9, 1.7 Hz, 2H), 3.77 (s, 2H).

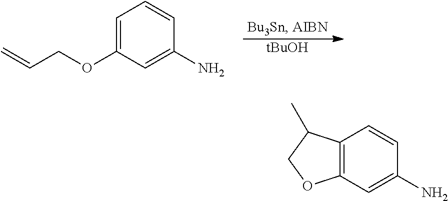

3-allyloxy-4-bromo-aniline (1.00 eq, 401 mg, 1.76 mmol) was dissolved in tert-butanol (17.563 mL). tributylstannane (10.0 eq, 4.7 mL, 17.6 mmol) was added to the reaction followed by 2,2′-Azobis(2-methylpropionitrile) (0.0600 eq, 17 mg, 0.105 mmol). The reaction was stirred at 80° C. for 90 minutes at which point conversion to the desired product was observed. The reaction was cooled to room temperature and 10% KF (15 mL) was added the reaction was stirred for 30 minutes. EtOAC (100 mL) was added, washed sat NaHCO₃(2×100 mL), washed brine (50 mL), dried MgSO4 filtered and concentrated in vacuo. The residue was purified by FCC 0-50% EtOAc in Hex. Fractions containing the desired product were concentrated in vacuo to afford a colorless oil 3-methyl-2,3-dihydrobenzofuran-6-amine (227 mg, 1.52 mmol, 86.52% yield). Rt=0.59 min General Method UPLC. MS (m/z) [M+H]⁺ calc. for C₉H₁₂NO 150.09, found 149.82 ¹H NMR (500 MHz, Chloroform-d) δ 6.91 (d, J=7.8 Hz, 1H), 6.25-6.20 (m, 1H), 6.18 (d, J=2.1 Hz, 1H), 4.65 (t, J=8.7 Hz, 1H), 4.03 (t, J=7.9 Hz, 1H), 3.76 (m, 2H), 3.44 (h, J=7.1 Hz, 1H), 1.27 (d, J=6.8 Hz, 3H).

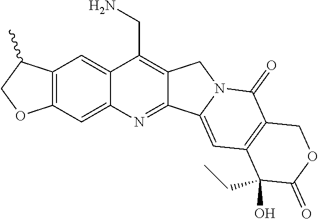 [SMILES: CC[C@@]1(O)C(=O)OCc2c1cc1n(c2=O)Cc2c-1nc1cc3c(cc1c2CN)C(C)CO3]

(5-amino-1H-inden-6-yl)-2-chloroethan-1-one was used to prepare Compound 11 and Compound 11a using similar procedures as defined in Example 2. Two separable diastereomer products were isolated:

Compound 11(5S)-14-(aminomethyl)-5-ethyl-5-hydroxy-18-methyl-7,20-dioxa-11,24 diazahexacyclo[11.11.0.02,11.04,9.015,23.017,21]tetracosa-1(24),2,4(9),13,15(23),16,21-heptaene-6,10-dione (16 mg, 0.0369 mmol, 16.12% yield). Rt=1.04 min General Method UPLC. MS (m/z) [M+H]⁺ calc. for C₂₄H₂₃N₃O₅ 434.17, found 434.25.

Compound 11a (5S)-14-(aminomethyl)-5-ethyl-5-hydroxy-18-methyl-7,20-dioxa-11,24-diazahexacyclo[11.11.0.02,11.04,9.015,23.017,21]tetracosa-1(24),2,4(9),13,15(23),16,21-heptaene-6,10-dione (19 mg, 0.0430 mmol, 18.75% yield). Rt=1.03 min General Method UPLC. MS (m/z) [M+H]⁺ calc. for C₂₄H₂₃N₃O₅ 434.17, found 434.33.

TABLE 9
Compounds 11 and 11a.

| | | | Calc. | | Rt |
|---|---|---|---|---|---|
| No. | Structure | Exact Mass | [M + H]+ | m/z | (min) |
| 11 | 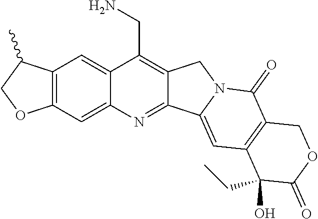 [SMILES: CC[C@@]1(O)C(=O)OCc2c1cc1n(c2=O)Cc2c-1nc1cc3c(cc1c2CN)C(C)CO3] | 433.16 | 434.17 | 434.25 | 1.04 |
| 11a | 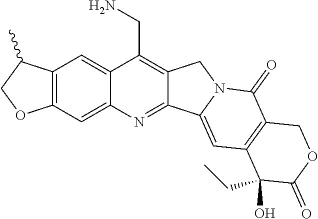 [SMILES: CC[C@@]1(O)C(=O)OCc2c1cc1n(c2=O)Cc2c-1nc1cc3c(cc1c2CN)C(C)CO3] | 433.16 | 434.17 | 434.33 | 1.03 |

### Example 12: Preparation of Compounds 12 and 12a-12b

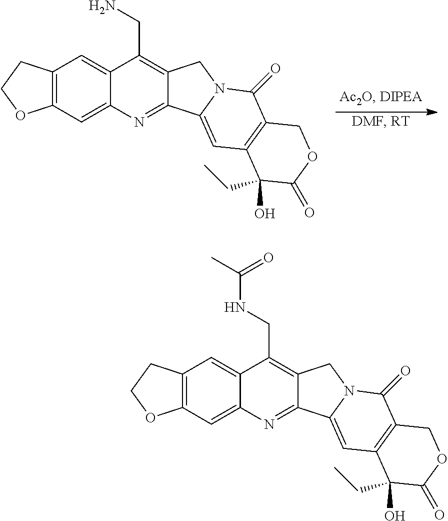

In a 4 mL vial equipped with a stir bar, (S)-5-(aminomethyl)-12-ethyl-12-hydroxy-2,3,9,12-tetrahydro-8H-furo[3,2-g]pyrano[3′,4′:6,7]indolizino[1,2-b]quinoline-8,11(6H)-dione (10 mg, 0.02 mmol) in DMF (0.5 mL). Acetic anhydride (2.9 mg, 0.03 mmol) and DIPEA (6.2 uL, 0.04 mmol) were added to the vial and reaction was stirred at RT for 2 h. Crude material was purified by preparative HPLC to yield product Compound 12 (1.7 mg, 15% yield).

General Method UPLC-MS: t_{R}=1.25 min, m/z (ES+) 462.17 (M+H)⁺, found 462.13.

TABLE 10
Compounds 12a-12b were made following the procedures outlined for
Compound 12.

| | | | Calc. | | Rt |
|---|---|---|---|---|---|
| No. | Structure | Exact Mass | [M + H]+ | m/z | (min) |
| 12a | 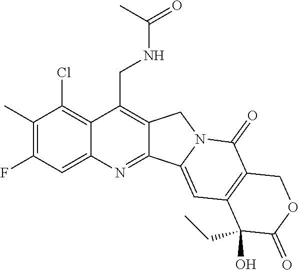 [SMILES: CC[C@@]1(O)C(=O)OCc2c1cc1n(c2=O)Cc2c-1nc1cc(F)c(C)c(Cl)c1c2CNC(C)=O] | 485.12 | 486.13 | 486.43 | 1.58 |
| 12b | 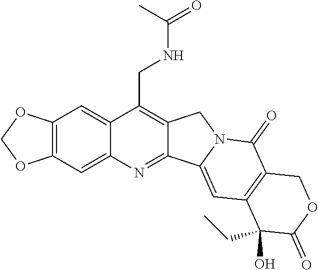 [SMILES: CC[C@@]1(O)C(=O)OCc2c1cc1n(c2=O)Cc2c-1nc1cc3c(cc1c2CNC(C)=O)OCO3] | 463.14 | 464.15 | 464.54 | 1.08 |

### Example 13: Preparation of Compounds 13 and 13a-13b

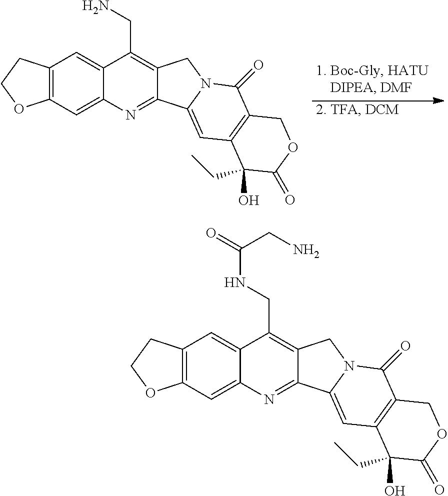

In a 4 mL vial equipped with a stir bar, dissolve Boc-Gly (4.2 mg, 0.02 mmol) in DMF (0.5 mL). Add HATU (7.9 mg, 0.02 mmol) and DIPEA (5.7 uL, 0.04 mmol) to the vial and let stir for 20 minutes. Add (S)-5-(aminomethyl)-12-ethyl-12-hydroxy-2,3,9,12-tetrahydro-8H-furo[3,2-g]pyrano[3′,4′:6,7]indolizino[1,2-b]quinoline-8,11(6H)-dione (9.2 mg, 0.02 mmol) to the vial and let reaction stir for 3 hours. Confirm coupling by UPLC-MS.

Remove DMF in vacuo and dissolve residue in 20% TFA in DCM and let stir for 1 hour. Crude was purified by preparatory HPLC to afford product Compound 13 (2.4 mg, 23% yield -2 steps).

General Method UPLC-MS: t_{R}=1.03 min. m/z (ES+) 477.17 (M+H)⁺, found 477.51.

TABLE 11
Compounds 13a-13b were made following the procedures outlined for
Compound 13.

| | | | Calc. | | Rt |
|---|---|---|---|---|---|
| No. | Structure | Exact Mass | [M + H]+ | m/z | (min) |
| 13a | 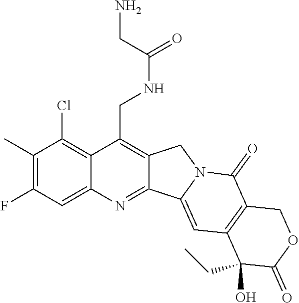 [SMILES: CC[C@@]1(O)C(=O)OCc2c1cc1n(c2=O)Cc2c-1nc1cc(F)c(C)c(Cl)c1c2CNC(=O)CN] | 500.13 | 501.14 | 501.23 | 1.20 |
| 13b | 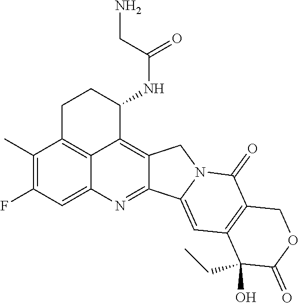 [SMILES: CC[C@@]1(O)C(=O)OCc2c1cc1n(c2=O)Cc2c-1nc1cc(F)c(C)c3c1c2[C@@H](NC(=O)CN)CC3] | 492.18 | 493.19 | 493.17 | 1.15 |

### Example 14: Preparation of Camptothecin Drug Linker Compounds

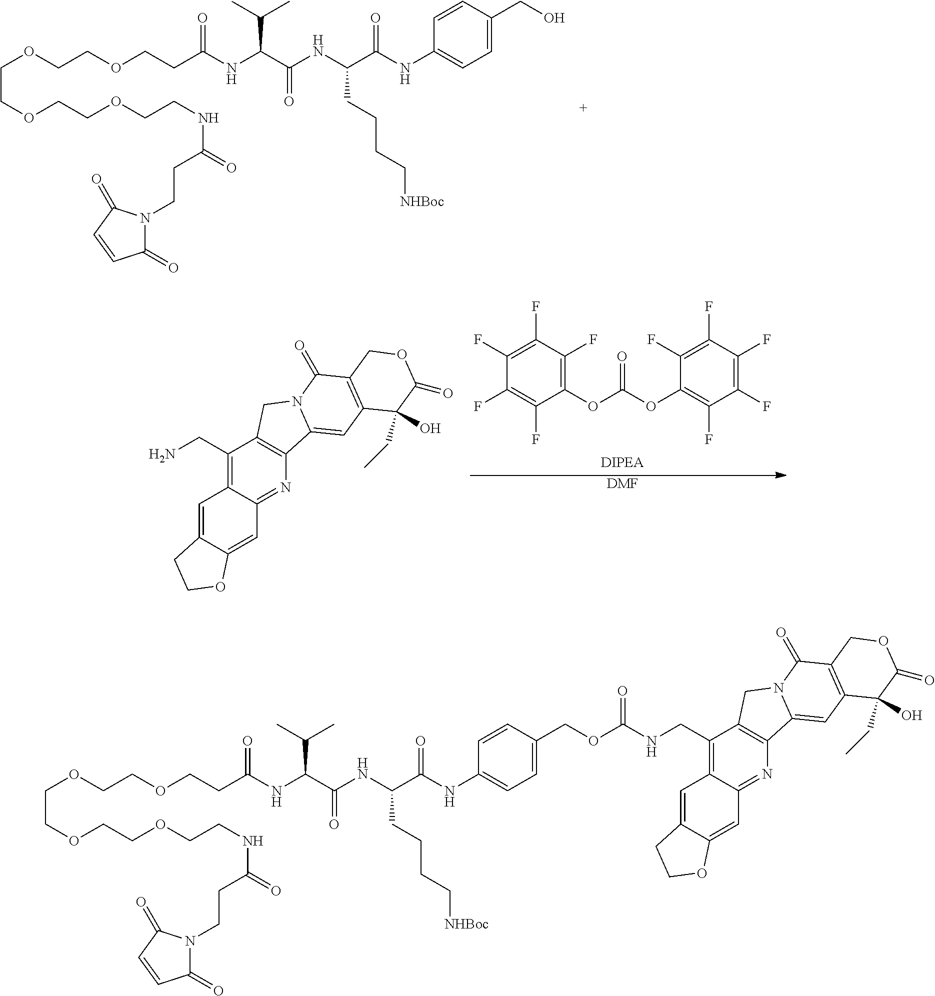

tert-butyl N-[(5S)-5-[[(2S)-2-[3-[2-[2-[2-[2-[3-(2,5-dioxopyrrol-1-yl)propanoylamino]ethoxy]ethoxy]ethoxy]ethoxy]propanoylamino]-3-methyl-butanoyl]amino]-6-[4-(hydroxymethyl)anilino]-6-oxo-hexyl]carbamate (1.25 eq, 76 mg, 0.0900 mmol), prepared according to the procedure reported in WO2019195665, was dissolved in DMF (1 mL). Bis(pentafluorophenyl) carbonate (1.50 eq, 43 mg, 0.108 mmol) was added to the reaction followed by N,N-Diisopropylethylamine (2.00 eq, 0.025 mL, 0.144 mmol). Complete conversion to the activated PFP intermediate was observed by UPLC-MS after 5 minutes. (5S)-14-(aminomethyl)-5-ethyl-5-hydroxy-7,20-dioxa-11,24-diazahexacyclo[11.11.0.02,11.04,9.015,23.017,21]tetracosa-1(24),2,4(9),13,15,17(21),22-heptaene-6,10-dione (1.00 eq, 30 mg, 0.0720 mmol) was added to the reaction and stirred for minutes at which point comlete conversion was observed by UPLC-MS. The reaction was acidified with AcOH (30 uL) and purified by Prep-HPLC 21×250 mm MaxRP 30-50-95% MeCN in H₂O 0.1% FA. Fractions containing the desired product were concentrated in vacuo to afford a yellow solid tert-butyl N-[(5S)-5-[[(2S)-2-[3-[2-[2-[2-[2-[3-(2,5-dioxopyirol-1-yl)propanoylamino]ethoxy]ethoxy]ethoxy]ethoxy]propanoylamino]-3-methyl-butanoyl]amino]-6-[4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]anilino]-6-oxo-hexyl]carbamate (35 mg, 0.0269 mmol, 37.34% yield). Rt=1.58 min General Method UPLC. MS (m/z) [M+H]⁺ calc. for C₆₅H₈₄N₉O₁₉ 1294.59, found 1294.52.

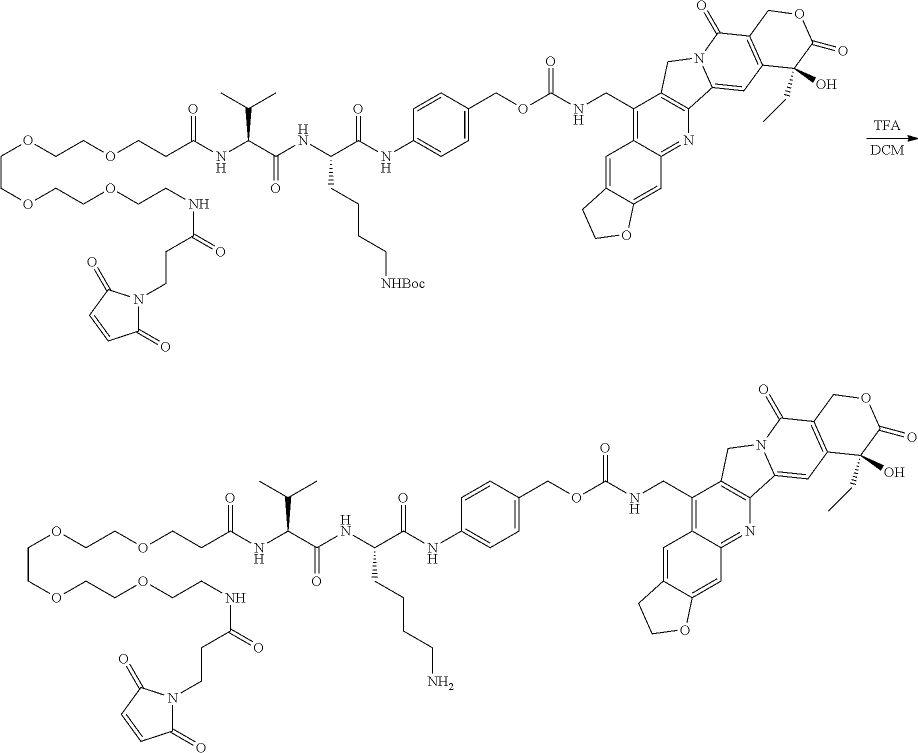 [SMILES: CC[C@@]1(O)C(=O)OCc2c1cc1n(c2=O)Cc2c-1nc1cc3c(cc1c2CNC(=O)OCc1ccc(NC(=O)[C@H](CCCCNC(=O)OC(C)(C)C)NC(=O)[C@@H](NC(=O)CCOCCOCCOCCOCCNC(=O)CCN2C(=O)C=CC2=O)C(C)C)cc1)CCO3 ; CC[C@@]1(O)C(=O)OCc2c1cc1n(c2=O)Cc2c-1nc1cc3c(cc1c2CNC(=O)OCc1ccc(NC(=O)[C@H](CCCCN)NC(=O)[C@@H](NC(=O)CCOCCOCCOCCOCCNC(=O)CCN2C(=O)C=CC2=O)C(C)C)cc1)CCO3]

tert-butyl N-[(5S)-5-[[(2S)-2-[3-[2-[2-[2-[2-[3-(2,5-dioxopyrrol-1-yl)propanoylamino]ethoxy]ethoxy]ethoxy]ethoxy]propanoylamino]-3-methyl-butanoyl]amino]-6-[4-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methylcarbamoyloxymethyl]anilino]-6-oxo-hexyl]carbamate (1.00 eq, 35 mg, 0.0269 mmol) was dissolved in 10% DCM (2 mL) in TFA. Stirred for 30 minutes and concentrated in vacuo. Complete conversion observed by UPLC-MS. The reaction was purified by prep-HPLC 21×250 mm MaxRP 20-35-95% MCCN in H₂O 0.05% TFA. Fractions containing the desired product were concentrated in vacuo to afford a yellow solid [4-[[(2S)-6-amino-2-[[(2S)-2-[3-[2-[2-[2-[2-[3-(2,5-dioxopyrrol-1-yl)propanoylamino]ethoxy]ethoxy]ethoxy]ethoxy]propanoylamino]-3-methyl-butanoyl]amino]hexanoyl]amino]phenyl]methyl N-[[(5S)-5-ethyl-5-hydroxy-6,10-dioxo-7,20-dioxa-11,24-diazahexacyclo[11.11.0.02,11.04,9.015,23.017,21]tetracosa-1(13),2,4(9),14,16,21,23-heptaen-14-yl]methyl]carbamate;2,2,2-trifluoroacetic acid Compound 14 (16 mg, 0.0120 mmol, 44.64% yield). Rt=1.22 min General Method UPLC. MS (m/z) [M+H]⁺ calc. for C₆₀H₇₆N₉O₁₇ 1194.54, found 1194.60.

TABLE 12
Compound 14a was made following the general procedures outlined for Compound 14.
Compound 14b was made following the general procedures outline for Compound 14.

| | | | | | Camptothecin |
|---|---|---|---|---|---|
| No. | Z′-A | S* or L^{P}(S*) | RL | Y | (N-link) |
| 14 | Mal—CH₂CH₂C(O)— | —NH(CH₂CH₂O)₄— | Val-Lys | PABC | Compound 2n |
| | | CH₂CH₂C(O)— | | | |
| 14a | Mal—CH₂CH₂C(O)— | —NH(CH₂CH₂O)₄— | Val-Lys | PABC | Compound 1 |
| | | CH₂CH₂C(O)— | | | |
| 4z | Mal—CH₂CH₂—C(O) | —NH((CH₂)₅—NH— | Val-Cit | PABC | Compound 2n |
| | | C(O)(CH₂CH₂O)₁₂CH₃)C(O)— | | | |

The following example was prepared using similar procedures as described for Compound 14.

| Com- | | | | | |
|---|---|---|---|---|---|
| pound | | Exact | Calc. | | Rt |
| Number | STRUCTURE | Mass | [M + H]+ | m/z | (min) |
| 4z | 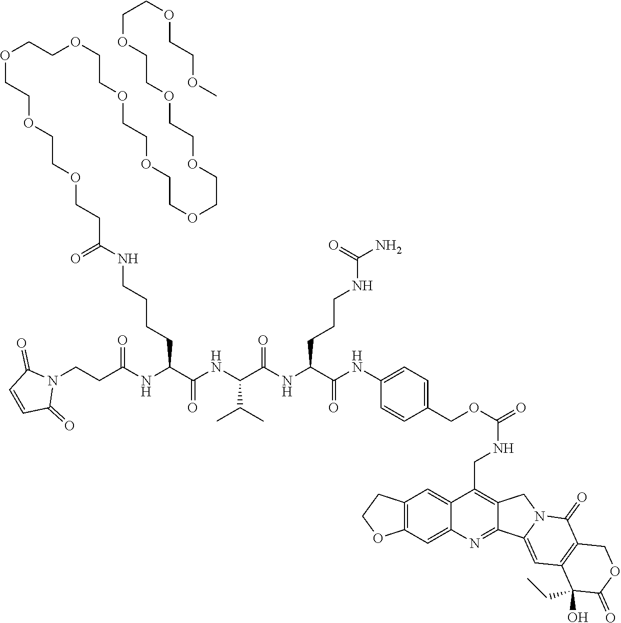 [SMILES: CC[C@@]1(O)C(=O)OCc2c1cc1n(c2=O)Cc2c-1nc1cc3c(cc1c2CNC(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOC)NC(=O)CCN2C(=O)C=CC2=O)C(C)C)cc1)CCO3] | 1673.80 | 1674.81 | 1675.73 | 1.51 |

### Characterization Data

| | | Calc'd MS | Observed MS | |
|---|---|---|---|---|
| No. | Parent Exact Mass | (m/z) [M + H]+ | (m/z) | RT |
| 14 | 1193.53 | 1194.54 | 1194.60 | 1.22 |
| 14a | 1195.51 | 1196.52 | 1196.23 | 1.22 |

### Aggregation Levels

TABLE 13
ADC aggregations levels for camptothecin drug-
linkers (DAR = 8). ADC aggregation was determined
by Size Exclusion Chromatography (SEC).

| | | | Conc. | |
|---|---|---|---|---|
| ADC | Description | DAR | (mg/mL) | HMW % |
| Ag1-Ex_14 | Glucuronide-Ex_2n | 8.2 | 1.15 | 11.64 |
| Ag4-Ex_14 | Glucuronide-Ex_2n | 8.2 | 0.99 | 9.05 |
| Ag1-Ex_14a | Glucuronide-Ex_2u | 8.5 | 0.94 | 22.44 |
| Ag4-Ex_14a | Glucuronide-Ex_2u | 8.4 | 1.00 | 16.49 |

### Example 15. Preparation of Compound 7

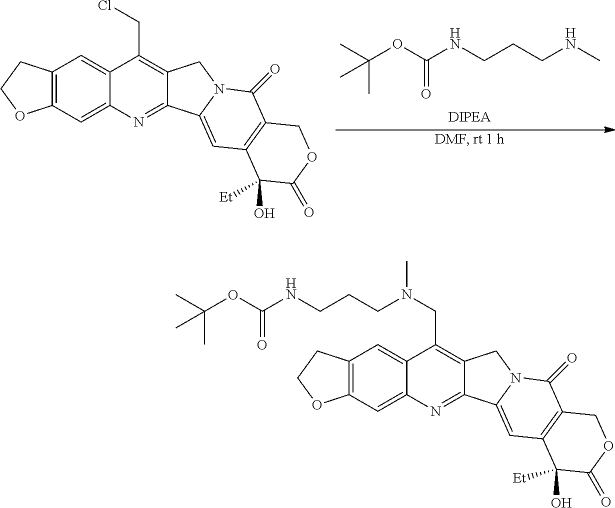 [SMILES: CC[C@@]1(O)C(=O)OCc2c1cc1n(c2=O)Cc2c-1nc1cc3c(cc1c2CCl)CCO3 ; CNCCCNC(=O)OC(C)(C)C ; "DMF, rt 1 h" ; CC[C@@]1(O)C(=O)OCc2c1cc1n(c2=O)Cc2c-1nc1cc3c(cc1c2CN(C)CCCNC(=O)OC(C)(C)C)CCO3]

rac-(5S)-14-(chloromethyl)-5-ethyl-5-hydroxy-7,18,20-trioxa-11,24-diazahexacyclo[11.11.0.02,11.04,9.015,23.017,21]tetracosa-1(13),2,4(9),14,16,21,23-heptaene-6,10-dione (1.00 eq, 50 mg, 0.113 mmol) was dissolved in DMF (1 mL) and added to tert-butyl N-[3-(methylamino)propyl]carbamate (3.00 eq, 64 mg, 0.340 mmol). Lithium iodide (1.00 eq, 15 mg, 0.113 mmol) was added followed by N,N-Diisopropylethylamine (6.00 eq, 0.12 mL, 0.681 mmol). The reaction was stirred for 1h, and then acidified with AcOH (200 uL) and purified by prep-HPLC. Fractions containing the desired product were concentrated in vacuo to afford a yellow solid tert-butyl N-[3-[[(5S)-5-ethyl-5-hydroxy-6,10-dioxo-7,18,20-trioxa-11,24-diazahexacyclo[11.11.0.02,11.04,9.015,23.017,21]tetracosa-1(13),2,4(9),14,16,21,23-heptaen-14-yl]methyl-methyl-amino]propyl]carbamate Compound 7 (45.13 mg, 0.0762 mmol, 67.14% yield). Rt=1.18 min General Method UPLC. MS (m/z) [M+H]⁺ calc. for C₃₁H₃₇N₄O₈ 593.26, found 593.56.

TABLE 14
The compounds of Table 14 were made following the general
procedures outlined for Compound 7.

| Compound | | Calc. | | RT |
|---|---|---|---|---|
| # | Structure | [M + H]+ | m/z | (min) |
| 7a | 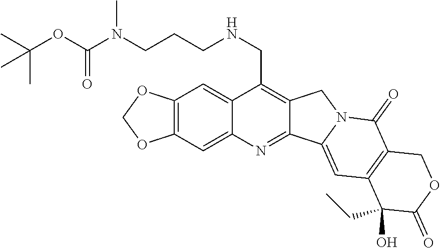 [SMILES: CC[C@@]1(O)C(=O)OCc2c1cc1n(c2=O)Cc2c-1nc1cc3c(cc1c2CNCCCN(C)C(=O)OC(C)(C)C)OCO3] | 593.26 | 593.46 | 1.31 |
| 7b | 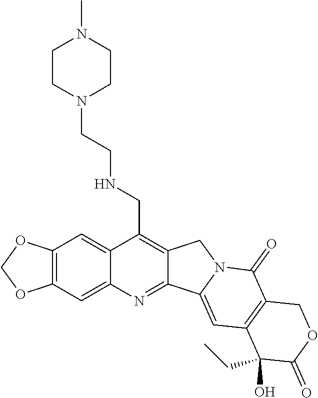 [SMILES: CC[C@@]1(O)C(=O)OCc2c1cc1n(c2=O)Cc2c-1nc1cc3c(cc1c2CNCCN1CCN(C)CC1)OCO3] | 548.25 | 548.55 | 1.07 |
| 7c | 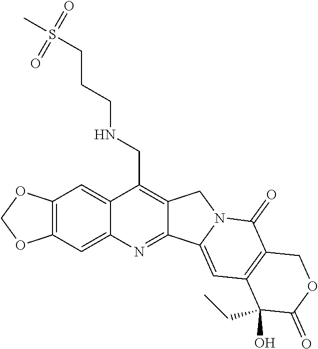 [SMILES: CC[C@@]1(O)C(=O)OCc2c1cc1n(c2=O)Cc2c-1nc1cc3c(cc1c2CNCCCS(C)(=O)=O)OCO3] | 542.16 | 542.54 | 0.88 |
| 7d | 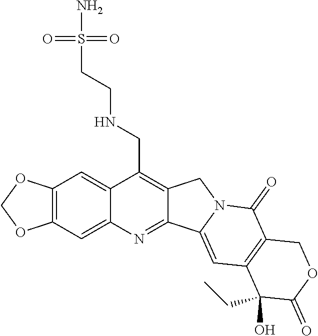 [SMILES: CC[C@@]1(O)C(=O)OCc2c1cc1n(c2=O)Cc2c-1nc1cc3c(cc1c2CNCCS(N)(=O)=O)OCO3] | 529.14 | 529.44 | 0.86 |
| 7e | 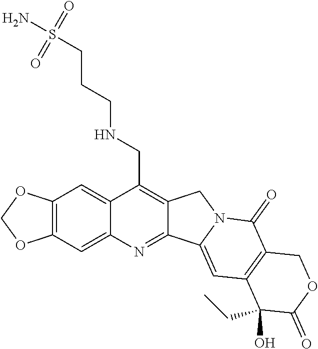 [SMILES: CC[C@@]1(O)C(=O)OCc2c1cc1n(c2=O)Cc2c-1nc1cc3c(cc1c2CNCCCS(N)(=O)=O)OCO3] | 543.16 | 543.12 | 0.87 |
| 7f | 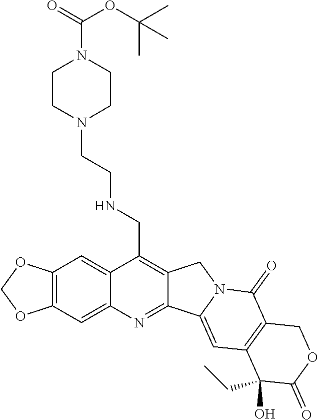 [SMILES: CC[C@@]1(O)C(=O)OCc2c1cc1n(c2=O)Cc2c-1nc1cc3c(cc1c2CNCCN1CCN(C(=O)OC(C)(C)C)CC1)OCO3] | 634.29 | 634.5 | 1.69 |
| 7g | 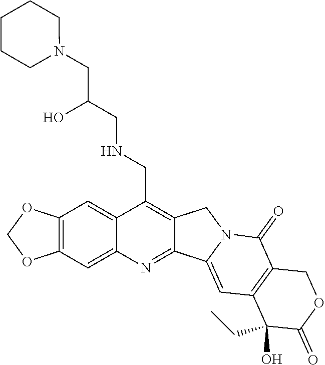 [SMILES: CC[C@@]1(O)C(=O)OCc2c1cc1n(c2=O)Cc2c-1nc1cc3c(cc1c2CNCC(O)CN1CCCCC1)OCO3] | 563.25 | 563.59 | 1.12 |
| 7h | 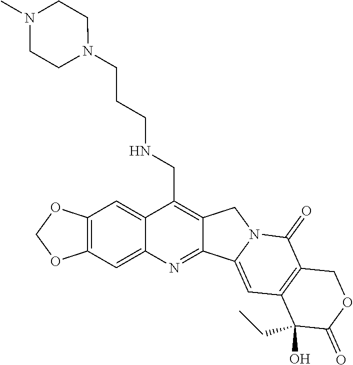 [SMILES: CC[C@@]1(O)C(=O)OCc2c1cc1n(c2=O)Cc2c-1nc1cc3c(cc1c2CNCCCN1CCN(C)CC1)OCO3] | 562.27 | 562.62 | 1.07 |
| 7i | 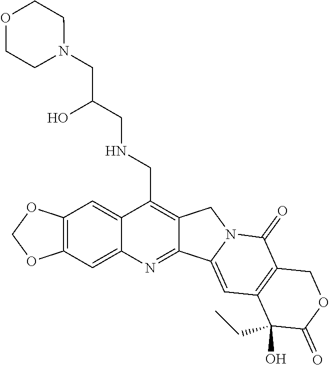 [SMILES: CC[C@@]1(O)C(=O)OCc2c1cc1n(c2=O)Cc2c-1nc1cc3c(cc1c2CNCC(O)CN1CCOCC1)OCO3] | 565.23 | 565.43 | 1.06 |
| 7j | 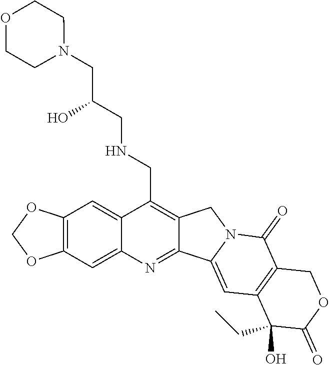 [SMILES: CC[C@@]1(O)C(=O)OCc2c1cc1n(c2=O)Cc2c-1nc1cc3c(cc1c2CNC[C@H](O)CN1CCOCC1)OCO3] | 565.23 | 565.43 | 1.07 |
| 7k | 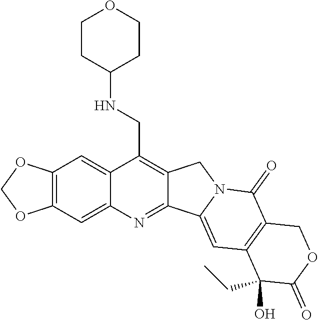 [SMILES: CC[C@@]1(O)C(=O)OCc2c1cc1n(c2=O)Cc2c-1nc1cc3c(cc1c2CNC1CCOCC1)OCO3] | 506.19 | 506.55 | 0.89 |
| 7l | 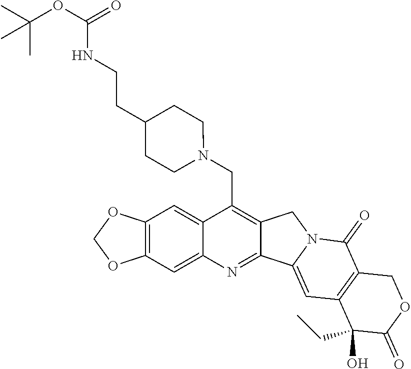 [SMILES: CC[C@@]1(O)C(=O)OCc2c1cc1n(c2=O)Cc2c-1nc1cc3c(cc1c2CN1CCC(CCNC(=O)OC(C)(C)C)CC1)OCO3] | | | |
| 7m | 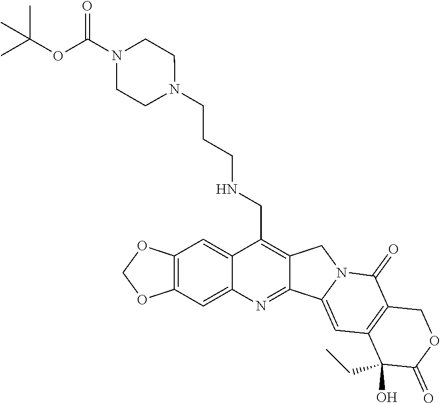 [SMILES: CC[C@@]1(O)C(=O)OCc2c1cc1n(c2=O)Cc2c-1nc1cc3c(cc1c2CNCCCN1CCN(C(=O)OC(C)(C)C)CC1)OCO3] | 648.31 | 648.66 | 1.34 |
| 7n | 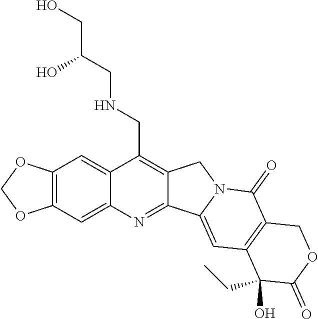 [SMILES: CC[C@@]1(O)C(=O)OCc2c1cc1n(c2=O)Cc2c-1nc1cc3c(cc1c2CNC[C@H](O)CO)OCO3] | 496.17 | 496.56 | 1.06 |
| 7o | 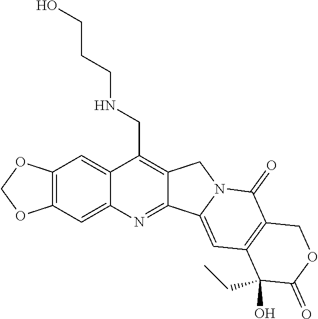 [SMILES: CC[C@@]1(O)C(=O)OCc2c1cc1n(c2=O)Cc2c-1nc1cc3c(cc1c2CNCCCO)OCO3] | 480.18 | 480.55 | 1.26 |
| 7p | 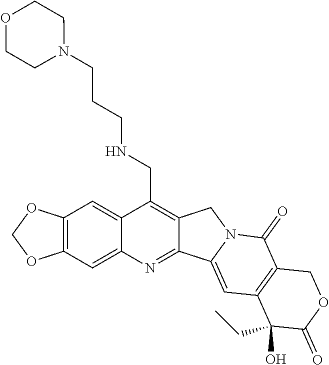 [SMILES: CC[C@@]1(O)C(=O)OCc2c1cc1n(c2=O)Cc2c-1nc1cc3c(cc1c2CNCCCN1CCOCC1)OCO3] | 549.24 | 549.52 | 1.09 |
| 7q | 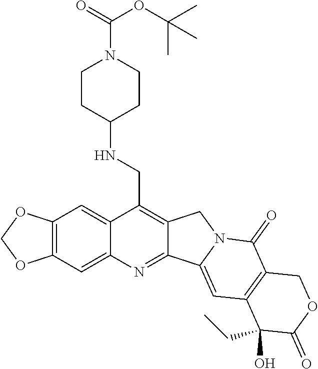 [SMILES: CC[C@@]1(O)C(=O)OCc2c1cc1n(c2=O)Cc2c-1nc1cc3c(cc1c2CNC1CCN(C(=O)OC(C)(C)C)CC1)OCO3] | 605.26 | 605.59 | 1.16 |
| 7r | 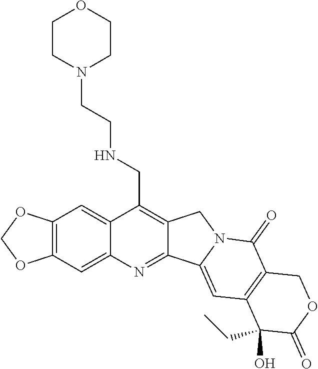 [SMILES: CC[C@@]1(O)C(=O)OCc2c1cc1n(c2=O)Cc2c-1nc1cc3c(cc1c2CNCCN1CCOCC1)OCO3] | 535.22 | 535.55 | 1.04 |
| 7s | 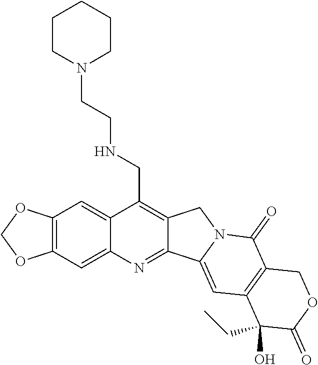 [SMILES: CC[C@@]1(O)C(=O)OCc2c1cc1n(c2=O)Cc2c-1nc1cc3c(cc1c2CNCCN1CCCCC1)OCO3] | 533.24 | 533.22 | 1.09 |
| 7t | 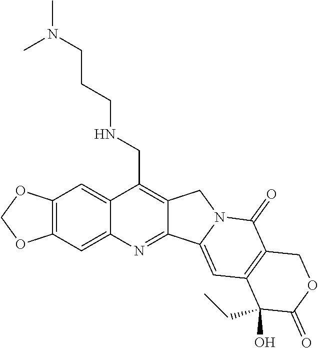 [SMILES: CC[C@@]1(O)C(=O)OCc2c1cc1n(c2=O)Cc2c-1nc1cc3c(cc1c2CNCCCN(C)C)OCO3] | 507.23 | 507.61 | 1.07 |
| 7u | 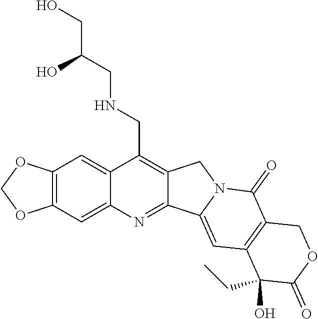 [SMILES: CC[C@@]1(O)C(=O)OCc2c1cc1n(c2=O)Cc2c-1nc1cc3c(cc1c2CNC[C@@H](O)CO)OCO3] | 496.17 | 496.46 | 1.12 |
| 7v | 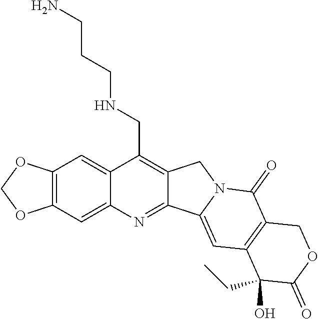 [SMILES: CC[C@@]1(O)C(=O)OCc2c1cc1n(c2=O)Cc2c-1nc1cc3c(cc1c2CNCCCN)OCO3] | 479.2 | 479.48 | 1.04 |
| 7w | 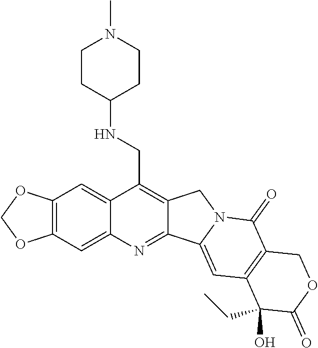 [SMILES: CC[C@@]1(O)C(=O)OCc2c1cc1n(c2=O)Cc2c-1nc1cc3c(cc1c2CNC1CCN(C)CC1)OCO3] | 519.23 | 519.74 | 1.09 |
| 7x | 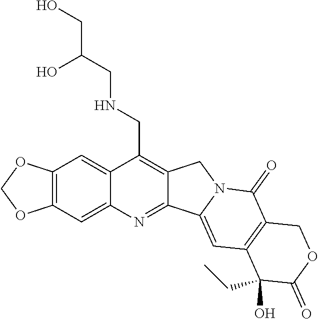 [SMILES: CC[C@@]1(O)C(=O)OCc2c1cc1n(c2=O)Cc2c-1nc1cc3c(cc1c2CNCC(O)CO)OCO3] | 496.17 | 496.46 | 1.13 |
| 7y | 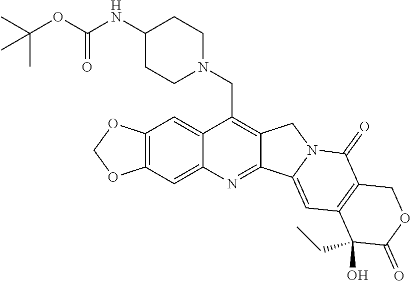 [SMILES: CC[C@@]1(O)C(=O)OCc2c1cc1n(c2=O)Cc2c-1nc1cc3c(cc1c2CN1CCC(NC(=O)OC(C)(C)C)CC1)OCO3] | 605.26 | 605.49 | 1.15 |
| 7z | 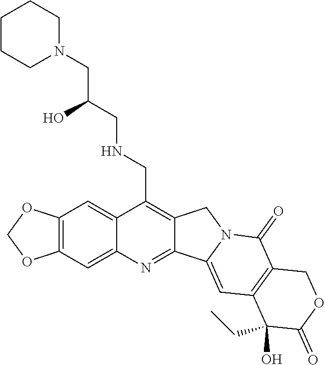 [SMILES: CC[C@@]1(O)C(=O)OCc2c1cc1n(c2=O)Cc2c-1nc1cc3c(cc1c2CNC[C@@H](O)CN1CCCCC1)OCO3] | 563.25 | 563.49 | 1.15 |
| 7aa | 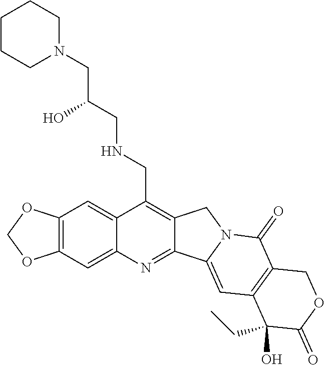 [SMILES: CC[C@@]1(O)C(=O)OCc2c1cc1n(c2=O)Cc2c-1nc1cc3c(cc1c2CNC[C@H](O)CN1CCCCC1)OCO3] | 563.25 | 563.68 | 1.14 |
| 7ab | 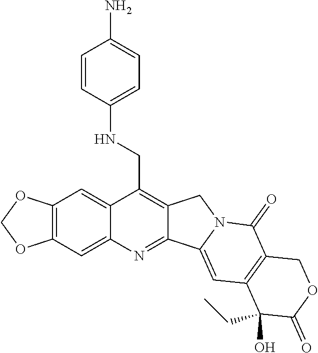 [SMILES: CC[C@@]1(O)C(=O)OCc2c1cc1n(c2=O)Cc2c-1nc1cc3c(cc1c2CNc1ccc(N)cc1)OCO3] | 513.18 | 513.08 | 1.08 |
| 7ac | 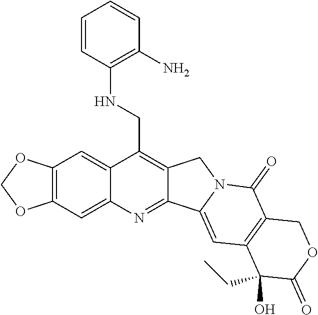 [SMILES: CC[C@@]1(O)C(=O)OCc2c1cc1n(c2=O)Cc2c-1nc1cc3c(cc1c2CNc1ccccc1N)OCO3] | 513.18 | 513.17 | 1.44 |
| 7ad | 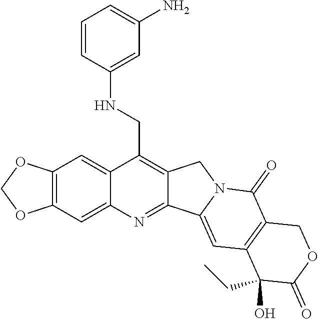 [SMILES: CC[C@@]1(O)C(=O)OCc2c1cc1n(c2=O)Cc2c-1nc1cc3c(cc1c2CNc1cccc(N)c1)OCO3] | 513.218 | 513.45 | 1.24 |
| 7ae | 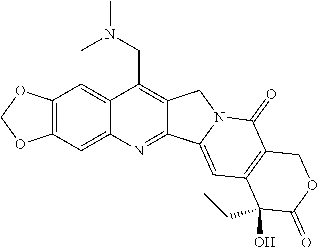 [SMILES: CC[C@@]1(O)C(=O)OCc2c1cc1n(c2=O)Cc2c-1nc1cc3c(cc1c2CN(C)C)OCO3] | 450.17 | 450.48 | 0.81 |
| 7af | 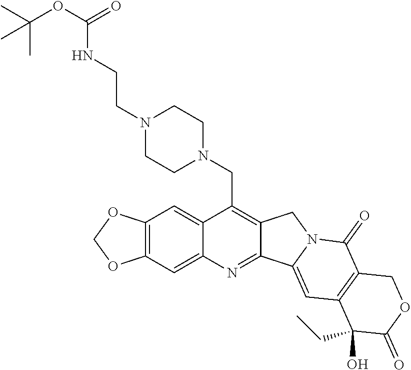 [SMILES: CC[C@@]1(O)C(=O)OCc2c1cc1n(c2=O)Cc2c-1nc1cc3c(cc1c2CN1CCN(CCNC(=O)OC(C)(C)C)CC1)OCO3] | 633.29 | 634.3 | 1.21 |
| 7ag | 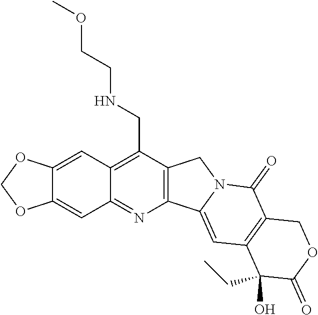 [SMILES: CC[C@@]1(O)C(=O)OCc2c1cc1n(c2=O)Cc2c-1nc1cc3c(cc1c2CNCCOC)OCO3] | 480.18 | 479.93 | 1.03 |
| 7ah | 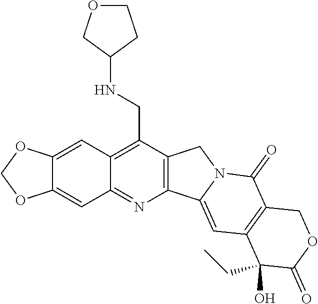 [SMILES: CC[C@@]1(O)C(=O)OCc2c1cc1n(c2=O)Cc2c-1nc1cc3c(cc1c2CNC1CCOC1)OCO3] | 492.18 | 491.96 | 0.99 |
| 7ai | 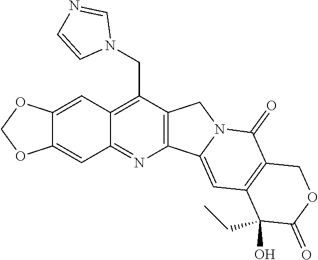 [SMILES: CC[C@@]1(O)C(=O)OCc2c1cc1n(c2=O)Cc2c-1nc1cc3c(cc1c2Cn1ccnc1)OCO3] | 473.15 | 473.25 | 0.99 |
| 7aj | 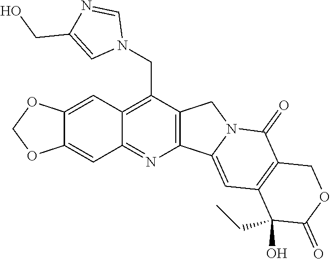 [SMILES: CC[C@@]1(O)C(=O)OCc2c1cc1n(c2=O)Cc2c-1nc1cc3c(cc1c2Cn1cnc(CO)c1)OCO3] | 203.16 | 203.24 | 0.98 |
| 7ak | 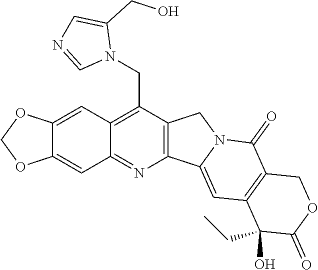 [SMILES: CC[C@@]1(O)C(=O)OCc2c1cc1n(c2=O)Cc2c-1nc1cc3c(cc1c2Cn1cncc1CO)OCO3] | 503.16 | 503.24 | 0.98 |
| 7al | 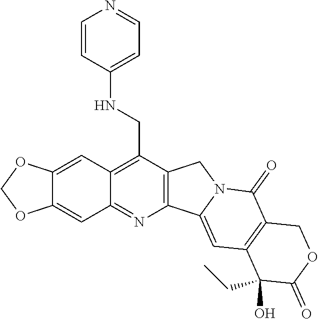 [SMILES: CC[C@@]1(O)C(=O)OCc2c1cc1n(c2=O)Cc2c-1nc1cc3c(cc1c2CNc1ccncc1)OCO3] | 499.16 | 498.97 | 1.03 |
| 7am | 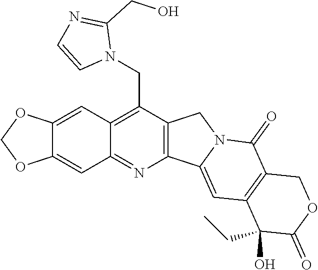 [SMILES: CC[C@@]1(O)C(=O)OCc2c1cc1n(c2=O)Cc2c-1nc1cc3c(cc1c2Cn1ccnc1CO)OCO3] | 503.16 | 503.14 | 0.98 |
| 7an | 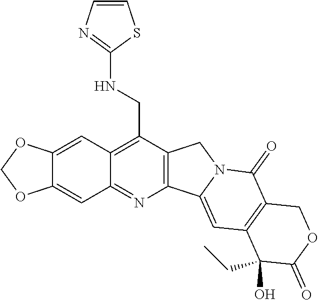 [SMILES: CC[C@@]1(O)C(=O)OCc2c1cc1n(c2=O)Cc2c-1nc1cc3c(cc1c2CNc1nccs1)OCO3] | 504.12 | 505.32 | 0.99 |

### Example 16. Preparation of Compounds 8a-8f

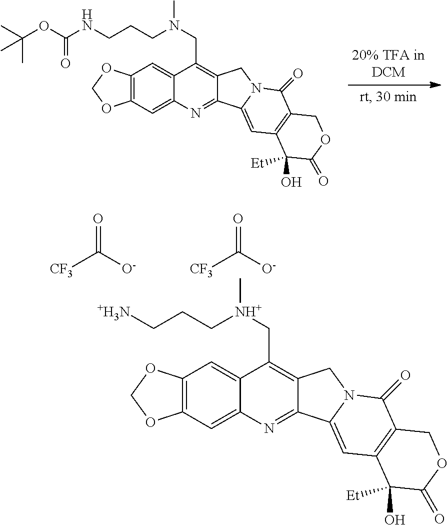 [SMILES: CC[C@@]1(O)C(=O)OCc2c1cc1n(c2=O)Cc2c-1nc1cc3c(cc1c2CN(C)CCCNC(=O)OC(C)(C)C)OCO3 ; O=C([O-])C(F)(F)F ; O=C([O-])C(F)(F)F ; CC[C@@]1(O)C(=O)OCc2c1cc1n(c2=O)Cc2c-1nc1cc3c(cc1c2C[NH+](C)CCC[NH3+])OCO3]

tert-butyl N-[3-[[(5S)-5-ethyl-5-hydroxy-6,10-dioxo-7,18,20-trioxa-11,24-diazahexacyclo[11.11.0.02,11.04,9.015,23.017,21]tetracosa-1(13),2,4(9),14,16,21,23-heptaen-14-yl]methyl-methyl-amino]propyl]carbamate (1.00 eq, 45 mg, 0.0762 mmol) was dissolved in 20% TFA in DCM (1 mL). The reaction was stirred for 30 minutes at which point complete conversion was observed. The reaction was concentrated in vacuo and purified by prep-HPLC. Fractions containing the desired product were concentrated in vacuo to afford a yellow solid (5S)-14-[[3-aminopropyl(methyl)amino]methyl]-5-ethyl-5-hydroxy-7,18,20-trioxa-11,24-diazahexacyclo[11.11.0.02,11.04,9.015,23.017,21]tetracosa-1(13),2,4(9),14,16,21,23-heptaene-6,10-dione;2,2,2-trifluoroacetic acid Compound 8 (42 mg, 0.0580 mmol, 76.18% yield). Rt=0.58 min General Method UPLC. MS (m/z) [M+H]⁺ calc. for C₂₆H₂₉N₄O₆ 493.21, found 493.55.

TABLE 15
Camptothecin derivatives prepared following the general procedures
outlined for Compound 8.

| Compound | | Calc. | | RT |
|---|---|---|---|---|
| # | Structure | [M + H]+ | m/z | (min) |
| 8a | 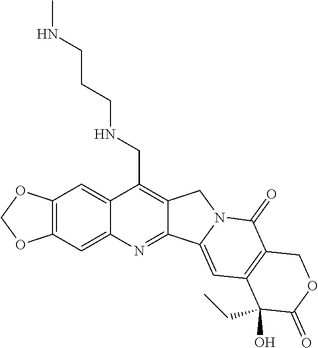 [SMILES: CC[C@@]1(O)C(=O)OCc2c1cc1n(c2=O)Cc2c-1nc1cc3c(cc1c2CNCCCNC)OCO3] | 493.21 | 493.65 | 0.83 |
| 8b | 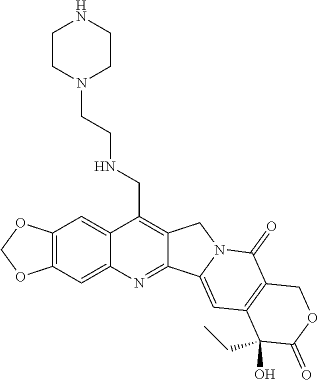 [SMILES: CC[C@@]1(O)C(=O)OCc2c1cc1n(c2=O)Cc2c-1nc1cc3c(cc1c2CNCCN1CCNCC1)OCO3] | 534.24 | 534.39 | 0.88 |
| 8c | 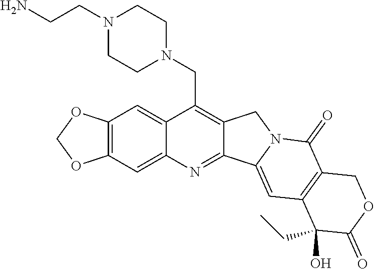 [SMILES: CC[C@@]1(O)C(=O)OCc2c1cc1n(c2=O)Cc2c-1nc1cc3c(cc1c2CN1CCN(CCN)CC1)OCO3] | 534.24 | 534.58 | 0.92 |
| 8d | 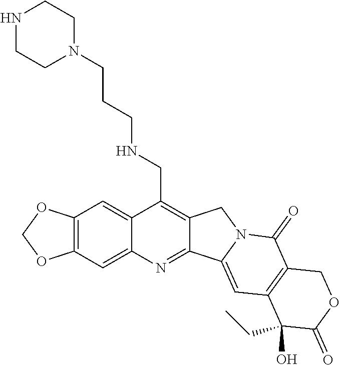 [SMILES: CC[C@@]1(O)C(=O)OCc2c1cc1n(c2=O)Cc2c-1nc1cc3c(cc1c2CNCCCN1CCNCC1)OCO3] | 548.25 | 548.55 | 1.05 |
| 8e | 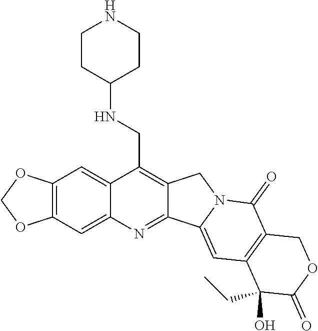 [SMILES: CC[C@@]1(O)C(=O)OCc2c1cc1n(c2=O)Cc2c-1nc1cc3c(cc1c2CNC1CCNCC1)OCO3] | 505.21 | 505.58 | 0.78 |
| 8f | 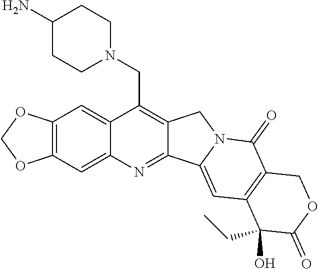 [SMILES: CC[C@@]1(O)C(=O)OCc2c1cc1n(c2=O)Cc2c-1nc1cc3c(cc1c2CN1CCC(N)CC1)OCO3] | 505.21 | 505.58 | 0.87 |

### Camptothecin Conjugation Method

Fully or partially reduced ADC8 were prepared in 50% propylene glycol (PG) IX PBS mixture. A half portion of the PG was added to reduced mAb, and half PG was added to the 1 mM DMSO camptothecin drug-linker stock. The PG/drug-linker mix was added to reduced mAb in 25% portions. After the addition of drug-linker was complete, excess drug-linker was removed by treating with activated charcoal (1 mg of charcoal to 1 mg of mAb). The charcoal was then removed via filtration, and the resulting ADC was buffer exchanged using a NAPS or PD 10 column, into 1X PBS pH 7.4.

### Example 17. Preparation of Compound 35

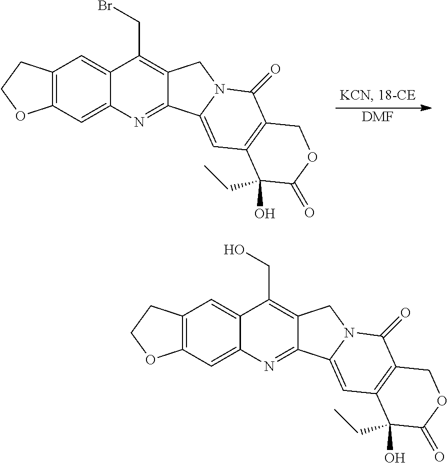

In a 4 mL vial equipped with a stir bar, (S)-5-(bromomethyl)-12-ethyl-12-hydroxy-2,3,9,12-tetrahydro-8H-furo[3,2-g]pyrano[3′,4′:6,7]indolizino[1,2-b]quinoline-8,11(6H)-dione (30 mg, 0.07 mmol) was dissolved in DMF (0.5 mL). KCN (8.9 mg, 0.14 mmol) and crown ether (18.1 mg, 0.07 mmol) were added to the reaction, and the reaction was stirred for 2 h at RT. Confirm reaction by UPLC-MS and remove solvent in vacuo. Product was purified by preparatory HPLC to afford product compound 35 (3.7 mg, 13% yield). General Method UPLC-MS: Rₜ=1.29 min. m/z (ES+) 421.14 (M+H)⁺, found 421.42.

TABLE 16
Camptothecin derivatives prepared following the general procedures
outlined for Compound 35.

| | | Calc. | | RT |
|---|---|---|---|---|
| Compound # | Structure | [M + H]+ | m/z | (min) |
| 35 | 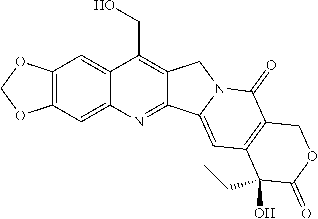 [SMILES: CC[C@@]1(O)C(=O)OCc2c1cc1n(c2=O)Cc2c-1nc1cc3c(cc1c2CO)OCO3] | 421.14 | 421.42 | 1.29 |

### Example 18. Preparation of Compound 15c

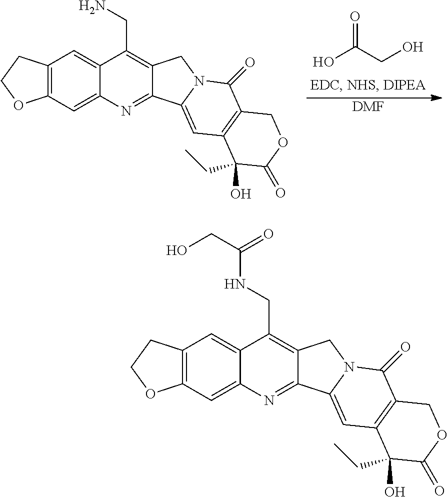

Glycolic acid (12.51 mg, 0.1645 mmoL) was dissolved in DMF (1 mL). N-hydroxysuccinimide (18.18 mg, 0.1579 mmol) was added followed by 1-Ethyl-3-(3-dimethylaminopropyl)carbodiimide hydrochloride (37.84 mg, 0.1974 mmol). The reaction was stirred for 20 minutes and then the solution was added to a vial containing (5S)-14-(aminomethyl)-5-ethyl-5-hydroxy-7,20-dioxa-11,24-diazahexacyclo[11.11.0.02,11.04,9.015,23.017,21]tetracosa-1(24),2,4(9),13,15,17(21),22-heptaene-6,10-dione (46 mg, 0.1097 mmol). DIPEA (38.2 μL, 0.219 mmol) was added to the reaction mixture and stirred for 5 minutes at which point complete conversion to the desired product was observed by UPLC-MS. The reaction was acidified with AcOH (50 μL) and purified by prep-HPLC 30×250 mm Max-RP, 10-35-95% MeCN in H₂O 0.05% TFA. Fractions containing the desired product were concentrated in vacuo to afford a yellow solid (S)-N-((12-ethyl-12-hydroxy-8,11-dioxo-2,3,6,9,11,12-hexahydro-8H-furo [3,2 g]pyrano[3′,4′:6,7]indolizino[1,2-b]quinolin-5-yl)methyl)-2-hydroxyacetamide (Compound 15c, 24.1 mg, 0.0505 mmol, 46.0%). General Method UPLC-MS: Rₜ=1.24 min. m/z (ES+) 478.16 (M+H)⁺, found 478.27.

TABLE 17
Camptothecin derivatives prepared following the general procedures
outlined for Compound 15c.

| Compound | | Calc. | | RT |
|---|---|---|---|---|
| # | Structure | [M + H]+ | m/z | (min) |
| 15a | 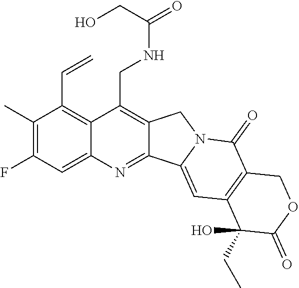 [SMILES: C=Cc1c(C)c(F)cc2nc3c(c(CNC(=O)CO)c12)Cn1c-3cc2c(c1=O)COC(=O)[C@]2(O)CC] | 494.17 | 493.95 | 1.40 |
| 15b | 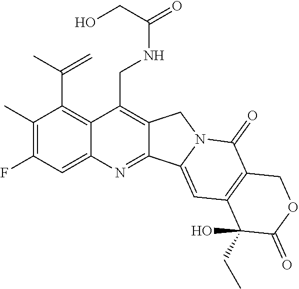 [SMILES: C=C(C)c1c(C)c(F)cc2nc3c(c(CNC(=O)CO)c12)Cn1c-3cc2c(c1=O)COC(=O)[C@]2(O)CC] | 508.19 | 508.23 | 1.61 |
| 15c | 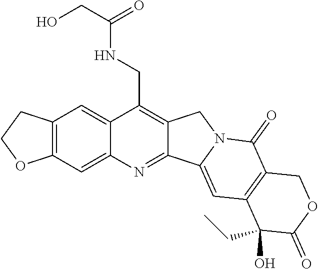 [SMILES: CC[C@@]1(O)C(=O)OCc2c1cc1n(c2=O)Cc2c-1nc1cc3c(cc1c2CNC(=O)CO)CCO3] | 478.16 | 478.27 | 1.24 |
| 15d | 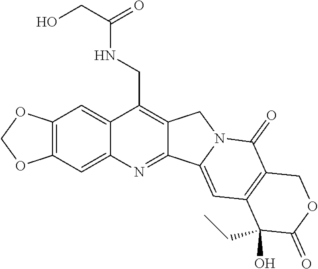 [SMILES: CC[C@@]1(O)C(=O)OCc2c1cc1n(c2=O)Cc2c-1nc1cc3c(cc1c2CNC(=O)CO)OCO3] | 480.14 | 480.07 | 1.25 |

### Example 18. Preparation of Compound 16

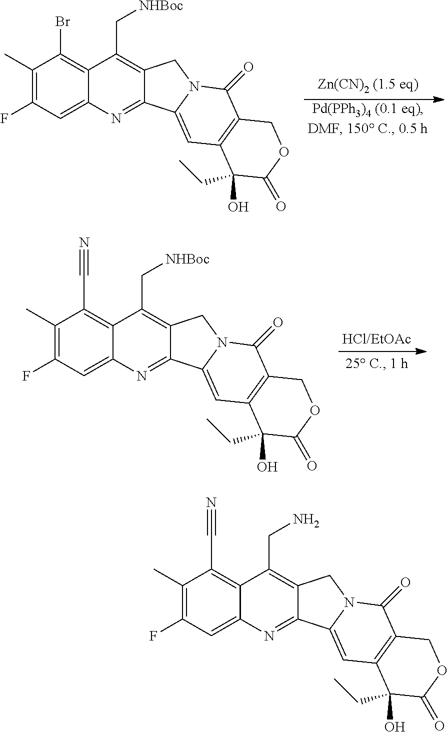 [SMILES: CC[C@@]1(O)C(=O)OCc2c1cc1n(c2=O)Cc2c-1nc1cc(F)c(C)c(Br)c1c2CNC(=O)OC(C)(C)C ; "Pd(PPh3)4 (0.1 eq),DMF, 150° C., 0.5 h" ; CC[C@@]1(O)C(=O)OCc2c1cc1n(c2=O)Cc2c-1nc1cc(F)c(C)c(C#N)c1c2CNC(=O)OC(C)(C)C ; CC[C@@]1(O)C(=O)OCc2c1cc1n(c2=O)Cc2c-1nc1cc(F)c(C)c(C#N)c1c2CN]

To a solution of CPT (100 mg, 170 umol) in DMF (5 mL) was added Pd(PPh₃)₄, (40 mg, 35 umol) and zinc cyanide (80 mg, 680 umol) at 20° C. in a microwave tube. The reaction was heated at 150° C. for 0.5 h under microwave. The resulting mixture was partitioned between ethyl acetate (100 mL) and water (200 mL), and then the aqueous phase was further extracted with ethyl acetate (3×30 mL). The organic phase was washed with brine, dried over Na₂SO₄, filtered and concentrated under reduced pressure to give a residue. The residue was purified by prep-HPLC (TFA condition) to give Boc-protected intermediate (12 mg, yield 13%).

A mixture of CPT intermediate (12 mg, 22.5 umol) in HCl/EtOAc (4 M, 1 mL) was stirred at 25° C. for 2 h. LCMS analysis showed reactant was consumed completely and one main peak with desired mass was detected. The resulting mixture was concentrated to give a residue which was purified by prep-HPLC (TFA condition) to give final product Compound 16 (5 mg, 41% yield). General Method UPLC-MS: Rₜ=1.07 min. m/z (ES+) 435.15 (M+H)⁺, found 435.29

TABLE 18
Camptothecin derivatives prepared following the general procedures
outlined for Compound 16.

| Compound | | Calc. | | RT |
|---|---|---|---|---|
| # | Structure | [M + H]+ | m/z | (min) |
| 16 | 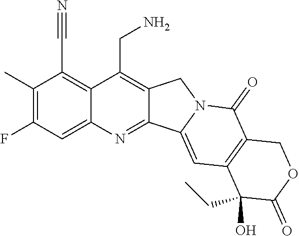 [SMILES: CC[C@@]1(O)C(=O)OCc2c1cc1n(c2=O)Cc2c-1nc1cc(F)c(C)c(C#N)c1c2CN] | 435.15 | 435.29 | 1.07 |

### Example 19. Preparation of Compound 17

To a solution of CPT (20 mg, 37.34 mmol) in H₂O (0.3 mL) and tert-butanol (1 mL) was added AD-mix-beta (58.1 mg, 74.6 mmol) at 0° C. The reaction mixture was warmed to 20° C. and stirred at 20° C. for 48 h. LCMS analysis showed starting material was consumed completely and one main peak with desired mass was detected. The resulting mixture was purified by pre-HPLC (TFA condition) to afford intermediate (5 mg, 12% yield).

A mixture of CPT intermediate (12 mg, 22.5 mmol) in HCl/EtOAc (4 M, 1 mL) was stirred at 25° C. for 2 h. LCMS analysis showed reactant was consumed completely and one main peak with desired mass was detected. The resulting mixture was concentrated to give a residue which was purified by reverse phase HPLC (9n) to afford product Compound 17 (5 mg, 41% yield). General Method UPLC-MS: t_{R}=1.71 min. m/z (ES+) 450.15 (M+H)⁺, found 450.00.

TABLE 19
Camptothecin derivatives prepared following the general procedures
outlined for Compound 17.

| Compound | | Calc. | | RT |
|---|---|---|---|---|
| # | Structure | [M + H]+ | m/z | (min) |
| 17 | 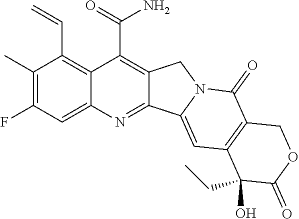 [SMILES: C=Cc1c(C)c(F)cc2nc3c(c(C(N)=O)c12)Cn1c-3cc2c(c1=O)COC(=O)[C@]2(O)CC] | 450.15 | 450.00 | 1.71 |

### Example 20. Preparation of Compound 18

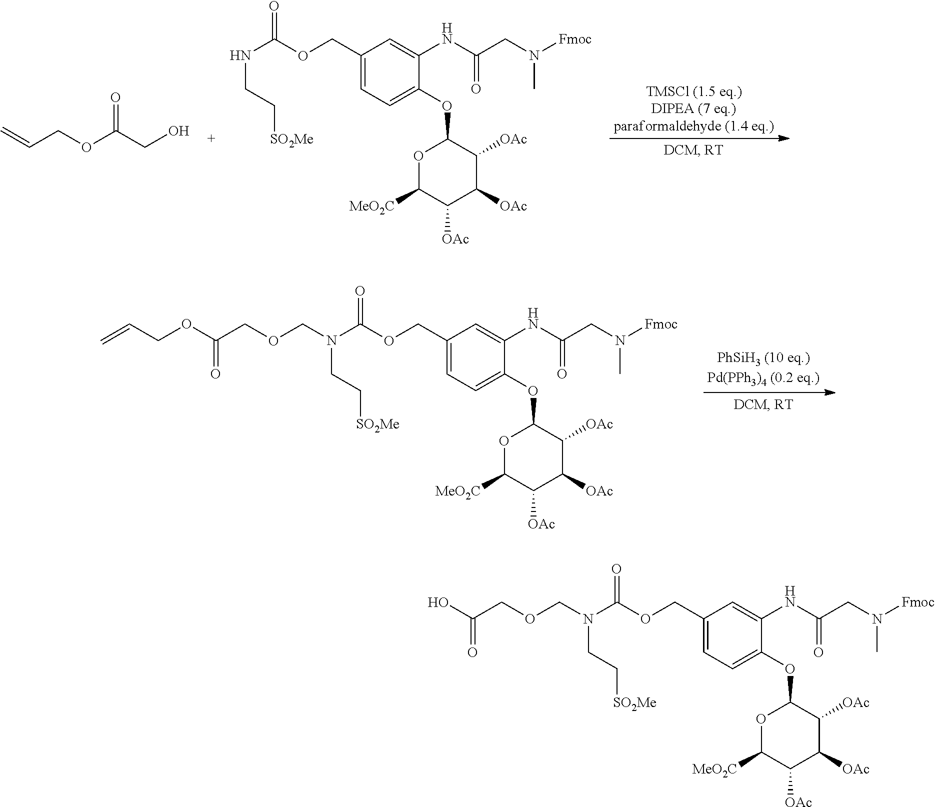

The ally ester of glycolic acid was made according to literature ACS Cent. Sci. 2020, 6, 2, 226.

In a scintillation vial equipped with stir bar, MAC linker precursor (450 mg, 0.5 mmol) was dissolved in DCM and added TMSCl (96 uL, 0.75 mmol) and paraformaldehyde (21 mg, 0.7 mmol) and stirred at room temp overnight. Solution was clear and DIPEA (323 mg, 2.5 mmol) was added to the reaction mixture. Glycol acid ally ester (290 uL, 2.5 mmol) was added to the reaction and stirred overnight. Reverse phase biotage (5-95% ACN:H₂O+0.5% formic acid) afforded the intermediate (225 mg, 45% y).

In scintillation vial equipped with stir bar, intermediate (225 mg, 0.22 mmol) was dissolved in DCM (0.5 mL) and THE (0.5 mL). PhSiH3 (237 mg, 2.2 mmol) was added followed by Pd(PPh₃)₄ (58 mg, 0.05 SGD-9493 mmol). Reaction was stirred for 2 h at 25° C. Reverse phase biotage (5-95% ACN:H₂O+0.5% formic acid) afforded the desired product Compound 18 (160 mg, 73% yield). General Method UPLC-MS: t_{R}=2.11 min. m/z (ES+) 986.26 (M+H)⁺, found 986.37.

### Example 20. Preparation of Compound 19

To a 4 mL scintillation vial equipped with stir bar was added carboxylate 2-(((((3-(2-((((9H-fluoren-9-yl)methoxy)carbonyl)(methyl)amino)acetamido)-4-(((2S,3R,4S,5S,6S)-3,4,5-triacetoxy-6-(methoxycarbonyl)tetrahydro-2H-pyran-2-yl)oxy)benzyl)oxy)carbonyl)(2-(methylsulfonyl)ethyl)amino)methoxy)acetic acid (19.8 mg, 0.02 mmol), HATU (7.6 mg, 0.02 mmol), DIPEA (7.7 μL, 0.06 mmol) and DMF (0.2 mL). The reaction was stirred for 10 min at 25° C. (S)-11-(aminomethyl)-4-ethyl-8-fluoro-4-hydroxy-9-methyl-10-vinyl-1,12-dihydro-14H-pyrano[3′,4′:6,7]indolizino[1,2-b]quinoline-3,14(4H)-dione (8.7 mg, 0.02 mmol) was added to the reaction and stirred for 3 h. Solvent was removed in vacuo purified by reverse phase HPLC (5-95% ACN:H₂O+0.5% formic acid) to afford the intermediate (9.8 mg, 70% yield).

To a 4 mL scintillation vial equipped with stir bar was added (2S,3R,4S,5S,6S)-2-(2-(2-((((9H-fluoren-9-yl)methoxy)carbonyl)(methyl)amino)acetamido)-4-(10-((S)-4-ethyl-8-fluoro-4-hydroxy-9-methyl-3,14-dioxo-10-vinyl-3,4,12,14-tetrahydro-1 H-pyrano[3′,4′:6,7]indolizino[1,2-b]quinolin-11-yl)-4-(2-(methylsulfonyl)ethyl)-3,8-dioxo-2,6-dioxa-4,9-diazadecyl)phenoxy)-6-(methoxycarbonyl)tetrahydro-2H-pyran-3,4,5-triyl triacetate (9.8 mg, 0.014 mmol), THE (0.2 mL), and McOH (0.2 mL). The reaction was cooled to −20° C. and stirred for 10 minutes. LiOH in water (420 uL, 84 mmol, 20 mM) was added to the reaction and stirred for 1 h at −20° C. and 6 h at 25° C. Solvent was removed in vacuo purified by reverse phase HPLC (5-95% ACN:H₂O+0.5% formic acid) to afford the intermediate (4.6 mg, 62% yield).

To a 4 mL scintillation vial equipped with stir bar was added (2S,3S,4S,5R,6S)-6-(4-(10-((S)-4-ethyl-8-fluoro-4-hydroxy-9-methyl-3,14-dioxo-10-vinyl-3,4,12,14-tetrahydro-1 H-pyrano[3′,4′:6,7]indolizino[1,2-b]quinolin-11-yl)-4-(2-(methylsulfonyl)ethyl)-3,8-dioxo-2,6-dioxa-4,9-diazadecyl)-2-(2-(methylamino)acetamido)phenoxy)-3,4,5-trihydroxytetrahydro-2H-pyran-2-carboxylic acid (1.27 mg, 0.001 mmol), DIPEA (0.43 uL, 0.002 mmol), and MP-OSu (0.39 mg, 0.002 mmol), and DMF (500 μL). The reaction was and stirred for 3 hours at room temperature. Solvent was removed in vacuo purified by reverse phase HPLC (5-95% ACN:H₂O+0.5% formic acid) to afford the product Compound 19 (0.70 mg, 48.13% yield). General Method UPLC-MS: t_{R}=1.45 min, m/z (ES+) 1192.35 (M+H)⁺, found 1191.96.

TABLE 20
Camptothecin derivatives prepared following the general procedures
outlined for Compound 19.

| Compound | | Calc. | | RT |
|---|---|---|---|---|
| # | Structure | [M + H]+ | m/z | (min) |
| 19 | 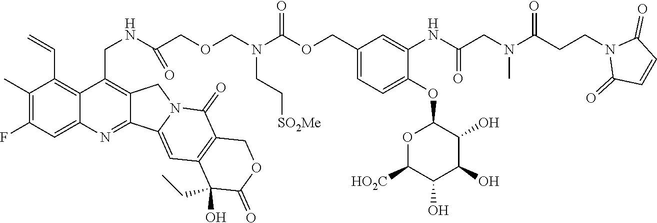 [SMILES: C=Cc1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] | 1192.35 | 1191.96 | 1.45 |
| 19a | 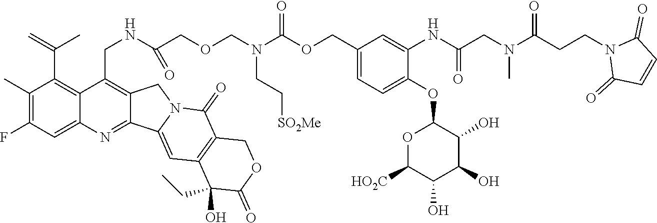 [SMILES: C=C(C)c1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC] | 1206.36 | 1206.98 | 1.50 |
| 19b | 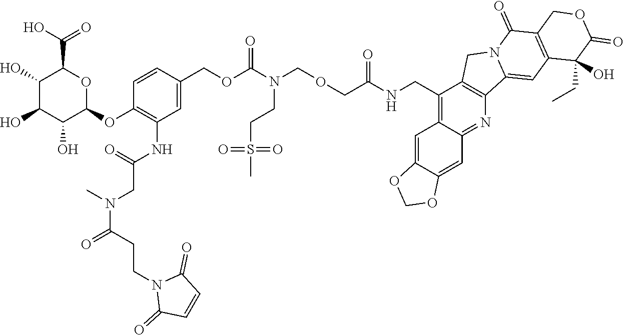 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)OCO3] | 1178.32 | 1178.87 | 1.29 |
| 19c | 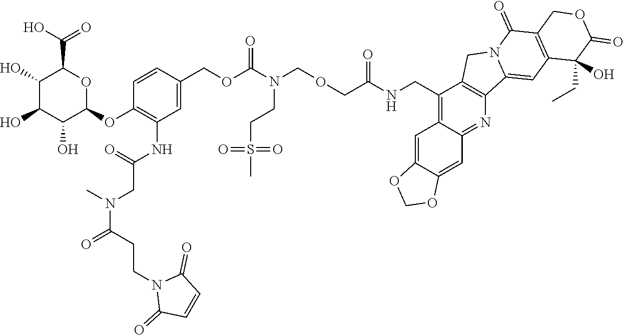 [SMILES: CC[C@@]1(O)C(=O)OCc2c1cc1n(c2=O)Cc2c-1nc1cc3c(cc1c2CNC(=O)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)OCO3] | 1176.34 | 1176.81 | 1.31 |

### Example 21. Preparation of Compound 21

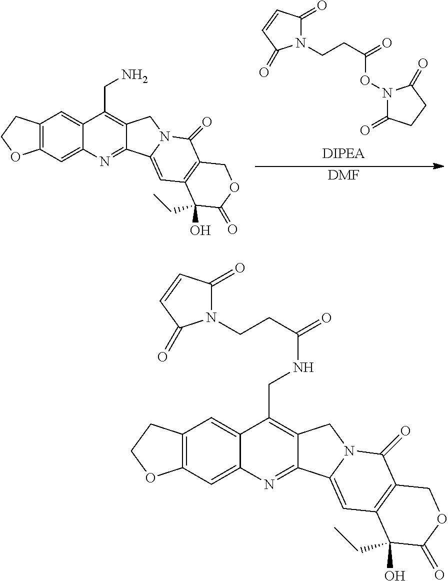

(5S)-14-(aminomethyl)-5-ethyl-5-hydroxy-7,20-dioxa-11,24-diazahexacyclo[11.11.0.02,11.04,9.015,23.017,21]tetracosa-1(24),2,4(9),13,15,17(21),22-heptaene-6,10-dione (3.0 mg, 0.0072 mmol) was dissolved in DMF (0.25 mL). 2,5-dioxopyirolidin-l-yl 3-(2,5-dioxo-2,5-dihydro-1H-pyrrol-1-yl)propanoate (2.3 mg, 0.0086 mmol) was added followed by DIPEA (2.5 μL, 0.014 mmol). Complete conversion was observed by UPLC-MS after 30 minutes. The reaction was acidified with AcOH (10 μL) and purified by prep-HPLC 10×250 mm Max-RP 5-60-95% MeCN in H₂O 0.05% TFA. Fractions containing the desired product were concentrated in vacuo to afford a yellow solid 3-(2,5-dioxopyirol-1-yl)-N-[[(5S)-5-ethyl-5-hydroxy-6,10-dioxo-7,20-dioxa-11,24-diazahexacyclo[11.11.0.02,11.04,9.015,23.017,21]tetracosa-1(24),2,4(9),13,15,17(21),22-heptaen-14-yl]methyl]propenamide (Compound 21, 1.91 mg, 0.0033 mmol, 46%). General Method UPLC-MS: t_{R}=1.33 min, m/z (ES+) 571.19 (M+H)⁺, found 570.95.

### Biological Examples

In vitro Small Molecule and ADC Evaluation

In vitro potency was assessed on multiple cancer cell lines. All cell lines were authenticated by STR profiling at IDEXX Bioresearch and cultured for no more than 2 months after resuscitation. Cells cultured in log-phase growth were seeded for 24 hours in 96-well plates containing 150 pi RPMI 1640 supplemented with 20% FBS. Serial dilutions of antibody-drug conjugates in cell culture media were prepared at 4x working concentrations, and 50 μL of each dilution was added to the 96-well plates. Following addition of test articles, cells were incubated with test articles for 4 days at 370° C. After 96 hours, growth inhibition was assessed by CellTiterGlo® (Promega, Madison, WI) and luminescence was measured on a plate reader. The IC₅₀ value, determined in triplicate, is defined here as the concentration that results in 50% reduction in cell growth relative to untreated controls.

In the following Tables IC₅₀ values for ADC8 and camptothecin free drugs are given in ng/mL and nmol/L concentrations, respectively, with values in the parenthesis representing percent cells remaining at highest concentration tested (1000 ng/mL for ADCs and 1 μM for camptothecin free compound, unless otherwise indicated) relative to untreated cells. Cell viability was determined by CellTiter-Glo staining after 96h exposure to ADC. ND=Not Determined. In the following tables, “Ex_” refers to a drug linker number. For example, “Ag4-Ex_4a” refers to a conjugate of an Ag4 antibody with drug linker 4a.

TABLE 21
In vitro potency (IC₅₀ values) of CPT free drugs (nmol/L) targeting
renal carcinoma cells (786-O), melanoma cells (A2058), pancreatic cancer cells (BxPC3),
non-small-cell lung cancer cells (Calu1), anaplastic large cell lymphoma cells (DEL,
DELBVR, Karpas299), Hodgkin's lymphoma cells (L540cy, Ls174T), breast cancer cells
(MDA-MB-231), acute myeloid leukemia cells (MOLM-13), and B-lymphocyte cancer cells
(SU-DHL4).

| | 786-O | A2058 | BxPC3 | Calu1 | DEL | DELBVR |
|---|---|---|---|---|---|---|
| Drug | IC₅₀ | IC₅₀ | IC₅₀ | IC₅₀ | IC₅₀ | IC₅₀ |
| 2n | 5 | 3 | 3 | 18 | 1 | 1 |
| 2ad | 9 | ND | 22 | ND | 1 | 1 |
| 2ah | 108 | ND | 199 | ND | 12 | 11 |
| 2ai | 64 | ND | 57 | ND | 6 | 5 |
| 2aj | 2 | ND | 3 | ND | 0.3 | 0.3 |
| 2ak | 57 | ND | 88 | ND | 6 | 6 |
| 2al | 4 | ND | 7 | ND | 1 | 1 |
| 2am | 2 | ND | 4 | ND | 0.3 | 0.3 |
| 2ap | 8 | ND | 14 | ND | 1 | 1 |
| 13a | 6 | ND | 5 | ND | 0.3 | 1 |
| 12a | 1 | ND | 2 | ND | 0.2 | 0.2 |
| 13 | 139 | ND | 94 | ND | 8 | 21 |
| 12 | 13 | ND | 12 | ND | 1 | 1 |
| 13b | 27 | ND | 21 | ND | 1 | 4 |
| 2aq | 11 | 18 | 27 | 1 | 2 | 3 |
| 10 | 54 | 33 | 64 | 95 | 7 | 15 |
| 14 | 4 | 2 | 6 | 9 | 0.6 | 1 |
| 5b | 69 | 17 | 128 | 54 | 17 | 15 |
| 2ar | 26 | 7 | 43 | 26 | 7 | 5 |
| 2as | 3 | 3 | 5 | 4 | 1 | 1 |
| 2at | 50 | 20 | 84 | 3' | 11 | 8 |
| 2au | 4 | 3 | 6 | 7 | 1 | 1 |
| 2av | 22 | 4 | 34 | 25 | 5 | 4 |
| 5c | 1 | 5 | 8 | 4 | 0.3 | |
| 5a | 56 | 16 | 154 | 389 | 7 | 10 |
| 5 | 16 | 7 | 40 | 129 | 4 | 4 |
| 2aw | 58 | 18 | 149 | 269 | 15 | 15 |
| 11 | 8 | 3 | 12 | 69 | 1 | 2 |
| 11a | 15 | 5 | 23 | 131 | 2 | 4 |
| 6 | 5 | 5 | 8 | 17 | 1 | 3 |
| 6a | 6 | 5 | 8 | 21 | 2 | 3 |
| 33 | 10 | 6 | 11 | 36 | 2 | 2 |
| 33a | 34 | 18 | 50 | 120 | 7 | 12 |
| 6b | 23 | 15 | 18 | 32 | 5 | 5 |
| 6c | 4 | 5 | 5 | 15 | 1 | 2 |
| 6d | 23 | 13 | 15 | 43 | 3 | 12 |
| 6g | 6 | 4 | 7 | 25 | 1 | 1 |
| 6f | 97 | 17 | 46 | 294 | 9 | 8 |
| 12b | 12 | ND | 11 | ND | 0 | 1 |
| 6e | 29 | 11 | 65 | 155 | 4 | 4 |
| 6h | 36 | 20 | 100 | 155 | 6 | 7 |
| 6i | 5 | 2 | 10 | 26 | 1 | 1 |

TABLE 22
(cont.) In vitro potency (IC₅₀ values) of CPT free drugs (nmol/L)
targeting renal carcinoma cells (786-O), melanoma cells (A2058), pancreatic cancer cells
(BxPC3), non-small-cell lung cancer cells (Calu1), anaplastic large cell lymphoma cells
(DEL, DELBVR, Karpas299), Hodgkin's lymphoma cells (L540cy, Ls174T), breast cancer
cells (MDA-MB-231), acute myeloid leukemia cells (MOLM-13), and B-lymphocyte cancer
cells (SU-DHL4).

| | Karpas299 | L540cy | Ls174T | MDAMB | MOLM- | SU-DHL- |
|---|---|---|---|---|---|---|
| Drug | IC₅₀ | IC₅₀ | IC₅₀ | 231 IC₅₀ | 13 IC₅₀ | 4 IC₅₀ |
| 2n | 5 | 2 | 4 | 8 | 29 | 1 |
| 2ad | 7 | 4 | 17 | 15 | 5 | 4 |
| 2ah | 65 | 54 | 152 | 149 | 125 | 29 |
| 2ai | 47 | 47 | 64 | 79 | 34 | 13 |
| 2aj | 2 | 1 | 5 | 5 | 1 | 1 |
| 2ak | 40 | 22 | 50 | 147 | 129 | 11 |
| 2al | 4 | 2 | 11 | 9 | 2 | 1 |
| 2am | 2 | 1 | 5 | 5 | 2 | 1 |
| 2ap | 5 | 2 | 4 | 13 | 5 | 2 |
| 13a | 1 | 1 | 44 | 5 | 2 | 1 |
| 12a | 1 | 0.4 | 7 | 2 | 1 | 0.4 |
| 13 | 126 | 45 | 195 | 177 | 88 | 18 |
| 12 | 24 | 5 | 19 | 27 | 5 | 1 |
| 13b | 11 | 6 | 58 | 30 | 23 | 2 |
| 2aq | 7 | 4 | 12 | 12 | 39 | 5 |
| 10 | 57 | 51 | 33 | 59 | 149 | 15 |
| 14 | 4 | 2 | 3 | 5 | 22 | 1 |
| 5b | 46 | 10 | 45 | 36 | >1000 | 27 |
| 2ar | 29 | 4 | 14 | 14 | 258 | 13 |
| 2as | 3 | 1 | 2 | 1 | 51 | 2 |
| 2at | 38 | 4 | 30 | 21 | 264 | 23 |
| 2au | 5 | 1 | 2 | 1 | 40 | 2 |
| 2av | 26 | 5 | 11 | 9 | 236 | 8 |
| 5c | 1 | 0.8 | 6 | 2 | 2 | 0.5 |
| 5a | 28 | 20 | >1K | 350 | 73 | 12 |
| 5 | 10 | 9 | >1K | 218 | 258 | 7 |
| 2aw | 36 | 25 | >1K | 456 | 199 | 22 |
| 11 | 14 | 4 | >1K | 41 | 108 | 4 |
| 11a | 13 | 6 | >1K | 100 | 14 | 3 |
| 6 | 4 | 4 | 21 | 7 | 4 | 2 |
| 6a | 5 | 4 | 12 | 7 | 4 | 2 |
| 33 | 8 | 4 | 14 | 18 | 85 | 4 |
| 33a | 28 | 21 | 18 | 36 | 258 | 14 |
| 6b | 14 | 13 | 85 | 35 | 9 | 9 |
| 6c | 3 | 3 | 18 | 7 | 4 | 2 |
| 6d | 12 | 11 | 31 | 21 | 6 | 4 |
| 6g | 4 | 4 | 14 | 12 | 6 | 2 |
| 6f | 59 | 23 | >1K | 139 | 51 | 19 |
| 12b | 8 | 4 | 22 | 28 | 3 | 1 |
| 6e | 31 | 18 | 17 | 57 | 17 | 7 |
| 6h | 28 | 19 | 48 | 102 | 31 | 14 |
| 6i | 4 | 3 | 10 | 11 | 2 | 2 |

TABLE 23
In vitro potency (IC₅₀ values) of CPT free drugs (nmol/L) targeting
renal carcinoma cells (786-O), melanoma cells (A2058), pancreatic cancer cells (BxPC3),
non-small-cell lung cancer cells (Calu1), anaplastic large cell lymphoma cells (DEL,
DELBVR, Karpas299), Hodgkin's lymphoma cells (L540cy, Ls174T), breast cancer cells
(MDA-MB-231), acute myeloid leukemia cells (MOLM-13), and B-lymphocyte cancer cells
(SU-DHL4).

| | 786-O | A2058 | BxPC3 | Calu1 | DEL | DELBVR |
|---|---|---|---|---|---|---|
| Drug | IC₅₀ | IC₅₀ | IC₅₀ | IC₅₀ | IC₅₀ | IC₅₀ |
| 7w | 15 | ND | 13 | ND | 2 | 2 |
| 7n | 66 | ND | 47 | ND | 5 | 6 |
| 7i | 118 | ND | 57 | ND | 9 | 17 |
| 7h | 44 | ND | 43 | ND | 5 | 6 |
| 7aa | 131 | ND | 66 | ND | 17 | 19 |
| 7z | 123 | ND | 127 | ND | 17 | 16 |
| 8f | 267 | ND | 166 | ND | 14 | 30 |
| 8e | 104 | ND | 62 | ND | 7 | 10 |
| 8d | 15 | ND | 14 | ND | 2 | 2 |
| 8c | 27 | ND | 14 | ND | 3 | 4 |
| 8b | 102 | ND | 51 | ND | 8 | 13 |
| 8a | 11 | ND | 15 | ND | 1 | 2 |
| 8 | 113 | ND | 52 | ND | 8 | 16 |
| | Karpas299 | L540cy | Ls174T | MDAMB | MOLM- | SU-DHL- |
| Drug | IC₅₀ | IC₅₀ | IC₅₀ | 231 IC₅₀ | 13 IC₅₀ | 4 IC₅₀ |
| 7w | 8 | 6 | 16 | 26 | 26 | 6 |
| 7n | 57 | 23 | 39 | 96 | 137 | 21 |
| 7i | 84 | 48 | 110 | 183 | 183 | 32 |
| 7h | 57 | 23 | 40 | 67 | 93 | 21 |
| 7aa | 63 | 51 | 188 | 228 | 69 | 19 |
| 7z | 150 | 48 | 79 | 306 | 143 | 39 |
| 8f | 110 | 84 | 236 | 207 | 32 | 32 |
| 8e | 72 | 36 | 109 | 102 | 89 | 25 |
| 8d | 9 | 6 | 19 | 25 | 37 | 6 |
| 8c | 28 | 14 | 32 | 57 | 69 | 7 |
| 8b | 62 | 56 | 111 | 260 | 91 | 17 |
| 8a | 8 | 5 | 48 | 22 | 3 | 4 |
| 8 | 54 | 44 | 131 | 125 | 47 | 15 |

TABLE 24
In vitro potency (IC₅₀ values) of CPT ADCs (ng/ml) targeting renal
carcinoma cells (786-O), melanoma cells (A2058), pancreatic cancer cells (BxPC3), non-
small-cell lung cancer cells (Calu1), anaplastic large cell lymphoma cells (DEL, DELBVR,
Karpas299), Hodgkin's lymphoma cells (L540cy, Ls174T), breast cancer cells (MDA-MB-
231), acute myeloid leukemia cells (MOLM-13), and B-lymphocyte cancer cells (SU-DHL4).

| | 786-O | A2058 | BxPC3 | Calu1 | DEL | DELBVR |
|---|---|---|---|---|---|---|
| Drug | IC₅₀ | IC₅₀ | IC₅₀ | IC₅₀ | IC₅₀ | IC₅₀ |
| Ag4- | >1K | >1K | >1K | >1K | >1K | >1K |
| Ex_4a | | | | | | |
| Ag4- | >1K | >1K | >1K | >1K | 12 | 23 |
| Ex_4b | | | | | | |
| Ag4-Ex_4 | >1K | >1K | >1K | >1K | 1 | 4 |
| Ag4- | >1K | >1K | >1K | >1K | 176 | >1K |
| Ex_4c | | | | | | |
| Ag4-Ex_4f | >1K | >1K | >1K | >1K | 2 | 6 |
| Ag4- | >1000 | 637 | 845 | >1000 | 2 | 4 |
| Ex_14 | | | | | | |
| Ag4- | 880 | 541 | 609 | >1000 | 3 | 4 |
| Ex_14a | | | | | | |
| Ag1- | >1K | >1K | >1K | >1K | >1K | >1K |
| Ex_4a | | | | | | |
| Ag1- | >1K | >1K | 739 | >1K | 18 | 13 |
| Ex_4b | | | | | | |
| Ag1-Ex_4 | >1K | 241 | 106 | >1K | 3 | 3 |
| Ag1- | >1K | >1K | >1K | >1K | 139 | 55 |
| Ex_4c | | | | | | |
| Ag1-Ex_4f | >1K | >1K | 268 | >1K | 6 | 6 |
| Ag1- | 394 | 32 | 175 | >1000 | 4 | 3 |
| Ex_14 | | | | | | |
| Ag1- | 280 | 28 | 76 | >1000 | 4 | 4 |
| Ex_14a | | | | | | |
| | Karpas299 | L540cy | Ls174T | MDAMB | MOLM- | SU-DHL- |
| Drug | IC₅₀ | IC₅₀ | IC₅₀ | 231 IC₅₀ | 13 IC₅₀ | 4 IC₅₀ |
| Ag4- | ND | >1K | >1K | >1K | >1K | >1K |
| Ex_4a | | | | | | |
| Ag4- | 477 | 13 | >1K | >1K | >1K | >1K |
| Ex_4b | | | | | | |
| Ag4-Ex_4 | 535 | 7 | >1K | >1K | >1K | >1K |
| Ag4- | >1K | 54 | >1K | >1K | >1K | >1K |
| Ex_4c | | | | | | |
| Ag4-Ex_4f | 35 | 7 | >1K | >1K | >1K | >1K |
| Ag4- | 123 | 15 | ND | >1000 | >1000 | >1000 |
| Ex_14 | | | | | | |
| Ag4- | 18 | 9 | ND | >1000 | >1000 | >1000 |
| Ex_14a | | | | | | |
| Ag1- | ND | >1K | >1K | >1K | >1K | >1K |
| Ex_4a | | | | | | |
| Ag1- | 882 | 16 | >1K | >1K | 234 | 72 |
| Ex_4b | | | | | | |
| Ag1-Ex_4 | 6 | 79 | 35 | >1K | 60 | 9 |
| Ag1- | >1K | 61 | >1K | >1K | 432 | 521 |
| Ex_4c | | | | | | |
| Ag1-Ex_4f | 54 | 16 | 109 | >1K | 94 | 26 |
| Ag1- | 50 | 20 | ND | >1000 | 131 | 14 |
| Ex_14 | | | | | | |
| Ag1- | 16 | 16 | ND | >1000 | 180 | 23 |
| Ex_14a | | | | | | |

TABLE 25
In vitro potency (IC₅₀ values) of CPT free drugs (nmol/L) targeting
renal carcinoma cells (786-O), melanoma cells (A2058), pancreatic cancer cells (BxPC3),
non-small-cell lung cancer cells (Calu1), anaplastic large cell lymphoma cells (DEL,
DELBVR, Karpas299), Hodgkin's lymphoma cells (L540cy, Ls174T), breast cancer cells
(MDA-MB-231), acute myeloid leukemia cells (MOLM-13), and B-lymphocyte cancer cells
(SU-DHL4).

| Compound | 786-O | A2058 | BxPC3 | Calu1 | DEL | DELBVR |
|---|---|---|---|---|---|---|
| No. | IC₅₀ | IC₅₀ | IC₅₀ | IC₅₀ | IC₅₀ | IC₅₀ |
| 35 | 4 | 2 | 6 | 9 | 0.6 | 1 |
| 6j | 12 | 10 | 30 | 36 | 4 | 5 |
| 6l | 38 | 16 | 51 | 73 | 6 | 15 |
| 6k | 80 | 17 | 95 | 170 | 7 | 11 |
| 6p | ND | 2 | ND | 8 | 0.9 | 2 |
| 6o | ND | 4 | ND | 26 | 2 | 3 |
| 6n | ND | 16 | ND | 56 | 6 | 7 |
| 6m | ND | 7 | ND | 35 | 2 | 6 |
| 7ag | 0.9 | 0.6 | ND | 6 | 0.3 | 0.3 |
| 7ah | 0.8 | 0.5 | ND | 10 | 0.2 | 0.2 |
| 16 | 8 | 3 | ND | 67 | 1 | 1 |
| 15c | 22 | 6 | ND | 184 | 1 | 3 |
| 7aj | 99 | 57 | 103 | >1K | 14 | 16 |
| 7ai | 12 | 3 | 17 | 81 | 1 | 3 |
| | Karpas299 | L540cy | Ls174T | MDAMB | MOLM- | SU-DHL- |
| Drug | IC₅₀ | IC₅₀ | IC₅₀ | 231 IC₅₀ | 13 IC₅₀ | 4 IC₅₀ |
| 35 | 4 | 2 | 3 | 5 | 22 | 1 |
| 6j | 9 | 9 | 12 | 103 | 15 | 14 |
| 6l | 25 | 20 | 29 | 177 | 16 | 18 |
| 6k | 40 | 16 | 46 | 368 | 13 | 21 |
| 6p | 3 | 2 | 5 | 10 | 3 | 1 |
| 6o | 8 | 5 | 9 | 35 | 14 | 4 |
| 6n | 24 | 17 | 47 | 70 | 30 | 13 |
| 6m | 12 | 8 | 22 | 38 | 7 | 5 |
| 7ag | 1 | 0.9 | 3 | 9 | 2 | 0.8 |
| 7ah | 1 | 0.8 | 2 | 6 | 0.9 | 0.5 |
| 16 | 7 | 5 | 6 | 38 | 30 | 3 |
| 15c | 22 | 10 | >1K | 69 | 18 | 3 |
| 7aj | 55 | 56 | 64 | 706 | 81 | 34 |
| 7ai | 6 | 4 | 8 | 52 | 9 | 3 |

TABLE 26
In vitro potency (IC₅₀ values) of CPT ADCs (ng/ml) targeting renal
carcinoma cells (786-O), melanoma cells (A2058), pancreatic cancer cells (BxPC3), non-
small-cell lung cancer cells (Calu1), anaplastic large cell lymphoma cells (DEL, DELBVR,
Karpas299), Hodgkin's lymphoma cells (L540cy, Ls174T), breast cancer cells (MDA-MB-
231), acute myeloid leukemia cells (MOLM-13), and B-lymphocyte cancer cells (SU-DHL4).

| | 786-O | A2058 | BxPC3 | Calu1 | DEL | DELBVR |
|---|---|---|---|---|---|---|
| Drug | IC₅₀ | IC₅₀ | IC₅₀ | IC₅₀ | IC₅₀ | IC₅₀ |
| Ag1-Ex_4i | >1K | >1K | >1K | >1K | 4 | 15 |
| Ag4-Ex_4i | >1K | >1K | >1K | >1K | 63 | >1K |
| Ag1- | >1K | >1K | >1K | >1K | 6 | 6 |
| Ex_4g | | | | | | |
| Ag4- | 153 | 121 | 130 | 551 | 4 | 14 |
| Ex_4g | | | | | | |
| Ag1- | >1K | >1K | >1K | >1K | 65 | 52 |
| Ex_4k | | | | | | |
| Ag4- | >1K | >1K | >1K | >1K | 73 | >1K |
| Ex_4k | | | | | | |
| Ag1- | >1K | >1K | >1K | >1K | >1K | >1K |
| Ex_4h | | | | | | |
| Ag4- | >1K | >1K | >1K | >1K | >1K | >1K |
| Ex_4h | | | | | | |
| Ag1-Ex_4j | >1K | >1K | >1K | >1K | 474 | 233 |
| Ag4-Ex_4j | >1K | >1K | >1K | >1K | >1K | >1K |
| Ag4- | ND | >1K | ND | >1K | >1K | >1K |
| Ex_4ad | | | | | | |
| Ag1- | ND | >1K | ND | >1K | 55 | 53 |
| Ex_4ad | | | | | | |
| Ag4- | ND | >1K | ND | >1K | 5 | 16 |
| Ex_4ac | | | | | | |
| Ag1- | ND | >1K | ND | >1K | 10 | 14 |
| Ex_4ac | | | | | | |
| Ag4-Ex_4l | ND | >1K | ND | >1K | 68 | >1K |
| Ag1-Ex_4l | ND | >1K | ND | >1K | 119 | 65 |
| Ag4- | ND | >1K | ND | >1K | 9 | >1K |
| Ex 4m | | | | | | |
| Ag1- | ND | >1K | ND | >1K | 9 | 22 |
| Ex 4m | | | | | | |
| Ag4- | >1K | >1K | >1K | >1K | 0.3 | 0.8 |
| Ex 4q | | | | | | |
| Ag1- | 61 | 32 | >1K | >1K | 0.9 | 1 |
| Ex_4q | | | | | | |
| Ag1-Ex_4i | >1K | >1K | >1K | >1K | 4 | 15 |
| Ag4-Ex_4i | >1K | >1K | >1K | >1K | 63 | >1K |
| Ag1-Ex_4g | >1K | >1K | >1K | >1K | 6 | 6 |
| Ag4-Ex_4g | 153 | 121 | 130 | 551 | 4 | 14 |
| Ag1-Ex_4k | >1K | >1K | >1K | >1K | 65 | 52 |
| Ag4-Ex_4k | >1K | >1K | >1K | >1K | 73 | >1K |
| Ag1-Ex_4h | >1K | >1K | >1K | >1K | >1K | >1K |
| Ag4-Ex_4h | >1K | >1K | >1K | >1K | >1K | >1K |
| Ag1-Ex_4j | >1K | >1K | >1K | >1K | 474 | 233 |
| Ag4-Ex_4j | >1K | >1K | >1K | >1K | >1K | >1K |
| Ag4- | ND | >1K | ND | >1K | >1K | >1K |
| Ex 4ad | | | | | | |
| Ag1- | ND | >1K | ND | >1K | 55 | 53 |
| Ex 4ad | | | | | | |
| Ag4-Ex_4ac | ND | >1K | ND | >1K | 5 | 16 |
| Ag1-Ex_4ac | ND | >1K | ND | >1K | 10 | 14 |
| Ag4-Ex_4l | ND | >1K | ND | >1K | 68 | >1K |
| Ag1-Ex_4l | ND | >1K | ND | >1K | 119 | 65 |
| Ag4- | ND | >1K | ND | >1K | 9 | >1K |
| Ex_4m | | | | | | |
| Ag1- | ND | >1K | ND | >1K | 9 | 22 |
| Ex_4m | | | | | | |
| Ag4-Ex_4q | >1K | >1K | >1K | >1K | 0.3 | 0.8 |
| Ag1-Ex_4q | 61 | 32 | >1K | >1K | 0.9 | 1 |

TABLE 27
In vitro potency (IC₅₀ values) of CPT ADCs (ng/mL) targeting melanoma cells
(A2058), pancreatic cancer cells (BxPC3), anaplastic large cell lymphoma cells
(DEL, DELBVR, Karpas299), Hodgkin's lymphoma cells (L540cy, Ls174T), acute
myeloid leukemia cells (MOLM-13), and B-lymphocyte cancer cells (SU-DHL4).

| | A2058 | BxPC3 | DEL | DELBVR |
|---|---|---|---|---|
| Drug | IC₅₀ | IC₅₀ | IC₅₀ | IC₅₀ |
| Ag1-Ex_4f(8) | 344 | >1K | 7 | 4 |
| Ag4-Ex_4f(8) | >1K | >1K | 4 | 6 |
| Ag1-Ex_4(8) | 211 | 535 | 5 | 2 |
| Ag4-Ex_4(8) | >1K | >1K | 2 | 4 |
| Ag1-Ex_4x(8) | 223 | >1K | 3 | 4 |
| Ag4-Ex_4x(8) | >1K | >1K | 0.9 | 4 |
| Ag1-Ex_4y(8) | 539 | >1K | 2 | 3 |
| Ag4-Ex_4y(8) | >1K | >1K | 0.6 | 5 |
| Ag1-Ex_4ab(8) | 126 | >1K | 1 | 1 |
| Ag4-Ex_4ab(8) | >1K | >1K | 0.1 | 1 |
| Ag1-Ex_4aa(8) | 168 | 88 | 2 | 1 |
| Ag4-Ex_4aa(8) | >1K | >1K | 0.6 | 2 |
| Ag1-Ex_4z(8) | 342 | 233 | 2 | 3 |
| Ag4-Ex_4z(8) | >1K | >1K | 1 | 7 |

| | Karpas299 | Ls174T | MOLM-13 | SU-DHL-4 | L540cy |
|---|---|---|---|---|---|
| Drug | IC₅₀ | IC₅₀ | IC₅₀ | IC₅₀ | IC₅₀ |
| Ag1-Ex_4f(8) | 25 | ND | 160 | 22 | 14 |
| Ag4-Ex_4f(8) | 19 | ND | >1K | >1K | 8 |
| Ag1-Ex_4(8) | 123 | ND | 131 | 16 | 9 |
| Ag4-Ex_4(8) | 59 | ND | >1K | >1K | 6 |
| Ag1-Ex_4x(8) | 51 | 56 | 106 | 14 | ND |
| Ag4-Ex_4x(8) | 7 | >1K | >1K | >1K | ND |
| Ag1-Ex_4y(8) | 27 | 18 | 39 | 11 | ND |
| Ag4-Ex_4y(8) | 16 | >1K | >1K | >1K | ND |
| Ag1-Ex_4ab(8) | 46 | >1K | 23 | 9 | ND |
| Ag4-Ex_4ab(8) | 44 | >1K | >1K | >1K | ND |
| Ag1-Ex_4aa(8) | 21 | 17 | 34 | 7 | ND |
| Ag4-Ex_4aa(8) | 16 | >1K | >1K | >1K | ND |
| Ag1-Ex_4z(8) | 63 | 72 | 28 | 7 | ND |
| Ag4-Ex_4z(8) | 33 | >1K | >1K | >1K | ND |

In vivo Model Methods

All experiments were conducted in concordance with the Animal Care and Use Committee in a facility fully accredited by the Association for Assessment and Accreditation of Laboratory Animal Care. Efficacy experiments were conducted in the L540cy, OV90, EBC-1, and 768-0 xenografts models. Tumor cells, as a cell suspension, were implanted sub cutaneous in immune-compromised SCID or nude mice. Upon tumor engraftment, mice were randomized to study groups (5 mice per group) when the average tumor volume reached about 100 mm³. The ADC or controls were dosed once via intraperitoneal injection. The average number of drug-linker attached to an antibody is indicated in the parenthesis next to the ADC (also referred to herein as Drug-Antibody Ratio (DAR) number, e.g., DAR4, DAR8, etc.). Ag1 refers to an antibody that targets a ubiquitously expressed cell surface antigen. Ag2 refers to an antibody that targets a surface antigen expressed on tumor cells and is involved in self-tolerance. Ag3 refers to an antibody that targets 0-glycans overexpressed on the surface of cancer cells. Ag4 refers to an antibody that targets a surface antigen characteristically overexpressed in hematopoietic malignancies. Ag5 refers to an antibody that targets a surface antigen highly expressed in hemtologic malignancies and renal cell carcinoma. h00 is a non-binding control antibody. Tumor volume as a function of time was determined using the formula (L×W2)/2. Animals were euthanized when tumor volumes reached 750 mm³. Mice showing durable regressions were terminated after 10-12 weeks post implant.

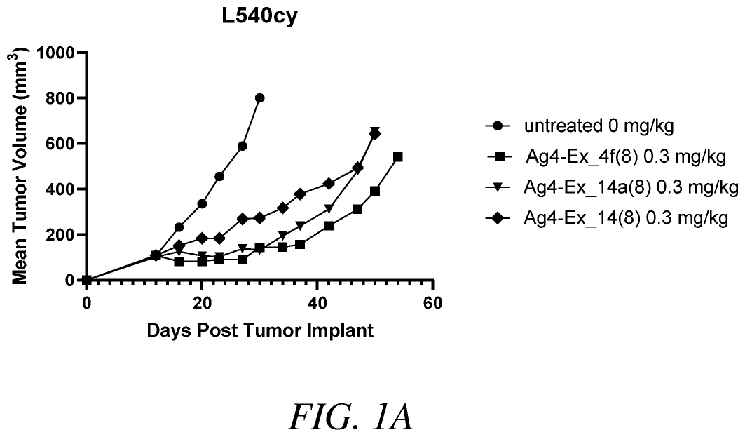

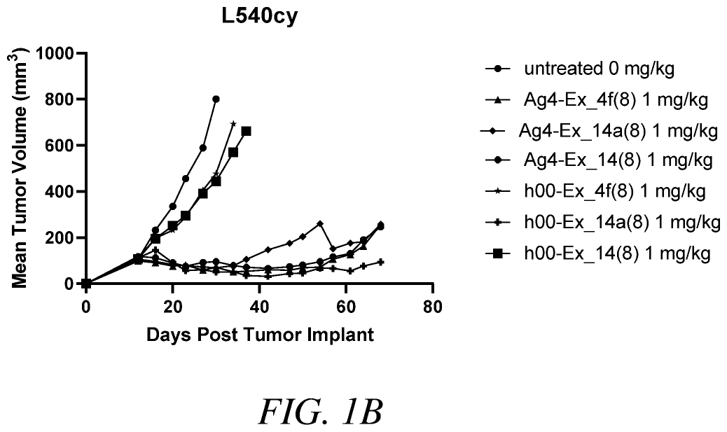

Animals were implanted with L540cy cells. After 12 days, the animals were sorted into groups with an average tumor size of 100 mm³, and then treated with a single dose of camptothecin ADC Ag4-Ex_4f(8), Ag4-Ex_14a(8) or Ag4-Ex_14(8) at 0.3 or Ag4-Ex_4f(8), Ag4-Ex_14a(8), Ag4-Ex-14(8), h00-Ex_4f(8), h00-Ex_14a(8) or h00-Ex_14a(8) at 1 mg/kg. Animals were evaluated for tumor size and in-life signs during the course of the study. The results are shown in FIGS. 1A and 1B.

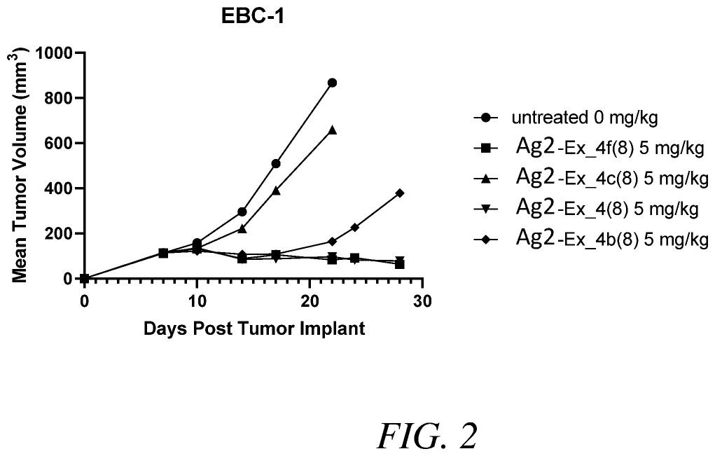

Animals were implanted with EBC-1 cells. On day 7, the animals were sorted into groups with an average tumor size of 100 mm³, and then treated with a single dose of camptothecin ADC Ag2-Ex_4f(8), Ag2-Ex_4c(8), Ag2-Ex_4b(8) or Ag2-Ex_4(8), at 5 mg/kg. Animals were evaluated for tumor size and in-life signs during the course of the study. The results are shown in FIG. 2.

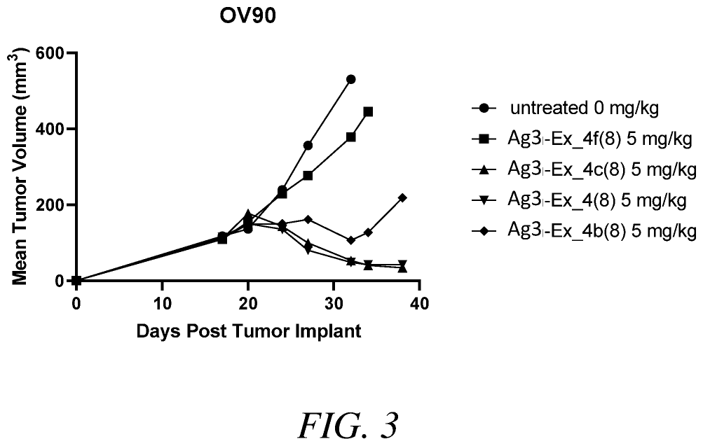

Animals were implanted with OV-90 cells. After 17 days, the animals were sorted into groups with an average tumor size of 100 mm³, and then treated with a single dose of camptothecin ADC Ag3-Ex_4f(8), Ag3-Ex_4c(8), Ag3-Ex_4b(8) or Ag3-Ex_4(8), at 5 mg/kg. Animals were evaluated for tumor size and in-life signs during the course of the study. The results are shown in FIG. 3.

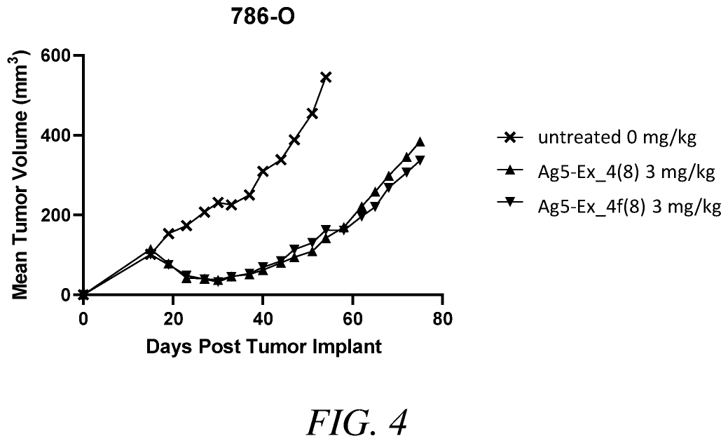

Animals were implanted with 786-0 cells. After 15 days, the animals were sorted into groups with an average tumor size of 100 mm³, and then treated with a single dose of camptothecin ADC Ag5-Ex_4(8) or Ag5-Ex_4f(8), at 3 mg/kg. Animals were evaluated for tumor size and in-life signs during the course of the study. The results are shown in FIG. 4.

### ENUMERATED EMBODIMENTS

Embodiment 1. A Camptothecin Conjugate having the formula of

L-(Q-D)p

or a salt thereof, wherein
L is a Ligand Unit from a targeting agent, in particular from an antibody that selectively binds to a cancer cell antigen;
subscript p is an integer ranging from 1 to 16;
Q is a Linker Unit having a formula selected from the group consisting of:
   Z-A-, -Z-A-RL-, -Z-A-RL-Y-, -Z-A-S*-RL-, -Z-A-S*-RL-Y-, -Z-A-S*-W-, -Z-A-S*-W-RL-, -Z-A-B(S*)-RL-, -Z-A-B(S*)-W-, -Z-A-B(S*)-W-RL- and -Z-A-B(S*)-RL-Y-,
wherein Z is a Stretcher Unit;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit D having a formula of

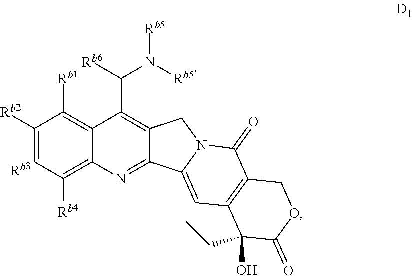

or a salt thereof; wherein
R^{b1} is selected from the group consisting of H, halogen, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
   each R^{b5} and R^{b5}′ are independently selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃ C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a} are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

Embodiment 2. The Camptothecin Conjugate of embodiment 1, wherein D has a formula selected from the group consisting of

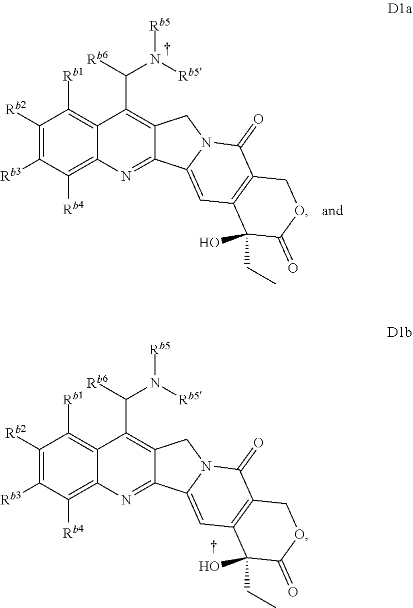

or a salt thereof, wherein the dagger indicates the site of covalent attachment of D to the secondary linker of the drug linker moiety.

Embodiment 3. The Camptothecin Conjugate of embodiment 2, wherein D has a formula selected from selected from the group consisting of

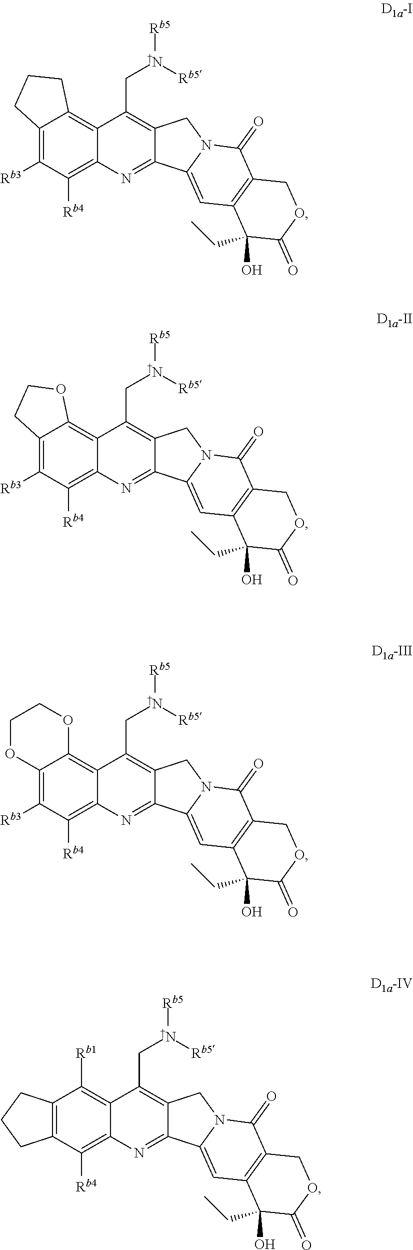

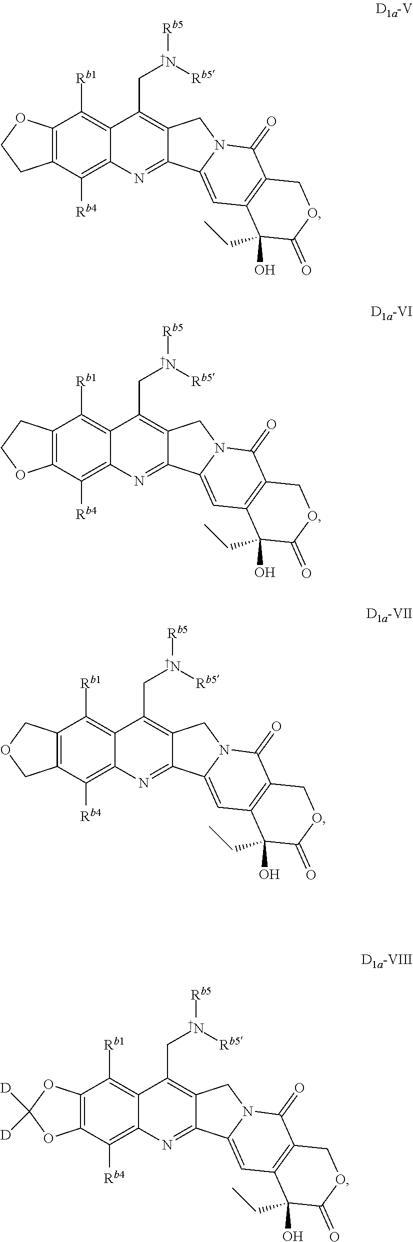

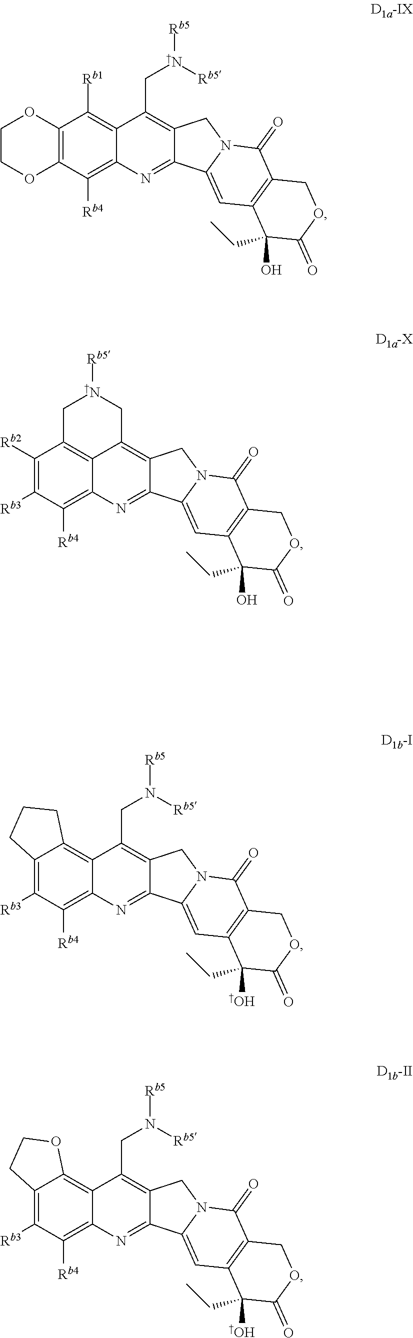

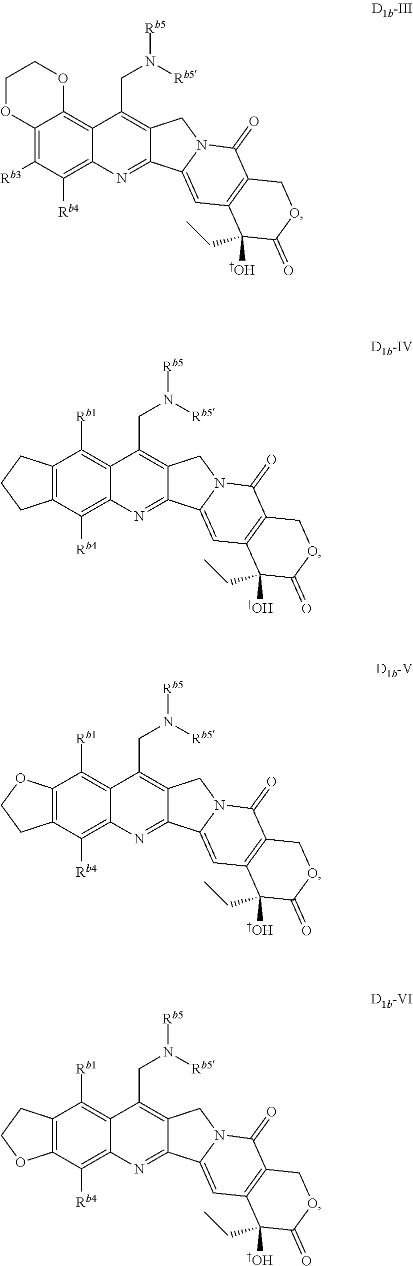

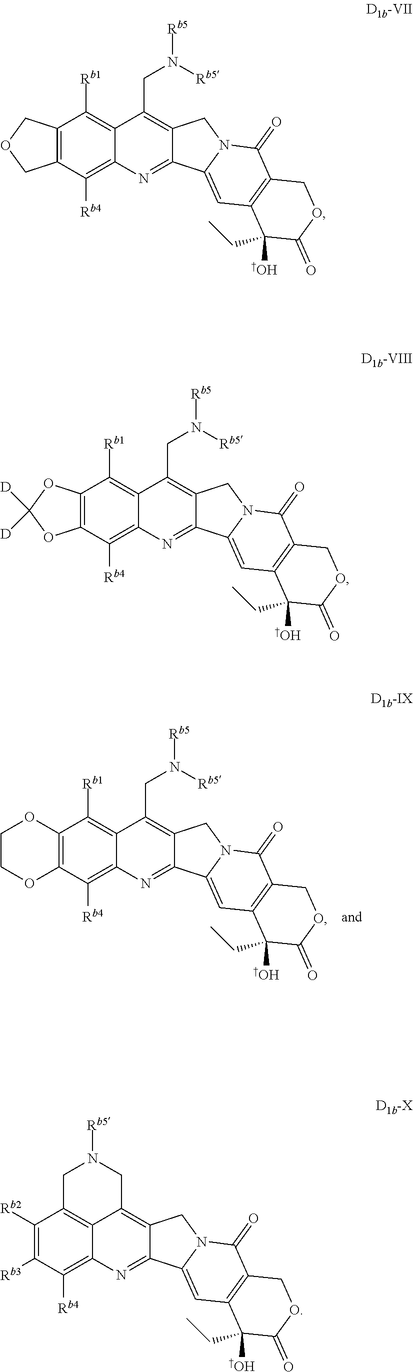

Embodiment 4. The Camptothecin Conjugate of embodiment 2, wherein D has a formula selected from the group consisting of

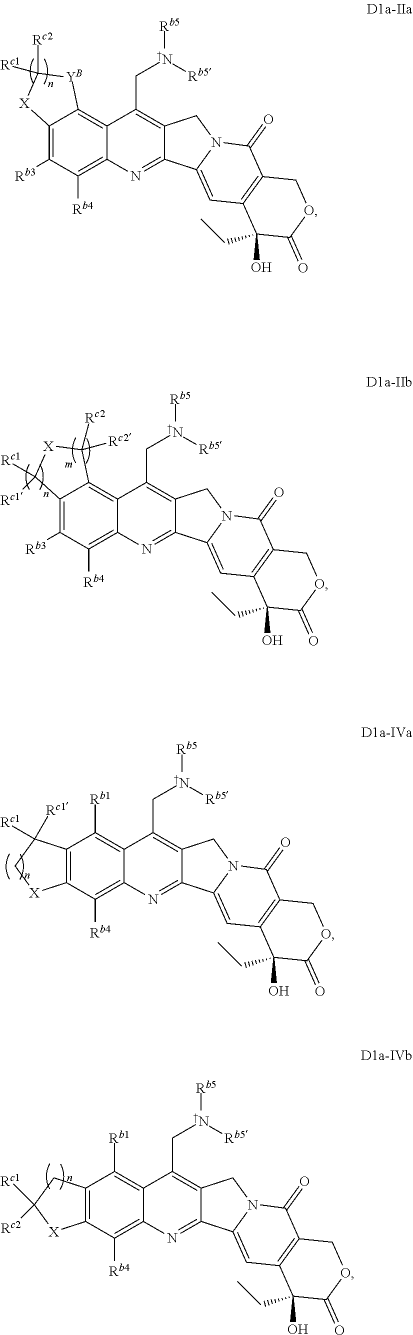

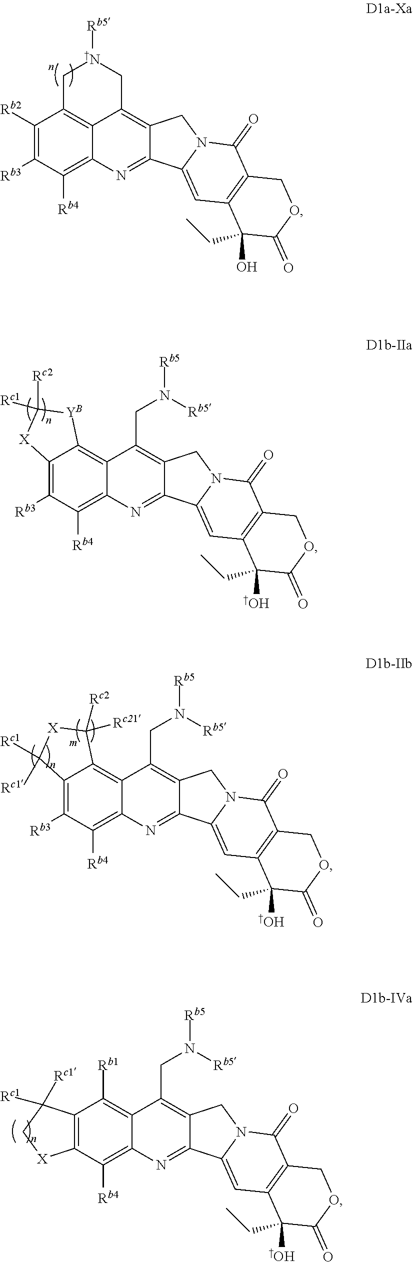

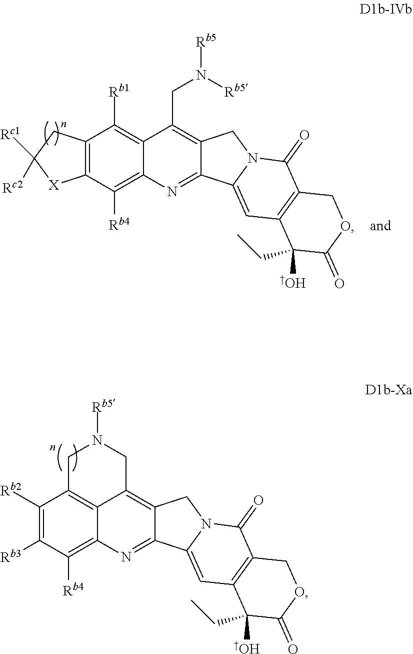

wherein
X and Y^{B} are each independently 0, S, S(O)₂, CR^{x}′, or NR^{x};
R^{x} and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently

(i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}, and —SR^{a}, C₁-C₆ alkyl-C(O)—, C₁-C₆ alkyl-NR^{a}—C(O)—, and C₁-C₆ alkyl-S(O)₂—; or

(ii) taken together with R^{b1} and the intervening atoms to form a 5- to 7-membered carbocyclo or heterocyclo; or

(iii) taken together with R^{x}′ and the intervening atoms to form a 3 to 6-membered carbocyclo or heterocyclo; and when m and n are both present, the sum of m+n is 2 or 3.

Embodiment 5. The Camptothecin Conjugate of embodiment 2, wherein D has a formula selected from selected from the group consisting of

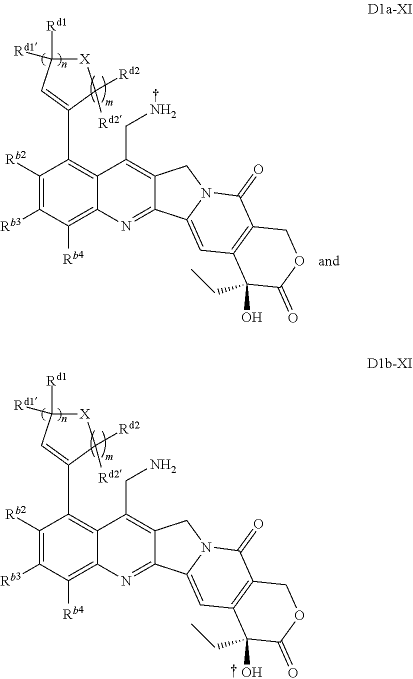

wherein
R^{d1}, R^{d1}′, R^{d2}, and R^{d2}′ are each independently selected from the group consisting of H, halogen, C₁-C₆ alkyl, C₁-C₆ haloalkyl, —OR^{a}, —NR^{a}R^{a}, and —SR^{a}, C₁-C₆ alkyl-C(O)—, C₁-C₆ alkyl-NR^{a}—C(O)—, and C₁-C₆ alkyl-S(O)₂—.

Embodiment 6. The Camptothecin Conjugate of embodiment 2, wherein D has a formula selected from the group consisting of

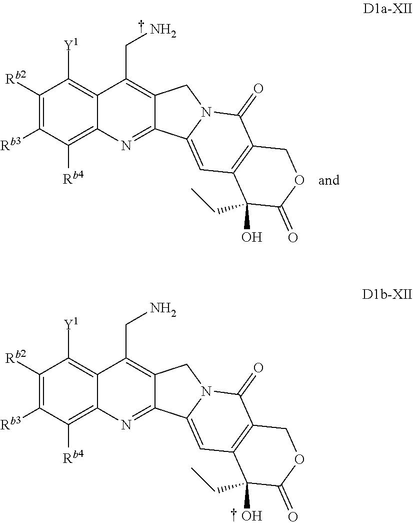

wherein

Y¹ is a 5- or 6-membered heteroaryl, optionally substituted with halogen, C₁-C₆ alkyl, C₁-C₆ haloalkyl, C₁-C₆ hydroxyalkyl, C₁-C₆ aminoalkyl, or C₁-C₆ alkyl-S(O)₂,

Embodiment 7. The Camptothecin Conjugate of embodiment 2, wherein D has a formula selected from the group consisting of

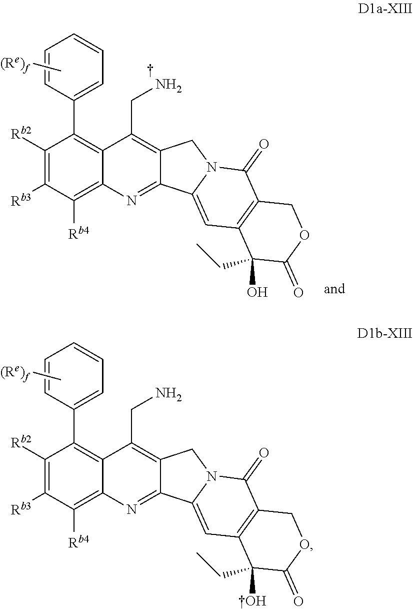

wherein
each R is independently selected from the group consisting of halogen, —OH, —NH₂, C₁-C₆ alkyl, C₁-C₆ haloalkyl, C₁-C₆ hydroxyalkyl, C₁-C₆ alkyl-S(O)₂—, and C₁-C₆ alkyl-NR^{a}-C(O)—; and f is 0, 1, 2, 3, 4, or 5.

Embodiment 8. The Camptothecin Conjugate of embodiment 2, wherein D has a formula selected from the group consisting of

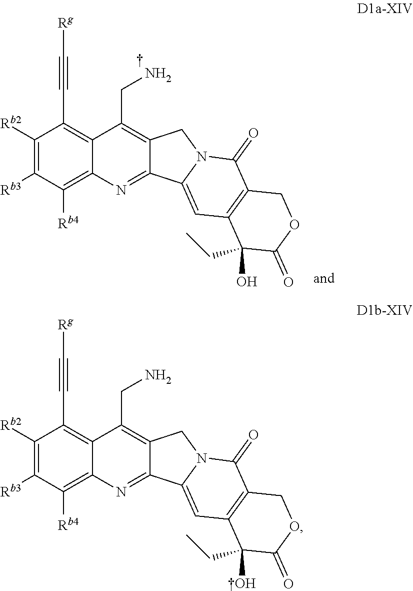

wherein

R^{g} is H, C₁-C₆ alkyl, or 3 to 8-membered heterocyclyl.

Embodiment 9. The Camptothecin Conjugate of embodiment 2, wherein D has a formula selected from the group consisting of

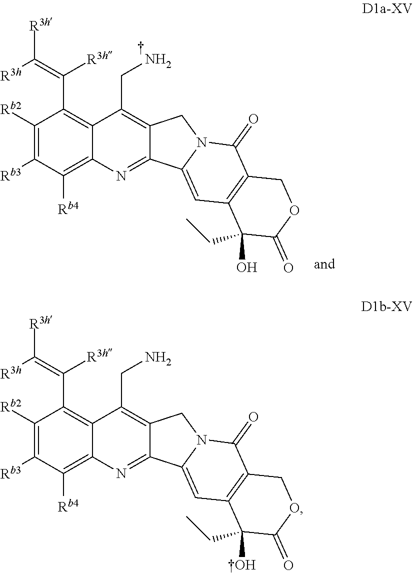

wherein
R^{3b}, R^{3b}′, and R^{3b}″ are each independently selected from the group consisting of H, C₁-C₆ alkyl, C₁-C₆ hydroxyalkyl, C₁-C₆ aminoalkyl, —C(O)—C₁-C₆ alkyl, —C(O)O-C₁-C₆ alkyl, —C(O)NH—C₁-C₆ alkyl, C₆-C₁₀ aryl, —C₆-C₁₀ aryl-C₁-C₆ alkyl, and -C₆-C₁₀ aryl-C₁-C₆ alkoxy; each optionally substituted with, C₁-C₆ alkyl, C₁-C₆ haloalkyl, —OR^{a}, —NR^{a}R^{a}, and —SR^{a}.

Embodiment 10. The Camptothecin Conjugate of embodiment 2, wherein D has a formula selected from the group consisting of

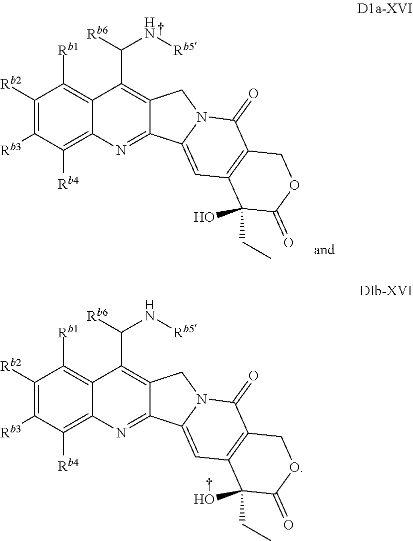

Embodiment 11. The Camptothecin Conjugate of any one of embodiments 1-10, wherein Q is a Linker Unit having the formula selected from the group consisting of:
Z-A-RL-; -Z-A-RL-Y-; -Z-A-S*-RL-; -Z-A-B(S*)-RL-; -Z-A-S* -RL-Y-; and -Z-A-B(S*)-RL-Y-,
wherein A is a Connector Unit and RL is a Glycoside (e.g., Glucuronide) Unit.

Embodiment 12. The Camptothecin Conjugate of embodiment 11, wherein the Glycoside (e.g., Glucuronide) Unit has the formula of:

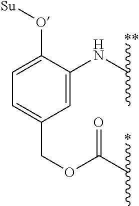

wherein
Su is a hexose form of a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q,
in particular the Glycoside (e.g., Glucuronide) Unit has the formula of:

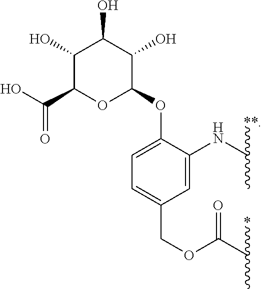

Embodiment 13. The Camptothecin Conjugate of embodiment 11, wherein
Q is a Linker Unit having the formula of -Z-A-RL-Y-, -Z-A -S*-RL-Y- or -Z-A-B(S*)-RL-Y-; and
Spacer Unit (Y) has the formula of:

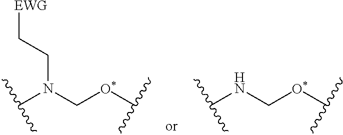

wherein EWG is an electron-withdrawing group;
O* represent the oxygen atom from a hydroxy substituent of D;
the wavy line adjacent to the nitrogen atom indicates the site of covalent attachment to the carbonyl carbon atom of the Glycoside (e.g., Glucuronide) Unit; and
the wavy line adjacent to O* indicates the site of covalent attachment to the remainder of D, or
the Spacer Unit (Y) has the formula of:

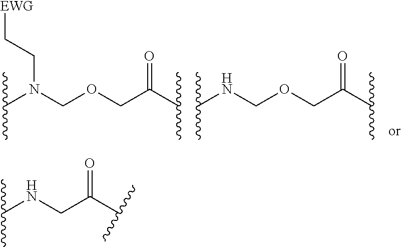

wherein
EWG is an electron-withdrawing group;
the wavy line adjacent to the nitrogen atom indicates the site of covalent attachment to the carbonyl carbon atom of the Glycoside (e.g., Glucuronide) Unit; and
the wavy line adjacent to the carbonyl carbon atom indicates the site of covalent attachment to the nitrogen atom of the amino substituent of D.

Embodiment 14. The Camptothecin Conjugate of any one of embodiments 1-10, wherein

Q is a Linker Unit having a formula selected from the group consisting of:
Z-A-; -Z-A-S*-W- and -Z-A-B(S*)-W-,
wherein A is a Connector Unit, or
Q is a Linker Unit having a formula selected from the group consisting of:
   Z-A-RL-, -Z-A-S*-RL-; -Z-A-B(S*)-RL-, -Z-A-S*-W-RL-, and -Z-A-B(S*)-W-RL-,
wherein A is a Connector Unit and RL is a Releasable linker other than a Glycoside (e.g., Glucuronide) Unit.

Embodiment 15. The Camptothecin Conjugate of embodiment 14, wherein
Q is a Linker Unit having the formula selected from the group consisting of -Z-A-RL-, -Z-A-S*-RL- and -Z-A-S*-W-RL-, wherein
RL has the formula:

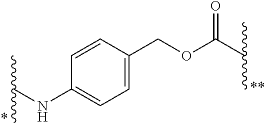 [SMILES: *Nc1ccc(COC(*)=O)cc1]

wherein
the wavy line marked with a double asterisk (**) indicates the site of covalent attachment to D; and
the wavy line marked with a single asterisk (*) indicates the point of covalent attachment to A, S* or W.

Embodiment 16. The Camptothecin Conjugate of embodiment 14 or 15, wherein
-Q-D has the formula of -Z-A-S*-W-RL-D, wherein
D is covalently attached to Q via the nitrogen atom of an amine functional group of D; and
W is an Amino Acid Unit selected from the group consisting of N-methyl-glycine (sarcosine), N-methyl-alanine, N-methyl-β-alanine, valine, N-methyl-valine, or
D is covalently attached to Q via an oxygen atom of the hydroxyl substituent on the lactone ring of D; and
W is an Amino Acid Unit selected from the group consisting glutamic acid or lysine.

Embodiment 17. The Camptothecin Conjugate of embodiment 16, wherein -Z-A-comprises a succinimido-alkanoyl moiety or succinimido and triazole moieties, each optionally having the succinimide ring in hydrolyzed form as a succinic acid amide moiety, or a succinic acid amide moiety derivable from mDPR of a Camptothecin-Linker Compound, or
wherein -Z-A- has the formula of:

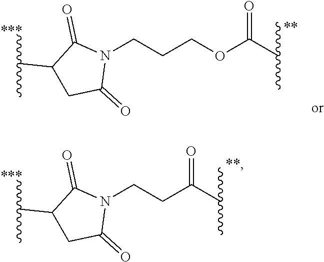

optionally having the succinimide ring in hydrolyzed form as a succinic acid amide moiety, wherein the wavy line marked with a double asterisk (**) indicates the site of covalent attachment to S*; and the wavy line marked with a triple asterisk (***) indicates the point of covalent attachment to a sulfur atom of L.

Embodiment 18. The Camptothecin Conjugate of embodiment 14, wherein
Q is a Linker Unit having the formula selected from the group consisting of -Z-A-S*-RL- and -Z-A-S*-W-RL-, wherein
S* has the formula of:

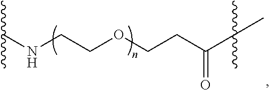

wherein subscript n is an integer ranging from 2 to 36,
the wavy line adjacent to the nitrogen atom indicates the site of covalent attachment to a carbonyl carbon atom of A, and the wavy adjacent to the carbonyl carbon atom indicates the site of covalent attachment to the nitrogen atom of the amine functional group of RL of -Z-A-S*-RL- or W of -Z-A-S*-W-RL-,
in particular, -Z A- in either formula of Q has the formula of:

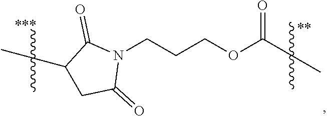

wherein the wavy line marked with a double asterisk (**) indicates the site of covalent attachment to the nitrogen atom of the amine functional group of S*; and the wavy line marked with a triple asterisk (***) indicates the point of covalent attachment to a sulfur atom of L.

Embodiment 19. The Camptothecin Conjugate of embodiment 14, wherein Q is a Linker Unit of formula -Z-A-S*-W- or -Z-A-S—W-RL-, wherein -Z-A-S*-W- in either formula has the formula of:

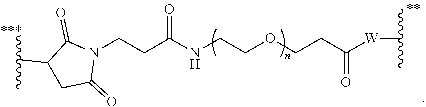

optionally having the succinimide ring in hydrolyzed form as a succinic acid amide moiety, wherein subscript n is an integer ranging from 2 to 10, preferably ranging from 2 to 4; the wavy line marked with a double asterisk (**) indicates the site of covalent attachment to D or RL; and the wavy line marked with a triple asterisk (***) indicates the point of covalent attachment to a sulfur atom of L.

Embodiment 20. A Camptothecin-Linker compound having a formula selected from the group consisting of:
(i) Z′-A-RL-D;
(ii) Z′-A-RL-Y-D;
(iii) Z′-A-S*-RL-D;
(iv) Z′-A-S*-RL-Y-D;
(v) Z′-A-B(S*)-RL-D;
(vi) Z′-A-B(S*)-RL-Y-D;
(vii) Z′-A-D
(viii) Z′-A-S*-W-D
(ix) Z′-A-B(S*)-W-D
(x) Z′-A-S*-W-RL-D; and
(xi) Z′-A-B(S*)-W-RL-D
   wherein
Z′ is a Stretcher Unit precursor;
A is a bond or a Connector Unit;
B is a Parallel Connector Unit;
S* is a Partitioning Agent;
RL is a Releasable Linker;
Y is a Spacer Unit; and
D is a Drug Unit D having a formula of

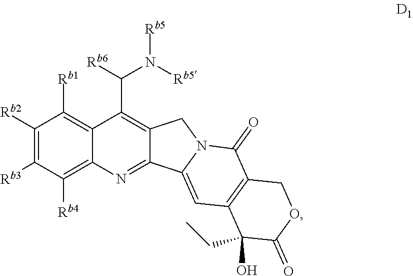

or a salt thereof; wherein
R^{b1} is selected from the group consisting of H, halogen, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b1} is combined with R^{b2}, R^{b5}, or R^{b6} and the intervening atoms to form a 5-, 6-, or 7-membered carbocyclo or heterocyclo; R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3-to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
   each R^{b5} and R^{b5}′ are independently selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a} are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

Embodiment 21. The Camptothecin-Linker compound of embodiment 20, wherein D has a formula selected from the group consisting of

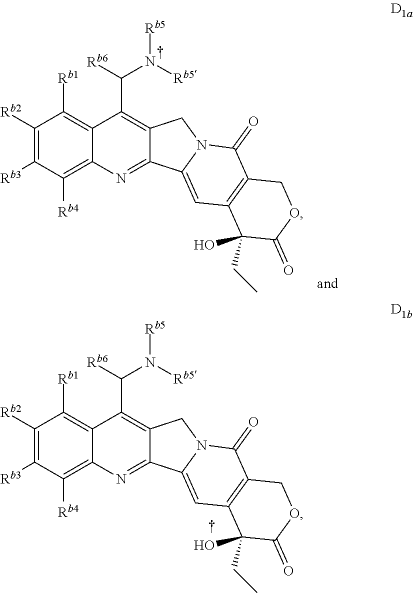

or a salt thereof, wherein the dagger indicates the site of covalent attachment of D to the secondary linker of the drug linker moiety.

Embodiment 22. The Camptothecin-Linker compound of embodiment 20, wherein D has a formula selected from selected from the group consisting of

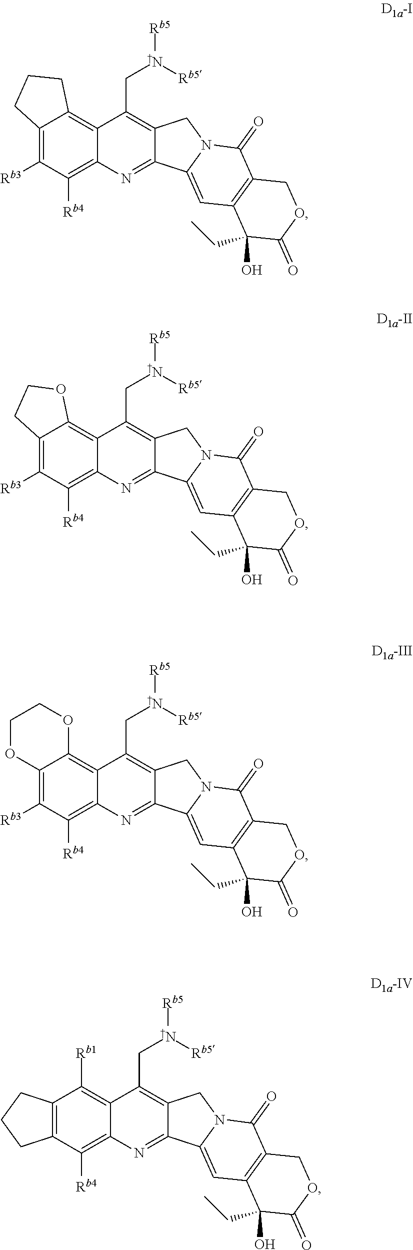

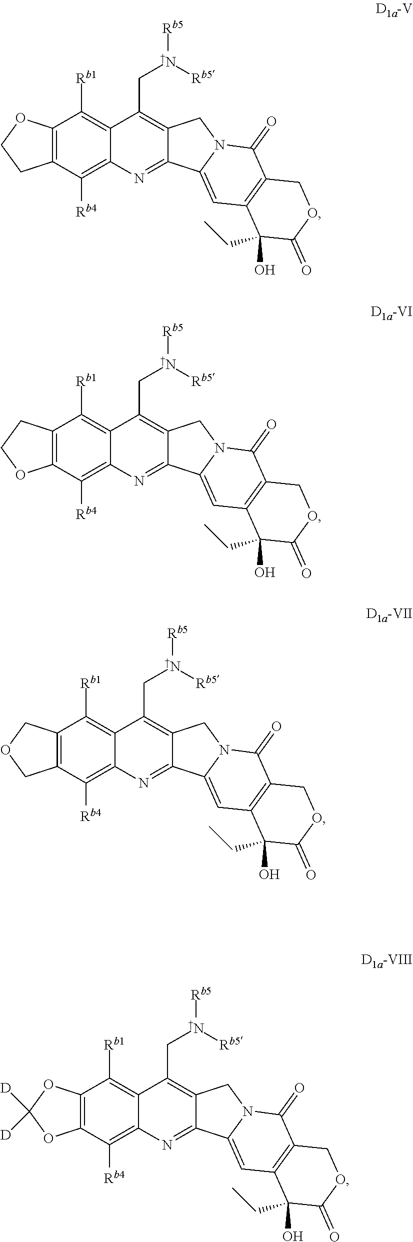

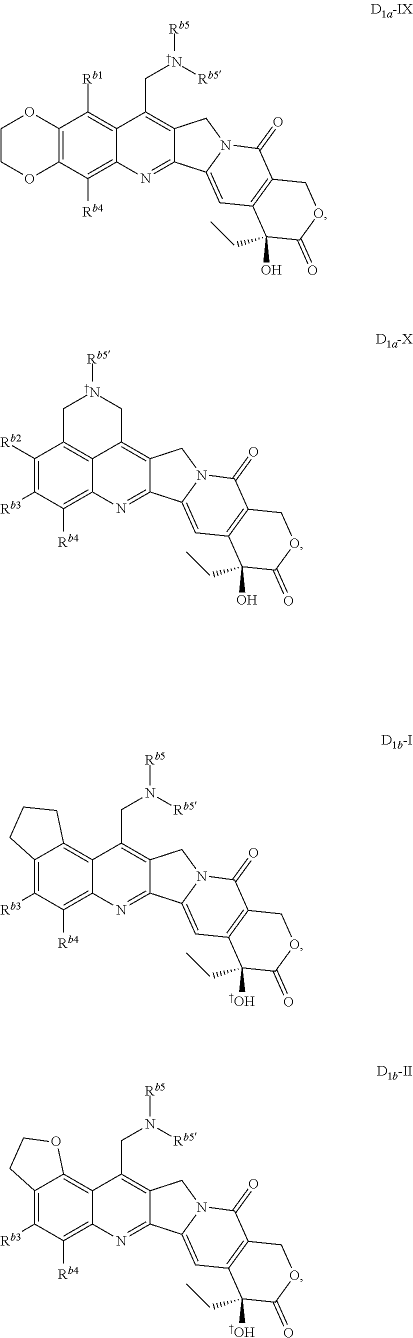

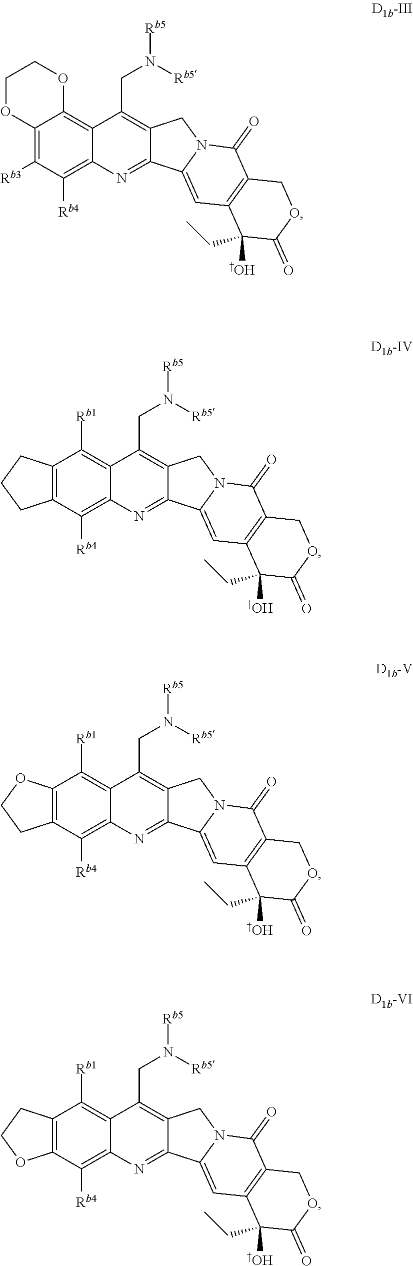

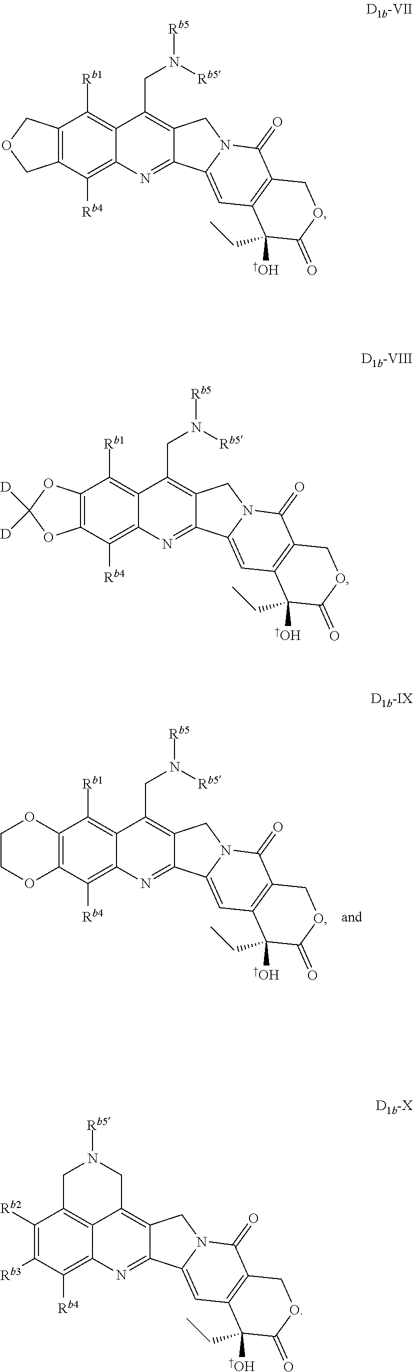

Embodiment 23. The Camptothecin-Linker compound of embodiment 20, wherein D has a formula selected from the group consisting of

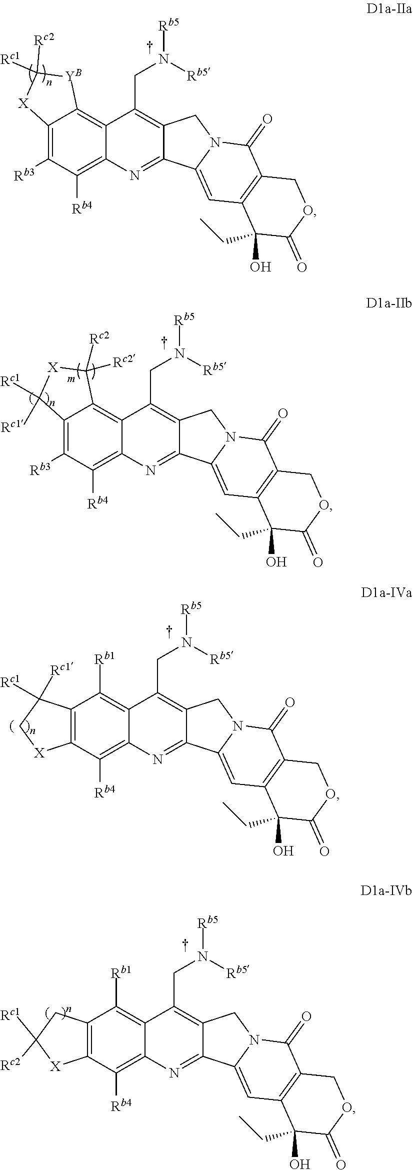

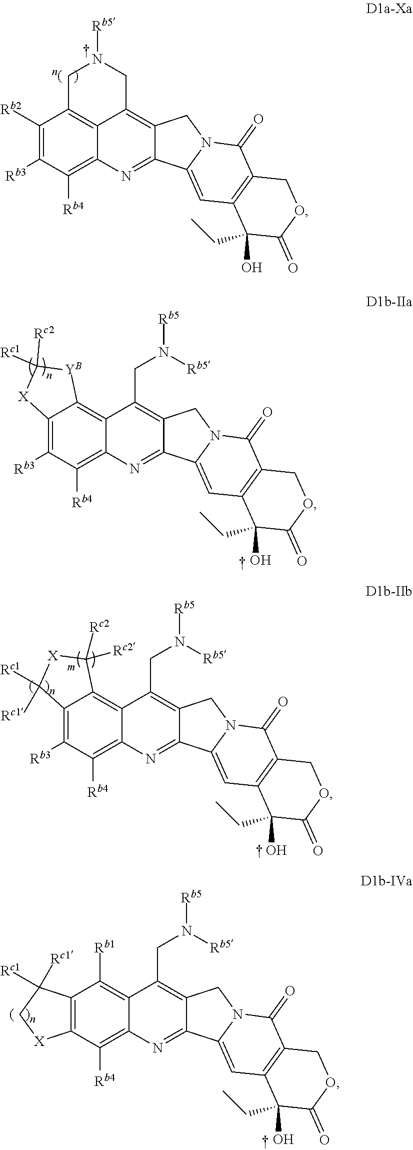

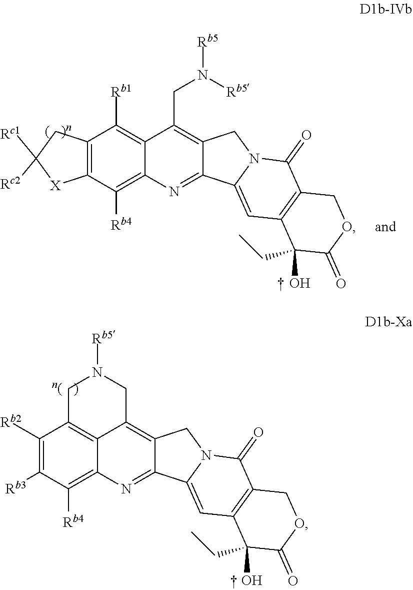

wherein
X and Y^{B} are each independently 0, S, S(O)₂, CR^{x}R^{x}′, or NR^{x};
R^{x} and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently

(i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}′, and —SR^{a}, C₁-C₆ alkyl-C(O)—, C₁-C₆ alkyl-NR^{a}—C(O)—, and C₁-C₆ alkyl-S(O)₂—; or

(ii) taken together with R^{b1} and the intervening atoms to form a 5- to 7-membered carbocyclo or heterocyclo; or

(iii) taken together with R^{x}′ and the intervening atoms to form a 3 to 6-membered carbocyclo or heterocyclo; and
when m and n are both present, the sum of m+n is 2 or 3.

Embodiment 24. The Camptothecin-Linker compound of embodiment 20, wherein D has a formula selected from selected from the group consisting of

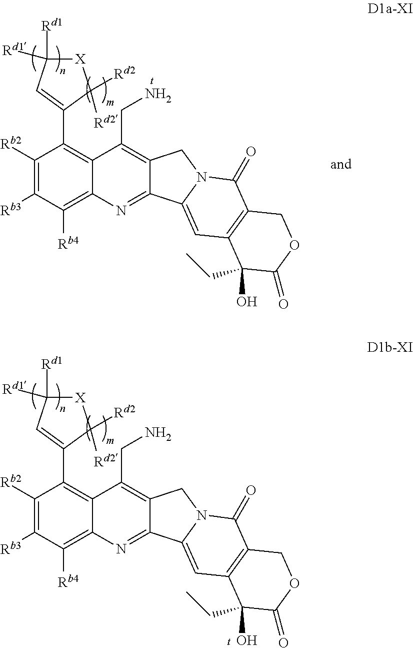

wherein
R^{d1}, R^{d1}′, R^{d2}, and R^{d2}′ are each independently selected from the group consisting of H, halogen, C₁-C₆ alkyl, C₁-C₆ haloalkyl, —OR^{a}, —NR^{a}R^{a}, and —SR^{a}, C₁-C₆ alkyl-C(O)—, C₁-C₆ alkyl-NR^{a}—C(O)—, and C₁-C₆ alkyl-S(O)₂—.

Embodiment 25. The Camptothecin-Linker compound of embodiment 20, wherein D has a formula selected from the group consisting of

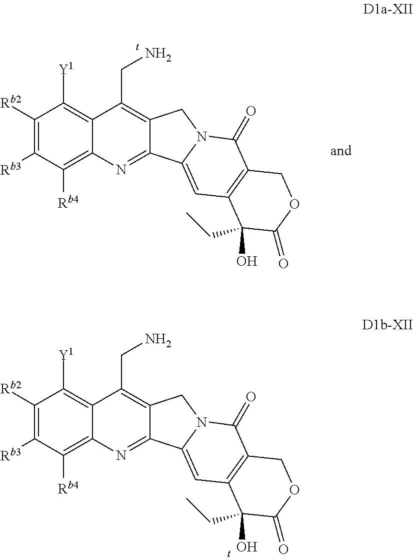

wherein
Y¹ is a 5- or 6-membered heteroaryl, optionally substituted with halogen, C₁-C₆ alkyl, C₁-C₆ haloalkyl, C₁-C₆ hydroxyalkyl, C₁-C₆ aminoalkyl, or C₁-C₆ alkyl-S(O)₂,

Embodiment 26. The Camptothecin-Linker compound of embodiment 20, wherein D has a formula selected from the group consisting of

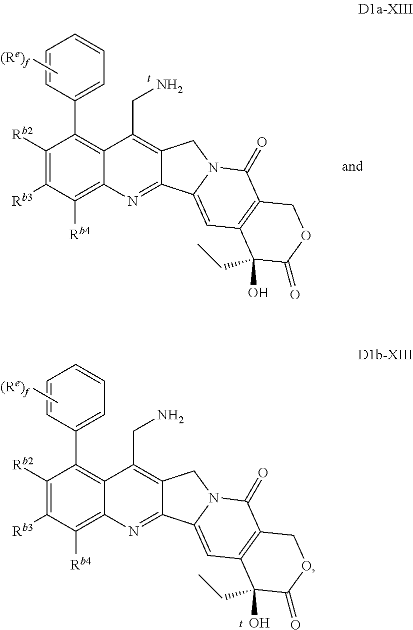

wherein
each R is independently selected from the group consisting of halogen, —OH, —NH₂, C₁-C₆ alkyl, C₁-C₆ haloalkyl, C₁-C₆ hydroxyalkyl, C₁-C₆ alkyl-S(O)₂—, and C₁-C₆ alkyl-NR^{a}-C(O)—; and f is 0, 1, 2, 3, 4, or 5.

Embodiment 27. The Camptothecin-Linker compound of embodiment 20, wherein D has a formula selected from the group consisting of

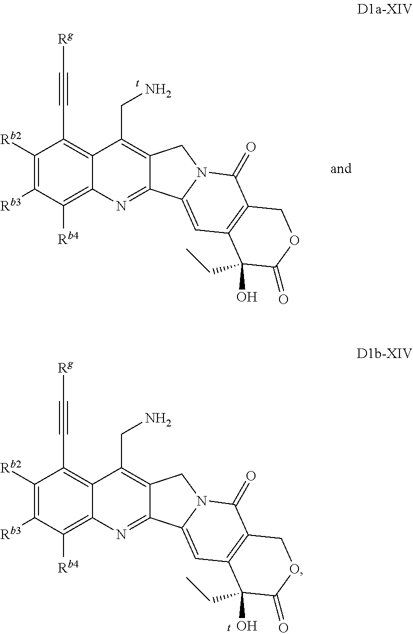

wherein
R^{g} is H, C₁-C₆ alkyl, or 3 to 8-membered heterocyclyl.

Embodiment 28. The Camptothecin-Linker compound of embodiment 20, wherein D has a formula selected from the group consisting of

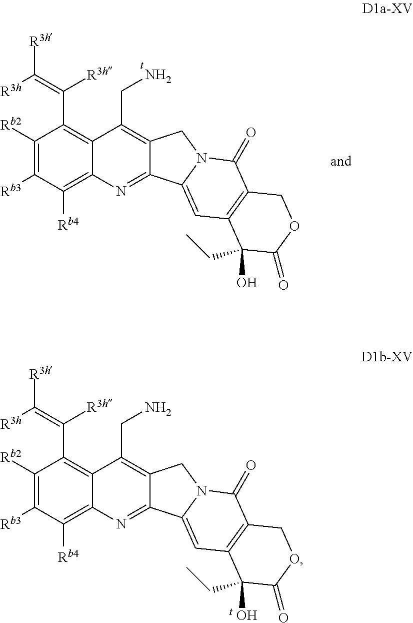

wherein
R^{3b}, R^{3b}′, and R^{3b}″ are each independently selected from the group consisting of H, C₁-C₆ alkyl, C₁-C₆ hydroxyalkyl, C₁-C₆ aminoalkyl, —C(O)—C₁-C₆ alkyl, —C(O)O-C₁-C₆ alkyl, —C(O)NH—C₁-C₆ alkyl, C₆-C₁₀ aryl, —C₆-C₁₀ aryl-C₁-C₆ alkyl, and -C₆-C₁₀ aryl-C₁-C₆ alkoxy; each optionally substituted with, C₁-C₆ alkyl, C₁-C₆ haloalkyl, —OR^{a}, —NR^{a}R^{a}, and —SR^{a}.

Embodiment 29. The Camptothecin-Linker compound of embodiment 20, wherein D has a formula selected from the group consisting of

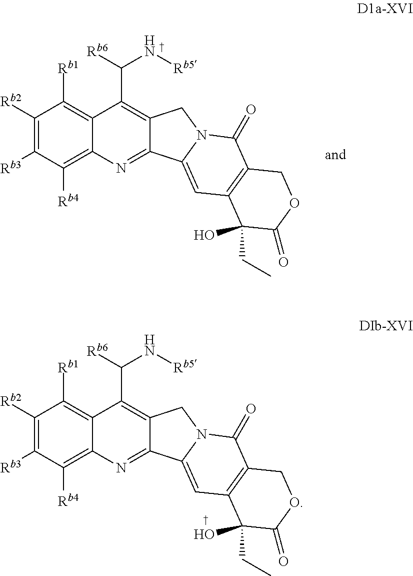

Embodiment 30. The Camptothecin-Linker compound of any one of embodiments 20 29 having the formula selected from the group consisting of formula (i), formula (ii); formula (iii), formula (iv), formula (v) and formula (vi), wherein A is a Connector Unit; and RL is a Glycoside (e.g., Glucuronide) Unit, in particular, having the structure of:

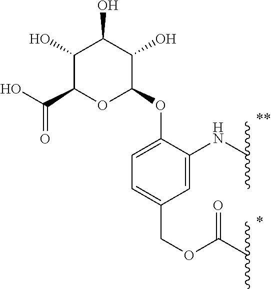 [SMILES: *Nc1cc(COC(*)=O)ccc1O[C@H]1O[C@@H](C(=O)O)[C@H](O)[C@@H](O)[C@@H]1O]

wherein the wavy line marked with a single asterisk (*) indicates the site of covalent attachment to D or to a Spacer Unit (Y); and the wavy line marked with a double asterisk (**) indicates the point of covalent attachment to A, B or S*.

Embodiment 31. The Camptothecin-Linker compound of any one of embodiments 20-29 having formula (iii), formula (iv), formula (v) and formula (vi), wherein S* is a PEG group.

Embodiment 32. The Camptothecin-Linker compound of any one of embodiments 20-29 having formula (ii), formula (iv) or formula (vi), wherein the Spacer Unit (Y) has the formula of:

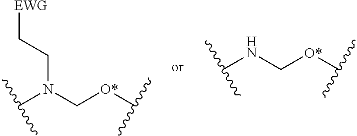

wherein EWG is an electron-withdrawing group;
O* represent the oxygen atom from a hydroxy functional group of D;
the wavy line adjacent to the nitrogen atom indicates the site of covalent attachment to the carbonyl carbon atom of the Glycoside (e.g., Glucuronide) Unit; and
the wavy line adjacent to O* indicates the site of covalent attachment to the remainder of D, or
the Spacer Unit (Y) has the formula of:

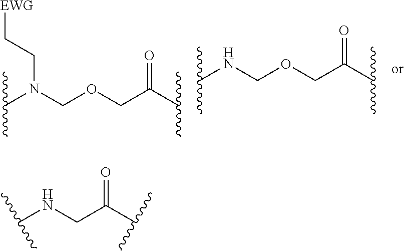

wherein
EWG is an electron-withdrawing group;
the wavy line adjacent to the nitrogen atom indicates the site of covalent attachment to the carbonyl carbon atom of the Glycoside (e.g., Glucuronide) Unit; and
the wavy line adjacent to the carbonyl carbon atom indicates the site of covalent attachment to the nitrogen atom of the amine functional group of D.

Embodiment 33. The Camptothecin-Linker Compound of any one of embodiments 20-29 having formula (vii), formula (viii) or formula (ix), wherein A is a Connector Unit, or having formula (i), formula (iii), formula (x) or formula (xi), wherein A is a Connector Unit and RL is a Releasable linker other than a Glycoside (e.g., Glucuronide) Unit.

Embodiment 34. The Camptothecin-Linker Compound of embodiment 33 having formula (i), formula (iii) or formula (x), wherein RL has the formula:

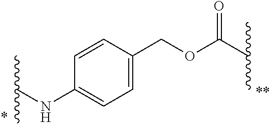 [SMILES: *Nc1ccc(COC(*)=O)cc1]

wherein
the wavy line marked with a double asterisk (**) indicates the site of covalent attachment to D; and
the wavy line marked with a single asterisk (*) indicates the point of covalent attachment to A, S* or W.

Embodiment 35. The Camptothecin-Linker Compound of embodiment 34 having formula (x) wherein W is an Amino Acid Unit selected from the group consisting of N-methyl-glycine (sarcosine), N-methyl-alanine, N-methyl-β-alanine, valine and N-methyl-valine.

Embodiment 36. The Camptothecin-Linker Compound of any one of embodiments 33-35 wherein Z′-A- is comprised of a maleimido-alkanoyl moiety or mDPR, the basic nitrogen atom of which is optionally protonated or protected by an acid-labile protecting group.

Embodiment 37. The Camptothecin-Linker Compound of any one of embodiments 33-35 having formula (iii) or formula (x), wherein
Z′-A- has a formula selected from the group consisting of:

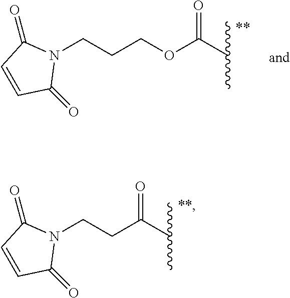 [SMILES: *C(=O)OCCCN1C(=O)C=CC1=O ; *C(=O)CCN1C(=O)C=CC1=O]

wherein the wavy line marked with a double asterisk (**) indicates the site of covalent attachment to S.

Embodiment 38. The Camptothecin-Linker Compound of any one of embodiments 33-35 having formula (iii) or formula (x), wherein
S* has the formula of:

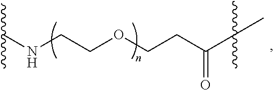

wherein subscript n is an integer ranging from 2 to 36.

Embodiment 39. The Camptothecin-Linker Compound of embodiment 33 or 34 of formula (viii) or formula (x) in which Z′-A-S*-W- has the formula of:

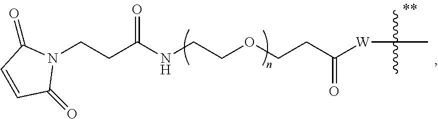

wherein subscript n is an integer ranging from 2 to 10, preferably ranging from 2 to 4; the wavy line marked with a double asterisk (**) indicates the site of covalent attachment to D or RL.

Embodiment 40. A method of treating cancer in a subject in need thereof, comprising administering to the subject an effective amount of a Camptothecin Conjugate of any one of embodiments 1-19, optionally said cancer is selected from the group consisting of lymphomas, leukemias, and solid tumors, optionally a lymphoma or a leukemia.

Embodiment 41. Use of a Camptothecin Conjugate of any one of embodiments 1-19 in preparation of a medicament for treatment of a cancer in a subject, optionally said cancer is selected from the group consisting of lymphomas, leukemias, and solid tumors, optionally a lymphoma or a leukemia.

Embodiment 42. A pharmaceutically acceptable composition comprising a Camptothecin Conjugate of any one of embodiments 1-19 and at least one pharmaceutically acceptable excipient.

Embodiment 43. A composition for treatment of a cancer in a subject in need thereof, wherein the composition is comprised of an effective amount of a Camptothecin Conjugate of any one of embodiments 1-19, optionally said cancer is selected from the group consisting of lymphomas, leukemias, and solid tumors, optionally a lymphoma or a leukemia.

| SEQ | | |
|---|---|---|
| ID | | |
| NO | Description | Sequence |
| 1 | cAC10 | DYYIT |
| | CDR-H1 | |
| 2 | cAC10 | WIYPGSGNTKYNEKFKG |
| | CDR-H2 | |
| 3 | cAC10 | YGNYWFAY |
| | CDR-H3 | |
| 4 | cAC10 | KASQSVDFDGDSYMN |
| | CDR-L1 | |
| 5 | cAC10 | AASNLES |
| | CDR-L2 | |
| 6 | cAC10 | QQSNEDPWT |
| | CDR-L3 | |
| 7 | cAC10 VH | QIQLQQSGPEVVKPGASVKISCKASGYTFTDYYITWVKQKPGQG |
| | | LEWIGWIYPGSGNTKY |
| | | NEKFKGKATLTVDTSSSTAFMQLSSLTSEDTAVYFCANYGNYW |
| | | FAYWGQGTQVTVSA |
| 8 | cAC10 VL | DIVLTQSPASLAVSLGQRATISCKASQSVDFDGDSYMNWYQQKP |
| | | GQPPKVLIYAASNLES |
| | | GIPARFSGSGSGTDFTLNIHPVEEEDAATYYCQQSNEDPWTFGGG |
| | | TKLEIK |
| 9 | cAC10 HC | QIQLQQSGPEVVKPGASVKISCKASGYTFTDYYITWVKQKPGQG |
| | | LEWIGWIYPGSGNTKY |
| | | NEKFKGKATLTVDTSSSTAFMQLSSLTSEDTAVYFCANYGNYW |
| | | FAYWGQGTQVTVSAAST |
| | | KGPSVFPLAPSSKSTSGGTAALGCLVKDYFPEPVTVSWNSGALTS |
| | | GVHTFPAVLQSS |
| | | GLYSLSSVVTVPSSSLGTQTYICNVNHKPSNTKVDKKVEPKSCD |
| | | KTHTCPPCPAPELLGG |
| | | PSVFLFPPKPKDTLMISRTPEVTCVVVDVSHEDPEVKFNWYVDG |
| | | VEVHNAKTKPREEQYN |
| | | STYRVVSVLTVLHQDWLNGKEYKCKVSNKALPAPIEKTISKAKG |
| | | QPREPQVYTLPPSRDE |
| | | LTKNQVSLTCLVKGFYPSDIAVEWESNGQPENNYKTTPPVLDSD |
| | | GSFFLYSKLTVDKSRW |
| | | QQGNVFSCSVMHEALHNHYTQKSLSLSPGK |
| 10 | cAC10 HC | QIQLQQSGPEVVKPGASVKISCKASGYTFTDYYITWVKQKPGQG |
| | v2 | LEWIGWIYPGSGNTKY |
| | | NEKFKGKATLTVDTSSSTAFMQLSSLTSEDTAVYFCANYGNYW |
| | | FAYWGQGTQVTVSAAST |
| | | KGPSVFPLAPSSKSTSGGTAALGCLVKDYFPEPVTVSWNSGALTS |
| | | GVHTFPAVLQSS |
| | | GLYSLSSVVTVPSSSLGTQTYICNVNHKPSNTKVDKKVEPKSCD |
| | | KTHTCPPCPAPELLGG |
| | | PSVFLFPPKPKDTLMISRTPEVTCVVVDVSHEDPEVKFNWYVDG |
| | | VEVHNAKTKPREEQYN |
| | | STYRVVSVLTVLHQDWLNGKEYKCKVSNKALPAPIEKTISKAKG |
| | | QPREPQVYTLPPSRDE |
| | | LTKNQVSLTCLVKGFYPSDIAVEWESNGQPENNYKTTPPVLDSD |
| | | GSFFLYSKLTVDKSRW |
| | | QQGNVFSCSVMHEALHNHYTQKSLSLSPG |
| 11 | cAC10 LC | DIVLTQSPASLAVSLGQRATISCKASQSVDFDGDSYMNWYQQKP |
| | | GQPPKVLIYAASNLES |
| | | GIPARFSGSGSGTDFTLNIHPVEEEDAATYYCQQSNEDPWTFGGG |
| | | TKLEIKR |
| | | TVAAPSVFIFPPSDEQLKSGTASVVCLLNNFYPREAKVQWKVDN |
| | | ALQSGNSQESVTEQDS |
| | | KDSTYSLSSTLTLSKADYEKHKVYACEVTHQGLSSPVTKSFNRG |
| | | EC |
| 12 | h1F6 VH | QVQLVQSGAEVKKPGASVKVSCKASGYTFTNYGMNWVRQAPG |
| | | QGLKWMGWINTYTGEPTY |
| | | ADAFKGRVTMTRDTSISTAYMELSRLRSDDTAVYYCARDYGDY |
| | | GMDYWGQGTTVTVSS |
| 13 | h1F6 VL | DIVMTQSPDSLAVSLGERATINCRASKSVSTSGYSFMHWYQQKP |
| | | GQPPKLLIYLASNLES |
| | | GVPDRFSGSGSGTDFTLTISSLQAEDVAVYYCQHSREVPWTFGQ |
| | | GTKVEIK |
| 14 | h1F6 HC | QVQLVQSGAEVKKPGASVKVSCKASGYTFTNYGMNWVRQAPG |
| | | QGLKWMGWINTYTGEPTY |
| | | ADAFKGRVTMTRDTSISTAYMELSRLRSDDTAVYYCARDYGDY |
| | | GMDYWGQGTTVTVSSAS |
| | | TKGPSVFPLAPSSKSTSGGTAALGCLVKDYFPEPVTVSWNSGAL |
| | | TSGVHTFPAVLQSSGL |
| | | YSLSSVVTVPSSSLGTQTYICNVNHKPSNTKVDKKVEPKSCDKT |
| | | HTCPPCPAPELLGGPS |
| | | VFLFPPKPKDTLMISRTPEVTCVVVDVSHEDPEVKFNWYVDGVE |
| | | VHNAKTKPREEQYNST |
| | | YRVVSVLTVLHQDWLNGKEYKCKVSNKALPAPIEKTISKAKGQ |
| | | PREPQVYTLPPSRDELT |
| | | KNQVSLTCLVKGFYPSDIAVEWESNGQPENNYKTTPPVLDSDGS |
| | | FFLYSKLTVDKSRWQQ |
| | | GNVFSCSVMHEALHNHYTQKSLSLSPGK |
| 15 | h1F6 LC | DIVMTQSPDSLAVSLGERATINCRASKSVSTSGYSFMHWYQQKP |
| | | GQPPKLLIYLASNLES |
| | | GVPDRFSGSGSGTDFTLTISSLQAEDVAVYYCQHSREVPWTFGQ |
| | | GTKVEIKRTVAAPSVF |
| | | IFPPSDEQLKSGTASVVCLLNNFYPREAKVQWKVDNALQSGNSQ |
| | | ESVTEQDSKDSTYSLS |
| | | STLTLSKADYEKHKVYACEVTHQGLSSPVTKSFNRGEC |
| 16 | TROP2 | NYGMN |
| | CDR-H1 | |
| 17 | TROP2 | WINTYTGEPTYTDDFKG |
| | CDR-H2 | |
| 18 | TROP2 | GGFGSSYWYFDV |
| | CDR-H3 | |
| 19 | TROP2 | KASQDVSIAVA |
| | CDR-L1 | |
| 20 | TROP2 | SASYRYT |
| | CDR-L2 | |
| 21 | TROP2 | QQHYITPLT |
| | CDR-L3 | |
| 22 | TROP2 VH | QVQLQQSGSELKKPGASVKVSCKASGYTFTNYGMNWVKQAPG |
| | | QGLKWMGWINTYTGEPT |
| | | YTDDFKGRFAFSLDTSVSTAYLQISSLKADDTAVYFCARGGFGSS |
| | | YWYFDVWGQGSLVTVSS |
| 23 | TROP2 VL | DIQLTQSPSSLSASVGDRVSITCKASQDVSIAVAWYQQKPGKAPK |
| | | LLIYSASYRYTGVP |
| | | DRFSGSGSGTDFTLTISSLQPEDFAVYYCQQHYITPLTFGAGTKV |
| | | EIK |
| 24 | TROP2 | TAGMQ |
| | CDR-H1 | |
| 25 | TROP2 | WINTHSGVPKYAEDFKG |
| | CDR-H2 | |
| 26 | TROP2 | SGFGSSYWYFDV |
| | CDR-H3 | |
| 27 | TROP2 | KASQDVSTAVA |
| | CDR-L1 | |
| 28 | TROP2 | SASYRYT |
| | CDR-L2 | |
| 29 | TROP2 | QQHYITPLT |
| | CDR-L3 | |
| 30 | TROP2 VH | QVQLVQSGAEVKKPGASVKVSCKASGYTFTTAGMQWVRQAPG |
| | | QGLEWMGWINTHSGVPKYAEDFKGRVTISADTSTSTAYLQLSSL |
| | | KSEDTAVYYCARSGFGSSYWYFDVWGQGTLVTVSS |
| 31 | TROP2 VL | DIQMTQSPSSLSASVGDRVTITCKASQDVSTAVAWYQQKPGKAP |
| | | KLLIYSASYRYTGVPSRFSGSGSGTDFTLTISSLQPEDFAVYYCQQ |
| | | HYITPLTFGQGTKLEIK |
| 32 | MICA CDR- | SQNIY |
| | H1 | |
| 33 | MICA CDR- | YIEPYNVVPMYNPKFKG |
| | H2 | |
| 34 | MICA CDR- | SGSSNFDY |
| | H3 | |
| 35 | MICA CDR- | SASSSISSHYLH |
| | L1 | |
| 36 | MICA CDR- | RTSNLAS |
| | L2 | |
| 37 | MICA CDR- | QQGSSLPLT |
| | L3 | |
| 38 | MICA VH | EIQLVQSGAEVKKPGASVKVSCKASGYAFTSQNIYWVRQAPGQ |
| | | GLEWIGYIEPYNVVPMYNPKFKGRATLTVDKSTSTAYLELSSLRS |
| | | EDTAVYYCARSGSSNFDYWGQGTLVTVSS |
| 39 | MICA VL | DIQLTQSPSSLSASVGDRVTITCSASSSISSHYLHWYQQKPGKSPK |
| | | LLIYRTSNLASGVPSRFSGSGSGTDYTLTISSLQPEDFATYYCQQG |
| | | SSLPLTFGQGTKVEIK |
| 40 | MICA CDR- | NYAMH |
| | H1 | |
| 41 | MICA CDR- | LIWYDGSNKFYGDSVKG |
| | H2 | |
| 42 | MICA CDR- | EGSGHY |
| | H3 | |
| 43 | MICA CDR- | RASQGISSALA |
| | L1 | |
| 44 | MICA CDR- | DASSLES |
| | L2 | |
| 45 | MICA CDR- | QQFNSYPIT |
| | L3 | |
| 46 | MICA VH | QVQLVESGGGVVQPGRSLRLSCAASGFTFSNYAMHWVRQAPGE |
| | | GLEWVALIWYDGSNKFYGDSVKGRFTISRDNSKNTLYLQMNSL |
| | | SAEDTAVYYCAREGSGHYWGQGTLVTVSS |
| 47 | MICA VL | AIQLTQSPSSLSASVGDRVTITCRASQGISSALAWYQQKPGKVPK |
| | | SLIYDASSLESGVPSRFSGSGSGTDFTLTISSLQPEDFATYYCQQF |
| | | NSYPITFGQGTRLEIK |
| 48 | MICA CDR- | NYAMS |
| | H1 | |
| 49 | MICA CDR- | YISPGGDYIYYADSVKG |
| | H2 | |
| 50 | MICA CDR- | DRRHYGSYAMDY |
| | H3 | |
| 51 | MICA CDR- | RSSKSLLHSNLNTYLY |
| | L1 | |
| 52 | MICA CDR- | RMSNLAS |
| | L2 | |
| 53 | MICA CDR- | MQHLEYPFT |
| | L3 | |
| 54 | MICA VH | QVQLVESGGGLVKPGGSLRLSCAASGFTFSNYAMSWIRQAPGK |
| | | GLEWVSYISPGGDYIYYADSVKGRFTISRDNAKNSLYLQMNSLR |
| | | AEDTAVYYCTTDRRHYGSYAMDYWGQGTLVTVSS |
| 55 | MICA VL | DIVMTQSPLSLPVTPGEPASISCRSSKSLLHSNLNTYLYWFLQKPG |
| | | QSPQILIYRMSNLASGVPDRFSGSGSGTAFTLKISRVEAEDVGVY |
| | | YCMQHLEYPFTFGPGTKLEIK |
| 56 | MICA CDR- | TYAFH |
| | H1 | |
| 57 | MICA CDR- | GIVPIFGTLKYAQKFQD |
| | H2 | |
| 58 | MICA CDR- | AIQLEGRPFDH |
| | H3 | |
| 59 | MICA CDR- | RASQGITSYLA |
| | L1 | |
| 60 | MICA CDR- | AASALQS |
| | L2 | |
| 61 | MICA CDR- | QQVNRGAAIT |
| | L3 | |
| 62 | MICA VH | QVQLVQSGAEVKKPGSSVRVSCRASGGSSTTYAFHWVRQAPGQ |
| | | GLEWMGGIVPIFGTLKYAQKFQDRVTLTADKSTGTAYMELNSL |
| | | RLDDTAVYYCARAIQLEGRPFDHWGQGTQVTVSA |
| 63 | MICA VL | DIQLTQSPSFLSASVGDRVTITCRASQGITSYLAWYQQKPGKAPK |
| | | LLIYAASALQSGVPSRFSGRGSGTEFTLTISSLQPEDFATYYCQQV |
| | | NRGAAITFGHGTRLDIK |
| 64 | CD24 CDR- | TYAFH |
| | H1 | |
| 65 | CD24 CDR- | GIVPIFGTLKYAQKFQD |
| | H2 | |
| 66 | CD24 CDR- | AIQLEGRPFDH |
| | H3 | |
| 67 | CD24 CDR- | RASQGITSYLA |
| | L1 | |
| 68 | CD24 CDR- | AASALQS |
| | L2 | |
| 69 | CD24 CDR- | QQVNRGAAIT |
| | L3 | |
| 70 | CD24 VH | QVQLVQSGAEVKKPGSSVRVSCRASGGSSTTYAFHWVRQAPGQ |
| | | GLEWMGGIVPIFGTLKYAQKFQDRVTLTADKSTGTAYMELNSL |
| | | RLDDTAVYYCARAIQLEGRPFDHWGQGTQVTVSA |
| 71 | CD24 VL | DIQLTQSPSFLSASVGDRVTITCRASQGITSYLAWYQQKPGKAPK |
| | | LLIYAASALQSGVPS |
| | | RFSGRGSGTEFTLTISSLQPEDFATYYCQQVNRGAAITFGHGTRL |
| | | DIK |
| 72 | ITGav CDR- | RYTMH |
| | H1 | |
| 73 | ITGav CDR- | VISFDGSNKYYVDSVKG |
| | H2 | |
| 74 | ITGav CDR- | EARGSYAFDI |
| | H3 | |
| 75 | ITGav CDR- | RASQSVSSYLA |
| | L1 | |
| 76 | ITGav CDR- | DASNRAT |
| | L2 | |
| 77 | ITGav CDR- | QQRSNWPPFT |
| | L3 | |
| 78 | ITGav VH | QVQLVESGGGVVQPGRSRRLSCAASGFTFSRYTMHWVRQAPGK |
| | | GLEWVAVISFDGSNKYYVDSVKGRFTISRDNSENTLYLQVNILR |
| | | AEDTAVYYCAREARGSYAFDIWGQGTMVTVSS |
| 79 | ITGav VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPPFTFGPGTKVDIK |
| 80 | ITGav CDR- | SFWMH |
| | H1 | |
| 81 | ITGav CDR- | YINPRSGYTEYNEIFRD |
| | H2 | |
| 82 | ITGav CDR- | FLGRGAMDY |
| | H3 | |
| 83 | ITGav CDR- | RASQDISNYLA |
| | L1 | |
| 84 | ITGav CDR- | YTSKIHS |
| | L2 | |
| 85 | ITGav CDR- | QQGNTFPYT |
| | L3 | |
| 86 | ITGav VH | QVQLQQSGGELAKPGASVKVSCKASGYTFSSFWMHWVRQAPG |
| | | QGLEWIGYINPRSGYTEYNEIFRDKATMTTDTSTSTAYMELSSLR |
| | | SEDTAVYYCASFLGRGAMDYWGQGTTVTVSS |
| 87 | ITGav VL | DIQMTQSPSSLSASVGDRVTITCRASQDISNYLAWYQQKPGKAP |
| | | KLLIYYTSKIHSGVPSRFSGSGSGTDYTFTISSLQPEDIATYYCQQ |
| | | GNTFPYTFGQGTKVEIK |
| 88 | gpA33 CDR- | TSSYYWG |
| | H1 | |
| 89 | gpA33 CDR- | TIYYNGSTYYSPSLKS |
| | H2 | |
| 90 | gpA33 CDR- | QGYDIKINIDV |
| | H3 | |
| 91 | gpA33 CDR- | RASQSVSSYLA |
| | L1 | |
| 92 | gpA33 CDR- | VASNRAT |
| | L2 | |
| 93 | gpA33 CDR- | QQRSNWPLT |
| | L3 | |
| 94 | gpA33 VH | QLQLQESGPGLVKPSETLSLTCTVSGGSISTSSYYWGWIRQPPGK |
| | | GLEWIGTIYYNGSTYYSPSLKSRVSISVDTSKNQFSLKLSSVTAA |
| | | DTSVYYCARQGYDIKINIDVWGQGTTVTVSS |
| 95 | gpA33 VL | EIVLTQSPATLSLSPGERATLSCRASQSVSSYLAWYQQKPGQAPR |
| | | LLIYVASNRATGIPARFSGSGSGTDFTLTISSLEPEDFAVYYCQQR |
| | | SNWPLTFGGGTKVEIK |
| 96 | IL1Rap | SSWMN |
| | CDR-H1 | |
| 97 | IL1Rap | RIYPGDGNTHYAQKFQG |
| | CDR-H2 | |
| 98 | IL1Rap | GYLDPMDY |
| | CDR-H3 | |
| 99 | IL1Rap | QASQGINNYLN |
| | CDR-L1 | |
| 100 | IL1Rap | YTSGLHA |
| | CDR-L2 | |
| 101 | IL1Rap | QQYSILPWT |
| | CDR-L3 | |
| 102 | IL1Rap VH | QVQLVQSGAEVKKPGSSVKVSCKASGYAFTSSWMNWVRQAPG |
| | | QGLEWMGRIYPGDGNTHYAQKFQGRVTLTADKSTSTAYMELSS |
| | | LRSEDTAVYYCGEGYLDPMDYWGQGTLVTVSS |
| 103 | IL1Rap VL | DIQMTQSPSSLSASVGDRVTITCQASQGINNYLNWYQQKPGKAP |
| | | KLLIHYTSGLHAGVPSRFSGSGSGTDYTLTISSLEPEDVATYYCQ |
| | | QYSILPWTFGGGTKVEIK |
| 104 | EpCAM | SYGMH |
| | CDR-H1 | |
| 105 | EpCAM | VISYDGSNKYYADSVKG |
| | CDR-H2 | |
| 106 | EpCAM | DMG |
| | CDR-H3 | |
| 107 | EpCAM | RTSQSISSYLN |
| | CDR-L1 | |
| 108 | EpCAM | WASTRES |
| | CDR-L2 | |
| 109 | EpCAM | QQSYDIPYT |
| | CDR-L3 | |
| 110 | EpCAM VH | EVQLLESGGGVVQPGRSLRLSCAASGFTFSSYGMHWVRQAPGK |
| | | GLEWVAVISYDGSNKYYADSVKGRFTISRDNSKNTLYLQMNSL |
| | | RAEDTAVYYCAKDMGWGSGWRPYYYYGMDVWGQGTTVTVS |
| | | S |
| 111 | EpCAM VL | ELQMTQSPSSLSASVGDRVTITCRTSQSISSYLNWYQQKPGQPPK |
| | | LLIYWASTRESGVPDRFSGSGSGTDFTLTISSLQPEDSATYYCQQS |
| | | YDIPYTFGQGTKLEIK |
| 112 | EpCAM | NYWMS |
| | CDR-H1 | |
| 113 | EpCAM | NIKQDGSEKFYADSVKG |
| | CDR-H2 | |
| 114 | EpCAM | VGPSWEQDY |
| | CDR-H3 | |
| 115 | EpCAM | TGSSSNIGSYYGVH |
| | CDR-L1 | |
| 116 | EpCAM | SDTNRPS |
| | CDR-L2 | |
| 117 | EpCAM | QSYDKGFGHRV |
| | CDR-L3 | |
| 118 | EpCAM VH | EVQLVESGGGLVQPGGSLRLSCAASGFTFSNYWMSWVRQAPGK |
| | | GLEWVANIKQDGSEKFYADSVKGRFTISRDNAKNSLYLQMNSL |
| | | RAEDTAVYYCARVGPSWEQDYWGQGTLVTVSA |
| 119 | EpCAM VL | QSVLTQPPSVSGAPGQRVTISCTGSSSNIGSYYGVHWYQQLPGTA |
| | | PKLLIYSDTNRPSGVPDRFSGSKSGTSASLAITGLQAEDEADYYC |
| | | QSYD |
| 120 | EpCAM | SYAIS |
| | CDR-H1 | |
| 121 | EpCAM | GIIPIFGTANYAQKFQG |
| | CDR-H2 | |
| 122 | EpCAM | GLLWNY |
| | CDR-H3 | |
| 123 | EpCAM | RASQSVSSNLA |
| | CDR-L1 | |
| 124 | EpCAM | GASTTAS |
| | CDR-L2 | |
| 125 | EpCAM | QQYNNWPPAYT |
| | CDR-L3 | |
| 126 | EpCAM VH | QVQLVQSGAEVKKPGSSVKVSCKASGGTFSSYAISWVRQAPGQ |
| | | GLEWMGGIIPIFGTANYAQKFQGRVTITADESTSTAYMELSSLRS |
| | | EDTAVYYCARGLLWNYWGQGTLVTVSS |
| 127 | EpCAM VL | EIVMTQSPATLSVSPGERATLSCRASQSVSSNLAWYQQKPGQAP |
| | | RLIIYGASTTASGIPARFSASGSGTDFTLTISSLQSEDFAVYYCQQ |
| | | YNNWPPAYTFGQGTKLEIK |
| 128 | EpCAM | NYGMN |
| | CDR-H1 | |
| 129 | EpCAM | WINTYTGEPTYGEDFKG |
| | CDR-H2 | |
| 130 | EpCAM | FGNYVDY |
| | CDR-H3 | |
| 131 | EpCAM | RSSKNLLHSNGITYLY |
| | CDR-L1 | |
| 132 | EpCAM | QMSNLAS |
| | CDR-L2 | |
| 133 | EpCAM | AQNLEIPRT |
| | CDR-L3 | |
| 134 | EpCAM VH | QVQLVQSGPEVKKPGASVKVSCKASGYTFTNYGMNWVRQAPG |
| | | QGLEWMGWINTYTGEPTYGEDFKGRFAFSLDTSASTAYMELSSL |
| | | RSEDTAVYFCARFGNYVDYWGQGSLVTVSS |
| 135 | EpCAM VL | DIVMTQSPLSLPVTPGEPASISCRSSKNLLHSNGITYLYWYLQKP |
| | | GQSPQLLIYQMSNLASGVPDRFSSSGSGTDFTLKISRVEAEDVGV |
| | | YYCAQNLEIPRTFGQGTKVEIK |
| 136 | EpCAM | KYGMN |
| | CDR-H1 | |
| 137 | EpCAM | WINTYTEEPTYGDDFKG |
| | CDR-H2 | |
| 138 | EpCAM | FGSAVDY |
| | CDR-H3 | |
| 139 | EpCAM | RSSKSLLHSNGITYLY |
| | CDR-L1 | |
| 140 | EpCAM | QMSNRAS |
| | CDR-L2 | |
| 141 | EpCAM | AQNLELPRT |
| | CDR-L3 | |
| 142 | EpCAM VH | QIQLVQSGPEVKKPGESVKISCKASGYTFTKYGMNWVKQAPGQ |
| | | GLKWMGWINTYTEEPTYGDDFKGRFTFTLDTSTSTAYLEISSLRS |
| | | EDTATYFCARFGSAVDYWGQGTLVTVSS |
| 143 | EpCAM VL | DIVMTQSALSNPVTLGESGSISCRSSKSLLHSNGITYLYWYLQKP |
| | | GQSPQLLIYQMSNRASGVPDRFSSSGSGTDFTLKISRVEAEDVGV |
| | | YYCAQNLELPRTFGQGTKLEMKR |
| 144 | EpCAM | DYSMH |
| | CDR-H1 | |
| 145 | EpCAM | WINTETGEPTYADDFKG |
| | CDR-H2 | |
| 146 | EpCAM | TAVY |
| | CDR-H3 | |
| 147 | EpCAM | RASQEISVSLS |
| | CDR-L1 | |
| 148 | EpCAM | ATSTLDS |
| | CDR-L2 | |
| 149 | EpCAM | LQYASYPWT |
| | CDR-L3 | |
| 150 | EpCAM VH | QVKLQESGPELKKPGETVKISCKASGYTFTDYSMHWVKQAPGK |
| | | GLKWMGWINTETGEPTYADDFKGRFAFSLETSASTAYLQINNLK |
| | | NEDTATYFCARTAVYWGQGTTVTVSS |
| 151 | EpCAM VL | DIQMTQSPSSLSASLGERVSLTCRASQEISVSLSWLQQEPDGTIKR |
| | | LIYATSTLDSGVPKRFSGSRSGSDYSLTISSLESEDFVDYYCLQYA |
| | | SYPWTFGGGTKLEIKR |
| 152 | CD352 | NYGMN |
| | CDR-H1 | |
| 153 | CD352 | WINTYSGEPRYADDFKG |
| | CDR-H2 | |
| 154 | CD352 | DYGRWYFDV |
| | CDR-H3 | |
| 155 | CD352 | RASSSVSHMH |
| | CDR-L1 | |
| 156 | CD352 | ATSNLAS |
| | CDR-L2 | |
| 157 | CD352 | QQWSSTPRT |
| | CDR-L3 | |
| 158 | CD352 VH | QIQLVQSGSELKKPGASVKVSCKASGYTFTNYGMNWVRQAPGQ |
| | | DLKWMGWINTYSGEPRYADDFKGRFVFSLDKSVNTAYLQISSL |
| | | KAEDTAVYYCARDYGRWYFDVWGQGTTVTVSS |
| 159 | CD352 VL | QIVLSQSPATLSLSPGERATMSCRASSSVSHMHWYQQKPGQAPR |
| | | PWIYATSNLASGVPARFSGSGSGTDYTLTISSLEPEDFAVYYCQQ |
| | | WSSTPRTFGGGTKVEIKR |
| 160 | CS1 CDR- | RYWMS |
| | H1 | |
| 161 | CS1 CDR- | EINPDSSTINYAPSLKD |
| | H2 | |
| 162 | CS1 CDR- | PDGNYWYFDV |
| | H3 | |
| 163 | CS1 CDR- | KASQDVGIAVA |
| | L1 | |
| 164 | CS1 CDR- | WASTRHT |
| | L2 | |
| 165 | CS1 CDR- | QQYSSYPYT |
| | L3 | |
| 166 | CS1 VH | EVQLVESGGGLVQPGGSLRLSCAASGFDFSRYWMSWVRQAPGK |
| | | GLEWIGEINPDSSTINYAPSLKDKFIISRDNAKNSLYLQMNSLRAE |
| | | DTAVYYCARPDGNYWYFDVWGQGTLVTVSS |
| 167 | CS1 VL | DIQMTQSPSSLSASVGDRVTITCKASQDVGIAVAWYQQKPGKVP |
| | | KLLIYWASTRHTGVPDRFSGSGSGTDFTLTISSLQPEDVATYYCQ |
| | | QYSSYPYTFGQGTKVEIKR |
| 168 | CD38 CDR- | SFAMS |
| | H1 | |
| 169 | CD38 CDR- | AISGSGGGTYYADSVKG |
| | H2 | |
| 170 | CD38 CDR- | DKILWFGEPVFDY |
| | H3 | |
| 171 | CD38 CDR- | RASQSVSSYLA |
| | L1 | |
| 172 | CD38 CDR- | DASNRAT |
| | L2 | |
| 173 | CD38 CDR- | QQRSNWPPT |
| | L3 | |
| 174 | CD38 VH | EVQLLESGGGLVQPGGSLRLSCAVSGFTFNSFAMSWVRQAPGK |
| | | GLEWVSAISGSGGGTYYADSVKGRFTISRDNSKNTLYLQMNSLR |
| | | AEDTAVYFCAKDKILWFGEPVFDYWGQGTLVTVSS |
| 175 | CD38 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPPTFGQGTKVEIKR |
| 176 | CD25 CDR- | SYRMH |
| | H1 | |
| 177 | CD25 CDR- | YINPSTGYTEYNQKFKD |
| | H2 | |
| 178 | CD25 CDR- | GGGVFDY |
| | H3 | |
| 179 | CD25 CDR- | SASSSISYMH |
| | L1 | |
| 180 | CD25 CDR- | TTSNLAS |
| | L2 | |
| 181 | CD25 CDR- | HQRSTYPLT |
| | L3 | |
| 182 | CD25 VH | QVQLVQSGAEVKKPGSSVKVSCKASGYTFTSYRMHWVRQAPG |
| | | QGLEWIGYINPSTGYTEYNQKFKDKATITADESTNTAYMELSSL |
| | | RSEDTAVYYCARGGGVFDYWGQGTLVTVSS |
| 183 | CD25 VL | DIQMTQSPSTLSASVGDRVTITCSASSSISYMHWYQQKPGKAPKL |
| | | LIYTTSNLASGVPARFSGSGSGTEFTLTISSLQPDDFATYYCHQRS |
| | | TYPLTFGQGTKVEVK |
| 184 | ADAM9 | SYWM |
| | CDR-H1 | |
| 185 | ADAM9 | EIIPINGHTNYNEKFKS |
| | CDR-H2 | |
| 186 | ADAM9 | GGYYYYGSRDYFDY |
| | CDR-H3 | |
| 187 | ADAM9 | KASQSVDYDGDSYMN |
| | CDR-L1 | |
| 188 | ADAM9 | AASDLES |
| | CDR-L2 | |
| 189 | ADAM9 | QQSHEDPFT |
| | CDR-L3 | |
| 190 | ADAM9 VH | QVQLQQPGAELVKPGASVKLSCKASGYTFTSYWMHWVKQRPG |
| | | QGLEWIGEIIPINGHTNYNEKFKSKATLTLDKSSSTAYMQLSSLA |
| | | SEDSAVYYCARGGYYYYGSRDYFDYWGQGTTLTVSS |
| 191 | ADAM9 VL | DIVLTQSPASLAVSLGQRATISCKASQSVDYDGDSYMNWYQQIP |
| | | GQPPKLLIYAASDLESGIPARFSGSGSGTDFTLNIHPVEEEDAATY |
| | | YCQQSHEDPFTFGGGTKLEIK |
| 192 | ADAM9 | SYWM |
| | CDR-H1 | |
| 193 | ADAM9 | EIIPIFGHTNYNEKFKS |
| | CDR-H2 | |
| 194 | ADAM9 | GGYYYYPRQGFLDY |
| | CDR-H3 | |
| 195 | ADAM9 | KASQSVDYDSGDSYMN |
| | CDR-L1 | |
| 196 | ADAM9 | AASDLES |
| | CDR-L2 | |
| 197 | ADAM9 | QQSHEDPFT |
| | CDR-L3 | |
| 198 | ADAM9 VH | EVQLVESGGGLVKPGGSLRLSCAASGFTFSSYWMHWVRQAPGK |
| | | GLEWVGEIIPIFGHTNYNEKFKSRFTISLDNSKNTLYLQMGSLRA |
| | | EDTAVYYCARGGYYYYPRQGFLDYWGQGTTVTVSS |
| 199 | ADAM9 VL | DIVMTQSPDSLAVSLGERATISCKASQSVDYSGDSYMNWYQQK |
| | | PGQPPKLLIYAASDLESGIPARFSGSGSGTDFTLTISSLEPEDFATY |
| | | YCQQSHEDPFTFGQGTKLEIK |
| 200 | CD59 CDR- | YGMN |
| | H1 | |
| 201 | CD59 CDR- | YISSSSSTIYADSVKG |
| | H2 | |
| 202 | CD59 CDR- | GPGMDV |
| | H3 | |
| 203 | CD59 CDR- | KSSQSVLYSSNNKNYLA |
| | L1 | |
| 204 | CD59 CDR- | WASTRES |
| | L2 | |
| 205 | CD59 CDR- | QQYYSTPQLT |
| | L3 | |
| 206 | CD59 VH | QVQLQQSGGGVVQPGRSLGLSCAASFTFSSYGMNWVRQAPGKG |
| | | LEWVSYISSSSSTIYADSVKGRFTISRDNSKNTLYLQMNSLRAED |
| | | TAVYYCARGPGMDVWGQGTTVTVS |
| 207 | CD59 VL | DIVLTQSPDSLAVSLGERATINCKSSQSVLYSSNNKNYLAWYQQ |
| | | KPGQPPKLLIYWASTRESGVPDRFSGSGSGTDFTPAISSLQAEDV |
| | | AVYYCQQYYSTPQLTFGGGTKVDIK |
| 208 | CD19 CDR- | TSGMGVG |
| | H1 | |
| 209 | CD19 CDR- | HIWWDDDKRYNPALKS |
| | H2 | |
| 210 | CD19 CDR- | MELWSYYFDY |
| | H3 | |
| 211 | CD19 CDR- | SASSSVSYMH |
| | L1 | |
| 212 | CD19 CDR- | DTSKLAS |
| | L2 | |
| 213 | CD19 CDR- | FQGSVYPFT |
| | L3 | |
| 214 | CD19 VH | QVQLQESGPGLVKPSQTLSLTCTVSGGSISTSGMGVGWIRQHPG |
| | | KGLEWIGHIWWDDDKRYNPALKSRVTISVDTSKNQFSLKLSSVT |
| | | AADTAVYYCARMELWSYYFDYWGQGTLVTVSS |
| 215 | CD19 VL | EIVLTQSPATLSLSPGERATLSCSASSSVSYMHWYQQKPGQAPRL |
| | | LIYDTSKLASGIPARFSGSGSGTDFTLTISSLEPEDVAVYYCFQGS |
| | | VYPFTFGQGTKLEIKR |
| 216 | CD70 CDR- | NYGMN |
| | H1 | |
| 217 | CD70 CDR- | WINTYTGEPTYADAFKG |
| | H2 | |
| 218 | CD70 CDR- | DYGDYGMDY |
| | H3 | |
| 219 | CD70 CDR- | RASKSVSTSGYSFMH |
| | L1 | |
| 220 | CD70 CDR- | LASNLES |
| | L2 | |
| 221 | CD70 CDR- | QHSREVPWT |
| | L3 | |
| 222 | CD70 VH | QVQLVQSGAEVKKPGASVKVSCKASGYTFTNYGMNWVRQAPG |
| | | QGLKWMGWINTYTGEPTYADAFKGRVTMTRDTSISTAYMELSR |
| | | LRSDDTAVYYCARDYGDYGMDYWGQGTTVTVSS |
| 223 | CD70 VL | DIVMTQSPDSLAVSLGERATINCRASKSVSTSGYSFMHWYQQKP |
| | | GQPPKLLIYLASNLESGVPDRFSGSGSGTDFTLTISSLQAEDVAVY |
| | | YCQHSREVPWTFGQGTKVEIK |
| 224 | B7H4 CDR- | SGYSWH |
| | H1 | |
| 225 | B7H4 CDR- | YIHSSGSTNYNPSLKS |
| | H2 | |
| 226 | B7H4 CDR- | YDDYFEY |
| | H3 | |
| 227 | B7H4 CDR- | KASQNVGFNVA |
| | L1 | |
| 228 | B7H4 CDR- | SASYRYS |
| | L2 | |
| 229 | B7H4 CDR- | QQYNWYPFT |
| | L3 | |
| 230 | B7H4 VH | EVQLQESGPGLVKPSETLSLTCAVTGYSITSGYSWHWIRQFPGNG |
| | | LEWMGYIHSSGSTNYNPSLKSRISISRDTSKNQFFLKLSSVTAADT |
| | | AVYYCAGYDDYFEYWGQGTTVTVSS |
| 231 | B7H4 VL | DIQMTQSPSSLSASVGDRVTITCKASQNVGFNVAWYQQKPGKSP |
| | | KALIYSASYRYSGVPSRFSGSGSGTDFTLTISSLQPEDFAEYFCQQ |
| | | YNWYPFTFGQGTKLEIK |
| 232 | CD138 | NYWIE |
| | CDR-H1 | |
| 233 | CD138 | EILPGTGRTIYNEKFKG |
| | CDR-H2 | |
| 234 | CD138 | RDYYGNFYYAMDY |
| | CDR-H3 | |
| 235 | CD138 | SASQGINNYLN |
| | CDR-L1 | |
| 236 | CD138 | YTSTLQS |
| | CDR-L2 | |
| 237 | CD138 | QQYSKLPRT |
| | CDR-L3 | |
| 238 | CD138 VH | QVQLQQSGSELMMPGASVKISCKATGYTFSNYWIEWVKQRPGH |
| | | GLEWIGEILPGTGRTIY |
| | | NEKFKGKATFTADISSNTVQMQLSSLTSEDSAVYYCARRDYYGN |
| | | FYYAMDYWGQGTSVTVSS |
| 239 | CD138 VL | DIQMTQSTSSLSASLGDRVTISCSASQGINNYLNWYQQKPDGTV |
| | | ELLIYYTSTLQSGVP |
| | | SRFSGSGSGTDYSLTISNLEPEDIGTYYCQQYSKLPRTFGGGTKLE |
| | | IK |
| 240 | CD166 | TYGMGVG |
| | CDR-H1 | |
| 241 | CD166 | NIWWSEDKHYSPSLKS |
| | CDR-H2 | |
| 242 | CD166 | IDYGNDYAFTY |
| | CDR-H3 | |
| 243 | CD166 | RSSKSLLHSNGITYLY |
| | CDR-L1 | |
| 244 | CD166 | QMSNLAS |
| | CDR-L2 | |
| 245 | CD166 | AQNLELPYT |
| | CDR-L3 | |
| 246 | CD166 VH | QITLKESGPTLVKPTQTLTLTCTFSGFSLSTYGMGVGWIRQPPGK |
| | | ALEWLANIWWSEDKHYSPSLKSRLTITKDTSKNQVVLTITNVDP |
| | | VDTATYYCVQIDYGNDYAFTYWGQGTLVTVSS |
| 247 | CD166 VL | DIVMTQSPLSLPVTPGEPASISCRSSKSLLHSNGITYLYWYLQKPG |
| | | QSPQLLIYQMSNLASGVPDRFSGSGSGTDFTLKISRVEAEDVGVY |
| | | YCAQNLELPYTFGQGTKLEIK |
| 248 | CD51 CDR- | RYTMH |
| | H1 | |
| 249 | CD51 CDR- | VISFDGSNKYYVDSVKG |
| | H2 | |
| 250 | CD51 CDR- | EARGSYAFDI |
| | H3 | |
| 251 | CD51 CDR- | RASQSVSSYLA |
| | L1 | |
| 252 | CD51 CDR- | DASNRAT |
| | L2 | |
| 253 | CD51 CDR- | QQRSNWPPFT |
| | L3 | |
| 254 | CD51 VH | QVQLVESGGGVVQPGRSRRLSCAASGFTFSRYTMHWVRQAPGK |
| | | GLEWVAVISFDGSNKYYVDSVKGRFTISRDNSENTLYLQVNILR |
| | | AEDTAVYYCAREARGSYAFDIWGQGTMVTVSS |
| 255 | CD51 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPPFTFGPGTKVDIK |
| 256 | CD56 CDR- | SFGMH |
| | H1 | |
| 257 | CD56 CDR- | YISSGSFTIYYADSVKG |
| | H2 | |
| 258 | CD56 CDR- | MRKGYAMDY |
| | H3 | |
| 259 | CD56 CDR- | RSSQIIIHSDGNTYLE |
| | L1 | |
| 260 | CD56 CDR- | KVSNRFS |
| | L2 | |
| 261 | CD56 CDR- | FQGSHVPHT |
| | L3 | |
| 262 | CD56 VH | QVQLVESGGGVVQPGRSLRLSCAASGFTFSSFGMHWVRQAPGK |
| | | GLEWVAYISSGSFTIYYADSVKGRFTISRDNSKNTLYLQMNSLRA |
| | | EDTAVYYCARMRKGYAMDYWGQGTLVTVSS |
| 263 | CD56 VL | DVVMTQSPLSLPVTLGQPASISCRSSQIIIHSDGNTYLEWFQQRPG |
| | | QSPRRLIYKVSNRFSGVPDRFSGSGSGTDFTLKISRVEAEDVGVY |
| | | YCFQGSHVPHTFGQGTKVEIK |
| 264 | CD74 CDR- | NYGVN |
| | H1 | |
| 265 | CD74 CDR- | WINPNTGEPTFDDDFKG |
| | H2 | |
| 266 | CD74 CDR- | SRGKNEAWFAY |
| | H3 | |
| 267 | CD74 CDR- | RSSQSLVHRNGNTYLH |
| | L1 | |
| 268 | CD74 CDR- | TVSNRFS |
| | L2 | |
| 269 | CD74 CDR- | SQSSHVPPT |
| | L3 | |
| 270 | CD74 VH | QVQLQQSGSELKKPGASVKVSCKASGYTFTNYGVNWIKQAPGQ |
| | | GLQWMGWINPNTGEPTFDDDFKGRFAFSLDTSVSTAYLQISSLK |
| | | ADDTAVYFCSRSRGKNEAWFAYWGQGTLVTVSS |
| 271 | CD74 VL | DIQLTQSPLSLPVTLGQPASISCRSSQSLVHRNGNTYLHWFQQRP |
| | | GQSPRLLIYTVSNRFSGVPDRFSGSGSGTDFTLKISRVEAEDVGV |
| | | YFCSQSSHVPPTFGAGTRLEIK |
| 272 | CEACAM5 | TYWMS |
| | CDR-H1 | |
| 273 | CEACAM5 | EIHPDSSTINYAPSLKD |
| | CDR-H2 | |
| 274 | CEACAM5 | LYFGFPWFAY |
| | CDR-H3 | |
| 275 | CEACAM5 | KASQDVGTSVA |
| | CDR-L1 | |
| 276 | CEACAM5 | WTSTRHT |
| | CDR-L2 | |
| 277 | CEACAM5 | QQYSLYRS |
| | CDR-L3 | |
| 278 | CEACAM5 | EVQLVESGGGVVQPGRSLRLSCSASGFDFTTYWMSWVRQAPGK |
| | VH | GLEWIGEIHPDSSTINYAPSLKDRFTISRDNAKNTLFLQMDSLRPE |
| | | DTGVYFCASLYFGFPWFAYWGQGTPVTVSS |
| 279 | CEACAM5 | DIQLTQSPSSLSASVGDRVTITCKASQDVGTSVAWYQQKPGKAP |
| | VL | KLLIYWTSTRHTGVPSRFSGSGSGTDFTFTISSLQPEDIATYYCQQ |
| | | YSLYRSFGQGTKVEIK |
| 280 | CanAg | YYGMN |
| | CDR-H1 | |
| 281 | CanAg | WIDTTTGEPTYAQKFQG |
| | CDR-H2 | |
| 282 | CanAg | RGPYNWYFDV |
| | CDR-H3 | |
| 283 | CanAg | RSSKSLLHSNGNTYLY |
| | CDR-L1 | |
| 284 | CanAg | RMSNLVS |
| | CDR-L2 | |
| 285 | CanAg | LQHLEYPFT |
| | CDR-L3 | |
| 286 | CanAg VH | QVQLVQSGAEVKKPGETVKISCKASDYTFTYYGMNWVKQAPG |
| | | QGLKWMGWIDTTTGEPTYAQKFQGRIAFSLETSASTAYLQIKSL |
| | | KSEDTATYFCARRGPYNWYFDVWGQGTTVTVSS |
| 287 | CanAg VL | DIVMTQSPLSVPVTPGEPVSISCRSSKSLLHSNGNTYLYWFLQRP |
| | | GQSPQLLIYRMSNLVSGVPDRFSGSGSGTAFTLRISRVEAEDVGV |
| | | YYCLQHLEYPFTFGPGTKLELK |
| 288 | DLL-3 CDR- | NYGMN |
| | H1 | |
| 289 | DLL-3 CDR- | WINTYTGEPTYADDFKG |
| | H2 | |
| 290 | DLL-3 CDR- | IGDSSPSDY |
| | H3 | |
| 291 | DLL-3 CDR- | KASQSVSNDVV |
| | L1 | |
| 292 | DLL-3 CDR- | YASNRYT |
| | L2 | |
| 293 | DLL-3 CDR- | QQDYTSPWT |
| | L3 | |
| 294 | DLL-3 VH | QVQLVQSGAEVKKPGASVKVSCKASGYTFTNYGMNWVRQAPG |
| | | QGLEWMGWINTYTGEPTY |
| | | ADDFKGRVTMTTDTSTSTAYMELRSLRSDDTAVYYCARIGDSSP |
| | | SDYWGQGTLVTVSS |
| 295 | DLL-3 VL | EIVMTQSPATLSVSPGERATLSCKASQSVSNDVVWYQQKPGQAP |
| | | RLLIYYASNRYTGIPA |
| | | RFSGSGSGTEFTLTISSLQSEDFAVYYCQQDYTSPWTFGQGTKLE |
| | | IK |
| 296 | DPEP-3 | SYWIE |
| | CDR-H1 | |
| 297 | DPEP-3 | EILPGSGNTYYNERFKD |
| | CDR-H2 | |
| 298 | DPEP-3 | RAAAYYSNPEWFAY |
| | CDR-H3 | |
| 299 | DPEP-3 | TASSSVNSFYLH |
| | CDR-L1 | |
| 300 | DPEP-3 | STSNLAS |
| | CDR-L2 | |
| 301 | DPEP-3 | HQYHRSPYT |
| | CDR-L3 | |
| 302 | DPEP-3 VH | QVQLVQSGAEVKKPGSSVKVSCKASGGTFSSYWIEWVRQAPGQ |
| | | GLEWMGEILPGSGNTYYNERFKDRVTITADESTSTAYMELSSLR |
| | | SEDTAVYYCARRAAAYYSNPEWFAYWGQGTLVTVSS |
| 303 | DPEP-3 VL | EIVLTQSPATLSLSPGERATLSCTASSSVNSFYLHWYQQKPGLAP |
| | | RLLIYSTSNLASGIPDRFSGSGSGTDFTLTISRLEPEDFAVYYCHQ |
| | | YHRSPYTFGQGTKLEIK |
| 304 | EGFR CDR- | SYWMQ |
| | H1 | |
| 305 | EGFR CDR- | TIYPGDGDTTYTQKFQG |
| | H2 | |
| 306 | EGFR CDR- | YDAPGYAMDY |
| | H3 | |
| 307 | EGFR CDR- | RASQDINNYLA |
| | L1 | |
| 308 | EGFR CDR- | YTSTLHP |
| | L2 | |
| 309 | EGFR CDR- | LQYDNLLYT |
| | L3 | |
| 310 | EGFR VH | QVQLVQSGAEVAKPGASVKLSCKASGYTFTSYWMQWVKQRPG |
| | | QGLECIGTIYPGDGDTTYTQKFQGKATLTADKSSSTAYMQLSSL |
| | | RSEDSAVYYCARYDAPGYAMDYWGQGTLVTVSS |
| 311 | EGFR VL | DIQMTQSPSSLSASVGDRVTITCRASQDINNYLAWYQHKPGKGP |
| | | KLLIHYTSTLHPGIPSRFSGSGSGRDYSFSISSLEPEDIATYYCLQY |
| | | DNLLYTFGQGTKLEIK |
| 312 | EGFR CDR- | RDFAWN |
| | H1 | |
| 313 | EGFR CDR- | YISYNGNTRYQPSLKS |
| | H2 | |
| 314 | EGFR CDR- | ASRGFPY |
| | H3 | |
| 315 | EGFR CDR- | HSSQDINSNIG |
| | L1 | |
| 316 | EGFR CDR- | HGTNLDD |
| | L2 | |
| 317 | EGFR CDR- | VQYAQFPWT |
| | L3 | |
| 318 | EGFR VH | EVQLQESGPGLVKPSQTLSLTCTVSGYSISRDFAWNWIRQPPGKG |
| | | LEWMGYISYNGNTRYQPSLKSRITISRDTSKNQFFLKLNSVTAAD |
| | | TATYYCVTASRGFPYWGQGTLVTVSS |
| 319 | EGFR VL | DIQMTQSPSSMSVSVGDRVTITCHSSQDINSNIGWLQQKPGKSFK |
| | | GLIYHGTNLDDGVPSRFSGSGSGTDYTLTISSLQPEDFATYYCVQ |
| | | YAQFPWTFGGGTKLEIK |
| 320 | EGFR CDR- | RDFAWN |
| | H1 | |
| 321 | EGFR CDR- | YISYNGNTRYQPSLKS |
| | H2 | |
| 322 | EGFR CDR- | ASRGFPY |
| | H3 | |
| 323 | EGFR CDR- | HSSQDINSNIG |
| | L1 | |
| 324 | EGFR CDR- | HGTNLDD |
| | L2 | |
| 325 | EGFR CDR- | VQYAQFPWT |
| | L3 | |
| 326 | EGFR VH | EVQLQESGPGLVKPSQTLSLTCTVSGYSISRDFAWNWIRQPPGKG |
| | | LEWMGYISYNGNTRYQPSLKSRITISRDTSKNQFFLKLNSVTAAD |
| | | TATYYCVTASRGFPYWGQGTLVTVSS |
| 327 | EGFR VL | DIQMTQSPSSMSVSVGDRVTITCHSSQDINSNIGWLQQKPGKSFK |
| | | GLIYHGTNLDDGVPSRFSGSGSGTDYTLTISSLQPEDFATYYCVQ |
| | | YAQFPWTFGGGTKLEIK |
| 328 | EGFR CDR- | NYGVH |
| | H1 | |
| 329 | EGFR CDR- | VIWSGGNTDYNTPFTS |
| | H2 | |
| 330 | EGFR CDR- | ALTYYDYEFAY |
| | H3 | |
| 331 | EGFR CDR- | RASQSIGTNIH |
| | L1 | |
| 332 | EGFR CDR- | YASESIS |
| | L2 | |
| 333 | EGFR CDR- | QQNNNWPTT |
| | L3 | |
| 334 | EGFR VH | QVQLKQSGPGLVQPSQSLSITCTVSGFSLTNYGVHWVRQSPGKG |
| | | LEWLGVIWSGGNTDYNTPFTSRLSINKDNSKSQVFFKMNSLQSN |
| | | DTAIYYCARALTYYDYEFAYWGQGTLVTVSA |
| 335 | EGFR VL | DILLTQSPVILSVSPGERVSFSCRASQSIGTNIHWYQQRTNGSPRL |
| | | LIKYASESISGIPSRFSGSGSGTDFTLSINSVESEDIADYYCQQNNN |
| | | WPTTFGAGTKLELK |
| 336 | FRa CDR- | GYFMN |
| | H1 | |
| 337 | FRa CDR- | RIHPYDGDTFYNQKFQG |
| | H2 | |
| 338 | FRa CDR- | YDGSRAMDY |
| | H3 | |
| 339 | FRa CDR-L1 | KASQSVSFAGTSLMH |
| 340 | FRa CDR-L2 | RASNLEA |
| 341 | FRa CDR-L3 | QQSREYPYT |
| 342 | FRa VH | QVQLVQSGAEVVKPGASVKISCKASGYTFTGYFMNWVKQSPGQ |
| | | SLEWIGRIHPYDGDTFY |
| | | NQKFQGKATLTVDKSSNTAHMELLSLTSEDFAVYYCTRYDGSR |
| | | AMDYWGQGTTVTVSS |
| 343 | FRa VL | DIVLTQSPLSLAVSLGQPAIISCKASQSVSFAGTSLMHWYHQKPG |
| | | QQPRLLIYRASNLEAGVPDRFSGSGSKTDFTLTISPVEAEDAATY |
| | | YCQQSREYPYTFGGGTKLEIK |
| 344 | FRa CDR- | GYGLS |
| | H1 | |
| 345 | FRa CDR- | MISSGGSYTYYADSVKG |
| | H2 | |
| 346 | FRa CDR- | HGDDPAWFAY |
| | H3 | |
| 347 | FRa CDR-L1 | SVSSSISSNNLH |
| 348 | FRa CDR-L2 | GTSNLAS |
| 349 | FRa CDR-L3 | QQWSSYPYMYT |
| 350 | FRa VH | EVQLVESGGGVVQPGRSLRLSCSASGFTFSGYGLSWVRQAPGKG |
| | | LEWVAMISSGGSYTYY |
| | | ADSVKGRFAISRDNAKNTLFLQMDSLRPEDTGVYFCARHGDDP |
| | | AWFAYWGQGTPVTVSS |
| 351 | FRa VL | DIQLTQSPSSLSASVGDRVTITCSVSSSISSNNLHWYQQKPGKAPK |
| | | PWIYGTSNLASGVPSRFSGSGSGTDYTFTISSLQPEDIATYYCQQ |
| | | WSSYPYMYTFGQGTKVEIK |
| 352 | MUC-1 | NYWMN |
| | CDR-H1 | |
| 353 | MUC-1 | EIRLKSNNYTTHYAESVKG |
| | CDR-H2 | |
| 354 | MUC-1 | HYYFDY |
| | CDR-H3 | |
| 355 | MUC-1 | RSSKSLLHSNGITYFF |
| | CDR-L1 | |
| 356 | MUC-1 | QMSNLAS |
| | CDR-L2 | |
| 357 | MUC-1 | AQNLELPPT |
| | CDR-L3 | |
| 358 | MUC-1 VH | EVQLVESGGGLVQPGGSMRLSCVASGFPFSNYWMNWVRQAPG |
| | | KGLEWVGEIRLKSNNYTTHYAESVKGRFTISRDDSKNSLYLQMN |
| | | SLKTEDTAVYYCTRHYYFDYWGQGTLVTVSS |
| 359 | MUC-1 VL | DIVMTQSPLSNPVTPGEPASISCRSSKSLLHSNGITYFFWYLQKPG |
| | | QSPQLLIYQMSNLASGVPDRFSGSGSGTDFTLRISRVEAEDVGVY |
| | | YCAQNLELPPTFGQGTKVEIK |
| 360 | Mesothelin | SYWIG |
| | CDR-H1 | |
| 361 | Mesothelin | IIDPGDSRTRYSPSFQG |
| | CDR-H2 | |
| 362 | Mesothelin | GQLYGGTYMDG |
| | CDR-H3 | |
| 363 | Mesothelin | TGTSSDIGGYNSVS |
| | CDR-L1 | |
| 364 | Mesothelin | GVNNRPS |
| | CDR-L2 | |
| 365 | Mesothelin | SSYDIESATPV |
| | CDR-L3 | |
| 366 | Mesothelin | QVELVQSGAEVKKPGESLKISCKGSGYSFTSYWIGWVRQAPGKG |
| | VH | LEWMGIIDPGDSRTRYSPSFQGQVTISADKSISTAYLQWSSLKAS |
| | | DTAMYYCARGQLYGGTYMDGWGQGTLVTVSS |
| 367 | Mesothelin | DIALTQPASVSGSPGQSITISCTGTSSDIGGYNSVSWYQQHPGKAP |
| | VL | KLMIYGVNNRPSGV |
| | | SNRFSGSKSGNTASLTISGLQAEDEADYYCSSYDIESATPVFGGG |
| | | TKLTVL |
| 368 | ROR-1 | AYNIH |
| | CDR-H1 | |
| 369 | ROR-1 | SFDPYDGGSSYNQKFKD |
| | CDR-H2 | |
| 370 | ROR-1 | GWYYFDY |
| | CDR-H3 | |
| 371 | ROR-1 | RASKSISKYLA |
| | CDR-L1 | |
| 372 | ROR-1 | SGSTLQS |
| | CDR-L2 | |
| 373 | ROR-1 | QQHDESPYT |
| | CDR-L3 | |
| 374 | ROR-1 VH | QVQLQESGPGLVKPSQTLSLTCTVSGYAFTAYNIHWVRQAPGQG |
| | | LEWMGSFDPYDGGSSYNQKFKDRLTISKDTSKNQVVLTMTNMD |
| | | PVDTATYYCARGWYYFDYWGHGTLVTVSS |
| 375 | ROR-1 VL | DIVMTQTPLSLPVTPGEPASISCRASKSISKYLAWYQQKPGQAPR |
| | | LLIYSGSTLQSGIPPRFSGSGYGTDFTLTINNIESEDAAYYFCQQH |
| | | DESPYTFGEGTKVEIK |
| 376 | B7H4 CDR- | GSIKSGSYYWG |
| | H1 | |
| 377 | B7H4 CDR- | NIYYSGSTYYNPSLRS |
| | H2 | |
| 378 | B7H4 CDR- | AREGSYPNQFDP |
| | H3 | |
| 379 | B7H4 CDR- | RASQSVSSNLA |
| | L1 | |
| 380 | B7H4 CDR- | GASTRAT |
| | L2 | |
| 381 | B7H4 CDR- | QQYHSFPFT |
| | L3 | |
| 382 | B7H4 VH | QLQLQESGPGLVKPSETLSLTCTVSGGSIKSGSYYWGWIRQPPGK |
| | | GLEWIGNIYYSGSTY |
| | | YNPSLRSRVTISVDTSKNQFSLKLSSVTAADTAVYYCAREGSYPN |
| | | QFDPWGQGTLVTVSS |
| 383 | B7H4 VL | EIVMTQSPATLSVSPGERATLSCRASQSVSSNLAWYQQKPGQAP |
| | | RLLIYGASTRATGIPA |
| | | RFSGSGSGTEFTLTISSLQSEDFAVYYCQQYHSFPFTFGGGTKVEI |
| | | K |
| 384 | B7-H3 CDR- | SFGMH |
| | H1 | |
| 385 | B7-H3 CDR- | YISSDSSAIYY |
| | H2 | |
| 386 | B7-H3 CDR- | GRENIYYGSRLD |
| | H3 | |
| 387 | B7-H3 CDR- | KASQNVD |
| | L1 | |
| 388 | B7-H3 CDR- | SASYRYSGVPD |
| | L2 | |
| 389 | B7-H3 CDR- | QQYNNYPFTFGS |
| | L3 | |
| 390 | B7-H3 VH | DVQLVESGGGLVQPGGSRKLSCAASGFTFSSFGMHWVRQAPEK |
| | | GLEWVAYISSDSSAIYY |
| | | ADTVKGRFTISRDNPKNTLFLQMTSLRSEDTAMYYCGRGRENIY |
| | | YGSRLDYWGQGTTLTVSS |
| 391 | B7-H3 VL | DIAMTQSQKFMSTSVGDRVSVTCKASQNVDTNVAWYQQKPGQ |
| | | SPKALIYSASYRYSGVPD |
| | | RFTGSGSGTDFTLTINNVQSEDLAEYFCQQYNNYPFTFGSGTKLE |
| | | IK |
| 392 | B7-H3 CDR- | SYWMQWVRQA |
| | H1 | |
| 393 | B7-H3 CDR- | TIYPGDGDTRY |
| | H2 | |
| 394 | B7-H3 CDR- | RGIPRLWYFDVM |
| | H3 | |
| 395 | B7-H3 CDR- | ITCRASQDIS |
| | L1 | |
| 396 | B7-H3 CDR- | YTSRLHSGVPS |
| | L2 | |
| 397 | B7-H3 CDR- | QQGNTLPPFTGG |
| | L3 | |
| 398 | B7-H3 VH | DVQLVESGGGLVQPGGSRKLSCAASGFTFSSFGMHWVRQAPEK |
| | | GLEWVAYISSDSSAIYY |
| | | ADTVKGRFTISRDNPKNTLFLQMTSLRSEDTAMYYCGRGRENIY |
| | | YGSRLDYWGQGTTLTVSS |
| 399 | B7-H3 VL | DIAMTQSQKFMSTSVGDRVSVTCKASQNVDTNVAWYQQKPGQ |
| | | SPKALIYSASYRYSGVPD |
| | | RFTGSGSGTDFTLTINNVQSEDLAEYFCQQYNNYPFTFGSGTKLE |
| | | IK |
| 400 | B7-H3 CDR- | SYGMSWVRQA |
| | H1 | |
| 401 | B7-H3 CDR- | INSGGSNTYY |
| | H2 | |
| 402 | B7-H3 CDR- | HDGGAMDYW |
| | H3 | |
| 403 | B7-H3 CDR- | ITCRASESIYSYLA |
| | L1 | |
| 404 | B7-H3 CDR- | NTKTLPE |
| | L2 | |
| 405 | B7-H3 CDR- | HHYGTPPWTFG |
| | L3 | |
| 406 | B7-H3 VH | EVQLVESGGGLVKPGGSLRLSCAASGFTFSSYGMSWVRQAPGK |
| | | GLEWVATINSGGSNTYY |
| | | PDSLKGRFTISRDNAKNSLYLQMNSLRAEDTAVYYCARHDGGA |
| | | MDYWGQGTTVTVSS |
| 407 | B7-H3 VL | DIQMTQSPSSLSASVGDRVTITCRASESIYSYLAWYQQKPGKAPK |
| | | LLVYNTKTLPEGVPSRFSGSGSGTDFTLTISSLQPEDFATYYCQH |
| | | HYGTPPWTFGQGTRLEIK |
| 408 | B7-H3 CDR- | SFGMHWVRQA |
| | H1 | |
| 409 | B7-H3 CDR- | ISSGSGTIYYADTVKGRFTI |
| | H2 | |
| 410 | B7-H3 CDR- | HGYRYEGFDYWG |
| | H3 | |
| 411 | B7-H3 CDR- | ITCKASQNVDTNVA |
| | L1 | |
| 412 | B7-H3 CDR- | SASYRYSGVPS |
| | L2 | |
| 413 | B7-H3 CDR- | QQYNNYPFTFGQ |
| | L3 | |
| 414 | B7-H3 VH | EVQLVESGGGLVQPGGSLRLSCAASGFTFSSFGMHWVRQAPGK |
| | | GLEWVAYISSGSGTIY |
| | | YADTVKGRFTISRDNAKNSLYLQMNSLRAEDTAVYYCARHGYR |
| | | YEGFDYWGQGTTVTVSS |
| 415 | B7-H3 VL | DIQMTQSPSFLSASVGDRVTITCKASQNVDTNVAWYQQKPGKA |
| | | PKALIYSASYRYSGVPSRFSGSGSGTDFTLTISSLQPEDFAEYFCQ |
| | | QYNNYPFTFGQGTKLEIK |
| 416 | B7-H3 CDR- | NYVMH |
| | H1 | |
| 417 | B7-H3 CDR- | YINPYNDDVKYNEKFKG |
| | H2 | |
| 418 | B7-H3 CDR- | WGYYGSPLYYFDY |
| | H3 | |
| 419 | B7-H3 CDR- | RASSRLIYMH |
| | L1 | |
| 420 | B7-H3 CDR- | ATSNLAS |
| | L2 | |
| 421 | B7-H3 CDR- | QQWNSNPPT |
| | L3 | |
| 422 | B7-H3 VH | EVQLQQSGPELVKPGASVKMSCKASGYTFTNYVMHWVKQKPG |
| | | QGLEWIGYINPYNDDVKYNEKFKGKATQTSDKSSSTAYMELSSL |
| | | TSEDSAVYYCARWGYYGSPLYYFDYWGQGTTLTVSS |
| 423 | B7-H3 VL | QIVLSQSPTILSASPGEKVTMTCRASSRLIYMHWYQQKPGSSPKP |
| | | WIYATSNLASGVPAR |
| | | FSGSGSGTSYSLTISRVEAEDAATYYCQQWNSNPPTFGTGTKLEL |
| | | K |
| 424 | B7-H3 CDR- | NYVMH |
| | H1 | |
| 425 | B7-H3 CDR- | YINPYNDDVKYNEKFKG |
| | H2 | |
| 426 | B7-H3 CDR- | WGYYGSPLYYFDY |
| | H3 | |
| 427 | B7-H3 CDR- | RASSRLIYMH |
| | L1 | |
| 428 | B7-H3 CDR- | ATSNLAS |
| | L2 | |
| 429 | B7-H3 CDR- | QQWNSNPPT |
| | L3 | |
| 430 | B7-H3 VH | QVQLVQSGAEVKKPGSSVKVSCKASGYTFTNYVMHWVRQAPG |
| | | QGLEWMGYINPYNDDVKYNE |
| | | KFKGRVTITADESTSTAYMELSSLRSEDTAVYYCARWGYYGSPL |
| | | YYFDYWGQGTLVTVSS |
| 431 | B7-H3 VL | EIVLTQSPATLSLSPGERATLSCRASSRLIYMHWYQQKPGQAPRP |
| | | LIYATSNLASGIPARFSGSGSGTDFTLTISSLEPEDFAVYYCQQWN |
| | | SNPPTFGQGTKVEIK |
| 432 | B7-H3 CDR- | GYSFTSYTIH |
| | H1 | |
| 433 | B7-H3 CDR- | YINPNSRNTDYAQKFQG |
| | H2 | |
| 434 | B7-H3 CDR- | YSGSTPYWYFDV |
| | H3 | |
| 435 | B7-H3 CDR- | RASSSVSYMN |
| | L1 | |
| 436 | B7-H3 CDR- | ATSNLAS |
| | L2 | |
| 437 | B7-H3 CDR- | QQWSSNPLT |
| | L3 | |
| 438 | B7-H3 VH | EVQLVQSGAEVKKPGSSVKVSCKASGYSFTSYTIHWVRQAPGQ |
| | | GLEWMGYINPNSRNTDYAQKFQGRVTLTADKSTSTAYMELSSL |
| | | RSEDTAVYYCARYSGSTPYWYFDVWGQGTTVTVSS |
| 439 | B7-H3 VL | DIQMTQSPSSLSASVGDRVTITCKASQNVGFNVAWYQQKPGKSP |
| | | KALIYSASYRYSGVPSRFSGSGSGTDFTLTISSLQPEDFAEYFCQQ |
| | | YNWYPFTFGQGTKLEIK |
| 440 | B7-H3 CDR- | GYTFSSYWMH |
| | H1 | |
| 441 | B7-H3 CDR- | LIHPDSGSTNYNEMFKN |
| | H2 | |
| 442 | B7-H3 CDR- | GGRLYFD |
| | H3 | |
| 443 | B7-H3 CDR- | RSSQSLVHSNGDTYLR |
| | L1 | |
| 444 | B7-H3 CDR- | KVSNRFS |
| | L2 | |
| 445 | B7-H3 CDR- | SQSTHVPYT |
| | L3 | |
| 446 | B7-H3 VH | EVQLVQSGAEVKKPGSSVKVSCKASGYTFSSYWMHWVRQAPG |
| | | QGLEWIGLIHPDSGSTNYNEMFKNRATLTVDRSTSTAYVELSSLR |
| | | SEDTAVYFCAGGGRLYFDYWGQGTTVTVSS |
| 447 | B7-H3 VL | DVVMTQSPLSLPVTPGEPASISCRSSQSLVHSNGDTYLRWYLQKP |
| | | GQSPQLLIYKVSNRFSGVPDRFSGSGSGTDFTLKISRVEAEDVGV |
| | | YYCSQSTHVPYTFGGGTKVEIK |
| 448 | B7-H3 CDR- | GYTFSSYWMH |
| | H1 | |
| 449 | B7-H3 CDR- | LIHPESGSTNYNEMFKN |
| | H2 | |
| 450 | B7-H3 CDR- | GGRLYFDY |
| | H3 | |
| 451 | B7-H3 CDR- | RSSQSLVHSNQDTYLR |
| | L1 | |
| 452 | B7-H3 CDR- | KVSNRFS |
| | L2 | |
| 453 | B7-H3 CDR- | SQSTHVPYT |
| | L3 | |
| 454 | B7-H3 VH | EVQLVQSGAEVKKPGSSVKVSCKASGYTFSSYWMHWVRQAPG |
| | | QGLEWIGLIHPESGSTNY |
| | | NEMFKNRATLTVDRSTSTAYMELSSLRSEDTAVYYCAGGGRLY |
| | | FDYWGQGTTVTVSS |
| 455 | B7-H3 VL | DIVMTQSPLSLPVTPGEPASISCRSSQSLVHSNQDTYLRWYLQKP |
| | | GQSPQLLIYKVSNRF |
| | | SGVPDRFSGSGSGTDFTLKKISRVEAEDVGVYYCSQSTHVPYTFG |
| | | GGTKVEIK |
| 456 | B7-H3 CDR- | TGYSITSGYSWH |
| | H1 | |
| 457 | B7-H3 CDR- | YIHSSGSTNYNPSLKS |
| | H2 | |
| 458 | B7-H3 CDR- | YDDYFEY |
| | H3 | |
| 459 | B7-H3 CDR- | KASQNVGFNVAW |
| | L1 | |
| 460 | B7-H3 CDR- | SASYRYS |
| | L2 | |
| 461 | B7-H3 CDR- | QQYNWYPFT |
| | L3 | |
| 462 | B7-H3 VH | EVQLQESGPGLVKPSETLSLTCAVTGYSITSGYSWHWIRQFPGNG |
| | | LEWMGYIHSSGSTNY |
| | | NPSLKSRISISRDTSKNQFFLKLSSVTAADTAVYYCAGYDDYFEY |
| | | WGQGTTVTVSS |
| 463 | B7-H3 VL | DIQMTQSPSSLSASVGDRVTITCKASQNVGGFNVAWYQQKPGKS |
| | | PKALIYSASYRYSGV |
| | | PSRFSGSGSGTDFTLTISSLQPEDFAEYFCQQYNWYPFTFGQGTK |
| | | LEIK |
| 464 | B7-H3 CDR- | NYDIN |
| | H1 | |
| 465 | B7-H3 CDR- | WIGWIFPGDDSTQYNEKFKG |
| | H2 | |
| 466 | B7-H3 CDR- | QTTGTWFAY |
| | H3 | |
| 467 | B7-H3 CDR- | RASQSISDYLY |
| | L1 | |
| 468 | B7-H3 CDR- | YASQSIS |
| | L2 | |
| 469 | B7-H3 CDR- | CQNGHSFPL |
| | L3 | |
| 470 | B7-H3 VH | QVQLVQSGAEVVKPGASVKLSCKTSGYTFTNYDINWVRQRPGQ |
| | | GLEWIGWIFPGDDSTQY |
| | | NEKFKGKATLTTDTSTSTAYMELSSLRSEDTAVYFCARQTTGTW |
| | | FAYWGQGTLVTVSS |
| 471 | B7-H3 VL | EIVMTQSPATLSVSPGERVTLSCRASQSISDYLYWYQQKSHESPR |
| | | LLIKYASQSISGIPA |
| | | RFSGSGSGSEFTLTINSVEPEDVGVYYCQNGHSFPLTFGQGTKLE |
| | | LK |
| 472 | B7-H3 VH | QVQLQQSGAEVKKPGSSVKVSCKASGGTFSSYAISWVRQAPGQ |
| | | GLEWMGGIIPILGIAN |
| | | YAQKFQGRVTITADESTSTAYMELSSLRSEDTAVYYCARGGSGS |
| | | YHMDVWGKGTTVTVSS |
| 473 | B7-H3 VL | EIVLTQSPATLSLSPGERATLSCRASQSVSSYLAWYQQKPGQAPR |
| | | LLIYDASNRATGIP |
| | | ARFSGSGSGTDFTLTISSLEPEDFAVYYCQQRSNWPPRITFGQGT |
| | | RLEIK |
| 474 | B7-H3 CDR- | TYNVH |
| | H1 | |
| 475 | B7-H3 CDR- | TIFPGNGDTSYNQKFKD |
| | H2 | |
| 476 | B7-H3 CDR- | WDDGNVGFAH |
| | H3 | |
| 477 | B7-H3 CDR- | RASENINNYLT |
| | L1 | |
| 478 | B7-H3 CDR- | HAKTLAE |
| | L2 | |
| 479 | B7-H3 CDR- | QHHYGTPPT |
| | L3 | |
| 480 | B7-H3 VH | QVQLQQPGAELVKPGASVKMSCKASGYTFTIYNVHWIKQTPGQ |
| | | GLEWMGTIFPGNGDTSY |
| | | NQKFKDKATLTTDKSSKTAYMQLNSLTSEDSAVYYCARWDDG |
| | | NVGFAHWGQGTLVTVSA |
| 481 | B7-H3 VL | DIQMTQSPASLSASVGETVTITCRASENINNYLTWFQQKQGKSPQ |
| | | LLVYHAKTLAEGVPS |
| | | RFSGSGSGTQFSLKINSLQPEDFGSYYCQHHYGTPPTFGGGTKLEI |
| | | K |
| 482 | B7-H3 VH | EVQLVQSGAEVKKPGASVKVSCKASGYTFTIYNVHWVRQAPGQ |
| | | GLEWMGTIFPGNGDTS |
| | | YNQKFKDKVTMTTDTSTSTAYMELSSLRSEDTAVYYCARWDD |
| | | GNVGFAHWGQGTLVTVSS |
| 483 | B7-H3 VL | DIQMTQSPSSLSASVGDRVTITCRASENINNYLTWFQQKQGKSPQ |
| | | LLIYHAKTLAEGVP |
| | | SRFSGSGSGTDFTLTISSLQPEDFATYYCQHHYGTPPTFGGGTKV |
| | | EIK |
| 484 | B7-H3 VH | EVQLVQSGAEVKKPGASVKVSCKASGYTFTIYNVHWIRQAPGQ |
| | | GLEWMGTIFPGNGDTSY |
| | | NQKFKDRATLTTDKSTKTAYMELRSLRSDDTAVYYCARWDDG |
| | | NVGFAHWGQGTLVTVSS |
| 485 | B7-H3 VL | DIQMTQSPSSLSASVGDRVTITCRASENINNYLTWFQQKPGKAPK |
| | | LLVYHAKTLAEGVPS |
| | | RFSGSGSGTQFTLTISSLQPEDFATYYCQHHYGTPPTFGQGTKLEI |
| | | K |
| 486 | HER3 H | QVQLQQWGAGLLKPSETLSLTCAVYGGSFSGYYWSWIRQPPGK |
| | | GLEWIGEINHSGSTNYN |
| | | PSLKSRVTISVETSKNQFSLKLSSVTAADTAVYYCARDKWTWYF |
| | | DLWGRGTLVTVSSAST |
| | | KGPSVFPLAPSSKSTSGGTAALGCLVKDYFPEPVTVSWNSGALTS |
| | | GVHTFPAVLQSSGL |
| | | YSLSSVVTVPSSSLGTQTYICNVNHKPSNTKVDKRVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | REEMTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 487 | HER3 L | DIEMTQSPDSLAVSLGERATINCRSSQSVLYSSSNRNYLAWYQQ |
| | | NPGQPPKLLIYWASTRESGVPDRFSGSGSGTDFTLTISSLQAEDV |
| | | AVYYCQQYYSTPRTFGQGTKVEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 488 | HER3 H | EVQLLESGGGLVQPGGSLRLSCAASGFTFSHYVMAWVRQAPGK |
| | | GLEWVSSISSSGGWTLY |
| | | ADSVKGRFTISRDNSKNTLYLQMNSLRAEDTAVYYCTRGLKMA |
| | | TIFDYWGQGTLVTVSSA |
| | | STKGPSVFPLAPCSRSTSESTAALGCLVKDYFPEPVTVSWNSGAL |
| | | TSGVHTFPAVLQSSG |
| | | LYSLSSVVTVPSSNFGTQTYTCNVDHKPSNTKVDKTVERKCCVE |
| | | CPPCPAPPVAGPSVFL |
| | | FPPKPKDTLMISRTPEVTCVVVDVSHEDPEVQFNWYVDGVEVH |
| | | NAKTKPREEQFNSTFRV |
| | | VSVLTVVHQDWLNGKEYKCKVSNKGLPAPIEKTISKTKGQPREP |
| | | QVYTLPPSREEMTKNQ |
| | | VSLTCLVKGFYPSDIAVEWESNGQPENNYKTTPPMLDSDGSFFL |
| | | YSKLTVDKSRWQQGNV |
| | | FSCSVMHEALHNHYTQKSLSLSPGK |
| 489 | HER3 L | QSALTQPASVSGSPGQSITISCTGTSSDVGSYNVVSWYQQHPGKA |
| | | PKLIIYEVSQRPSGVSNRFSGSKSGNTASLTISGLQTEDEADYYCC |
| | | SYAGSSIFVIFGGGTKVTVLGQPKAAPSVTLFPPSSEELQANKATL |
| | | VCLVSDFYPGAVTVAWKADGSPVKVGVETTKPSKQSNNKYAAS |
| | | SYLSLTPEQWKSHRSYSCRVTHEGSTVEKTVAPAECS |
| 490 | HER3 H | EVQLLESGGGLVQPGGSLRLSCAASGFTFSSYAMSWVRQAPGK |
| | | GLEWVSAINSQGKSTYYADSVKGRFTISRDNSKNTLYLQMNSLR |
| | | AEDTAVYYCARWGDEGFDIWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 491 | HER3 L | DIQMTQSPSSLSASVGDRVTITCRASQGISNWLAWYQQKPGKAP |
| | | KLLIYGASSLQSGVPSRFSGSGSGTDFTLTISSLQPEDFATYYCQQ |
| | | YSSFPTTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 492 | HER3 H | QVQLVQSGAEVKKPGASVKVSCKASGYTFRSSYISWVRQAPGQ |
| | | GLEWMGWIYAGTGSPSYNQKLQGRVTMTTDTSTSTAYMELRSL |
| | | RSDDTAVYYCARHRDYYSNSLTYWGQGTLVTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 493 | HER3 L | DIVMTQSPDSLAVSLGERATINCKSSQSVLNSGNQKNYLTWYQQ |
| | | KPGQPPKLLIYWASTRESGVPDRFSGSGSGTDFTLTISSLQAEDV |
| | | AVYYCQSDYSYPYTFGQGTKLEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 494 | PTK7 CDR- | TSNMGVG |
| | H1 | |
| 495 | PTK7 CDR- | HIWWDDDKYYSPSLKS |
| | H2 | |
| 496 | PTK7 CDR- | SNYGYAWFAY |
| | H3 | |
| 497 | PTK7 CDR- | KASQDIYPYLN |
| | L1 | |
| 498 | PTK7 CDR- | RTNRLLD |
| | L2 | |
| 499 | PTK7 CDR- | LQYDEFPLT |
| | L3 | |
| 500 | PTK7 VH | QITLKESGPTLVKPTQTLTLTCTFSGFSLSTSNMGVGWIRQPPGK |
| | | ALEWLAHIWWDDDKYYSPSLKSRLTITKDTSKNQVVLTMTNMD |
| | | PVDTATYYCVRSNYGYAWFAYWGQGTLVTVSS |
| 501 | PTK7 VL | DIQMTQSPSSLSASVGDRVTITCKASQDIYPYLNWFQQKPGKAP |
| | | KTLIYRTNRLLDGVPS |
| | | RFSGSGSGTDFTFTISSLQPEDIATYYCLQYDEFPLTFGAGTKLEI |
| | | K |
| 502 | PTK7 CDR- | DYAVH |
| | H1 | |
| 503 | PTK7 CDR- | VISTYNDYTYNNQDFKG |
| | H2 | |
| 504 | PTK7 CDR- | GNSYFYALDY |
| | H3 | |
| 505 | PTK7 CDR- | RASESVDSYGKSFMH |
| | L1 | |
| 506 | PTK7 CDR- | RASNLES |
| | L2 | |
| 507 | PTK7 CDR- | QQSNEDPWT |
| | L3 | |
| 508 | PTK7 VH | QVQLVQSGPEVKKPGASVKVSCKASGYTFTDYAVHWVRQAPG |
| | | KRLEWIGVISTYNDYTY |
| | | NNQDFKGRVTMTRDTSASTAYMELSRLRSEDTAVYYCARGNSY |
| | | FYALDYWGQGTSVTVSS |
| 509 | PTK7 VL | EIVLTQSPATLSLSPGERATLSCRASESVDSYGKSFMHWYQQKP |
| | | GQAPRLLIYRASNLES |
| | | GIPARFSGSGSGTDFTLTISSLEPEDFAVYYCQQSNEDPWTFGGG |
| | | TKLEIK |
| 510 | PTK7 CDR- | RYWMS |
| | H1 | |
| 511 | PTK7 CDR- | DLNPDSSAINYVDSVKG |
| | H2 | |
| 512 | PTK7 CDR- | ITTLVPYTMDF |
| | H3 | |
| 513 | PTK7 CDR- | ITNTDIDDDMN |
| | L1 | |
| 514 | PTK7 CDR- | EGNGLRP |
| | L2 | |
| 515 | PTK7 CDR- | LQSDNLPLT |
| | L3 | |
| 516 | PTK7 VH | EVQLVESGGGLVQPGGSLRLSCAASGFDFSRYWMSWVRQAPGK |
| | | GLEWIGDLNPDSSAINY |
| | | VDSVKGRFTISRDNAKNSLYLQMNSLRAEDTAVYYCTLITTLVP |
| | | YTMDFWGQGTSVTVSS |
| 517 | PTK7 VL | ETTLTQSPAFMSATPGDKVNISCITNTDIDDDMNWYQQKPGEAA |
| | | ILLISEGNGLRPGIPPRFSGSGYGTDFTLTINNIESEDAAYYFCLQS |
| | | DNLPLTFGSGTKLEIK |
| 518 | LIV1 CDR- | DYYMH |
| | H1 | |
| 519 | LIV1 CDR- | WIDPENGDTEYGPKFQG |
| | H2 | |
| 520 | LIV1 CDR- | HNAHYGTWFAY |
| | H3 | |
| 521 | LIV1 CDR- | RSSQSLLHSSGNTYLE |
| | L1 | |
| 522 | LIV1 CDR- | KISTRFS |
| | L2 | |
| 523 | LIV1 CDR- | FQGSHVPYT |
| | L3 | |
| 524 | LIV1 VH | QVQLVQSGAEVKKPGASVKVSCKASGLTIEDYYMHWVRQAPG |
| | | QGLEWMGWIDPENGDTEY |
| | | GPKFQGRVTMTRDTSINTAYMELSRLRSDDTAVYYCAVHNAHY |
| | | GTWFAYWGQGTLVTVSS |
| 525 | LIV1 VL | DVVMTQSPLSLPVTLGQPASISCRSSQSLLHSSGNTYLEWYQQRP |
| | | GQSPRPLIYKISTRFSGVPDRFSGSGSGTDFTLKISRVEAEDVGVY |
| | | YCFQGSHVPYTFGGGTKVEIK |
| 526 | avb6 CDR- | DYNVN |
| | H1 | |
| 527 | avb6 CDR- | VINPKYGTTRYNQKFKG |
| | H2 | |
| 528 | avb6 CDR- | GLNAWDY |
| | H3 | |
| 529 | avb6 CDR- | GASENIYGALN |
| | L1 | |
| 530 | avb6 CDR- | GATNLED |
| | L2 | |
| 531 | avb6 CDR- | QNVLTTPYT |
| | L3 | |
| 532 | avb6 VH | QFQLVQSGAEVKKPGASVKVSCKASGYSFTDYNVNWVRQAPG |
| | | QGLEWIGVINPKYGTTRY |
| | | NQKFKGRATLTVDKSTSTAYMELSSLRSEDTAVYYCTRGLNAW |
| | | DYWGQGTLVTVSS |
| 533 | avb6 VL | DIQMTQSPSSLSASVGDRVTITCGASENIYGALNWYQQKPGKAP |
| | | KLLIYGATNLEDGVPS |
| | | RFSGSGSGRDYTFTISSLQPEDIATYYCQNVLTTPYTFGQGTKLEI |
| | | K |
| 534 | avb6 CDR- | GYFMN |
| | H1 | |
| 535 | avb6 CDR- | LINPYNGDSFYNQKFKG |
| | H2 | |
| 536 | avb6 CDR- | GLRRDFDY |
| | H3 | |
| 537 | avb6 CDR- | KSSQSLLDSDGKTYLN |
| | L1 | |
| 538 | avb6 CDR- | LVSELDS |
| | L2 | |
| 539 | avb6 CDR- | WQGTHFPRT |
| | L3 | |
| 540 | avb6 VH | QVQLVQSGAEVKKPGASVKVSCKASGYSFSGYFMNWVRQAPG |
| | | QGLEWMGLINPYNGDSFY |
| | | NQKFKGRVTMTRQTSTSTVYMELSSLRSEDTAVYYCVRGLRRD |
| | | FDYWGQGTLVTVSS |
| 541 | avb6 VL | DVVMTQSPLSLPVTLGQPASISCKSSQSLLDSDGKTYLNWLFQRP |
| | | GQSPRRLIYLVSELD |
| | | SGVPDRFSGSGSGTDFTLKISRVEAEDVGVYYCWQGTHFPRTFG |
| | | GGTKLEIK |
| 542 | CD48 CDR- | DFGMN |
| | H1 | |
| 543 | CD48 CDR- | WINTFTGEPSYGNVFKG |
| | H2 | |
| 544 | CD48 CDR- | RHGNGNVFDS |
| | H3 | |
| 545 | CD48 CDR- | RASQSIGSNIH |
| | L1 | |
| 546 | CD48 CDR- | YTSESIS |
| | L2 | |
| 547 | CD48 CDR- | QQSNSWPLT |
| | L3 | |
| 548 | CD48 VH | QVQLVQSGSELKKPGASVKVSCKASGYTFTDFGMNWVRQAPG |
| | | QGLEWMGWINTFTGEPSYGNVFKGRFVFSLDTSVSTAYLQISSL |
| | | KAEDTAVYYCARRHGNGNVFDSWGQGTLVTVSS |
| 549 | CD48 VL | EIVLTQSPDFQSVTPKEKVTITCRASQSIGSNIHWYQQKPDQSPKL |
| | | LIKYTSESISGVPSRFSGSGSGTDFTLTINSLEAEDAATYYCQQSN |
| | | SWPLTFGGGTKVEIKR |
| 550 | PD-L1 CDR- | TAAIS |
| | H1 | |
| 551 | PD-L1 CDR- | GIIPIFGKAHYAQKFQG |
| | H2 | |
| 552 | PD-L1 CDR- | KFHFVSGSPFGMDV |
| | H3 | |
| 553 | PD-L1 CDR- | RASQSVSSYLA |
| | L1 | |
| 554 | PD-L1 CDR- | DASNRAT |
| | L2 | |
| 555 | PD-L1 CDR- | QQRSNWPT |
| | L3 | |
| 556 | PD-L1 VH | QVQLVQSGAEVKKPGSSVKVSCKTSGDTFSTAAISWVRQAPGQ |
| | | GLEWMGGIIPIFGKAHYAQKFQGRVTITADESTSTAYMELSSLRS |
| | | EDTAVYFCARKFHFVSGSPFGMDVWGQGTTVTVSS |
| 557 | PD-L1 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPTFGQGTKVEIK |
| 558 | IGF-1R | SYAIS |
| | CDR-H1 | |
| 559 | IGF-1R | GIIPIFGTANYAQKFQG |
| | CDR-H2 | |
| 560 | IGF-1R | APLRFLEWSTQDHYYYYYMDV |
| | CDR-H3 | |
| 561 | IGF-1R | QGDSLRSYYAT |
| | CDR-L1 | |
| 562 | IGF-1R | GENKRPS |
| | CDR-L2 | |
| 563 | IGF-1R | KSRDGSGQHLV |
| | CDR-L3 | |
| 564 | IGF-1R VH | EVQLVQSGAEVKKPGSSVKVSCKASGGTFSSYAISWVRQAPGQ |
| | | GLEWMGGIIPIFGTANY |
| | | AQKFQGRVTITADKSTSTAYMELSSLRSEDTAVYYCARAPLRFL |
| | | EWSTQDHYYYYYMDVWGKGTTVTVSS |
| 565 | IGF-1R VL | SSELTQDPAVSVALGQTVRITCQGDSLRSYYATWYQQKPGQAPI |
| | | LVIYGENKRPSGIPDR |
| | | FSGSSSGNTASLTITGAQAEDEADYYCKSRDGSGQHLVFGGGTK |
| | | LTVL |
| 566 | Claudin-18.2 | SYWIN |
| | CDR-H1 | |
| 567 | Claudin-18.2 | NIYPSDSYTNYNQKFKD |
| | CDR-H2 | |
| 568 | Claudin-18.2 | SWRGNSFDY |
| | CDR-H3 | |
| 569 | Claudin-18.2 | KSSQSLLNSGNQKNYLT |
| | CDR-L1 | |
| 570 | Claudin-18.2 | WASTRES |
| | CDR-L2 | |
| 571 | Claudin-18.2 | QNDYSYPFT |
| | CDR-L3 | |
| 572 | Claudin-18.2 | QVQLQQPGAELVRPGASVKLSCKASGYTFTSYWINWVKQRPGQ |
| | VH | GLEWIGNIYPSDSYTN |
| | | YNQKFKDKATLTVDKSSSTAYMQLSSPTSEDSAVYYCTRSWRG |
| | | NSFDYWGQGTTLTVSS |
| 573 | Claudin-18.2 | DIVMTQSPSSLTVTAGEKVTMSCKSSQSLLNSGNQKNYLTWYQ |
| | VL | QKPGQPPKLLIYWASTR |
| | | ESGVPDRFTGSGSGTDFTLTISSVQAEDLAVYYCQNDYSYPFTFG |
| | | SGTKLEIK |
| 574 | Claudin-18.2 | NYGMN |
| | CDR-H1 | |
| 575 | Claudin-18.2 | WINTNTGEPTYAEEFKG |
| | CDR-H2 | |
| 576 | Claudin-18.2 | LGFGNAMDY |
| | CDR-H3 | |
| 577 | Claudin-18.2 | KSSQSLLNSGNQKNYLT |
| | CDR-L1 | |
| 578 | Claudin-18.2 | WASTRES |
| | CDR-L2 | |
| 579 | Claudin-18.2 | QNDYSYPLT |
| | CDR-L3 | |
| 580 | Claudin-18.2 | QIQLVQSGPELKKPGETVKISCKASGYTFTNYGMNWVKQAPGK |
| | VH | GLKWMGWINTNTGEPTY |
| | | AEEFKGRFAFSLETSASTAYLQINNLKNEDTATYFCARLGFGNA |
| | | MDYWGQGTSVTVSS |
| 581 | Claudin-18.2 | DIVMTQSPSSLTVTAGEKVTMSCKSSQSLLNSGNQKNYLTWYQ |
| | VL | QKPGQPPKLLIYWASTR |
| | | ESGVPDRFTGSGSGTDFTLTISSVQAEDLAVYYCQNDYSYPLTFG |
| | | AGTKLELK |
| 582 | Nectin-4 | SYNMN |
| | CDR-H1 | |
| 583 | Nectin-4 | YISSSSSTIYYADSVKG |
| | CDR-H2 | |
| 584 | Nectin-4 | AYYYGMDV |
| | CDR-H3 | |
| 585 | Nectin-4 | RASQGISGWLA |
| | CDR-L1 | |
| 586 | Nectin-4 | AASTLQS |
| | CDR-L2 | |
| 587 | Nectin-4 | QQANSFPPT |
| | CDR-L3 | |
| 588 | Nectin-4 VH | EVQLVESGGGLVQPGGSLRLSCAASGFTFSSYNMNWVRQAPGK |
| | | GLEWVSYISSSSSTIYY |
| | | ADSVKGRFTISRDNAKNSLSLQMNSLRDEDTAVYYCARAYYYG |
| | | MDVWGQGTTVTVSS |
| 589 | Nectin-4 VL | DIQMTQSPSSVSASVGDRVTITCRASQGISGWLAWYQQKPGKAP |
| | | KFLIYAASTLQSGVPS |
| | | RFSGSGSGTDFTLTISSLQPEDFATYYCQQANSFPPTFGGGTKVEI |
| | | K |
| 590 | SLTRK6 | SYGMH |
| | CDR-H1 | |
| 591 | SLTRK6 | VIWYDGSNQYYADSVKG |
| | CDR-H2 | |
| 592 | SLTRK6 | GLTSGRYGMDV |
| | CDR-H3 | |
| 593 | SLTRK6 | RSSQSLLLSHGFNYLD |
| | CDR-L1 | |
| 594 | SLTRK6 | LGSSRAS |
| | CDR-L2 | |
| 595 | SLTRK6 | MQPLQIPWT |
| | CDR-L3 | |
| 596 | SLTRK6 VH | QVQLVESGGGVVQPGRSLRLSCAASGFTFSSYGMHWVRQAPGK |
| | | GLEWVAVIWYDGSNQYY |
| | | ADSVKGRFTISRDNSKNTLFLQMHSLRAEDTAVYYCARGLTSGR |
| | | YGMDVWGQGTTVTVSS |
| 597 | SLTRK6 VL | DIVMTQSPLSLPVTPGEPASISCRSSQSLLLSHGFNYLDWYLQKP |
| | | GQSPQLLIYLGSSRASGVPDRFSGSGSGTDFTLKISRVEAEDVGL |
| | | YYCMQPLQIPWTFGQGTKVEIK |
| 598 | CD228 | SGYWN |
| | CDR-H1 | |
| 599 | CD228 | YISDSGITYYNPSLKS |
| | CDR-H2 | |
| 600 | CD228 | RTLATYYAMDY |
| | CDR-H3 | |
| 601 | CD228 | RASQSLVHSDGNTYLH |
| | CDR-L1 | |
| 602 | CD228 | RVSNRFS |
| | CDR-L2 | |
| 603 | CD228 | SQSTHVPPT |
| | CDR-L3 | |
| 604 | CD228 VH | QVQLQESGPGLVKPSETLSLTCTVSGDSITSGYWNWIRQPPGKGL |
| | | EYIGYISDSGITYYN |
| | | PSLKSRVTISRDTSKNQYSLKLSSVTAADTAVYYCARRTLATYY |
| | | AMDYWGQGTLVTVSS |
| 605 | CD228 VL | DFVMTQSPLSLPVTLGQPASISCRASQSLVHSDGNTYLHWYQQR |
| | | PGQSPRLLIYRVSNRFSGVPDRFSGSGSGTDFTLKISRVEAEDVG |
| | | VYYCSQSTHVPPTFGQGTKLEIKR |
| 606 | CD142 (TF) | NYAMS |
| | CDR-H1 | |
| 607 | CD142 (TF) | SISGSGDYTYYTDSVKG |
| | CDR-H2 | |
| 608 | CD142 (TF) | SPWGYYLDS |
| | CDR-H3 | |
| 609 | CD142 (TF) | RASQGISSRLA |
| | CDR-L1 | |
| 610 | CD142 (TF) | AASSLQS |
| | CDR-L2 | |
| 611 | CD142 (TF) | QQYNSYPYT |
| | CDR-L3 | |
| 612 | CD142 (TF) | EVQLLESGGGLVQPGGSLRLSCAASGFTFSNYAMSWVRQAPGK |
| | VH | GLEWVSSISGSGDYTY |
| | | YTDSVKGRFTISRDNSKNTLYLQMNSLRAEDTAVYYCARSPWG |
| | | YYLDSWGQGTLVTVSS |
| 613 | CD142 (TF) | DIQMTQSPPSLSASAGDRVTITCRASQGISSRLAWYQQKPEKAPK |
| | VL | SLIYAASSLQSGVPS |
| | | RFSGSGSGTDFTLTISSLQPEDFATYYCQQYNSYPYTFGQGTKLEI |
| | | K |
| 614 | STn CDR- | DHAIH |
| | H1 | |
| 615 | STn CDR- | YFSPGNDDIKYNEKFRG |
| | H2 | |
| 616 | STn CDR- | SLSTPY |
| | H3 | |
| 617 | STn CDR-L1 | KSSQSLLNRGNHKNYLT |
| 618 | STn CDR-L2 | WASTRES |
| 619 | STn CDR-L3 | QNDYTYPYT |
| 620 | STn VH | EVQLVQSGAEVKKPGASVKVSCKASGYTFTDHAIHWVRQAPGQ |
| | | GLEWMGYFSPGNDDIKY |
| | | NEKFRGRVTMTADKSSSTAYMELRSLRSDDTAVYFCKRSLSTPY |
| | | WGQGTLVTVSS |
| 621 | STn VL | DIVMTQSPDSLAVSLGERATINCKSSQSLLNRGNHKNYLTWYQQ |
| | | KPGQPPKLLIYWAST |
| | | RESGVPDRFSGSGSGTDFTLTISSLQAEDVAVYYCQNDYTYPYTF |
| | | GQGTKVEIK |
| 622 | CD20 CDR- | SYNMH |
| | H1 | |
| 623 | CD20 CDR- | AIYPGNGDTSYNQKFKG |
| | H2 | |
| 624 | CD20 CDR- | STYYGGDWYFNV |
| | H3 | |
| 625 | CD20 CDR- | RASSSVSYIH |
| | L1 | |
| 626 | CD20 CDR- | ATSNLAS |
| | L2 | |
| 627 | CD20 CDR- | QQWTSNPPT |
| | L3 | |
| 628 | CD20 VH | QVQLQQPGAELVKPGASVKMSCKASGYTFTSYNMHWVKQTPG |
| | | RGLEWIGAIYPGNGDTSY |
| | | NQKFKGKATLTADKSSSTAYMQLSSLTSEDSAVYYCARSTYYG |
| | | GDWYFNVWGAGTTVTVSA |
| 629 | CD20 VL | QIVLSQSPAILSASPGEKVTMTCRASSSVSYIHWFQQKPGSSPKP |
| | | WIYATSNLASGVPVR |
| | | FSGSGSGTSYSLTISRVEAEDAATYYCQQWTSNPPTFGGGTKLEI |
| | | K |
| 630 | HER2 CDR- | DTYIH |
| | H1 | |
| 631 | HER2 CDR- | RIYPTNGYTRYADSVKG |
| | H2 | |
| 632 | HER2 CDR- | WGGDGFYAMDY |
| | H3 | |
| 633 | HER2 CDR- | RASQDVNTAVA |
| | L1 | |
| 634 | HER2 CDR- | SASFLYS |
| | L2 | |
| 635 | HER2 CDR- | QQHYTTPPT |
| | L3 | |
| 636 | HER2 VH | EVQLVESGGGLVQPGGSLRLSCAASGFNIKDTYIHWVRQAPGKG |
| | | LEWVARIYPTNGYTRY |
| | | ADSVKGRFTISADTSKNTAYLQMNSLRAEDTAVYYCSRWGGDG |
| | | FYAMDYWGQGTLVTVSS |
| 637 | HER2 VL | DIQMTQSPSSLSASVGDRVTITCRASQDVNTAVAWYQQKPGKAP |
| | | KLLIYSASFLYSGVPS |
| | | RFSGSRSGTDFTLTISSLQPEDFATYYCQQHYTTPPTFGQGTKVEI |
| | | K |
| 638 | CD79b | SYWIE |
| | CDR-H1 | |
| 639 | CD79b | EILPGGGDTNYNEIFKG |
| | CDR-H2 | |
| 640 | CD79b | RVPIRLDY |
| | CDR-H3 | |
| 641 | CD79b | KASQSVDYEGDSFLN |
| | CDR-L1 | |
| 642 | CD79b | AASNLES |
| | CDR-L2 | |
| 643 | CD79b | QQSNEDPLT |
| | CDR-L3 | |
| 644 | CD79b VH | EVQLVESGGGLVQPGGSLRLSCAASGYTFSSYWIEWVRQAPGK |
| | | GLEWIGEILPGGGDTNYNEIFKGRATFSADTSKNTAYLQMNSLR |
| | | AEDTAVYYCTRRVPIRLDYWGQGTLVTVSS |
| 645 | CD79b VL | DIQLTQSPSSLSASVGDRVTITCKASQSVDYEGDSFLNWYQQKP |
| | | GKAPKLLIYAASNLES |
| | | GVPSRFSGSGSGTDFTLTISSLQPEDFATYYCQQSNEDPLTFGQGT |
| | | KVEIK |
| 646 | NaPi2B | DFAMS |
| | CDR-H1 | |
| 647 | NaPi2B | TIGRVAFHTYYPDSMKG |
| | CDR-H2 | |
| 648 | NaPi2B | HRGFDVGHFDF |
| | CDR-H3 | |
| 649 | NaPi2B | RSSETLVHSSGNTYLE |
| | CDR-L1 | |
| 650 | NaPi2B | RVSNRFS |
| | CDR-L2 | |
| 651 | NaPi2B | FQGSFNPLT |
| | CDR-L3 | |
| 652 | NaPi2B VH | EVQLVESGGGLVQPGGSLRLSCAASGFSFSDFAMSWVRQAPGK |
| | | GLEWVATIGRVAFHTYY |
| | | PDSMKGRFTISRDNSKNTLYLQMNSLRAEDTAVYYCARHRGFD |
| | | VGHFDFWGQGTLVTVSS |
| 653 | NaPi2B VL | DIQMTQSPSSLSASVGDRVTITCRSSETLVHSSGNTYLEWYQQKP |
| | | GKAPKLLIYRVSNRF |
| | | SGVPSRFSGSGSGTDFTLTISSLQPEDFATYYCFQGSFNPLTFGQG |
| | | TKVEIK |
| 654 | Muc16 | NDYAWN |
| | CDR-H1 | |
| 655 | Muc16 | YISYSGYTTYNPSLKS |
| | CDR-H2 | |
| 656 | Muc16 | WTSGLDY |
| | CDR-H3 | |
| 657 | Muc16 | KASDLIHNWLA |
| | CDR-L1 | |
| 658 | Muc16 | GATSLET |
| | CDR-L2 | |
| 659 | Muc16 | QQYWTTPFT |
| | CDR-L3 | |
| 660 | Muc16 VH | EVQLVESGGGLVQPGGSLRLSCAASGYSITNDYAWNWVRQAPG |
| | | KGLEWVGYISYSGYTTY |
| | | NPSLKSRFTISRDTSKNTLYLQMNSLRAEDTAVYYCARWTSGLD |
| | | YWGQGTLVTVSS |
| 661 | Muc16 VL | DIQMTQSPSSLSASVGDRVTITCKASDLIHNWLAWYQQKPGKAP |
| | | KLLIYGATSLETGVPSRFSGSGSGTDFTLTISSLQPEDFATYYCQQ |
| | | YWTTPFTFGQGTKVEIK |
| 662 | STEAP1 | SDYAWN |
| | CDR-H1 | |
| 663 | STEAP1 | YISNSGSTSYNPSLKS |
| | CDR-H2 | |
| 664 | STEAP1 | ERNYDYDDYYYAMDY |
| | CDR-H3 | |
| 665 | STEAP1 | KSSQSLLYRSNQKNYLA |
| | CDR-L1 | |
| 666 | STEAP1 | WASTRES |
| | CDR-L2 | |
| 667 | STEAP1 | QQYYNYPRT |
| | CDR-L3 | |
| 668 | STEAP1 VH | EVQLVESGGGLVQPGGSLRLSCAVSGYSITSDYAWNWVRQAPG |
| | | KGLEWVGYISNSGSTSYNPSLKSRFTISRDTSKNTLYLQMNSLRA |
| | | EDTAVYYCARERNYDYDDYYYAMDYWGQGTLVTVSS |
| 669 | STEAP1 VL | DIQMTQSPSSLSASVGDRVTITCKSSQSLLYRSNQKNYLAWYQQ |
| | | KPGKAPKLLIYWASTRESGVPSRFSGSGSGTDFTLTISSLQPEDFA |
| | | TYYCQQYYNYPRTFGQGTKVEIK |
| 670 | BCMA | NYWMH |
| | CDR-H1 | |
| 671 | BCMA | ATYRGHSDTYYNQKFKG |
| | CDR-H2 | |
| 672 | BCMA | GAIYDGYDVLDN |
| | CDR-H3 | |
| 673 | BCMA | SASQDISNYLN |
| | CDR-L1 | |
| 674 | BCMA | YTSNLHS |
| | CDR-L2 | |
| 675 | BCMA | QQYRKLPWT |
| | CDR-L3 | |
| 676 | BCMA VH | QVQLVQSGAEVKKPGSSVKVSCKASGGTFSNYWMHWVRQAPG |
| | | QGLEWMGATYRGHSDTYYNQKFKGRVTITADKSTSTAYMELSS |
| | | LRSEDTAVYYCARGAIYDGYDVLDNWGQGTLVTVSS |
| 677 | BCMA VL | DIQMTQSPSSLSASVGDRVTITCSASQDISNYLNWYQQKPGKAP |
| | | KLLIYYTSNLHSGVPSRFSGSGSGTDFTLTISSLQPEDFATYYCQQ |
| | | YRKLPWTFGQGTKLEIK |
| 678 | c-Met CDR- | AYTMH |
| | H1 | |
| 679 | c-Met CDR- | WIKPNNGLANYAQKFQG |
| | H2 | |
| 680 | c-Met CDR- | SEITTEFDY |
| | H3 | |
| 681 | c-Met CDR- | KSSESVDSYANSFLH |
| | L1 | |
| 682 | c-Met CDR- | RASTRES |
| | L2 | |
| 683 | c-Met CDR- | QQSKEDPLT |
| | L3 | |
| 684 | c-Met VH | QVQLVQSGAEVKKPGASVKVSCKASGYIFTAYTMHWVRQAPG |
| | | QGLEWMGWIKPNNGLAN |
| | | YAQKFQGRVTMTRDTSISTAYMELSRLRSDDTAVYYCARSEITT |
| | | EFDYWGQGTLVTVSS |
| 685 | c-Met VL | DIVMTQSPDSLAVSLGERATINCKSSESVDSYANSFLHWYQQKP |
| | | GQPPKLLIYRASTRE |
| | | SGVPDRFSGSGSGTDFTLTISSLQAEDVAVYYCQQSKEDPLTFGG |
| | | GTKVEIK |
| 686 | EGFR CDR- | SDFAWN |
| | H1 | |
| 687 | EGFR CDR- | YISYSGNTRYQPSLKS |
| | H2 | |
| 688 | EGFR CDR- | AGRGFPY |
| | H3 | |
| 689 | EGFR CDR- | HSSQDINSNIG |
| | L1 | |
| 690 | EGFR CDR- | HGTNLDD |
| | L2 | |
| 691 | EGFR CDR- | VQYAQFPWT |
| | L3 | |
| 692 | EGFR VH | QVQLQESGPGLVKPSQTLSLTCTVSGYSISSDFAWNWIRQPPGKG |
| | | LEWMGYISYSGNTRY |
| | | QPSLKSRITISRDTSKNQFFLKLNSVTAADTATYYCVTAGRGFPY |
| | | WGQGTLVTVSS |
| 693 | EGFR VL | DIQMTQSPSSMSVSVGDRVTITCHSSQDINSNIGWLQQKPGKSFK |
| | | GLIYHGTNLDDGVPS |
| | | RFSGSGSGTDYTLTISSLQPEDFATYYCVQYAQFPWTFGGGTKLE |
| | | IK |
| 694 | SLAMF7 | DYYMA |
| | CDR-H1 | |
| 695 | SLAMF7 | SINYDGSSTYYVDSVKG |
| | CDR-H2 | |
| 696 | SLAMF7 | DRGYYFDY |
| | CDR-H3 | |
| 697 | SLAMF7 | RSSQSLVHSNGNTYLH |
| | CDR-L1 | |
| 698 | SLAMF7 | KVSNRFS |
| | CDR-L2 | |
| 699 | SLAMF7 | SQSTHVPPFT |
| | CDR-L3 | |
| 700 | SLAMF7 | EVQLVESGGGLVQPGGSLRLSCAASGFTFSDYYMAWVRQAPGK |
| | VH | GLEWVASINYDGSSTY |
| | | YVDSVKGRFTISRDNAKNSLYLQMNSLRAEDTAVYYCARDRGY |
| | | YFDYWGQGTTVTVSS |
| 701 | SLAMF7 VL | DVVMTQTPLSLSVTPGQPASISCRSSQSLVHSNGNTYLHWYLQK |
| | | PGQSPQLLIYKVSNRF |
| | | SGVPDRFSGSGSGTDFTLKISRVEAEDVGVYFCSQSTHVPPFTFG |
| | | GGTKVEIK |
| 702 | SLITRK6 | SYGMH |
| | CDR-H1 | |
| 703 | SLITRK6 | VIWYDGSNQYYADSVKG |
| | CDR-H2 | |
| 704 | SLITRK6 | GLTSGRYGMDV |
| | CDR-H3 | |
| 705 | SLITRK6 | RSSQSLLLSHGFNYLD |
| | CDR-L1 | |
| 706 | SLITRK6 | LGSSRAS |
| | CDR-L2 | |
| 707 | SLITRK6 | MQPLQIPWT |
| | CDR-L3 | |
| 708 | SLITRK6 | QVQLVESGGGVVQPGRSLRLSCAASGFTFSSYGMHWVRQAPGK |
| | VH | GLEWVAVIWYDGSNQYY |
| | | ADSVKGRFTISRDNSKNTLFLQMHSLRAEDTAVYYCARGLTSGR |
| | | YGMDVWGQGTTVTVSS |
| 709 | SLITRK6 | DIVMTQSPLSLPVTPGEPASISCRSSQSLLLSHGFNYLDWYLQKP |
| | VL | GQSPQLLIYLGSSRA |
| | | SGVPDRFSGSGSGTDFTLKISRVEAEDVGLYYCMQPLQIPWTFG |
| | | QGTKVEIK |
| 710 | C4.4a CDR- | NAWMS |
| | H1 | |
| 711 | C4.4a CDR- | YISSSGSTIYYADSVKG |
| | H2 | |
| 712 | C4.4a CDR- | EGLWAFDY |
| | H3 | |
| 713 | C4.4a CDR- | TGSSSNIGAGYVVH |
| | L1 | |
| 714 | C4.4a CDR- | DNNKRPS |
| | L2 | |
| 715 | C4.4a CDR- | AAWDDRLNGPV |
| | L3 | |
| 716 | C4.4a VH | EVQLLESGGGLVQPGGSLRLSCAASGFTFSNAWMSWVRQAPGK |
| | | GLEWVSYISSSGSTIYY |
| | | ADSVKGRFTISRDNSKNTLYLQMNSLRAEDTAVYYCAREGLWA |
| | | FDYWGQGTLVTVSS |
| 717 | C4.4a VL | ESVLTQPPSVSGAPGQRVTISCTGSSSNIGAGYVVHWYQQLPGT |
| | | APKLLIYDNNKRPSGV |
| | | PDRFSGSKSGTSASLAISGLRSEDEADYYCAAWDDRLNGPVFGG |
| | | GTKLTVL |
| 718 | GCC CDR- | GYYWS |
| | H1 | |
| 719 | GCC CDR- | EINHRGNTNDNPSLKS |
| | H2 | |
| 720 | GCC CDR- | ERGYTYGNFDH |
| | H3 | |
| 721 | GCC CDR- | RASQSVSRNLA |
| | L1 | |
| 722 | GCC CDR- | GASTRAT |
| | L2 | |
| 723 | GCC CDR- | QQYKTWPRT |
| | L3 | |
| 724 | GCC VH | QVQLQQWGAGLLKPSETLSLTCAVFGGSFSGYYWSWIRQPPGK |
| | | GLEWIGEINHRGNTNDN |
| | | PSLKSRVTISVDTSKNQFALKLSSVTAADTAVYYCARERGYTYG |
| | | NFDHWGQGTLVTVSS |
| 725 | GCC VL | EIVMTQSPATLSVSPGERATLSCRASQSVSRNLAWYQQKPGQAP |
| | | RLLIYGASTRATGIP |
| | | ARFSGSGSGTEFTLTIGSLQSEDFAVYYCQQYKTWPRTFGQGTN |
| | | VEIK |
| 726 | Axl CDR-H1 | SYAMN |
| 727 | Axl CDR-H2 | TTSGSGASTYYADSVKG |
| 728 | Axl CDR-H3 | IWIAFDI |
| 729 | Axl CDR-L1 | RASQSVSSSYLA |
| 730 | Axl CDR-L2 | GASSRAT |
| 731 | Axl CDR-L3 | QQYGSSPYT |
| 732 | Axl VH | EVQLLESGGGLVQPGGSLRLSCAASGFTFSSYAMNWVRQAPGK |
| | | GLEWVSTTSGSGASTYY |
| | | ADSVKGRFTISRDNSKNTLYLQMNSLRAEDTAVYYCAKIWIAFD |
| | | IWGQGTMVTVSS |
| 733 | Axl VL | EIVLTQSPGTLSLSPGERATLSCRASQSVSSSYLAWYQQKPGQAP |
| | | RLLIYGASSRATGIP |
| | | DRFSGSGSGTDFTLTISRLEPEDFAVYYCQQYGSSPYTFGQGTKL |
| | | EIK |
| 734 | gpNMB | SFNYYWS |
| | CDR-H1 | |
| 735 | gpNMB | YIYYSGSTYSNPSLKS |
| | CDR-H2 | |
| 736 | gpNMB | GYNWNYFDY |
| | CDR-H3 | |
| 737 | gpNMB | RASQSVDNNLV |
| | CDR-L1 | |
| 738 | gpNMB | GASTRAT |
| | CDR-L2 | |
| 739 | gpNMB | QQYNNWPPWT |
| | CDR-L3 | |
| 740 | gpNMB VH | QVQLQESGPGLVKPSQTLSLTCTVSGGSISSFNYYWSWIRHHPGK |
| | | GLEWIGYIYYSGSTY |
| | | SNPSLKSRVTISVDTSKNQFSLTLSSVTAADTAVYYCARGYNWN |
| | | YFDYWGQGTLVTVSS |
| 741 | gpNMB VL | EIVMTQSPATLSVSPGERATLSCRASQSVDNNLVWYQQKPGQAP |
| | | RLLIYGASTRATGIPA |
| | | RFSGSGSGTEFTLTISSLQSEDFAVYYCQQYNNWPPWTFGQGTK |
| | | VEIK |
| 742 | Prolactin | TYWMH |
| | receptor | |
| | CDR-H1 | |
| 743 | Prolactin | EIDPSDSYSNYNQKFKD |
| | receptor | |
| | CDR-H2 | |
| 744 | Prolactin | NGGLGPAWFSY |
| | receptor | |
| | CDR-H3 | |
| 745 | Prolactin | KASQYVGTAVA |
| | receptor | |
| | CDR-L1 | |
| 746 | Prolactin | SASNRYT |
| | receptor | |
| | CDR-L2 | |
| 747 | Prolactin | QQYSSYPWT |
| | receptor | |
| | CDR-L3 | |
| 748 | Prolactin | EVQLVQSGAEVKKPGSSVKVSCKASGYTFTTYWMHWVRQAPG |
| | receptor VH | QGLEWIGEIDPSDSYSNY |
| | | NQKFKDRATLTVDKSTSTAYMELSSLRSEDTAVYYCARNGGLG |
| | | PAWFSYWGQGTLVTVSS |
| 749 | Prolactin | DIQMTQSPSSVSASVGDRVTITCKASQYVGTAVAWYQQKPGKSP |
| | receptor VL | KLLIYSASNRYTGVPS |
| | | RFSDSGSGTDFTLTISSLQPEDFATYFCQQYSSYPWTFGGGTKVEI |
| | | K |
| 750 | FGFR2 | SYAMS |
| | CDR-H1 | |
| 751 | FGFR2 | AISGSGTSTYYADSVKG |
| | CDR-H2 | |
| 752 | FGFR2 | VRYNWNHGDWFDP |
| | CDR-H3 | |
| 753 | FGFR2 | SGSSSNIGNNYVS |
| | CDR-L1 | |
| 754 | FGFR2 | ENYNRPA |
| | CDR-L2 | |
| 755 | FGFR2 | SSWDDSLNYWV |
| | CDR-L3 | |
| 756 | FGFR2 VH | EVQLLESGGGLVQPGGSLRLSCAASGFTFSSYAMSWVRQAPGK |
| | | GLEWVSAISGSGTSTYYADSVKGRFTISRDNSKNTLYLQMNSLR |
| | | AEDTAVYYCARVRYNWNHGDWFDPWGQGTLVTVSS |
| 757 | FGFR2 VL | QSVLTQPPSASGTPGQRVTISCSGSSSNIGNNYVSWYQQLPGTAP |
| | | KLLIYENYNRPAGVP |
| | | DRFSGSKSGTSASLAISGLRSEDEADYYCSSWDDSLNYWVFGGG |
| | | TKLTVL |
| 758 | CDCP1 | SYGMS |
| | CDR-H1 | |
| 759 | CDCP1 | TISSGGSYKYYVDSVKG |
| | CDR-H2 | |
| 760 | CDCP1 | HPDYDGVWFAY |
| | CDR-H3 | |
| 761 | CDCP1 | SVSSSVFYVH |
| | CDR-L1 | |
| 762 | CDCP1 | DTSKLAS |
| | CDR-L2 | |
| 763 | CDCP1 | QQWNSNPPT |
| | CDR-L3 | |
| 764 | CDCP1 VH | EVQLVESGGGLVQPGGSLRLSCAASGFTFNSYGMSWVRQAPGK |
| | | GLEWVATISSGGSYKYY |
| | | VDSVKGRFTISRDNAKNSLYLQMNSLRAEDTAVYYCARHPDYD |
| | | GVWFAYWGQGTLVTVSS |
| 765 | CDCP1 VL | DIQMTQSPSSLSASVGDRVTITCSVSSSVFYVHWYQQKPGKAPK |
| | | LLIYDTSKLASSGVPS |
| | | RFSGSGSGTDFTFTISSLQPEDIATYYCQQWNSNPPTFGGGTKVEI |
| | | K |
| 766 | CDCP1 | SYGMS |
| | CDR-H1 | |
| 767 | CDCP1 | TISSGGSYTYYPDSVKG |
| | CDR-H2 | |
| 768 | CDCP1 | HPDYDGVWFAY |
| | CDR-H3 | |
| 769 | CDCP1 | SVSSSVFYVH |
| | CDR-L1 | |
| 770 | CDCP1 | DTSKLAS |
| | CDR-L2 | |
| 771 | CDCP1 | QQWNSNPPT |
| | CDR-L3 | |
| 772 | CDCP1 VH | EVQLVESGGDLVKPGGSLKLSCAASGFTFNSYGMSWVRQTPDK |
| | | RLEWVATISSGGSYTYY |
| | | PDSVKGRFTISRDNAKNTLYLQMSSLKSEDTAMYYCARHPDYD |
| | | GVWFAYWGQGTLVTVSA |
| 773 | CDCP1 VL | QIVLTQSPAIMASPGEKVTMTCSVSSSVFYVHWYQQKSGTSPKR |
| | | WIYDTSKLASGVPARF |
| | | SGSGSGTSYSLTISSMEAEDAATYYCQQWNSNPPTFGGGTKLEIK |
| 774 | CDCP1 | SYYMH |
| | CDR-H1 | |
| 775 | CDCP1 | IINPSGGSTSYAQKFQG |
| | CDR-H2 | |
| 776 | CDCP1 | DGVLRYFDWLLDYYYY |
| | CDR-H3 | |
| 777 | CDCP1 | RASQSVGSYLA |
| | CDR-L1 | |
| 778 | CDCP1 | DASNRAT |
| | CDR-L2 | |
| 779 | CDCP1 | QQRANVFT |
| | CDR-L3 | |
| 780 | CDCP1 VH | EVQLVQSGAEVKKPGASVKVSCKASGYTFTSYYMHWVRQAPG |
| | | QGLEWMGIINPSGGSTSY |
| | | AQKFQGRVTMTRDTSTSTVYMELSSLRSEDTAVYYCARDGVLR |
| | | YFDWLLDYYYYMDVWGKG |
| | | TTVTVSS |
| 781 | CDCP1 VL | EIVLTQSPATLSLSPGERATLSCRASQSVGSYLAWYQQRPGQAPR |
| | | LLIYDASNRATGIPA |
| | | RFSGSGSGTDFTLTISSLEPEDFAVYYCQQRANVFTFGQGTKVEI |
| | | K |
| 782 | CDCP1 | SYYMH |
| | CDR-H1 | |
| 783 | CDCP1 | IINPSGGSTSYAQKFQG |
| | CDR-H2 | |
| 784 | CDCP1 | DAELRHFDHLLDYHYYMDV |
| | CDR-H3 | |
| 785 | CDCP1 | RASQSVGSYLA |
| | CDR-L1 | |
| 786 | CDCP1 | DASNRAT |
| | CDR-L2 | |
| 787 | CDCP1 | QQRAQEFT |
| | CDR-L3 | |
| 788 | CDCP1 VH | EVQLVQSGAEVKKPGASVKVSCKASGYTFTSYYMHWVRQAPG |
| | | QGLEWMGIINPSGGSTSYAQKFQGRVTMTRDTSTSTVYMELSSL |
| | | RSEDTAVYYCARDAELRHFDHLLDYHYYMDVWGQGTTVTVSS |
| 789 | CDCP1 VL | EIVMTQSPATLSLSPGERATLSCRASQSVGSYLAWYQQKPGQAP |
| | | RLLIYDASNRATGIPA |
| | | RFSGSGSGTDFTLTISSLQPEDFAVYYCQQRAQEFTFGQGTKVEI |
| | | K |
| 790 | ASCT2 VH | QVQLVQSGSELKKPGAPVKVSCKASGYTFSTFGMSWVRQAPGQ |
| | | GLKWMGWIHTYAGVPIYGDDFKGRFVFSLDTSVSTAYLQISSLK |
| | | AEDTAVYFCARRSDNYRYFFDYWGQGTTVTVSS |
| 791 | ASCT2 VL | DIQMTQSPSSLSASLGDRVTITCRASQDIRNYLNWYQQKPGKAP |
| | | KLLIYYTSRLHSGVPSRFSGSGSGTDYTLTISSLQPEDFATYFCQQ |
| | | GHTLPPTFGQGTKLEIK |
| 792 | ASCT2 VH | QIQLVQSGPELKKPGAPVKISCKASGYTFTTFGMSWVKQAPGQG |
| | | LKWMGWIHTYAGVPIYGDDFKGRFVFSLDTSVSTAYLQISSVKA |
| | | EDTATYFCARRSDNYRYFFDYWGQGTTLTVSS |
| 793 | ASCT2 VL | DIQMTQSPSSLSASLGDRVTITCRASQDIRNYLNWYQQKPGKAP |
| | | KLLIYYTSRLHSGVPS |
| | | RFSGSGSGTDYTLTISSLQPEDFATYFCQQGHTLPPTFGQGTKLEI |
| | | K |
| 794 | ASCT2 | NYYMA |
| | CDR-H1 | |
| 795 | ASCT2 | SITKGGGNTYYRDSVKG |
| | CDR-H2 | |
| 796 | ASCT2 | QVTIAAVSTSYFDS |
| | CDR-H3 | |
| 797 | ASCT2 | KTNQKVDYYGNSYVY |
| | CDR-L1 | |
| 798 | ASCT2 | LASNLAS |
| | CDR-L2 | |
| 799 | ASCT2 | QQSRNLPYT |
| | CDR-L3 | |
| 800 | ASCT2 VH | EVQLVESGGGLVQSGRSIRLSCAASGFSFSNYYMAWVRQAPSKG |
| | | LEWVASITKGGGNTYYRDSVKGRFTFSRDNAKSTLYLQMDSLR |
| | | SEDTATYYCARQVTIAAVSTSYFDSWGQGVMVTVSS |
| 801 | ASCT2 VL | DIVLTQSPALAVSLGQRATISCKTNQKVDYYGNSYVYWYQQKP |
| | | GQQPKLLIYLASNLASGIPARFSGRGSGTDFTLTIDPVEADDTAT |
| | | YYCQQSRNLPYTFGAGTKLELK |
| 802 | CD123 | DYYMK |
| | CDR-H1 | |
| 803 | CD123 | DIIPSNGATFYNQKFKG |
| | CDR-H2 | |
| 804 | CD123 | SHLLRASWFAY |
| | CDR-H3 | |
| 805 | CD123 | KSSQSLLNSGNQKNYLT |
| | CDR-L1 | |
| 806 | CD123 | WASTRES |
| | CDR-L2 | |
| 807 | CD123 | QNDYSYPYT |
| | CDR-L3 | |
| 808 | CD123 VH | QVQLVQSGAEVKKPGASVKMSCKASGYTFTDYYMKWVKQAP |
| | | GQGLEWIGDIIPSNGATFYNQKFKGKATLTVDRSISTAYMHLNR |
| | | LRSDDTAVYYCTRSHLLRASWFAYWGQGTLVTVSS |
| 809 | CD123 VL | DFVMTQSPDSLAVSLGERATINCKSSQSLLNSGNQKNYLTWYLQ |
| | | KPGQPPKLLIYWASTRESGVPDRFSGSGSGTDFTLTISSLQAEDV |
| | | AVYYCQNDYSYPYTFGQGTKLEIK |
| 810 | GPC3 CDR- | DYEMH |
| | H1 | |
| 811 | GPC3 CDR- | WIGGIDPETGGTAYNQKFKG |
| | H2 | |
| 812 | GPC3 CDR- | YYSFAY |
| | H3 | |
| 813 | GPC3 CDR- | RSSQSIVHSNGNTYLQ |
| | L1 | |
| 814 | GPC3 CDR- | KVSNRFS |
| | L2 | |
| 815 | GPC3 CDR- | FQVSHVPYT |
| | L3 | |
| 816 | GPC3 VH | EVQLVQSGAEVKKPGATVKISCKVSGYTFTDYEMHWVQQAPG |
| | | KGLEWMGGIDPETGGTAYNQKFKGRVTLTADKSTDTAYMELSS |
| | | LRSEDTAVYYCGRYYSFAYWGQGTLVTVSS |
| 817 | GPC3 VL | DVVMTQSPLSLPVTLGQPASISCRSSQSIVHSNANTYLQWFQQRP |
| | | GQSPRLLIYKVSNRFSGVPDRFSGSGSGTDFTLKISRVEAEDVGV |
| | | YYCFQVSHVPYTFGQGTKLEIK |
| 818 | B6A CDR- | DYNVN |
| | H1 | |
| 819 | B6A CDR- | VINPKYGTTRYNQKFKG |
| | H2 | |
| 820 | B6A CDR- | GLNAWDY |
| | H3 | |
| 821 | B6A CDR- | GASENIYGALN |
| | L1 | |
| 822 | B6A CDR- | GATNLED |
| | L2 | |
| 823 | B6A CDR- | QNVLTTPYT |
| | L3 | |
| 824 | B6A VH | QFQLVQSGAEVKKPGASVKVSCKASGYSFTDYNVNWVRQAPG |
| | | QGLEWIGVINPKYGTTRYNQKFKGRATLTVDKSTSTAYMELSSL |
| | | RSEDTAVYYCTRGLNAWDYWGQGTLVTVSS |
| 825 | B6A VL | DIQMTQSPSSLSASVGDRVTITCGASENIYGALNWYQQKPGKAP |
| | | KLLIYGATNLEDGVPSRFSGSGSGRDYTFTISSLQPEDIATYYCQN |
| | | VLTTPYTFGQGTKLEIK |
| 826 | B6A CDR- | GYFMN |
| | H1 | |
| 827 | B6A CDR- | linpyngdsfynqkfkg |
| | H2 | |
| 828 | B6A CDR- | glrrdfdy |
| | H3 | |
| 829 | B6A CDR- | kssqslldsdgktyln |
| | L1 | |
| 830 | B6A CDR- | lvselds |
| | L2 | |
| 831 | B6A CDR- | wqgthfprt |
| | L3 | |
| 832 | B6A VH | QVQLVQSGAEVKKPGASVKVSCKASGYSFSGYFMNWVRQAPG |
| | | QGLEWMGLINPYNGDSFYNQKFKGRVTMTRQTSTSTVYMELSS |
| | | LRSEDTAVYYCVRGLRRDFDYWGQGTLVTVSS |
| 833 | B6A VL | DVVMTQSPLSLPVTLGQPASISCKSSQSLLDSDGKTYLNWLFQRP |
| | | GQSPRRLIYLVSELDSGVPDRFSGSGSGTDFTLKISRVEAEDVGV |
| | | YYCWQGTHFPRTFGGGTKLEIK |
| 834 | PD-L1 CDR- | TAAIS |
| | H1 | |
| 835 | PD-L1 CDR- | GIIPIFGKAHYAQKFQG |
| | H2 | |
| 836 | PD-L1 CDR- | KFHFVSGSPFGMDV |
| | H3 | |
| 837 | PD-L1 CDR- | RASQSVSSYLA |
| | L1 | |
| 838 | PD-L1 CDR- | DASNRAT |
| | L2 | |
| 839 | PD-L1 CDR- | QQRSNWPT |
| | L3 | |
| 840 | PD-L1 VH | QVQLVQSGAEVKKPGSSVKVSCKTSGDTFSTAAISWVRQAPGQ |
| | | GLEWMGGIIPIFGKAHYAQKFQGRVTITADESTSTAYMELSSLRS |
| | | EDTAVYFCARKFHFVSGSPFGMDVWGQGTTVTVSS |
| 841 | PD-L1 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPTFGQGTKVEIK |
| 842 | TIGIT CDR- | GTFSSYAIS |
| | H1 | |
| 843 | TIGIT CDR- | SIIPIFGTANYAQKFQG |
| | H2 | |
| 844 | TIGIT CDR- | ARGPSEVGAILGYVWFDP |
| | H3 | |
| 845 | TIGIT CDR- | RSSQSLLHSNGYNYLD |
| | L1 | |
| 846 | TIGIT CDR- | LGSNRAS |
| | L2 | |
| 847 | TIGIT CDR- | MQARRIPIT |
| | L3 | |
| 848 | TIGIT VH | QVQLVQSGAEVKKPGSSVKVSCKASGGTFSSYAISWVRQAPGQ |
| | | GLEWMGSIIPIFGTANYAQKFQGRVTITADESTSTAYMELSSLRS |
| | | EDTAVYYCARGPSEVGAILGYVWFDPWGQGTLVTVSS |
| 849 | TIGIT VL | DIVMTQSPLSLPVTPGEPASISCRSSQSLLHSNGYNYLDWYLQKP |
| | | GQSPQLLIYLGSNRASGVPDRFSGSGSGTDFTLKISRVEAEDVGV |
| | | YYCMQARRIPITFGGGTKVEIK |
| 850 | STN CDR- | GYTFTDHAIHWV |
| | H1 | |
| 851 | STN CDR- | FSPGNDDIKY |
| | H2 | |
| 852 | STN CDR- | KRSLSTPY |
| | H3 | |
| 853 | STN CDR- | QSLLNRGNHKNY |
| | L1 | |
| 854 | STN CDR- | WASTRES |
| | L2 | |
| 855 | STN CDR- | QNDYTYPYT |
| | L3 | |
| 856 | STN VH | EVQLVQSGAEVKKPGASVKVSCKASGYTFTDHAIHWVRQAPGQ |
| | | GLEWMGYFSPGNDDIKYNEKFRGRVTMTADKSSSTAYMELRSL |
| | | RSDDTAVYFCKRSLSTPYWGQGTLVTVSS |
| 857 | STN VL | DIVMTQSPDSLAVSLGERATINCKSSQSLLNRGNHKNYLTWYQQ |
| | | KPGQPPKLLIYWASTRESGVPDRFSGSGSGTDFTLTISSLQAEDV |
| | | AVYYCQNDYTYPYTFGQGTKVEIK |
| 858 | CD33 CDR- | NYDIN |
| | H1 | |
| 859 | CD33 CDR- | WIYPGDGSTKYNEKFKA |
| | H2 | |
| 860 | CD33 CDR- | GYEDAMDY |
| | H3 | |
| 861 | CD33 CDR- | KASQDINSYLS |
| | L1 | |
| 862 | CD33 CDR- | RANRLVD |
| | L2 | |
| 863 | CD33 CDR- | LQYDEFPLT |
| | L3 | |
| 864 | CD33 VH | QVQLVQSGAE VKKPGASVKV SCKASGYTFT NYDINWVRQA |
| | | PGQGLEWIGW IYPGDGSTKY NEKFKAKATL TADTSTSTAY |
| | | MELRSLRSDD TAVYYCASGY EDAMDYWGQG TTVTVSS |
| 865 | CD33 VL | DIQMTQSPS SLSASVGDRVT |
| | | INCKASQDINSYLSWFQQKPGKAPKTL IYRANRLVDGVPS |
| | | RFSGSGSGQDYTLT ISSLQPEDFATYYCLQYDEFPLTFGGGTKVE |
| 866 | NTBA CDR- | NYGMN |
| | H1 | |
| 867 | NTBA CDR- | WINTYSGEPRYADDFKG |
| | H2 | |
| 868 | NTBA CDR- | DYGRWYFDV |
| | H3 | |
| 869 | NTBA CDR- | RASSSVSHMH |
| | L1 | |
| 870 | NTBA CDR- | ATSNLAS |
| | L2 | |
| 871 | NTBA CDR- | QQWSSTPRT |
| | L3 | |
| 872 | NTBA VH | QIQLVQSGSELKKPGASVKVSCKASGYTFTNYGMNWVRQAPGQ |
| | | DLKWMGWINTYSGEPRYADDFKGRFVFSLDKSVNTAYLQISSL |
| | | KAEDTAVYYCARDYGRWYFDVWGQGTTVTVSS |
| 873 | NTBA VL | QIVLSQSPATLSLSPGERATMSCRASSSVSHMHWYQQKPGQAPR |
| | | PWIYATSNLASGVPARFSGSGSGTDYTLTISSLEPEDFAVYYCQQ |
| | | WSSTPRTFGGGTKVEIK |
| 874 | BCMA | DYYIH |
| | CDR-H1 | |
| 875 | BCMA | YINPNSGYTNYAQKFQG |
| | CDR-H2 | |
| 876 | BCMA | YMWERVTGFFDF |
| | CDR-H3 | |
| 877 | BCMA | LASEDISDDLA |
| | CDR-L1 | |
| 878 | BCMA | TTSSLQS |
| | CDR-L2 | |
| 879 | BCMA | QQTYKFPPT |
| | CDR-L3 | |
| 880 | BCMA VH | QVQLVQSGAEVKKPGASVKLSCKASGYTFTDYYIHWVRQAPGQ |
| | | GLEWIGYINPNSGYTNYAQKFQGRATMTADKSINTAYVELSRLR |
| | | SDDTAVYFCTRYMWERVTGFFDFWGQGTMVTVSS |
| 881 | BCMA VL | DIQMTQSPSSVSASVGDRVTITCLASEDISDDLAWYQQKPGKAP |
| | | KVLVYTTSSLQSGVPSRFSGSGSGTDFTLTISSLQPEDFATYFCQQ |
| | | TYKFPPTFGGGTKVEIK |
| 882 | TF CDR-H1 | GFTFSNYA |
| 883 | TF CDR-H2 | ISGSGDYT |
| 884 | TF CDR-H3 | ARSPWGYYLDS |
| 885 | TF CDR-L1 | QGISSR |
| 886 | TF CDR-L2 | AAS |
| 887 | TF CDR-L3 | QQYNSYPYT |
| 888 | TF VH | EVQLLESGGGLVQPGGSLRLSCAASGFTFSNYAMSWVRQAPGK |
| | | GLEWVSSISGSGDYTYYTDSVKGRFTISRDNSKNTLYLQMNSLR |
| | | AEDTAVYYCARSPWGYYLDSWGQGTLVTVSS |
| 889 | TF VL | DIQMTQSPPSLSASAGDRVTITCRASQGISSRLAWYQQKPEKAPK |
| | | SLIYAASSLQSGVPSRFSGSGSGTDFTLTISSLQPEDFATYYCQQY |
| | | NSYPYTFGQGTKLEIK |
| 890 | Anti-PD-L1 | QVQLVQSGAEVKKPGSSVKVSCKTSGDTFSTAAISWVRQAPGQ |
| | HC | GLEWMGGIIPIFGKAHYAQKFQGRVTITADESTSTAYMELSSLRS |
| | | EDTAVYFCARKFHFVSGSPFGMDVWGQGTTVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 891 | Anti-PD-L1 | QVQLVQSGAEVKKPGSSVKVSCKTSGDTFSTAAISWVRQAPGQ |
| | HC v2 | GLEWMGGIIPIFGKAHYAQKFQGRVTITADESTSTAYMELSSLRS |
| | | EDTAVYFCARKFHFVSGSPFGMDVWGQGTTVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPG |
| 892 | Anti-PD-L1 | QVQLVQSGAEVKKPGSSVKVSCKTSGDTFSTAAISWVRQAPGQ |
| | LALA HC | GLEWMGGIIPIFGKAHYAQKFQGRVTITADESTSTAYMELSSLRS |
| | | EDTAVYFCARKFHFVSGSPFGMDVWGQGTTVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 893 | Anti-PD-L1 | QVQLVQSGAEVKKPGSSVKVSCKTSGDTFSTAAISWVRQAPGQ |
| | LALA HC | GLEWMGGIIPIFGKAHYAQKFQGRVTITADESTSTAYMELSSLRS |
| | v2 | EDTAVYFCARKFHFVSGSPFGMDVWGQGTTVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPG |
| 894 | Anti-PD-L1 | EIVLTQSPATLSLSPGERATLSCRASQSVSSYLAWYQQKPGQAPR |
| | LC | LLIYDASNRATGIPARFSGSGSGTDFTLTISSLEPEDFAVYYCQQR |
| | | SNWPTFGQGTKVEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| 895 | Anti-PD-L1 | QVQLVQSGAEVKKPGSSVKVSCKTSGDTFSTAAISWVRQAPGQ |
| | (engineered | GLEWMGGIIPIFGKAHYAQKFQGRVTITADESTSTAYMELSSLRS |
| | cysteines) | EDTAVYFCARKFHFVSGSPFGMDVWGQGTTVTVSSASTKGPSV |
| | LALA HC | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPEAAGGPCVFLFPPKPKDTLMISRTPEV |
| | | TCVVVDVSHEDPEVKFNWYVDGVEVHNAKTKPREEQYNSTYR |
| | | VVSVLTVLHQDWLNGKEYKCKVSNKALPAPIEKTISKAKGQPRE |
| | | PQVYTLPPSRDELTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPGK |
| 896 | Anti-PD-L1 | QVQLVQSGAEVKKPGSSVKVSCKTSGDTFSTAAISWVRQAPGQ |
| | (engineered | GLEWMGGIIPIFGKAHYAQKFQGRVTITADESTSTAYMELSSLRS |
| | cysteines) | EDTAVYFCARKFHFVSGSPFGMDVWGQGTTVTVSSASTKGPSV |
| | LALA HC | FPLAPSSKSTSGGTAALGCLVKDYFPEPVTVSWNSGALTSGVHT |
| | v2 | FPAVLQSSGLYSLSSVVTVPSSSLGTQTYICNVNHKPSNTKVDKK |
| | | VEPKSCDKTHTCPPCPAPEAAGGPCVFLFPPKPKDTLMISRTPEV |
| | | TCVVVDVSHEDPEVKFNWYVDGVEVHNAKTKPREEQYNSTYR |
| | | VVSVLTVLHQDWLNGKEYKCKVSNKALPAPIEKTISKAKGQPRE |
| | | PQVYTLPPSRDELTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 897 | Anti-PD-L1 | QVQLVQSGAEVKKPGSSVKVSCKTSGDTFSTAAISWVRQAPGQ |
| | mIgG2a | GLEWMGGIIPIFGKAHYAQKFQGRVTITADESTSTAYMELSSLRS |
| | LALA HC | EDTAVYFCARKFHFVSGSPFGMDVWGQGTTVTVSSAKTTAPSV |
| | | YPLAPVCGDTTGSSVTLGCLVKGYFPEPVTLTWNSGSLSSGVHT |
| | | FPAVLQSDLYTLSSSVTVTSSTWPSQSITCNVAHPASSTKVDKKIE |
| | | PRGPTIKPCPPCKCPAPNAAGGPSVFIFPPKIKDVLMISLSPIVTCV |
| | | VVDVSEDDPDVQISWFVNNVEVHTAQTQTHREDYNSTLRVVSA |
| | | LPIQHQDWMSGKEFKCKVNNKDLPAPIERTISKPKGSVRAPQVY |
| | | VLPPPEEEMTKKQVTLTCMVTDFMPEDIYVEWTNNGKTELNYK |
| | | NTEPVLDSDGSYFMYSKLRVEKKNWVERNSYSCSVVHEGLHNH |
| | | HTTKSFSRTPGK |
| 898 | Anti-PD-L1 | QVQLVQSGAEVKKPGSSVKVSCKTSGDTFSTAAISWVRQAPGQ |
| | mIgG2a | GLEWMGGIIPIFGKAHYAQKFQGRVTITADESTSTAYMELSSLRS |
| | LALA HC | EDTAVYFCARKFHFVSGSPFGMDVWGQGTTVTVSSAKTTAPSV |
| | v2 | YPLAPVCGDTTGSSVTLGCLVKGYFPEPVTLTWNSGSLSSGVHT |
| | | FPAVLQSDLYTLSSSVTVTSSTWPSQSITCNVAHPASSTKVDKKIE |
| | | PRGPTIKPCPPCKCPAPNAAGGPSVFIFPPKIKDVLMISLSPIVTCV |
| | | VVDVSEDDPDVQISWFVNNVEVHTAQTQTHREDYNSTLRVVSA |
| | | LPIQHQDWMSGKEFKCKVNNKDLPAPIERTISKPKGSVRAPQVY |
| | | VLPPPEEEMTKKQVTLTCMVTDFMPEDIYVEWTNNGKTELNYK |
| | | NTEPVLDSDGSYFMYSKLRVEKKNWVERNSYSCSVVHEGLHNH |
| | | HTTKSFSRTPG |
| 899 | Anti-PD-L1 | EIVLTQSPATLSLSPGERATLSCRASQSVSSYLAWYQQKPGQAPR |
| | mK LC | LLIYDASNRATGIPARFSGSGSGTDFTLTISSLEPEDFAVYYCQQR |
| | | SNWPTFGQGTKVEIKRADAAPTVSIFPPSSEQLTSGGASVVCFLN |
| | | NFYPKDINVKWKIDGSERQNGVLNSWTDQDSKDSTYSMSSTLTL |
| | | TKDEYERHNSYTCEATHKTSTSPIVKSFNRNEC |
| 900 | Anti-PD-L1 | QVQLVQSGAEVKKPGSSVKVSCKTSGDTFSTAAISWVRQAPGQ |
| | mIgG2a | GLEWMGGIIPIFGKAHYAQKFQGRVTITADESTSTAYMELSSLRS |
| | (engineered | EDTAVYFCARKFHFVSGSPFGMDVWGQGTTVTVSSAKTTAPSV |
| | cysteines) | YPLAPVCGDTTGSSVTLGCLVKGYFPEPVTLTWNSGSLSSGVHT |
| | LALA HC | FPAVLQSDLYTLSSSVTVTSSTWPSQSITCNVAHPASSTKVDKKIE |
| | | PRGPTIKPCPPCKCPAPNAAGGPCVFIFPPKIKDVLMISLSPIVTCV |
| | | VVDVSEDDPDVQISWFVNNVEVHTAQTQTHREDYNSTLRVVSA |
| | | LPIQHQDWMSGKEFKCKVNNKDLPAPIERTISKPKGSVRAPQVY |
| | | VLPPPEEEMTKKQVTLTCMVTDFMPEDIYVEWTNNGKTELNYK |
| | | NTEPVLDSDGSYFMYSKLRVEKKNWVERNSYSCSVVHEGLHNH |
| | | HTTKSFSRTPGK |
| 901 | Anti-PD-L1 | QVQLVQSGAEVKKPGSSVKVSCKTSGDTFSTAAISWVRQAPGQ |
| | mIgG2a | GLEWMGGIIPIFGKAHYAQKFQGRVTITADESTSTAYMELSSLRS |
| | (engineered | EDTAVYFCARKFHFVSGSPFGMDVWGQGTTVTVSSAKTTAPSV |
| | cysteines) | YPLAPVCGDTTGSSVTLGCLVKGYFPEPVTLTWNSGSLSSGVHT |
| | LALA HC | FPAVLQSDLYTLSSSVTVTSSTWPSQSITCNVAHPASSTKVDKKIE |
| | v2 | PRGPTIKPCPPCKCPAPNAAGGPCVFIFPPKIKDVLMISLSPIVTCV |
| | | VVDVSEDDPDVQISWFVNNVEVHTAQTQTHREDYNSTLRVVSA |
| | | LPIQHQDWMSGKEFKCKVNNKDLPAPIERTISKPKGSVRAPQVY |
| | | VLPPPEEEMTKKQVTLTCMVTDFMPEDIYVEWTNNGKTELNYK |
| | | NTEPVLDSDGSYFMYSKLRVEKKNWVERNSYSCSVVHEGLHNH |
| | | HTTKSFSRTPG |
| 902 | Anti-PD-L1 | TAAIS |
| | CDR-H1 | |
| 903 | Anti-PD-L1 | GIIPIFGKAHYAQKFQG |
| | CDR-H2 | |
| 904 | Anti-PD-L1 | KFHFVSGSPFGMDV |
| | CDR-H3 | |
| 905 | Anti-PD-L1 | RASQSVSSYLA |
| | CDR-L1 | |
| 906 | Anti-PD-L1 | DASNRAT |
| | CDR-L2 | |
| 907 | Anti-PD-L1 | QQRSNWPT |
| | CDR-L3 | |
| 908 | Anti-PD-L1 | QVQLVQSGAEVKKPGSSVKVSCKTSGDTFSTAAISWVRQAPGQ |
| | VH | GLEWMGGIIPIFGKAHYAQKFQGRVTITADESTSTAYMELSSLRS |
| | | EDTAVYFCARKFHFVSGSPFGMDVWGQGTTVTVSS |
| 909 | Anti-PD-L1 | EIVLTQSPATLSLSPGERATLSCRASQSVSSYLAWYQQKPGQAPR |
| | VL | LLIYDASNRATGIPARFSGSGSGTDFTLTISSLEPEDFAVYYCQQR |
| | | SNWPTFGQGTKVEIK |
| 910 | Anti-EphA2 | HYMMA |
| | CDR-H1 | |
| 911 | Anti-EphA2 | RIGPSGGPTHYADSVKG |
| | CDR-H2 | |
| 912 | Anti-EphA2 | YDSGYDYVAVAGPAEYFQH |
| | CDR-H3 | |
| 913 | Anti-EphA2 | RASQSISTWLA |
| | CDR-L1 | |
| 914 | Anti-EphA2 | KASNLHT |
| | CDR-L2 | |
| 915 | Anti-EphA2 | QQYNSYSRT |
| | CDR-L3 | |
| 916 | Anti-EphA2 | EVQLLESGGGLVQPGGSLRLSCAASGFTFSHYMMAWVRQAPGK |
| | VH | GLEWVSRIGPSGGPTHYADSVKGRFTISRDNSKNTLYLQMNSLR |
| | | AEDTAVYYCAGYDSGYDYVAVAGPAEYFQHWGQGTLVTVSS |
| 917 | Anti-EphA2 | DIQMTQSPSSLSASVGDRVTITCRASQSISTWLAWYQQKPGKAP |
| | VL | KLLIYKASNLHTGVPSRFSGSGSGTEFSLTISGLQPDDFATYYCQ |
| | | QYNSYSRTFGQGTKVEIK |
| 918 | Anti-EphA2 | EVQLLESGGGLVQPGGSLRLSCAASGFTFSHYMMAWVRQAPGK |
| | HC | GLEWVSRIGPSGGPTHYADSVKGRFTISRDNSKNTLYLQMNSLR |
| | | AEDTAVYYCAGYDSGYDYVAVAGPAEYFQHWGQGTLVTVSSA |
| | | STKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMI |
| | | SRTPEVTCVVVDVSHEDPEVKFNWYVDGVEVHNAKTKPREEQY |
| | | NSTYRVVSVLTVLHQDWLNGKEYKCKVSNKALPAPIEKTISKAK |
| | | GQPREPQVYTLPPSRDELTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 919 | Anti-EphA2 | EVQLLESGGGLVQPGGSLRLSCAASGFTFSHYMMAWVRQAPGK |
| | HC v2 | GLEWVSRIGPSGGPTHYADSVKGRFTISRDNSKNTLYLQMNSLR |
| | | AEDTAVYYCAGYDSGYDYVAVAGPAEYFQHWGQGTLVTVSSA |
| | | STKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMI |
| | | SRTPEVTCVVVDVSHEDPEVKFNWYVDGVEVHNAKTKPREEQY |
| | | NSTYRVVSVLTVLHQDWLNGKEYKCKVSNKALPAPIEKTISKAK |
| | | GQPREPQVYTLPPSRDELTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPG |
| 920 | Anti-EphA2 | DIQMTQSPSSLSASVGDRVTITCRASQSISTWLAWYQQKPGKAP |
| | LC | KLLIYKASNLHTGVPSRFSGSGSGTEFSLTISGLQPDDFATYYCQ |
| | | QYNSYSRTFGQGTKVEIKRTVAAPSVFIFPPSDEQLKSGTASVVC |
| | | LLNNFYPREAKVQWKVDNALQSGNSQESVTEQDS |
| | | KDSTYSLSSTLTLSKADYEKHKVYACEVTHQGLSSPVTKSFNRG |
| | | EC |
| 921 | Anti-EphA2 | EVQLLESGGGLVQPGGSLRLSCAASGFTFSHYMMAWVRQAPGK |
| | mIgG2a HC | GLEWVSRIGPSGGPTHYADSVKGRFTISRDNSKNTLYLQMNSLR |
| | | AEDTAVYYCAGYDSGYDYVAVAGPAEYFQHWGQGTLVTVSSA |
| | | KTTAPSVYPLAPVCGDTTGSSVTLGCLVKGYFPEPVTLTWNSGS |
| | | LSSGVHTFPAVLQSDLYTLSSSVTVTSSTWPSQSITCNVAHPASST |
| | | KVDKKIEPRGPTIKPCPPCKCPAPNLLGGPSVFIFPPKIKDVLMISL |
| | | SPIVTCVVVDVSEDDPDVQISWFVNNVEVHTAQTQTHREDYNST |
| | | LRVVSALPIQHQDWMSGKEFKCKVNNKDLPAPIERTISKPKGSV |
| | | RAPQVYVLPPPEEEMTKKQVTLTCMVTDFMPEDIYVEWTNNGK |
| | | TELNYKNTEPVLDSDGSYFMYSKLRVEKKNWVERNSYSCSVVH |
| | | EGLHNHHTTKSFSRTPGK |
| 922 | Anti-EphA2 | EVQLLESGGGLVQPGGSLRLSCAASGFTFSHYMMAWVRQAPGK |
| | mIgG2a HC | GLEWVSRIGPSGGPTHYADSVKGRFTISRDNSKNTLYLQMNSLR |
| | v2 | AEDTAVYYCAGYDSGYDYVAVAGPAEYFQHWGQGTLVTVSSA |
| | | KTTAPSVYPLAPVCGDTTGSSVTLGCLVKGYFPEPVTLTWNSGS |
| | | LSSGVHTFPAVLQSDLYTLSSSVTVTSSTWPSQSITCNVAHPASST |
| | | KVDKKIEPRGPTIKPCPPCKCPAPNLLGGPSVFIFPPKIKDVLMISL |
| | | SPIVTCVVVDVSEDDPDVQISWFVNNVEVHTAQTQTHREDYNST |
| | | LRVVSALPIQHQDWMSGKEFKCKVNNKDLPAPIERTISKPKGSV |
| | | RAPQVYVLPPPEEEMTKKQVTLTCMVTDFMPEDIYVEWTNNGK |
| | | TELNYKNTEPVLDSDGSYFMYSKLRVEKKNWVERNSYSCSVVH |
| | | EGLHNHHTTKSFSRTPG |
| 923 | Anti-EphA2 | DIQMTQSPSSLSASVGDRVTITCRASQSISTWLAWYQQKPGKAP |
| | mIgG2a LC | KLLIYKASNLHTGVPSRFSGSGSGTEFSLTISGLQPDDFATYYCQ |
| | | QYNSYSRTFGQGTKVEIKRADAAPTVSIFPPSSEQLTSGGASVVC |
| | | FLNNFYPKDINVKWKIDGSERQNGVLNSWTDQDSKDSTYSMSS |
| | | TLTLTKDEYERHNSYTCEATHKTSTSPIVKSFNRNEC |
| 924 | Anti-EphA2 | EVQLLESGGGLVQPGGSLRLSCAASGFTFSHYMMAWVRQAPGK |
| | mIgG2a | GLEWVSRIGPSGGPTHYADSVKGRFTISRDNSKNTLYLQMNSLR |
| | LALAPG | AEDTAVYYCAGYDSGYDYVAVAGPAEYFQHWGQGTLVTVSSA |
| | HC | KTTAPSVYPLAPVCGDTTGSSVTLGCLVKGYFPEPVTLTWNSGS |
| | | LSSGVHTFPAVLQSDLYTLSSSVTVTSSTWPSQSITCNVAHPASST |
| | | KVDKKIEPRGPTIKPCPPCKCPAPNAAGGPSVFIFPPKIKDVLMISL |
| | | SPIVTCVVVDVSEDDPDVQISWFVNNVEVHTAQTQTHREDYNST |
| | | LRVVSALPIQHQDWMSGKEFKCKVNNKDLGAPIERTISKPKGSV |
| | | RAPQVYVLPPPEEEMTKKQVTLTCMVTDFMPEDIYVEWTNNGK |
| | | TELNYKNTEPVLDSDGSYFMYSKLRVEKKNWVERNSYSCSVVH |
| | | EGLHNHHTTKSFSRTPGK |
| 925 | Anti-EphA2 | EVQLLESGGGLVQPGGSLRLSCAASGFTFSHYMMAWVRQAPGK |
| | mIgG2a | GLEWVSRIGPSGGPTHYADSVKGRFTISRDNSKNTLYLQMNSLR |
| | LALAPG | AEDTAVYYCAGYDSGYDYVAVAGPAEYFQHWGQGTLVTVSSA |
| | HC v2 | KTTAPSVYPLAPVCGDTTGSSVTLGCLVKGYFPEPVTLTWNSGS |
| | | LSSGVHTFPAVLQSDLYTLSSSVTVTSSTWPSQSITCNVAHPASST |
| | | KVDKKIEPRGPTIKPCPPCKCPAPNAAGGPSVFIFPPKIKDVLMISL |
| | | SPIVTCVVVDVSEDDPDVQISWFVNNVEVHTAQTQTHREDYNST |
| | | LRVVSALPIQHQDWMSGKEFKCKVNNKDLGAPIERTISKPKGSV |
| | | RAPQVYVLPPPEEEMTKKQVTLTCMVTDFMPEDIYVEWTNNGK |
| | | TELNYKNTEPVLDSDGSYFMYSKLRVEKKNWVERNSYSCSVVH |
| | | EGLHNHHTTKSFSRTPG |
| 926 | Anti-EphA2 | DIQMTQSPSSLSASVGDRVTITCRASQSISTWLAWYQQKPGKAP |
| | mIgG2a | KLLIYKASNLHTGVPSRFSGSGSGTEFSLTISGLQPDDFATYYCQ |
| | LALAPG | QYNSYSRTFGQGTKVEIKRADAAPTVSIFPPSSEQLTSGGASVVC |
| | LC | FLNNFYPKDINVKWKIDGSERQNGVLNSWTDQDSKDSTYSMSS |
| | | TLTLTKDEYERHNSYTCEATHKTSTSPIVKSFNRNEC |
| 927 | Anti-CD228 | SGYWN |
| | CDR-H1 | |
| 928 | Anti-CD228 | YISDSGITYYNPSLKS |
| | CDR-H2 | |
| 929 | Anti-CD228 | RTLATYYAMDY |
| | CDR-H3 | |
| 930 | Anti-CD228 | RASQSLVHSDGNTYLH |
| | CDR-L1 | |
| 931 | Anti-CD228 | RVSNRFS |
| | CDR-L2 | |
| 932 | Anti-CD228 | SQSTHVPPT |
| | CDR-L3 | |
| 933 | Anti-CD228 | QVQLQESGPGLVKPSETLSLTCTVSGDSITSGYWNWIRQPPGKGL |
| | VH | EYIGYISDSGITYYNPSLKSRVTISRDTSKNQYSLKLSSVTAADTA |
| | | VYYCARRTLATYYAMDYWGQGTLVTVSS |
| 934 | Anti-CD228 | DFVMTQSPLSLPVTLGQPASISCRASQSLVHSDGNTYLHWYQQR |
| | VL | PGQSPRLLIYRVSNRFSGVPDRFSGSGSGTDFTLKISRVEAEDVG |
| | | VYYCSQSTHVPPTFGQGTKLEIK |
| 935 | Anti-CD228 | QVQLQESGPGLVKPSETLSLTCTVSGDSITSGYWNWIRQPPGKGL |
| | HC | EYIGYISDSGITYYNPSLKSRVTISRDTSKNQYSLKLSSVTAADTA |
| | | VYYCARRTLATYYAMDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPGK |
| 936 | Anti-CD228 | QVQLQESGPGLVKPSETLSLTCTVSGDSITSGYWNWIRQPPGKGL |
| | HC v2 | EYIGYISDSGITYYNPSLKSRVTISRDTSKNQYSLKLSSVTAADTA |
| | | VYYCARRTLATYYAMDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPG |
| 937 | Anti-CD228 | DFVMTQSPLSLPVTLGQPASISCRASQSLVHSDGNTYLHWYQQR |
| | LC | PGQSPRLLIYRVSNRFSGVPDRFSGSGSGTDFTLKISRVEAEDVG |
| | | VYYCSQSTHVPPTFGQGTKLEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| 938 | Anti-CD228 | QVQLQESGPGLVKPSETLSLTCTVSGDSITSGYWNWIRQPPGKGL |
| | LALAKA | EYIGYISDSGITYYNPSLKSRVTISRDTSKNQYSLKLSSVTAADTA |
| | HC | VYYCARRTLATYYAMDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKKVEPKSCD |
| | | KTHTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCAVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPGK |
| 939 | Anti-CD228 | QVQLQESGPGLVKPSETLSLTCTVSGDSITSGYWNWIRQPPGKGL |
| | LALAKA | EYIGYISDSGITYYNPSLKSRVTISRDTSKNQYSLKLSSVTAADTA |
| | HC v2 | VYYCARRTLATYYAMDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKKVEPKSCD |
| | | KTHTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCAVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPG |
| 940 | Anti-CD228 | DFVMTQSPLSLPVTLGQPASISCRASQSLVHSDGNTYLHWYQQR |
| | LALAKA | PGQSPRLLIYRVSNRFSGVPDRFSGSGSGTDFTLKISRVEAEDVG |
| | LC | VYYCSQSTHVPPTFGQGTKLEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| 941 | Anti-avB6 | DYNVN |
| | HC CDR-H1 | |
| 942 | Anti-AvB6 | VINPKYGTTRYNQKFKG |
| | HC CDR-H2 | |
| 943 | Anti-AvB6 | GLNAWDY |
| | HC CDR-H3 | |
| 944 | Anti-AvB6 | GASENIYGALN |
| | LG CDR-L1 | |
| 945 | Anti-AvB6 | GATNLED |
| | LG CDR-L2 | |
| 946 | Anti-AvB6 | QNVLTTPYT |
| | LG CDR-L3 | |
| 947 | Anti-AvB6 | QFQLVQSGAEVKKPGASVKVSCKASGYSFTDYNVNWVRQAPG |
| | HC VH | QGLEWIGVINPKYGTTRYNQKFKGRATLTVDKSTSTAYMELSSL |
| | | RSEDTAVYYCTRGLNAWDYWGQGTLVTVSS |
| 948 | Anti-AvB6 | DIQMTQSPSSLSASVGDRVTITCGASENIYGALNWYQQKPGKAP |
| | LG VL | KLLIYGATNLEDGVPSRFSGSGSGRDYTFTISSLQPEDIATYYCQN |
| | | VLTTPYTFGQGTKLEIK |
| 949 | Anti-AvB6 | QFQLVQSGAEVKKPGASVKVSCKASGYSFTDYNVNWVRQAPG |
| | HC | QGLEWIGVINPKYGTTRYNQKFKGRATLTVDKSTSTAYMELSSL |
| | | RSEDTAVYYCTRGLNAWDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 950 | Anti-AvB6 | QFQLVQSGAEVKKPGASVKVSCKASGYSFTDYNVNWVRQAPG |
| | HC v2 | QGLEWIGVINPKYGTTRYNQKFKGRATLTVDKSTSTAYMELSSL |
| | | RSEDTAVYYCTRGLNAWDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG |
| 951 | Anti-AvB6 | DIQMTQSPSSLSASVGDRVTITCGASENIYGALNWYQQKPGKAP |
| | LG LC | KLLIYGATNLEDGVPSRFSGSGSGRDYTFTISSLQPEDIATYYCQN |
| | | VLTTPYTFGQGTKLEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 952 | Anti-AvB6 | QFQLVQSGAEVKKPGASVKVSCKASGYSFTDYNVNWVRQAPG |
| | LALAKA | QGLEWIGVINPKYGTTRYNQKFKGRATLTVDKSTSTAYMELSSL |
| | HC | RSEDTAVYYCTRGLNAWDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCAVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 953 | Anti-AvB6 | QFQLVQSGAEVKKPGASVKVSCKASGYSFTDYNVNWVRQAPG |
| | LALAKA | QGLEWIGVINPKYGTTRYNQKFKGRATLTVDKSTSTAYMELSSL |
| | HC v2 | RSEDTAVYYCTRGLNAWDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCAVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG |
| 954 | Anti-AvB6 | DIQMTQSPSSLSASVGDRVTITCGASENIYGALNWYQQKPGKAP |
| | LG | KLLIYGATNLEDGVPSRFSGSGSGRDYTFTISSLQPEDIATYYCQN |
| | LALAKA | VLTTPYTFGQGTKLEIKRTVAAPSVFIFPPSDEQLKSGTASVVCLL |
| | LC | NNFYPREAKVQWKVDNALQSGNSQESVTEQDSKDSTYSLSSTLT |
| | | LSKADYEKHKVYACEVTHQGLSSPVTKSFNRGEC |
| 955 | Anti-B7H4 | SGSYYWG |
| | CDR-H1 | |
| 956 | Anti-B7H4 | NIYYSGSTYYNPSLRS |
| | CDR-H2 | |
| 957 | Anti-B7H4 | EGSYPNQFDP |
| | CDR-H3 | |
| 958 | Anti-B7H4 | RASQSVSSNLA |
| | CDR-L1 | |
| 959 | Anti-B7H4 | GASTRAT |
| | CDR-L2 | |
| 960 | Anti-B7H4 | QQYHSFPFT |
| | CDR-L3 | |
| 961 | Anti-B7H4 | QLQLQESGPGLVKPSETLSLTCTVSGGSIKSGSYYWGWIRQPPGK |
| | VH | GLEWIGNIYYSGSTYYNPSLRSRVTISVDTSKNQFSLKLSSVTAA |
| | | DTAVYYCAREGSYPNQFDPWGQGTLVTVSS |
| 962 | Anti-B7H4 | EIVMTQSPATLSVSPGERATLSCRASQSVSSNLAWYQQKPGQAP |
| | VL | RLLIYGASTRATGIPARFSGSGSGTEFTLTISSLQSEDFAVYYCQQ |
| | | YHSFPFTFGGGTKVEIK |
| 963 | Anti-B7H4 | QLQLQESGPGLVKPSETLSLTCTVSGGSIKSGSYYWGWIRQPPGK |
| | HC | GLEWIGNIYYSGSTYYNPSLRSRVTISVDTSKNQFSLKLSSVTAA |
| | | DTAVYYCAREGSYPNQFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 964 | Anti-B7H4 | QLQLQESGPGLVKPSETLSLTCTVSGGSIKSGSYYWGWIRQPPGK |
| | HC v2 | GLEWIGNIYYSGSTYYNPSLRSRVTISVDTSKNQFSLKLSSVTAA |
| | | DTAVYYCAREGSYPNQFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG |
| 965 | Anti-B7H4 | EIVMTQSPATLSVSPGERATLSCRASQSVSSNLAWYQQKPGQAP |
| | LC | RLLIYGASTRATGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 966 | Anti-B7H4 | QLQLQESGPGLVKPSETLSLTCTVSGGSIKSGSYYWGWIRQPPGK |
| | LALAKA | GLEWIGNIYYSGSTYYNPSLRSRVTISVDTSKNQFSLKLSSVTAA |
| | HC | DTAVYYCAREGSYPNQFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCAVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 967 | Anti-B7H4 | QLQLQESGPGLVKPSETLSLTCTVSGGSIKSGSYYWGWIRQPPGK |
| | LALAKA | GLEWIGNIYYSGSTYYNPSLRSRVTISVDTSKNQFSLKLSSVTAA |
| | HC v2 | DTAVYYCAREGSYPNQFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCAVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPG |
| 968 | Anti-B7H4 | EIVMTQSPATLSVSPGERATLSCRASQSVSSNLAWYQQKPGQAP |
| | LALAKA | RLLIYGASTRATGIPARFSGSGSGTEFTLTISSLQSEDFAVYYCQQ |
| | LC | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 969 | Anti-B7H4 | GSISSSSYYWG |
| | CDR-H1 | |
| 970 | Anti-B7H4 | NIYYSGSTYYNPSLKS |
| | CDR-H2 | |
| 971 | Anti-B7H4 | AREGSYPNWFDP |
| | CDR-H3 | |
| 972 | Anti-B7H4 | RASQSVSSNLA |
| | CDR-L1 | |
| 973 | Anti-B7H4 | GASTRAT |
| | CDR-L2 | |
| 974 | Anti-B7H4 | QQYHSFPFT |
| | CDR-L3 | |
| 975 | Anti-B7H4 | QLQLQESGPGLVKPSETLSLTCTVSGGSISSSSYYWGWIRQPPGK |
| | VH | GLEWIGNIYYSGSTYYNPSLKSRVTISVDTSKNQFSLKLSSVTAA |
| | | DTAVYYCAREGSYPNWFDPWGQGTLVTVSS |
| 976 | Anti-B7H4 | EIVMTQSPATLSVSPGERATLSCRASQSVSSNLAWYQQKPGQAP |
| | VL | RLLIYGASTRATGIPARFSGSGSGTEFTLTISSLQSEDFAVYYCQQ |
| | | YHSFPFTFGGGTKVEIK |
| 977 | Anti-B7H4 | GSIKSGSHYWG |
| | CDR-H1 | |
| 978 | Anti-B7H4 | NIYYSGSTYYNPSLRS |
| | CDR-H2 | |
| 979 | Anti-B7H4 | AREGSYPNWFDP |
| | CDR-H3 | |
| 980 | Anti-B7H4 | RASQSVSSNLA |
| | CDR-L1 | |
| 981 | Anti-B7H4 | GASTRAT |
| | CDR-L2 | |
| 982 | Anti-B7H4 | QQYHSFPFT |
| | CDR-L3 | |
| 983 | Anti-B7H4 | QLQLQESGPGLVKPSETLSLTCTVSGGSIKSGSHYWGWIRQPPGK |
| | VH | GLEWIGNIYYSGSTYYNPSLRSRVTISVDTSKNQFSLKLSSVTAA |
| | | DTAVYYCAREGSYPNWFDPWGQGTLVTVSS |
| 984 | Anti-B7H4 | EIVMTQSPATLSVSPGERATLSCRASQSVSSNLAWYQQKPGQAP |
| | VL | RLLIYGASTRATGIPARFSGSGSGTEFTLTISSLQSEDFAVYYCQQ |
| | | YHSFPFTFGGGTKVEIK |
| 985 | Anti-B7H4 | GSIKSGSHYWG |
| | CDR-H1 | |
| 986 | Anti-B7H4 | NIYYSGSTYYNPSLKS |
| | CDR-H2 | |
| 987 | Anti-B7H4 | AREGSYPNWLDP |
| | CDR-H3 | |
| 988 | Anti-B7H4 | RASQSVSSNLA |
| | CDR-L1 | |
| 989 | Anti-B7H4 | GASTRAT |
| | CDR-L2 | |
| 990 | Anti-B7H4 | QQYHSFPFT |
| | CDR-L3 | |
| 991 | Anti-B7H4 | QLQLQESGPGLVKPSETLSLTCTVSGGSIKSGSHYWGWIRQPPGK |
| | VH | GLEWIGNIYYSGSTYYNPSLKSRVTISVDTSKNQFSLKLSSVTAA |
| | | DTAVYYCAREGSYPNWLDPWGQGTLVTVSS |
| 992 | Anti-B7H4 | EIVMTQSPATLSVSPGERATLSCRASQSVSSNLAWYQQKPGQAP |
| | VL | RLLIYGASTRATGIPARFSGSGSGTEFTLTISSLQSEDFAVYYCQQ |
| | | YHSFPFTFGGGTKVEIK |
| 993 | Anti-B7H4 | GSIKSGSYYWG |
| | CDR-H1 | |
| 994 | Anti-B7H4 | NIYYSGSTYYNPSLKS |
| | CDR-H2 | |
| 995 | Anti-B7H4 | AREGSYPNQFDP |
| | CDR-H3 | |
| 996 | Anti-B7H4 | RASQSVSSNLA |
| | CDR-L1 | |
| 997 | Anti-B7H4 | GASTRAT |
| | CDR-L2 | |
| 998 | Anti-B7H4 | QQYHSFPFT |
| | CDR-L3 | |
| 999 | Anti-B7H4 | QLQLQESGPGLVKPSETLSLTCTVSGGSIKSGSYYWGWIRQPPGK |
| | VH | GLEWIGNIYYSGSTYYNPSLKSRVTISVDTSKNQFSLKLSSVTAA |
| | | DTAVYYCAREGSYPNQFDPWGQGILVTVSS |
| 1000 | Anti-B7H4 | EIVMTQSPATLSVSPGERATLSCRASQSVSSNLAWYQQKPGQAP |
| | VL | RLLIYGASTRATGIPARFSGSGSGTEFTLTISSLQSEDFAVYYCQQ |
| | | YHSFPFTFGGGTKVEIK |
| 1001 | Anti-B7H4 | GSIKSGSHYWG |
| | CDR-H1 | |
| 1002 | Anti-B7H4 | NIYYSGSTYYNPSLKS |
| | CDR-H2 | |
| 1003 | Anti-B7H4 | AREGSYPNWFDP |
| | CDR-H3 | |
| 1004 | Anti-B7H4 | RASQSVSTNLA |
| | CDR-L1 | |
| 1005 | Anti-B7H4 | DASARVT |
| | CDR-L2 | |
| 1006 | Anti-B7H4 | QQYHSFPFT |
| | CDR-L3 | |
| 1007 | Anti-B7H4 | QLQLQESGPGLVKPSETLSLTCTVSGGSIKSGSHYWGWIRQPPGK |
| | VH | GLEWIGNIYYSGSTYYNPSLKSRVTMSVDTSKNQFSLKLSSVTA |
| | | ADTAVYYCAREGSYPNWFDPWGQGTLVTVSS |
| 1008 | Anti-B7H4 | EIVMTQSPATLSVSPGERATLSCRASQSVSTNLAWYQQKPGQAP |
| | VL | RLLIYDASARVTGIPARFSGSGSGTEFTLTISSLQSEDFAVYYCQQ |
| | | YHSFPFTFGGGTKVEIK |
| 1009 | Anti-B7H4 | GSISSSSYYWG |
| | CDR-H1 | |
| 1010 | Anti-B7H4 | NIYYSGSTYYNPSLKS |
| | CDR-H2 | |
| 1011 | Anti-B7H4 | AREGSYTTVLNV |
| | CDR-H3 | |
| 1012 | Anti-B7H4 | RASQSVSSSYLA |
| | CDR-L1 | |
| 1013 | Anti-B7H4 | GASSRAT |
| | CDR-L2 | |
| 1014 | Anti-B7H4 | QQAASYPLT |
| | CDR-L3 | |
| 1015 | Anti-B7H4 | QLQLQESGPGLVKPSETLSLTCTVSGGSISSSSYYWGWIRQPPGK |
| | VH | GLEWIGNIYYSGSTYYNPSLKSRVTISVDTSKNQFSLKLSSVTAA |
| | | DTAVYYCAREGSYTTVLNVWGQGTMVTVSS |
| 1016 | Anti-B7H4 | EIVLTQSPGTLSLSPGERATLSCRASQSVSSSYLAWYQQKPGQAP |
| | VL | RLLIYGASSRATGIPDRFSGSGSGTDFTLTISRLEPEDFAVYYCQQ |
| | | AASYPLTFGGGTKVEIK |
| 1017 | Anti-B7H4 | GSIGRGSYYWG |
| | CDR-H1 | |
| 1018 | Anti-B7H4 | NIYYSGSTYYNPSLKS |
| | CDR-H2 | |
| 1019 | Anti-B7H4 | AREGSYTTVLNV |
| | CDR-H3 | |
| 1020 | Anti-B7H4 | RASQSVASSHLA |
| | CDR-L1 | |
| 1021 | Anti-B7H4 | DAVSRAT |
| | CDR-L2 | |
| 1022 | Anti-B7H4 | QQAASYPLT |
| | CDR-L3 | |
| 1023 | Anti-B7H4 | QLQLQESGPGLVKPSETLSLTCTVSGGSIGRGSYYWGWIRQPPG |
| | VH | KGLEWIGNIYYSGSTYYNPSLKSRVTISVDTSKNQFSLKLSSVTA |
| | | ADTAVYYCAREGSYTTVLNVWGQGTMVTVSS |
| 1024 | Anti-B7H4 | EIVLTQSPGTLSLSPGERATLSCRASQSVASSHLAWYQQKPGQAP |
| | VL | RLLIYDAVSRATGIPDRFSGSGSGTDFTLTISRLEPEDFAVYYCQQ |
| | | AASYPLTFGGGTKVEIK |
| 1025 | Anti-B7H4 | GSISSGGYYWS |
| | CDR-H1 | |
| 1026 | Anti-B7H4 | NIYYSGSTYYNPSLKS |
| | CDR-H2 | |
| 1027 | Anti-B7H4 | ARESSTISADFDL |
| | CDR-H3 | |
| 1028 | Anti-B7H4 | RASQGISRWLA |
| | CDR-L1 | |
| 1029 | Anti-B7H4 | AASSLQS |
| | CDR-L2 | |
| 1030 | Anti-B7H4 | QQAHTFPYT |
| | CDR-L3 | |
| 1031 | Anti-B7H4 | QVQLQESGPGLVKPSQTLSLTCTVSGGSISSGGYYWSWIRQHPG |
| | VH | KGLEWIGNIYYSGSTYYNPSLKSRVTISVDTSKNQFSLKLSSVTA |
| | | ADTAVYYCARESSTISADFDLWGRGTLVTVSS |
| 1032 | Anti-B7H4 | DIQMTQSPSSVSASVGDRVTITCRASQGISRWLAWYQQKPGKAP |
| | VL | KLLIYAASSLQSGVPSRFSGSGSGTDFTLTISSLQPEDFATYYCQQ |
| | | AHTFPYTFGGGTKVEIK |
| 1033 | Anti-B7H4 | GSISHGGYYWS |
| | CDR-H1 | |
| 1034 | Anti-B7H4 | NIYYSGSTYYNPSLKS |
| | CDR-H2 | |
| 1035 | Anti-B7H4 | ARESSTISADFDL |
| | CDR-H3 | |
| 1036 | Anti-B7H4 | RASQGISRWLA |
| | CDR-L1 | |
| 1037 | Anti-B7H4 | AASSLQS |
| | CDR-L2 | |
| 1038 | Anti-B7H4 | QQAHTFPYT |
| | CDR-L3 | |
| 1039 | Anti-B7H4 | QLQLQESGPGLVKPSETLSLTCTASGGSISHGGYYWSWIRQHPG |
| | VH | KGLEWIGNIYYSGSTYYNPSLKSRVTMSVDTSKNQFSLKLSSVT |
| | | AADTAVYYCARESSTISADFDLWGRGTLVTVSS |
| 1040 | Anti-B7H4 | DIQMTQSPSSVSASVGDRVTITCRASQGISRWLAWYQQKPGKAP |
| | VL | KLLIYAASSLQSGVPSRFSGSGSGTDFTLTISSLQPEDFATYYCQQ |
| | | AHTFPYTFGGGTKVEIK |
| 1041 | Anti-B7H4 | GSISSGGYYWS |
| | CDR-H1 | |
| 1042 | Anti-B7H4 | NIYYSGSTYYNPSLKS |
| | CDR-H2 | |
| 1043 | Anti-B7H4 | ARGLSTIDEAFDP |
| | CDR-H3 | |
| 1044 | Anti-B7H4 | RASQSISSWLA |
| | CDR-L1 | |
| 1045 | Anti-B7H4 | KASSLES |
| | CDR-L2 | |
| 1046 | Anti-B7H4 | QQDNSYPYT |
| | CDR-L3 | |
| 1047 | Anti-B7H4 | QVQLQESGPGLVKPSQTLSLTCTVSGGSISSGGYYWSWIRQHPG |
| | VH | KGLEWIGNIYYSGSTYYNPSLKSRVTISVDTSKNQFSLKLSSVTA |
| | | ADTAVYYCARGLSTIDEAFDPWGQGTLVTVSS |
| 1048 | Anti-B7H4 | DIQMTQSPSTLSASVGDRVTITCRASQSISSWLAWYQQKPGKAP |
| | VL | KLLIYKASSLESGVPSRFSGSGSGTEFTLTISSLQPDDFATYYCQQ |
| | | DNSYPYTFGGGTKVEIK |
| 1049 | Anti-B7H4 | GSISDGSYYWS |
| | CDR-H1 | |
| 1050 | Anti-B7H4 | NIYYSGSTYYNPSLRS |
| | CDR-H2 | |
| 1051 | Anti-B7H4 | ARGLSTIDEAFDP |
| | CDR-H3 | |
| 1052 | Anti-B7H4 | RASQSISSWLA |
| | CDR-L1 | |
| 1053 | Anti-B7H4 | KASSLES |
| | CDR-L2 | |
| 1054 | Anti-B7H4 | QQDNSYPYT |
| | CDR-L3 | |
| 1055 | Anti-B7H4 | QLQLQESGPGLVKPSETLSLTCTVSGGSISDGSYYWSWIRQHPGK |
| | VH | GLEWIGNIYYSGSTYYNPSLRSRVTMSVDTSKNQFSLKLSSVTAA |
| | | DTAVYYCARGLSTIDEAFDPWGQGTLVTVSS |
| 1056 | Anti-B7H4 | DIQMTQSPSTLSASVGDRVTITCRASQSISSWLAWYQQKPGKAP |
| | VL | KLLIYKASSLESGVPSRFSGSGSGTEFTLTISSLQPDDFATYYCQQ |
| | | DNSYPYTFGGGTKVEIK |
| 1057 | Anti-B7H4 | GSISDGSYYWS |
| | CDR-H1 | |
| 1058 | Anti-B7H4 | NIYYSGSTYYNPSLRS |
| | CDR-H2 | |
| 1059 | Anti-B7H4 | ARGLSTIDEAFDP |
| | CDR-H3 | |
| 1060 | Anti-B7H4 | RASKSISSWLA |
| | CDR-L1 | |
| 1061 | Anti-B7H4 | EASSLHS |
| | CDR-L2 | |
| 1062 | Anti-B7H4 | QQDNSYPYT |
| | CDR-L3 | |
| 1063 | Anti-B7H4 | QVQLQESGPGLVKPSQTLSLTCTVSGGSISDGSYYWSWIRQHPG |
| | VH | KGLEWIGNIYYSGSTYYNPSLRSRVTMSVDTSKNQFSLKLSSVTA |
| | | ADTAVYYCARGLSTIDEAFDPWGQGTLVTVSS |
| 1064 | Anti-B7H4 | DIQMTQSPSTLSASVGDRVTITCRASKSISSWLAWYQQKPGKAP |
| | VL | KLLIYEASSLHSGVPSRFSGSGSGTEFTLTISSLQPDDFATYYCQQ |
| | | DNSYPYTFGGGTKVEIK |
| 1065 | Anti-B7H4 | GSISSYYWS |
| | CDR-H1 | |
| 1066 | Anti-B7H4 | YIYSSGSTNYNPSLKS |
| | CDR-H2 | |
| 1067 | Anti-B7H4 | ARGSGQYAAPDYGMD |
| | CDR-H3 | |
| 1068 | Anti-B7H4 | RASQSISSWLA |
| | CDR-L1 | |
| 1069 | Anti-B7H4 | KASSLES |
| | CDR-L2 | |
| 1070 | Anti-B7H4 | QQDNSFPFT |
| | CDR-L3 | |
| 1071 | Anti-B7H4 | QVQLQESGPGLVKPSETLSLTCTVSGGSISSYYWSWIRQPPGKGL |
| | VH | EWIGYIYSSGSTNYNPSLKSRVTISVDTSKNQFSLKLSSVTAADT |
| | | AVYYCARGSGQYAAPDYGMDVWGQGTTVTVSS |
| 1072 | Anti-B7H4 | DIQMTQSPSTLSASVGDRVTITCRASQSISSWLAWYQQKPGKAP |
| | VL | KLLIYKASSLESGVPSRFSGSGSGTEFTLTISSLQPDDFATYYCQQ |
| | | DNSFPFTFGGGTKVEIK |
| 1073 | Anti-B7H4 | GSIISYYWG |
| | CDR-H1 | |
| 1074 | Anti-B7H4 | YIYSSGSTSYNPSLKS |
| | CDR-H2 | |
| 1075 | Anti-B7H4 | ARGSGLYAAPDYGLDV |
| | CDR-H3 | |
| 1076 | Anti-B7H4 | RASQSISSWLA |
| | CDR-L1 | |
| 1077 | Anti-B7H4 | KASSLES |
| | CDR-L2 | |
| 1078 | Anti-B7H4 | QQDNSFPFT |
| | CDR-L3 | |
| 1079 | Anti-B7H4 | QVQLQESGPGLVKPSETLSLTCTVSGGSIISYYWGWIRQPPGKGL |
| | VH | EWIGYIYSSGSTSYNPSLKSRVTISVDTSKNQFSLKLSSVTAADTA |
| | | VYYCARGSGLYAAPDYGLDVWGQGTTVTVSS |
| 1080 | Anti-B7H4 | DIQMTQSPSTLSASVGDRVTITCRASQSISSWLAWYQQKPGKAP |
| | VL | KLLIYKASSLESGVPSRFSGSGSGTEFTLTISSLQPDDFATYYCQQ |
| | | DNSFPFTFGGGTKVEIK |
| 1081 | Anti-B7H4 | FTFSSYAMS |
| | CDR-H1 | |
| 1082 | Anti-B7H4 | TISGSGGSTYYADSVKG |
| | CDR-H2 | |
| 1083 | Anti-B7H4 | ARGAGHYDLVGRY |
| | CDR-H3 | |
| 1084 | Anti-B7H4 | RASQSISSYLN |
| | CDR-L1 | |
| 1085 | Anti-B7H4 | AASSLQS |
| | CDR-L2 | |
| 1086 | Anti-B7H4 | QQLYSLPPT |
| | CDR-L3 | |
| 1087 | Anti-B7H4 | EVQLLESGGGLVQPGGSLRLSCAASGFTFSSYAMSWVRQAPGK |
| | VH | GLEWVSTISGSGGSTYYADSVKGRFTISRDNSKNTLYLQMNSLR |
| | | AEDTAVYYCARGAGHYDLVGRYWGQGTLVTVSS |
| 1088 | Anti-B7H4 | DIQMTQSPSSLSASVGDRVTITCRASQSISSYLNWYQQKPGKAPK |
| | VL | LLIYAASSLQSGVPSRFSGSGSGTDFTLTISSLQPEDFATYYCQQL |
| | | YSLPPTFGGGTKVEIK |
| 1089 | Anti-B7H4 | FTFSSYAMS |
| | CDR-H1 | |
| 1090 | Anti-B7H4 | AISGSGGSTYYADSVKG |
| | CDR-H2 | |
| 1091 | Anti-B7H4 | ARVGFRALNY |
| | CDR-H3 | |
| 1092 | Anti-B7H4 | RASQDISSWLA |
| | CDR-L1 | |
| 1093 | Anti-B7H4 | AASSLQS |
| | CDR-L2 | |
| 1094 | Anti-B7H4 | QQATSYPPWT |
| | CDR-L3 | |
| 1095 | Anti-B7H4 | EVQLLESGGGLVQPGGSLRLSCAASGFTFSSYAMSWVRQAPGK |
| | VH | GLEWVSAISGSGGSTYYADSVKGRFTISRDNSKNTLYLQMNSLR |
| | | AEDTAVYYCARVGFRALNYWGQGTTVTVSS |
| 1096 | Anti-B7H4 | DIQLTQSPSSVSASVGDRVTITCRASQDISSWLAWYQQKPGKAP |
| | VL | KLLIYAASSLQSGVPSRFSGSGSGTDFTLTISSLQPEDFATYYCQQ |
| | | ATSYPPWTFGGGTKVEIK |
| 1097 | Anti-B7H4 | GTFSSYAIS |
| | CDR-H1 | |
| 1098 | Anti-B7H4 | GIIPIFGTASYAQKFQG |
| | CDR-H2 | |
| 1099 | Anti-B7H4 | ARQQYDGRRYFGL |
| | CDR-H3 | |
| 1100 | Anti-B7H4 | RASQSVSSNLA |
| | CDR-L1 | |
| 1101 | Anti-B7H4 | SASTRAT |
| | CDR-L2 | |
| 1102 | Anti-B7H4 | QQVNVWPPT |
| | CDR-L3 | |
| 1103 | Anti-B7H4 | QVQLVQSGAEVKKPGSSVKVSCKASGGTFSSYAISWVRQAPGQ |
| | VH | GLEWMGGIIPIFGTASYAQKFQGRVTITADESTSTAYMELSSLRS |
| | | EDTAVYYCARQQYDGRRYFGLWGRGTLVTVSS |
| 1104 | Anti-B7H4 | EIVMTQSPATLSVSPGERATLSCRASQSVSSNLAWYQQKPGQAP |
| | VL | RLLIYSASTRATGIPARFSGSGSGTEFTLTISSLQSEDFAVYYCQQ |
| | | VNVWPPTFGGGTKVEIK |
| 1105 | Anti-B7H4 | GTFSSYAIS |
| | CDR-H1 | |
| 1106 | Anti-B7H4 | GIIPIFGTANYAQKFQG |
| | CDR-H2 | |
| 1107 | Anti-B7H4 | ARGGPWFDP |
| | CDR-H3 | |
| 1108 | Anti-B7H4 | RASQSISSWLA |
| | CDR-L1 | |
| 1109 | Anti-B7H4 | KASSLES |
| | CDR-L2 | |
| 1110 | Anti-B7H4 | QQYNSYPPFT |
| | CDR-L3 | |
| 1111 | Anti-B7H4 | QVQLVQSGAEVKKPGSSVKVSCKASGGTFSSYAISWVRQAPGQ |
| | VH | GLEWMGGIIPIFGTANYAQKFQGRVTITADESTSTAYMELSSLRS |
| | | EDTAVYYCARGGPWFDPWGQGTLVTVSS |
| 1112 | Anti-B7H4 | DIQMTQSPSTLSASVGDRVTITCRASQSISSWLAWYQQKPGKAP |
| | VL | KLLIYKASSLESGVPSRFSGSGSGTEFTLTISSLQPDDFATYYCQQ |
| | | YNSYPPFTFGGGTKVEIK |
| 1113 | Anti-B7H4 | FTFSSYAMS |
| | CDR-H1 | |
| 1114 | Anti-B7H4 | AISGSGGSTSYADSVKG |
| | CDR-H2 | |
| 1115 | Anti-B7H4 | AKPSLATMLAFDI |
| | CDR-H3 | |
| 1116 | Anti-B7H4 | RASQSISSWLA |
| | CDR-L1 | |
| 1117 | Anti-B7H4 | DASSLES |
| | CDR-L2 | |
| 1118 | Anti-B7H4 | QQSKSYPRT |
| | CDR-L3 | |
| 1119 | Anti-B7H4 | EVQLLESGGGLVQPGGSLRLSCAASGFTFSSYAMSWVRQAPGK |
| | VH | GLEWVSAISGSGGSTSYADSVKGRFTISRDNSKNTLYLQMNSLR |
| | | AEDTAVYYCAKPSLATMLAFDIWGQGTMVTVSS |
| 1120 | Anti-B7H4 | DIQMTQSPSTLSASVGDRVTITCRASQSISSWLAWYQQKPGKAP |
| | VL | KLLIYDASSLESGVPSRFSGSGSGTEFTLTISSLQPDDFATYYCQQ |
| | | SKSYPRTFGGGTKVEIK |
| 1121 | Anti-B7H4 | GSISSSVYYWS |
| | CDR-H1 | |
| 1122 | Anti-B7H4 | SILVSGSTYYNPSLKS |
| | CDR-H2 | |
| 1123 | Anti-B7H4 | ARAVSFLDV |
| | CDR-H3 | |
| 1124 | Anti-B7H4 | RASQSISSYLN |
| | CDR-L1 | |
| 1125 | Anti-B7H4 | GASSLQS |
| | CDR-L2 | |
| 1126 | Anti-B7H4 | QQSYDPPWT |
| | CDR-L3 | |
| 1127 | Anti-B7H4 | QLQLQESGPGLVKPSETLSLTCTVSGGSISSSVYYWSWIRQPPGK |
| | VH | GLEWIGSILVSGSTYYNPSLKSRVTISVDTSKNQFSLKLSSVTAAD |
| | | TAVYYCARAVSFLDVWGQGTMVIVSS |
| 1128 | Anti-B7H4 | DIQMTQSPSSLSASVGDRVTITCRASQSISSYLNWYQQKPGKAPK |
| | VL | LLIYGASSLQSGVPSRFSGSGSGTDFTLTISSLQPEDFATYYCQQS |
| | | YDPPWTFGGGTKVEIK |
| 1129 | Anti-B7H4 | QLQLQESGPGLVKPSETLSLTCTVSGGSISSSSYYWGWIRQPPGK |
| | HC | GLEWIGNIYYSGSTYYNPSLKSRVTISVDTSKNQFSLKLSSVTAA |
| | | DTAVYYCAREGSYPNWFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1130 | Anti-B7H4 | EIVMTQSPATLSVSPGERATLSCRASQSVSSNLAWYQQKPGQAP |
| | LC | RLLIYGASTRATGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1131 | Anti-B7H4 | QLQLQESGPGLVKPSETLSLTCTVSGGSIKSGSHYWGWIRQPPGK |
| | HC | GLEWIGNIYYSGSTYYNPSLRSRVTISVDTSKNQFSLKLSSVTAA |
| | | DTAVYYCAREGSYPNWFDPWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1132 | Anti-B7H4 | EIVMTQSPATLSVSPGERATLSCRASQSVSSNLAWYQQKPGQAP |
| | LC | RLLIYGASTRATGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1133 | Anti-B7H4 | QLQLQESGPGLVKPSETLSLTCTVSGGSIKSGSHYWGWIRQPPGK |
| | HC | GLEWIGNIYYSGSTYYNPSLKSRVTISVDTSKNQFSLKLSSVTAA |
| | | DTAVYYCAREGSYPNWLDPWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1134 | Anti-B7H4 | EIVMTQSPATLSVSPGERATLSCRASQSVSSNLAWYQQKPGQAP |
| | LC | RLLIYGASTRATGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1135 | Anti-B7H4 | QLQLQESGPGLVKPSETLSLTCTVSGGSIKSGSYYWGWIRQPPGK |
| | HC | GLEWIGNIYYSGSTYYNPSLKSRVTISVDTSKNQFSLKLSSVTAA |
| | | DTAVYYCAREGSYPNQFDPWGQGILVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1136 | Anti-B7H4 | EIVMTQSPATLSVSPGERATLSCRASQSVSSNLAWYQQKPGQAP |
| | LC | RLLIYGASTRATGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1137 | Anti-B7H4 | QLQLQESGPGLVKPSETLSLTCTVSGGSIKSGSHYWGWIRQPPGK |
| | HC | GLEWIGNIYYSGSTYYNPSLKSRVTMSVDTSKNQFSLKLSSVTA |
| | | ADTAVYYCAREGSYPNWFDPWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1138 | Anti-B7H4 | EIVMTQSPATLSVSPGERATLSCRASQSVSTNLAWYQQKPGQAP |
| | LC | RLLIYDASARVTGIPARFSGSGSGTEFTLTISSLQSEDFAVYYCQQ |
| | | YHSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1139 | Anti-B7H4 | QLQLQESGPGLVKPSETLSLTCTVSGGSISSSSYYWGWIRQPPGK |
| | HC | GLEWIGNIYYSGSTYYNPSLKSRVTISVDTSKNQFSLKLSSVTAA |
| | | DTAVYYCAREGSYTTVLNVWGQGTMVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1140 | Anti-B7H4 | EIVLTQSPGTLSLSPGERATLSCRASQSVSSSYLAWYQQKPGQAP |
| | LC | RLLIYGASSRATGIPDRFSGSGSGTDFTLTISRLEPEDFAVYYCQQ |
| | | AASYPLTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1141 | Anti-B7H4 | QLQLQESGPGLVKPSETLSLTCTVSGGSIGRGSYYWGWIRQPPG |
| | HC | KGLEWIGNIYYSGSTYYNPSLKSRVTISVDTSKNQFSLKLSSVTA |
| | | ADTAVYYCAREGSYTTVLNVWGQGTMVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1142 | Anti-B7H4 | EIVLTQSPGTLSLSPGERATLSCRASQSVASSHLAWYQQKPGQAP |
| | LC | RLLIYDAVSRATGIPDRFSGSGSGTDFTLTISRLEPEDFAVYYCQQ |
| | | AASYPLTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1143 | Anti-B7H4 | QVQLQESGPGLVKPSQTLSLTCTVSGGSISSGGYYWSWIRQHPG |
| | HC | KGLEWIGNIYYSGSTYYNPSLKSRVTISVDTSKNQFSLKLSSVTA |
| | | ADTAVYYCARESSTISADFDLWGRGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1144 | Anti-B7H4 | DIQMTQSPSSVSASVGDRVTITCRASQGISRWLAWYQQKPGKAP |
| | LC | KLLIYAASSLQSGVPSRFSGSGSGTDFTLTISSLQPEDFATYYCQQ |
| | | AHTFPYTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1145 | Anti-B7H4 | QLQLQESGPGLVKPSETLSLTCTASGGSISHGGYYWSWIRQHPG |
| | HC | KGLEWIGNIYYSGSTYYNPSLKSRVTMSVDTSKNQFSLKLSSVT |
| | | AADTAVYYCARESSTISADFDLWGRGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVEP |
| | | KSCDKTHTCPPCPAPELLGGPSVFLFPPKPKDTLMISRTPEVTCVV |
| | | VDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 1146 | Anti-B7H4 | DIQMTQSPSSVSASVGDRVTITCRASQGISRWLAWYQQKPGKAP |
| | LC | KLLIYAASSLQSGVPSRFSGSGSGTDFTLTISSLQPEDFATYYCQQ |
| | | AHTFPYTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1147 | Anti-B7H4 | QVQLQESGPGLVKPSQTLSLTCTVSGGSISSGGYYWSWIRQHPG |
| | HC | KGLEWIGNIYYSGSTYYNPSLKSRVTISVDTSKNQFSLKLSSVTA |
| | | ADTAVYYCARGLSTIDEAFDPWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1148 | Anti-B7H4 | DIQMTQSPSTLSASVGDRVTITCRASQSISSWLAWYQQKPGKAP |
| | LC | KLLIYKASSLESGVPSRFSGSGSGTEFTLTISSLQPDDFATYYCQQ |
| | | DNSYPYTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1149 | Anti-B7H4 | QLQLQESGPGLVKPSETLSLTCTVSGGSISDGSYYWSWIRQHPGK |
| | HC | GLEWIGNIYYSGSTYYNPSLRSRVTMSVDTSKNQFSLKLSSVTAA |
| | | DTAVYYCARGLSTIDEAFDPWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1150 | Anti-B7H4 | DIQMTQSPSTLSASVGDRVTITCRASQSISSWLAWYQQKPGKAP |
| | LC | KLLIYKASSLESGVPSRFSGSGSGTEFTLTISSLQPDDFATYYCQQ |
| | | DNSYPYTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1151 | Anti-B7H4 | QVQLQESGPGLVKPSQTLSLTCTVSGGSISDGSYYWSWIRQHPG |
| | HC | KGLEWIGNIYYSGSTYYNPSLRSRVTMSVDTSKNQFSLKLSSVTA |
| | | ADTAVYYCARGLSTIDEAFDPWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1152 | Anti-B7H4 | DIQMTQSPSTLSASVGDRVTITCRASKSISSWLAWYQQKPGKAP |
| | LC | KLLIYEASSLHSGVPSRFSGSGSGTEFTLTISSLQPDDFATYYCQQ |
| | | DNSYPYTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1153 | Anti-B7H4 | QVQLQESGPGLVKPSETLSLTCTVSGGSISSYYWSWIRQPPGKGL |
| | HC | EWIGYIYSSGSTNYNPSLKSRVTISVDTSKNQFSLKLSSVTAADT |
| | | AVYYCARGSGQYAAPDYGMDVWGQGTTVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 1154 | Anti-B7H4 | DIQMTQSPSTLSASVGDRVTITCRASQSISSWLAWYQQKPGKAP |
| | LC | KLLIYKASSLESGVPSRFSGSGSGTEFTLTISSLQPDDFATYYCQQ |
| | | DNSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1155 | Anti-B7H4 | QVQLQESGPGLVKPSETLSLTCTVSGGSIISYYWGWIRQPPGKGL |
| | HC | EWIGYIYSSGSTSYNPSLKSRVTISVDTSKNQFSLKLSSVTAADTA |
| | | VYYCARGSGLYAAPDYGLDVWGQGTTVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1156 | Anti-B7H4 | DIQMTQSPSTLSASVGDRVTITCRASQSISSWLAWYQQKPGKAP |
| | LC | KLLIYKASSLESGVPSRFSGSGSGTEFTLTISSLQPDDFATYYCQQ |
| | | DNSFPFTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1157 | Anti-B7H4 | EVQLLESGGGLVQPGGSLRLSCAASGFTFSSYAMSWVRQAPGK |
| | HC | GLEWVSTISGSGGSTYYADSVKGRFTISRDNSKNTLYLQMNSLR |
| | | AEDTAVYYCARGAGHYDLVGRYWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 1158 | Anti-B7H4 | DIQMTQSPSSLSASVGDRVTITCRASQSISSYLNWYQQKPGKAPK |
| | LC | LLIYAASSLQSGVPSRFSGSGSGTDFTLTISSLQPEDFATYYCQQL |
| | | YSLPPTFGGGTKVEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| 1159 | Anti-B7H4 | EVQLLESGGGLVQPGGSLRLSCAASGFTFSSYAMSWVRQAPGK |
| | HC | GLEWVSAISGSGGSTYYADSVKGRFTISRDNSKNTLYLQMNSLR |
| | | AEDTAVYYCARVGFRALNYWGQGTTVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLT |
| | | VLHQDWLNGKEYKCKVSNKALPAPIEKTISKAKGQPREPQVYTL |
| | | PPSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 1160 | Anti-B7H4 | DIQLTQSPSSVSASVGDRVTITCRASQDISSWLAWYQQKPGKAP |
| | LC | KLLIYAASSLQSGVPSRFSGSGSGTDFTLTISSLQPEDFATYYCQQ |
| | | ATSYPPWTFGGGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC |
| 1161 | Anti-B7H4 | QVQLVQSGAEVKKPGSSVKVSCKASGGTFSSYAISWVRQAPGQ |
| | HC | GLEWMGGIIPIFGTASYAQKFQGRVTITADESTSTAYMELSSLRS |
| | | EDTAVYYCARQQYDGRRYFGLWGRGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVEP |
| | | KSCDKTHTCPPCPAPELLGGPSVFLFPPKPKDTLMISRTPEVTCVV |
| | | VDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 1162 | Anti-B7H4 | EIVMTQSPATLSVSPGERATLSCRASQSVSSNLAWYQQKPGQAP |
| | LC | RLLIYSASTRATGIPARFSGSGSGTEFTLTISSLQSEDFAVYYCQQ |
| | | VNVWPPTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1163 | Anti-B7H4 | QVQLVQSGAEVKKPGSSVKVSCKASGGTFSSYAISWVRQAPGQ |
| | HC | GLEWMGGIIPIFGTANYAQKFQGRVTITADESTSTAYMELSSLRS |
| | | EDTAVYYCARGGPWFDPWGQGTLVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPGK |
| 1164 | Anti-B7H4 | DIQMTQSPSTLSASVGDRVTITCRASQSISSWLAWYQQKPGKAP |
| | LC | KLLIYKASSLESGVPSRFSGSGSGTEFTLTISSLQPDDFATYYCQQ |
| | | YNSYPPFTFGGGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 1165 | Anti-B7H4 | EVQLLESGGGLVQPGGSLRLSCAASGFTFSSYAMSWVRQAPGK |
| | HC | GLEWVSAISGSGGSTSYADSVKGRFTISRDNSKNTLYLQMNSLR |
| | | AEDTAVYYCAKPSLATMLAFDIWGQGTMVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 1166 | Anti-B7H4 | DIQMTQSPSTLSASVGDRVTITCRASQSISSWLAWYQQKPGKAP |
| | LC | KLLIYDASSLESGVPSRFSGSGSGTEFTLTISSLQPDDFATYYCQQ |
| | | SKSYPRTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1167 | Anti-B7H4 | QLQLQESGPGLVKPSETLSLTCTVSGGSISSSVYYWSWIRQPPGK |
| | HC | GLEWIGSILVSGSTYYNPSLKSRVTISVDTSKNQFSLKLSSVTAAD |
| | | TAVYYCARAVSFLDVWGQGTMVIVSSASTKGPSVFPLAPSSKST |
| | | SGGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | RDELTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 1168 | Anti-B7H4 | DIQMTQSPSSLSASVGDRVTITCRASQSISSYLNWYQQKPGKAPK |
| | LC | LLIYGASSLQSGVPSRFSGSGSGTDFTLTISSLQPEDFATYYCQQS |
| | | YDPPWTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1169 | Anti-CD70 | NYGMN |
| | CDR-H1 | |
| 1170 | Anti- | WINTYTGEPTYADAFKG |
| | CD70CDR- | |
| | H2 | |
| 1171 | Anti-CD70 | DYGDYGMDY |
| | CDR-H3 | |
| 1172 | Anti-CD70 | RASKSVSTSGYSFMH |
| | CDR-L1 | |
| 1173 | Anti-CD70 | LASNLES |
| | CDR-L2 | |
| 1174 | Anti-CD70 | QHSREVPWT |
| | CDR-L3 | |
| 1175 | Anti-CD19 | QVQLQESGPGLVKPSQTLSLTCTVSGGSISTSGMGVGWIRQHPG |
| | (hBU12) HC | KGLEWIGHIWWDDDKRYNPALKSRVTISVDTSKNQFSLKLSSVT |
| | | AADTAVYYCARMELWSYYFDYWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVEP |
| | | KSCDKTHTCPPCPAPELLGGPSVFLFPPKPKDTLMISRTPEVTCVV |
| | | VDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 1176 | Anti-CD19 | EIVLTQSPATLSLSPGERATLSCSASSSVSYMHWYQQKPGQAPRL |
| | (hBU12) LC | LIYDTSKLASGIPARFSGSGSGTDFTLTISSLEPEDVAVYYCFQGS |
| | | VYPFTFGQGTKLEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| 1177 | Anti-ZIP6 | QVQLVQSGAEVKKPGASVKVSCKASGLTIEDYYMHWVRQAPG |
| | HC | QGLEWMGWIDPENGDTEYGPKFQGRVTMTRDTSINTAYMELSR |
| | | LRSDDTAVYYCAVHNAHYGTWFAYWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEV |
| | | TCVVVDVSHEDPEVKFNWYVDGVEVHNAKTKPREEQYNSTYR |
| | | VVSVLTVLHQDWLNGKEYKCKVSNKALPAPIEKTISKAKGQPRE |
| | | PQVYTLPPSRDELTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 1178 | Anti-ZIP6 | DVVMTQSPLSLPVTLGQPASISCRSSQSLLHSSGNTYLEWYQQRP |
| | LC | GQSPRPLIYKISTRFSGVPDRFSGSGSGTDFTLKISRVEAEDVGVY |
| | | YCFQGSHVPYTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC |
| 1179 | Anti-gpNMB | QVQLQESGPGLVKPSQTLSLTCTVSGGSISSFNYYWSWIRHHPGK |
| | HC | GLEWIGYIYYSGSTYSNPSLKSRVTISVDTSKNQFSLTLSSVTAAD |
| | | TAVYYCARGYNWNYFDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALPAPIEKTISKAKGQPREPQVYTLPP |
| | | SRDELTKNQVSLTCLVKGFYPSDIAVEWESNGQPENNYKTTPPV |
| | | LDSDGSFFLYSKLTVDKSRWQQGNVFSCSVMHEALHNHYTQKS |
| | | LSLSPGK |
| 1180 | Anti-gpNMB | EIVMTQSPATLSVSPGERATLSCRASQSVDNNLVWYQQKPGQAP |
| | LC | RLLIYGASTRATGIPARFSGSGSGTEFTLTISSLQSEDFAVYYCQQ |
| | | YNNWPPWTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 1181 | Anti- | DMGWGSGWRPYYYYGMDV |
| | EpCAM | |
| | CDR-H3 | |
| 1182 | Anti- | QSVLTQPPSVSGAPGQRVTISCTGSSSNIGSYYGVHWYQQLPGTA |
| | EpCAM VL | PKLLIYSDTNRPSGVPDRFSGSKSGTSASLAITGLQAEDEADYYC |
| | | QSYDKGFGHRVFGGGTKLTVL |
| 1183 | Anti- | SYWMH |
| | ADAM9 | |
| | CDR-H1 | |
| 1184 | Anti- | SYWMH |
| | ADAM9 | |
| | CDR-H1 | |
| 1185 | Anti- | KASQSVDYSGDSYMN |
| | ADAM9 | |
| | CDR-L1 | |
| 1186 | Anti-CD59 | SYGMN |
| | CDR-H1 | |
| 1187 | Anti-CD59 | YISSSSSTIYYADSVKG |
| | CDR-H2 | |
| 1188 | Anti-CD59 | QVQLQQSGGGVVQPGRSLGLSCAASGFTFSSYGMNWVRQAPGK |
| | VH | GLEWVSYISSSSSTIYYADSVKGRFTISRDNSKNTLYLQMNSLRA |
| | | EDTAVYYCARGPGMDVWGQGTTVTVS |
| 1189 | Anti-B7-H3 | YISSDSSAIYYADTVKG |
| | CDR-H2 | |
| 1190 | Anti-B7-H3 | GRENIYYGSRLDY |
| | CDR-H3 | |
| 1191 | Anti-B7-H3 | KASQNVDTNVA |
| | CDR-L1 | |
| 1192 | Anti-B7-H3 | SASYRYS |
| | CDR-L2 | |
| 1193 | Anti-B7-H3 | QQYNNYPFT |
| | CDR-L3 | |
| 1194 | Anti-B7-H3 | SYGMS |
| | CDR-H1 | |
| 1195 | Anti-B7-H3 | TINSGGSNTYYPDSLKG |
| | CDR-H2 | |
| 1196 | Anti-B7-H3 | HDGGAMDY |
| | CDR-H3 | |
| 1197 | Anti-B7-H3 | RASESIYSYLA |
| | CDR-L1 | |
| 1198 | Anti-B7-H3 | QHHYGTPPWT |
| | CDR-L3 | |
| 1199 | Anti-B7-H3 | SFGMH |
| | CDR-H1 | |
| 1200 | Anti-B7-H3 | YISSGSGTIYYADTVKG |
| | CDR-H2 | |
| 1201 | Anti-B7-H3 | HGYRYEGFDY |
| | CDR-H3 | |
| 1202 | Anti-B7-H3 | KASQNVDTNVA |
| | CDR-L1 | |
| 1203 | Anti-B7-H3 | SASYRYS |
| | CDR-L2 | |
| 1204 | Anti-B7-H3 | QQYNNYPFT |
| | CDR-L3 | |
| 1205 | Anti-B7-H3 | SYTIH |
| | CDR-H1 | |
| 1206 | Anti-B7-H3 | DIQLTQSPSFLSASVGDRVTITCRASSSVSYMNWYQQKPGKSPKP |
| | VL | WIYATSNLASGVPSRFSVSVSGTEHTLTISSLQPEDFATYYCQQW |
| | | SSNPLTFGQGTKLEIK |
| 1207 | Anti-B7-H3 | SYWMH |
| | CDR-H1 | |
| 1208 | Anti-B7-H3 | GGRLYFDY |
| | CDR-H3 | |
| 1209 | Anti-B7-H3 | SYWMH |
| | CDR-H1 | |
| 1210 | Anti-B7-H3 | DIVMTQSPLSLPVTPGEPASISCRSSQSLVHSNQDTYLRWYLQKP |
| | VL | GQSPQLLIYKVSNRFSGVPDRFSGSGSGTDFTLKISRVEAEDVGV |
| | | YYCSQSTHVPYTFGGGTKVEIK |
| 1211 | Anti-B7-H3 | SGYSWH |
| | CDR-H1 | |
| 1212 | Anti-B7-H3 | DIQMTQSPSSLSASVGDRVTITCKASQNVGFNVAWYQQKPGKSP |
| | VL | KALIYSASYRYSGVPSRFSGSGSGTDFTLTISSLQPEDFAEYFCQQ |
| | | YNWYPFTFGQGTKLEIK |
| 1213 | Anti-B7-H3 | WIFPGDDSTQYNEKFKG |
| | CDR-H2 | |
| 1214 | Anti-B7-H3 | QNGHSFPLT |
| | CDR-L3 | |
| 1215 | Anti-CDCP1 | DIQMTQSPSSLSASVGDRVTITCSVSSSVFYVHWYQQKPGKAPK |
| | VL | LLIYDTSKLASGVPSRFSGSGSGTDFTFTISSLQPEDIATYYCQQW |
| | | NSNPPTFGGGTKVEIK |
| 1216 | Anti-CDCP1 | QIVLTQSPAIMSASPGEKVTMTCSVSSSVFYVHWYQQKSGTSPK |
| | VL | RWIYDTSKLASGVPARFSGSGSGTSYSLTISSMEAEDAATYYCQ |
| | | QWNSNPPTFGGGTKLEIK |
| 1217 | Anti-CDCP1 | DGVLRYFDWLLDYYYYMDV |
| | CDR-H3 | |
| 1218 | Anti-GPC3 | GIDPETGGTAYNQKFKG |
| | CDR-H2 | |
| 1219 | Anti-GPC3 | RSSQSIVHSNANTYLQ |
| | CDR-L1 | |
| 1220 | Anti-TIGIT | GPSEVGAILGYVWFDP |
| | CDR-H3 | |
| 1221 | Anti-CD33 | DIQMTQSPSSLSASVGDRVTINCKASQDINSYLSWFQQKPGKAPK |
| | VL | TLIYRANRVDGVPSRFSGSGSGQDYTLT |
| | | ISSLQPEDFATYYCLQYDEFPLTFGGGTKVEIK |

1. A Camptothecin Conjugate having the formula of
L-(Q-D)p
or a salt thereof, wherein
L is a Ligand Unit from a targeting agent, in particular from an antibody that selectively binds to a cancer cell antigen;
subscript p is an integer ranging from 1 to 16;
Q is a Linker Unit having a formula selected from the group consisting of:
-Z-A-, -Z-A-RL-, -Z-A-RL-Y-, -Z-A-S*-RL-, -Z-A-S*-RL-Y-, -Z-A-S*-W-, -Z-A-S*-W-RL-, -Z-A-B(S*)-RL-, -Z-A-B(S*)-W-, -Z-A-B(S*)-W-RL- and -Z-A-B(S*)-RL-Y-,
wherein Z is a Stretcher Unit;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit D having a formula of D_{0b} 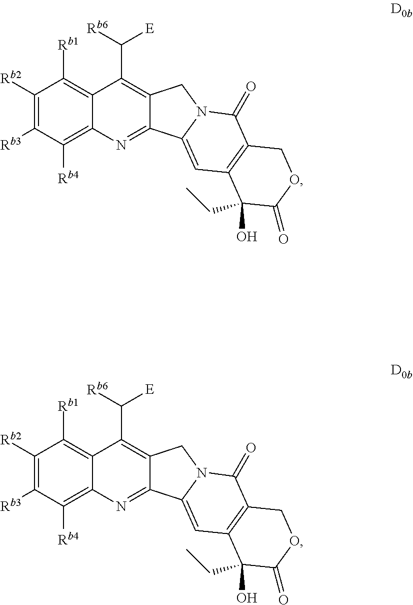
or a salt thereof, wherein;
E is —OR^{b5} or —NR^{b5}R^{b5};
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁⁻C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with —OR^{a}, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form a 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₈ alkyl-O-C₁-C₈ alkyl-, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, C₁-C₆ hydroxyalkyl-heteroaryl-, phenyl, phenyl-C₁-C₄ alkyl-, diphenyl-C₁-C₄ alkyl-, heteroaryl, heteroaryl-C₁-C₄ alkyl-, C₁-C₆ alkoxy-C(O)—C₁-C₈ aminoalkyl-, C₁-C₆ alkoxy-C(O)-N—(C₁-C₄ alkyl)amino-C₁-C₈ alkyl-, C₁-C₆ alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂—C₁-C₈ alkyl-, (C₃-C₁₀heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane and E is —NR^{b5}R^{b5}′, then each R^{b5} and R^{b5}′ are independently selected from the group consisting of H, C₁-C₈ alkyl-O-C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆alkoxy-C(O)—C₁-C₈ aminoalkyl-, C₁-C₆ alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂—C₁-C₈ alkyl-, NH₂—SO₂-C₁-C₈ alkyl-, (C₃-C₁₀ heterocycloalkyl)-C₁-C₄ hydroxyalkyl-, C₁-C₆ alkoxy-C(O)—(C₃-C to heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
b) when R^{b2} is combined with R^{b3} and the intervening atoms to form a 1,3-dioxolane, E is not —OH; and
c) when R^{b2} is methyl and R^{b3} is F, then R^{b1} does not come together with R^{b6} and the intervening atoms to form a ring, and
d) D is not (S)-7-ethyl-7-hydroxy-14-((4-methylpiperazin-1-yl)methyl)-10,13-dihydro-11H-[1,3]dioxolo[4,5-g]pyrano[3′,4′:6,7]indolizino[1,2-b]quinoline-8,11(7H)-dione or (S)-7-ethyl-7-hydroxy-14-(morpholinomethyl)-10,13-dihydro-11 H-[1,3]dioxolo[4,5-g]pyrano[3′,4′:6,7]indolizino[1,2-b]quinoline-8,11(7H)-dione, or a salt of any of the foregoing;
wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

2. The Camptothecin Conjugate of claim 1, wherein D has a formula selected from the group consisting of 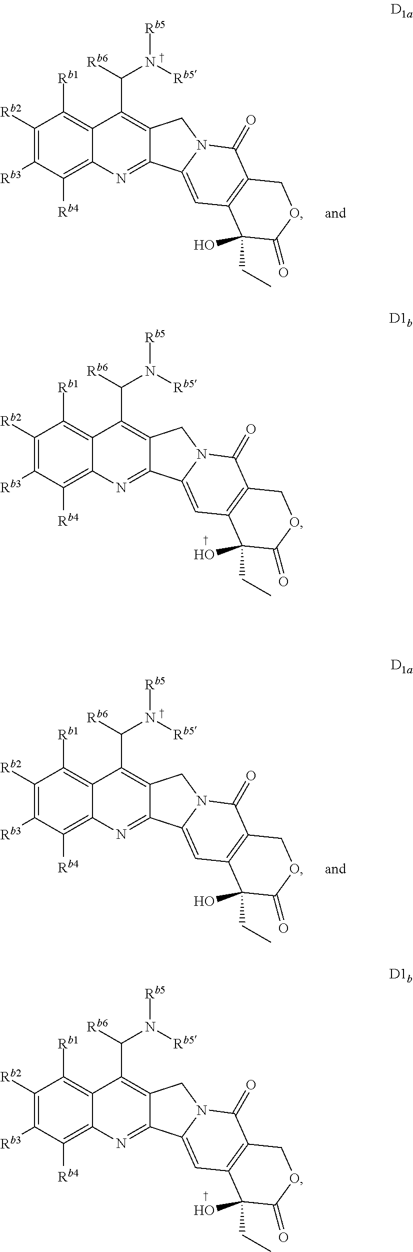
or a salt thereof, wherein the dagger indicates the site of covalent attachment of D to Q, or wherein a hydrogen atom of a hydroxyl, thiol, primary amine, or secondary amine of R^{b5} is replaced with a bond to Q or a tertiary amine of R^{b5} is quaternized to form a bond to Q.

3. (canceled)

4. The Camptothecin Conjugate of claim 2, wherein D has a formula selected from the group consisting of 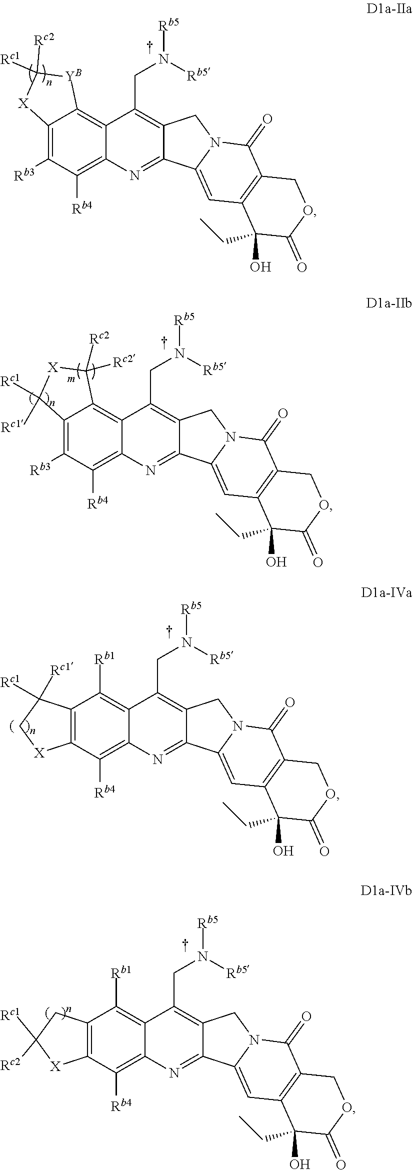 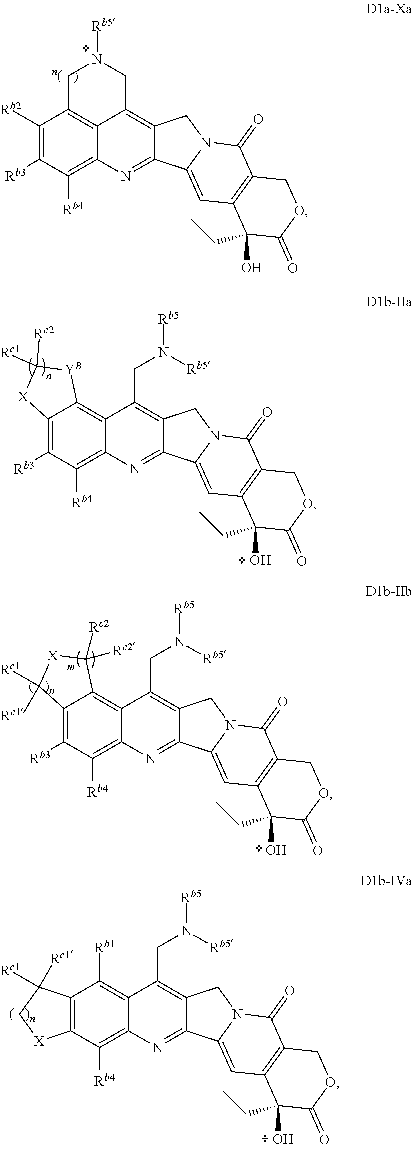 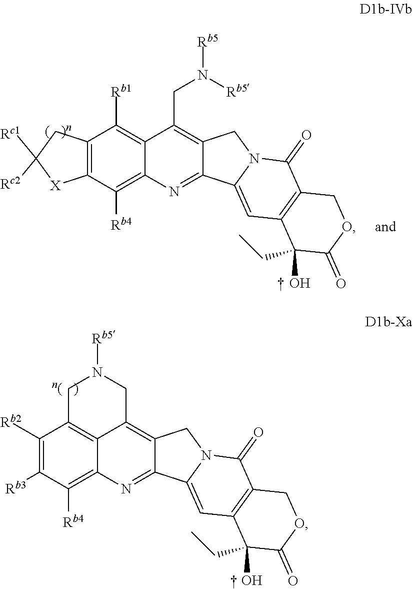
or a salt thereof, wherein
X and Y^{B} are each independently 0, S, S(O)₂, CR^{x}R^{x}′, or NR^{x};
R^{x} and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently
(i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}′, and —SR^{a}, C₁-C₆ alkyl-C(O)—, C₁-C₆ alkyl-NR^{a}—C(O)—, and C₁-C₆ alkyl-S(O)₂—; or
(ii) taken together with R^{b1} and the intervening atoms to form a 5- to 7-membered carbocyclo or heterocyclo; or
(iii) taken together with R^{x}′ and the intervening atoms to form a 3 to 6-membered carbocyclo or heterocyclo; and
when m and n are both present, the sum of m+n is 2 or 3;
wherein the dagger indicates the site of covalent attachment of D to Q, or wherein a hydrogen atom of a hydroxyl, thiol, primary amine, or secondary amine of R^{b5} is replaced with a bond to Q or a tertiary amine of R^{b5} is quaternized to form a bond to Q.

5-16. (canceled)

17. A Camptothecin Conjugate having the formula of
L-(Q-D)ₚ
or a salt thereof, wherein
L is a Ligand Unit from a targeting agent, in particular from an antibody that selectively binds to a cancer cell antigen;
subscript p is an integer ranging from 1 to 16;
Q is a Linker Unit having a formula selected from the group consisting of:
Z-A-, -Z-A-RL-, -Z-A-RL-Y-, -Z-A-S*-RL-, -Z-A-S*-RL-Y-, -Z-A-S*-W-, -Z-A-S*-W-RL-, -Z-A-B(S*)-RL-, -Z-A-B(S*)-W-, -Z-A-B(S*)-W-RL- and -Z-A-B(S*)-RL-Y-,
wherein Z is a Stretcher Unit;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit D, wherein D comprises a compound selected from Table I, or a salt thereof, wherein D is covalently attached to Q via any suitable attachment site on D, optionally wherein a hydrogen atom of a hydroxyl, thiol, primary amine, or secondary amine of D is replaced with a bond to Q or a tertiary amine of D is quaternized to form a bond to Q.

18. The Camptothecin Conjugate of claim 1, wherein Q is a Linker Unit having the formula selected from the group consisting of:
-Z-A-RL-; -Z-A-RL-Y-; -Z-A-S*-RL-; -Z-A-B(S*)-RL-; -Z-A- S*-RL-Y-; and -Z-A-B(S*)-RL-Y-,
wherein A is a Connector Unit and RL is a Glycoside (e.g., Glucuronide) Unit.

19. The Camptothecin Conjugate of claim 18, wherein the Glycoside (e.g., Glucuronide) Unit has the formula of: 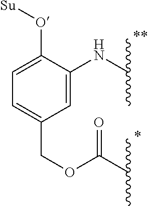
wherein
Su is a hexose form of a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q,
optionally wherein the Glycoside (e.g., Glucuronide) Unit has the formula of: 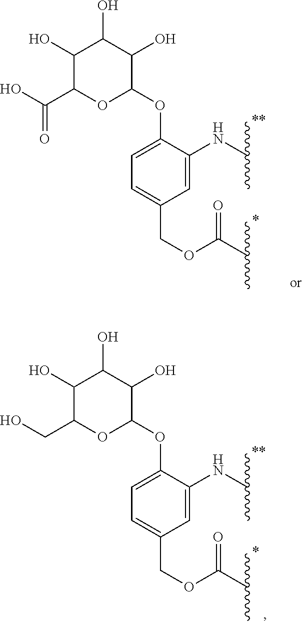 [SMILES: *Nc1cc(COC(*)=O)ccc1OC1OC(C(=O)O)C(O)C(O)C1O ; *Nc1cc(COC(*)=O)ccc1OC1OC(CO)C(O)C(O)C1O]
or optionally wherein the Glycoside (e.g., Glucuronide) Unit has the formula of:

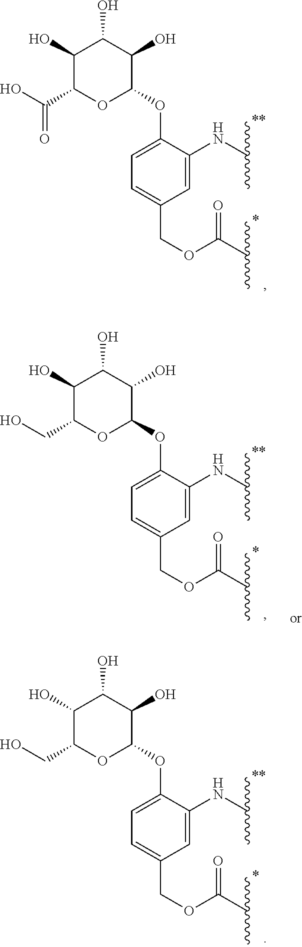 [SMILES: *Nc1cc(COC(*)=O)ccc1O[C@@H]1O[C@H](C(=O)O)[C@@H](O)[C@H](O)[C@H]1O ; *Nc1cc(COC(*)=O)ccc1O[C@H]1O[C@H](CO)[C@@H](O)[C@H](O)[C@@H]1O ; *Nc1cc(COC(*)=O)ccc1O[C@@H]1O[C@H](CO)[C@H](O)[C@H](O)[C@H]1O]

20-30. (canceled)

31. A Camptothecin-Linker compound having a formula selected from the group consisting of:
(i) Z′-A-RL-D;
(ii) Z′-A-RL-Y-D;
(iii) Z′-A-S*-RL-D;
(iv) Z′-A-S*-RL-Y-D;
(v) Z′-A-B(S*)-RL-D;
(vi) Z′-A-B(S*)-RL-Y-D;
(vii) Z′-A-D
(viii) Z′-A-S*-W-D
(ix) Z′-A-B(S*)-W-D
(x) Z′-A-S*-W-RL-D; and
(xi) Z′-A-B(S*)-W-RL-D
wherein
Z′ is a Stretcher Unit precursor;
A is a bond or a Connector Unit;
B is a Parallel Connector Unit;
S* is a Partitioning Agent;
RL is a Releasable Linker;
Y is a Spacer Unit; and
D is a Drug Unit D having a formula of has a formula of D_{0b} 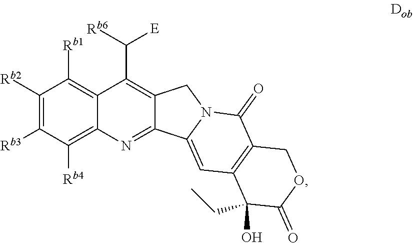
or a salt thereof; wherein;
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR′, —NR^{a}R^{a}, and —SR^{a}; each optionally substituted with C₁-C₃ alkyl, —OR′, —NR^{a}R^{a}, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂-, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR′, —NR^{a}R^{a}′, and —SR^{a}; each optionally substituted with —OR′, —NR^{a}R^{a}′, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}′, and —SR^{a};
R^{b4} is selected from the group consisting of H or halogen;
each R^{b5} and R^{b5}′; are independently selected from the group consisting of H, C₁-C₈ alkyl, C₁-C₈ hydroxyalkyl, C₁-C₈ alkyl-O-C₁-C₈ alkyl-, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, C₁-C₆ hydroxyalkyl-heteroaryl-, phenyl, phenyl-C₁-C₄ alkyl-, diphenyl-C₁-C₄ alkyl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆ alkoxy-C(O)—(C₃-C₁₀ heterocycloalkyl)-C₁-C₅ alkyl-, C₁-C₄ alkyl-SO₂-C₁-C₈ alkyl-, NH₂—SO₂-C₁-C₈ alkyl-, (C₃-C₁₀heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′; are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1}and the intervening atoms to form a carbocyclo or heterocyclo; and
R^{a} and R^{a}′ are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane and E is —NR^{b5}R^{b5}, then each R^{b5} and R^{b5}′; are independently selected from the group consisting of H, C₁-C₈ alkyl-O-C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆ alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆ alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆ alkoxy-C(O)—(C₃-C₁₀ heterocycloalkyl)-C₁-C₅ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆ alkoxy-C(O)—C₁-C₈ aminoalkyl-, and C₁-C₈ aminoalkyl; or
R^{b5} is H and R^{b5}′ is combined with R^{b1}and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
b) when R^{b2} is combined with R^{b3} and the intervening atoms to form a 1,3-dioxolane, E is not —OH; and
c) when R^{b2} is methyl and R^{b3} is F, then R^{b1} does not come together with R^{b6} and the intervening atoms to form a ring, and
d) D is not (S)-7-ethyl-7-hydroxy-14-((4-methylpiperazin-1-yl)methyl)-10,13-dihydro-11 H-[1,3]dioxolo[4,5-g]pyrano[3′,4′:6,7]indolizino[1,2-b]quinoline-8,11(7H)-dione or (S)-7-ethyl-7-hydroxy-14-(morpholinomethyl)-10,13-dihydro-11H-[1,3]dioxolo[4,5-g]pyrano[3′,4′:6,7]indolizino[1,2-b]quinoline-8,11(7H)-dione, or a salt of any of the foregoing;
wherein D is covalently attached to the remainder of the Camptothecin-Linker compound via any suitable attachment site on D, optionally wherein a hydrogen atom of a hydroxyl, thiol, primary amine, or secondary amine of D is replaced with a bond to the remainder of the Camptothecin-Linker compound or a tertiary amine of D is quaternized to form a bond to the remainder of the Camptothecin-Linker compound.

32. The Camptothecin-Linker compound of claim 31, wherein D has a formula selected from the group consisting of 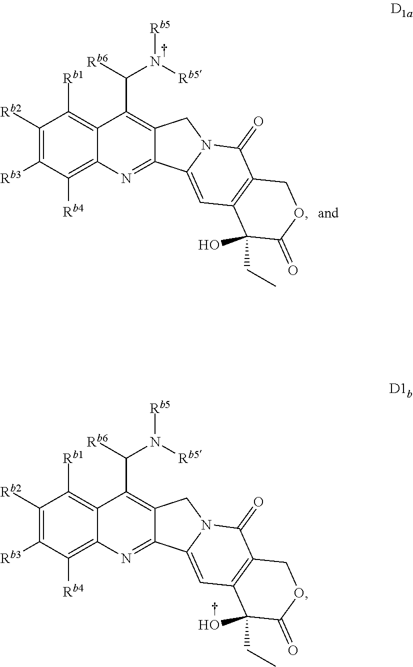
or a salt thereof, wherein the dagger indicates the site of covalent attachment of D to the remainder of the Camptothecin-Linker compound, or wherein a hydrogen atom of a hydroxyl, thiol, primary amine, or secondary amine of R^{b5} is replaced with a bond to the remainder of the Camptothecin-Linker compound or a tertiary amine of R^{b5} is quaternized to form a bond to the remainder of the Camptothecin-Linker compound.

33. (canceled)

34. The Camptothecin-Linker compound of claim 32, wherein D has a formula selected from the group consisting of 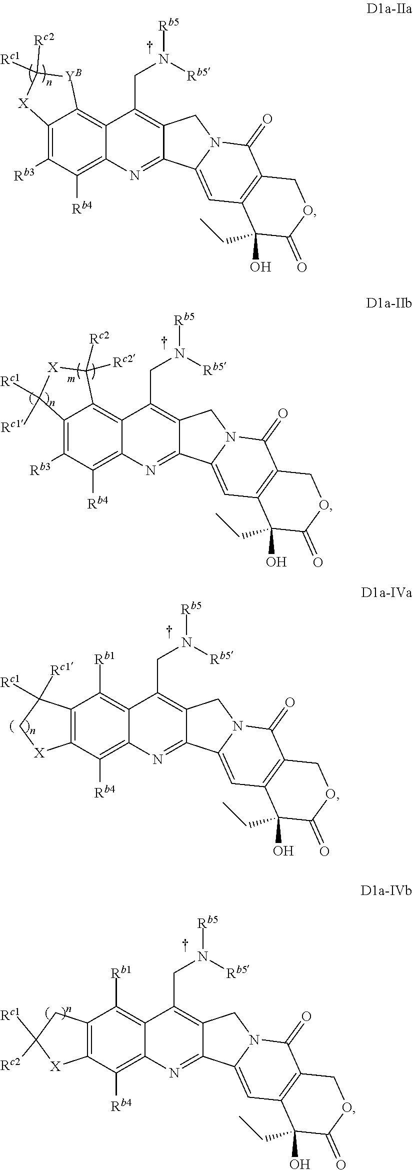 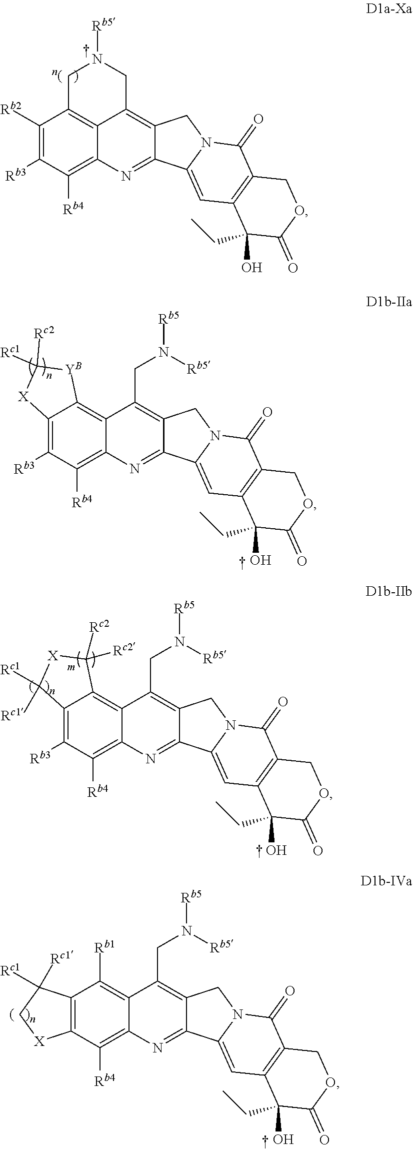 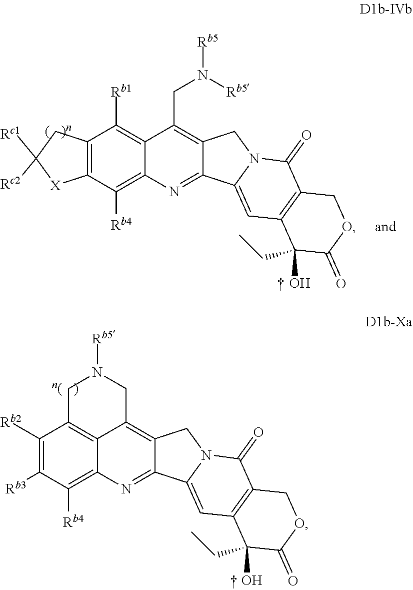
or a salt thereof, wherein
X and Y^{B} are each independently O, S, S(O)₂, CR^{x}R^{x}′, or NRx;
R^{x}and R^{x}′ are each independently selected from the group consisting of H, OH, C₁-C₆ alkyl, C₁-C₆ haloalkyl, C₁-C₆ aminoalkyl-C(O)—, C₁-C₆ alkyl-C(O)—, C₁-C₆ hydroxyalkyl-C(O)—, C₁-C₆ alkyl-NH—C(O)—, or C₁-C₆ alkyl-S(O)₂—; and
m and n are each 1 or 2;
each R^{c1}, R^{c1}′, R^{c2}, and R^{c2}′ is independently
(i) selected from the group consisting of H, halogen, C₁-C₆ alkyl, C₁-C₆ haloalkyl, C₁-C₆ hydroxyalkyl, C₁-C₆ aminoalkyl, —OR^{a}, —NR^{a}R^{a}′, and —SR^{a}, C₁-C₆ alkyl-C(O)—, C₁-C₆ alkyl-NR^{a}—C(O)—, and C₁-C₆ alkyl-S(O)₂—; or
(ii) taken together with R^{b1} and the intervening atoms to form a 5- to 7-membered carbocyclo or heterocyclo; or
(iii) taken together with R^{x}′ and the intervening atoms to form a 3 to 6-membered carbocyclo or heterocyclo; and
when m and n are both present, the sum of m+n is 2 or 3;
wherein the dagger indicates the site of covalent attachment of D to the remainder of the Camptothecin-Linker compound, or wherein a hydrogen atom of a hydroxyl, thiol, primary amine, or secondary amine of R^{b5} is replaced with a bond to the remainder of the Camptothecin-Linker compound or a tertiary amine of R^{b5} is quaternized to form a bond to the remainder of the Camptothecin-Linker compound.

35-45. (canceled)

46. A Camptothecin-Linker compound the formula of
(i) Z′-A-RL-D;
(ii) Z′-A-RL-Y-D;
(iii) Z′-A-S*-RL-D;
(iv) Z′-A-S*-RL-Y-D;
(v) Z′-A-B(S*)-RL-D;
(vi) Z′-A-B(S*)-RL-Y-D;
(vii) Z′-A-D
(viii) Z′-A-S*-W-D
(ix) Z′-A-B(S*)-W-D
(x) Z′-A-S*-W-RL-D; and
(xi) Z′-A-B(S*)-W-RL-D
wherein
Z′ is a Stretcher Unit precursor;
A is a bond or a Connector Unit;
B is a Parallel Connector Unit;
S* is a Partitioning Agent;
RL is a Releasable Linker;
Y is a Spacer Unit; and
D is a Drug Unit D, wherein D comprises a compound selected from Table I, or a salt thereof, wherein D is covalently attached to the remainder of Camptothecin-Linker compound via any suitable attachment site on D, optionally wherein a hydrogen atom of a hydroxyl, thiol, primary amine, or secondary amine of D is replaced with a bond to the remainder of the Camptothecin-Linker compound or a tertiary amine of D is quaternized to form a bond to the remainder of the Camptothecin-Linker compound.

47. The Camptothecin-Linker compound of claim 31, having the formula selected from the group consisting of formula (i), formula (ii); formula (iii), formula (iv), formula (v) and formula (vi), wherein A is a Connector Unit; and RL is a Glycoside (e.g., Glucuronide) Unit, optionally wherein the Glycoside Unit has the formula of: 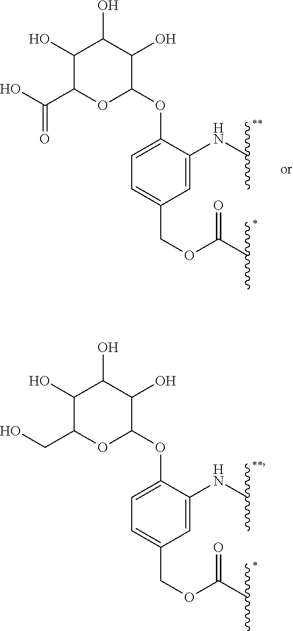 [SMILES: *Nc1cc(COC(*)=O)ccc1OC1OC(C(=O)O)C(O)C(O)C1O ; *Nc1cc(COC(*)=O)ccc1OC1OC(CO)C(O)C(O)C1O]
or optionally wherein the Glycoside Unit has the formula of: 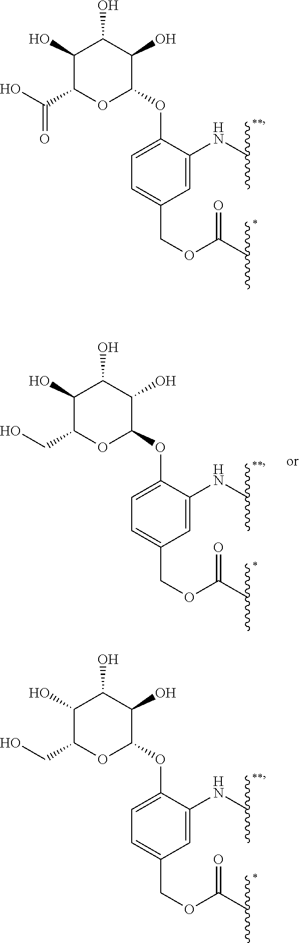 [SMILES: *Nc1cc(COC(*)=O)ccc1O[C@@H]1O[C@H](C(=O)O)[C@@H](O)[C@H](O)[C@H]1O ; *Nc1cc(COC(*)=O)ccc1O[C@H]1O[C@H](CO)[C@@H](O)[C@H](O)[C@@H]1O ; *Nc1cc(COC(*)=O)ccc1O[C@@H]1O[C@H](CO)[C@H](O)[C@H](O)[C@H]1O]
wherein the wavy line marked with a single asterisk (*) indicates the site of covalent attachment to D or to a Spacer Unit (Y); and the wavy line marked with a double asterisk (**) indicates the point of covalent attachment to A, B or S.

48-53. (canceled)

54. The Camptothecin-Linker Compound of claim 31 having formula (vii), formula (viii) or formula (ix), wherein A is a Connector Unit, or having formula (i), formula (iii), formula (x) or formula (xi), wherein A is a Connector Unit and RL is a Releasable linker other than a Glycoside (e.g., Glucuronide) Unit.

55. The Camptothecin-Linker Compound of claim 54 having formula (i), formula (iii) or formula (x), wherein
RL has the formula: 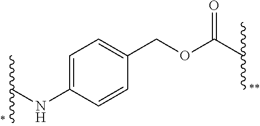 [SMILES: *Nc1ccc(COC(*)=O)cc1]
wherein
the wavy line marked with a double asterisk (**) indicates the site of covalent attachment to D; and
the wavy line marked with a single asterisk (*) indicates the point of covalent attachment to A, S* or W.

56. The Camptothecin-Linker Compound of claim 55 having formula (x) wherein W is an Amino Acid Unit selected from the group consisting of N-methyl-glycine (sarcosine), N-methyl-alanine, N-methyl-β-alanine, valine and N-methyl-valine.

57-60. (canceled)

61. A method of treating cancer in a subject in need thereof, comprising administering to the subject an effective amount of a Camptothecin Conjugate of claim 1, optionally said cancer is selected from the group consisting of lymphomas, leukemias, and solid tumors, optionally a lymphoma or a leukemia.

62. (canceled)

63. A pharmaceutically acceptable composition comprising a Camptothecin Conjugate of claim 1 and at least one pharmaceutically acceptable excipient.

64. (canceled)

65. A compound of Formula D_{0b} 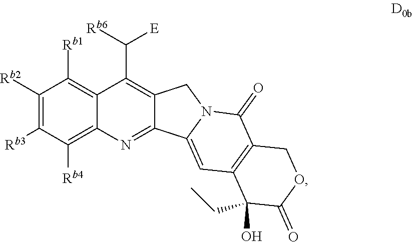
or a salt thereof, wherein
E is —OR^{b5} or —NR^{b5}R^{b5}′;
R^{b1} is selected from the group consisting of H, halogen, —CN, C₁-C₈ alkyl, C₁-C₈ haloalkyl, C₂-C₈ alkenyl, C₂-C₈ alkynyl, C₆-C₁₂ aryl, 5- to 12-membered heteroaryl, C₃-C₁₀ cycloalkyl, 3- to 10-membered heterocycloalkyl, (C₆-C₁₂ aryl)-C₂-C₈ alkenyl-, C₁-C₈ hydroxyalkyl, C₁-C₈ alkyl-C(O)—C₁-C₈ aminoalkyl-, C₁-C₈ aminoalkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}′, and —SR^{a}; each optionally substituted with C₁-C₃ alkyl, —OR^{a}, —NR^{a}R^{a}′, —C(O)R^{a}, and —SR^{a}; or
R^{b1} is combined with R^{b2}, R^{b5}, or R^{b6} and the intervening atoms to form a 5-, 6-, or 7-membered carbocyclo or heterocyclo;
R^{b2} is selected from the group consisting of H, halogen, C₁-C₈ alkyl, C₂-C₈ alkynyl, C₆-C₁₂ aryl, 5- to 12-membered heteroaryl, C₃-C₁₀ cycloalkyl, 3- to 10-membered heterocycloalkyl, C₁-C₈ haloalkyl, C₁-C₈ hydroxyalkyl, C₁-C₈ alkyl-S(O)₂—, C₁-C₈ aminoalkyl, C₁-C₈ alkyl-C(O)—C₁-C₈ aminoalkyl-, C₁-C₈ aminolkyl-C(O)—C₁-C₈ alkyl-, C₁-C₈ alkyl-NR^{a}—C(O)—, C₁-C₈ alkyl-C(O)—, C₁-C₈ alkyl-OC(O)—, C₁-C₈ alkyl-NR^{a}—C(O)—, C₁-C₈ alkyl-C(O)—NR^{a}—, C₁-C₈ alkyl-NR^{a}—C(O)O—, C₁-C₈ alkyl-OC(O)—NR^{a}—, C₆-C₁₂ aryl-C(O)—, C₆-C₁₂ aryl-O—C(O)—NR^{a}—, C₆-C₁₂ aryl-NR^{a}—C(O)—O—, —COOR^{a}, —OR^{a}, —NR^{a}R^{a}, and —SR^{a}; each optionally substituted with OR^{a}, —NR^{a}R^{a}, and —SR^{a}; or
R^{b2} is combined with R^{b1} or R^{b3} and the intervening atoms to form a 5- or 6-membered carbocyclo or heterocyclo; or
R^{b2} is combined with R^{b1} or R^{b3} and the intervening atoms to form 5- or 6-membered heterocyclo fused with 6-membered aryl;
R^{b3} is selected from the group consisting of H, halogen, C₁-C₆ alkyl, C₂-C₆ alkenyl, C₁-C₆ haloalkyl, —OR^{a}, —NR^{a}R^{a}, and —SR^{a};
R^{b4} is selected from the group consisting of H or halogen;
each R^{b5} and R^{b5}′ are independently selected from the group consisting of H, C₁-C₈ alkyl, C₁-C₈ hydroxyalkyl, C₁-C₈ alkyl-O-C₁-C₈ alkyl-, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀heterocycloalkyl)-C₁-C₄ alkyl-, C₁-C₆ hydroxyalkyl-heteroaryl-, phenyl, phenyl-C₁-C₄ alkyl-, diphenyl-C₁-C₄ alkyl-, heteroaryl, heteroaryl-C₁-C₄ alkyl-, C₁-C₆ alkoxy-C(O)—C₁-C₈ aminoalkyl-, C₁-C₆ alkoxy-C(O)-N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered ring having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
R^{b6} is H, or is taken together with R^{b1} and the intervening atoms to form a carbocyclo or heterocyclo; and
R^{a} and R^{a′}are each independently selected from the group consisting of H, C₁-C₆ alkyl, C₁-C₆ haloalkyl, C₁-C₆ alkyl-S(O)₂—, C₁-C₆ alkyl-C(O)—, C₁-C₆ aminoalkyl-C(O)—, and C₁-C₆ hydroxyalkyl-C(O)—,
wherein a) when R^{b2} is combined with R^{b3} and the intervening atoms to form a 1,3-dioxolane and E is —NR^{b5}R^{b5}′, then each R^{b5} and R^{b5}′ are independently selected from the group consisting of H, C₁-C₈ alkyl-O-C₁-C₈ alkyl-, C₁-C₈ alkyl-C(O) (C₃-C₁₀ cycloalkyl)-C₁-C₄ alkyl-, C₃-C₁₀ heterocycloalkyl, C₁-C₆ hydroxyalkyl-heteroaryl-, heteroaryl, heteroaryl-C₁-C₄ alkyl-, C₁-C₆alkoxy-C(O)—C₁-C₈ aminoalkyl-, C₁-C₆alkoxy-C(O)—N—(C₁-C₄ alkyl)amino-C₁-C₈ alkyl-, C₁-C₆alkoxy-C(O)—(C₃-C₁₀ heterocycloalkyl)-, C₁-C₆alkoxy-C(O)—(C₃-C₁₀ heterocycloalkyl)-C₁-C₈ alkyl-, C₁-C₄ alkyl-SO₂-C₁-C₈ alkyl-, NH₂—SO₂-C₁-C₈ alkyl-, (C₃-C₁₀ heterocycloalkyl)-C₁-C₄ hydroxyalkyl-, C₁-C₆alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ hetercycloalkyl-, or R^{b5} and R^{b5}′ are combined with the nitrogen atom to which they are attached to form a 5-, 6- or 7-membered heterocycle having 0 to 3 substituents independently selected from the group consisting of halogen, C₁-C₄ alkyl, —OH, —C₁-C₆ hydroxyalkyl, —OC₁-C₄ alkyl, —NH₂, —NH—C₁-C₄ alkyl, —N(C₁-C₄ alkyl)₂, C₁-C₆alkoxy-C(O)—NH—, C₁-C₆alkoxy-C(O)—C₁-C₈ aminoalkyl-, and C₁-C₈ aminoalkyl; or
R^{b5}′ is H and R^{b5} is combined with R^{b1} and the intervening atoms to form a 5- to 7-membered carbocyclo or heterocyclo; wherein the cycloalkyl, carbocyclo, heterocycloalkyl, heterocyclo, phenyl and heteroaryl portions of R^{b1}, R^{b2}, R^{b3}, R^{b4}, R^{b5} and R^{b5}′ are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂;
b) when R^{b2} is combined with R^{b3} and the intervening atoms to form a 1,3-dioxolane, E is not —OH; and
c) when R^{b2} is methyl and R^{b3} is F, then R^{b1} does not come together with R^{b6} and the intervening atoms to form a ring; and
d) D is not (S)-7-ethyl-7-hydroxy-14-((4-methylpiperazin-1-yl)methyl)-10,13-dihydro-11H-[1,3]dioxolo[4,5-g]pyrano[3′,4′:6,7]indolizino[1,2-b]quinoline-8,11(7H)-dione or (S)-7-ethyl-7-hydroxy-14-(morpholinomethyl)-10,13-dihydro-11H-[1,3]dioxolo[4,5-g]pyrano[3′,4′:6,7]indolizino[1,2-b]quinoline-8,11(7H)-dione, or a salt of any of the foregoing.

66-71. (canceled)

72. A compound selected from the compounds of Table I or a salt thereof.

73. The compound of claim 65, having Formula: 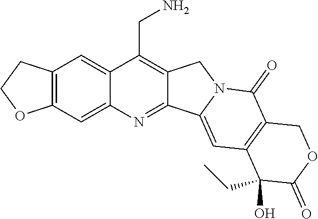 [SMILES: CC[C@@]1(O)C(=O)OCc2c1cc1n(c2=O)Cc2c-1nc1cc3c(cc1c2CN)CCO3]
or a salt thereof.

74-75. (canceled)

76. A Camptothecin-Linker compound of claim 31, wherein the compound is of Formula 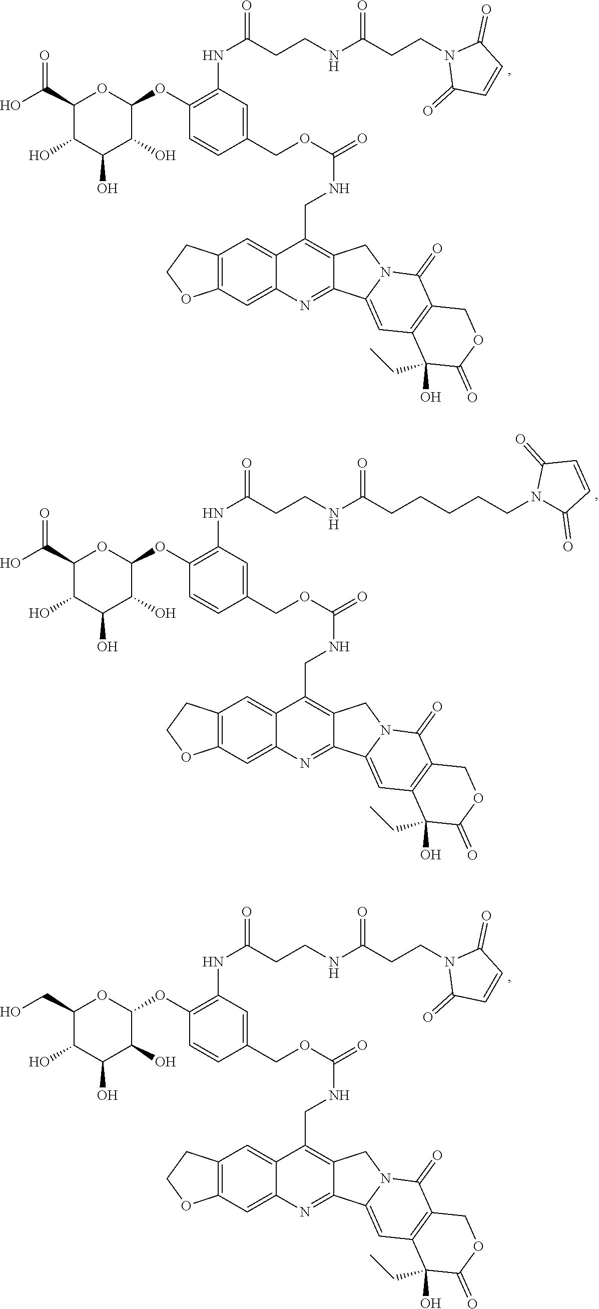 [SMILES: CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCO3 ; CC[C@@]1(O)C(=O)OCc2c1cc1n(c2=O)Cc2c-1nc1cc3c(cc1c2CNC(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1)CCO3 ; CC[C@@]1(O)C(=O)OCc2c1cc1n(c2=O)Cc2c-1nc1cc3c(cc1c2CNC(=O)OCc1ccc(O[C@H]2O[C@H](CO)[C@@H](O)[C@H](O)[C@@H]2O)c(NC(=O)CCNC(=O)CCN2C(=O)C=CC2=O)c1)CCO3] 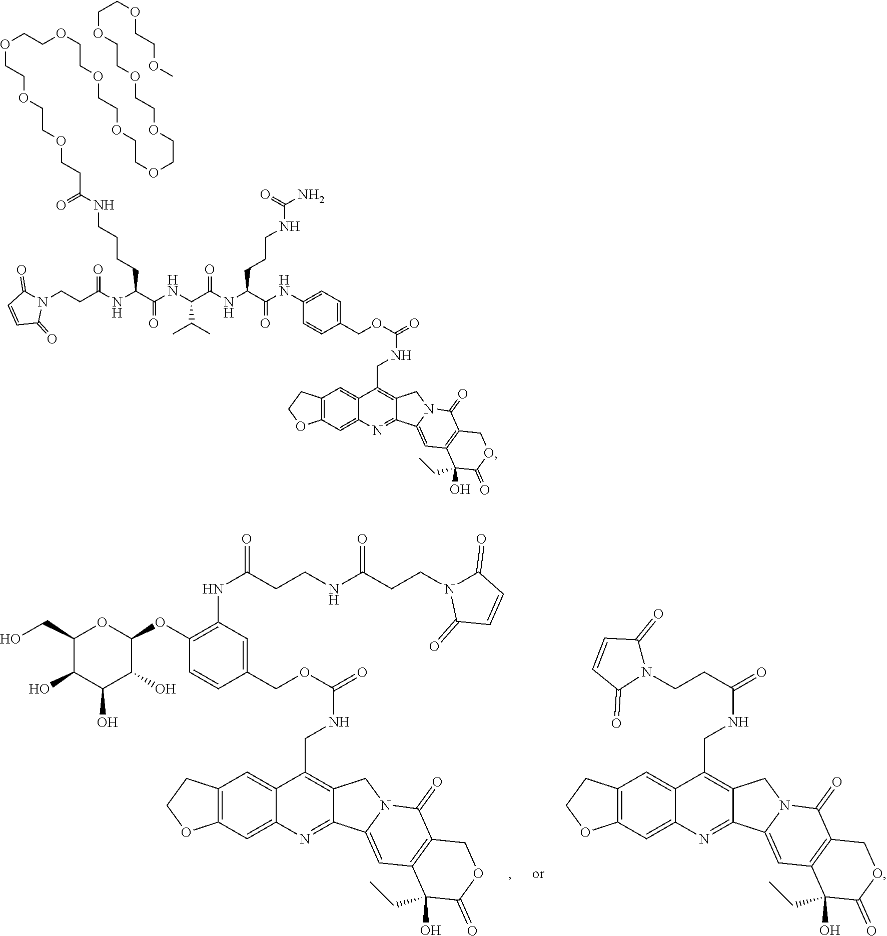 [SMILES: CC[C@@]1(O)C(=O)OCc2c1cc1n(c2=O)Cc2c-1nc1cc3c(cc1c2CNC(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOC)NC(=O)CCN2C(=O)C=CC2=O)C(C)C)cc1)CCO3 ; CC[C@@]1(O)C(=O)OCc2c1cc1n(c2=O)Cc2c-1nc1cc3c(cc1c2CNC(=O)OCc1ccc(O[C@@H]2O[C@H](CO)[C@H](O)[C@H](O)[C@H]2O)c(NC(=O)CCNC(=O)CCN2C(=O)C=CC2=O)c1)CCO3 ; CC[C@@]1(O)C(=O)OCc2c1cc1n(c2=O)Cc2c-1nc1cc3c(cc1c2CNC(=O)CCN1C(=O)C=CC1=O)CCO3]
or a salt thereof.

77. The compound of claim 31, having Formula 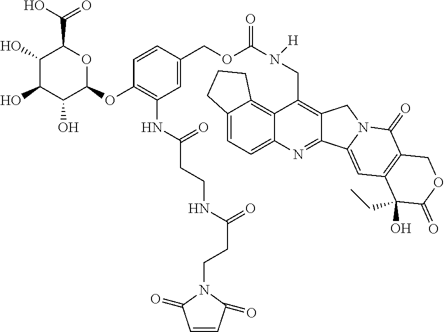 [SMILES: CC[C@@]1(O)C(=O)OCc2c1cc1n(c2=O)Cc2c-1nc1ccc3c(c1c2CNC(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)CCC3]
or a salt thereof.

78. The compound of claim 31, having Formula 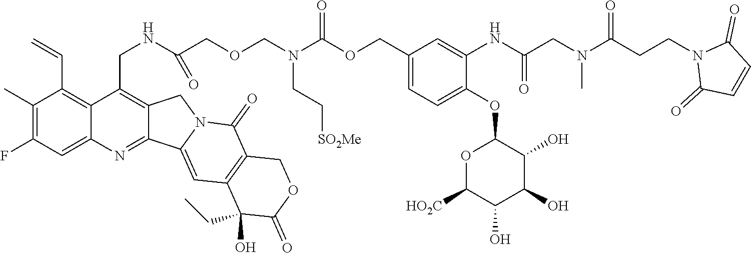 [SMILES: C=Cc1c(C)c(F)cc2nc3c(c(CNC(=O)COCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)c12)Cn1c-3cc2c(c1=O)COC(=O)[C@]2(O)CC]
or a salt thereof.

79. The compound of claim 1, having Formula 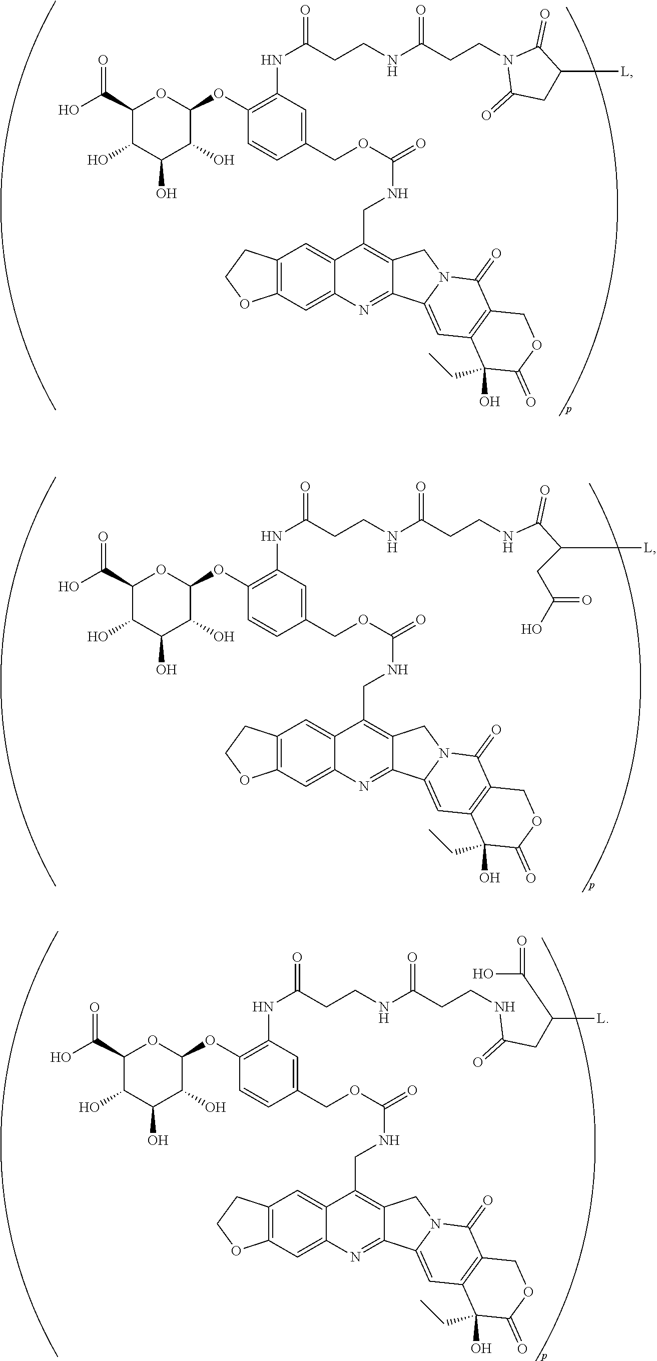 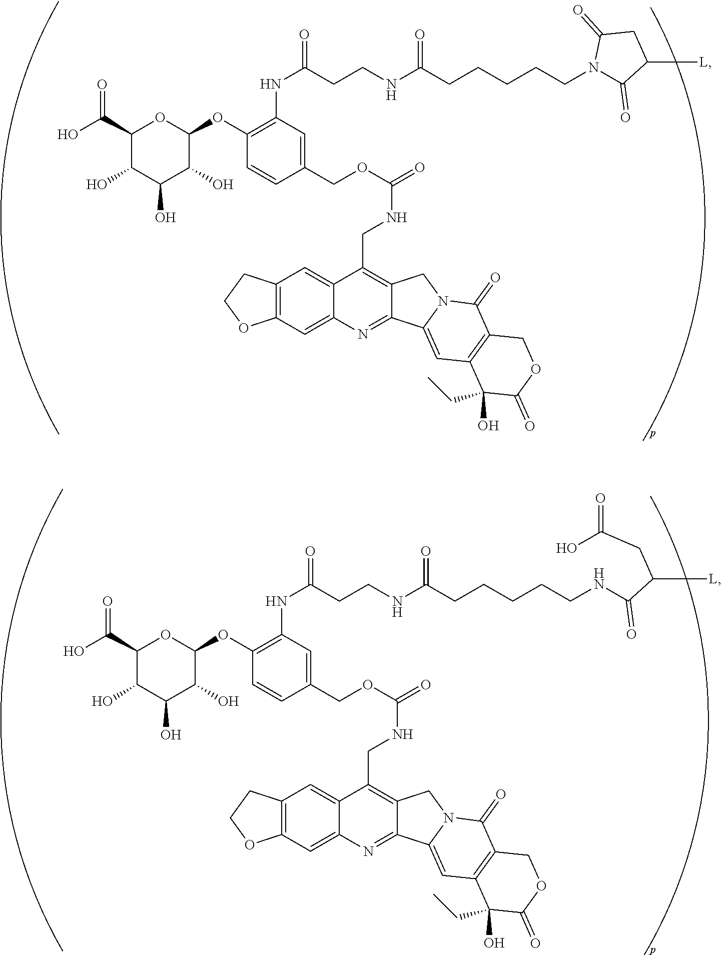 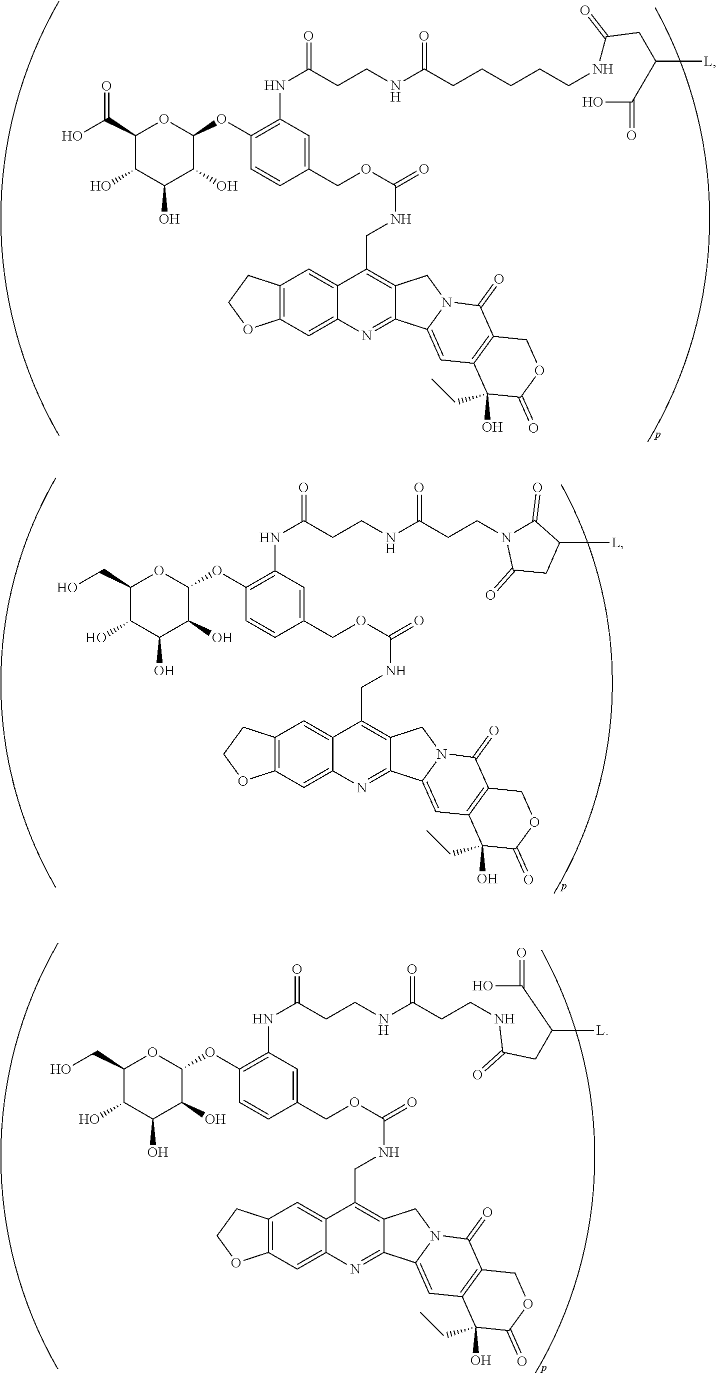 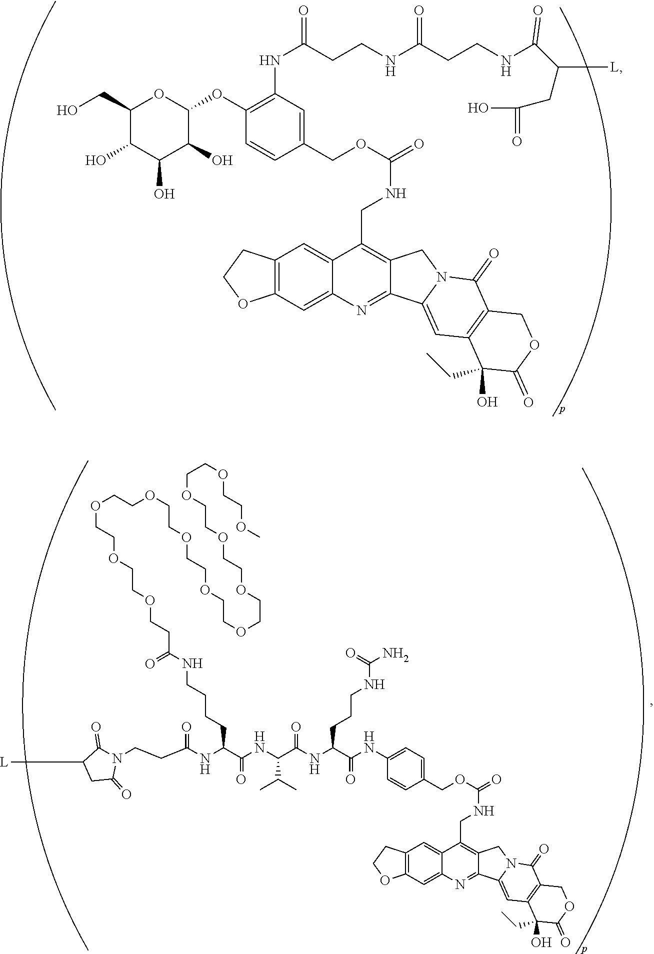 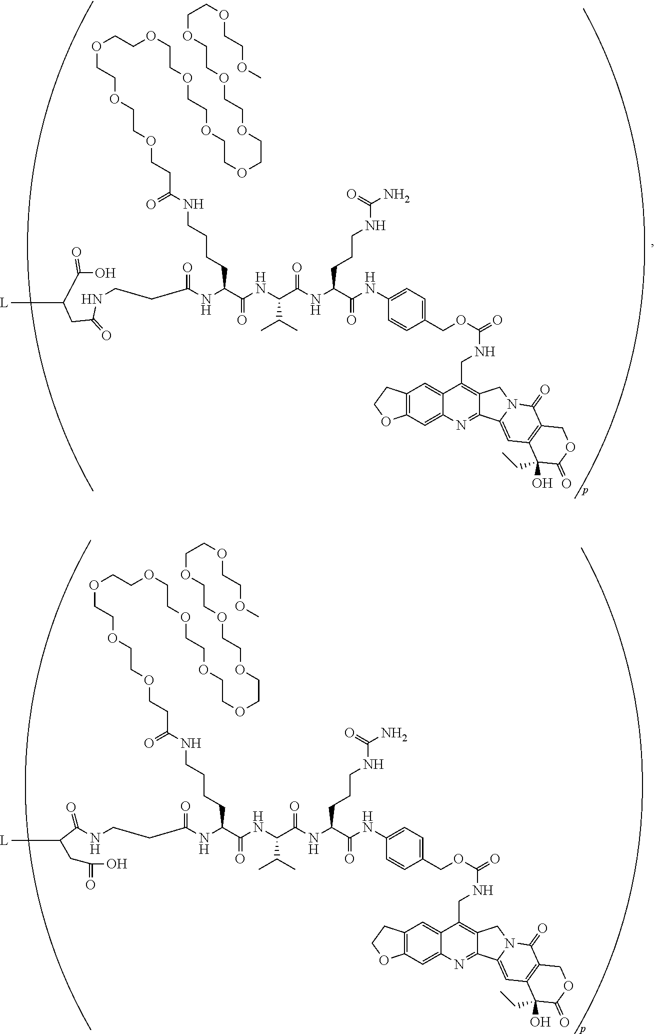 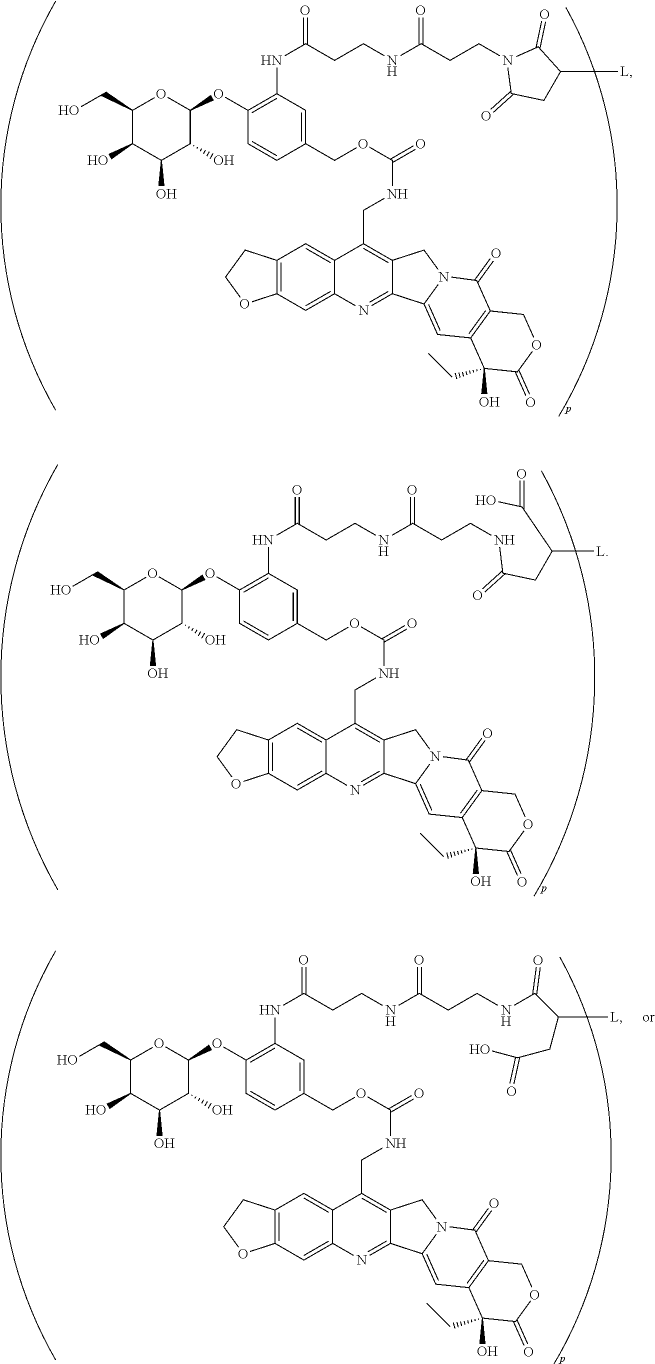 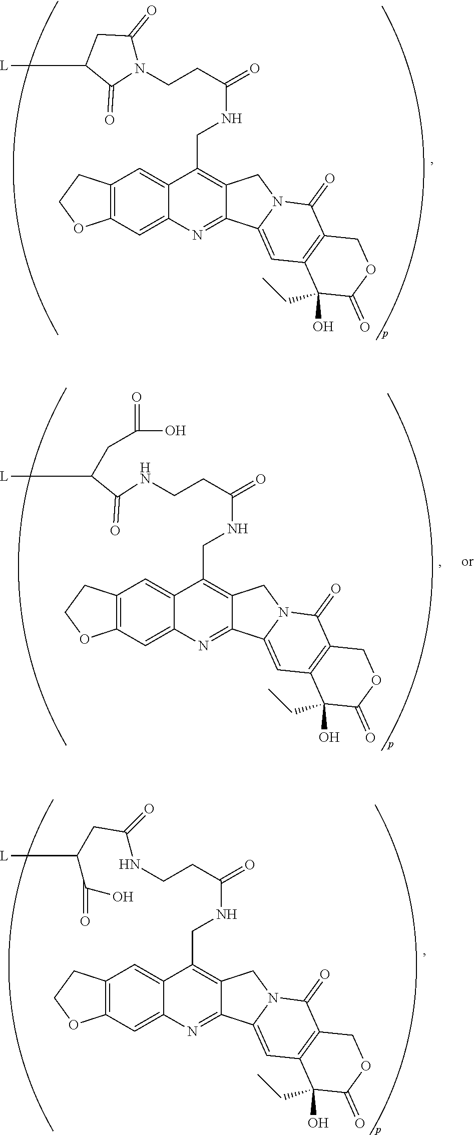
or a salt thereof.

80. The compound of claim 1, having Formula 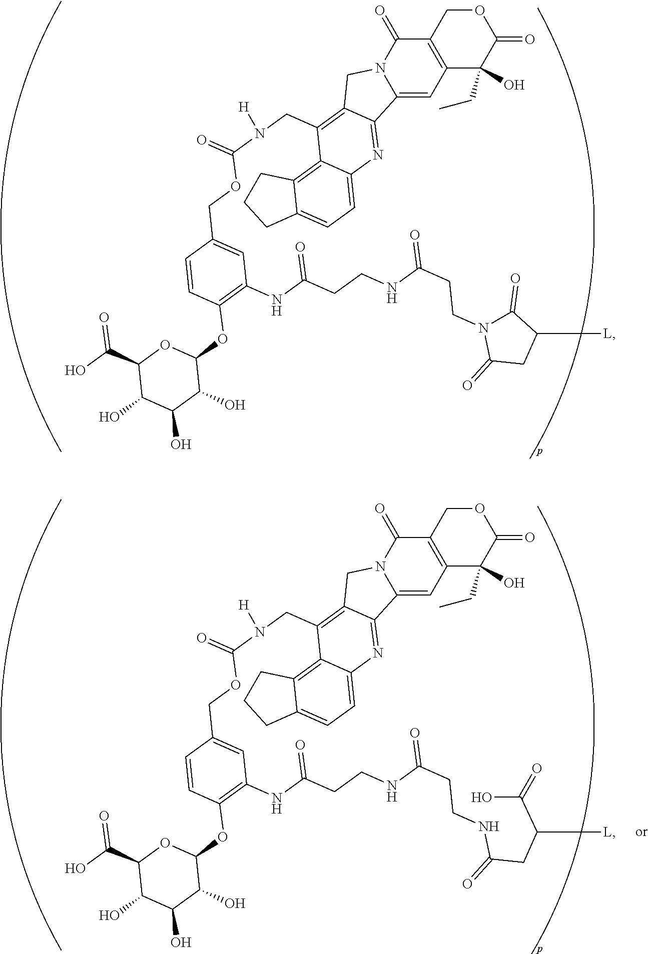 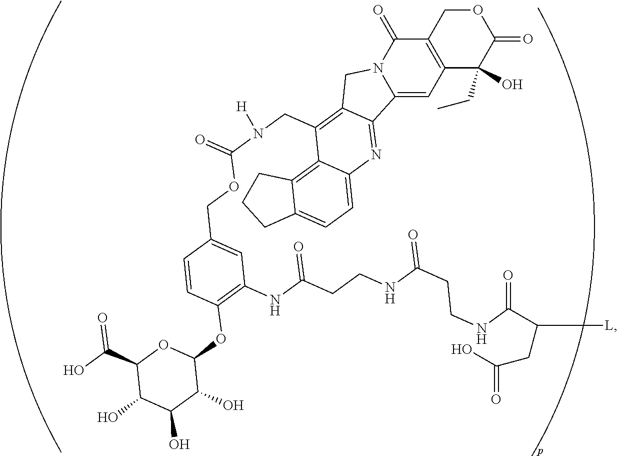
or a salt thereof.

81. The compound of claim 1, having Formula 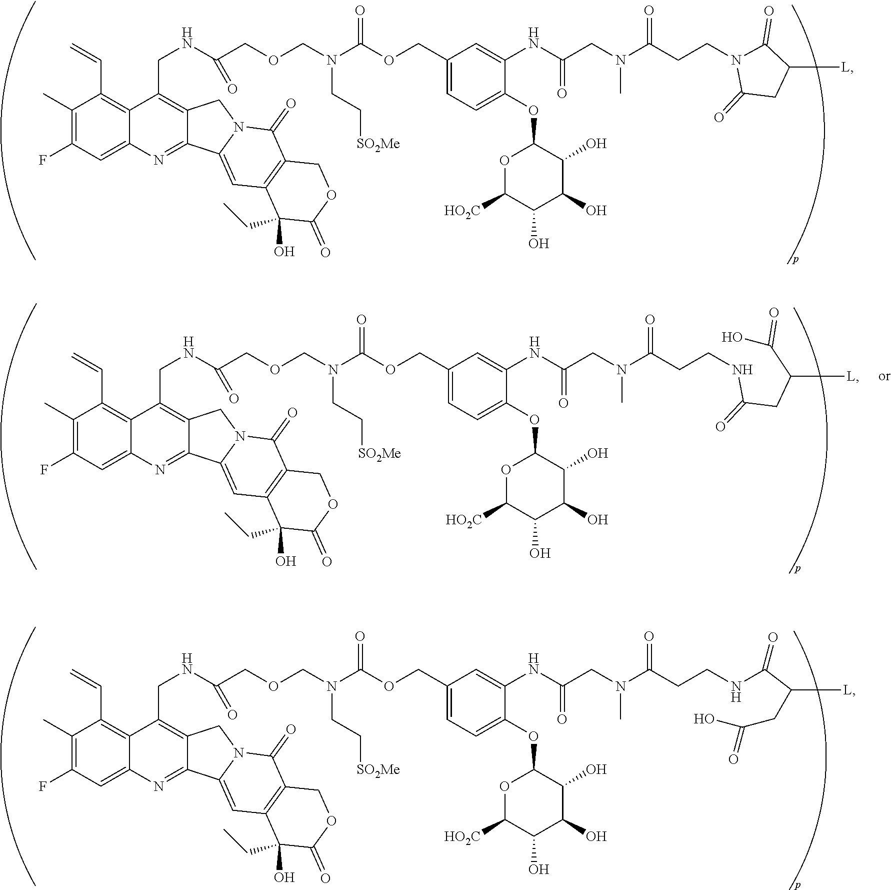
or a salt thereof.

82. The Camptothecin-Linker compound of claim 31, wherein the compound is a compound of Table II or a salt thereof.

83. The Camptothecin Conjugate of claim 1, wherein the Conjugate comprises a Ligand attached to a succinimide moiety or a succinic acid-amide moiety of a Camptothecin-Linker moiety, wherein the Camptothecin-Linker moiety comprises a compound of Table II, wherein a maleimide moiety of the Camptothecin-Linker moiety is replaced by the succinimide or succinic acid-amide moiety.

84. The compound of claim 65, wherein the compound has the formula of D₀-I″ 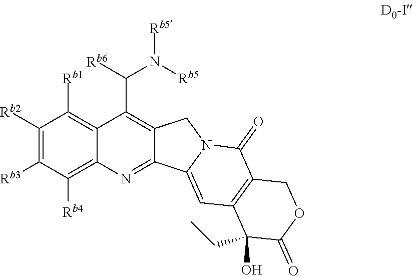
or a salt thereof,
wherein when R^{b2} is combined with R^{b3} and the intervening atoms to form a 5-, 6-, or 7-membered heterocyclo, the heterocyclo has no more than one O.

85. (canceled)


SEQUENCE LISTING (1226 sequences)

SEQ ID NO: 1 - protein, 5 aa, synthetic construct
DYYIT

SEQ ID NO: 2 - protein, 17 aa, synthetic construct
WIYPGSGNTKYNEKFKG

SEQ ID NO: 3 - protein, 8 aa, synthetic construct
YGNYWFAY

SEQ ID NO: 4 - protein, 15 aa, synthetic construct
KASQSVDFDGDSYMN

SEQ ID NO: 5 - protein, 7 aa, synthetic construct
AASNLES

SEQ ID NO: 6 - protein, 9 aa, synthetic construct
QQSNEDPWT

SEQ ID NO: 7 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 13 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 14 - protein, 448 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NYGMN

SEQ ID NO: 17 - protein, 17 aa, synthetic construct
WINTYTGEPTYTDDFKG

SEQ ID NO: 18 - protein, 12 aa, synthetic construct
GGFGSSYWYFDV

SEQ ID NO: 19 - protein, 11 aa, synthetic construct
KASQDVSIAVA

SEQ ID NO: 20 - protein, 7 aa, synthetic construct
SASYRYT

SEQ ID NO: 21 - protein, 9 aa, synthetic construct
QQHYITPLT

SEQ ID NO: 22 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 23 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 24 - protein, 5 aa, synthetic construct
TAGMQ

SEQ ID NO: 25 - protein, 17 aa, synthetic construct
WINTHSGVPKYAEDFKG

SEQ ID NO: 26 - protein, 12 aa, synthetic construct
SGFGSSYWYFDV

SEQ ID NO: 27 - protein, 11 aa, synthetic construct
KASQDVSTAVA

SEQ ID NO: 28 - protein, 7 aa, synthetic construct
SASYRYT

SEQ ID NO: 29 - protein, 9 aa, synthetic construct
QQHYITPLT

SEQ ID NO: 30 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 31 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 32 - protein, 5 aa, synthetic construct
SQNIY

SEQ ID NO: 33 - protein, 17 aa, synthetic construct
YIEPYNVVPMYNPKFKG

SEQ ID NO: 34 - protein, 8 aa, synthetic construct
SGSSNFDY

SEQ ID NO: 35 - protein, 12 aa, synthetic construct
SASSSISSHYLH

SEQ ID NO: 36 - protein, 7 aa, synthetic construct
RTSNLAS

SEQ ID NO: 37 - protein, 9 aa, synthetic construct
QQGSSLPLT

SEQ ID NO: 38 - protein, 117 aa, synthetic construct
EIQLVQSGAEVKKPGASVKVSCKASGYAFTSQNIYWVRQAPGQGLEWIGYIEPYNVVPMYNPKFKGRATLTVDKSTSTAYLELSSLRSEDTAVYYCARSGSSNFDYWGQGTLVTVSS

SEQ ID NO: 39 - protein, 108 aa, synthetic construct
DIQLTQSPSSLSASVGDRVTITCSASSSISSHYLHWYQQKPGKSPKLLIYRTSNLASGVPSRFSGSGSGTDYTLTISSLQPEDFATYYCQQGSSLPLTFGQGTKVEIK

SEQ ID NO: 40 - protein, 5 aa, synthetic construct
NYAMH

SEQ ID NO: 41 - protein, 17 aa, synthetic construct
LIWYDGSNKFYGDSVKG

SEQ ID NO: 42 - protein, 6 aa, synthetic construct
EGSGHY

SEQ ID NO: 43 - protein, 11 aa, synthetic construct
RASQGISSALA

SEQ ID NO: 44 - protein, 7 aa, synthetic construct
DASSLES

SEQ ID NO: 45 - protein, 9 aa, synthetic construct
QQFNSYPIT

SEQ ID NO: 46 - protein, 115 aa, synthetic construct
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSS

SEQ ID NO: 47 - protein, 107 aa, synthetic construct
AIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIK

SEQ ID NO: 48 - protein, 5 aa, synthetic construct
NYAMS

SEQ ID NO: 49 - protein, 17 aa, synthetic construct
YISPGGDYIYYADSVKG

SEQ ID NO: 50 - protein, 12 aa, synthetic construct
DRRHYGSYAMDY

SEQ ID NO: 51 - protein, 16 aa, synthetic construct
RSSKSLLHSNLNTYLY

SEQ ID NO: 52 - protein, 7 aa, synthetic construct
RMSNLAS

SEQ ID NO: 53 - protein, 9 aa, synthetic construct
MQHLEYPFT

SEQ ID NO: 54 - protein, 121 aa, synthetic construct
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 55 - protein, 112 aa, synthetic construct
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIK

SEQ ID NO: 56 - protein, 5 aa, synthetic construct
TYAFH

SEQ ID NO: 57 - protein, 17 aa, synthetic construct
GIVPIFGTLKYAQKFQD

SEQ ID NO: 58 - protein, 11 aa, synthetic construct
AIQLEGRPFDH

SEQ ID NO: 59 - protein, 11 aa, synthetic construct
RASQGITSYLA

SEQ ID NO: 60 - protein, 7 aa, synthetic construct
AASALQS

SEQ ID NO: 61 - protein, 10 aa, synthetic construct
QQVNRGAAIT

SEQ ID NO: 62 - protein, 120 aa, synthetic construct
QVQLVQSGAEVKKPGSSVRVSCRASGGSSTTYAFHWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 63 - protein, 108 aa, synthetic construct
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIK

SEQ ID NO: 64 - protein, 5 aa, synthetic construct
TYAFH

SEQ ID NO: 65 - protein, 17 aa, synthetic construct
GIVPIFGTLKYAQKFQD

SEQ ID NO: 66 - protein, 11 aa, synthetic construct
AIQLEGRPFDH

SEQ ID NO: 67 - protein, 11 aa, synthetic construct
RASQGITSYLA

SEQ ID NO: 68 - protein, 7 aa, synthetic construct
AASALQS

SEQ ID NO: 69 - protein, 10 aa, synthetic construct
QQVNRGAAIT

SEQ ID NO: 70 - protein, 120 aa, synthetic construct; identical to SEQ ID NO: 62

SEQ ID NO: 71 - protein, 108 aa, synthetic construct; identical to SEQ ID NO: 63

SEQ ID NO: 72 - protein, 5 aa, synthetic construct
RYTMH

SEQ ID NO: 73 - protein, 17 aa, synthetic construct
VISFDGSNKYYVDSVKG

SEQ ID NO: 74 - protein, 10 aa, synthetic construct
EARGSYAFDI

SEQ ID NO: 75 - protein, 11 aa, synthetic construct
RASQSVSSYLA

SEQ ID NO: 76 - protein, 7 aa, synthetic construct
DASNRAT

SEQ ID NO: 77 - protein, 10 aa, synthetic construct
QQRSNWPPFT

SEQ ID NO: 78 - protein, 119 aa, synthetic construct
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 79 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 80 - protein, 5 aa, synthetic construct
SFWMH

SEQ ID NO: 81 - protein, 17 aa, synthetic construct
YINPRSGYTEYNEIFRD

SEQ ID NO: 82 - protein, 9 aa, synthetic construct
FLGRGAMDY

SEQ ID NO: 83 - protein, 11 aa, synthetic construct
RASQDISNYLA

SEQ ID NO: 84 - protein, 7 aa, synthetic construct
YTSKIHS

SEQ ID NO: 85 - protein, 9 aa, synthetic construct
QQGNTFPYT

SEQ ID NO: 86 - protein, 118 aa, synthetic construct [sequence omitted]

SEQ ID NO: 87 - protein, 107 aa, synthetic construct [sequence omitted]

SEQ ID NO: 88 - protein, 7 aa, synthetic construct
TSSYYWG

SEQ ID NO: 89 - protein, 16 aa, synthetic construct
TIYYNGSTYYSPSLKS

SEQ ID NO: 90 - protein, 11 aa, synthetic construct
QGYDIKINIDV

SEQ ID NO: 91 - protein, 11 aa, synthetic construct
RASQSVSSYLA

SEQ ID NO: 92 - protein, 7 aa, synthetic construct
VASNRAT

SEQ ID NO: 93 - protein, 9 aa, synthetic construct
QQRSNWPLT

SEQ ID NO: 94 - protein, 121 aa, synthetic construct
QLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSS

SEQ ID NO: 95 - protein, 107 aa, synthetic construct
EIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIK

SEQ ID NO: 96 - protein, 5 aa, synthetic construct
SSWMN

SEQ ID NO: 97 - protein, 17 aa, synthetic construct
RIYPGDGNTHYAQKFQG

SEQ ID NO: 98 - protein, 8 aa, synthetic construct
GYLDPMDY

SEQ ID NO: 99 - protein, 11 aa, synthetic construct
QASQGINNYLN

SEQ ID NO: 100 - protein, 7 aa, synthetic construct
YTSGLHA

SEQ ID NO: 101 - protein, 9 aa, synthetic construct
QQYSILPWT

SEQ ID NO: 102 - protein, 117 aa, synthetic construct
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 103 - protein, 107 aa, synthetic construct
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 104 - protein, 5 aa, synthetic construct
SYGMH

SEQ ID NO: 105 - protein, 17 aa, synthetic construct
VISYDGSNKYYADSVKG

SEQ ID NO: 106 - skipped

SEQ ID NO: 107 - protein, 11 aa, synthetic construct
RTSQSISSYLN

SEQ ID NO: 108 - protein, 7 aa, synthetic construct
WASTRES

SEQ ID NO: 109 - protein, 9 aa, synthetic construct
QQSYDIPYT

SEQ ID NO: 110 - protein, 127 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 111 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 112 - protein, 5 aa, synthetic construct
NYWMS

SEQ ID NO: 113 - protein, 17 aa, synthetic construct
NIKQDGSEKFYADSVKG

SEQ ID NO: 114 - protein, 9 aa, synthetic construct
VGPSWEQDY

SEQ ID NO: 115 - protein, 14 aa, synthetic construct
TGSSSNIGSYYGVH

SEQ ID NO: 116 - protein, 7 aa, synthetic construct
SDTNRPS

SEQ ID NO: 117 - protein, 11 aa, synthetic construct
QSYDKGFGHRV

SEQ ID NO: 118 - protein, 118 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 119 - protein, 94 aa, synthetic construct
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYD

SEQ ID NO: 120 - protein, 5 aa, synthetic construct
SYAIS

SEQ ID NO: 121 - protein, 17 aa, synthetic construct
GIIPIFGTANYAQKFQG

SEQ ID NO: 122 - protein, 6 aa, synthetic construct
GLLWNY

SEQ ID NO: 123 - protein, 11 aa, synthetic construct
RASQSVSSNLA

SEQ ID NO: 124 - protein, 7 aa, synthetic construct
GASTTAS

SEQ ID NO: 125 - protein, 11 aa, synthetic construct
QQYNNWPPAYT

SEQ ID NO: 126 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 127 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 128 - protein, 5 aa, synthetic construct
NYGMN

SEQ ID NO: 129 - protein, 17 aa, synthetic construct
WINTYTGEPTYGEDFKG

SEQ ID NO: 130 - protein, 7 aa, synthetic construct
FGNYVDY

SEQ ID NO: 131 - protein, 16 aa, synthetic construct
RSSKNLLHSNGITYLY

SEQ ID NO: 132 - protein, 7 aa, synthetic construct
QMSNLAS

SEQ ID NO: 133 - protein, 9 aa, synthetic construct
AQNLEIPRT

SEQ ID NO: 134 - protein, 116 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 135 - protein, 112 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 136 - protein, 5 aa, synthetic construct
KYGMN

SEQ ID NO: 137 - protein, 17 aa, synthetic construct
WINTYTEEPTYGDDFKG

SEQ ID NO: 138 - protein, 7 aa, synthetic construct
FGSAVDY

SEQ ID NO: 139 - protein, 16 aa, synthetic construct
RSSKSLLHSNGITYLY

SEQ ID NO: 140 - protein, 7 aa, synthetic construct
QMSNRAS

SEQ ID NO: 141 - protein, 9 aa, synthetic construct
AQNLELPRT

SEQ ID NO: 142 - protein, 116 aa, synthetic construct
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 143 - protein, 113 aa, synthetic construct
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKR

SEQ ID NO: 144 - protein, 5 aa, synthetic construct
DYSMH

SEQ ID NO: 145 - protein, 17 aa, synthetic construct
WINTETGEPTYADDFKG

SEQ ID NO: 146 - protein, 4 aa, synthetic construct
TAVY

SEQ ID NO: 147 - protein, 11 aa, synthetic construct
RASQEISVSLS

SEQ ID NO: 148 - protein, 7 aa, synthetic construct
ATSTLDS

SEQ ID NO: 149 - protein, 9 aa, synthetic construct
LQYASYPWT

SEQ ID NO: 150 - protein, 113 aa, synthetic construct
QVKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 151 - protein, 108 aa, synthetic construct
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKR

SEQ ID NO: 152 - protein, 5 aa, synthetic construct
NYGMN

SEQ ID NO: 153 - protein, 17 aa, synthetic construct
WINTYSGEPRYADDFKG

SEQ ID NO: 154 - protein, 9 aa, synthetic construct
DYGRWYFDV

SEQ ID NO: 155 - protein, 10 aa, synthetic construct
RASSSVSHMH

SEQ ID NO: 156 - protein, 7 aa, synthetic construct
ATSNLAS

SEQ ID NO: 157 - protein, 9 aa, synthetic construct
QQWSSTPRT

SEQ ID NO: 158 - protein, 118 aa, synthetic construct
QIQLVQSGSELKKPGASVKVSCKASGYTFTNYGMNWVRQAPGQDLKWMGWINTYSGEPRYADDFKGRFVFSLDKSVNTAYLQISSLKAEDTAVYYCARDYGRWYFDVWGQGTTVTVSS

SEQ ID NO: 159 - protein, 107 aa, synthetic construct
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIKR

SEQ ID NO: 160 - protein, 5 aa, synthetic construct
RYWMS

SEQ ID NO: 161 - protein, 17 aa, synthetic construct
EINPDSSTINYAPSLKD

SEQ ID NO: 162 - protein, 10 aa, synthetic construct
PDGNYWYFDV

SEQ ID NO: 163 - protein, 11 aa, synthetic construct
KASQDVGIAVA

SEQ ID NO: 164 - protein, 7 aa, synthetic construct
WASTRHT

SEQ ID NO: 165 - protein, 9 aa, synthetic construct
QQYSSYPYT

SEQ ID NO: 166 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 167 - protein, 108 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 168 - protein, 5 aa, synthetic construct
SFAMS

SEQ ID NO: 169 - protein, 17 aa, synthetic construct
AISGSGGGTYYADSVKG

SEQ ID NO: 170 - protein, 13 aa, synthetic construct
DKILWFGEPVFDY

SEQ ID NO: 171 - protein, 11 aa, synthetic construct
RASQSVSSYLA

SEQ ID NO: 172 - protein, 7 aa, synthetic construct
DASNRAT

SEQ ID NO: 173 - protein, 9 aa, synthetic construct
QQRSNWPPT

SEQ ID NO: 174 - protein, 122 aa, synthetic construct [sequence omitted]

SEQ ID NO: 175 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 176 - protein, 5 aa, synthetic construct
SYRMH

SEQ ID NO: 177 - protein, 17 aa, synthetic construct
YINPSTGYTEYNQKFKD

SEQ ID NO: 178 - protein, 7 aa, synthetic construct
GGGVFDY

SEQ ID NO: 179 - protein, 10 aa, synthetic construct
SASSSISYMH

SEQ ID NO: 180 - protein, 7 aa, synthetic construct
TTSNLAS

SEQ ID NO: 181 - protein, 9 aa, synthetic construct
HQRSTYPLT

SEQ ID NO: 182 - protein, 116 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 183 - protein, 106 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 184 - protein, 4 aa, synthetic construct
SYWM

SEQ ID NO: 185 - protein, 17 aa, synthetic construct
EIIPINGHTNYNEKFKS

SEQ ID NO: 186 - protein, 14 aa, synthetic construct
GGYYYYGSRDYFDY

SEQ ID NO: 187 - protein, 15 aa, synthetic construct
KASQSVDYDGDSYMN

SEQ ID NO: 188 - protein, 7 aa, synthetic construct
AASDLES

SEQ ID NO: 189 - protein, 9 aa, synthetic construct
QQSHEDPFT

SEQ ID NO: 190 - protein, 123 aa, synthetic construct
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 191 - protein, 111 aa, synthetic construct
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 192 - protein, 4 aa, synthetic construct
SYWM

SEQ ID NO: 193 - protein, 17 aa, synthetic construct
EIIPIFGHTNYNEKFKS

SEQ ID NO: 194 - protein, 14 aa, synthetic construct
GGYYYYPRQGFLDY

SEQ ID NO: 195 - protein, 16 aa, synthetic construct
KASQSVDYDSGDSYMN

SEQ ID NO: 196 - protein, 7 aa, synthetic construct
AASDLES

SEQ ID NO: 197 - protein, 9 aa, synthetic construct
QQSHEDPFT

SEQ ID NO: 198 - protein, 123 aa, synthetic construct
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 199 - protein, 111 aa, synthetic construct
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 200 - protein, 4 aa, synthetic construct
YGMN

SEQ ID NO: 201 - protein, 16 aa, synthetic construct
YISSSSSTIYADSVKG

SEQ ID NO: 202 - protein, 6 aa, synthetic construct
GPGMDV

SEQ ID NO: 203 - protein, 17 aa, synthetic construct
KSSQSVLYSSNNKNYLA

SEQ ID NO: 204 - protein, 7 aa, synthetic construct
WASTRES

SEQ ID NO: 205 - protein, 10 aa, synthetic construct
QQYYSTPQLT

SEQ ID NO: 206 - protein, 112 aa, synthetic construct
QVQLQQSGGGVVQPGRSLGLSCAASFTFSSYGMNWVRQAPGKGLEWVSYISSSSSTIYADSVKGRFTISRDNSKNTLYLQMNSLRAEDTAVYYCARGPGMDVWGQGTTVTVS

SEQ ID NO: 207 - protein, 114 aa, synthetic construct
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIK

SEQ ID NO: 208 - protein, 7 aa, synthetic construct
TSGMGVG

SEQ ID NO: 209 - protein, 16 aa, synthetic construct
HIWWDDDKRYNPALKS

SEQ ID NO: 210 - protein, 10 aa, synthetic construct
MELWSYYFDY

SEQ ID NO: 211 - protein, 10 aa, synthetic construct
SASSSVSYMH

SEQ ID NO: 212 - protein, 7 aa, synthetic construct
DTSKLAS

SEQ ID NO: 213 - protein, 9 aa, synthetic construct
FQGSVYPFT

SEQ ID NO: 214 - protein, 120 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 215 - protein, 107 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 216 - protein, 5 aa, synthetic construct
NYGMN

SEQ ID NO: 217 - protein, 17 aa, synthetic construct
WINTYTGEPTYADAFKG

SEQ ID NO: 218 - protein, 9 aa, synthetic construct
DYGDYGMDY

SEQ ID NO: 219 - protein, 15 aa, synthetic construct
RASKSVSTSGYSFMH

SEQ ID NO: 220 - protein, 7 aa, synthetic construct
LASNLES

SEQ ID NO: 221 - protein, 9 aa, synthetic construct
QHSREVPWT

SEQ ID NO: 222 - protein, 118 aa, synthetic construct; identical to SEQ ID NO: 12

SEQ ID NO: 223 - protein, 111 aa, synthetic construct; identical to SEQ ID NO: 13

SEQ ID NO: 224 - protein, 6 aa, synthetic construct
SGYSWH

SEQ ID NO: 225 - protein, 16 aa, synthetic construct
YIHSSGSTNYNPSLKS

SEQ ID NO: 226 - protein, 7 aa, synthetic construct
YDDYFEY

SEQ ID NO: 227 - protein, 11 aa, synthetic construct
KASQNVGFNVA

SEQ ID NO: 228 - protein, 7 aa, synthetic construct
SASYRYS

SEQ ID NO: 229 - protein, 9 aa, synthetic construct
QQYNWYPFT

SEQ ID NO: 230 - protein, 116 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 231 - protein, 107 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 232 - protein, 5 aa, synthetic construct
NYWIE

SEQ ID NO: 233 - protein, 17 aa, synthetic construct
EILPGTGRTIYNEKFKG

SEQ ID NO: 234 - protein, 13 aa, synthetic construct
RDYYGNFYYAMDY

SEQ ID NO: 235 - protein, 11 aa, synthetic construct
SASQGINNYLN

SEQ ID NO: 236 - protein, 7 aa, synthetic construct
YTSTLQS

SEQ ID NO: 237 - protein, 9 aa, synthetic construct
QQYSKLPRT

SEQ ID NO: 238 - protein, 122 aa, synthetic construct
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 239 - protein, 107 aa, synthetic construct
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 240 - protein, 7 aa, synthetic construct
TYGMGVG

SEQ ID NO: 241 - protein, 16 aa, synthetic construct
NIWWSEDKHYSPSLKS

SEQ ID NO: 242 - protein, 11 aa, synthetic construct
IDYGNDYAFTY

SEQ ID NO: 243 - protein, 16 aa, synthetic construct
RSSKSLLHSNGITYLY

SEQ ID NO: 244 - protein, 7 aa, synthetic construct
QMSNLAS

SEQ ID NO: 245 - protein, 9 aa, synthetic construct
AQNLELPYT

SEQ ID NO: 246 - protein, 121 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 247 - protein, 112 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 248 - protein, 5 aa, synthetic construct
RYTMH

SEQ ID NO: 249 - protein, 17 aa, synthetic construct
VISFDGSNKYYVDSVKG

SEQ ID NO: 250 - protein, 10 aa, synthetic construct
EARGSYAFDI

SEQ ID NO: 251 - protein, 11 aa, synthetic construct
RASQSVSSYLA

SEQ ID NO: 252 - protein, 7 aa, synthetic construct
DASNRAT

SEQ ID NO: 253 - protein, 10 aa, synthetic construct
QQRSNWPPFT

SEQ ID NO: 254 - protein, 119 aa, synthetic construct; identical to SEQ ID NO: 78

SEQ ID NO: 255 - protein, 108 aa, synthetic construct; identical to SEQ ID NO: 79

SEQ ID NO: 256 - protein, 5 aa, synthetic construct
SFGMH

SEQ ID NO: 257 - protein, 17 aa, synthetic construct
YISSGSFTIYYADSVKG

SEQ ID NO: 258 - protein, 9 aa, synthetic construct
MRKGYAMDY

SEQ ID NO: 259 - protein, 16 aa, synthetic construct
RSSQIIIHSDGNTYLE

SEQ ID NO: 260 - protein, 7 aa, synthetic construct
KVSNRFS

SEQ ID NO: 261 - protein, 9 aa, synthetic construct
FQGSHVPHT

SEQ ID NO: 262 - protein, 118 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 263 - protein, 112 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 264 - protein, 5 aa, synthetic construct
NYGVN

SEQ ID NO: 265 - protein, 17 aa, synthetic construct
WINPNTGEPTFDDDFKG

SEQ ID NO: 266 - protein, 11 aa, synthetic construct
SRGKNEAWFAY

SEQ ID NO: 267 - protein, 16 aa, synthetic construct
RSSQSLVHRNGNTYLH

SEQ ID NO: 268 - protein, 7 aa, synthetic construct
TVSNRFS

SEQ ID NO: 269 - protein, 9 aa, synthetic construct
SQSSHVPPT

SEQ ID NO: 270 - protein, 120 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 271 - protein, 112 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 272 - protein, 5 aa, synthetic construct
TYWMS

SEQ ID NO: 273 - protein, 17 aa, synthetic construct
EIHPDSSTINYAPSLKD

SEQ ID NO: 274 - protein, 10 aa, synthetic construct
LYFGFPWFAY

SEQ ID NO: 275 - protein, 11 aa, synthetic construct
KASQDVGTSVA

SEQ ID NO: 276 - protein, 7 aa, synthetic construct
WTSTRHT

SEQ ID NO: 277 - protein, 8 aa, synthetic construct
QQYSLYRS

SEQ ID NO: 278 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 279 - protein, 106 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 280 - protein, 5 aa, synthetic construct
YYGMN

SEQ ID NO: 281 - protein, 17 aa, synthetic construct
WIDTTTGEPTYAQKFQG

SEQ ID NO: 282 - protein, 10 aa, synthetic construct
RGPYNWYFDV

SEQ ID NO: 283 - protein, 16 aa, synthetic construct
RSSKSLLHSNGNTYLY

SEQ ID NO: 284 - protein, 7 aa, synthetic construct
RMSNLVS

SEQ ID NO: 285 - protein, 9 aa, synthetic construct
LQHLEYPFT

SEQ ID NO: 286 - protein, 119 aa, synthetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 287 - protein, 112 aa, synthetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 288 - protein, 5 aa, synthetic construct
NYGMN

SEQ ID NO: 289 - protein, 17 aa, synthetic construct
WINTYTGEPTYADDFKG

SEQ ID NO: 290 - protein, 9 aa, synthetic construct
IGDSSPSDY

SEQ ID NO: 291 - protein, 11 aa, synthetic construct
KASQSVSNDVV

SEQ ID NO: 292 - protein, 7 aa, synthetic construct
YASNRYT

SEQ ID NO: 293 - protein, 9 aa, synthetic construct
QQDYTSPWT

SEQ ID NO: 294 - protein, 118 aa, synthetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 295 - protein, 107 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 296 - protein, 5 aa, synthetic construct
SYWIE

SEQ ID NO: 297 - protein, 17 aa, synthetic construct
EILPGSGNTYYNERFKD

SEQ ID NO: 298 - protein, 14 aa, synthetic construct
RAAAYYSNPEWFAY

SEQ ID NO: 299 - protein, 12 aa, synthetic construct
TASSSVNSFYLH

SEQ ID NO: 300 - protein, 7 aa, synthetic construct
STSNLAS

SEQ ID NO: 301 - protein, 9 aa, synthetic construct
HQYHRSPYT

SEQ ID NO: 302 - protein, 123 aa, synthetic construct
QVQLVQSGAEVKKPGSSVKVSCKASGGTFSSYWIEWVRQAPGQGLEWMGEILPGSGNTYYNERFKDRVTITADESTSTAYMELSSLRSEDTAVYYCARRAAAYYSNPEWFAYWGQGTLVTVSS

SEQ ID NO: 303 - protein, 108 aa, synthetic construct
EIVLTQSPATLSLSPGERATLSCTASSSVNSFYLHWYQQKPGLAPRLLIYSTSNLASGIPDRFSGSGSGTDFTLTISRLEPEDFAVYYCHQYHRSPYTFGQGTKLEIK

SEQ ID NO: 304 - protein, 5 aa, synthetic construct
SYWMQ

SEQ ID NO: 305 - protein, 17 aa, synthetic construct
TIYPGDGDTTYTQKFQG

SEQ ID NO: 306 - protein, 10 aa, synthetic construct
YDAPGYAMDY

SEQ ID NO: 307 - protein, 11 aa, synthetic construct
RASQDINNYLA

SEQ ID NO: 308 - protein, 7 aa, synthetic construct
YTSTLHP

SEQ ID NO: 309 - protein, 9 aa, synthetic construct
LQYDNLLYT

SEQ ID NO: 310 - protein, 119 aa, synthetic construct
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 311 - protein, 107 aa, synthetic construct
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 312 - protein, 6 aa, synthetic construct
RDFAWN

SEQ ID NO: 313 - protein, 16 aa, synthetic construct
YISYNGNTRYQPSLKS

SEQ ID NO: 314 - protein, 7 aa, synthetic construct
ASRGFPY

SEQ ID NO: 315 - protein, 11 aa, synthetic construct
HSSQDINSNIG

SEQ ID NO: 316 - protein, 7 aa, synthetic construct
HGTNLDD

SEQ ID NO: 317 - protein, 9 aa, synthetic construct
VQYAQFPWT

SEQ ID NO: 318 - protein, 116 aa, synthetic construct
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 319 - protein, 107 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 320 - protein, 6 aa, synthetic construct
RDFAWN

SEQ ID NO: 321 - protein, 16 aa, synthetic construct
YISYNGNTRYQPSLKS

SEQ ID NO: 322 - protein, 7 aa, synthetic construct
ASRGFPY

SEQ ID NO: 323 - protein, 11 aa, synthetic construct
HSSQDINSNIG

SEQ ID NO: 324 - protein, 7 aa, synthetic construct
HGTNLDD

SEQ ID NO: 325 - protein, 9 aa, synthetic construct
VQYAQFPWT

SEQ ID NO: 326 - protein, 116 aa, synthetic construct; identical to SEQ ID NO: 318

SEQ ID NO: 327 - protein, 107 aa, synthetic construct; identical to SEQ ID NO: 319

SEQ ID NO: 328 - protein, 5 aa, synthetic construct
NYGVH

SEQ ID NO: 329 - protein, 16 aa, synthetic construct
VIWSGGNTDYNTPFTS

SEQ ID NO: 330 - protein, 11 aa, synthetic construct
ALTYYDYEFAY

SEQ ID NO: 331 - protein, 11 aa, synthetic construct
RASQSIGTNIH

SEQ ID NO: 332 - protein, 7 aa, synthetic construct
YASESIS

SEQ ID NO: 333 - protein, 9 aa, synthetic construct
QQNNNWPTT

SEQ ID NO: 334 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 335 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 336 - protein, 5 aa, synthetic construct
GYFMN

SEQ ID NO: 337 - protein, 17 aa, synthetic construct
RIHPYDGDTFYNQKFQG

SEQ ID NO: 338 - protein, 9 aa, synthetic construct
YDGSRAMDY

SEQ ID NO: 339 - protein, 15 aa, synthetic construct
KASQSVSFAGTSLMH

SEQ ID NO: 340 - protein, 7 aa, synthetic construct
RASNLEA

SEQ ID NO: 341 - protein, 9 aa, synthetic construct
QQSREYPYT

SEQ ID NO: 342 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 343 - protein, 111 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 344 - protein, 5 aa, synthetic construct
GYGLS

SEQ ID NO: 345 - protein, 17 aa, synthetic construct
MISSGGSYTYYADSVKG

SEQ ID NO: 346 - protein, 10 aa, synthetic construct
HGDDPAWFAY

SEQ ID NO: 347 - protein, 12 aa, synthetic construct
SVSSSISSNNLH

SEQ ID NO: 348 - protein, 7 aa, synthetic construct
GTSNLAS

SEQ ID NO: 349 - protein, 11 aa, synthetic construct
QQWSSYPYMYT

SEQ ID NO: 350 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 351 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 352 - protein, 5 aa, synthetic construct
NYWMN

SEQ ID NO: 353 - protein, 19 aa, synthetic construct
EIRLKSNNYTTHYAESVKG

SEQ ID NO: 354 - protein, 6 aa, synthetic construct
HYYFDY

SEQ ID NO: 355 - protein, 16 aa, synthetic construct
RSSKSLLHSNGITYFF

SEQ ID NO: 356 - protein, 7 aa, synthetic construct
QMSNLAS

SEQ ID NO: 357 - protein, 9 aa, synthetic construct
AQNLELPPT

SEQ ID NO: 358 - protein, 117 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 359 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 360 - protein, 5 aa, synthetic construct
SYWIG

SEQ ID NO: 361 - protein, 17 aa, synthetic construct
IIDPGDSRTRYSPSFQG

SEQ ID NO: 362 - protein, 11 aa, synthetic construct
GQLYGGTYMDG

SEQ ID NO: 363 - protein, 14 aa, synthetic construct
TGTSSDIGGYNSVS

SEQ ID NO: 364 - protein, 7 aa, synthetic construct
GVNNRPS

SEQ ID NO: 365 - protein, 11 aa, synthetic construct
SSYDIESATPV

SEQ ID NO: 366 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 367 - protein, 111 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 368 - protein, 5 aa, synthetic construct
AYNIH

SEQ ID NO: 369 - protein, 17 aa, synthetic construct
SFDPYDGGSSYNQKFKD

SEQ ID NO: 370 - protein, 7 aa, synthetic construct
GWYYFDY

SEQ ID NO: 371 - protein, 11 aa, synthetic construct
RASKSISKYLA

SEQ ID NO: 372 - protein, 7 aa, synthetic construct
SGSTLQS

SEQ ID NO: 373 - protein, 9 aa, synthetic construct
QQHDESPYT

SEQ ID NO: 374 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 375 - protein, 107 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 376 - protein, 11 aa, synthetic construct
GSIKSGSYYWG

SEQ ID NO: 377 - protein, 16 aa, synthetic construct
NIYYSGSTYYNPSLRS

SEQ ID NO: 378 - protein, 12 aa, synthetic construct
AREGSYPNQFDP

SEQ ID NO: 379 - protein, 11 aa, synthetic construct
RASQSVSSNLA

SEQ ID NO: 380 - protein, 7 aa, synthetic construct
GASTRAT

SEQ ID NO: 381 - protein, 9 aa, synthetic construct
QQYHSFPFT

SEQ ID NO: 382 - protein, 120 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 383 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 384 - protein, 5 aa, synthetic construct
SFGMH

SEQ ID NO: 385 - protein, 11 aa, synthetic construct
YISSDSSAIYY

SEQ ID NO: 386 - protein, 12 aa, synthetic construct
GRENIYYGSRLD

SEQ ID NO: 387 - protein, 7 aa, synthetic construct
KASQNVD

SEQ ID NO: 388 - protein, 11 aa, synthetic construct
SASYRYSGVPD

SEQ ID NO: 389 - protein, 12 aa, synthetic construct
QQYNNYPFTFGS

SEQ ID NO: 390 - protein, 122 aa, synthetic construct
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 391 - protein, 107 aa, synthetic construct
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 392 - protein, 10 aa, synthetic construct
SYWMQWVRQA

SEQ ID NO: 393 - protein, 11 aa, synthetic construct
TIYPGDGDTRY

SEQ ID NO: 394 - protein, 12 aa, synthetic construct
RGIPRLWYFDVM

SEQ ID NO: 395 - protein, 10 aa, synthetic construct
ITCRASQDIS

SEQ ID NO: 396 - protein, 11 aa, synthetic construct
YTSRLHSGVPS

SEQ ID NO: 397 - protein, 12 aa, synthetic construct
QQGNTLPPFTGG

SEQ ID NO: 398 - protein, 122 aa, synthetic construct; identical to SEQ ID NO: 390

SEQ ID NO: 399 - protein, 107 aa, synthetic construct; identical to SEQ ID NO: 391

SEQ ID NO: 400 - protein, 10 aa, synthetic construct
SYGMSWVRQA

SEQ ID NO: 401 - protein, 10 aa, synthetic construct
INSGGSNTYY

SEQ ID NO: 402 - protein, 9 aa, synthetic construct
HDGGAMDYW

SEQ ID NO: 403 - protein, 14 aa, synthetic construct
ITCRASESIYSYLA

SEQ ID NO: 404 - protein, 7 aa, synthetic construct
NTKTLPE

SEQ ID NO: 405 - protein, 11 aa, synthetic construct
HHYGTPPWTFG

SEQ ID NO: 406 - protein, 117 aa, synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 407 - protein, 108 aa, synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 408 - protein, 10 aa, synthetic construct
SFGMHWVRQA

SEQ ID NO: 409 - protein, 20 aa, synthetic construct
ISSGSGTIYYADTVKGRFTI

SEQ ID NO: 410 - protein, 12 aa, synthetic construct
HGYRYEGFDYWG

SEQ ID NO: 411 - protein, 14 aa, synthetic construct
ITCKASQNVDTNVA

SEQ ID NO: 412 - protein, 11 aa, synthetic construct
SASYRYSGVPS

SEQ ID NO: 413 - protein, 12 aa, synthetic construct
QQYNNYPFTFGQ

SEQ ID NO: 414 - protein, 119 aa, synthetic construct
EVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 415 - protein, 107 aa, synthetic construct
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIK

SEQ ID NO: 416 - protein, 5 aa, synthetic construct
NYVMH

SEQ ID NO: 417 - protein, 17 aa, synthetic construct
YINPYNDDVKYNEKFKG

SEQ ID NO: 418 - protein, 13 aa, synthetic construct
WGYYGSPLYYFDY

SEQ ID NO: 419 - protein, 10 aa, synthetic construct
RASSRLIYMH

SEQ ID NO: 420 - protein, 7 aa, synthetic construct
ATSNLAS

SEQ ID NO: 421 - protein, 9 aa, synthetic construct
QQWNSNPPT

SEQ ID NO: 422 - protein, 122 aa, synthetic construct
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 423 - protein, 106 aa, synthetic construct
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 424 - protein, 5 aa, synthetic construct
NYVMH

SEQ ID NO: 425 - protein, 17 aa, synthetic construct
YINPYNDDVKYNEKFKG

SEQ ID NO: 426 - protein, 13 aa, synthetic construct
WGYYGSPLYYFDY

SEQ ID NO: 427 - protein, 10 aa, synthetic construct
RASSRLIYMH

SEQ ID NO: 428 - protein, 7 aa, synthetic construct
ATSNLAS

SEQ ID NO: 429 - protein, 9 aa, synthetic construct
QQWNSNPPT

SEQ ID NO: 430 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 431 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 432 - protein, 10 aa, synthetic construct
GYSFTSYTIH

SEQ ID NO: 433 - protein, 17 aa, synthetic construct
YINPNSRNTDYAQKFQG

SEQ ID NO: 434 - protein, 12 aa, synthetic construct
YSGSTPYWYFDV

SEQ ID NO: 435 - protein, 10 aa, synthetic construct
RASSSVSYMN

SEQ ID NO: 436 - protein, 7 aa, synthetic construct
ATSNLAS

SEQ ID NO: 437 - protein, 9 aa, synthetic construct
QQWSSNPLT

SEQ ID NO: 438 - protein, 121 aa, synthetic construct
EVQLVQSGAEVKKPGSSVKVSCKASGYSFTSYTIHWVRQAPGQGLEWMGYINPNSRNTDYAQKFQGRVTLTADKSTSTAYMELSSLRSEDTAVYYCARYSGSTPYWYFDVWGQGTTVTVSS

SEQ ID NO: 439 - protein, 107 aa, synthetic construct; identical to SEQ ID NO: 231

SEQ ID NO: 440 - protein, 10 aa, synthetic construct
GYTFSSYWMH

SEQ ID NO: 441 - protein, 17 aa, synthetic construct
LIHPDSGSTNYNEMFKN

SEQ ID NO: 442 - protein, 7 aa, synthetic construct
GGRLYFD

SEQ ID NO: 443 - protein, 16 aa, synthetic construct
RSSQSLVHSNGDTYLR

SEQ ID NO: 444 - protein, 7 aa, synthetic construct
KVSNRFS

SEQ ID NO: 445 - protein, 9 aa, synthetic construct
SQSTHVPYT

SEQ ID NO: 446 - protein, 117 aa, synthetic construct
EVQLVQSGAEVKKPGSSVKVSCKASGYTFSSYWMHWVRQAPGQGLEWIGLIHPDSGSTNYNEMFKNRATLTVDRSTSTAYVELSSLRSEDTAVYFCAGGGRLYFDYWGQGTTVTVSS

SEQ ID NO: 447 - protein, 112 aa, synthetic construct
DVVMTQSPLSLPVTPGEPASISCRSSQSLVHSNGDTYLRWYLQKPGQSPQLLIYKVSNRFSGVPDRFSGSGSGTDFTLKISRVEAEDVGVYYCSQSTHVPYTFGGGTKVEIK

SEQ ID NO: 448 - protein, 10 aa, synthetic construct
GYTFSSYWMH

SEQ ID NO: 449 - protein, 17 aa, synthetic construct
LIHPESGSTNYNEMFKN

SEQ ID NO: 450 - protein, 8 aa, synthetic construct
GGRLYFDY

SEQ ID NO: 451 - protein, 16 aa, synthetic construct
RSSQSLVHSNQDTYLR

SEQ ID NO: 452 - protein, 7 aa, synthetic construct
KVSNRFS

SEQ ID NO: 453 - protein, 9 aa, synthetic construct
SQSTHVPYT

SEQ ID NO: 454 - protein, 117 aa, synthetic construct
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 455 - protein, 113 aa, synthetic construct
DIVMTQSPLSLPVTPGEPASISCRSSQSLVHSNQDTYLRWYLQKPGQSPQLLIYKVSNRFSGVPDRFSGSGSGTDFTLKKISRVEAEDVGVYYCSQSTHVPYTFGGGTKVEIK

SEQ ID NO: 456 - protein, 12 aa, synthetic construct
TGYSITSGYSWH

SEQ ID NO: 457 - protein, 16 aa, synthetic construct
YIHSSGSTNYNPSLKS

SEQ ID NO: 458 - protein, 7 aa, synthetic construct
YDDYFEY

SEQ ID NO: 459 - protein, 12 aa, synthetic construct
KASQNVGFNVAW

SEQ ID NO: 460 - protein, 7 aa, synthetic construct
SASYRYS

SEQ ID NO: 461 - protein, 9 aa, synthetic construct
QQYNWYPFT

SEQ ID NO: 462 - protein, 116 aa, synthetic construct; identical to SEQ ID NO: 230

SEQ ID NO: 463 - protein, 108 aa, synthetic construct
DIQMTQSPSSLSASVGDRVTITCKASQNVGGFNVAWYQQKPGKSPKALIYSASYRYSGVPSRFSGSGSGTDFTLTISSLQPEDFAEYFCQQYNWYPFTFGQGTKLEIK

SEQ ID NO: 464 - protein, 5 aa, synthetic construct
NYDIN

SEQ ID NO: 465 - protein, 20 aa, synthetic construct
WIGWIFPGDDSTQYNEKFKG

SEQ ID NO: 466 - protein, 9 aa, synthetic construct
QTTGTWFAY

SEQ ID NO: 467 - protein, 11 aa, synthetic construct
RASQSISDYLY

SEQ ID NO: 468 - protein, 7 aa, synthetic construct
YASQSIS

SEQ ID NO: 469 - protein, 9 aa, synthetic construct
CQNGHSFPL

SEQ ID NO: 470 - protein, 118 aa, synthetic construct
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSS

SEQ ID NO: 471 - protein, 107 aa, synthetic construct
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELK

SEQ ID NO: 472 - protein, 119 aa, synthetic construct
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 473 - protein, 109 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 474 - protein, 5 aa, synthetic construct
IYNVH

SEQ ID NO: 475 - protein, 17 aa, synthetic construct
TIFPGNGDTSYNQKFKD

SEQ ID NO: 476 - protein, 10 aa, synthetic construct
WDDGNVGFAH

SEQ ID NO: 477 - protein, 11 aa, synthetic construct
RASENINNYLT

SEQ ID NO: 478 - protein, 7 aa, synthetic construct
HAKTLAE

SEQ ID NO: 479 - protein, 9 aa, synthetic construct
QHHYGTPPT

SEQ ID NO: 480 - protein, 119 aa, synthetic construct
QVQLQQPGAELVKPGASVKMSCKASGYTFTIYNVHWIKQTPGQGLEWMGTIFPGNGDTSYNQKFKDKATLTTDKSSKTAYMQLNSLTSEDSAVYYCARWDDGNVGFAHWGQGTLVTVSA

SEQ ID NO: 481 - protein, 107 aa, synthetic construct
DIQMTQSPASLSASVGETVTITCRASENINNYLTWFQQKQGKSPQLLVYHAKTLAEGVPSRFSGSGSGTQFSLKINSLQPEDFGSYYCQHHYGTPPTFGGGTKLEIK

SEQ ID NO: 482 - protein, 119 aa, synthetic construct
EVQLVQSGAEVKKPGASVKVSCKASGYTFTIYNVHWVRQAPGQGLEWMGTIFPGNGDTSYNQKFKDKVTMTTDTSTSTAYMELSSLRSEDTAVYYCARWDDGNVGFAHWGQGTLVTVSS

SEQ ID NO: 483 - protein, 107 aa, synthetic construct
DIQMTQSPSSLSASVGDRVTITCRASENINNYLTWFQQKQGKSPQLLIYHAKTLAEGVPSRFSGSGSGTDFTLTISSLQPEDFATYYCQHHYGTPPTFGGGTKVEIK

SEQ ID NO: 484 - protein, 119 aa, synthetic construct
EVQLVQSGAEVKKPGASVKVSCKASGYTFTIYNVHWIRQAPGQGLEWMGTIFPGNGDTSYNQKFKDRATLTTDKSTKTAYMELRSLRSDDTAVYYCARWDDGNVGFAHWGQGTLVTVSS

SEQ ID NO: 485 - protein, 107 aa, synthetic construct
DIQMTQSPSSLSASVGDRVTITCRASENINNYLTWFQQKPGKAPKLLVYHAKTLAEGVPSRFSGSGSGTQFTLTISSLQPEDFATYYCQHHYGTPPTFGQGTKLEIK

SEQ ID NO: 486 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 488 - protein, 445 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 489 - protein, 217 aa, synthetic construct
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 490 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 491 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 449 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 7 aa, synthetic construct
TSNMGVG

SEQ ID NO: 495 - protein, 16 aa, synthetic construct
HIWWDDDKYYSPSLKS

SEQ ID NO: 496 - protein, 10 aa, synthetic construct
SNYGYAWFAY

SEQ ID NO: 497 - protein, 11 aa, synthetic construct
KASQDIYPYLN

SEQ ID NO: 498 - protein, 7 aa, synthetic construct
RTNRLLD

SEQ ID NO: 499 - protein, 9 aa, synthetic construct
LQYDEFPLT

SEQ ID NO: 500 - protein, 120 aa, synthetic construct
QITLKESGPTLVKPTQTLTLTCTFSGFSLSTSNMGVGWIRQPPGKALEWLAHIWWDDDKYYSPSLKSRLTITKDTSKNQVVLTMTNMDPVDTATYYCVRSNYGYAWFAYWGQGTLVTVSS

SEQ ID NO: 501 - protein, 107 aa, synthetic construct
DIQMTQSPSSLSASVGDRVTITCKASQDIYPYLNWFQQKPGKAPKTLIYRTNRLLDGVPSRFSGSGSGTDFTFTISSLQPEDIATYYCLQYDEFPLTFGAGTKLEIK

SEQ ID NO: 502 - protein, 5 aa, synthetic construct
DYAVH

SEQ ID NO: 503 - protein, 17 aa, synthetic construct
VISTYNDYTYNNQDFKG

SEQ ID NO: 504 - protein, 10 aa, synthetic construct
GNSYFYALDY

SEQ ID NO: 505 - protein, 15 aa, synthetic construct
RASESVDSYGKSFMH

SEQ ID NO: 506 - protein, 7 aa, synthetic construct
RASNLES

SEQ ID NO: 507 - protein, 9 aa, synthetic construct
QQSNEDPWT

SEQ ID NO: 508 - protein, 119 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 509 - protein, 111 aa, synthetic construct
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 510 - protein, 5 aa, synthetic construct
RYWMS

SEQ ID NO: 511 - protein, 17 aa, synthetic construct
DLNPDSSAINYVDSVKG

SEQ ID NO: 512 - protein, 11 aa, synthetic construct
ITTLVPYTMDF

SEQ ID NO: 513 - protein, 11 aa, synthetic construct
ITNTDIDDDMN

SEQ ID NO: 514 - protein, 7 aa, synthetic construct
EGNGLRP

SEQ ID NO: 515 - protein, 9 aa, synthetic construct
LQSDNLPLT

SEQ ID NO: 516 - protein, 120 aa, synthetic construct
EVQLVESGGGLVQPGGSLRLSCAASGFDFSRYWMSWVRQAPGKGLEWIGDLNPDSSAINYVDSVKGRFTISRDNAKNSLYLQMNSLRAEDTAVYYCTLITTLVPYTMDFWGQGTSVTVSS

SEQ ID NO: 517 - protein, 107 aa, synthetic construct
ETTLTQSPAFMSATPGDKVNISCITNTDIDDDMNWYQQKPGEAAILLISEGNGLRPGIPPRFSGSGYGTDFTLTINNIESEDAAYYFCLQSDNLPLTFGSGTKLEIK

SEQ ID NO: 518 - protein, 5 aa, synthetic construct
DYYMH

SEQ ID NO: 519 - protein, 17 aa, synthetic construct
WIDPENGDTEYGPKFQG

SEQ ID NO: 520 - protein, 11 aa, synthetic construct
HNAHYGTWFAY

SEQ ID NO: 521 - protein, 16 aa, synthetic construct
RSSQSLLHSSGNTYLE

SEQ ID NO: 522 - protein, 7 aa, synthetic construct
KISTRFS

SEQ ID NO: 523 - protein, 9 aa, synthetic construct
FQGSHVPYT

SEQ ID NO: 524 - protein, 120 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 525 - protein, 112 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 526 - protein, 5 aa, synthetic construct
DYNVN

SEQ ID NO: 527 - protein, 17 aa, synthetic construct
VINPKYGTTRYNQKFKG

SEQ ID NO: 528 - protein, 7 aa, synthetic construct
GLNAWDY

SEQ ID NO: 529 - protein, 11 aa, synthetic construct
GASENIYGALN

SEQ ID NO: 530 - protein, 7 aa, synthetic construct
GATNLED

SEQ ID NO: 531 - protein, 9 aa, synthetic construct
QNVLTTPYT

SEQ ID NO: 532 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 533 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 534 - protein, 5 aa, synthetic construct
GYFMN

SEQ ID NO: 535 - protein, 17 aa, synthetic construct
LINPYNGDSFYNQKFKG

SEQ ID NO: 536 - protein, 8 aa, synthetic construct
GLRRDFDY

SEQ ID NO: 537 - protein, 16 aa, synthetic construct
KSSQSLLDSDGKTYLN

SEQ ID NO: 538 - protein, 7 aa, synthetic construct
LVSELDS

SEQ ID NO: 539 - protein, 9 aa, synthetic construct
WQGTHFPRT

SEQ ID NO: 540 - protein, 117 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 541 - protein, 112 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 542 - protein, 5 aa, synthetic construct
DFGMN

SEQ ID NO: 543 - protein, 17 aa, synthetic construct
WINTFTGEPSYGNVFKG

SEQ ID NO: 544 - protein, 10 aa, synthetic construct
RHGNGNVFDS

SEQ ID NO: 545 - protein, 11 aa, synthetic construct
RASQSIGSNIH

SEQ ID NO: 546 - protein, 7 aa, synthetic construct
YTSESIS

SEQ ID NO: 547 - protein, 9 aa, synthetic construct
QQSNSWPLT

SEQ ID NO: 548 - protein, 119 aa, synthetic construct
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 549 - protein, 108 aa, synthetic construct
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKR

SEQ ID NO: 550 - protein, 5 aa, synthetic construct
TAAIS

SEQ ID NO: 551 - protein, 17 aa, synthetic construct
GIIPIFGKAHYAQKFQG

SEQ ID NO: 552 - protein, 14 aa, synthetic construct
KFHFVSGSPFGMDV

SEQ ID NO: 553 - protein, 11 aa, synthetic construct
RASQSVSSYLA

SEQ ID NO: 554 - protein, 7 aa, synthetic construct
DASNRAT

SEQ ID NO: 555 - protein, 8 aa, synthetic construct
QQRSNWPT

SEQ ID NO: 556 - protein, 123 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 557 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 558 - protein, 5 aa, synthetic construct
SYAIS

SEQ ID NO: 559 - protein, 17 aa, synthetic construct
GIIPIFGTANYAQKFQG

SEQ ID NO: 560 - protein, 21 aa, synthetic construct
APLRFLEWSTQDHYYYYYMDV

SEQ ID NO: 561 - protein, 11 aa, synthetic construct
QGDSLRSYYAT

SEQ ID NO: 562 - protein, 7 aa, synthetic construct
GENKRPS

SEQ ID NO: 563 - protein, 11 aa, synthetic construct
KSRDGSGQHLV

SEQ ID NO: 564 - protein, 130 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 565 - protein, 108 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 566 - protein, 5 aa, synthetic construct
SYWIN

SEQ ID NO: 567 - protein, 17 aa, synthetic construct
NIYPSDSYTNYNQKFKD

SEQ ID NO: 568 - protein, 9 aa, synthetic construct
SWRGNSFDY

SEQ ID NO: 569 - protein, 17 aa, synthetic construct
KSSQSLLNSGNQKNYLT

SEQ ID NO: 570 - protein, 7 aa, synthetic construct
WASTRES

SEQ ID NO: 571 - protein, 9 aa, synthetic construct
QNDYSYPFT

SEQ ID NO: 572 - protein, 118 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 573 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 574 - protein, 5 aa, synthetic construct
NYGMN

SEQ ID NO: 575 - protein, 17 aa, synthetic construct
WINTNTGEPTYAEEFKG

SEQ ID NO: 576 - protein, 9 aa, synthetic construct
LGFGNAMDY

SEQ ID NO: 577 - protein, 17 aa, synthetic construct
KSSQSLLNSGNQKNYLT

SEQ ID NO: 578 - protein, 7 aa, synthetic construct
WASTRES

SEQ ID NO: 579 - protein, 9 aa, synthetic construct
QNDYSYPLT

SEQ ID NO: 580 - protein, 118 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 581 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 582 - protein, 5 aa, synthetic construct
SYNMN

SEQ ID NO: 583 - protein, 17 aa, synthetic construct
YISSSSSTIYYADSVKG

SEQ ID NO: 584 - protein, 8 aa, synthetic construct
AYYYGMDV

SEQ ID NO: 585 - protein, 11 aa, synthetic construct
RASQGISGWLA

SEQ ID NO: 586 - protein, 7 aa, synthetic construct
AASTLQS

SEQ ID NO: 587 - protein, 9 aa, synthetic construct
QQANSFPPT

SEQ ID NO: 588 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 589 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 590 - protein, 5 aa, synthetic construct
SYGMH

SEQ ID NO: 591 - protein, 17 aa, synthetic construct
VIWYDGSNQYYADSVKG

SEQ ID NO: 592 - protein, 11 aa, synthetic construct
GLTSGRYGMDV

SEQ ID NO: 593 - protein, 16 aa, synthetic construct
RSSQSLLLSHGFNYLD

SEQ ID NO: 594 - protein, 7 aa, synthetic construct
LGSSRAS

SEQ ID NO: 595 - protein, 9 aa, synthetic construct
MQPLQIPWT

SEQ ID NO: 596 - protein, 120 aa, synthetic construct
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 597 - protein, 112 aa, synthetic construct
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 598 - protein, 5 aa, synthetic construct
SGYWN

SEQ ID NO: 599 - protein, 16 aa, synthetic construct
YISDSGITYYNPSLKS

SEQ ID NO: 600 - protein, 11 aa, synthetic construct
RTLATYYAMDY

SEQ ID NO: 601 - protein, 16 aa, synthetic construct
RASQSLVHSDGNTYLH

SEQ ID NO: 602 - protein, 7 aa, synthetic construct
RVSNRFS

SEQ ID NO: 603 - protein, 9 aa, synthetic construct
SQSTHVPPT

SEQ ID NO: 604 - protein, 119 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 605 - protein, 113 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKR

SEQ ID NO: 606 - protein, 5 aa, synthetic construct
NYAMS

SEQ ID NO: 607 - protein, 17 aa, synthetic construct
SISGSGDYTYYTDSVKG

SEQ ID NO: 608 - protein, 9 aa, synthetic construct
SPWGYYLDS

SEQ ID NO: 609 - protein, 11 aa, synthetic construct
RASQGISSRLA

SEQ ID NO: 610 - protein, 7 aa, synthetic construct
AASSLQS

SEQ ID NO: 611 - protein, 9 aa, synthetic construct
QQYNSYPYT

SEQ ID NO: 612 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 613 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 614 - protein, 5 aa, synthetic construct
DHAIH

SEQ ID NO: 615 - protein, 17 aa, synthetic construct
YFSPGNDDIKYNEKFRG

SEQ ID NO: 616 - protein, 6 aa, synthetic construct
SLSTPY

SEQ ID NO: 617 - protein, 17 aa, synthetic construct
KSSQSLLNRGNHKNYLT

SEQ ID NO: 618 - protein, 7 aa, synthetic construct
WASTRES

SEQ ID NO: 619 - protein, 9 aa, synthetic construct
QNDYTYPYT

SEQ ID NO: 620 - protein, 115 aa, synthetic construct
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 621 - protein, 113 aa, synthetic construct
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 622 - protein, 5 aa, synthetic construct
SYNMH

SEQ ID NO: 623 - protein, 17 aa, synthetic construct
AIYPGNGDTSYNQKFKG

SEQ ID NO: 624 - protein, 12 aa, synthetic construct
STYYGGDWYFNV

SEQ ID NO: 625 - protein, 10 aa, synthetic construct
RASSSVSYIH

SEQ ID NO: 626 - protein, 7 aa, synthetic construct
ATSNLAS

SEQ ID NO: 627 - protein, 9 aa, synthetic construct
QQWTSNPPT

SEQ ID NO: 628 - protein, 121 aa, synthetic construct [sequence omitted]

SEQ ID NO: 629 - protein, 106 aa, synthetic construct [sequence omitted]

SEQ ID NO: 630 - protein, 5 aa, synthetic construct
DTYIH

SEQ ID NO: 631 - protein, 17 aa, synthetic construct
RIYPTNGYTRYADSVKG

SEQ ID NO: 632 - protein, 11 aa, synthetic construct
WGGDGFYAMDY

SEQ ID NO: 633 - protein, 11 aa, synthetic construct
RASQDVNTAVA

SEQ ID NO: 634 - protein, 7 aa, synthetic construct
SASFLYS

SEQ ID NO: 635 - protein, 9 aa, synthetic construct
QQHYTTPPT

SEQ ID NO: 636 - protein, 120 aa, synthetic construct [sequence omitted]

SEQ ID NO: 637 - protein, 107 aa, synthetic construct [sequence omitted]

SEQ ID NO: 638 - protein, 5 aa, synthetic construct
SYWIE

SEQ ID NO: 639 - protein, 17 aa, synthetic construct
EILPGGGDTNYNEIFKG

SEQ ID NO: 640 - protein, 8 aa, synthetic construct
RVPIRLDY

SEQ ID NO: 641 - protein, 15 aa, synthetic construct
KASQSVDYEGDSFLN

SEQ ID NO: 642 - protein, 7 aa, synthetic construct
AASNLES

SEQ ID NO: 643 - protein, 9 aa, synthetic construct
QQSNEDPLT

SEQ ID NO: 644 - protein, 117 aa, synthetic construct [sequence omitted]

SEQ ID NO: 645 - protein, 111 aa, synthetic construct [sequence omitted]

SEQ ID NO: 646 - protein, 5 aa, synthetic construct
DFAMS

SEQ ID NO: 647 - protein, 17 aa, synthetic construct
TIGRVAFHTYYPDSMKG

SEQ ID NO: 648 - protein, 11 aa, synthetic construct
HRGFDVGHFDF

SEQ ID NO: 649 - protein, 16 aa, synthetic construct
RSSETLVHSSGNTYLE

SEQ ID NO: 650 - protein, 7 aa, synthetic construct
RVSNRFS

SEQ ID NO: 651 - protein, 9 aa, synthetic construct
FQGSFNPLT

SEQ ID NO: 652 - protein, 120 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 653 - protein, 112 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 654 - protein, 6 aa, synthetic construct
NDYAWN

SEQ ID NO: 655 - protein, 16 aa, synthetic construct
YISYSGYTTYNPSLKS

SEQ ID NO: 656 - protein, 7 aa, synthetic construct
WTSGLDY

SEQ ID NO: 657 - protein, 11 aa, synthetic construct
KASDLIHNWLA

SEQ ID NO: 658 - protein, 7 aa, synthetic construct
GATSLET

SEQ ID NO: 659 - protein, 9 aa, synthetic construct
QQYWTTPFT

SEQ ID NO: 660 - protein, 116 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 661 - protein, 107 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 662 - protein, 6 aa, synthetic construct
SDYAWN

SEQ ID NO: 663 - protein, 16 aa, synthetic construct
YISNSGSTSYNPSLKS

SEQ ID NO: 664 - protein, 15 aa, synthetic construct
ERNYDYDDYYYAMDY

SEQ ID NO: 665 - protein, 17 aa, synthetic construct
KSSQSLLYRSNQKNYLA

SEQ ID NO: 666 - protein, 7 aa, synthetic construct
WASTRES

SEQ ID NO: 667 - protein, 9 aa, synthetic construct
QQYYNYPRT

SEQ ID NO: 668 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 669 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 670 - protein, 5 aa, synthetic construct
NYWMH

SEQ ID NO: 671 - protein, 17 aa, synthetic construct
ATYRGHSDTYYNQKFKG

SEQ ID NO: 672 - protein, 12 aa, synthetic construct
GAIYDGYDVLDN

SEQ ID NO: 673 - protein, 11 aa, synthetic construct
SASQDISNYLN

SEQ ID NO: 674 - protein, 7 aa, synthetic construct
YTSNLHS

SEQ ID NO: 675 - protein, 9 aa, synthetic construct
QQYRKLPWT

SEQ ID NO: 676 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 677 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 678 - protein, 5 aa, synthetic construct
AYTMH

SEQ ID NO: 679 - protein, 17 aa, synthetic construct
WIKPNNGLANYAQKFQG

SEQ ID NO: 680 - protein, 9 aa, synthetic construct
SEITTEFDY

SEQ ID NO: 681 - protein, 15 aa, synthetic construct
KSSESVDSYANSFLH

SEQ ID NO: 682 - protein, 7 aa, synthetic construct
RASTRES

SEQ ID NO: 683 - protein, 9 aa, synthetic construct
QQSKEDPLT

SEQ ID NO: 684 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 685 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 686 - protein, 6 aa, synthetic construct
SDFAWN

SEQ ID NO: 687 - protein, 16 aa, synthetic construct
YISYSGNTRYQPSLKS

SEQ ID NO: 688 - protein, 7 aa, synthetic construct
AGRGFPY

SEQ ID NO: 689 - protein, 11 aa, synthetic construct
HSSQDINSNIG

SEQ ID NO: 690 - protein, 7 aa, synthetic construct
HGTNLDD

SEQ ID NO: 691 - protein, 9 aa, synthetic construct
VQYAQFPWT

SEQ ID NO: 692 - protein, 116 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 693 - protein, 107 aa, synthetic construct; identical to SEQ ID NO: 319

SEQ ID NO: 694 - protein, 5 aa, synthetic construct
DYYMA

SEQ ID NO: 695 - protein, 17 aa, synthetic construct
SINYDGSSTYYVDSVKG

SEQ ID NO: 696 - protein, 8 aa, synthetic construct
DRGYYFDY

SEQ ID NO: 697 - protein, 16 aa, synthetic construct
RSSQSLVHSNGNTYLH

SEQ ID NO: 698 - protein, 7 aa, synthetic construct
KVSNRFS

SEQ ID NO: 699 - protein, 10 aa, synthetic construct
SQSTHVPPFT

SEQ ID NO: 700 - protein, 117 aa, synthetic construct
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 701 - protein, 113 aa, synthetic construct
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 702 - protein, 5 aa, synthetic construct
SYGMH

SEQ ID NO: 703 - protein, 17 aa, synthetic construct
VIWYDGSNQYYADSVKG

SEQ ID NO: 704 - protein, 11 aa, synthetic construct
GLTSGRYGMDV

SEQ ID NO: 705 - protein, 16 aa, synthetic construct
RSSQSLLLSHGFNYLD

SEQ ID NO: 706 - protein, 7 aa, synthetic construct
LGSSRAS

SEQ ID NO: 707 - protein, 9 aa, synthetic construct
MQPLQIPWT

SEQ ID NO: 708 - protein, 120 aa, synthetic construct; identical to SEQ ID NO: 596

SEQ ID NO: 709 - protein, 112 aa, synthetic construct; identical to SEQ ID NO: 597

SEQ ID NO: 710 - protein, 5 aa, synthetic construct
NAWMS

SEQ ID NO: 711 - protein, 17 aa, synthetic construct
YISSSGSTIYYADSVKG

SEQ ID NO: 712 - protein, 8 aa, synthetic construct
EGLWAFDY

SEQ ID NO: 713 - protein, 14 aa, synthetic construct
TGSSSNIGAGYVVH

SEQ ID NO: 714 - protein, 7 aa, synthetic construct
DNNKRPS

SEQ ID NO: 715 - protein, 11 aa, synthetic construct
AAWDDRLNGPV

SEQ ID NO: 716 - protein, 117 aa, synthetic construct
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 717 - protein, 111 aa, synthetic construct
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 718 - protein, 5 aa, synthetic construct
GYYWS

SEQ ID NO: 719 - protein, 16 aa, synthetic construct
EINHRGNTNDNPSLKS

SEQ ID NO: 720 - protein, 11 aa, synthetic construct
ERGYTYGNFDH

SEQ ID NO: 721 - protein, 11 aa, synthetic construct
RASQSVSRNLA

SEQ ID NO: 722 - protein, 7 aa, synthetic construct
GASTRAT

SEQ ID NO: 723 - protein, 9 aa, synthetic construct
QQYKTWPRT

SEQ ID NO: 724 - protein, 119 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 725 - protein, 107 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 726 - protein, 5 aa, synthetic construct
SYAMN

SEQ ID NO: 727 - protein, 17 aa, synthetic construct
TTSGSGASTYYADSVKG

SEQ ID NO: 728 - protein, 7 aa, synthetic construct
IWIAFDI

SEQ ID NO: 729 - protein, 12 aa, synthetic construct
RASQSVSSSYLA

SEQ ID NO: 730 - protein, 7 aa, synthetic construct
GASSRAT

SEQ ID NO: 731 - protein, 9 aa, synthetic construct
QQYGSSPYT

SEQ ID NO: 732 - protein, 116 aa, synthetic construct
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 733 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 734 - protein, 7 aa, synthetic construct
SFNYYWS

SEQ ID NO: 735 - protein, 16 aa, synthetic construct
YIYYSGSTYSNPSLKS

SEQ ID NO: 736 - protein, 9 aa, synthetic construct
GYNWNYFDY

SEQ ID NO: 737 - protein, 11 aa, synthetic construct
RASQSVDNNLV

SEQ ID NO: 738 - protein, 7 aa, synthetic construct
GASTRAT

SEQ ID NO: 739 - protein, 10 aa, synthetic construct [sequence omitted]

SEQ ID NO: 740 - protein, 119 aa, synthetic construct [sequence omitted]

SEQ ID NO: 741 - protein, 108 aa, synthetic construct [sequence omitted]

SEQ ID NO: 742 - protein, 5 aa, synthetic construct
TYWMH

SEQ ID NO: 743 - protein, 17 aa, synthetic construct
EIDPSDSYSNYNQKFKD

SEQ ID NO: 744 - protein, 11 aa, synthetic construct
NGGLGPAWFSY

SEQ ID NO: 745 - protein, 11 aa, synthetic construct
KASQYVGTAVA

SEQ ID NO: 746 - protein, 7 aa, synthetic construct
SASNRYT

SEQ ID NO: 747 - protein, 9 aa, synthetic construct
QQYSSYPWT

SEQ ID NO: 748 - protein, 120 aa, synthetic construct
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 749 - protein, 107 aa, synthetic construct
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 750 - protein, 5 aa, synthetic construct
SYAMS

SEQ ID NO: 751 - protein, 17 aa, synthetic construct
AISGSGTSTYYADSVKG

SEQ ID NO: 752 - protein, 13 aa, synthetic construct
VRYNWNHGDWFDP

SEQ ID NO: 753 - protein, 13 aa, synthetic construct
SGSSSNIGNNYVS

SEQ ID NO: 754 - protein, 7 aa, synthetic construct
ENYNRPA

SEQ ID NO: 755 - protein, 11 aa, synthetic construct
SSWDDSLNYWV

SEQ ID NO: 756 - protein, 122 aa, synthetic construct
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 757 - protein, 110 aa, synthetic construct
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 758 - protein, 5 aa, synthetic construct
SYGMS

SEQ ID NO: 759 - protein, 17 aa, synthetic construct
TISSGGSYKYYVDSVKG

SEQ ID NO: 760 - protein, 11 aa, synthetic construct
HPDYDGVWFAY

SEQ ID NO: 761 - protein, 10 aa, synthetic construct
SVSSSVFYVH

SEQ ID NO: 762 - protein, 7 aa, synthetic construct
DTSKLAS

SEQ ID NO: 763 - protein, 9 aa, synthetic construct
QQWNSNPPT

SEQ ID NO: 764 - protein, 120 aa, synthetic construct
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 765 - protein, 107 aa, synthetic construct
DIQMTQSPSSLSASVGDRVTITCSVSSSVFYVHWYQQKPGKAPKLLIYDTSKLASSGVPSRFSGSGSGTDFTFTISSLQPEDIATYYCQQWNSNPPTFGGGTKVEIK

SEQ ID NO: 766 - protein, 5 aa, synthetic construct
SYGMS

SEQ ID NO: 767 - protein, 17 aa, synthetic construct
TISSGGSYTYYPDSVKG

SEQ ID NO: 768 - protein, 11 aa, synthetic construct
HPDYDGVWFAY

SEQ ID NO: 769 - protein, 10 aa, synthetic construct
SVSSSVFYVH

SEQ ID NO: 770 - protein, 7 aa, synthetic construct
DTSKLAS

SEQ ID NO: 771 - protein, 9 aa, synthetic construct
QQWNSNPPT

SEQ ID NO: 772 - protein, 120 aa, synthetic construct
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 773 - protein, 105 aa, synthetic construct
QIVLTQSPAIMASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 774 - protein, 5 aa, synthetic construct
SYYMH

SEQ ID NO: 775 - protein, 17 aa, synthetic construct
IINPSGGSTSYAQKFQG

SEQ ID NO: 776 - protein, 16 aa, synthetic construct
DGVLRYFDWLLDYYYY

SEQ ID NO: 777 - protein, 11 aa, synthetic construct
RASQSVGSYLA

SEQ ID NO: 778 - protein, 7 aa, synthetic construct
DASNRAT

SEQ ID NO: 779 - protein, 8 aa, synthetic construct
QQRANVFT

SEQ ID NO: 780 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 781 - protein, 106 aa, synthetic construct
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 782 - protein, 5 aa, synthetic construct
SYYMH

SEQ ID NO: 783 - protein, 17 aa, synthetic construct
IINPSGGSTSYAQKFQG

SEQ ID NO: 784 - protein, 19 aa, synthetic construct
DAELRHFDHLLDYHYYMDV

SEQ ID NO: 785 - protein, 11 aa, synthetic construct
RASQSVGSYLA

SEQ ID NO: 786 - protein, 7 aa, synthetic construct
DASNRAT

SEQ ID NO: 787 - protein, 8 aa, synthetic construct
QQRAQEFT

SEQ ID NO: 788 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 789 - protein, 106 aa, synthetic construct
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 790 - protein, 120 aa, synthetic construct
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 791 - protein, 107 aa, synthetic construct
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 792 - protein, 120 aa, synthetic construct
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 793 - protein, 107 aa, synthetic construct; identical to SEQ ID NO: 791

SEQ ID NO: 794 - protein, 5 aa, synthetic construct
NYYMA

SEQ ID NO: 795 - protein, 17 aa, synthetic construct
SITKGGGNTYYRDSVKG

SEQ ID NO: 796 - protein, 14 aa, synthetic construct
QVTIAAVSTSYFDS

SEQ ID NO: 797 - protein, 15 aa, synthetic construct
KTNQKVDYYGNSYVY

SEQ ID NO: 798 - protein, 7 aa, synthetic construct
LASNLAS

SEQ ID NO: 799 - protein, 9 aa, synthetic construct
QQSRNLPYT

SEQ ID NO: 800 - protein, 123 aa, synthetic construct
EVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 801 - protein, 110 aa, synthetic construct
DIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELK

SEQ ID NO: 802 - protein, 5 aa, synthetic construct
DYYMK

SEQ ID NO: 803 - protein, 17 aa, synthetic construct
DIIPSNGATFYNQKFKG

SEQ ID NO: 804 - protein, 11 aa, synthetic construct
SHLLRASWFAY

SEQ ID NO: 805 - protein, 17 aa, synthetic construct
KSSQSLLNSGNQKNYLT

SEQ ID NO: 806 - protein, 7 aa, synthetic construct
WASTRES

SEQ ID NO: 807 - protein, 9 aa, synthetic construct
QNDYSYPYT

SEQ ID NO: 808 - protein, 120 aa, synthetic construct
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 809 - protein, 113 aa, synthetic construct
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 810 - protein, 5 aa, synthetic construct
DYEMH

SEQ ID NO: 811 - protein, 20 aa, synthetic construct
WIGGIDPETGGTAYNQKFKG

SEQ ID NO: 812 - protein, 6 aa, synthetic construct
YYSFAY

SEQ ID NO: 813 - protein, 16 aa, synthetic construct
RSSQSIVHSNGNTYLQ

SEQ ID NO: 814 - protein, 7 aa, synthetic construct
KVSNRFS

SEQ ID NO: 815 - protein, 9 aa, synthetic construct
FQVSHVPYT

SEQ ID NO: 816 - protein, 115 aa, synthetic construct
EVQLVQSGAEVKKPGATVKISCKVSGYTFTDYEMHWVQQAPGKGLEWMGGIDPETGGTAYNQKFKGRVTLTADKSTDTAYMELSSLRSEDTAVYYCGRYYSFAYWGQGTLVTVSS

SEQ ID NO: 817 - protein, 112 aa, synthetic construct
DVVMTQSPLSLPVTLGQPASISCRSSQSIVHSNANTYLQWFQQRPGQSPRLLIYKVSNRFSGVPDRFSGSGSGTDFTLKISRVEAEDVGVYYCFQVSHVPYTFGQGTKLEIK

SEQ ID NO: 818 - protein, 5 aa, synthetic construct
DYNVN

SEQ ID NO: 819 - protein, 17 aa, synthetic construct
VINPKYGTTRYNQKFKG

SEQ ID NO: 820 - protein, 7 aa, synthetic construct
GLNAWDY

SEQ ID NO: 821 - protein, 11 aa, synthetic construct
GASENIYGALN

SEQ ID NO: 822 - protein, 7 aa, synthetic construct
GATNLED

SEQ ID NO: 823 - protein, 9 aa, synthetic construct
QNVLTTPYT

SEQ ID NO: 824 - protein, 116 aa, synthetic construct; identical to SEQ ID NO: 532

SEQ ID NO: 825 - protein, 107 aa, synthetic construct; identical to SEQ ID NO: 533

SEQ ID NO: 826 - protein, 5 aa, synthetic construct
GYFMN

SEQ ID NO: 827 - protein, 17 aa, synthetic construct
LINPYNGDSFYNQKFKG

SEQ ID NO: 828 - protein, 8 aa, synthetic construct
GLRRDFDY

SEQ ID NO: 829 - protein, 16 aa, synthetic construct
KSSQSLLDSDGKTYLN

SEQ ID NO: 830 - protein, 7 aa, synthetic construct
LVSELDS

SEQ ID NO: 831 - protein, 9 aa, synthetic construct
WQGTHFPRT

SEQ ID NO: 832 - protein, 117 aa, synthetic construct; identical to SEQ ID NO: 540

SEQ ID NO: 833 - protein, 112 aa, synthetic construct; identical to SEQ ID NO: 541

SEQ ID NO: 834 - protein, 5 aa, synthetic construct
TAAIS

SEQ ID NO: 835 - protein, 17 aa, synthetic construct
GIIPIFGKAHYAQKFQG

SEQ ID NO: 836 - protein, 14 aa, synthetic construct
KFHFVSGSPFGMDV

SEQ ID NO: 837 - protein, 11 aa, synthetic construct
RASQSVSSYLA

SEQ ID NO: 838 - protein, 7 aa, synthetic construct
DASNRAT

SEQ ID NO: 839 - protein, 8 aa, synthetic construct
QQRSNWPT

SEQ ID NO: 840 - protein, 123 aa, synthetic construct; identical to SEQ ID NO: 556

SEQ ID NO: 841 - protein, 106 aa, synthetic construct; identical to SEQ ID NO: 557

SEQ ID NO: 842 - protein, 9 aa, synthetic construct
GTFSSYAIS

SEQ ID NO: 843 - protein, 17 aa, synthetic construct
SIIPIFGTANYAQKFQG

SEQ ID NO: 844 - protein, 18 aa, synthetic construct
ARGPSEVGAILGYVWFDP

SEQ ID NO: 845 - protein, 16 aa, synthetic construct
RSSQSLLHSNGYNYLD

SEQ ID NO: 846 - protein, 7 aa, synthetic construct
LGSNRAS

SEQ ID NO: 847 - protein, 9 aa, synthetic construct
MQARRIPIT

SEQ ID NO: 848 - protein, 125 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 849 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 850 - protein, 12 aa, synthetic construct
GYTFTDHAIHWV

SEQ ID NO: 851 - protein, 10 aa, synthetic construct
FSPGNDDIKY

SEQ ID NO: 852 - protein, 8 aa, synthetic construct
KRSLSTPY

SEQ ID NO: 853 - protein, 12 aa, synthetic construct
QSLLNRGNHKNY

SEQ ID NO: 854 - protein, 7 aa, synthetic construct
WASTRES

SEQ ID NO: 855 - protein, 9 aa, synthetic construct
QNDYTYPYT

SEQ ID NO: 856 - protein, 115 aa, synthetic construct; identical to SEQ ID NO: 620

SEQ ID NO: 857 - protein, 113 aa, synthetic construct; identical to SEQ ID NO: 621

SEQ ID NO: 858 - protein, 5 aa, synthetic construct
NYDIN

SEQ ID NO: 859 - protein, 17 aa, synthetic construct
WIYPGDGSTKYNEKFKA

SEQ ID NO: 860 - protein, 8 aa, synthetic construct
GYEDAMDY

SEQ ID NO: 861 - protein, 11 aa, synthetic construct
KASQDINSYLS

SEQ ID NO: 862 - protein, 7 aa, synthetic construct
RANRLVD

SEQ ID NO: 863 - protein, 9 aa, synthetic construct
LQYDEFPLT

SEQ ID NO: 864 - protein, 117 aa, synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 865 - protein, 105 aa, synthetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVE

SEQ ID NO: 866 - protein, 5 aa, synthetic construct
NYGMN

SEQ ID NO: 867 - protein, 17 aa, synthetic construct
WINTYSGEPRYADDFKG

SEQ ID NO: 868 - protein, 9 aa, synthetic construct
DYGRWYFDV

SEQ ID NO: 869 - protein, 10 aa, synthetic construct
RASSSVSHMH

SEQ ID NO: 870 - protein, 7 aa, synthetic construct
ATSNLAS

SEQ ID NO: 871 - protein, 9 aa, synthetic construct
QQWSSTPRT

SEQ ID NO: 872 - protein, 118 aa, synthetic construct; identical to SEQ ID NO: 158

SEQ ID NO: 873 - protein, 106 aa, synthetic construct
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIK

SEQ ID NO: 874 - protein, 5 aa, synthetic construct
DYYIH

SEQ ID NO: 875 - protein, 17 aa, synthetic construct
YINPNSGYTNYAQKFQG

SEQ ID NO: 876 - protein, 12 aa, synthetic construct
YMWERVTGFFDF

SEQ ID NO: 877 - protein, 11 aa, synthetic construct
LASEDISDDLA

SEQ ID NO: 878 - protein, 7 aa, synthetic construct
TTSSLQS

SEQ ID NO: 879 - protein, 9 aa, synthetic construct
QQTYKFPPT

SEQ ID NO: 880 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 881 - protein, 107 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 882 - protein, 8 aa, synthetic construct
GFTFSNYA

SEQ ID NO: 883 - protein, 8 aa, synthetic construct
ISGSGDYT

SEQ ID NO: 884 - protein, 11 aa, synthetic construct
ARSPWGYYLDS

SEQ ID NO: 885 - protein, 6 aa, synthetic construct
QGISSR

SEQ ID NO: 886 - skipped

SEQ ID NO: 887 - protein, 9 aa, synthetic construct
QQYNSYPYT

SEQ ID NO: 888 - protein, 118 aa, synthetic construct; identical to SEQ ID NO: 612

SEQ ID NO: 889 - protein, 107 aa, synthetic construct; identical to SEQ ID NO: 613

SEQ ID NO: 890 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 891 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 892 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 893 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 894 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 895 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 896 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 897 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 898 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 899 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 900 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPCVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 901 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPCVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 902 - protein, 5 aa, synthetic construct
TAAIS

SEQ ID NO: 903 - protein, 17 aa, synthetic construct
GIIPIFGKAHYAQKFQG

SEQ ID NO: 904 - protein, 14 aa, synthetic construct
KFHFVSGSPFGMDV

SEQ ID NO: 905 - protein, 11 aa, synthetic construct
RASQSVSSYLA

SEQ ID NO: 906 - protein, 7 aa, synthetic construct
DASNRAT

SEQ ID NO: 907 - protein, 8 aa, synthetic construct
QQRSNWPT

SEQ ID NO: 908 - protein, 123 aa, synthetic construct; identical to SEQ ID NO: 556

SEQ ID NO: 909 - protein, 106 aa, synthetic construct; identical to SEQ ID NO: 557

SEQ ID NO: 910 - protein, 5 aa, synthetic construct
HYMMA

SEQ ID NO: 911 - protein, 17 aa, synthetic construct
RIGPSGGPTHYADSVKG

SEQ ID NO: 912 - protein, 19 aa, synthetic construct
YDSGYDYVAVAGPAEYFQH

SEQ ID NO: 913 - protein, 11 aa, synthetic construct
RASQSISTWLA

SEQ ID NO: 914 - protein, 7 aa, synthetic construct
KASNLHT

SEQ ID NO: 915 - protein, 9 aa, synthetic construct
QQYNSYSRT

SEQ ID NO: 916 - protein, 128 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 917 - protein, 107 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 918 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 919 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 920 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 921 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 922 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 923 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 924 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 925 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 926 - protein, 214 aa, synthetic construct; identical to SEQ ID NO: 923

SEQ ID NO: 927 - protein, 5 aa, synthetic construct
SGYWN

SEQ ID NO: 928 - protein, 16 aa, synthetic construct
YISDSGITYYNPSLKS

SEQ ID NO: 929 - protein, 11 aa, synthetic construct
RTLATYYAMDY

SEQ ID NO: 930 - protein, 16 aa, synthetic construct
RASQSLVHSDGNTYLH

SEQ ID NO: 931 - protein, 7 aa, synthetic construct
RVSNRFS

SEQ ID NO: 932 - protein, 9 aa, synthetic construct
SQSTHVPPT

SEQ ID NO: 933 - protein, 119 aa, synthetic construct; identical to SEQ ID NO: 604

SEQ ID NO: 934 - protein, 112 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIK

SEQ ID NO: 935 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 936 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 937 - protein, 219 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 938 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 939 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 940 - protein, 219 aa, synthetic construct; identical to SEQ ID NO: 937

SEQ ID NO: 941 - protein, 5 aa, synthetic construct
DYNVN

SEQ ID NO: 942 - protein, 17 aa, synthetic construct
VINPKYGTTRYNQKFKG

SEQ ID NO: 943 - protein, 7 aa, synthetic construct
GLNAWDY

SEQ ID NO: 944 - protein, 11 aa, synthetic construct
GASENIYGALN

SEQ ID NO: 945 - protein, 7 aa, synthetic construct
GATNLED

SEQ ID NO: 946 - protein, 9 aa, synthetic construct
QNVLTTPYT

SEQ ID NO: 947 - protein, 116 aa, synthetic construct; identical to SEQ ID NO: 532

SEQ ID NO: 948 - protein, 107 aa, synthetic construct; identical to SEQ ID NO: 533

SEQ ID NO: 949 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 950 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 951 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 952 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 953 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 954 - protein, 214 aa, synthetic construct; identical to SEQ ID NO: 951

SEQ ID NO: 955 - protein, 7 aa, synthetic construct
SGSYYWG

SEQ ID NO: 956 - protein, 16 aa, synthetic construct
NIYYSGSTYYNPSLRS

SEQ ID NO: 957 - protein, 10 aa, synthetic construct
EGSYPNQFDP

SEQ ID NO: 958 - protein, 11 aa, synthetic construct
RASQSVSSNLA

SEQ ID NO: 959 - protein, 7 aa, synthetic construct
GASTRAT

SEQ ID NO: 960 - protein, 9 aa, synthetic construct
QQYHSFPFT

SEQ ID NO: 961 - protein, 120 aa, synthetic construct; identical to SEQ ID NO: 382

SEQ ID NO: 962 - protein, 107 aa, synthetic construct; identical to SEQ ID NO: 383

SEQ ID NO: 963 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 964 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 965 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 966 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 967 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 968 - protein, 214 aa, synthetic construct; identical to SEQ ID NO: 965

SEQ ID NO: 969 - protein, 11 aa, synthetic construct
GSISSSSYYWG

SEQ ID NO: 970 - protein, 16 aa, synthetic construct
NIYYSGSTYYNPSLKS

SEQ ID NO: 971 - protein, 12 aa, synthetic construct
AREGSYPNWFDP

SEQ ID NO: 972 - protein, 11 aa, synthetic construct
RASQSVSSNLA

SEQ ID NO: 973 - protein, 7 aa, synthetic construct
GASTRAT

SEQ ID NO: 974 - protein, 9 aa, synthetic construct
QQYHSFPFT

SEQ ID NO: 975 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSS

SEQ ID NO: 976 - protein, 107 aa, synthetic construct; identical to SEQ ID NO: 383

SEQ ID NO: 977 - protein, 11 aa, synthetic construct
GSIKSGSHYWG

SEQ ID NO: 978 - protein, 16 aa, synthetic construct
NIYYSGSTYYNPSLRS

SEQ ID NO: 979 - protein, 12 aa, synthetic construct
AREGSYPNWFDP

SEQ ID NO: 980 - protein, 11 aa, synthetic construct
RASQSVSSNLA

SEQ ID NO: 981 - protein, 7 aa, synthetic construct
GASTRAT

SEQ ID NO: 982 - protein, 9 aa, synthetic construct
QQYHSFPFT

SEQ ID NO: 983 - protein, 120 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSS

SEQ ID NO: 984 - protein, 107 aa, synthetic construct; identical to SEQ ID NO: 383

SEQ ID NO: 985 - protein, 11 aa, synthetic construct
GSIKSGSHYWG

SEQ ID NO: 986 - protein, 16 aa, synthetic construct
NIYYSGSTYYNPSLKS

SEQ ID NO: 987 - protein, 12 aa, synthetic construct
AREGSYPNWLDP

SEQ ID NO: 988 - protein, 11 aa, synthetic construct
RASQSVSSNLA

SEQ ID NO: 989 - protein, 7 aa, synthetic construct
GASTRAT

SEQ ID NO: 990 - protein, 9 aa, synthetic construct
QQYHSFPFT

SEQ ID NO: 991 - protein, 120 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSS

SEQ ID NO: 992 - protein, 107 aa, synthetic construct; identical to SEQ ID NO: 383

SEQ ID NO: 993 - protein, 11 aa, synthetic construct
GSIKSGSYYWG

SEQ ID NO: 994 - protein, 16 aa, synthetic construct
NIYYSGSTYYNPSLKS

SEQ ID NO: 995 - protein, 12 aa, synthetic construct
AREGSYPNQFDP

SEQ ID NO: 996 - protein, 11 aa, synthetic construct
RASQSVSSNLA

SEQ ID NO: 997 - protein, 7 aa, synthetic construct
GASTRAT

SEQ ID NO: 998 - protein, 9 aa, synthetic construct
QQYHSFPFT

SEQ ID NO: 999 - protein, 120 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSS

SEQ ID NO: 1000 - protein, 107 aa, synthetic construct; identical to SEQ ID NO: 383

SEQ ID NO: 1001 - protein, 11 aa, synthetic construct
GSIKSGSHYWG

SEQ ID NO: 1002 - protein, 16 aa, synthetic construct
NIYYSGSTYYNPSLKS

SEQ ID NO: 1003 - protein, 12 aa, synthetic construct
AREGSYPNWFDP

SEQ ID NO: 1004 - protein, 11 aa, synthetic construct
RASQSVSTNLA

SEQ ID NO: 1005 - protein, 7 aa, synthetic construct
DASARVT

SEQ ID NO: 1006 - protein, 9 aa, synthetic construct
QQYHSFPFT

SEQ ID NO: 1007 - protein, 120 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSS

SEQ ID NO: 1008 - protein, 107 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIK

SEQ ID NO: 1009 - protein, 11 aa, synthetic construct
GSISSSSYYWG

SEQ ID NO: 1010 - protein, 16 aa, synthetic construct
NIYYSGSTYYNPSLKS

SEQ ID NO: 1011 - protein, 12 aa, synthetic construct
AREGSYTTVLNV

SEQ ID NO: 1012 - protein, 12 aa, synthetic construct
RASQSVSSSYLA

SEQ ID NO: 1013 - protein, 7 aa, synthetic construct
GASSRAT

SEQ ID NO: 1014 - protein, 9 aa, synthetic construct
QQAASYPLT

SEQ ID NO: 1015 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 1016 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIK

SEQ ID NO: 1017 - protein, 11 aa, synthetic construct
GSIGRGSYYWG

SEQ ID NO: 1018 - protein, 16 aa, synthetic construct
NIYYSGSTYYNPSLKS

SEQ ID NO: 1019 - protein, 12 aa, synthetic construct
AREGSYTTVLNV

SEQ ID NO: 1020 - protein, 12 aa, synthetic construct
RASQSVASSHLA

SEQ ID NO: 1021 - protein, 7 aa, synthetic construct
DAVSRAT

SEQ ID NO: 1022 - protein, 9 aa, synthetic construct
QQAASYPLT

SEQ ID NO: 1023 - protein, 120 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 1024 - protein, 108 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIK

SEQ ID NO: 1025 - protein, 11 aa, synthetic construct
GSISSGGYYWS

SEQ ID NO: 1026 - protein, 16 aa, synthetic construct
NIYYSGSTYYNPSLKS

SEQ ID NO: 1027 - protein, 13 aa, synthetic construct
ARESSTISADFDL

SEQ ID NO: 1028 - protein, 11 aa, synthetic construct
RASQGISRWLA

SEQ ID NO: 1029 - protein, 7 aa, synthetic construct
AASSLQS

SEQ ID NO: 1030 - protein, 9 aa, synthetic construct
QQAHTFPYT

SEQ ID NO: 1031 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSS

SEQ ID NO: 1032 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIK

SEQ ID NO: 1033 - protein, 11 aa, synthetic construct
GSISHGGYYWS

SEQ ID NO: 1034 - protein, 16 aa, synthetic construct
NIYYSGSTYYNPSLKS

SEQ ID NO: 1035 - protein, 13 aa, synthetic construct
ARESSTISADFDL

SEQ ID NO: 1036 - protein, 11 aa, synthetic construct
RASQGISRWLA

SEQ ID NO: 1037 - protein, 7 aa, synthetic construct
AASSLQS

SEQ ID NO: 1038 - protein, 9 aa, synthetic construct
QQAHTFPYT

SEQ ID NO: 1039 - protein, 121 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSS

SEQ ID NO: 1040 - protein, 107 aa, synthetic construct; identical to SEQ ID NO: 1032

SEQ ID NO: 1041 - protein, 11 aa, synthetic construct
GSISSGGYYWS

SEQ ID NO: 1042 - protein, 16 aa, synthetic construct
NIYYSGSTYYNPSLKS

SEQ ID NO: 1043 - protein, 13 aa, synthetic construct
ARGLSTIDEAFDP

SEQ ID NO: 1044 - protein, 11 aa, synthetic construct
RASQSISSWLA

SEQ ID NO: 1045 - protein, 7 aa, synthetic construct
KASSLES

SEQ ID NO: 1046 - protein, 9 aa, synthetic construct
QQDNSYPYT

SEQ ID NO: 1047 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSS

SEQ ID NO: 1048 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIK

SEQ ID NO: 1049 - protein, 11 aa, synthetic construct
GSISDGSYYWS

SEQ ID NO: 1050 - protein, 16 aa, synthetic construct
NIYYSGSTYYNPSLRS

SEQ ID NO: 1051 - protein, 13 aa, synthetic construct
ARGLSTIDEAFDP

SEQ ID NO: 1052 - protein, 11 aa, synthetic construct
RASQSISSWLA

SEQ ID NO: 1053 - protein, 7 aa, synthetic construct
KASSLES

SEQ ID NO: 1054 - protein, 9 aa, synthetic construct
QQDNSYPYT

SEQ ID NO: 1055 - protein, 121 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 1056 - protein, 107 aa, synthetic construct; identical to SEQ ID NO: 1048

SEQ ID NO: 1057 - protein, 11 aa, synthetic construct
GSISDGSYYWS

SEQ ID NO: 1058 - protein, 16 aa, synthetic construct
NIYYSGSTYYNPSLRS

SEQ ID NO: 1059 - protein, 13 aa, synthetic construct
ARGLSTIDEAFDP

SEQ ID NO: 1060 - protein, 11 aa, synthetic construct
RASKSISSWLA

SEQ ID NO: 1061 - protein, 7 aa, synthetic construct
EASSLHS

SEQ ID NO: 1062 - protein, 9 aa, synthetic construct
QQDNSYPYT

SEQ ID NO: 1063 - protein, 121 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 1064 - protein, 107 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIK

SEQ ID NO: 1065 - protein, 9 aa, synthetic construct
GSISSYYWS

SEQ ID NO: 1066 - protein, 16 aa, synthetic construct
YIYSSGSTNYNPSLKS

SEQ ID NO: 1067 - protein, 15 aa, synthetic construct
ARGSGQYAAPDYGMD

SEQ ID NO: 1068 - protein, 11 aa, synthetic construct
RASQSISSWLA

SEQ ID NO: 1069 - protein, 7 aa, synthetic construct
KASSLES

SEQ ID NO: 1070 - protein, 9 aa, synthetic construct
QQDNSFPFT

SEQ ID NO: 1071 - protein, 122 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSS

SEQ ID NO: 1072 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIK

SEQ ID NO: 1073 - protein, 9 aa, synthetic construct
GSIISYYWG

SEQ ID NO: 1074 - protein, 16 aa, synthetic construct
YIYSSGSTSYNPSLKS

SEQ ID NO: 1075 - protein, 16 aa, synthetic construct
ARGSGLYAAPDYGLDV

SEQ ID NO: 1076 - protein, 11 aa, synthetic construct
RASQSISSWLA

SEQ ID NO: 1077 - protein, 7 aa, synthetic construct
KASSLES

SEQ ID NO: 1078 - protein, 9 aa, synthetic construct
QQDNSFPFT

SEQ ID NO: 1079 - protein, 122 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSS

SEQ ID NO: 1080 - protein, 107 aa, synthetic construct; identical to SEQ ID NO: 1072

SEQ ID NO: 1081 - protein, 9 aa, synthetic construct
FTFSSYAMS

SEQ ID NO: 1082 - protein, 17 aa, synthetic construct
TISGSGGSTYYADSVKG

SEQ ID NO: 1083 - protein, 13 aa, synthetic construct
ARGAGHYDLVGRY

SEQ ID NO: 1084 - protein, 11 aa, synthetic construct
RASQSISSYLN

SEQ ID NO: 1085 - protein, 7 aa, synthetic construct
AASSLQS

SEQ ID NO: 1086 - protein, 9 aa, synthetic construct
QQLYSLPPT

SEQ ID NO: 1087 - protein, 120 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSS

SEQ ID NO: 1088 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIK

SEQ ID NO: 1089 - protein, 9 aa, synthetic construct
FTFSSYAMS

SEQ ID NO: 1090 - protein, 17 aa, synthetic construct
AISGSGGSTYYADSVKG

SEQ ID NO: 1091 - protein, 10 aa, synthetic construct
ARVGFRALNY

SEQ ID NO: 1092 - protein, 11 aa, synthetic construct
RASQDISSWLA

SEQ ID NO: 1093 - protein, 7 aa, synthetic construct
AASSLQS

SEQ ID NO: 1094 - protein, 10 aa, synthetic construct
QQATSYPPWT

SEQ ID NO: 1095 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSS

SEQ ID NO: 1096 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIK

SEQ ID NO: 1097 - protein, 9 aa, synthetic construct
GTFSSYAIS

SEQ ID NO: 1098 - protein, 17 aa, synthetic construct
GIIPIFGTASYAQKFQG

SEQ ID NO: 1099 - protein, 13 aa, synthetic construct
ARQQYDGRRYFGL

SEQ ID NO: 1100 - protein, 11 aa, synthetic construct
RASQSVSSNLA

SEQ ID NO: 1101 - protein, 7 aa, synthetic construct
SASTRAT

SEQ ID NO: 1102 - protein, 9 aa, synthetic construct
QQVNVWPPT

SEQ ID NO: 1103 - protein, 120 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSS

SEQ ID NO: 1104 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIK

SEQ ID NO: 1105 - protein, 9 aa, synthetic construct
GTFSSYAIS

SEQ ID NO: 1106 - protein, 17 aa, synthetic construct
GIIPIFGTANYAQKFQG

SEQ ID NO: 1107 - protein, 9 aa, synthetic construct
ARGGPWFDP

SEQ ID NO: 1108 - protein, 11 aa, synthetic construct
RASQSISSWLA

SEQ ID NO: 1109 - protein, 7 aa, synthetic construct
KASSLES

SEQ ID NO: 1110 - protein, 10 aa, synthetic construct
QQYNSYPPFT

SEQ ID NO: 1111 - protein, 116 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSS

SEQ ID NO: 1112 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIK

SEQ ID NO: 1113 - protein, 9 aa, synthetic construct
FTFSSYAMS

SEQ ID NO: 1114 - protein, 17 aa, synthetic construct
AISGSGGSTSYADSVKG

SEQ ID NO: 1115 - protein, 13 aa, synthetic construct
AKPSLATMLAFDI

SEQ ID NO: 1116 - protein, 11 aa, synthetic construct
RASQSISSWLA

SEQ ID NO: 1117 - protein, 7 aa, synthetic construct
DASSLES

SEQ ID NO: 1118 - protein, 9 aa, synthetic construct
QQSKSYPRT

SEQ ID NO: 1119 - protein, 120 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSS

SEQ ID NO: 1120 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIK

SEQ ID NO: 1121 - protein, 11 aa, synthetic construct
GSISSSVYYWS

SEQ ID NO: 1122 - protein, 16 aa, synthetic construct
SILVSGSTYYNPSLKS

SEQ ID NO: 1123 - protein, 9 aa, synthetic construct
ARAVSFLDV

SEQ ID NO: 1124 - protein, 11 aa, synthetic construct
RASQSISSYLN

SEQ ID NO: 1125 - protein, 7 aa, synthetic construct
GASSLQS

SEQ ID NO: 1126 - protein, 9 aa, synthetic construct
QQSYDPPWT

SEQ ID NO: 1127 - protein, 117 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSS

SEQ ID NO: 1128 - protein, 107 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIK

SEQ ID NO: 1129 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1130 - protein, 214 aa, synthetic construct; identical to SEQ ID NO: 965

SEQ ID NO: 1131 - protein, 450 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1132 - protein, 214 aa, synthetic construct; identical to SEQ ID NO: 965

SEQ ID NO: 1133 - protein, 450 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1134 - protein, 214 aa, synthetic construct; identical to SEQ ID NO: 965

SEQ ID NO: 1135 - protein, 450 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1136 - protein, 214 aa, synthetic construct; identical to SEQ ID NO: 965

SEQ ID NO: 1137 - protein, 450 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1138 - protein, 214 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1139 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1140 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1141 - protein, 450 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1142 - protein, 215 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1143 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1144 - protein, 214 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1145 - protein, 451 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1146 - protein, 214 aa, synthetic construct; identical to SEQ ID NO: 1144

SEQ ID NO: 1147 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1148 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1149 - protein, 451 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1150 - protein, 214 aa, synthetic construct; identical to SEQ ID NO: 1148

SEQ ID NO: 1151 - protein, 451 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1152 - protein, 214 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1153 - protein, 452 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1154 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1155 - protein, 452 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1156 - protein, 214 aa, synthetic construct; identical to SEQ ID NO: 1154

SEQ ID NO: 1157 - protein, 450 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1158 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1159 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1160 - protein, 215 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1161 - protein, 450 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1162 - protein, 214 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1163 - protein, 446 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1164 - protein, 215 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1165 - protein, 450 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1166 - protein, 214 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1167 - protein, 447 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1168 - protein, 214 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1169 - protein, 5 aa, synthetic construct
NYGMN

SEQ ID NO: 1170 - protein, 17 aa, synthetic construct
WINTYTGEPTYADAFKG

SEQ ID NO: 1171 - protein, 9 aa, synthetic construct
DYGDYGMDY

SEQ ID NO: 1172 - protein, 15 aa, synthetic construct
RASKSVSTSGYSFMH

SEQ ID NO: 1173 - protein, 7 aa, synthetic construct
LASNLES

SEQ ID NO: 1174 - protein, 9 aa, synthetic construct
QHSREVPWT

SEQ ID NO: 1175 - protein, 450 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1176 - protein, 213 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1177 - protein, 449 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1178 - protein, 219 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1179 - protein, 449 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1180 - protein, 215 aa, synthetic construct
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1181 - protein, 18 aa, synthetic construct
DMGWGSGWRPYYYYGMDV

SEQ ID NO: 1182 - protein, 111 aa, synthetic construct
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVL

SEQ ID NO: 1183 - protein, 5 aa, synthetic construct
SYWMH

SEQ ID NO: 1184 - protein, 5 aa, synthetic construct
SYWMH

SEQ ID NO: 1185 - protein, 15 aa, synthetic construct
KASQSVDYSGDSYMN

SEQ ID NO: 1186 - protein, 5 aa, synthetic construct
SYGMN

SEQ ID NO: 1187 - protein, 17 aa, synthetic construct
YISSSSSTIYYADSVKG

SEQ ID NO: 1188 - protein, 114 aa, synthetic construct
QVQLQQSGGGVVQPGRSLGLSCAASGFTFSSYGMNWVRQAPGKGLEWVSYISSSSSTIYYADSVKGRFTISRDNSKNTLYLQMNSLRAEDTAVYYCARGPGMDVWGQGTTVTVS

SEQ ID NO: 1189 - protein, 17 aa, synthetic construct
YISSDSSAIYYADTVKG

SEQ ID NO: 1190 - protein, 13 aa, synthetic construct
GRENIYYGSRLDY

SEQ ID NO: 1191 - protein, 11 aa, synthetic construct
KASQNVDTNVA

SEQ ID NO: 1192 - protein, 7 aa, synthetic construct
SASYRYS

SEQ ID NO: 1193 - protein, 9 aa, synthetic construct
QQYNNYPFT

SEQ ID NO: 1194 - protein, 5 aa, synthetic construct
SYGMS

SEQ ID NO: 1195 - protein, 17 aa, synthetic construct
TINSGGSNTYYPDSLKG

SEQ ID NO: 1196 - protein, 8 aa, synthetic construct
HDGGAMDY

SEQ ID NO: 1197 - protein, 11 aa, synthetic construct
RASESIYSYLA

SEQ ID NO: 1198 - protein, 10 aa, synthetic construct
QHHYGTPPWT

SEQ ID NO: 1199 - protein, 5 aa, synthetic construct
SFGMH

SEQ ID NO: 1200 - protein, 17 aa, synthetic construct
YISSGSGTIYYADTVKG

SEQ ID NO: 1201 - protein, 10 aa, synthetic construct
HGYRYEGFDY

SEQ ID NO: 1202 - protein, 11 aa, synthetic construct
KASQNVDTNVA

SEQ ID NO: 1203 - protein, 7 aa, synthetic construct
SASYRYS

SEQ ID NO: 1204 - protein, 9 aa, synthetic construct
QQYNNYPFT

SEQ ID NO: 1205 - protein, 5 aa, synthetic construct
SYTIH

SEQ ID NO: 1206 - protein, 106 aa, synthetic construct
DIQLTQSPSFLSASVGDRVTITCRASSSVSYMNWYQQKPGKSPKPWIYATSNLASGVPSRFSVSVSGTEHTLTISSLQPEDFATYYCQQWSSNPLTFGQGTKLEIK

SEQ ID NO: 1207 - protein, 5 aa, synthetic construct
SYWMH

SEQ ID NO: 1208 - protein, 8 aa, synthetic construct
GGRLYFDY

SEQ ID NO: 1209 - protein, 5 aa, synthetic construct
SYWMH

SEQ ID NO: 1210 - protein, 112 aa, synthetic construct
DIVMTQSPLSLPVTPGEPASISCRSSQSLVHSNQDTYLRWYLQKPGQSPQLLIYKVSNRFSGVPDRFSGSGSGTDFTLKISRVEAEDVGVYYCSQSTHVPYTFGGGTKVEIK

SEQ ID NO: 1211 - protein, 6 aa, synthetic construct
SGYSWH

SEQ ID NO: 1212 - protein, 107 aa, synthetic construct; identical to SEQ ID NO: 231

SEQ ID NO: 1213 - protein, 17 aa, synthetic construct
WIFPGDDSTQYNEKFKG

SEQ ID NO: 1214 - protein, 9 aa, synthetic construct
QNGHSFPLT

SEQ ID NO: 1215 - protein, 106 aa, synthetic construct
DIQMTQSPSSLSASVGDRVTITCSVSSSVFYVHWYQQKPGKAPKLLIYDTSKLASGVPSRFSGSGSGTDFTFTISSLQPEDIATYYCQQWNSNPPTFGGGTKVEIK

SEQ ID NO: 1216 - protein, 106 aa, synthetic construct
QIVLTQSPAIMSASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 1217 - protein, 19 aa, synthetic construct
DGVLRYFDWLLDYYYYMDV

SEQ ID NO: 1218 - protein, 17 aa, synthetic construct
GIDPETGGTAYNQKFKG

SEQ ID NO: 1219 - protein, 16 aa, synthetic construct
RSSQSIVHSNANTYLQ

SEQ ID NO: 1220 - protein, 16 aa, synthetic construct
GPSEVGAILGYVWFDP

SEQ ID NO: 1221 - protein, 106 aa, synthetic construct
DIQMTQSPSSLSASVGDRVTINCKASQDINSYLSWFQQKPGKAPKTLIYRANRVDGVPSRFSGSGSGQDYTLTISSLQPEDFATYYCLQYDEFPLTFGGGTKVEIK

SEQ ID NO: 1222 - protein, 4 aa, synthetic construct
GGGG

SEQ ID NO: 1223 - protein, 4 aa, synthetic construct
GVKG

SEQ ID NO: 1224 - protein, 4 aa, synthetic construct
VKGG

SEQ ID NO: 1225 - protein, 4 aa, synthetic construct
GGFG

SEQ ID NO: 1226 - protein, 5 aa, synthetic construct
GGFGG